(19) United States
(12) Patent Application Publication
(10) Pub. No.: US 2016/0272707 A1
(43) Pub. Date: Sep. 22, 2016

(54) VSTM5 ANTIBODIES, AND USES THEREOF FOR TREATMENT OF CANCER, INFECTIOUS DISEASES AND IMMUNE RELATED DISEASES

(71) Applicant: COMPUGEN LTD., Holon (IL)

(72) Inventors: Zurit LEVINE, Herzliyya (IL); Galit ROTMAN, Herzliyya (IL); Liat DASSA, Yehud (IL); Ofer LEVY, Moshav Mesisraelat Zion (IL); Gad S. COJOCARU, Ramat HaSharon (IL); Amir TOPORIK, Pardes Hannah Karkur (IL); Yossef KLIGER, Rishon LeZiyyon (IL); Andrew POW, San Francisco, CA (US); Spencer LIANG, San Mateo, CA (US)

(21) Appl. No.: 15/021,239

(22) PCT Filed: Sep. 11, 2014
(86) PCT No.: PCT/IL2014/050814

§ 371 Date: Mar. 10, 2016

Related U.S. Application Data
(60) Provisional application No. 61/876,324, filed on Sep. 11, 2013.

(51) Int. Cl. C07K 16/28; A61K 31/664; C07K 16/30; A61K 39/00; A61K 39/395; A61K 45/06

(52) U.S. Cl. CPC C07K 16/28; A61K 39/39558; A61K 45/06; C07K 16/30; A61K 39/0011; C07K 16/2818; A61K 31/664; A61K 39/3955; C07K 2317/76; C07K 2317/33; C07K 2317/94; C07K 2317/92; C07K 2317/55; C07K 2317/565; C07K 2317/51; C07K 2317/515; C07K 2317/21; C07K 2317/73; A61K 2039/505; A61K 2039/507; A61K 2039/5154

(57) ABSTRACT

The present invention relates to VSTM5-specific antibodies, antibody fragments, and VSTM5 polypeptides, conjugates and compositions comprising same, for modulating (antagonizing or agonizing) one or more of the effects of VSTM5 expression on immunity. More specifically, the present invention relates to VSTM5-specific antibodies, antibody fragments, and VSTM5 polypeptides, conjugates and compositions comprising same for treating and aiding in the diagnosis of cancer, infectious diseases and immune related diseases, e.g., those associated with aberrant (higher or lower than normal) VSTM5 expression by diseased and/or immune cells and/or aberrant (increased or reduced) VSTM5-mediated effects on immunity.

### FIELD OF THE INVENTION

The present invention, in at least some aspects, relates to anti-VSTM5 antibodies, antigen-binding fragments, conjugates thereof, and compositions containing such which modulate (agonize or antagonize) the effects of VSTM5 on immunity, as well as methods of production and therapeutic use thereof.

### BACKGROUND OF THE INVENTION

Naïve T cells must receive two independent signals from antigen-presenting cells (APC) in order to become productively activated. The first, Signal 1, is antigen-specific and occurs when T cell antigen receptors encounter the appropriate antigen-MHC complex on the APC. The fate of the immune response is determined by a second, antigen-independent signal (Signal 2) which is delivered through a T cell costimulatory molecule that engages its APC-expressed ligand. This second signal could be either stimulatory (positive costimulation) or inhibitory (negative costimulation or coinhibition). In the absence of a costimulatory signal, or in the presence of a coinhibitory signal, T-cell activation is impaired or aborted, which may lead to a state of antigen-specific unresponsiveness (known as T-cell anergy), or may result in T-cell apoptotic death.

Costimulatory molecule pairs usually consist of ligands expressed on APCs and their cognate receptors expressed on T cells. The prototype ligand/receptor pairs of costimulatory molecules are B7/CD28 and CD40/CD40L. The B7 family consists of structurally related, cell-surface protein ligands, which may provide stimulatory or inhibitory input to an immune response. Members of the B7 family are structurally related, with the extracellular domain containing at least one variable or constant immunoglobulin domain.

Both positive and negative costimulatory signals play critical roles in the regulation of cell-mediated immune responses, and molecules that mediate these signals have proven to be effective targets for immunomodulation. Based on this knowledge, several therapeutic approaches that involve targeting of costimulatory molecules have been developed, and were shown to be useful for prevention and treatment of cancer by turning on, or preventing the turning off, of immune responses in cancer patients and for prevention and treatment of autoimmune diseases and inflammatory diseases, as well as rejection of allogenic transplantation, each by turning off uncontrolled immune responses, or by induction of “off signal” by negative costimulation (or coinhibition) in subjects with these pathological conditions.

Manipulation of the signals delivered by B7 ligands has shown potential in the treatment of autoimmunity, inflammatory diseases, and transplant rejection. Therapeutic strategies include blocking of costimulation using monoclonal antibodies to the ligand or to the receptor of a costimulatory pair, or using soluble fusion proteins composed of the costimulatory receptor that may bind and block its appropriate ligand. Another approach is induction of co-inhibition using soluble fusion protein of an inhibitory ligand. These approaches rely, at least partially, on the eventual deletion of auto- or allo-reactive T cells (which are responsible for the pathogenic processes in autoimmune diseases or transplantation, respectively), presumably because in the absence of costimulation (which induces cell survival genes) T cells become highly susceptible to induction of apoptosis. Thus, novel agents that are capable of modulating costimulatory signals, without compromising the immune system's ability to defend against pathogens, are highly advantageous for treatment and prevention of such pathological conditions.

Costimulatory pathways play an important role in tumor development. Interestingly, tumors have been shown to evade immune destruction by impeding T cell activation through inhibition of co-stimulatory factors in the B7-CD28 and TNF families, as well as by attracting regulatory T cells, which inhibit anti-tumor T cell responses (see Wang (2006), “Immune Suppression by Tumor Specific CD4⁺ Regulatory T cells in Cancer”, Semin. Cancer. Biol. 16:73-79; Greenwald, et al. (2005), “The B7 Family Revisited”, Ann. Rev. Immunol. 23:515-48; Watts (2005), “TNF/TNFR Family Members in Co-stimulation of T Cell Responses”, Ann. Rev. Immunol. 23:23-68; Sadum, et al., (2007) “Immune Signatures of Murine and Human Cancers Reveal Unique Mechanisms of Tumor Escape and New Targets for Cancer Immunotherapy”, Clin. Canc. Res. 13(13): 4016-4025). Such tumor expressed co-stimulatory molecules have become attractive cancer biomarkers and may serve as tumor-associated antigens (TAAs). Furthermore, costimulatory pathways have been identified as immunologic checkpoints that attenuate T cell dependent immune responses, both at the level of initiation and effector function within tumor metastases. As engineered cancer vaccines continue to improve, it is becoming clear that such immunologic checkpoints are a major barrier to the vaccines' ability to induce therapeutic anti-tumor responses. In that regard, costimulatory molecules can serve as adjuvants for active (vaccination) and passive (antibody-mediated) cancer immunotherapy, providing strategies to thwart immune tolerance and stimulate the immune system.

Over the past decade, agonists and/or antagonists to various costimulatory proteins have been developed for treating autoimmune diseases, graft rejection, allergy and cancer. For example, CTLA4-Ig (Abatacept, Orencia®) is approved for treatment of RA, mutated CTLA4-Ig (Belatacept, Nulojix®) for prevention of acute kidney transplant rejection and by the anti-CTLA4 antibody (Ipilimumab, Yervoy®), recently approved for the treatment of melanoma. Other costimulation regulators are currently in advanced stages of clinical development including anti-PD-1 antibody (BMS-936558) which is in development for treatment of Non-Small Cell Lung cancer and other cancers. Furthermore, such agents are also in clinical development for viral infections, for example the anti PD-1 Ab, MDX-1106, which is being tested for treatment of hepatitis C, and the anti-CTLA-4 Ab CP-675,206 (tremelimumab) for use in hepatitis C virus-infected patients with hepatocellular carcinoma.

### BRIEF SUMMARY OF THE INVENTION

The present invention in some embodiments relates to the demonstration that VSTM5 elicits specific effects on immunity, in particular that this polypeptide has an effect on specific types of immune cells and the production of cytokines which are involved in adaptive immunity, especially antitumor immunity and immune reactions to infectious agents as well as immune related diseases. Specifically, it is shown herein that VSTM5 elicits an inhibitory effect on T cell activation and proliferation, an inhibitory effect on cytotoxic T lymphocyte (CTL) immunity and CTL-directed killing of target cells, e.g., cancer cells, an inhibitory effect on CD4⁺ T cell immunity and on antigen-specific CD4⁺ T cell immunity, an inhibitory effect on natural killer (NK) cell mediated killing of target cells, an inhibitory effect on the secretion of certain cytokines such as IL-2, INFN-γ and TNF-α by T cells, and a potentiating effect on the induction or differentiation and proliferation of inducible T regulatory or suppressor cells (iTregs) (which cells are known to be involved in eliciting tolerance to self-antigens and to suppress anti-tumor immunity). Also, the present invention, in at least some embodiments, relates to the discovery that antibodies and antigen-binding fragments may be obtained which modulate (agonize or antagonize) one or more of the effects of VSTM5 on immunity, and that such antibodies and antigen-binding fragments may be used to upregulate or down-regulate immunity and be used in treating diseases such as cancer, infection, sepsis, autoimmunity, inflammation, allergic and other immune conditions.

The present invention, in at least some embodiments, relates to anti-VSTM5 antibodies, antigen-binding fragments, conjugates thereof, and compositions containing which modulate (agonize or antagonize) the effects of VSTM5 on immunity. Also, the invention relates to screening methods for identifying anti-VSTM5 antibodies that modulate the effects of VSTM5 on immunity and antibodies obtained by such screening methods. Further, the present invention in at least some embodiments relates to diagnostic and therapeutic compositions comprising same, and the use thereof for modulating (antagonizing or agonizing) one or more of the effects of VSTM5 on immunity and/or for detecting disease conditions wherein VSTM5 expression correlates to the disease, or risk of the disease, and/or may elicit an effect on immunity.

According to at least some embodiments, the present invention relates to anti-VSTM5 antibodies, antigen-binding fragments, conjugates and compositions comprising same for treating and aiding in the diagnosis of cancer, infectious diseases, sepsis and immune related diseases such as autoimmune, allergic and inflammatory conditions, e.g., conditions associated with VSTM5 expression by diseased, stromal or antigen-presenting cells, optionally wherein the endogenous disease pathology is enhanced or inhibited by VSTM5-mediated effects on immunity.

Related thereto, the present invention according to at least some embodiments provides immunomodulatory (immunostimulatory or immunoinhibitory) VSTM5-specific antibodies, antigen-binding fragments, conjugates and compositions comprising same, for modulating (antagonizing or agonizing) one or more of the effects of VSTM5 on immunity. Preferably, these antibodies and polypeptides will be suitable for use in human therapy, e.g., for treating and aiding in the diagnosis of cancer, infectious disease, sepsis, and immune diseases such as autoimmune, allergic and inflammatory conditions, including conditions associated with aberrant VSTM5 expression and VSTM5-mediated effects on immunity.

As VSTM5 has a suppressive effect on immune cells such as CD4⁺ T cells, CD8⁺ or CTLs and NK cells, which cells are known to be involved in killing of pathological or diseased cells such as cancer and infected cells and pathogens, but without wishing to be limited by a single hypothesis, antibodies, and antigen-binding fragments and conjugates thereof which antagonize the inhibitory effects of VSTM5 on T cell or NK cell-mediated immunity are expected to be well suited for the treatment of cancer, infectious diseases and sepsis and other indications wherein enhanced immune responses and/or the depletion of target cells is therapeutically desired. Also, these immunomodulatory VSTM5 specific antibodies and antibody fragments and polypeptides which antagonize VSTM5, again pathological or diseased cells such as cancer and infected cells and pathogens, but without wishing to be limited by a single hypothesis, are expected to be useful as immune adjuvants in therapeutic vaccine formulations, e.g., anticancer vaccines, antivirus vaccines and other therapeutic vaccine formulations which contain an antigen specific to a target cell such as a cancerous cell or infectious agent.

Moreover, as VSTM5 has an inhibitory effect on specific immune cells such as CD4⁺ T cells, CD8⁺ T cells or CTLs, and NK cells, which cells are known to be involved in the pathology of certain immune conditions such as autoimmune and inflammatory disorders, as well as eliciting a potentiating effect on iTregs, antibodies, antigen-binding fragments and conjugates thereof which potentiate or agonize the effects of VSTM5 on immunity, again pathological or diseased cells such as cancer and infected cells and pathogens, but without wishing to be limited by a single hypothesis, are expected to be well suited for treating conditions wherein the suppression of T cell or NK mediated immunity and/or the induction of immune tolerance or prolonged suppression of antigen-specific immunity is therapeutically desirable, e.g., the treatment of autoimmune, inflammatory or allergic conditions, and the suppression of undesired immune responses such as to cell or gene therapy or organ and tissue transplantation and graft versus host disease (GVHD).

Based thereon, in some embodiments the present invention provides VSTM5-specific antibodies, antigen-binding fragments, conjugates and compositions comprising same, and methods of use thereof for drug development, for treatment of cancer, infectious diseases, sepsis, as well as immune related diseases such as autoimmune, allergic and inflammatory conditions and/or for reducing the undesirable immune activation that may be associated with cell or gene therapy, and tissue or organ transplantation associated conditions.

Particularly, according to at least some embodiments the present invention provides novel antibodies, antigen-binding fragments, conjugates thereof, and compositions containing that upregulate or downregulate immunity and the use thereof in treating conditions wherein upregulation or downregulation of immunity is therapeutically desired, especially chronic conditions such as cancer wherein antibodies, because of their long in vivo half-life, may elicit a prolonged effect on immunity. The subject immunostimulatory antibodies, based on their stimulatory effect on T cell and NK cell immunity and suppressive effect on T_{Regs} may be used to treat different cancers, including those where a suitable therapies are presently unavailable or not very effective, i.e., by stimulating the host's innate immune system against tumors. Also, there is a need for new cancer therapies that do not include or require the use of chemotherapeutics or radiation, or other current cancer treatments, which while killing cancer cells, may elicit undesired effects such as killing of non-target cells or even causing cancer reoccurrence. However it should be noted that such embodiments are optional and that optionally, an antibody, fragment, conjugate and so forth as described herein may optionally be used in combination with a known, different anti-cancer therapy.

Moreover, according to at least some embodiments the subject immunopotentiating anti-VSTM5 antibodies (i.e., antibodies that antagonize the inhibitory effects of VSTM5 on T cell or NK cell-mediated immunity and thereby potentiate immune responses) and antigen-binding fragments thereof, based on their immunopotentiating effects, but without wishing to be limited by a single hypothesis, may optionally be used to treat different cancer conditions alone or in combination with other conventional therapies and active agents such as other immunomodulatory compounds, chemotherapy, radiation and the like as the subject immunostimulatory antibodies may potentiate the therapeutic effects of such actives by inhibiting VSTM5-mediated immunosuppression of the treated subject's innate (e.g., anti-tumor) immunity.

Further, given the recent increase in infectious disease and the risk of the global spread of virulent infectious diseases, in particular viral diseases, antibiotic resistant bacterial strains, and sepsis, there is an urgent need for improved methods and compositions for treating infectious disease and sepsis. It is anticipated, without wishing to be limited by a single hypothesis, that anti-VSTM5 antibodies and antigen-binding fragments which antagonize the effects of VSTM5 on immunity may be used to effectively treat different infectious conditions including bacterial, parasite, yeast or fungal, myoplasm and viral infection, and treat or prevent sepsis, alone or in combination with other actives such as other immunomodulatory compounds.

Also, there has been an increase in the number of persons suffering from autoimmune, allergic and inflammatory conditions. Many of these conditions are not effectively treated and the disease symptoms are at best maintained by existing therapeutic regimens such as immunosuppressant drugs and biologics. Also, some drugs and biologics used to treat such conditions may themselves elicit undesired effects e.g., infectious conditions, sepsis or cancer because of prolonged immunosuppression. Therefore, there is a need for novel and improved drugs that effectively treat autoimmune, allergic and inflammatory conditions, or which may be used to inhibit or prevent undesired host immune responses during gene or cell therapy or prevent or ameliorate immune responses against transplanted tissues and organs and/or GVHD. The subject immunoinhibitory anti-VSTM5 antibodies and antigen-binding fragments, based on their immunosuppressive effects, may be used to effectively treat different immune conditions alone or in combination with other actives such as other immunosuppressive compounds and biologics.

Accordingly, the present invention in some embodiments is broadly directed to “immunomodulatory” anti-VSTM5 antibodies, antigen-binding fragments, conjugates and compositions containing same, preferably “immunomodulatory” anti-VSTM5 antibodies, antigen-binding fragments, conjugates and compositions containing same, and the use thereof in disease therapy and diagnosis. An “immunomodulatory” anti-VSTM5 antibody or antigen-binding fragment according to the invention encompasses any antibody or antigen-binding fragment that specifically binds VSTM5 that upregulates or downregulates at least one of the effects of VSTM5 on immunity, e.g., the inhibitory effects of VSTM5 on T or NK-cell mediated immunity.

Therefore, an “immunomodulatory” antibody or antigen-binding fragment according to the invention includes an “immunostimulatory antibody” or “immunostimulatory VSTM5 targeting antibody” or “immunostimulatory VSTM5 specific antibody”, used herein interchangeably, which inhibits one or more of the effects of VSTM5 on immune cells and hereby stimulates an immune response upon administration to a subject, in order to enhance immunity against cancer cells, infectious diseases, particularly chronic infections or sepsis Immunostimulatory antibodies comprise an expanding class of agents, which are either antagonists of immune-repressor molecules or agonists of immune-activating receptors. This new class of therapeutic agents has the ability to enhance anti-tumour immunity, comprising a new and promising strategy in cancer therapy.

Reduction of the immunoinhibitory activity of VSTM5 is especially desirable in situations in which VSTM5 itself (or biological systems into which it feeds or in which it participates) is abnormally upregulated, and/or situations in which decreased activity of VSTM5 leading to stimulation of immune responses is likely to have a beneficial effect, such as for example, immunotherapy and the treatment of cancer, infectious disorders and/or sepsis. Thus, as used herein, an “immunostimulatory VSTM5 targeting antibody” according to at least some embodiments of the present invention, is a therapeutic agent which reduces at least one VSTM5-mediated inhibitory activity on immune responses, leading to stimulation of immune responses. These immunopotentiating effects may be obtained by in vivo administration of such antibodies and antigen-binding fragments or may be obtained ex vivo, e.g., by contacting a patient cell sample or tissue or organ transplant with an immunostimulatory antibody or antigen-binding fragment according to the invention, which is then infused, re-infused or transplanted into a patient. These antibodies and antigen-binding fragments may be used alone or in association with other immunostimulatory molecules, e.g., other antibodies, fusion proteins, or small molecules including synergistic combination therapies.

An “immunomodulatory” antibody or antigen-binding fragment according to the invention also includes an “immunoinhibitory antibody” or antigen-binding fragment that specifically binds VSTM5. An “immunoinhibitory antibody” or “immunoinhibitory VSTM5 targeting antibody” or “immunoinhibitory VSTM5 specific antibody”, used herein interchangeably, includes any antibody which agonizes at least one effect of VSTM5 on immunity, either in vivo or ex vivo. These immunoinhibitory effects may be obtained by in vivo administration of such immunoinhibitory antibodies and antigen-binding fragments or ex vivo, e.g., by contacting a patient cell sample or tissue or organ, e.g., bone marrow or stem cells, with an immunoinhibitory antibody or antigen-binding fragment according to the invention which is then infused, re-infused or transplanted into a treated subject. These antibodies are particularly useful for reducing or preventing undesirable immune responses that occur as a result of immune related diseases such as autoimmunity, inflammation and allergy and/or for reducing undesirable immune activation that may occur as the result of cell or gene therapy or tissue or organ transplant such as GVHD. For example such immunoinhibitory antibodies will agonize or potentiate at least one of the effects of VSTM5 on immune cells and immune responses such as the inhibition of pathogenic T cells and/or NK cells and/or the enhancement of the number and immune tolerizing effects of Treg cells, e.g., iTregs or myeloid derived suppressor cells (MDSCs).

Enhancement of or mimicking the immunoinhibitory activity of VSTM5 may especially be desirable in situations in which VSTM5 itself (or biological systems into which it feeds or in which it participates) is abnormally downregulated, and/or situations in which increased activity of VSTM5 is likely to have a beneficial effect, such as for example, treatment of conditions wherein immunity is abnormally upregulated and/or for reducing or preventing undesirable immune activation. As used herein, an “immunoinhibitory VSTM5 targeting antibody” may mimic or increase at least one of the effects or activity of VSTM5 on immunity and specific immune cells. Similarly, these immunoinhibitory antibodies or antigen-binding fragments may be used alone or in combination with other drugs or biologics, including other immunoinhibitory drugs or biologics, and especially combinations that may elicit a synergistic inhibitory effect on immunity, e.g., the inhibition of pathogenic T or NK cells.

The present invention includes, according to at least some embodiments, immunomodulatory antibodies that interact with one or more epitopes on the VSTM5 polypeptide, wherein such antibody or antigen-binding fragment inhibits or blocks (antagonizes), or mimics or promotes (agonizes) in vivo or ex vivo at least one of the effects of VSTM5 on immunity or on specific types of immune cells, e.g., T or NK cells. While the description herein provides non-limiting examples of antibodies that bind to discrete portions of VSTM5, the present invention, in at least some embodiments, provides means for identifying other immunomodulatory anti-VSTM5 antibodies and antigen-binding fragments, e.g., by screening a population of anti-VSTM5 antibodies or a phage or yeast library, hybridomas or cells or cell lines, or other cells or viruses which express such antibodies or antigen-binding fragments, for those of which potentiate or inhibit at least one effect of VSTM5 on immunity or on specific types of immune cells. In particular, a skilled artisan may conduct screening assays in vitro or in vivo such as described herein in order to determine whether a specific anti-VSTM5 antibody or antigen-binding fragment inhibits or potentiates the various effects of VSTM5 on immunity and on specific types of immune cells such as, e.g., the inhibitory effects of VSTM5 on CD4⁺ T cell activation or proliferation, CD8⁺ T (CTL) cell proliferation and/or CTL mediated cell depletion, NK cell activity and NK mediated cell depletion, the potentiating effects of VSTM5 on Treg cell differentiation and proliferation and Treg- or myeloid derived suppressor cell (MDSC)—mediated immunosuppression or immune tolerance, and/or the effects of VSTM5 on proinflammatory cytokine production by immune cells, e.g., IL-2, IFN-γ or TNF-α production by T or other immune cells. Preferably, such immunomodulatory antibodies and antigen-binding fragments will be suitable for use in human therapy, e.g., they will typically be human, chimeric, primatized or humanized antibodies or antigen-binding fragments and will generally possess a VSTM5 binding affinity and in vivo half-life appropriate for human therapy, e.g., for treating disease conditions such as cancer, infectious disease and chronic immune conditions such as autoimmunity, inflammatory diseases, allergic disorders and transplant recipients.

In specific exemplary embodiments the anti-VSTM5 immunomodulatory antibody or an antigen-binding fragment thereof comprises an antigen-binding region that binds specifically to a first polypeptide having an amino acid sequence set forth in any of SEQ ID NOs:1, 12-21, such that with regard to a second polypeptide that comprises to said first polypeptide, said second polypeptide having an amino acid sequence set forth in any of SEQ ID NOs: 2, 3, 6, 7, 132, 349, said antigen-binding region does not specifically bind or interact with any other portion of said second polypeptide apart from said first polypeptide.

With respect to the foregoing, SEQ ID NO:1 corresponds to amino acids 42-137 of SEQ ID NO: 6; SEQ ID NO:12 corresponds to amino acids 64-81 of SEQ ID NO: 6; SEQ ID NO:13 corresponds to amino acids 64-82 of SEQ ID NO: 6; SEQ ID NO:14 corresponds to amino acids 63-81 of SEQ ID NO: 6; SEQ ID NO:15 corresponds to amino acids 63-82 of SEQ ID NO: 6; SEQ ID NO:16 corresponds to amino acids 116-143 of SEQ ID NO: 6; SEQ ID NO:17 corresponds to amino acids 116-138 of SEQ ID NO: 6; SEQ ID NO:18 corresponds to amino acids 116-142 of SEQ ID NO: 6; SEQ ID NO:19 corresponds to amino acids 96-107 of SEQ ID NO: 6; SEQ ID NO:20 corresponds to amino acids 96-112 of SEQ ID NO: 6; and SEQ ID NO:21 corresponds to amino acids 97-108 of SEQ ID NO: 6.

Without wishing to be limited by a single hypothesis, VSTM5 polypeptides having the amino acid sequences of SEQ ID NOs 12-21 were predicted to comprise functional regions of the VSTM5 protein. These predictions were based on the analysis of a set of Protein Data Bank sequences (PDBs) which contained complexes of Ig proteins (for example PDB 1i85 which describe the complex of CTLA4 and CD86). The intermolecular contact residues from each PDB were collected and projected on the sequence of VSTM5. Several regions with clusters of interacting residues supported by several contact maps were identified and synthesized as a series of peptides with a potential to mimic the structure of the intact full length protein.

According to at least some embodiments, preferably the immunomodulatory antibody is a fully human antibody, chimeric antibody, humanized or primatized antibody or antigen-binding fragment thereof. These antibodies will typically comprise human constant regions or fragments thereof, e.g., IgG, IgA, IgD, IgM and IgE constant regions and most typically IgG1, IgG2, IgG3 and IgG4 constant regions. These constant regions optionally may be mutagenized or derivatized to enhance or inhibit specific antibody effector functions such as FcR binding, FcRn binding, ADCC activity, CDC activity, complement binding (e.g., C1q binding) and the like.

Additionally, in some instances the immunomodulatory antibody may optionally comprise or consist of a Fab, Fab′, F(ab′)2, F(ab′), F(ab), Fv or scFv fragment or minimal recognition unit which optionally may be conjugated to another moiety. This may be beneficial in treating sepsis as antibody fragments typically more rapidly desired sites, e.g. sites of infection, which may be beneficial or even essential in treating advanced sepsis.

Additionally, an immunomodulatory (immunostimulatory or immunoinhibitory) antibody according to at least some embodiments of the present invention may optionally be coupled to a therapeutic agent or a diagnostic agent such as a drug, a radionuclide, a fluorophore, an enzyme, a toxin, a therapeutic agent, or a chemotherapeutic agent; or a detectable marker such as a radioisotope, a metal chelator, an enzyme, a fluorescent compound, a bioluminescent compound or a chemiluminescent compound. Moreover, the subject antibodies may be coupled to other moieties such as water-soluble polymers (e.g., polyethylene glycol) which alter antibody half-life as well as other targeting moieties and other polypeptides including different antibodies or targeting moieties.

The invention, in at least some embodiments, further embraces pharmaceutical compositions comprising at least one immunomodulatory antibody or antigen-binding fragment or conjugate according to the invention and at least one pharmaceutically acceptable excipient or carrier.

In some embodiments the invention provides the use of immunomodulatory antibodies or antigen-binding fragments or pharmaceutical composition as described herein for treating subjects in need thereof, e.g. individuals diagnosed with diseases such as cancer, infectious conditions, sepsis, autoimmune conditions, inflammatory conditions, allergic conditions, or subjects have received or who are to receive cell or gene therapy, a transplanted tissue or organ, and other indications wherein upregulation or downregulation of immunity is desirable.

For example, the immunomodulatory antibody or antigen-binding fragment may be used to increase a subject's immune response against cancer or to potentiate the effect of another active agent or a cancer vaccine. Such cancer immunotherapy may be used as a monotherapy or may be combined with another therapeutic agent or therapy useful for treating cancer.

As another non-limiting example, combination therapy, i.e., treatment with an immunomodulatory antibody according to the invention and another therapeutic agent, e.g., a chemotherapeutic, biologic, radiation may convert non-responsive cancers to cancers that respond or better respond to immunotherapy or drug therapy. For example, in the case of a cancer that does not express a sufficient level of VSTM5 upon initial diagnosis prior to the initiation of the therapy (for the anti-VSTM5 antibody to be therapeutically beneficial) according to at least some embodiments of the present invention, VSTM5 expression may be induced by the therapy, or VSTM5 expression may increase on the subject's cancer, immune or stromal cells as the result of disease progression, thus making said cancer responsive to immunotherapy using VSTM5-specific antibodies, antibody fragments, conjugates and compositions comprising same. However it should be noted that in at least some embodiments, VSTM5 expression is not considered to be a prerequisite for successful treatment with an immunomodulatory antibody or antigen-binding fragment as described herein.

In particular, according to at least some embodiments the inventive immunomodulatory antibodies and antigen-binding fragments may be used in therapeutic regimens that include the use of one or more of radiotherapy, cryotherapy, antibody therapy, chemotherapy, photodynamic therapy, surgery, hormonal deprivation or combination therapy with conventional drugs as well as other immunomodulatory compounds such as small molecules, antibodies and fusion polypeptides.

For example, according to at least some embodiments such therapeutic agents may include by way of example cytotoxic drugs, tumor vaccines, antibodies, peptides, pepti-bodies, small molecules, chemotherapeutic agents, cytotoxic and cytostatic agents, immunological modifiers, interferons, interleukins, immunostimulatory growth hormones, cytokines, vitamins, minerals, aromatase inhibitors, RNAi, Histone Deacetylase Inhibitors, and proteasome inhibitors.

The inventive anti-VSTM5 antibodies and antigen-binding fragments and conjugates, and compositions containing same, according to at least some embodiments, may optionally be administered to a subject simultaneously or sequentially (in any order) with one or more other active agents or therapies such as radiotherapy, conventional/classical anti-cancer therapy potentiating anti-tumor immune responses, targeted therapy potentiating anti-tumor immune responses, therapeutic agents targeting Tregs and/or MDSCs, immunostimulatory antibodies, cytokine therapy, therapeutic cancer vaccines, adoptive cell transfer as well as other immunomodulatory compounds such as small molecules, antibodies and fusion polypeptides.

Conventional/classical anti-cancer agents include by way of example platinum based compounds, antibiotics with anti-cancer activity, Anthracyclines, Anthracenediones, alkylating agents, antimetabolites, Antimitotic agents, Taxanes, Taxoids, microtubule inhibitors, Folate antagonists and/or folic acid analogs, Topoisomerase inhibitors, Aromatase inhibitors, GnRh analogs, inhibitors of 5α-reductase, bisphosphonates; pyrimidine analogs, purine analogs and related inhibitors, vinca alkaloids, epipodophyllotoxins, antibiotics, L-Asparaginase, topoisomerase inhibitor, interferons, platinum coordination complexes, anthracenedione substituted urea, methyl hydrazine derivatives, adrenocortical suppressant, adrenocorticosteroids, progestins, estrogens, antiestrogen, androgens, antiandrogen, and gonadotropin-releasing hormone analog.

Specific but non-limiting examples of these categories of drugs are as follows: platinum based compounds such as oxaliplatin, cisplatin, carboplatin; Antibiotics with anti-cancer activity, such as dactinomycin, bleomycin, mitomycin-C, mithramycin and Anthracyclines, such as doxorubicin, daunorubicin, epirubicin, idarubicin; Anthracenediones, such as mitoxantrone; Alkylating agents, such as dacarbazine, melphalan, cyclophosphamide, temozolomide, chlorambucil, busulphan, nitrogen mustard, nitrosoureas; Antimetabolites, such as fluorouracil, raltitrexed, gemcitabine, cytosine arabinoside, hydroxyurea and Folate antagonists, such as methotrexate, trimethoprim, pyrimethamine, pemetrexed; Antimitotic agents such as polokinase inhibitors and Microtubule inhibitors, such as Taxanes and Taxoids, such as paclitaxel, docetaxel; Vinca alkaloids such as vincristine, vinblastine, vindesine, vinorelbine; Topoisomerase inhibitors, such as etoposide, teniposide, amsacrine, topotecan, irinotecan, camptothecin; Cytostatic agents including Antiestrogens such as tamoxifen, fulvestrant, toremifene, raloxifene, droloxifene, iodoxyfene, Antiandrogens such as bicalutamide, flutamide, nilutamide and cyproterone acetate, Progestogens such as megestrol acetate, Aromatase inhibitors such as anastrozole, letrozole, vorozole, exemestane; GnRH analogs, such as leuprorelin, goserelin, buserelin, degarelix; inhibitors of 5α-reductase such as finasteride.

More preferably, the chemotherapeutic agent is selected from the group consisting of 5-fluorouracil (5-FU), leucovorin (LV), irenotecan, oxaliplatin, capecitabine, paclitaxel and doxetaxel. Two or more chemotherapeutic agents can be used in a cocktail to be administered in combination with administration of the anti-VEGF antibody. One preferred combination chemotherapy is fluorouracil-based, comprising 5-FU and one or more other chemotherapeutic agent(s). Suitable dosing regimens of combination chemotherapies are known in the art and described in, for example, Saltz et al. (1999) Proc ASCO 18:233a and Douillard et al. (2000) Lancet 355:1041-7. The biologic may be another immune potentiators such as antibodies to PD-L1, PD-L2, CTLA-4, or VISTA as well as PD-L1, PD-L2, CTLA-4 or VISTA fusion proteins as well as cytokines, growth factor antagonists and agonists, hormones and anti-cytokine antibodies.

According to at least some embodiments of the invention, Targeted therapies used as agents for combination with anti VSTM5 antibodies for treatment of cancer are selected from the group consisting of but not limited to: histone deacetylase (HDAC) inhibitors, such as vorinostat, romidepsin, panobinostat, belinostat, mocetinostat, abexinostat, entinostat, resminostat, givinostat, quisinostat, sodium butyrate; Proteasome inhibitors, such as bortezomib, carfilzomib, disulfiram; mTOR pathway inhibitors, such as temsirolimus, rapamycin, everolimus; PI3K inhibitors, such as perifosine, CAL101, PX-866, IPI-145, BAY 80-6946; B-raf inhibitors such as vemurafenib, sorafenib; JAK2 inhibitors, such as lestaurtinib, pacritinib; Tyrosine kinase inhibitors (TKIs), such as erlotinib, imatinib, sunitinib, lapatinib, gefitinib, sorafenib, nilotinib, toceranib, bosutinib, neratinib, vatalanib, regorafenib, cabozantinib; other Protein kinase inhibitors, such as crizotinib; Inhibitors of serine/threonine kinases for example Ras/Raf signalling inhibitors such as farnesyl transferase inhibitors; Inhibitors of serine proteases for example matriptase, hepsin, urokinase; Inhibitors of intracellular signaling such as tipifarnib, perifosine; Inhibitors of cell signalling through MEK and/or AKT kinases; aurora kinase inhibitors such as AZD1152, PH739358, VX-680, MLN8054, R763, MP235, MP529, VX-528, AX39459; Cyclin dependent kinase inhibitors such as CDK2 and/or CDK4 inhibitors; Inhibitors of survival signaling proteins including Bcl-2, Bcl-XL, such as ABT-737; HSP90 inhibitors; Therapeutic monoclonal antibodies, such as anti-EGFR mAbs cetuximab, panitumumab, nimotuzumab, anti-ERBB2 mAbs trastuzumab, pertuzumab, anti-CD20 mAbs such as rituximab, ofatumumab, veltuzumab and mAbs targeting other tumor antigens such as alemtuzumab, labetuzumab, adecatumumab, oregovomab, onartuzumab; TRAIL pathway agonists, such as dulanermin (soluble rhTRAIL), apomab, mapatumumab, lexatumumab, conatumumab, tigatuzumab; Antibody fragments, bi-specific antibodies and bi-specific T-cell engagers (BiTEs), such as catumaxomab, blinatumomab; Antibody drug conjugates (ADC) and other immunoconjugates, such as ibritumomab triuxetan, tositumomab, brentuximab vedotin, gemtuzumab ozogamicin, clivatuzumab tetraxetan, pemtumomab, trastuzumab emtansine; Anti-angiogenic therapy such as bevacizumab, etaracizumab, volociximab, ramucirumab, aflibercept, sorafenib, sunitinib, regorafenib, axitinib, nintedanib, motesanib, pazopanib, cediranib; Metalloproteinase inhibitors such as marimastat; Inhibitors of urokinase plasminogen activator receptor function; Inhibitors of cathepsin activity.

Other therapeutic antibodies which may be used in combination with an immunomodulatory antibody according to the invention include by way of example cetuximab, panitumumab, nimotuzumab, trastuzumab, pertuzumab, rituximab, ofatumumab, veltuzumab, alemtuzumab, labetuzumab, adecatumumab, oregovomab, onartuzumab; apomab, mapatumumab, lexatumumab, conatumumab, tigatuzumab, catumaxomab, blinatumomab, ibritumomab triuxetan, tositumomab, brentuximab vedotin, gemtuzumab ozogamicin, clivatuzumab tetraxetan, pemtumomab, trastuzumab emtansine, bevacizumab, etaracizumab, volociximab, ramucirumab, aflibercept.

Therapeutic agent targeting immunosuppressive cells Tregs and/or MDSCs which may optionally be used in combination with an immunomodulatory antibody according to the at least some embodiments of the present invention include by way of example antimitotic drugs, cyclophosphamide, gemcitabine, mitoxantrone, fludarabine, thalidomide, thalidomide derivatives, COX-2 inhibitors, depleting or killing antibodies that directly target Tregs through recognition of Treg cell surface receptors, anti-CD25 daclizumab, basiliximab, ligand-directed toxins, denileukin diftitox (Ontak), a fusion protein of human IL-2 and diphtheria toxin, or LMB-2, a fusion between an scFv against CD25 and the pseudomonas exotoxin, antibodies targeting Treg cell surface receptors, TLR modulators, agents that interfere with the adenosinergic pathway, ectonucleotidase inhibitors, or inhibitors of the A2A adenosine receptor, TGF-β inhibitors, chemokine receptor inhibitors, retinoic acid, all-trans retinoic acid (ATRA), Vitamin D3, phosphodiesterase 5 inhibitors, sildenafil, ROS inhibitors and nitroaspirin.

Other immunostimulatory or immunoinhibitory antibodies which may according to at least some embodiments optionally be used in combination with an immunomodulatory antibody according to the invention include by way of example agonistic or antagonistic antibodies targeting one or more of CTLA4, PD-1, PDL-1, LAG-3, TIM-3, BTLA, B7-H4, B7-H3, VISTA, and/or agonistic or antagonistic antibodies targeting one or more of CD40, CD137, OX40, GITR, CD27, CD28 or ICOS, or fusion proteins containing any of the foregoing or fragments thereof which function as immune agonists or antagonists.

As described infra, without wishing to be limited by a single hypothesis, VSTM5 apparently interacts with a receptor expressed by NK cells. Accordingly, the subject immunomodulatory antibody or immunomodulatory antigen-binding fragments may be used on combination or coupled to an antibody or antigen-binding fragment thereof, or other moiety which specifically binds to an NK cell receptor. Such moieties which specifically bind to an NK cell receptor may agonize or antagonize the effect of said NK cell receptor. Various non-limiting examples are given herein. Such NK receptors include those of unknown function, as well as those known to inhibit NK cell activity such as KIR2DL1, KIR2DL2/3, KIR2DL4, KIR2DL5A, KIR2DL5B, KIR3DL1, KIR3DL2, KIR3DL3, LILRB1, NKG2A, NKG2C, NKG2E and LILRBS and those known to promote or activate NK cell activity such as NKp30, NKp44, NKp46, NKp46, NKG2D, KIR2DS4 CD2, CD16, CD69, DNAX accessory molecule-1 (DNAM-1), 2B4, NK1.1; a killer immunoglobulin (Ig)-like activating receptors (KAR); ILTs/LIRs; NKRP-1, CD69; CD94/NKG2C and CD94/NKG2E heterodimers, NKG2D homodimer KIR2DS and KIR3DS.

Therapeutic cancer vaccines may also be used in combination with an immunomodulatory antibody according to at least some embodiments of the invention, including but not limited to exogenous cancer and infectious agent vaccines including proteins or peptides used to mount an immunogenic response to a tumor antigen or an infectious agent, recombinant virus and bacteria vectors encoding tumor antigens, DNA-based vaccines encoding tumor antigens, proteins targeted to dendritic cells, dendritic cell-based vaccines, whole tumor cell vaccines, gene modified tumor cells expressing GM-CSF, ICOS and/or Flt3-ligand, oncolytic virus vaccines.

Cytokines which according to at least some embodiments may be used in combination with an immunomodulatory antibody according to the invention include by way of example one or more cytokines such as interferons, interleukins, colony stimulating factors, and tumor necrosis factors such as IL-2, IL-7, IL-12, IL-15, IL-17, IL-18, IL-21, IL-23, IL-27, GM-CSF, IFNα (interferon α), IFNα-2b, IFNβ, IFNγ, TNF-α, TNF-β and combinations thereof.

Adoptive cell transfer therapy according to at least some embodiments that may be used in combination with an immunomodulatory antibody according to the invention include by way of example an ex vivo treatment selected from expansion of the patient autologous naturally occurring tumor specific T cells or genetic modification of T cells to confer specificity for tumor antigens.

In some embodiments the invention provides the use of an immunostimulatory antibody, antigen-binding fragment or conjugate thereof according to the invention or a pharmaceutical composition containing, to perform one or more of the following in a subject in need thereof: (a) upregulating pro-inflammatory cytokines; (b) increasing T-cell proliferation and/or expansion; (c) increasing interferon-γ or TNF-α production by T-cells; (d) increasing IL-2 secretion; (e) stimulating antibody responses; (f) inhibiting cancer cell growth; (g) promoting antigenic specific T cell immunity; (h) promoting CD4⁺ and/or CD8⁺ T cell activation; (i) alleviating T-cell suppression; (j) promoting NK cell activity; (k) promoting apoptosis or lysis of cancer cells; and/or (l) cytotoxic or cytostatic effect on cancer cells.

In other embodiments the invention provides the use of an immunoinhibitory antibody, antigen-binding fragment or conjugate thereof according to at least some embodiments of the invention (optionally in a pharmaceutical composition) to agonize at least one immune inhibitory effect of VSTM5.

Such an antibody, antigen-binding fragment or conjugate thereof optionally and preferably mediates at least one of the following effects: (i) decreases immune response, (ii) decreases T cell activation, (iii) decreases cytotoxic T cell activity, (iv) decreases natural killer (NK) cell activity, (v) decreases T-cell activity, (vi) decreases pro-inflammatory cytokine secretion, (vii) decreases IL-2 secretion; (viii) decreases interferon-γ production, (ix) decreases Th1 response, (x) decreases Th2 response, (xi) increases cell number and/or activity of regulatory T cells, (xii) increases regulatory cell activity and/or one or more of myeloid derived suppressor cells (MDSCs), iMCs, mesenchymal stromal cells, TIE2-expressing monocytes, (xiii) increases regulatory cell activity and/or the activity of one or more of myeloid derived suppressor cells (MDSCs), iMCs, mesenchymal stromal cells, TIE2-expressing monocytes, (xiii) increases M2 macrophages, (xiv) increases M2 macrophage activity, (xv) increases N2 neutrophils, (xvi) increases N2 neutrophils activity, (xvii) increases inhibition of T cell activation, (xviii) increases inhibition of CTL activation, (xix) increases inhibition of NK cell activation, (xx) increases T cell exhaustion, (xxi) decreases T cell response, (xxii) decreases activity of cytotoxic cells, (xxiii) reduces antigen-specific memory responses, (xxiv) inhibits apoptosis or lysis of cells, (xxv) decreases cytotoxic or cytostatic effect on cells, (xxvi) reduces direct killing of cells, (xxvii) decreases Th17 activity, and/or (xxviii) reduces complement dependent cytotoxicity and/or antibody dependent cell-mediated cytotoxicity, with the proviso that said antibody, antigen-binding fragment or conjugate thereof may elicit an opposite effect to one or more of (i)-(xxviii).

In some embodiments the invention provides the use of an immunomodulatory antibody, antigen-binding fragment or conjugate according to the invention for diagnosing a disease in a subject, or for aiding in the diagnosis of a disease, wherein the disease is selected from the group consisting of cancer or an autoimmune disease, wherein the diagnostic method is performed ex vivo, by contacting a tissue or other sample from the subject with the immune molecule or antibody as described herein ex vivo and detecting specific binding thereto.

In other embodiments the invention provides the use of an immunomodulatory antibody, antigen-binding fragment or conjugate according to the invention in diagnostic methods for diagnosing or aiding in the diagnosis of a disease in a subject, wherein the disease is selected from the group consisting of cancer, an autoimmune disease, an allergic disease, an inflammatory disease, or an infectious disease wherein the diagnostic method is performed in vivo, comprising administering the immune molecule or antibody as described herein to the subject, preferably labeled with a detectable agent such as a radionuclide, or fluorophore and detecting specific binding of the immunomodulatory antibody, antigen-binding fragment or conjugate as described herein to a tissue of the subject. Alternatively the method may optionally be performed in vitro in a sample taken from the subject.

Optionally such diagnostic method may be performed before concurrent or after administering an immunomodulatory antibody, antigen-binding fragment or conjugate or composition containing according to at least some embodiments of the invention.

Optionally the diagnostic use or method further comprises determining a VSTM5 level in a tissue of the subject before administering the immunomodulatory antibody, antigen-binding fragment or conjugate or composition containing according to the invention to the subject. In some embodiments the immunomodulatory antibody, antigen-binding fragment or conjugate or composition containing according to the invention is only administered to the subject if said VSTM5 level is at a threshold level deemed to be “sufficient” for the VSTM5 antibody to elicit a significant therapeutic benefit, e.g., it is expressed at higher than normal levels or it is expressed at detectable levels by the treated disease cells, e.g., specific types of cancer or immune or stromal cells at the site of the disease, or is expressed at a level that based on in vitro or in vivo studies indicates that the antibody is likely to elicit a significant therapeutic benefit.

In some embodiments the expression level of VSTM5 is detected upon initial diagnosis prior to the initiation of cancer therapy, or alternatively after the start of cancer therapy, such as a combination therapy including use of an immunomodulatory antibody, antigen-binding fragment or conjugate according to the invention and another active such as a chemotherapeutic, therapeutic enzyme, radionuclide or radiation or another biologic.

In some embodiments the use or method further comprises determining said VSTM5 level according to the expression level of said VSTM5.

In some embodiments the VSTM5 expression level is determined by use of an IHC (immunohistochemistry) assay or a gene expression assay in a subject's tissue sample.

In some embodiments said IHC assay may comprise determining if the level of VSTM5 expression is at least 1 on a scale of 0 to 3, e.g., in a tissue sample comprising cancer cells and/or immune infiltrate and/or on immune and/or on stromal cells.

In some embodiments VSTM5 level may be determined in a tissue by contacting the tissue with an immunomodulatory antibody, antigen-binding fragment or conjugate or composition containing according to the invention and detecting specific binding thereto.

In some embodiments the invention provides assays for diagnosing or aiding in the diagnosis of a disease in a tissue sample taken from a subject, comprising use of an immunomodulatory antibody, antigen-binding fragment or conjugate as described herein and at least one reagent for diagnosing a disease selected from the group consisting of cancer, autoimmune disease, infectious disease, sepsis, or for inhibiting an undesirable immune activation that follows gene therapy.

In some embodiments the invention provides the use of an anti-VSTM5 antibody, antigen-binding fragment or conjugate or composition containing according to the invention for screening for a disease or aiding in the diagnosis of a disease (particularly one involving immunosuppression), detecting a presence or a severity of a disease, providing prognosis of a disease, monitoring disease progression or relapse, as well as assessment of treatment efficacy and/or relapse of a disease, disorder or condition, as well as selecting a therapy and/or a treatment for a disease, optimization of a given therapy for a disease, monitoring the treatment of a disease, and/or predicting the suitability of a therapy for specific patients or subpopulations or determining the appropriate dosing of a therapeutic product in patients or subpopulations.

In a some embodiments, the invention provides an anti-VSTM5 antibody, antigen-binding fragment or conjugate or composition containing according to the invention, and/or uses thereof for treatment and/or diagnosis of cancer, wherein the cancer, and/or immune cells infiltrating the cancer, and/or stromal cells of the subject express VSTM5, e.g. prior to, or following cancer therapy, and wherein said cancer is e.g., selected from the group consisting of breast cancer, cervical cancer, ovary cancer, endometrial cancer, melanoma, uveal melanoma, bladder cancer, lung cancer, pancreatic cancer, colorectal cancer, prostate cancer, leukemia, acute lymphocytic leukemia, chronic lymphocytic leukemia, B-cell lymphoma, Burkitt's lymphoma, multiple myeloma, Non-Hodgkin's lymphoma, myeloid leukemia, acute myelogenous leukemia (AML), chronic myelogenous leukemia, thyroid cancer, thyroid follicular cancer, myelodysplastic syndrome (MDS), fibrosarcomas and rhabdomyosarcomas, teratocarcinoma, neuroblastoma, glioma, glioblastoma, benign tumor of the skin, keratoacanthomas, renal cancer, anaplastic large-cell lymphoma, esophageal cancer, follicular dendritic cell carcinoma, seminal vesicle tumor, epidermal carcinoma, spleen cancer, bladder cancer, head and neck cancer, stomach cancer, liver cancer, bone cancer, brain cancer, cancer of the retina, biliary cancer, small bowel cancer, salivary gland cancer, cancer of uterus, cancer of testicles, cancer of connective tissue, myelodysplasia, Waldenström's macroglobinaemia, nasopharyngeal, neuroendocrine cancer, mesothelioma, angiosarcoma, Kaposi's sarcoma, carcinoid, fallopian tube cancer, peritoneal cancer, papillary serous müllerian cancer, malignant ascites, gastrointestinal stromal tumor (GIST), Li-Fraumeni syndrome, Von Hippel-Lindau syndrome (VHL), and cancer of unknown origin either primary or metastatic, wherein such cancers may be non-metastatic, invasive, or metastatic.

In some embodiments, the invention provides an immunomodulatory antibody, antigen-binding fragment or conjugate or composition containing according to the invention, and/or uses thereof for treatment and/or diagnosis of cancer, e.g., an immunostimulatory antibody, wherein said cancer is selected from the group consisting of B-cell lymphoma, Burkitt's lymphoma, thyroid cancer, thyroid follicular cancer, myelodysplastic syndrome (MDS), fibrosarcomas and rhabdomyosarcomas, melanoma, uveal melanoma, teratocarcinoma, neuroblastoma, glioma, glioblastoma cancer, keratoacanthomas, anaplastic large-cell lymphoma, esophageal squamous cells carcinoma, hepatocellular carcinoma cancer, follicular dendritic cell carcinoma, muscle-invasive cancer, seminal vesicle tumor, epidermal carcinoma, cancer of the retina, biliary cancer, small bowel cancer, salivary gland cancer, cancer of connective tissue, myelodysplasia, Waldenström's macroglobinaemia, nasopharyngeal, neuroendocrine cancer, myelodysplastic syndrome, mesothelioma, angiosarcoma, Kaposi's sarcoma, carcinoid, esophagogastric, fallopian tube cancer, peritoneal cancer, papillary serous müllerian cancer, malignant ascites, gastrointestinal stromal tumor (GIST), Li-Fraumeni syndrome, Von Hippel-Lindau syndrome (VHL); and endometrial cancer.

In some embodiments the invention provides the use of immunomodulatory antibody, antigen-binding fragment or conjugate or composition containing according to the invention in treating and/or detecting or aiding in the diagnosis of cancers that express VSTM5 at levels higher than other cancers such as:

Breast carcinoma, preferably any of ductal-carcinoma, infiltrating ductal carcinoma, lobular carcinoma, mucinous adenocarcinoma, intra duct and invasive ductal carcinoma, and Scirrhous adenocarcinoma;

Colorectal adenocarcinoma, preferably any of Poorly to Well Differentiated invasive and noninvasive Adenocarcinoma, Poorly to Well Differentiated Adenocarcinoma of the cecum, Well to Poorly Differentiated Adenocarcinoma of the colon, Tubular adenocarcinoma, preferably Grade 2 Tubular adenocarcinoma of the ascending colon, colon adenocarcinoma Duke's stage C1, invasive adenocarcinoma, Adenocarcinoma of the rectum, preferably Grade 3 Adenocarcinoma of the rectum, Moderately Differentiated Adenocarcinoma of the rectum, and Moderately Differentiated Mucinous adenocarcinoma of the rectum;

Lung cancer, preferably any of Well to Poorly differentiated Non-small cell carcinoma, Squamous Cell Carcinoma, preferably well to poorly Differentiated Squamous Cell Carcinoma, keratinizing squamous cell carcinoma, adenocarcinoma, preferably poorly to well differentiated adenocarcinoma, large cell adenocarcinoma, Small cell lung cancer, preferably Small cell lung carcinoma, and more preferably undifferentiated Small cell lung carcinoma;

Prostate adenocarcinoma, preferably any of Adenocarcinoma Gleason Grade 6 to 9, Infiltrating adenocarcinoma, High grade prostatic intraepithelial neoplasia, and undifferentiated carcinoma;

Stomach adenocarcinoma, preferably moderately differentiated gastric adenocarcinoma;

Ovary carcinoma, preferably any of cystadenocarcinoma, serous papillary cystic carcinoma, Serous papillary cystic carcinoma, and Invasive serous papillary carcinoma;

Brain cancer, preferably any of Astrocytoma, with the proviso that it is not a grade 2 astrocytoma, preferably grade 4 Astrocytoma, and Glioblastoma multiforme;

Kidney carcinoma, preferably Clear cell renal cell carcinoma;

Liver cancer, preferably any of Hepatocellular carcinoma, preferably Low Grade hepatocellular carcinoma, Fibrolamellar Hepatocellular Carcinoma;

Lymphoma, preferably any of, Hodgkin's Lymphoma and High to low grade Non-Hodgkin's Lymphoma.

In some embodiments, the invention provides an immunomodulatory antibody, antigen-binding fragment or conjugate thereof, e.g., an immunostimulatory antibody, or a composition containing according to the invention, including pharmaceutical and diagnostic compositions, and/or uses thereof for treatment and/or diagnosis and/or aiding in the diagnosis of a condition, e.g., wherein said immune condition is selected from the group consisting of an immune condition such as an autoimmune disease, inflammatory disease, allergic condition, or comprises gene or cell therapy, transplant rejection, or graft versus host disease.

Autoimmune, allergic and inflammatory conditions treatable or diagnosable using an immunomodulatory antibody, antigen-binding fragment or conjugate of the invention include but are not limited to autoimmune diseases and chronic inflammatory conditions. Moreover, when referring to specific autoimmune or chronic inflammatory conditions this is intended to include related conditions, e.g., as set forth in the definitions of specific autoimmune and inflammatory conditions infra. Non-limiting examples of such conditions which may be treated or diagnosed according to the invention include conditions such as: multiple sclerosis, including relapsing-remitting multiple sclerosis, primary progressive multiple sclerosis, and secondary progressive multiple sclerosis; psoriasis; rheumatoid arthritis; psoriatic arthritis, systemic lupus erythematosus (SLE); discoid lupus erythematosus, inflammatory bowel disease, ulcerative colitis; Crohn's disease; benign lymphocytic angiitis, thrombocytopenic purpura, idiopathic thrombocytopenia, idiopathic autoimmune hemolytic anemia, pure red cell aplasia, Sjögren's syndrome, rheumatic disease, connective tissue disease, inflammatory rheumatism, degenerative rheumatism, extra-articular rheumatism, juvenile rheumatoid arthritis, arthritis uratica, muscular rheumatism, chronic polyarthritis, cryoglobulinemic vasculitis, ANCA-associated vasculitis, antiphospholipid syndrome, myasthenia gravis, autoimmune haemolytica anemia, Guillain-Barré syndrome, chronic immune polyneuropathy, autoimmune thyroiditis, insulin dependent diabetes mellitus, type I diabetes, Addison's disease, membranous glomerulonephropathy, Goodpasture's disease, autoimmune gastritis, autoimmune atrophic gastritis, pernicious anemia, pemphigus, pemphigus vulgaris, cirrhosis, primary biliary cirrhosis, dermatomyositis, polymyositis, fibromyositis, myogelosis, celiac disease, immunoglobulin A nephropathy, Henoch-Schönlein purpura, Evans syndrome, Dermatitis, atopic dermatitis, psoriasis, psoriasis arthropathica, Graves' ophthalmopathy, scleroderma, systemic scleroderma, progressive systemic scleroderma, asthma, allergy, primary biliary cirrhosis, Hashimoto's thyroiditis, primary myxedema, sympathetic ophthalmia, autoimmune uveitis, hepatitis, chronic action hepatitis, collagen diseases, ankylosing spondylitis, periarthritis humeroscapularis, panarteritis nodosa, chondrocalcinosis, Wegener's granulomatosis, microscopic polyangiitis, chronic urticaria, bullous skin disorders, pemphigoid, atopic eczema, bullous pemphigoid, cicatricial pemphigoid, vitiligo, atopic eczema, eczema, chronic urticaria, autoimmune urticaria, normocomplementemic urticarial vasculitis, hypocomplementemic urticarial vasculitis, autoimmune lymphoproliferative syndrome, Devic's disease, sarcoidosis, pernicious anemia, childhood autoimmune hemolytic anemia, idiopathic autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, gingivitis, periodontitis, idiopathic pericarditis, pancreatitis, myocarditis, vasculitis, gastritis, gout, gouty arthritis, and inflammatory skin disorders, selected from the group consisting of psoriasis, atopic dermatitis, eczema, rosacea, urticaria, and acne, normocomplementemic urticarial vasculitis, pericarditis, myositis, anti-synthetase syndrome, scleritis, macrophage activation syndrome, Behçet's Syndrome, PAPA Syndrome, Blau's Syndrome, gout, adult and juvenile Still's disease, cryropyrinopathy, Muckle-Wells syndrome, familial cold-induced auto-inflammatory syndrome, neonatal onset multisystemic inflammatory disease, familial Mediterranean fever, chronic infantile neurologic, cutaneous and articular syndrome, a rheumatic disease, polymyalgia rheumatica, mixed connective tissue disease, inflammatory rheumatism, degenerative rheumatism, extra-articular rheumatism, juvenile arthritis, juvenile rheumatoid arthritis, systemic juvenile idiopathic arthritis, arthritis uratica, muscular rheumatism, chronic polyarthritis, reactive arthritis, Reiter's syndrome, rheumatic fever, relapsing polychondritis, Raynaud's phenomenon, vasculitis, cryoglobulinemic vasculitis, temporal arteritis, giant cell arteritis, Takayasu arteritis, Behcet's disease, chronic inflammatory demyelinating polyneuropathy, autoimmune thyroiditis, insulin dependent diabetes mellitus, type I diabetes, Addison's disease, membranous glomerulonephropathy, polyglandular autoimmune syndromes, Goodpasture's disease, autoimmune gastritis, autoimmune atrophic gastritis, pernicious anemia, pemphigus, pemphigus vulgaris, cirrhosis, primary biliary cirrhosis, idiopathic pulmonary fibrosis, myositis, dermatomyositis, juvenile dermatomyositis, polymyositis, fibromyositis, myogelosis, celiac disease, celiac sprue dermatitis, immunoglobulin A nephropathy, Henoch-Schonlein purpura, Evans syndrome, atopic dermatitis, psoriasis, psoriasis vulgaris, psoriasis arthropathica, Graves' disease, Graves' ophthalmopathy, scleroderma, systemic scleroderma, progressive systemic scleroderma, diffuse scleroderma, localized scleroderma, Crest syndrome, asthma, allergic asthma, allergy, primary biliary cirrhosis, fibromyalgia, chronic fatigue and immune dysfunction syndrome (CFIDS), autoimmune inner ear disease, Hyper IgD syndrome, Schnitzler's syndrome, autoimmune retinopathy, age-related macular degeneration, atherosclerosis, chronic prostatitis, alopecia, alopecia areata, alopecia universalis, alopecia totalis, autoimmune thrombocytopenic purpura, idiopathic thrombocytopenic purpura, pure red cell aplasia, and TNF receptor-associated periodic syndrome (TRAPS).

Exemplary autoimmune or inflammatory diseases which may be detected or treated using an immunomodulatory antibody, antigen-binding fragment or conjugate or composition containing according to at least some embodiments of the invention include but are not limited to multiple sclerosis, relapsing-remitting multiple sclerosis, primary progressive multiple sclerosis, secondary progressive multiple sclerosis; progressive relapsing multiple sclerosis, chronic progressive multiple sclerosis, transitional/progressive multiple sclerosis, rapidly worsening multiple sclerosis, clinically-definite multiple sclerosis, malignant multiple sclerosis, also known as Marburg's Variant, acute multiple sclerosis, conditions relating to multiple sclerosis such as benign multiple sclerosis, relapsing remitting multiple sclerosis, secondary progressive multiple sclerosis, primary progressive multiple sclerosis, progressive relapsing multiple sclerosis, chronic progressive multiple sclerosis, transitional/progressive multiple sclerosis, rapidly worsening multiple sclerosis, clinically-definite multiple sclerosis, malignant multiple sclerosis, also known as Marburg's Variant, and acute multiple sclerosis. In some embodiments “conditions relating to multiple sclerosis” include, e.g., Devic's disease, also known as Neuromyelitis Optica; acute disseminated encephalomyelitis, acute demyelinating optic neuritis, demyelinative transverse myelitis, Miller-Fisher syndrome, encephalomyeloradiculoneuropathy, acute demyelinative polyneuropathy, tumefactive multiple sclerosis and Balo's concentric sclerosis, psoriatic arthritis, gout and pseudo-gout, juvenile idiopathic arthritis, Still's disease, rheumatoid vasculitis, conditions relating to rheumatoid arthritis such as rheumatoid arthritis, gout and pseudo-gout, juvenile idiopathic arthritis, juvenile rheumatoid arthritis, Still's disease, ankylosing spondylitis, rheumatoid vasculitis, as well as other conditions relating to rheumatoid arthritis such as e.g., osteoarthritis, sarcoidosis, Henoch-Schönlein purpura, Psoriatic arthritis, Reactive arthritis, Spondyloarthropathy, septic arthritis, Hemochromatosis, Hepatitis, vasculitis, Wegener's granulomatosis, Lyme disease, Familial Mediterranean fever, Hyperimmunoglobulinemia D with recurrent fever, TNF receptor associated periodic syndrome, and Enteropathic arthritis associated with inflammatory bowel disease, discoid lupus, lupus arthritis, lupus pneumonitis, lupus nephritis, and conditions relating to systemic lupus erythematosus such as osteoarticular tuberculosis, antiphospholipid antibody syndrome, inflammation of various parts of the heart, such as pericarditis, myocarditis, and endocarditis, Lung and pleura inflammation, pleuritis, pleural effusion, chronic diffuse interstitial lung disease, pulmonary hypertension, pulmonary emboli, pulmonary hemorrhage, and shrinking lung syndrome, lupus headache, Guillain-Barré syndrome, aseptic meningitis, demyelinating syndrome, mononeuropathy, mononeuritis multiplex, myelopathy, cranial neuropathy, polyneuropathy, vasculitis, Collagenous colitis, Lymphocytic colitis, Ischemic colitis, Diversion colitis, Behçet's disease, Indeterminate colitis, thrombocytopenic purpura, idiopathic autoimmune hemolytic anemia, pure red cell aplasia, cryoglobulinemic vasculitis, ANCA-associated vasculitis, antiphospholipid syndrome, autoimmune haemolytica anemia, Guillain-Barré syndrome, chronic immune polyneuropathy, autoimmune thyroiditis, idiopathic diabetes, juvenile type 1 diabetes, maturity onset diabetes of the young, latent autoimmune diabetes in adults, gestational diabetes, conditions relating to type 1 diabetes such as one or more of type 1 diabetes, insulin-dependent diabetes mellitus, idiopathic diabetes, juvenile type 1 diabetes, maturity onset diabetes of the young, latent autoimmune diabetes in adults, gestational diabetes. Conditions relating to type 1 diabetes include, neuropathy including polyneuropathy, mononeuropathy, peripheral neuropathy and autonomicneuropathy; eye complications: glaucoma, cataracts, and retinopathy, membranous glomerulonephropathy, autoimmune gastritis, pemphigus vulgaris, cirrhosis, fibromyositis, celiac disease, immunoglobulin A nephropathy, Henoch-Schönlein purpura, Evans syndrome, atopic dermatitis, psoriasis, Graves' ophthalmopathy, systemic scleroderma, asthma, allergy, anterior uveitis (or iridocyclitis), intermediate uveitis (pars planitis), posterior uveitis (or chorioretinitis), panuveitic form, hepatitis, Wegener's granulomatosis, microscopic polyangiitis, chronic urticaria, bullous skin disorders, pemphigoid, atopic eczema, Devic's disease, childhood autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, gingivitis, periodontitis, pancreatitis, myocarditis, vasculitis, gastritis, gout, gouty arthritis, and inflammatory skin disorders, selected from the group consisting of psoriasis, Nonpustular Psoriasis including Psoriasis vulgaris and Psoriatic erythroderma (erythrodermic psoriasis), Pustular psoriasis including Generalized pustular psoriasis (pustular psoriasis of von Zumbusch), Pustulosis palmaris et plantaris (persistent palmoplantar pustulosis, pustular psoriasis of the Barber type, pustular psoriasis of the extremities), Annular pustular psoriasis, Acrodermatitis continua, Impetigo herpetiformis, drug-induced psoriasis, Inverse psoriasis, Napkin psoriasis, Seborrheic-like psoriasis, Guttate psoriasis, Nail psoriasis, Psoriatic arthritis, atopic dermatitis, eczema, rosacea, urticaria, and acne, normocomplementemic urticarial vasculitis, pericarditis, anti-synthetase syndrome, scleritis, macrophage activation syndrome, Behçet's Syndrome, PAPA Syndrome, Blau's Syndrome, gout, adult and juvenile Still's disease, cryropyrinopathy, Muckle-Wells syndrome, familial cold-induced auto-inflammatory syndrome, neonatal onset multisystemic inflammatory disease, familial Mediterranean fever, chronic infantile neurologic, cutaneous and articular syndrome, systemic juvenile idiopathic arthritis, Hyper IgD syndrome, Schnitzler's syndrome, autoimmune retinopathy, age-related macular degeneration, atherosclerosis, chronic prostatitis and TNF receptor-associated periodic syndrome (TRAPS).

“Inflammatory bowel disease” herein comprises any inflammatory bowel condition and especially includes inflammatory bowel disease, Crohn's disease, ulcerative colitis (UC), collagenous colitis, lymphocytic colitis, ischemic colitis, diversion colitis, Behçet's disease, and indeterminate colitis.

“Inflammatory disorders”, “inflammatory conditions” and/or “inflammation”, used interchangeably herein, refers broadly to chronic or acute inflammatory diseases, and expressly includes inflammatory autoimmune diseases and inflammatory allergic conditions. These conditions include by way of example inflammatory abnormalities characterized by dysregulated immune response to harmful stimuli, such as pathogens, damaged cells, or irritants. Inflammatory disorders underlie a vast variety of human diseases. Non-immune diseases with etiological origins in inflammatory processes include cancer, atherosclerosis, and ischemic heart disease. Examples of disorders associated with inflammation are described above.

According to at least some embodiments autoimmune diseases that may be treated or detected using an immunomodulatory antibody, antigen-binding fragment or conjugate or composition according to the invention include any of the types and subtypes of any of multiple sclerosis, rheumatoid arthritis, type I diabetes, psoriasis, systemic lupus erythematosus, inflammatory bowel disease, uveitis, or Sjögren's syndrome and related diseases and conditions as set forth in the Definitions infra.

As mentioned, optionally and in some instances preferably the subject anti-VSTM5 antibody treatment methods may be combined with another moiety useful for treating the specific immune condition.

Optionally the treatment is combined with another moiety useful for treating immune related condition.

Optionally the moiety is selected from the group consisting of immunosuppressants such as corticosteroids, cyclosporin, cyclophosphamide, prednisone, azathioprine, methotrexate, rapamycin, tacrolimus, leflunomide or an analog thereof; mizoribine; mycophenolic acid; mycophenolate mofetil; 15-deoxyspergualine or an analog thereof; biological agents such as TNF-α blockers or antagonists, or any other biological agent targeting any inflammatory cytokine, nonsteroidal antiinflammatory drugs/Cox-2 inhibitors, hydroxychloroquine, sulphasalazopryine, gold salts, etanercept, infliximab, mycophenolate mofetil, basiliximab, atacicept, rituximab, cytoxan, interferon β-1a, interferon β-1b, glatiramer acetate, mitoxantrone hydrochloride, anakinra and/or other biologics and/or intravenous immunoglobulin (IVIG), interferons such as IFN-β-1a (REBIF®. AVONEX® and CINNOVEX®) and IFN-β-1b (BETASERON®); EXTAVIA®, BETAFERON®, ZIFERON®); glatiramer acetate (COPAXONE®), a polypeptide; natalizumab (TYSABRI®), mitoxantrone (NOVANTRONE®), a cytotoxic agent, a calcineurin inhibitor, e.g. cyclosporin A or FK506; an immunosuppressive macrolide, e.g. rapamycine or a derivative thereof; e.g. 40-O-(2-hydroxy)ethyl-rapamycin, a lymphocyte homing agent, e.g. FTY720 or an analog thereof, corticosteroids; cyclophosphamide; azathioprene; methotrexate; leflunomide or an analog thereof; mizoribine; mycophenolic acid; mycophenolate mofetil; 15-deoxyspergualine or an analog thereof; immunosuppressive monoclonal antibodies, e.g., monoclonal antibodies to leukocyte receptors, e.g., MHC, CD2, CD3, CD4, CD11a/CD18, CD7, CD25, CD27, B7, CD40, CD45, CD58, CD137, ICOS, CD150 (SLAM), OX40, 4-1BB or their ligands; or other immunomodulatory compounds, e.g. CTLA4-Ig (abatacept, ORENCIA®, belatacept), CD28-Ig, B7-H4-Ig, or other costimulatory agents, or adhesion molecule inhibitors, e.g. mAbs or low molecular weight inhibitors including LFA-1 antagonists, Selectin antagonists and VLA-4 antagonists, or another immunomodulatory agent.

Thus, treatment of multiple sclerosis using the agents according to at least some embodiments of the present invention may be combined with, for example, any known therapeutic agent or method for treating multiple sclerosis. Non-limiting examples of such known therapeutic agent or method for treating multiple sclerosis include interferon class, IFN-β-1a (REBIF®. AVONEX® and CINNOVEX®) and IFN-β-1b (BETASERON®, EXTAVIA®, BETAFERON®, ZIFERON®); glatiramer acetate (COPAXONE®), a polypeptide; natalizumab (TYSABRI®); and mitoxantrone (NOVANTRONE®), a cytotoxic agent, Fampridine (AMPYRA®). Other drugs include corticosteroids, methotrexate, cyclophosphamide, azathioprine, and intravenous immunoglobulin (IVIG), inosine, Ocrelizumab (R1594), Mylinax (Caldribine®), alemtuzumab (Campath), daclizumab (Zenapax®), Panaclar®/dimethyl fumarate (BG-12), Teriflunomide® (HMR1726), fingolimod (FTY720), Laquinimod® (ABR216062), as well as Haematopoietic stem cell transplantation, NeuroVax®, Rituximab (Rituxan®) BCG vaccine, low dose naltrexone, helminthic therapy, angioplasty, venous stents, and alternative therapy, such as vitamin D, polyunsaturated fats, medical marijuana.

Thus, treatment of rheumatoid arthritis, using the agents according to at least some embodiments of the present invention may be combined with, for example, any known therapeutic agent or method for treating rheumatoid arthritis. Non-limiting examples of such known therapeutic agents or methods for treating rheumatoid arthritis include glucocorticoids, nonsteroidal anti-inflammatory drug (NSAID) such as salicylates, or cyclooxygenase-2 inhibitors, ibuprofen and naproxen, diclofenac, indomethacin, etodolac Disease-modifying antirheumatic drugs (DMARDs)—Oral DMARDs: Auranofin (Ridaura), Azathioprine (Imuran®), Cyclosporine (Sandimmune®, Gengraf®, Neoral®, generic), D-Penicillamine (Cuprimine®), Hydroxychloroquine (Plaquenil®), IM gold Gold sodium thiomalate (Myochrysine®) Aurothioglucose (Solganal®), Leflunomide (Arava®), Methotrexate (Rheumatrex®), Minocycline (Minocin®), Staphylococcal protein A immunoadsorption (Prosorba column), Sulfasalazine (Azulfidine®). Biologic DMARDs: TNF-α blockers including Adalimumab (Humira®), Etanercept (Enbrel®), Infliximab (Remicade®), golimumab (Simponi®), Certolizumab pegol (Cimzia®), and other Biological DMARDs, such as Anakinra (Kineret®), Rituximab (Rituxan®), Tocilizumab (Actemra®), CD28 inhibitor including Abatacept (Orencia®) and Belatacept.

Thus, treatment of IBD, using the agents according to at least some embodiments of the present invention may be combined with, for example, any known therapeutic agent or method for treating IBD. Non-limiting examples of such known therapeutic agents or methods for treating IBD include immunosuppression to control the symptom, such as prednisone, Mesalazine (including Asacol®, Pentasa®, Lialda®, Aspiro®), azathioprine (Imuran®), methotrexate, or 6-mercaptopurine, steroids, Ondansetron®, TNF-α blockers (including infliximab, adalimumab golimumab, Certolizumab pegol), Orencia (abatacept), ustekinumab (Stelara®), Briakinumab (ABT-874), Certolizumab pegol (Cimzia®), ITF2357 (Givinostat®), Natalizumab (Tysabri®), Firategrast® (SB-683699), Remicade® (infliximab), vedolizumab (MLN0002), other drugs including GSK1605786 CCX282-B (Traficet-EN®), AJM300, (ustekinumab), Semapimod® (CNI-1493) tasocitinib (CP-690550), LMW Heparin MMX, Budesonide MMX®, Simponi® (golimumab), MultiStem®, Gardasil® HPV vaccine, Epaxal Berna® (virosomal hepatitis A vaccine), surgery, such as bowel resection, strictureplasty or a temporary or permanent colostomy or ileostomy; antifungal drugs such as nystatin (a broad spectrum gut antifungal) and either itraconazole (Sporanox®) or fluconazole (Diflucan®); alternative medicine, prebiotics and probiotics, cannabis, Helminthic therapy or ova of the Trichuris suis helminth.

Thus, treatment of psoriasis, using the agents according to at least some embodiments of the present invention may be combined with, for example, any known therapeutic agent or method for treating psoriasis. Non-limiting examples of such known therapeutics for treating psoriasis include topical agents, typically used for mild disease, phototherapy for moderate disease, and systemic agents for severe disease. Non-limiting examples of topical agents: bath solutions and moisturizers, mineral oil, and petroleum jelly; ointment and creams containing coal tar, dithranol (anthralin), corticosteroids like desoximetasone (Topicort®), Betamethasone, fluocinonide, vitamin D3 analogues (for example, calcipotriol), and retinoids. Non-limiting examples of phototherapy: sunlight; wavelengths of 311-313 nm, psoralen and ultraviolet A phototherapy (PUVA). Non-limiting examples of systemic agents: Biologics, such as interleukin antagonists, TNF-α blockers including antibodies such as infliximab (Remicade), adalimumab (Humira), golimumab, certolizumab pegol, and recombinant TNF-α decoy receptor, etanercept (Enbrel); drugs that target T cells, such as efalizumab (Xannelim/Raptiva®), alefacept (Ameviv®), dendritic cells such Efalizumab; monoclonal antibodies (MAbs) targeting cytokines, including anti-IL-12/IL-23 (ustekinumab (brand name Stelara®)) and anti-Interleukin-17; Briakinumab (ABT-874); small molecules, including but not limited to ISA247; Immunosuppressants, such as methotrexate, cyclosporine; vitamin A and retinoids (synthetic forms of vitamin A); and alternative therapy, such as changes in diet and lifestyle, fasting periods, low energy diets and vegetarian diets, diets supplemented with fish oil rich in Vitamin A and Vitamin D (such as cod liver oil), Fish oils rich in the two omega-3 fatty acids eicosapentaenoic acid (EPA) and docosahexaenoic acid (DHA) and contain Vitamin E, Ichthyotherapy, Hypnotherapy, and cannabis.

Thus, treatment of type 1 diabetes, using the agents according to at least some embodiments of the present invention may be combined with, for example, any known therapeutic agent or method for treating type 1 diabetes. Non-limiting examples of such known therapeutics for treating type 1 diabetes include insulin, insulin analogs, islet transplantation, stem cell therapy including PROCHYMAL®, non-insulin therapies such as IL-1β inhibitors including Anakinra (Kineret®), Abatacept (Orencia®), Diamyd, alefacept (Ameviv®), Otelixizumab, DiaPep277 (Hsp60 derived peptide), a 1-Antitrypsin, Prednisone, azathioprine, Ciclosporin, E1-INT (an injectable islet neogenesis therapy comprising an epidermal growth factor analog and a gastrin analog), statins including Zocor®, Simlup®, Simcard®, Simvacor®, Sitagliptin® (dipeptidyl peptidase (DPP-4) inhibitor), Anti-CD3 mAb (e.g., Teplizumab); CTLA4-Ig (abatacept), Anti IL-1β (Canakinumab), Anti-CD20 mAb (e.g., rituximab).

Thus, treatment of uveitis, using the agents according to at least some embodiments of the present invention may be combined with, for example, any known therapeutic agent or method for treating uveitis. Non-limiting examples of such known therapeutics for treating uveitis include corticosteroids, topical cycloplegics, such as atropine or homatropine, or injection of PSTTA (posterior subtenon triamcinolone acetate), antimetabolite medications, such as methotrexate, TNF-α blockers (including infliximab, adalimumab, etanercept, golimumab, certolizumab pegol).

Thus, treatment for Sjögren's syndrome, using the agents according to at least some embodiments of the present invention may be combined with, for example, any known therapeutic agent or method for treating for Sjögren's syndrome. Non-limiting examples of such known therapeutics for treating for Sjögren's syndrome include Cyclosporine, pilocarpine (Salagen®) and cevimeline (Evoxac®), Hydroxychloroquine (Plaquenil®), cortisone (prednisone and others) and/or azathioprine (Imuran®) or cyclophosphamide (Cytoxan®), Dexamethasone, Thalidomide, Dehydroepiandrosterone, NGX267, Rebamipide®, FID 114657, Etanercept, Raptiva®, Belimumab, MabThera® (rituximab); Anakinra, intravenous immune globulin (IVIG), Allogeneic Mesenchymal Stem Cells (AlloMSC), Automatic neuro-electrostimulation by “Saliwell Crown”.

Thus, treatment for systemic lupus erythematosus, using the agents according to at least some embodiments of the present invention may be combined with, for example, any known therapeutic agent or method for treating for systemic lupus erythematosus. Non-limiting examples of such known therapeutics for treating for systemic lupus erythematosus include corticosteroids and Disease-modifying antirheumatic drugs (DMARDs), commonly anti-malarial drugs such as plaquenil and immunosuppressants (e.g. methotrexate and azathioprine) Hydroxychloroquine, cytotoxic drugs (e.g., cyclophosphamide and mycophenolate), Hydroxychloroquine (HCQ), Benlysta (belimumab), nonsteroidal anti-inflammatory drugs, Prednisone, Cellcept®, Prograf®, Atacicept®, Lupuzor®, Intravenous Immunoglobulins (IVIGs), CellCept® (mycophenolate mofetil), Orencia®, CTLA4-IgG4m (RG2077), rituximab, Ocrelizumab, Epratuzumab, CNTO 136, Sifalimumab (MEDI-545), A-623 (formerly AMG 623), AMG 557, Rontalizumab, paquinimod (ABR-215757), LY2127399, CEP-33457, Dehydroepiandrosterone, Levothyroxine, abetimus sodium (LIP 394), Memantine, Opiates, Rapamycin, Renal transplantation, stem cell transplantation.

In at least some embodiments, the invention provides a VSTM5-specific immunomodulatory antibody, antigen-binding fragment or conjugate or composition containing according to the invention, pharmaceutical compositions, and/or uses thereof for treatment and/or diagnosis of infectious disease, wherein said infectious disease is e.g., a disease caused by bacterium, virus, fungus or yeast, mycoplasm or a parasite or sepsis associated therewith.

As used herein the term “viral infection” comprises any infection caused by a virus, optionally including but not limited to Retroviridae (e.g., human immunodeficiency viruses, such as HIV-1 or HIV-2, acquired immune deficiency (AIDS) also referred to as HTLV-III, LAV or HTLV-III/LAV, or HIV-III; and other isolates, such as HIV-LP; Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever virus); Reoviridae (e.g., reoviruses, orbiviruses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes viruses); Poxviridae (variola viruses, vaccinia viruses, pox viruses); and Iridoviridae (e.g., African swine fever virus); and unclassified viruses (e.g., the etiological agents of Spongiform encephalopathies, the agent of delta hepatitides (thought to be a defective satellite of hepatitis B virus), the agents of non-A, non-B hepatitis (class 1—internally transmitted; class 2—parenterally transmitted (i.e., Hepatitis C); Norwalk and related viruses, and astroviruses) as well as Severe acute respiratory syndrome virus and respiratory syncytial virus (RSV).

As used herein the term “fungal infection” comprises any infection caused by a fungus, optionally including but not limited to Cryptococcus neoformans, Histoplasma capsulatum, Coccidioides immitis, Blastomyces dermatitidis, Chlamydia trachomatis, and Candida albicans.

As used herein the term “parasite infection” comprises any infection caused by a parasite, optionally including but not limited to protozoa, such as Amebae, Flagellates, Plasmodium falciparum, Toxoplasma gondii, Ciliates, Coccidia, Microsporidia, Sporozoa; helminthes, Nematodes (Roundworms), Cestodes (Tapeworms), Trematodes (Flukes), Arthropods, and aberrant proteins known as prions.

An infectious disorder and/or disease caused by bacteria may optionally comprise one or more of Sepsis, septic shock, sinusitis, skin infections, pneumonia, bronchitis, meningitis, Bacterial vaginosis, Urinary tract infection (UCI), Bacterial gastroenteritis, Impetigo and erysipelas, Erysipelas, Cellulitis, anthrax, whooping cough, lyme disease, Brucellosis, enteritis, acute enteritis, Tetanus, diphtheria, Pseudomembranous colitis, Gas gangrene, Acute food poisoning, Anaerobic cellulitis, Nosocomial infections, Diarrhea, Meningitis in infants, Traveller's diarrhea, Hemorrhagic colitis, Hemolytic-uremic syndrome, Tularemia, Peptic ulcer, Gastric and Duodenal ulcers, Legionnaire's Disease, Pontiac fever, Leptospirosis, Listeriosis, Leprosy (Hansen's disease), Tuberculosis, Gonorrhea, Ophthalmia neonatorum, Septic arthritis, Meningococcal disease including meningitis, Waterhouse-Friderichsen syndrome, Pseudomonas infection, Rocky mountain spotted fever, Typhoid fever type salmonellosis, Salmonellosis with gastroenteritis and enterocolitis, Bacillary dysentery/Shigellosis, Coagulase-positive staphylococcal infections: Localized skin infections including Diffuse skin infection (Impetigo), Deep localized infections, Acute infective endocarditis, Septicemia, Necrotizing pneumonia, Toxinoses such as Toxic shock syndrome and Staphylococcal food poisoning, Cystitis, Endometritis, Otitis media, Streptococcal pharyngitis, Scarlet fever, Rheumatic fever, Puerperal fever, Necrotizing fasciitis, Cholera, Plague (including Bubonic plague and Pneumonic plague), as well as any infection caused by a bacteria selected from but not limited to Helicobacter pyloris, Boreliai burgdorferi, Legionella pneumophila, Mycobacteria sps (e.g., M. tuberculosis, M. avium, M. Intracellulare, M. kansaii, M gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracia, corynebacterium diphtherias, corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidum, Treponema pertenue, Leptospira, and Actinomyces israelii.

Non limiting examples of infectious disorder and/or disease caused by virus is selected from the group consisting of but not limited to acquired immune deficiency (AIDS), West Nile encephalitis, coronavirus infection, rhinovirus infection, influenza, dengue, hemorrhagic fever; an otological infection; severe acute respiratory syndrome (SARS), acute febrile pharyngitis, pharyngoconjunctival fever, epidemic keratoconjunctivitis, infantile gastroenteritis, infectious mononucleosis, Burkitt lymphoma, acute hepatitis, chronic hepatitis, hepatic cirrhosis, hepatocellular carcinoma, primary HSV-1 infection, (gingivostomatitis in children, tonsillitis & pharyngitis in adults, keratoconjunctivitis), latent HSV-1 infection (herpes labialis, cold sores), aseptic meningitis, Cytomegalovirus infection, Cytomegalic inclusion disease, Kaposi sarcoma, Castleman disease, primary effusion lymphoma, influenza, measles, encephalitis, postinfectious encephalomyelitis, Mumps, hyperplastic epithelial lesions (common, flat, plantar and anogenital warts, laryngeal papillomas, epidermodysplasia verruciformis), croup, pneumonia, bronchiolitis, Poliomyelitis, Rabies, bronchiolitis, pneumonia, German measles, congenital rubella, Hemorrhagic Fever, Chickenpox, Dengue, Ebola infection, Echovirus infection, EBV infection, Fifth Disease, Filovirus, Flavivirus, Hand, foot & mouth disease, Herpes Zoster Virus (Shingles), Human Papilloma Virus Associated Epidermal Lesions, Lassa Fever, Lymphocytic choriomeningitis, Parainfluenza Virus Infection, Paramyxovirus, Parvovirus B19 Infection, Picornavirus, Poxviruses infection, Rotavirus diarrhea, Rubella, Rubeola, Varicella, Variola infection.

An infectious disorder and/or disease caused by fungi optionally includes but is not limited to Allergic bronchopulmonary aspergillosis, Aspergilloma, Aspergillosis, Basidiobolomycosis, Blastomycosis, Candidiasis, Chronic pulmonary aspergillosis, Chytridiomycosis, Coccidioidomycosis, Conidiobolomycosis, Covered smut (barley), Cryptococcosis, Dermatophyte, Dermatophytid, Dermatophytosis, Endothrix, Entomopathogenic fungus, Epizootic lymphangitis, Epizootic ulcerative syndrome, Esophageal candidiasis, Exothrix, Fungemia, Histoplasmosis, Lobomycosis, Massospora cicadina, Mycosis, Mycosphaerella fragariae, Myringomycosis, Paracoccidioidomycosis, Pathogenic fungi, Penicilliosis, Thousand cankers disease, Tinea, Zeaspora, Zygomycosis. Non limiting examples of infectious disorder and/or disease caused by parasites is selected from the group consisting of but not limited to Acanthamoeba, Amoebiasis, Ascariasis, Ancylostomiasis, Anisakiasis, Babesiosis, Balantidiasis, Baylisascariasis, Blastocystosis, Candiru, Chagas disease, Clonorchiasis, Cochliomyia, Coccidia, Chinese Liver Fluke Cryptosporidiosis, Dientamoebiasis, Diphyllobothriasis, Dioctophyme renalis infection, Dracunculiasis, Echinococcosis, Elephantiasis, Enterobiasis, Fascioliasis, Fasciolopsiasis, Filariasis, Giardiasis, Gnathostomiasis, Hymenolepiasis, Halzoun Syndrome, Isosporiasis, Katayama fever, Leishmaniasis, lymphatic filariasis, Malaria, Metagonimiasis, Myiasis, Onchocerciasis, Pediculosis, Primary amoebic meningoencephalitis, Parasitic pneumonia, Paragonimiasis, Scabies, Schistosomiasis, Sleeping sickness, Strongyloidiasis, Sparganosis, Rhinosporidiosis, River blindness, Taeniasis (cause of Cysticercosis), Toxocariasis, Toxoplasmosis, Trichinosis, Trichomoniasis, Trichuriasis, Trypanosomiasis, and Tapeworm infection.

Some optional but particular examples of infectious disease include a disease caused by any of hepatitis B, hepatitis C, infectious mononucleosis, EBV, cytomegalovirus, AIDS, HIV-1, HIV-2, tuberculosis, malaria and schistosomiasis.

The therapeutic agents and/or a pharmaceutical composition comprising same, as recited herein, can be administered in combination with one or more additional therapeutic agents used for treatment of bacterial infections, including, but not limited to, antibiotics including Aminoglycosides, Carbapenems, Cephalosporins, Macrolides, Lincosamides, Nitrofurans, penicillins, Polypeptides, Quinolones, Sulfonamides, Tetracyclines, drugs against mycobacteria including but not limited to Clofazimine, Cycloserine, Cycloserine, Rifabutin, Rifapentine, Streptomycin and other antibacterial drugs such as Chloramphenicol, Fosfomycin, Metronidazole, Mupirocin, and Tinidazole.

The therapeutic agents and/or a pharmaceutical composition comprising same, as recited herein, can be administered in combination with one or more additional therapeutic agents used for treatment of viral infections, including, but not limited to, antiviral drugs such as oseltamivir (brand name Tamiflu®) and zanamivir (brand name Relenza®) Arbidol® —adamantane derivatives (Amantadine®, Rimantadine®)—neuraminidase inhibitors (Oseltamivir®, Laninamivir®, Peramivir®, Zanamivir®) nucleotide analog reverse transcriptase inhibitor including Purine analogue guanine (Aciclovir®/Valacyclovir®, Ganciclovir®/Valganciclovir®, Penciclovir®/Famciclovir®) and adenine (Vidarabine®), Pyrimidine analogue, uridine (Idoxuridine®, Trifluridine®, Edoxudine®), thymine (Brivudine®), cytosine (Cytarabine®); Foscarnet; Nucleoside analogues/NARTIs: Entecavir, Lamivudine®, Telbivudine®, Clevudine®; Nucleotide analogues/NtRTIs: Adefovir®, Tenofovir; Nucleic acid inhibitors such as Cidofovir®; InterferonInterferon alfa-2b, Peginterferon α-2a; Ribavirin®/Taribavirin®; antiretroviral drugs including zidovudine, lamivudine, abacavir, lopinavir, ritonavir, tenofovir/emtricitabine, efavirenz each of them alone or a various combinations, gp41 (Enfuvirtide), Raltegravir®, protease inhibitors such as Fosamprenavir®, Lopinavir® and Atazanavir®, Methisazone®, Docosanol®, Fomivirsen®, and Tromantadine®.

The therapeutic agents and/or a pharmaceutical composition comprising same, as recited herein, can be administered in combination with one or more additional therapeutic agents used for treatment of fungal infections, including, but not limited to, antifungal drugs of the Polyene antifungals, Imidazole, triazole, and thiazole antifungals, Allylamines, Echinocandins or other anti-fungal drugs.

Optionally the sepsis is selected from sepsis, severe sepsis, septic shock, systemic inflammatory response syndrome (SIRS), bacteremia, septicemia, toxemia, and septic syndrome.

Optionally the treatment is combined with another moiety useful for treating sepsis.

According to at least some embodiments there is provided a diagnostic method for determining whether to perform the use or to administer an antibody composition as described herein, comprising performing the diagnostic method as described herein.

In other embodiments the present invention relates to in vitro and animal screening assays for identifying antibodies and antigen-binding fragments that modulate (agonize or antagonize) at least one of the effects of VSTM5 on immune cells, cytokine production and immunity. For example, these assays may screen for anti-VSTM5 immunostimulatory antibodies, antigen-binding fragments or conjugates which suppress VSTM5 and thereby elicit one or more of the following effects on immunity (i) increases immune response, (ii) increases T cell activation, (iii) increases cytotoxic T cell activity, (iv) increases NK cell activity, (v) alleviates T-cell suppression, (vi) increases pro-inflammatory cytokine secretion, (vii) increases IL-2 secretion; (viii) increases interferon-γ production, (ix) increases Th1 response, (x) decrease Th2 response, (xi) decreases or eliminates cell number and/or activity of at least one of regulatory T cells (Tregs), myeloid derived suppressor cells (MDSCs), iMCs, mesenchymal stromal cells, TIE2-expressing monocytes, (xii) reduces regulatory cell activity, and/or the activity of one or more of myeloid derived suppressor cells (MDSCs), iMCs, mesenchymal stromal cells, TIE2-expressing monocytes, (xiii) decreases or eliminates M2 macrophages, (xiv) reduces M2 macrophage pro-tumorigenic activity, (xv) decreases or eliminates N2 neutrophils, (xvi) reduces N2 neutrophils pro-tumorigenic activity, (xvii) reduces inhibition of T cell activation, (xviii) reduces inhibition of CTL activation, (xix) reduces inhibition of NK cell activation, (xx) reverses T cell exhaustion, (xxi) increases T cell response, (xxii) increases activity of cytotoxic cells, (xxiii) stimulates antigen-specific memory responses, (xxiv) elicits apoptosis or lysis of cancer cells, (xxv) stimulates cytotoxic or cytostatic effect on cancer cells, (xxvi) induces direct killing of cancer cells, (xxvii) increases Th17 activity and/or (xxviii) induces complement dependent cytotoxicity and/or antibody dependent cell-mediated cytotoxicity, with the proviso that anti-VSTM5 antibody or antigen-binding fragment may elicit an opposite effect to one or more of (i)-(xxviii).

As an example only and without limitation, such anti-VSTM5 antibodies will be obtained by in vivo or in vitro immunization of an animal using VSTM5 or a fragment or conjugate thereof as an immunogen, e.g., a VSTM5-ECD-Ig fusion protein, optionally in combination with an adjuvant, or may be derived from phage or yeast antibody or Fab libraries. In such methods a population of antibodies or antibody or antibody fragment expressing cells, e.g., B cells, phage, yeast cells or hybridomas or recombinant cell lines, or other cells or viruses, that express these different antibodies will be screened to identify antibodies or antibody fragments that bind VSTM5 with sufficient avidity and these antibodies, antibody secreting cells, hybridomas or recombinant cell lines will further be screened to select for those anti-VSTM5 antibodies or antibody fragments that antagonize at least one of VSTM5's effect on immunity, e.g., T and NK cell immunity.

In other embodiments these assays may screen for anti-VSTM5 immunoinhibitory antibodies, antigen-binding fragments or conjugates which agonize or mimic the effects of VSTM5, and thereby, e.g., elicit one or more of the following effects on immunity (a) downregulate pro-inflammatory cytokines; (b) decrease T-cell proliferation and/or expansion; (c) decrease interferon-γ or TNF-α production by T-cells; (d) decrease IL-2 secretion; (e) reduce antibody responses; (f) suppress antigenic specific T cell immunity; (g) suppress CD4⁺ and/or CD8⁺ T cell activation; (h) increase T-cell suppression or TRegs and the induction of prolonged immunosuppression or tolerance; (i) reduce NK cell activity; and/or (j) suppress cytotoxic or cytostatic effect on cells.

Such anti-VSTM5 antibodies will be obtained by in vivo or in vitro immunization of an animal using VSTM5 or a fragment or conjugate thereof as an immunogen, e.g., a VSTM5-ECD-Ig fusion protein, optionally in combination with an adjuvant, or may be derived from phage or yeast antibody or Fab libraries. In such methods a population of antibodies or antibody or antibody fragment expressing cells, e.g., B cells, phage, yeast cells or hybridomas or recombinant cell lines, or other cells or viruses that express these different antibodies will be screened to identify antibodies or antibody fragments that bind VSTM5 with sufficient avidity and these antibodies, antibody secreting cells, hybridomas or recombinant cell lines will further be screened to select for those anti-VSTM5 antibodies or antibody fragments that agonize at least one of the suppressive effects of VSTM5 on immunity, e.g., its suppressive effect on T and NK cell immunity, and on the production of proinflammatory cytokines or its enhancing effect on Tregs.

Also, the invention provides immunomodulatory (immmunoinhibitory or immunstimulatory) antibodies and antigen-binding fragments identified by such screening assays, and variants thereof, e.g., chimeras, fragments and humanized, primatized and other variants thereof, in at least some embodiments.

According to at least some embodiments, there is provided an anti-VSTM5 antibody or an antigen-binding fragment thereof which specifically binds to the polypeptide of SEQ ID NO: 2, 3, 6, 7, 132, 349, or to a polypeptide possessing at least 90% sequence identity therewith or to a non-human VSTM5 ortholog, wherein such antibody or antigen-binding fragment either (1) enhances, agonizes or mimics, or (2) inhibits, antagonizes or blocks at least one effect that a VSTM5 polypeptide having the amino acid sequence of SEQ ID NO: 2, 3, 6, 7, 132, 349 elicits on immunity or on one or more types of immune cells.

According to at least some embodiments, there is provided an anti-VSTM5 antibody or an antigen-binding fragment thereof which comprises an antigen-binding region that binds specifically to (i) a first polypeptide having an amino acid sequence set forth in any of SEQ ID NOs:1, 12-21, or to a polypeptide possessing at least 90, 95, 96, 97, 98 or 99% sequence identity therewith or to the same region of a non-human VSTM5 ortholog, and (ii) wherein a second polypeptide having an amino acid sequence set forth in any of SEQ ID NOs: 2, 3, 6, 7, 132, 349 or a polypeptide possessing at least 90, 95, 96, 97, 98 or 99% sequence identity therewith or a non-human VSTM5 ortholog which comprises said first polypeptide, and (iii) with the further proviso that said antigen-binding region does not specifically bind to any other portion of said second polypeptide apart from said first polypeptide. Optionally said antibody or antigen binding fragment is an immunomodulatory antibody or an immunomodulatory antigen-binding fragment thereof.

According to at least some embodiments, there is provided an anti-VSTM5 antibody or antibody fragment that specifically competes for binding to human or murine VSTM5 with an anti-VSTM5 antibody or an antigen-binding fragment thereof selected from any of the specific anti-VSTM5 antibodies disclosed in this application or which binds the same epitope and/or which elicits the same immunomodulatory effects.

According to at least some embodiments, there is provided an anti-VSTM5 antibody or antibody fragment that comprises 1, 2, 3, 4, 5 or 6 of the CDRs and/or which elicits the same immunomodulatory effects as any of the specific anti-VSTM5 antibodies disclosed in this application.

According to at least some embodiments, there is provided an anti-VSTM5 antibody or antibody fragment that competes with an anti-VSTM5 antibody comprising a variable heavy (VH) region identical to that in SEQ ID NO: 253 and a variable light (VL) region identical to that in SEQ ID NO:254 for binding to human VSTM5 or a human VSTM5 fragment or a non-human VSTM5 ortholog and/or which elicits the same immunomodulatory effects as an anti-VSTM5 antibody comprising a variable heavy (VH) region identical to that in SEQ ID NO: 253 and a variable light (VL) region identical to that in SEQ ID NO:254. Optionally the anti-VSTM5 antibody or antibody fragment binds the same epitope as an anti-VSTM5 antibody comprising a variable heavy (VH) region identical to that in SEQ ID NO:253 and a variable light (VL) region identical to that in SEQ ID NO:254 and/or which elicits the same immunomodulatory effects.

According to at least some embodiments, there is provided an anti-VSTM5 antibody or antibody fragment that comprises a variable heavy (VH) region at least 90, 95, 96, 97, 98, or 99% identical to that in SEQ ID NO:253 and/or a variable light (VL) region at least 96, 97, 98, or 99% identical to that in SEQ ID NO:254.

According to at least some embodiments, there is provided an anti-VSTM5 antibody or antibody fragment that comprises a variable heavy (VH) region identical to that in SEQ ID NO: 253 and/or a variable light (VL) region identical to that in SEQ ID NO: 254.

According to at least some embodiments, there is provided an anti-VSTM5 antibody or antibody fragment that comprises a VH region containing 1, 2 or 3 of the CDRs of SEQ ID NO:253 and/or a VL region containing 1, 2 or 3 of the CDRs of SEQ ID NO:254.

According to at least some embodiments, there is provided an anti-VSTM5 antibody or antibody fragment that comprises a VH region containing CDR 1, 2 and 3 polypeptides having the sequences of SEQ ID NO:277, 278 and 279, or a sequence at least 90, 95, 96, 97, 98, or 99% identical thereto, and a VL region containing CDR 1, 2 and 3 polypeptides having the sequences of SEQ ID NO. 280, 281 and 282 or a sequence at least 90, 95, 96, 97, 98, or 99% identical thereto.

According to at least some embodiments, there is provided an anti-VSTM5 antibody or antibody fragment that competes for binding to human VSTM5 or to a human VSTM5 fragment or to a non-human VSTM5 ortholog as an anti-VSTM5 antibody comprising a variable heavy (VH) region identical to that in SEQ ID NO:255 and a variable light (VL) region identical to that in SEQ ID NO:256. Optionally the anti-VSTM5 antibody or antibody fragment binds the same epitope as an anti-VSTM5 antibody comprising a variable heavy (VH) region identical to that in SEQ ID NO:255 and a variable light (VL) region identical to that in SEQ ID NO:256 and/or which elicits the same immunomodulatory effects as an anti-VSTM5 antibody comprising a variable heavy (VH) region identical to that in SEQ ID NO:255 and a variable light (VL) region identical to that in SEQ ID NO:256.

According to at least some embodiments, there is provided an anti-VSTM5 antibody or antibody fragment that comprises a variable heavy (VH) region at least 90, 95, 96, 97, 98, or 99% identical to that in SEQ ID NO:255 and/or a variable light (VL) region at least 90, 95, 96, 97, 98, or 99% identical to that in SEQ ID NO:256.

According to at least some embodiments, there is provided an anti-VSTM5 antibody or antibody fragment that comprises a variable heavy (VH) region identical to that in SEQ ID NO: 255 and/or a variable light (VL) region identical to that in SEQ ID NO: 256.

According to at least some embodiments, there is provided an anti-VSTM5 antibody or antibody fragment that comprises a VH region containing 1, 2 or 3 of the CDRs of SEQ ID NO:255 and/or a VL region containing 1, 2 or 3 of the CDRs of SEQ ID NO:256.

According to at least some embodiments, there is provided an anti-VSTM5 antibody or antibody fragment that comprises a VH region containing CDR 1, 2 and 3 polypeptides having the sequences of SEQ ID NO:283, 284 and 285, or a sequence at least 90, 95, 96, 97, 98, or 99% identical thereto, and a VL region containing CDR 1, 2 and 3 polypeptides having the sequences of SEQ ID NO. 286, 287 and 288, or a sequence at least 90, 95, 96, 97, 98, or 99% identical thereto.

According to at least some embodiments, there is provided an anti-VSTM5 antibody or antibody fragment that competes for binding with an anti-VSTM5 antibody comprising a variable heavy (VH) region identical to that in SEQ ID NO:257 and a variable light (VL) region identical to that in SEQ ID NO:258 to human VSTM5 or to a human VSTM5 fragment or a non-human VSTM5 ortholog and/or which elicits the same immunomodulatory effects as an anti-VSTM5 antibody comprising a variable heavy (VH) region identical to that in SEQ ID NO:257 and a variable light (VL) region identical to that in SEQ ID NO:258.

Optionally the anti-VSTM5 antibody or antibody fragment binds the same epitope as an anti-VSTM5 antibody comprising a heavy (VH) region identical to that in SEQ ID NO:257 and a variable light (VL) region identical to that in SEQ ID NO:258 and/or which elicits the same immunomodulatory effects as an anti-VSTM5 antibody comprising a variable heavy (VH) region identical to that in SEQ ID NO:257 and a variable light (VL) region identical to that in SEQ ID NO:258.

According to at least some embodiments, there is provided an anti-VSTM5 antibody or antibody fragment that comprises a variable heavy (VH) region at least 90, 95, 96, 97, 98, or 99% identical to that in SEQ ID NO:257 and/or a variable light (VL) region at least 96, 97, 98, or 99% identical to that in SEQ ID NO:258.

According to at least some embodiments, there is provided an anti-VSTM5 antibody or antibody fragment that comprises a variable heavy (VH) region identical to that in SEQ ID NO: 257 and/or a variable light (VL) region identical to that in SEQ ID NO: 258.

According to at least some embodiments, there is provided an anti-VSTM5 antibody or antibody fragment that comprises a VH region containing 1, 2 or 3 of the CDRs of SEQ ID NO:257 and/or a VL region containing 1, 2 or 3 of the CDRs of SEQ ID NO:258.

According to at least some embodiments, there is provided an anti-VSTM5 antibody or antibody fragment that comprises a VH region containing CDR 1, 2 and 3 polypeptides having the sequences of SEQ ID NO:289, 290 and 291, or a sequence at least 90, 95, 96, 97, 98, or 99% identical thereto, and a VL region containing CDR 1, 2 and 3 polypeptides having the sequences of SEQ ID NO. 292, 293 and 294, or a sequence at least 90, 95, 96, 97, 98, or 99% identical thereto.

According to at least some embodiments, there is provided an anti-VSTM5 antibody or antibody fragment that competes for binding with an anti-VSTM5 antibody comprising a variable heavy (VH) region identical to that in SEQ ID NO:259 and a variable light (VL) region identical to that in SEQ ID NO:260 to human VSTM5 or a human VSTM5 fragment or to a non-human VSTM5 ortholog and/or which elicits the same immunomodulatory effects as an anti-VSTM5 antibody comprising a variable heavy (VH) region identical to that in SEQ ID NO:259 and a variable light (VL) region identical to that in SEQ ID NO:260. Optionally the anti-VSTM5 antibody or antibody fragment binds the same epitope as an anti-VSTM5 antibody comprising a variable heavy (VH) region identical to that in SEQ ID NO:259 and a variable light (VL) region identical to that in SEQ ID NO:260.

According to at least some embodiments, there is provided an anti-VSTM5 antibody or antibody fragment that comprises a variable heavy (VH) region at least 90, 95, 96, 97, 98, or 99% identical to that in SEQ ID NO:259 and/or a variable light (VL) region at least 96, 97, 98, or 99% identical to that in SEQ ID NO:260.

According to at least some embodiments, there is provided an anti-VSTM5 antibody or antibody fragment that comprises a variable heavy (VH) region identical to that in SEQ ID NO: 259 and/or a variable light (VL) region identical to that in SEQ ID NO: 260.

According to at least some embodiments, there is provided an anti-VSTM5 antibody or antibody fragment that comprises a VH region containing 1, 2 or 3 of the CDRs of SEQ ID NO: 259 and/or a VL region containing 1, 2 or 3 of the CDRs of SEQ ID NO:260.

According to at least some embodiments, there is provided an anti-VSTM5 antibody or antibody fragment that comprises a VH region containing CDR 1, 2 and 3 polypeptides having the sequences of SEQ ID NO:295, 296 and 297, or a sequence at least 90, 95, 96, 97, 98, or 99% identical thereto and a VL region containing CDR 1, 2 and 3 polypeptides having the sequences of SEQ ID NO. 298, 299 and 300, or a sequence at least 90, 95, 96, 97, 98, or 99% identical thereto.

According to at least some embodiments, there is provided an anti-VSTM5 antibody or antibody fragment that competes for binding with an anti-VSTM5 antibody comprising a variable heavy (VH) region identical to that in SEQ ID NO:261 and a variable light (VL) region identical to that in SEQ ID NO:262 to human VSTM5 or a human VSTM5 fragment or a non-human VSTM5 ortholog thereof and/or which elicits the same immunomodulatory effects as an anti-VSTM5 antibody comprising a variable heavy (VH) region identical to that in SEQ ID NO:261 and a variable light (VL) region identical to that in SEQ ID NO:262. Optionally the anti-VSTM5 antibody or antibody fragment binds the same epitope as an anti-VSTM5 antibody comprising a variable heavy (VH) region identical to that in SEQ ID NO:261 and a variable light (VL) region identical to that in SEQ ID NO:262 and/or which elicits the same immunomodulatory effects as an anti-VSTM5 antibody comprising a variable heavy (VH) region identical to that in SEQ ID NO:261 and a variable light (VL) region identical to that in SEQ ID NO:262.

According to at least some embodiments, there is provided an anti-VSTM5 antibody or antibody fragment that comprises a variable heavy (VH) region at least 90, 95, 96, 97, 98, or 99% identical to that in SEQ ID NO:261 and/or a variable light (VL) region at least 90, 95, 96, 97, 98, or 99% identical to that in SEQ ID NO:262.

According to at least some embodiments, there is provided an anti-VSTM5 antibody or antibody fragment that comprises a variable heavy (VH) region identical to that in SEQ ID NO: 261 and/or a variable light (VL) region identical to that in SEQ ID NO: 262.

According to at least some embodiments, there is provided an anti-VSTM5 antibody or antibody fragment that comprises a VH region containing 1, 2 or 3 of the CDRs of SEQ ID NO:261 and/or a VL region containing 1, 2 or 3 of the CDRs of SEQ ID NO:262.

According to at least some embodiments, there is provided an anti-VSTM5 antibody or antibody fragment that comprises a VH region containing CDR 1, 2 and 3 polypeptides having the sequences of SEQ ID NO:301, 302 and 303, or a sequence at least 90, 95, 96, 97, 98, or 99% identical thereto, and a VL region containing CDR 1, 2 and 3 polypeptides having the sequences of SEQ ID NO. 304, 305 and 306, or a sequence at least 90, 95, 96, 97, 98, or 99% identical thereto.

According to at least some embodiments, there is provided an anti-VSTM5 antibody or antibody fragment that competes for binding with an anti-VSTM5 antibody comprising a variable heavy (VH) region identical to that in SEQ ID NO:263 and a variable light (VL) region identical to that in SEQ ID NO:264 to human VSTM5 or a human VSTM5 fragment or a non-human VSTM5 ortholog thereof and/or which elicits the same immunomodulatory effects as an anti-VSTM5 antibody comprising a variable heavy (VH) region identical to that in SEQ ID NO:263 and a variable light (VL) region identical to that in SEQ ID NO:264. Optionally the anti-VSTM5 antibody or antibody fragment binds the same epitope as an anti-VSTM5 antibody comprising a variable heavy (VH) region identical to that in SEQ ID NO:263 and a variable light (VL) region identical to that in SEQ ID NO:264 and/or which elicits the same immunomodulatory effects as an anti-VSTM5 antibody comprising a variable heavy (VH) region identical to that in SEQ ID NO:263 and a variable light (VL) region identical to that in SEQ ID NO:264.

According to at least some embodiments, there is provided an anti-VSTM5 antibody or antibody fragment that comprises a variable heavy (VH) region at least 90, 95, 96, 97, 98, or 99% identical to that in SEQ ID NO:263 and/or a variable light (VL) region at least 90, 95, 96, 97, 98, or 99% identical to that in SEQ ID NO:264.

According to at least some embodiments, there is provided an anti-VSTM5 antibody or antibody fragment that comprises a variable heavy (VH) region identical to that in SEQ ID NO: 263 and/or a variable light (VL) region identical to that in SEQ ID NO: 264.

According to at least some embodiments, there is provided an anti-VSTM5 antibody or antibody fragment that comprises a VH region containing 1, 2 or 3 of the CDRs of SEQ ID NO:263 and/or a VL region containing 1, 2 or 3 of the CDRs of SEQ ID NO:264.

According to at least some embodiments, there is provided an anti-VSTM5 antibody or antibody fragment that comprises a VH region containing CDR 1, 2 and 3 polypeptides having the sequences of SEQ ID NO:307, 308 and 309, or a sequence at least 90, 95, 96, 97, 98, or 99% identical thereto, and a VL region containing CDR 1, 2 and 3 polypeptides having the sequences of SEQ ID NO. 310, 311 and 312, or a sequence at least 90, 95, 96, 97, 98, or 99% identical thereto.

According to at least some embodiments, there is provided an anti-VSTM5 antibody or antibody fragment that competes for binding with an anti-VSTM5 antibody or antigen binding fragment containing a variable heavy (VH) region identical to that in SEQ ID NO:265 and a variable light (VL) region identical to that in SEQ ID NO:266 to human VSTM5 or a human VSTM5 fragment or to a non-human VSTM5 ortholog and/or which elicits the same immunomodulatory effects as an anti-VSTM5 antibody comprising a variable heavy (VH) region identical to that in SEQ ID NO:265 and a variable light (VL) region identical to that in SEQ ID NO:266. Optionally the anti-VSTM5 antibody or antibody fragment binds the same epitope as an anti-VSTM5 antibody comprising a variable heavy (VH) region identical to that in SEQ ID NO:265 and a variable light (VL) region identical to that in SEQ ID NO:266 and/or which elicits the same immunomodulatory effects as an anti-VSTM5 antibody comprising a variable heavy (VH) region identical to that in SEQ ID NO:265 and a variable light (VL) region identical to that in SEQ ID NO:266.

According to at least some embodiments, there is provided an anti-VSTM5 antibody or antibody fragment that comprises a variable heavy (VH) region at least 90, 95, 96, 97, 98, or 99% identical to that in SEQ ID NO:265 and/or a variable light (VL) region at least 90, 95, 96, 97, 98, or 99% identical to that in SEQ ID NO:266.

According to at least some embodiments, there is provided an anti-VSTM5 antibody or antibody fragment that comprises a variable heavy (VH) region identical to that in SEQ ID NO: 265 and/or a variable light (VL) region identical to that in SEQ ID NO: 266.

According to at least some embodiments, there is provided an anti-VSTM5 antibody or antibody fragment that comprises a VH region containing 1, 2 or 3 of the CDRs of SEQ ID NO:265 and/or a VL region containing 1, 2 or 3 of the CDRs of SEQ ID NO:266.

According to at least some embodiments, there is provided an anti-VSTM5 antibody or antibody fragment that comprises a VH region containing CDR 1, 2 and 3 polypeptides having the sequences of SEQ ID NO:313, 314 and 315, or a sequence at least 90, 95, 96, 97, 98, or 99% identical thereto, and a VL region containing CDR 1, 2 and 3 polypeptides having the sequences of SEQ ID NO. 316, 317 and 318, or a sequence at least 90, 95, 96, 97, 98, or 99% identical thereto.

According to at least some embodiments, there is provided an anti-VSTM5 antibody or antibody fragment that competes for binding with an anti-VSTM5 a variable heavy (VH) region identical to that in SEQ ID NO:267 and a variable light (VL) region identical to that in SEQ ID NO:268 to human VSTM5 or a human VSTM5 fragment or to a non-human VSTM5 ortholog and/or which elicits the same immunomodulatory effects as an anti-VSTM5 antibody comprising a variable heavy (VH) region identical to that in SEQ ID NO:267 and a variable light (VL) region identical to that in SEQ ID NO:268. Optionally the anti-VSTM5 antibody or antibody fragment binds the same epitope as an anti-VSTM5 antibody comprising a variable heavy (VH) region identical to that in SEQ ID NO:267 and a variable light (VL) region identical to that in SEQ ID NO:268 and/or which elicits the same immunomodulatory effects as an anti-VSTM5 antibody comprising a variable heavy (VH) region identical to that in SEQ ID NO:267 and a variable light (VL) region identical to that in SEQ ID NO:268.

According to at least some embodiments, there is provided an anti-VSTM5 antibody or antibody fragment that comprises a variable heavy (VH) region at least 90, 95, 96, 97, 98, or 99% identical to that in SEQ ID NO:267 and/or a variable light (VL) region at least 90, 95, 96, 97, 98, or 99% identical to that in SEQ ID NO:268.

According to at least some embodiments, there is provided an anti-VSTM5 antibody or antibody fragment that comprises a variable heavy (VH) region identical to that in SEQ ID NO: 267 and/or a variable light (VL) region identical to that in SEQ ID NO: 268.

According to at least some embodiments, there is provided an anti-VSTM5 antibody or antibody fragment that comprises a VH region containing 1, 2 or 3 of the CDRs of SEQ ID NO:267 and/or a VL region containing 1, 2 or 3 of the CDRs of SEQ ID NO:268.

According to at least some embodiments, there is provided an anti-VSTM5 antibody or antibody fragment that comprises a VH region containing CDR 1, 2 and 3 polypeptides having the sequences of SEQ ID NO:319, 320 and 321, or a sequence at least 90, 95, 96, 97, 98, or 99% identical thereto, and a VL region containing CDR 1, 2 and 3 polypeptides having the sequences of SEQ ID NO. 322, 323 and 324, or a sequence at least 90, 95, 96, 97, 98, or 99% identical thereto.

According to at least some embodiments, there is provided an anti-VSTM5 antibody or antibody fragment that competes for binding with an anti-VSTM5 a variable heavy (VH) region identical to that in SEQ ID NO:269 and a variable light (VL) region identical to that in SEQ ID NO:270 to human VSTM5 or a human VSTM5 fragment or to a non-human VSTM5 ortholog and/or which elicits the same immunomodulatory effects as an anti-VSTM5 antibody comprising a variable heavy (VH) region identical to that in SEQ ID NO:269 and a variable light (VL) region identical to that in SEQ ID NO:270. Optionally the anti-VSTM5 antibody or antibody fragment binds the same epitope as an anti-VSTM5 antibody comprising a variable heavy (VH) region identical to that in SEQ ID NO:269 and a variable light (VL) region identical to that in SEQ ID NO:270 and/or which elicits the same immunomodulatory effects as an anti-VSTM5 antibody comprising a variable heavy (VH) region identical to that in SEQ ID NO:269 and a variable light (VL) region identical to that in SEQ ID NO:270.

According to at least some embodiments, there is provided an anti-VSTM5 antibody or antibody fragment that comprises a variable heavy (VH) region at least 90, 95, 96, 97, 98, or 99% identical to that in SEQ ID NO:269 and/or a variable light (VL) region at least 90, 95, 96, 97, 98, or 99% identical to that in SEQ ID NO:270.

According to at least some embodiments, there is provided an anti-VSTM5 antibody or antibody fragment that comprises a variable heavy (VH) region identical to that in SEQ ID NO: 269 and/or a variable light (VL) region identical to that in SEQ ID NO: 270.

According to at least some embodiments, there is provided an anti-VSTM5 antibody or antibody fragment that comprises a VH region containing 1, 2 or 3 of the CDRs of SEQ ID NO:269 and/or a VL region containing 1, 2 or 3 of the CDRs of SEQ ID NO:270.

According to at least some embodiments, there is provided an anti-VSTM5 antibody or antibody fragment that comprises a VH region containing CDR 1, 2 and 3 polypeptides having the sequences of SEQ ID NO:325, 326 and 327, or a sequence at least 90, 95, 96, 97, 98, or 99% identical thereto, and a VL region containing CDR 1, 2 and 3 polypeptides having the sequences of SEQ ID NO. 328, 329 and 330, or a sequence at least 90, 95, 96, 97, 98, or 99% identical thereto.

According to at least some embodiments, there is provided an anti-VSTM5 antibody or antibody fragment that competes for binding with an anti-VSTM5 a variable heavy (VH) region identical to that in SEQ ID NO:271 and a variable light (VL) region identical to that in SEQ ID NO:272 to human VSTM5 or a human VSTM5 fragment or to a non-human VSTM5 ortholog and/or which elicits the same immunomodulatory effects as an anti-VSTM5 antibody comprising a variable heavy (VH) region identical to that in SEQ ID NO:271 and a variable light (VL) region identical to that in SEQ ID NO:272. Optionally the anti-VSTM5 antibody or antibody fragment binds the same epitope as an anti-VSTM5 antibody comprising a variable heavy (VH) region identical to that in SEQ ID NO:271 and a variable light (VL) region identical to that in SEQ ID NO:272 and/or which elicits the same immunomodulatory effects as an anti-VSTM5 antibody comprising a variable heavy (VH) region identical to that in SEQ ID NO:271 and a variable light (VL) region identical to that in SEQ ID NO:272.

According to at least some embodiments, there is provided an anti-VSTM5 antibody or antibody fragment that comprises a variable heavy (VH) region at least 90, 95, 96, 97, 98, or 99% identical to that in SEQ ID NO:271 and/or a variable light (VL) region at least 90, 95, 96, 97, 98, or 99% identical to that in SEQ ID NO:272.

According to at least some embodiments, there is provided an anti-VSTM5 antibody or antibody fragment that comprises a variable heavy (VH) region identical to that in SEQ ID NO: 271 and/or a variable light (VL) region identical to that in SEQ ID NO: 272.

According to at least some embodiments, there is provided an anti-VSTM5 antibody or antibody fragment that comprises a VH region containing 1, 2 or 3 of the CDRs of SEQ ID NO:271 and/or a VL region containing 1, 2 or 3 of the CDRs of SEQ ID NO:272.

According to at least some embodiments, there is provided an anti-VSTM5 antibody or antibody fragment that comprises a VH region containing CDR 1, 2 and 3 polypeptides having the sequences of SEQ ID NO:331, 332 and 333, or a sequence at least 90, 95, 96, 97, 98, or 99% identical thereto, and a VL region containing CDR 1, 2 and 3 polypeptides having the sequences of SEQ ID NO. 334, 335 and 336, or a sequence at least 90, 95, 96, 97, 98, or 99% identical thereto.

According to at least some embodiments, there is provided an anti-VSTM5 antibody or antibody fragment that competes for binding with an anti-VSTM5 a variable heavy (VH) region identical to that in SEQ ID NO:273 and a variable light (VL) region identical to that in SEQ ID NO:274 to human VSTM5 or a human VSTM5 fragment or to a non-human VSTM5 ortholog and/or which elicits the same immunomodulatory effects as an anti-VSTM5 antibody comprising a variable heavy (VH) region identical to that in SEQ ID NO:273 and a variable light (VL) region identical to that in SEQ ID NO:274. Optionally the anti-VSTM5 antibody or antibody fragment binds the same epitope as an anti-VSTM5 antibody comprising a variable heavy (VH) region identical to that in SEQ ID NO:273 and a variable light (VL) region identical to that in SEQ ID NO:274 and/or which elicits the same immunomodulatory effects as an anti-VSTM5 antibody comprising a variable heavy (VH) region identical to that in SEQ ID NO:273 and a variable light (VL) region identical to that in SEQ ID NO:274.

According to at least some embodiments, there is provided an anti-VSTM5 antibody or antibody fragment that comprises a variable heavy (VH) region at least 90, 95, 96, 97, 98, or 99% identical to that in SEQ ID NO:273 and/or a variable light (VL) region at least 90, 95, 96, 97, 98, or 99% identical to that in SEQ ID NO:274.

According to at least some embodiments, there is provided an anti-VSTM5 antibody or antibody fragment that comprises a variable heavy (VH) region identical to that in SEQ ID NO: 273 and/or a variable light (VL) region identical to that in SEQ ID NO: 274.

According to at least some embodiments, there is provided an anti-VSTM5 antibody or antibody fragment that comprises a VH region containing 1, 2 or 3 of the CDRs of SEQ ID NO:273 and/or a VL region containing 1, 2 or 3 of the CDRs of SEQ ID NO:274.

According to at least some embodiments, there is provided an anti-VSTM5 antibody or antibody fragment that comprises a VH region containing CDR 1, 2 and 3 polypeptides having the sequences of SEQ ID NO:337, 338 and 339, or a sequence at least 90, 95, 96, 97, 98, or 99% identical thereto, and a VL region containing CDR 1, 2 and 3 polypeptides having the sequences of SEQ ID NO. 340, 341 and 342, or a sequence at least 90, 95, 96, 97, 98, or 99% identical thereto.

According to at least some embodiments, there is provided an anti-VSTM5 antibody or antibody fragment that competes for binding with an anti-VSTM5 a variable heavy (VH) region identical to that in SEQ ID NO:275 and a variable light (VL) region identical to that in SEQ ID NO:276 to human VSTM5 or a human VSTM5 fragment or to a non-human VSTM5 ortholog and/or which elicits the same immunomodulatory effects as an anti-VSTM5 antibody comprising a variable heavy (VH) region identical to that in SEQ ID NO:275 and a variable light (VL) region identical to that in SEQ ID NO:276. Optionally the anti-VSTM5 antibody or antibody fragment binds the same epitope as an anti-VSTM5 antibody comprising a variable heavy (VH) region identical to that in SEQ ID NO:275 and a variable light (VL) region identical to that in SEQ ID NO:276 and/or which elicits the same immunomodulatory effects as an anti-VSTM5 antibody comprising a variable heavy (VH) region identical to that in SEQ ID NO:275 and a variable light (VL) region identical to that in SEQ ID NO:276.

According to at least some embodiments, there is provided an anti-VSTM5 antibody or antibody fragment that comprises a variable heavy (VH) region at least 90, 95, 96, 97, 98, or 99% identical to that in SEQ ID NO:275 and/or a variable light (VL) region at least 90, 95, 96, 97, 98, or 99% identical to that in SEQ ID NO:276.

According to at least some embodiments, there is provided an anti-VSTM5 antibody or antibody fragment that comprises a variable heavy (VH) region identical to that in SEQ ID NO: 275 and/or a variable light (VL) region identical to that in SEQ ID NO: 276.

According to at least some embodiments, there is provided an anti-VSTM5 antibody or antibody fragment that comprises a VH region containing 1, 2 or 3 of the CDRs of SEQ ID NO:275 and/or a VL region containing 1, 2 or 3 of the CDRs of SEQ ID NO:276.

According to at least some embodiments, there is provided an anti-VSTM5 antibody or antibody fragment that comprises a VH region containing CDR 1, 2 and 3 polypeptides having the sequences of SEQ ID NO:343, 344 and 345, or a sequence at least 90, 95, 96, 97, 98, or 99% identical thereto, and a VL region containing CDR 1, 2 and 3 polypeptides having the sequences of SEQ ID NO. 346, 347 and 348, or a sequence at least 90, 95, 96, 97, 98, or 99% identical thereto.

According to at least some embodiments, there is provided an anti-VSTM5 antibody or antibody fragment that comprises a VH region containing CDR 1, 2 and 3 polypeptides, wherein said polypeptides are as follows: a heavy chain-CDR1 selected from the group consisting of: SEQ ID NOs: 181, 187, 193, 199, 205, 211, 217, 223, 229, 235, 241, 247, 277, 283, 289, 295, 301, 307, 313, 319, 325, 331, 337, and 343 or a polypeptide at least 90, 95, 96, 97, 98, or 99% identical thereto; a heavy chain-CDR2 selected from the group consisting of: SEQ ID NOs: 182, 188, 194, 200, 206, 212, 218, 224, 230, 236, 242, 248, 278, 284, 290, 296, 302, 308, 314, 320, 326, 332, 338, and 344 or a polypeptide at least 90, 95, 96, 97, 98, or 99% identical thereto; and a heavy chain-CDR3 selected from the group consisting of: SEQ ID NOs: 183, 189, 195, 201, 207, 213, 219, 225, 231, 237, 243, 249, 279, 285, 291, 297, 303, 309, 315, 321, 327, 333, 339, and 345 or a polypeptide at least 90, 95, 96, 97, 98, or 99% identical thereto.

According to at least some embodiments, there is provided an anti-VSTM5 antibody or antibody fragment that a VL region containing CDR 1, 2 and 3 polypeptides, wherein said polypeptides are as follows: light chain-CDR1 selected from the group consisting of: SEQ ID NOs: 184, 190, 196, 202, 208, 214, 220, 226, 232, 238, 244, 250, 280, 286, 292, 298, 304, 310, 316, 322, 328, 334, 340, and 346 or a polypeptide at least 90, 95, 96, 97, 98, or 99% identical thereto; a light chain-CDR2 selected from the group consisting of: SEQ ID NOs: 185, 191, 197, 203, 209, 215, 221, 227, 233, 239, 245, 251, 281, 287, 293, 299, 305, 311, 317, 323, 329, 335, 341, and 347 or a polypeptide at least 90, 95, 96, 97, 98, or 99% identical thereto; and a light chain-CDR3 selected from the group consisting of: SEQ ID NOs: 186, 192, 198, 204, 210, 216, 222, 228, 234, 240, 245, 252, 282, 288, 294, 300, 306, 312, 318, 324, 330, 336, 342, and 348 or a polypeptide at least 90, 95, 96, 97, 98, or 99% identical thereto.

According to at least some embodiments, there is provided an anti-VSTM5 antibody or antibody fragment that comprises a VH region containing CDR 1, 2 and 3 polypeptides and a VL region containing CDR 1, 2 and 3 polypeptides, wherein said polypeptides are selected according to any of the foregoing or as described herein.

According to at least some embodiments, there is provided an anti-VSTM5 antibody or antibody fragment that is derived by affinity maturation, chimerization, humanization, primatization, fusion or cleavage of an antibody according to any of the above claims. Optionally the anti-VSTM5 antibody or antigen-binding fragment thereof is derived by an affinity maturation procedure that includes systematically varying one or more residues in the VH or VL CDR1, 2 or 3 polypeptides. Optionally the anti-VSTM5 antibody or antigen-binding fragment thereof is derived by systematically varying one or more residues in the VH or VL CDR3 polypeptides.

According to at least some embodiments, there is provided an anti-VSTM5 antibody or antibody fragment that contains the same VH CDR3 as an antibody according to any of the foregoing or as described herein.

According to at least some embodiments, there is provided an anti-VSTM5 antibody or antibody fragment that contains the same VH CDR3 and VL CDR3 polypeptides as an antibody according to any of the foregoing or as described herein.

According to at least some embodiments there is provided an anti-VSTM5 antibody or antibody fragment that contains the same VH CDR2 and CDR3 and VL CDR2 and CDR3 polypeptides as an antibody according to any of the foregoing or as described herein.

According to at least some embodiments there is provided an anti anti-VSTM5 antibody or antigen-binding fragment according to any of the foregoing or as described herein wherein said antibody or antigen binding fragment is an immunomodulatory antibody or an immunomodulatory antigen-binding fragment thereof according to any of the foregoing or as described herein.

According to at least some embodiments there is provided an anti antibody or an antigen-binding fragment according to any of the foregoing or as described herein, which is selected from a chimeric, human, primatized, bispecific or humanized antibody.

According to at least some embodiments there is provided an anti antibody or an antigen-binding fragment according to any of the foregoing or as described herein, which comprises a human constant region.

Optionally said human constant region is a human IgG1, IgG2, IgG3 or IgG4 constant region or variant thereof, which optionally contains one or more domains deleted.

According to at least some embodiments there is provided an anti antibody or an antigen-binding fragment thereof according to any of the foregoing or as described herein, which comprises a human constant region which contains at least one mutation that increases or decreases an Fc effector function and/or glycosylation and/or a mutation which modulates or abrogates IgG4 Fab arm exchange.

Optionally said effector functions include FcR binding, ADCC activity, CDC activity, degranulation, phagocytosis, and cytokine release.

Optionally the anti-VSTM5 antibody or an antigen-binding fragment thereof according to any of the foregoing or as described herein is selected from the group consisting of a Fab, Fab′, F(ab′)2, F(ab′), F(ab), Fv or scFv fragment and a minimal recognition unit which optionally has an in vivo half-life of at least one week, 2 weeks, 3 weeks or a month.

According to at least some embodiments there is provided a humanized antibody or antibody fragment of an anti-VSTM5 antibody or an antigen-binding fragment thereof according to any of the foregoing or as described herein which optionally has an in vivo half-life of at least 1 week, 2 weeks, 3 weeks or a month.

According to at least some embodiments there is provided a human antibody or antibody fragment of an anti-VSTM5 antibody or an antigen-binding fragment thereof according to any of the foregoing or as described herein which optionally has an in vivo half-life of at least 1 week, 2 weeks, 3 weeks or a month.

According to at least some embodiments there is provided a bispecific antibody or antibody fragment of an anti-VSTM5 antibody or an antigen-binding fragment thereof according to any of the foregoing or as described herein, wherein one binding portion of the antibody is specific to a VSTM5 epitope and the other binding portion of the antibody is specific to another VSTM5 epitope or another antigen which optionally has an in vivo half-life of at least 1 week, 2 weeks, 3 weeks or a month.

According to at least some embodiments there is provided a primatized antibody or antibody fragment of an anti-VSTM5 antibody or an antigen-binding fragment thereof according to any of the foregoing or as described herein, which optionally has an in vivo half-life of at least one week, 2 weeks, 3 weeks or a month.

According to at least some embodiments there is provided a chimeric antibody or antibody fragment of an anti-VSTM5 antibody or an antigen-binding fragment thereof according to any of the foregoing or as described herein, which optionally has an in vivo half-life of at least 1 week, 2 weeks, 3 weeks or a month.

According to at least some embodiments there is provided an anti-VSTM5 antibody or an antigen-binding fragment thereof according to any of the foregoing or as described herein, which is coupled to another moiety.

According to at least some embodiments there is provided an anti-VSTM5 antibody or an antigen-binding fragment thereof according to any of the foregoing or as described herein, which is coupled to a therapeutic moiety, detectable moiety, or a moiety that alters (increases or decreases) in vivo half-life.

According to at least some embodiments there is provided an anti-VSTM5 antibody or an antigen-binding fragment thereof according to any of the foregoing or as described herein, which is coupled to a therapeutic agent selected from a drug, a radionuclide, a fluorophore, an enzyme, a toxin, or a chemotherapeutic agent; and/or a detectable marker selected from a radioisotope, a metal chelator, an enzyme, a fluorescent compound, a bioluminescent compound or a chemiluminescent compound.

According to at least some embodiments there is provided an anti-VSTM5 antibody or an antigen-binding fragment thereof according to any of the foregoing or as described herein, which is not coupled to any other moiety.

According to at least some embodiments there is provided an anti-VSTM5 antibody or an antigen-binding fragment thereof according to any of the foregoing or as described herein, which is not coupled to any other polypeptide moiety.

Optionally the antibody or antigen-binding fragment is coupled to an antibody or antigen-binding fragment thereof or other moiety which specifically binds to an NK and/or T cell receptor. Optionally the antibody or antigen-binding fragment thereof or other moiety which is coupled thereto specifically binds to an NK cell receptor that agonizes NK cell activity. Optionally the antibody or antigen-binding fragment thereof or other moiety which is coupled thereto specifically binds to an NK cell receptor that antagonizes NK cell activity.

Optionally the NK cell receptor is one that inhibits NK cell mediated cell depletion.

Optionally the inhibitory NK cell receptor is selected from the group consisting of KIR2DL1, KIR2DL2/3, KIR2DL4, KIR2DL5A, KIR2DL5B, KIR3DL1, KIR3DL2, KIR3DL3, LILRB1, NKG2A, NKG2C, NKG2E and LILRBS.

Optionally the NK cell receptor is one that promotes or activates NK cell mediated cell depletion.

Optionally the NK activating receptor is selected from the group consisting of NKp30, NKp44, NKp46, NKp46, NKG2D, KIR2DS4 CD2, CD16, CD69, DNAX accessory molecule-1 (DNAM-1), 2B4, NK1.1; a killer immunoglobulin (Ig)-like activating receptors (KAR); ILTs/LIRs; NKRP-1, CD69; CD94/NKG2C and CD94/NKG2E heterodimers, NKG2D homodimer KIR2DS and KIR3DS.

According to at least some embodiments there is provided an anti-VSTM5 antibody or an antigen-binding fragment according to any of the foregoing or as described herein which binds human or murine VSTM5 with a binding affinity (KD) no more than 500 nM as determined by any of the binding affinity methods disclosed herein.

According to at least some embodiments there is provided an anti-VSTM5 antibody or an antigen-binding fragment according to any of the foregoing or as described herein which binds human or murine VSTM5 with a binding affinity (KD) of about 10-5, 10-6, 10-7, 10-8, 10-9, 10-10, 10-11, 10-12M or less as determined by any of the binding affinity methods disclosed herein.

According to at least some embodiments there is provided an anti-VSTM5 antibody or an antigen-binding fragment according to any of the foregoing or as described herein, which binds human or murine VSTM5 with a binding affinity (KD) no more than 50 nM as determined by any of the binding affinity methods disclosed herein.

According to at least some embodiments there is provided an anti-VSTM5 antibody or an antigen-binding fragment according to any of the foregoing or as described herein wherein such antibody or antigen-binding fragment either (1) enhances, agonizes or mimics, or (2) inhibits, antagonizes or blocks at least one effect that a VSTM5 polypeptide having the amino acid sequence of SEQ ID NO: 2, 3, 6, 7, 132, or 349 elicits on immunity or on one or more types of immune cells.

Optionally the antibody or antigen-binding fragment inhibits, antagonizes or blocks at least one effect of a polypeptide (VSTM5) having the amino acid sequence of SEQ ID NO: 2, 3, 6, 7, 132, or 349 on immunity or on one or more types of immune cells.

Optionally the anti-VSTM5 antibody or the antigen-binding fragment mediates any combination of at least one of the following immunostimulatory effects on immunity: (i) increases immune response, (ii) increases T cell activation, (iii) increases cytotoxic T cell activity, (iv) increases NK cell activity, (v) alleviates T-cell suppression, (vi) increases pro-inflammatory cytokine secretion, (vii) increases IL-2 secretion; (viii) increases interferon-γ production, (ix) increases Th1 response, (x) decrease Th2 response, (xi) decreases or eliminates cell number and/or activity of at least one of regulatory T cells (Tregs), myeloid derived suppressor cells (MDSCs), iMCs, mesenchymal stromal cells, TIE2-expressing monocytes, (xii) reduces regulatory cell activity, and/or the activity of one or more of myeloid derived suppressor cells (MDSCs), iMCs, mesenchymal stromal cells, TIE2-expressing monocytes, (xiii) decreases or eliminates M2 macrophages, (xiv) reduces M2 macrophage pro-tumorigenic activity, (xv) decreases or eliminates N2 neutrophils, (xvi) reduces N2 neutrophils pro-tumorigenic activity, (xvii) reduces inhibition of T cell activation, (xviii) reduces inhibition of CTL activation, (xix) reduces inhibition of NK cell activation, (xx) reverses T cell exhaustion, (xxi) increases T cell response, (xxii) increases activity of cytotoxic cells, (xxiii) stimulates antigen-specific memory responses, (xxiv) elicits apoptosis or lysis of cancer cells, (xxv) stimulates cytotoxic or cytostatic effect on cancer cells, (xxvi) induces direct killing of cancer cells, (xxvii) increases Th17 activity and/or (xxviii) induces complement dependent cytotoxicity and/or antibody dependent cell-mediated cytotoxicity, with the proviso that said anti-VSTM5 antibody or antigen-binding fragment may elicit an opposite effect to one or more of (i)-(xxviii).

Optionally the immunomodulatory antibody or an antigen-binding fragment thereof inhibits, antagonizes or blocks at least one effect of VSTM5 on T or natural killer (NK) cell immunity.

Optionally the immunomodulatory antibody or an antigen-binding fragment thereof, suppresses the inhibitory effect of VSTM5 on T cell immunity.

Optionally the immunomodulatory antibody or an immunomodulatory antigen-binding fragment thereof promotes CTL activity.

Optionally CTL activity includes the secretion of one or more proinflammatory cytokines and/or CTL mediated killing of target cells.

Optionally the immunomodulatory antibody or an immunomodulatory antigen-binding fragment thereof promotes CD4⁺ T cell activation and/or CD4⁺ T cell proliferation and/or CD4⁺ T cell mediated cell depletion.

Optionally the immunomodulatory antibody or an immunomodulatory antigen-binding fragment thereof promotes CD8⁺ T cell activation and/or CD8⁺ T cell proliferation and/or CD8⁺ T cell mediated cell depletion.

Optionally the immunomodulatory antibody or an immunomodulatory antigen-binding fragment thereof enhances NK cell activity, and/or NK cell proliferation and/or NK cell mediated cell depletion.

Optionally enhanced NK cell activity includes increased depletion of target cells and/or proinflammatory cytokine release.

Optionally the immunomodulatory antibody or an immunomodulatory antigen-binding fragment thereof decreases or eliminates the differentiation, proliferation and/or activity of regulatory cells (Tregs), and/or the differentiation, proliferation, infiltration and/or activity of myeloid derived suppressor cells (MDSCs).

Optionally the immunomodulatory antibody or an immunomodulatory antigen-binding fragment thereof decreases or eliminates the infiltration of inducible Tregs (iTregs) into a target site.

Optionally said target site is a cancer cell, tissue or organ, tumor draining lymph node, or an infectious disease site or lesion.

Optionally the immunomodulatory antibody or an immunomodulatory antigen-binding fragment thereof promotes NK mediated cell depletion.

Optionally the immunomodulatory antibody or an immunomodulatory antigen-binding fragment promotes anti-tumor immunity by suppressing one or more of the effects of VSTM5 on immunity.

Optionally the immunomodulatory antibody or an immunomodulatory antigen-binding fragment promotes an immune response against an infectious agent by suppressing one or more of the effects of VSTM5 on immunity.

Optionally the anti-VSTM5 antibody or the antigen-binding fragment, or the immunomodulatory antibody or the immunomodulatory antigen-binding fragment, is provided for use in treatment of cancer.

Optionally the anti-VSTM5 antibody or the antigen-binding fragment, or the immunomodulatory antibody or the immunomodulatory antigen-binding fragment, is provided for use in treatment of infectious disease.

Optionally the antibody or antigen-binding fragment enhances, agonizes or mimics at least one effect of a polypeptide (VSTM5) having the amino acid sequence of SEQ ID NO: 2, 3, 6, 7, 132, or 349 on immunity or immune cells.

Optionally the anti-VSTM5 antibody or the antigen-binding fragment mediates any combination of at least one of the following immunoinhibitory effects: (i) decreases immune response, (ii) decreases T cell activation, (iii) decreases cytotoxic T cell activity, (iv) decreases natural killer (NK) cell activity, (v) decreases T-cell activity, (vi) decreases pro-inflammatory cytokine secretion, (vii) decreases IL-2 secretion; (viii) decreases interferon-γ production, (ix) decreases Th1 response, (x) decreases Th2 response, (xi) increases cell number and/or activity of regulatory T cells, (xii) increases regulatory cell activity and/or one or more of myeloid derived suppressor cells (MDSCs), iMCs, mesenchymal stromal cells, TIE2-expressing monocytes, (xiii) increases regulatory cell activity and/or the activity of one or more of myeloid derived suppressor cells (MDSCs), iMCs, mesenchymal stromal cells, TIE2-expressing monocytes, (xiii) increases M2 macrophages, (xiv) increases M2 macrophage activity, (xv) increases N2 neutrophils, (xvi) increases N2 neutrophils activity, (xvii) increases inhibition of T cell activation, (xviii) increases inhibition of CTL activation, (xix) increases inhibition of NK cell activation, (xx) increases T cell exhaustion, (xxi) decreases T cell response, (xxii) decreases activity of cytotoxic cells, (xxiii) reduces antigen-specific memory responses, (xxiv) inhibits apoptosis or lysis of cells, (xxv) decreases cytotoxic or cytostatic effect on cells, (xxvi) reduces direct killing of cells, (xxvii) decreases Th17 activity, and/or (xxviii) reduces complement dependent cytotoxicity and/or antibody dependent cell-mediated cytotoxicity, with the proviso that said anti-VSTM5 antibody or the antigen-binding fragment may elicit an opposite effect to one or more of (i)-(xxviii).

Optionally the immunomodulatory antibody or an antigen-binding fragment thereof enhances, agonizes or mimics at least one effect of VSTM5 on T or natural killer (NK) cell immunity.

Optionally the immunomodulatory antibody or an antigen-binding fragment thereof increases the inhibitory effect of VSTM5 on T cell immunity.

Optionally the immunomodulatory antibody or an antigen-binding fragment thereof inhibits CTL activity.

Optionally inhibited CTL activity includes reduced secretion of one or more proinflammatory cytokines and/or reduced CTL mediated killing of target cells.

Optionally the immunomodulatory antibody or an immunomodulatory antigen-binding fragment thereof inhibits CD4⁺ T cell activation and/or CD4⁺ T cell proliferation and/or CD4⁺ T cell mediated cell depletion.

Optionally the immunomodulatory antibody or an immunomodulatory antigen-binding fragment thereof inhibits CD8⁺ T cell activation and/or CD8⁺ T cell proliferation and/or CD8⁺ T cell mediated cell depletion.

Optionally the immunomodulatory antibody or an immunomodulatory antigen-binding fragment thereof, inhibits NK cell activity, and/or NK cell proliferation and/or NK cell mediated cell depletion.

Optionally inhibited NK cell activity includes reduced depletion of target cells and/or proinflammatory cytokine release.

Optionally the immunomodulatory antibody or an immunomodulatory antigen-binding fragment thereof increases the differentiation, proliferation and/or activity of regulatory T cells (Tregs) and/or the differentiation, proliferation, infiltration and/or activity of myeloid derived suppressor cells (MDSC's).

Optionally the immunomodulatory antibody or an immunomodulatory antigen-binding fragment thereof increases the infiltration of Tregs or MDSCs into a disease site.

Optionally the disease site is a transplanted cell, tissue or organ, or an autoimmune, allergic, or inflammatory site or lesion.

Optionally the immunomodulatory antibody or an immunomodulatory antigen-binding fragment thereof inhibits an allergic, autoimmune or inflammatory immune response by promoting one or more of the effects of VSTM5 on immunity.

Optionally the immunomodulatory antibody or an immunomodulatory antigen-binding fragment thereof promotes antigen-specific tolerance or prolonged suppression of an antigen-specific immune response by enhancing one or more of the effects of VSTM5 on immunity.

Optionally the immunomodulatory antibody or an immunomodulatory antigen-binding fragment thereof elicits tolerance or prolonged suppression of antigen-specific immunity against transplanted cells, tissue or organ.

Optionally the immunomodulatory antibody or an immunomodulatory antigen-binding fragment thereof inhibits an immune response against an autoantigen, allergen, or inflammatory agent by promoting one or more of the effects of VSTM5 on immunity.

Optionally the anti-VSTM5 antibody or the antigen-binding fragment, or the immunomodulatory antibody or the immunomodulatory antigen-binding fragment, is provided for use in inhibiting an immune response against an autoantigen, allergen, or inflammatory agent, and/or for treating an inflammatory disease or response and/or for treating an autoimmune disease and/or for reducing or prevent transplant rejection and/or graft vs host disease.

According to at least some embodiments, there is provided a pharmaceutical composition comprising at least one antibody or antigen-binding fragment thereof according to any of the foregoing or as described herein.

According to at least some embodiments, there is provided a vaccine composition comprising at least one antibody or antigen-binding fragment thereof according to any of the foregoing or as described herein and an antigen.

Optionally said at least one antibody or antigen-binding fragment thereof is immunomodulatory.

According to at least some embodiments, there is provided an immunosuppressive vaccine composition comprising at least one antibody or antigen-binding fragment thereof according to any of the foregoing or as described herein, wherein said antibody or antigen-binding fragment thereof in said composition suppresses antigen-specific T and/or B cell immunity or induces tolerance.

Optionally the antigen to which immunity is suppressed is a human antigen, tumor antigen, infectious agent antigen, autoantigen, or an allergen.

Optionally the composition further comprises a human antigen, cell or antigen of a cell, tissue, or organ to be transplanted into a subject, autoantigen, inflammatory agent or an allergen.

Optionally said at least one antibody or antigen-binding fragment thereof is immunomodulatory.

Optionally the composition is suitable for administration by a route selected from oral, topical, or injection.

Optionally the composition is suitable for administration by a route selected from intravascular delivery (e.g. injection or infusion), intravenous, intramuscular, intradermal, intraperitoneal, subcutaneous, spinal, oral, enteral, rectal, pulmonary (e.g. inhalation), nasal, topical (including transdermal, buccal and sublingual), intravesical, intravitreal, intraperitoneal, vaginal, brain delivery (e.g. intra-cerebroventricular, intra-cerebral, and convection enhanced diffusion), CNS delivery (e.g. intrathecal, perispinal, and intra-spinal) or parenteral (including subcutaneous, intramuscular, intravenous and intradermal), transmucosal (e.g., sublingual administration), administration or administration via an implant, or other parenteral routes of administration, wherein “parenteral administration” refers to modes of administration other than enteral and topical administration.

Optionally the composition is suitable for administration by a route selected from, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Optionally the composition is suitable for intraperitoneal, subcutaneous or intravenous administration.

Optionally the composition comprises at least one other active agent, e.g., a therapeutic or diagnostic agent.

Optionally the other active agent is selected from another immunomodulatory compound, a chemotherapeutic, a drug, a cytokine, a radionuclide, and an enzyme.

Optionally the composition comprises an antigen that is expressed by a target cell (e.g., a tumor or infected cell).

Optionally the composition comprises or is used with another composition containing at least one immunomodulatory agent selected from PD-1 agonists and antagonists, PD-L1 and PD-L2 antibodies and antibody fragments, TLR agonists, CD40 agonists or antagonists, VISTA agonists or antagonists, CTLA-4 fusion proteins, CD28 agonists or antagonists, 4-1BB agonists or antagonists, CD27 or CD70 agonists or antagonists, LAGS agonists or antagonists, TIM3 agonists or antagonists, TIGIT agonists or antagonists, ICOS agonists or antagonists, ICOS ligand agonists or antagonists.

According to at least some embodiments, there is provided a method of treatment and/or diagnosis, or use of a composition containing an anti-VSTM5 antibody or antigen-binding fragment for diagnostic or therapeutic use, which method or use comprises the administration to a subject in need thereof at least one dosage or composition comprising a therapeutically or diagnostically effective amount of at least one anti-VSTM5 antibody, antigen-binding fragment or composition containing such according to any of the foregoing or as described herein.

According to at least some embodiments, there is provided a diagnostic method or use of an antibody or antigen-binding fragment or composition containing in detecting whether an individual has a condition associated with an increase or decrease in VSTM5-mediated effects on immunity wherein the method or use includes contacting a tissue sample from the individual with an antibody, or antigen-binding fragment or composition according to any of the foregoing or as described herein, and detecting specific binding thereto.

Optionally the disease is selected from the group consisting of cancer, autoimmune disease, or infectious disease,

Optionally the method or use detects the upregulation of VSTM5 expression and/or increased number of VSTM5 expressing cells.

Optionally the method or use detects the downregulation of VSTM5 expression and/or the decreased number of VSTM5 expressing cells.

According to at least some embodiments, there is provided a diagnostic method or use of an anti-VSTM5 antibody or antigen-binding fragment or composition containing which includes detecting whether an individual has a condition associated with an increase or decrease in VSTM5-mediated effects on immunity comprising contacting a tissue sample from the individual with an antibody, or antigen-binding fragment or composition according to any of the foregoing or as described herein wherein the diagnostic method is performed in vivo, comprising administering to the subject with an immunomodulatory antibody, or antigen-binding fragment or composition according to any of the foregoing or as described herein and detecting specific binding thereto.

Optionally the disease is selected from the group consisting of cancer, autoimmune disease, inflammatory condition, allergic condition or an infectious disease.

According to at least some embodiments, there is provided a diagnostic method or use which includes an anti-VSTM5 antibody or antigen-binding fragment or composition containing, and which method or use includes diagnosing a disease in a subject, wherein the disease is selected from the group consisting of cancer, autoimmune disease, or an infectious disease wherein the diagnostic method is performed ex vivo or in vivo, comprising contacting a sample from the individual or administering the individual an antibody, or antigen-binding fragment or composition according to any of the foregoing or as described herein, and detecting specific binding of the immune molecule or antibody of any of the above claims to a tissue of the subject.

Optionally the diagnostic method or use is performed before administering to the individual a therapeutically effective amount of an antibody, antigen-binding fragment, or immunomodulatory polypeptide or pharmaceutical composition containing such according to any of the foregoing or as described herein.

Optionally a therapeutically effective amount of an antibody, antigen-binding fragment, or immunomodulatory polypeptide or a pharmaceutical composition containing according to any of the foregoing or as described herein is only administered if the individual has a condition characterized by increased expression of VSTM5 by diseased and/or APC cells and/or increased numbers of diseased and/or APC cells which express VSTM5.

Optionally the expression level of VSTM5 is detected by conducting an IHC (immunohistochemistry) assay or a gene expression assay with a tissue of the subject.

Optionally said IHC assay comprises determining if a level of expression is at least 1 on a scale of 0 to 3.

Optionally VSTM5 expression is detected on one or more of cancer cells, immune infiltrate or stromal cells.

Optionally VSTM5 expression levels are determined by contacting tissues of the individual with an antibody or antigen-binding fragment or composition according to any of the foregoing or as described herein and detecting specific binding thereto.

According to at least some embodiments, there is provided a diagnostic method or use of an anti-VSTM5 antibody or antigen-binding fragment, which method or use includes diagnosing whether a tissue sample taken from a subject exhibits an immune condition associated with increased or decreased VSTM5 expression, comprising (i) contacting the sample with an antibody or antibody fragment or composition according to any of the foregoing or as described herein, or with a nucleic acid that detects VSTM5 expression and (ii) conducting a binding or amplification assay that detects VSTM5 expression, and (iii) based thereon diagnosing whether the sample is from an individual with a condition associated with an immune condition associated with increased or decreased VSTM5 expression.

Optionally the immune condition is selected from the group consisting of cancer, autoimmune disease, inflammatory condition, an allergic condition, an infectious disease or sepsis.

Optionally the method or use is used for screening for a disease, detecting a presence or a severity of a disease, providing prognosis of a disease, aiding in the diagnosis of a disease, monitoring disease progression or relapse, as well as assessment of treatment efficacy and/or relapse of a disease, disorder or condition, as well as selecting a therapy and/or a treatment for a disease, optimization of a given therapy for a disease, monitoring the treatment of a disease, and/or predicting the suitability of a therapy for specific patients or subpopulations or determining the appropriate dosing of a therapeutic product in patients or subpopulations.

Optionally the method or use detects the expression of at least one other marker wherein the expression thereof correlates to the particular disease that is being screened.

Optionally said anti-VSTM5 antibody or antigen-binding fragment is an immunostimulatory antibody which mediates any combination of at least one of the following immunostimulatory effects on immunity: (i) increases immune response, (ii) increases T cell activation, (iii) increases cytotoxic T cell activity, (iv) increases NK cell activity, (v) alleviates T-cell suppression, (vi) increases pro-inflammatory cytokine secretion, (vii) increases IL-2 secretion; (viii) increases interferon-γ production, (ix) increases Th1 response, (x) decrease Th2 response, (xi) decreases or eliminates cell number and/or activity of at least one of regulatory T cells (Tregs), myeloid derived suppressor cells (MDSCs), iMCs, mesenchymal stromal cells, TIE2-expressing monocytes, (xii) reduces regulatory cell activity, and/or the activity of one or more of myeloid derived suppressor cells (MDSCs), iMCs, mesenchymal stromal cells, TIE2-expressing monocytes, (xiii) decreases or eliminates M2 macrophages, (xiv) reduces M2 macrophage pro-tumorigenic activity, (xv) decreases or eliminates N2 neutrophils, (xvi) reduces N2 neutrophils pro-tumorigenic activity, (xvii) reduces inhibition of T cell activation, (xviii) reduces inhibition of CTL activation, (xix) reduces inhibition of NK cell activation, (xx) reverses T cell exhaustion, (xxi) increases T cell response, (xxii) increases activity of cytotoxic cells, (xxiii) stimulates antigen-specific memory responses, (xxiv) elicits apoptosis or lysis of cancer cells, (xxv) stimulates cytotoxic or cytostatic effect on cancer cells, (xxvi) induces direct killing of cancer cells, (xxvii) increases Th17 activity and/or (xxviii) induces complement dependent cytotoxicity and/or antibody dependent cell-mediated cytotoxicity, with the proviso that said anti-VSTM5 antibody or antigen-binding fragment may elicit an opposite effect to one or more of (i)-(xxviii).

According to at least some embodiments, there is provided a method of treatment and/or diagnosis, or use of a composition containing an anti-VSTM5 antibody or antigen-binding fragment for diagnostic or therapeutic use, which comprises promoting T cell immunity or natural killer (NK) immunity and/or suppressing Tregs or MDSC's in a subject in need thereof, which comprises administering a therapeutically or diagnostically effective amount of at least one antibody, antigen-binding fragment or a composition containing according to any of the foregoing or as described herein, wherein such antibody or antigen-binding fragment inhibits, antagonizes or blocks at least one effect of a polypeptide (VSTM5) having the amino acid sequence of SEQ ID NO: 2, 3, 6, 7, 132, or 349 on immunity or immune cells.

Optionally the method or use suppresses the inhibitory effect of VSTM5 on T cell immunity.

Optionally the method or use promotes CTL activity.

Optionally the method or use CTL activity includes the secretion of one or more proinflammatory cytokines and/or CTL mediated killing of target cells.

Optionally the method or use promotes CD4⁺ T cell activation and/or CD4⁺ T cell proliferation and/or CD4⁺ T cell mediated cell depletion.

Optionally the method or use promotes CD8⁺ T cell activation and/or CD8⁺ T cell proliferation and/or CD8⁺ T cell mediated cell depletion.

Optionally the method or use enhances NK cell activity. Optionally enhanced NK cell activity includes increased depletion of target cells and/or proinflammatory cytokine release.

Optionally the method or use suppresses and or decreases the differentiation, proliferation and/or activity of regulatory cells, such as Tregs and/or the differentiation, proliferation, infiltration and/or activity myeloid derived suppressor cells (MDSCs).

Optionally the method or use suppresses and/or decreases the infiltration of infiltration of regulatory cells, such as Tregs and MDSCs into a target site.

Optionally said target site is a transplanted cell, tissue or organ, or an autoimmune, allergic or inflammatory site or lesion.

Optionally the method or use promotes NK mediated cell depletion.

Optionally the method or use promotes anti-tumor immunity by suppressing one or more of the effects of VSTM5 on immunity.

Optionally the method or use is used in the treatment of cancer, sepsis or an infectious condition or combination thereof.

According to at least some embodiments, the method of treatment and/or diagnosis and/or diagnosis, or use of a composition containing an anti-VSTM5 antibody or antigen-binding fragment for diagnostic or therapeutic use, which comprises promoting NK or T cell immunity in a subject in need thereof, and which comprises administering a therapeutically or diagnostically effective amount of at least one antibody, antigen-binding fragment or a composition containing according to any of the foregoing or as described herein, wherein such antibody or antigen-binding fragment inhibits at least one effect of a polypeptide (VSTM5) having the amino acid sequence of SEQ ID NO: 2, 3, 6, 7, 132, 349, or a polypeptide having at least 90% sequence identity therewith or to a non-human VSTM5 ortholog on immunity or immune cells.

Optionally the treated individual suffers from an infectious disease.

Optionally the infectious disease is caused by a virus, bacterium, parasite, nematode, yeast, mycoplasm, fungus or prion.

Optionally the infectious disease is caused by a Retroviridae (e.g., human immunodeficiency viruses, such as HIV-1 or HIV-2, acquired immune deficiency (AIDS) also referred to as HTLV-III, LAV or HTLV-III/LAV, or HIV-III; and other isolates, such as HIV-LP; Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever virus); Reoviridae (e.g., reoviruses, orbiviruses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herperviridae (herpes simplex virus (HSV) 1 and 2, varicella zoster virus, cytomegalovirus (CMV), herpes viruses); Poxviridae (variola virsues, vaccinia viruses, pox viruses); and Iridoviridae (e.g., African swine fever virus); an unclassified virus (e.g., the etiological agents of Spongiform encephalopathies, the agent of delta hepatitides, the agents of non-A, non-B hepatitis (class 1—internally transmitted; class 2—parenterally transmitted (i.e., Hepatitis C); Norwalk and related viruses, and astroviruses) as well as Severe acute respiratory syndrome virus and respiratory syncytial virus (RSV), West Nile encephalitis, coronavirus infection, rhinovirus infection, influenza, dengue, hemorrhagic fever; an otological infection; severe acute respiratory syndrome (SARS), acute febrile pharyngitis, pharyngoconjunctival fever, epidemic keratoconjunctivitis, infantile gastroenteritis, infectious mononucleosis, Burkitt lymphoma, acute hepatitis, chronic hepatitis, hepatic cirrhosis, hepatocellular carcinoma, primary HSV-1 infection, (gingivostomatitis in children, tonsillitis & pharyngitis in adults, keratoconjunctivitis), latent HSV-1 infection (herpes labialis, cold sores), aseptic meningitis, Cytomegalovirus infection, Cytomegalic inclusion disease, Kaposi sarcoma, Castleman disease, primary effusion lymphoma, influenza, measles, encephalitis, postinfectious encephalomyelitis, Mumps, hyperplastic epithelial lesions (common, flat, plantar and anogenital warts, laryngeal papillomas, epidermodysplasia verruciformis), croup, pneumonia, bronchiolitis, Poliomyelitis, Rabies, bronchiolitis, pneumonia, German measles, congenital rubella, Hemorrhagic Fever, Chickenpox, Dengue, Ebola infection, Echovirus infection, EBV infection, Fifth Disease, Filovirus, Flavivirus, Hand, foot & mouth disease, Herpes Zoster Virus (Shingles), Human Papilloma Virus Associated Epidermal Lesions, Lassa Fever, Lymphocytic choriomeningitis, Parainfluenza Virus Infection, Paramyxovirus, Parvovirus B19 Infection, Picornavirus, Poxviruses infection, Rotavirus diarrhea, Rubella, Rubeola, Varicella, Variola infection.

Optionally the infectious disease is a parasite infection caused by a parasite selected from a protozoa, such as Amebae, Flagellates, Plasmodium falciparum, Toxoplasma gondii, Ciliates, Coccidia, Microsporidia, Sporozoa; helminthes, Nematodes (Roundworms), Cestodes (Tapeworms), Trematodes (Flukes), Arthropods, and aberrant proteins known as prions.

Optionally the infectious disease is an infectious disorder and/or disease caused by bacteria selected from the group consisting of Sepsis, septic shock, sinusitis, skin infections, pneumonia, bronchitis, meningitis, Bacterial vaginosis, Urinary tract infection (UCI), Bacterial gastroenteritis, Impetigo and erysipelas, Erysipelas, Cellulitis, anthrax, whooping cough, lyme disease, Brucellosis, enteritis, acute enteritis, Tetanus, diphtheria, Pseudomembranous colitis, Gas gangrene, Acute food poisoning, Anaerobic cellulitis, Nosocomial infections, Diarrhea, Meningitis in infants, Traveller's diarrhea, Hemorrhagic colitis, Hemolytic-uremic syndrome, Tularemia, Peptic ulcer, Gastric and Duodenal ulcers, Legionnaire's Disease, Pontiac fever, Leptospirosis, Listeriosis, Leprosy (Hansen's disease), Tuberculosis, Gonorrhea, Ophthalmia neonatorum, Septic arthritis, Meningococcal disease including meningitis, Waterhouse-Friderichsen syndrome, Pseudomonas infection, Rocky mountain spotted fever, Typhoid fever type salmonellosis, Salmonellosis with gastroenteritis and enterocolitis, Bacillary dysentery/Shigellosis, Coagulase-positive staphylococcal infections: Localized skin infections including Diffuse skin infection (Impetigo), Deep localized infections, Acute infective endocarditis, Septicemia, Necrotizing pneumonia, Toxinoses such as Toxic shock syndrome and Staphylococcal food poisoning, Cystitis, Endometritis, Otitis media, Streptococcal pharyngitis, Scarlet fever, Rheumatic fever, Puerperal fever, Necrotizing fasciitis, Cholera, Plague (including Bubonic plague and Pneumonic plague), as well as any infection caused by a bacteria selected from but not limited to Helicobacter pyloris, Boreliai burgdorferi, Legionella pneumophila, Mycobacteria sps (e.g., M. tuberculosis, M. avium, M. intracellulare, M. kansaii, M gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidum, Treponema pertenue, Leptospira, and Actinomyces israelii.

Optionally the infectious disease is an infectious disorder and/or disease caused by fungi selected from Allergic bronchopulmonary aspergillosis, Aspergilloma, Aspergillosis, Basidiobolomycosis, Blastomycosis, Candidiasis, Chronic pulmonary aspergillosis, Chytridiomycosis, Coccidioidomycosis, Conidiobolomycosis, Covered smut (barley), Cryptococcosis, Dermatophyte, Dermatophytid, Dermatophytosis, Endothrix, Entomopathogenic fungus, Epizootic lymphangitis, Epizootic ulcerative syndrome, Esophageal candidiasis, Exothrix, Fungemia, Histoplasmosis, Lobomycosis, Massospora cicadina, Mycosis, Mycosphaerella fragariae, Myringomycosis, Paracoccidioidomycosis, Pathogenic fungi, Penicilliosis, Thousand cankers disease, Tinea, Zeaspora, Zygomycosis; a parasite selected from the group consisting of but not limited to Acanthamoeba, Amoebiasis, Ascariasis, Ancylostomiasis, Anisakiasis, Babesiosis, Balantidiasis, Baylisascariasis, Blastocystosis, Candiru, Chagas disease, Clonorchiasis, Cochliomyia, Coccidia, Chinese Liver Fluke Cryptosporidiosis, Dientamoebiasis, Diphyllobothriasis, Dioctophyme renalis infection, Dracunculiasis, Echinococcosis, Elephantiasis, Enterobiasis, Fascioliasis, Fasciolopsiasis, Filariasis, Giardiasis, Gnathostomiasis, Hymenolepiasis, Halzoun Syndrome, Isosporiasis, Katayama fever, Leishmaniasis, lymphatic filariasis, Malaria, Metagonimiasis, Myiasis, Onchocerciasis, Pediculosis, Primary amoebic meningoencephalitis, Parasitic pneumonia, Paragonimiasis, Scabies, Schistosomiasis, Sleeping sickness, Strongyloidiasis, Sparganosis, Rhinosporidiosis, River blindness, Taeniasis (cause of Cysticercosis), Toxocariasis, Toxoplasmosis, Trichinosis, Trichomoniasis, Trichuriasis, Trypanosomiasis, Tapeworm infection, Cryptococcus neoformans, Histoplasma capsulatum, Coccidioides immitis, Blastomyces dermatitidis, Chlamydia trachomatis, Candida albicans.

Optionally the infectious disease is caused by any of hepatitis B, hepatitis C, infectious mononucleosis, EBV, cytomegalovirus, AIDS, HIV-1, HIV-2, tuberculosis, malaria and schistosomiasis.

According to at least some embodiments, there is provided anti-VSTM5 antibody or antigen-binding fragment or composition, or method or use according to any of the foregoing or as described herein, which includes another therapeutic agent useful for treating bacterial infection, viral infection, fungal infection, parasitic infection or sepsis.

Optionally the method, composition, antibody or fragment, or use promotes an immune response against an infectious agent by suppressing one or more of the effects of VSTM5 on immunity.

Optionally the method, composition, antibody or fragment, or use further comprises one or more additional therapeutic agents used for treatment of bacterial infections.

Optionally said agent is selected from the group consisting of antibiotics including Aminoglycosides, Carbapenems, Cephalosporins, Macrolides, Lincosamides, Nitrofurans, penicillins, Polypeptides, Quinolones, Sulfonamides, Tetracyclines, drugs against mycobacteria including but not limited to Clofazimine, Cycloserine, Cycloserine, Rifabutin, Rifapentine, Streptomycin and other antibacterial drugs such as Chloramphenicol, Fosfomycin, Metronidazole, Mupirocin, and Tinidazole, or a combination thereof.

Optionally the method, composition, antibody or fragment, or use further comprises one or more additional therapeutic agents used for treatment of viral infections.

Optionally said agent is selected from the group consisting of antiviral drugs such as oseltamivir (brand name Tamiflu®) and zanamivir (brand name Relenza®) Arbidol®—adamantane derivatives (Amantadine®, Rimantadine®)—neuraminidase inhibitors (Oseltamivir®, Laninamivir®, Peramivir®, Zanamivir®) nucleotide analog reverse transcriptase inhibitor including Purine analogue guanine (Aciclovir®/Valacyclovir®, Ganciclovir®/Valganciclovir®, Penciclovir®/Famciclovir®) and adenine (Vidarabine®), Pyrimidine analogue, uridine (Idoxuridine®, Trifluridine®, Edoxudine®), thymine (Brivudine®), cytosine (Cytarabine®); Foscarnet; Nucleoside analogues/NARTIs: Entecavir, Lamivudine®, Telbivudine®, Clevudine®; Nucleotide analogues/NtRTIs: Adefovir®, Tenofovir; Nucleic acid inhibitors such as Cidofovir®; InterferonInterferon alfa-2b, Peginterferon α-2a; Ribavirin®/Taribavirin®; antiretroviral drugs including zidovudine, lamivudine, abacavir, lopinavir, ritonavir, tenofovir/emtricitabine, efavirenz each of them alone or a various combinations, gp41 (Enfuvirtide), Raltegravir®, protease inhibitors such as Fosamprenavir®, Lopinavir® and Atazanavir®, Methisazone®, Docosanol®, Fomivirsen®, and Tromantadine®.

Optionally the method, composition, antibody or fragment, or use further comprises one or more additional therapeutic agents used for treatment of fungal infections.

Optionally the agent is selected from the group consisting of antifungal drugs of the Polyene antifungals, Imidazole, triazole, and thiazole antifungals, Allylamines, Echinocandins or other anti-fungal drugs.

Optionally the treated individual suffers from cancer.

Optionally the cancer is selected from the group consisting of breast cancer, cervical cancer, ovary cancer, endometrial cancer, melanoma, uveal melanoma, bladder cancer, lung cancer, pancreatic cancer, colorectal cancer, prostate cancer, leukemia, acute lymphocytic leukemia, chronic lymphocytic leukemia, B-cell lymphoma, Burkitt's lymphoma, multiple myeloma, Non-Hodgkin's lymphoma, myeloid leukemia, acute myelogenous leukemia (AML), chronic myelogenous leukemia, thyroid cancer, thyroid follicular cancer, myelodysplastic syndrome (MDS), fibrosarcomas and rhabdomyosarcomas, teratocarcinoma, neuroblastoma, glioma, glioblastoma, benign tumor of the skin, keratoacanthomas, renal cancer, anaplastic large-cell lymphoma, esophageal cancer, follicular dendritic cell carcinoma, seminal vesicle tumor, epidermal carcinoma, spleen cancer, bladder cancer, head and neck cancer, stomach cancer, liver cancer, bone cancer, brain cancer, cancer of the retina, biliary cancer, small bowel cancer, salivary gland cancer, cancer of uterus, cancer of testicles, cancer of connective tissue, myelodysplasia, Waldenström's macroglobinaemia, nasopharyngeal, neuroendocrine cancer, mesothelioma, angiosarcoma, Kaposi's sarcoma, carcinoid, fallopian tube cancer, peritoneal cancer, papillary serous müllerian cancer, malignant ascites, gastrointestinal stromal tumor (GIST), Li-Fraumeni syndrome and Von Hippel-Lindau syndrome (VHL), cancer of unknown origin either primary or metastatic, wherein the cancer is non-metastatic, invasive or metastatic.

Optionally the cancer is selected from B-cell lymphoma, Burkitt's lymphoma, thyroid cancer, thyroid follicular cancer, myelodysplastic syndrome (MDS), fibrosarcomas and rhabdomyosarcomas, melanoma, uveal melanoma, teratocarcinoma, neuroblastoma, glioma, glioblastoma cancer, keratoacanthomas, anaplastic large-cell lymphoma, esophageal squamous cells carcinoma, hepatocellular carcinoma cancer, follicular dendritic cell carcinoma, muscle-invasive cancer, seminal vesicle tumor, epidermal carcinoma, cancer of the retina, biliary cancer, small bowel cancer, salivary gland cancer, cancer of connective tissue, myelodysplasia, Waldenström's macroglobinaemia, nasopharyngeal, neuroendocrine cancer, myelodysplastic syndrome, mesothelioma, angiosarcoma, Kaposi's sarcoma, carcinoid, esophagogastric, fallopian tube cancer, peritoneal cancer, papillary serous müllerian cancer, malignant ascites, gastrointestinal stromal tumor (GIST), Li-Fraumeni syndrome and Von Hippel-Lindau syndrome (VHL); endometrial cancer, Breast carcinoma, preferably any of ductal-carcinoma, infiltrating ductal carcinoma, lobular carcinoma, mucinous adenocarcinoma, intra duct and invasive ductal carcinoma, and Scirrhous adenocarcinoma, Colorectal adenocarcinoma, preferably any of Poorly to Well Differentiated invasive and noninvasive Adenocarcinoma, Poorly to Well Differentiated Adenocarcinoma of the cecum, Well to Poorly Differentiated Adenocarcinoma of the colon, Tubular adenocarcinoma, preferably Grade 2 Tubular adenocarcinoma of the ascending colon, colon adenocarcinoma Duke's stage C1, invasive adenocarcinoma, Adenocarcinoma of the rectum, preferably Grade 3 Adenocarcinoma of the rectum, Moderately Differentiated Adenocarcinoma of the rectum, Moderately Differentiated Mucinous adenocarcinoma of the rectum; Lung cancer, preferably any of Well to Poorly differentiated Non-small cell carcinoma, Squamous Cell Carcinoma, preferably well to poorly Differentiated Squamous Cell Carcinoma, keratinizing squamous cell carcinoma, adenocarcinoma, preferably poorly to well differentiated adenocarcinoma, large cell adenocarcinoma, Small cell lung cancer, preferably Small cell lung carcinoma, more preferably undifferentiated Small cell lung carcinoma; Prostate adenocarcinoma, preferably any of Adenocarcinoma Gleason Grade 6 to 9, Infiltrating adenocarcinoma, High grade prostatic intraepithelial neoplasia, undifferentiated carcinoma; Stomach adenocarcinoma, preferably moderately differentiated gastric adenocarcinoma; Ovary carcinoma, preferably any of cystadenocarcinoma, serous papillary cystic carcinoma, Serous papillary cystic carcinoma, Invasive serous papillary carcinoma; Brain cancer, preferably any of Astrocytoma, with the proviso that it is not a grade 2 astrocytoma, preferably grade 4 Astrocytoma, Glioblastoma multiforme; Kidney carcinoma, preferably Clear cell renal cell carcinoma; Liver cancer, preferably any of Hepatocellular carcinoma, preferably Low Grade hepatocellular carcinoma, Fibrolamellar Hepatocellular Carcinoma; Lymphoma, preferably any of, Hodgkin's Lymphoma and High to low grade Non-Hodgkin's Lymphoma and with the proviso that if the cancer is brain cancer, it is not Astrocytoma grade 2, and if the cancer is Non-Hodgkin's Lymphoma, it is not a large cell Non-Hodgkin's Lymphoma, and wherein the cancer is non-metastatic, invasive or metastatic.

Optionally said breast cancer is breast carcinoma, and is selected from the group consisting of ductal-carcinoma, infiltrating ductal carcinoma, lobular carcinoma, mucinous adenocarcinoma, intra duct and invasive ductal carcinoma, and Scirrhous adenocarcinoma.

Optionally the cancer is a colon cancer selected from the group consisting of Poorly to Well Differentiated invasive and non-invasive Adenocarcinoma, Poorly to Well Differentiated Adenocarcinoma of the cecum, Well to Poorly Differentiated Adenocarcinoma of the colon, Tubular adenocarcinoma, preferably Grade 2 Tubular adenocarcinoma of the ascending colon, colon adenocarcinoma Duke's stage C1, invasive adenocarcinoma, Adenocarcinoma of the rectum, preferably Grade 3 Adenocarcinoma of the rectum, Moderately Differentiated Adenocarcinoma of the rectum, Moderately Differentiated Mucinous adenocarcinoma of the rectum.

Optionally the cancer is a cancer is selected from the group consisting of Well to Poorly differentiated Non-small cell carcinoma, Squamous Cell Carcinoma, preferably well to poorly Differentiated Squamous Cell Carcinoma, keratinizing squamous cell carcinoma, adenocarcinoma, preferably poorly to well differentiated adenocarcinoma, large cell adenocarcinoma, Small cell lung cancer, preferably Small cell lung carcinoma, more preferably undifferentiated Small cell lung carcinoma.

Optionally the cancer is a prostate adenocarcinoma selected from the group consisting of Adenocarcinoma Gleason Grade 6 to 9, Infiltrating adenocarcinoma, High grade prostatic intraepithelial neoplasia, undifferentiated carcinoma.

Optionally the cancer is a stomach cancer comprising moderately differentiated gastric adenocarcinoma.

Optionally the cancer is an ovarian cancer selected from the group consisting of cystadenocarcinoma, serous papillary cystic carcinoma, Serous papillary cystic carcinoma, Invasive serous papillary carcinoma.

Optionally the cancer is a brain cancer selected from the group consisting Astrocytoma, with the proviso that it is not a grade 2 astrocytoma, preferably grade 4 Astrocytoma, and Glioblastoma multiforme.

Optionally the cancer is clear cell renal cell carcinoma.

Optionally the cancer is Hepatocellular carcinoma.

Optionally the cancer is a Hepatocellular carcinoma selected from Low Grade hepatocellular carcinoma and Fibrolamellar Hepatocellular Carcinoma.

Optionally the cancer is a lymphoma selected from the group consisting of Hodgkin's Lymphoma and High to low grade Non-Hodgkin's Lymphoma.

Optionally the levels of VSTM5 protein are elevated compared to normal cell samples.

Optionally the treated individual suffers from a cancer wherein the cancer or other cells contained at the tumor sites do not express VSTM5 protein or do not express VSTM5 protein at levels higher than normal.

Optionally the treated subject suffers from a cancer wherein the diseased cells, APC's or other cells at the disease site express VSTM5 protein.

According to at least some embodiments, the anti-VSTM5 antibody or antigen-binding fragment or composition, or method or use according to any of the foregoing or as disclosed, which includes treatment with an anti-VSTM5 antibody or antigen-binding fragment or composition containing and the therapy comprises one or more of radiotherapy, cryotherapy, antibody therapy, chemotherapy, photodynamic therapy, surgery, hormonal deprivation or combination therapy with conventional drugs.

According to at least some embodiments, the anti-VSTM5 antibody or antigen-binding fragment or composition, or method or use according to any of the foregoing or as disclosed which includes treatment with an anti-VSTM5 antibody or antigen-binding fragment or composition containing and another therapeutic agent selected from the group consisting of cytotoxic drugs, tumor vaccines, antibodies, peptides, pepti-bodies, small molecules, chemotherapeutic agents, cytotoxic and cytostatic agents, immunological modifiers, interferons, interleukins, immunostimulatory growth hormones, cytokines, vitamins, minerals, aromatase inhibitors, RNAi, Histone Deacetylase Inhibitors, and proteasome inhibitors.

According to at least some embodiments, the anti-VSTM5 antibody or antigen-binding fragment or composition, or method or use according to any of the foregoing or as disclosed which includes treatment with an anti-VSTM5 antibody or antigen-binding fragment or composition containing and another therapeutic or an imaging agent administered to a subject simultaneously or sequentially in combination with one or more potentiating agents to obtain a therapeutic effect, wherein said one or more potentiating agents is selected from the group consisting of radiotherapy, conventional/classical anti-cancer therapy potentiating anti-tumor immune responses, Targeted therapy potentiating anti-tumor immune responses, Therapeutic agents targeting immunosuppressive cells Tregs and/or MDSCs, Immunostimulatory antibodies, Cytokine therapy, Adoptive cell transfer.

Optionally the conventional/classical anti-cancer agent is selected from platinum based compounds, antibiotics with anti-cancer activity, Anthracyclines, Anthracenediones, alkylating agents, antimetabolites, Antimitotic agents, Taxanes, Taxoids, microtubule inhibitors, Vinca alkaloids, Folate antagonists, Topoisomerase inhibitors, Antiestrogens, Antiandrogens, Aromatase inhibitors, GnRh analogs, inhibitors of 5α-reductase, biphosphonates.

Optionally the anti-VSTM5 antibody or antigen-binding fragment or composition, or method or use further comprises Platinum based compounds such as oxaliplatin, cisplatin, carboplatin; Antibiotics with anti-cancer activity, such as dactinomycin, bleomycin, mitomycin-C, mithramycin and Anthracyclines, such as doxorubicin, daunorubicin, epirubicin, idarubicin; Anthracenediones, such as mitoxantrone; Alkylating agents, such as dacarbazine, melphalan, cyclophosphamide, temozolomide, chlorambucil, busulphan, nitrogen mustard, nitrosoureas; Antimetabolites, such as fluorouracil, raltitrexed, gemcitabine, cytosine arabinoside, hydroxyurea and Folate antagonists, such as methotrexate, trimethoprim, pyrimethamine, pemetrexed; Antimitotic agents such as polokinase inhibitors and Microtubule inhibitors, such as Taxanes and Taxoids, such as paclitaxel, docetaxel; Vinca alkaloids such as vincristine, vinblastine, vindesine, vinorelbine; Topoisomerase inhibitors, such as etoposide, teniposide, amsacrine, topotecan, irinotecan, camptothecin; Cytostatic agents including Antiestrogens such as tamoxifen, fulvestrant, toremifene, raloxifene, droloxifene, iodoxyfene, Antiandrogens such as bicalutamide, flutamide, nilutamide and cyproterone acetate, Progestogens such as megestrol acetate, Aromatase inhibitors such as anastrozole, letrozole, vorozole, exemestane; GnRH analogs, such as leuprorelin, goserelin, buserelin, degarelix; inhibitors of 5α-reductase such as finasteride.

Optionally the anti-VSTM5 antibody or antigen-binding fragment or composition, or method or use further comprises a targeted therapy selected from the group consisting of but not limited to: histone deacetylase (HDAC) inhibitors, such as vorinostat, romidepsin, panobinostat, belinostat, mocetinostat, abexinostat, entinostat, resminostat, givinostat, quisinostat, sodium butyrate; Proteasome inhibitors, such as bortezomib, carfilzomib, disulfiram; mTOR pathway inhibitors, such as temsirolimus, rapamycin, everolimus; PI3K inhibitors, such as perifosine, CAL101, PX-866, IPI-145, BAY 80-6946; B-raf inhibitors such as vemurafenib, sorafenib; JAK2 inhibitors, such as lestaurtinib, pacritinib; Tyrosine kinase inhibitors (TKIs), such as erlotinib, imatinib, sunitinib, lapatinib, gefitinib, sorafenib, nilotinib, toceranib, bosutinib, neratinib, vatalanib, regorafenib, cabozantinib; other Protein kinase inhibitors, such as crizotinib; Inhibitors of serine/threonine kinases for example Ras/Raf signalling inhibitors such as farnesyl transferase inhibitors; Inhibitors of serine proteases for example matriptase, hepsin, urokinase; Inhibitors of intracellular signaling such as tipifarnib, perifosine; Inhibitors of cell signalling through MEK and/or AKT kinases; aurora kinase inhibitors such as AZD1152, PH739358, VX-680, MLN8054, R763, MP235, MP529, VX-528, AX39459; Cyclin dependent kinase inhibitors such as CDK2 and/or CDK4 inhibitors; Inhibitors of survival signaling proteins including Bcl-2, Bcl-XL, such as ABT-737; HSP90 inhibitors; Therapeutic monoclonal antibodies, such as anti-EGFR mAbs cetuximab, panitumumab, nimotuzumab, anti-ERBB2 mAbs trastuzumab, pertuzumab, anti-CD20 mAbs such as rituximab, ofatumumab, veltuzumab and mAbs targeting other tumor antigens such as alemtuzumab, labetuzumab, adecatumumab, oregovomab, onartuzumab; TRAIL pathway agonists, such as dulanermin (soluble rhTRAIL), apomab, mapatumumab, lexatumumab, conatumumab, tigatuzumab; Antibody fragments, bi-specific antibodies and bi-specific T-cell engagers (BiTEs), such as catumaxomab, blinatumomab; Antibody drug conjugates (ADC) and other immunoconjugates, such as ibritumomab triuxetan, tositumomab, brentuximab vedotin, gemtuzumab ozogamicin, clivatuzumab tetraxetan, pemtumomab, trastuzumab emtansine; Anti-angiogenic therapy such as bevacizumab, etaracizumab, volociximab, ramucirumab, aflibercept, sorafenib, sunitinib, regorafenib, axitinib, nintedanib, motesanib, pazopanib, cediranib; Metalloproteinase inhibitors such as marimastat; Inhibitors of urokinase plasminogen activator receptor function; Inhibitors of cathepsin activity.

238) Optionally the another therapeutic agent is another antibody selected from cetuximab, panitumumab, nimotuzumab, trastuzumab, pertuzumab, rituximab, ofatumumab, veltuzumab, alemtuzumab, labetuzumab, adecatumumab, oregovomab, onartuzumab; apomab, mapatumumab, lexatumumab, conatumumab, tigatuzumab, catumaxomab, blinatumomab, ibritumomab triuxetan, tositumomab, brentuximab vedotin, gemtuzumab ozogamicin, clivatuzumab tetraxetan, pemtumomab, trastuzumab emtansine, bevacizumab, etaracizumab, volociximab, ramucirumab, aflibercept.

Optionally the anti-VSTM5 antibody or antigen-binding fragment or composition, or method or use further comprises a Therapeutic cancer vaccine selected from exogenous cancer vaccines including proteins or peptides used to mount an immunogenic response to a tumor antigen, recombinant virus and bacteria vectors encoding tumor antigens, DNA-based vaccines encoding tumor antigens, proteins targeted to dendritic cell-based vaccines, whole tumor cell vaccines, gene modified tumor cells expressing GM-CSF, ICOS and/or Flt3-ligand, oncolytic virus vaccines.

Optionally the anti-VSTM5 antibody or antigen-binding fragment or composition, or method or use further comprises a Cytokine therapy selected from one or more of the following cytokines such as IL-2, IL-7, IL-12, IL-15, IL-17, IL-18 and IL-21, IL-23, IL-27, GM-CSF, IFNα (interferon α), IFNα-2b, IFNβ, IFNγ, and their different strategies for delivery.

Optionally the anti-VSTM5 antibody or antigen-binding fragment or composition, or method or use further comprises adoptive cell transfer therapy which is carried out following ex vivo treatment selected from expansion of the patient autologous naturally occurring tumor specific T cells or genetic modification of T cells to confer specificity for tumor antigens.

Optionally said anti-VSTM5 antibody or antigen-binding fragment comprises an immunoinhibitory antibody or an antigen-binding fragment which mediates any combination of at least one of the following immunoinhibitory effects: (i) decreases immune response, (ii) decreases T cell activation, (iii) decreases cytotoxic T cell activity, (iv) decreases natural killer (NK) cell activity, (v) decreases T-cell activity, (vi) decreases pro-inflammatory cytokine secretion, (vii) decreases IL-2 secretion; (viii) decreases interferon-γ production, (ix) decreases Th1 response, (x) decreases Th2 response, (xi) increases cell number and/or activity of regulatory T cells, (xii) increases regulatory cell activity and/or one or more of myeloid derived suppressor cells (MDSCs), iMCs, mesenchymal stromal cells, TIE2-expressing monocytes, (xiii) increases regulatory cell activity and/or the activity of one or more of myeloid derived suppressor cells (MDSCs), iMCs, mesenchymal stromal cells, TIE2-expressing monocytes, (xiii) increases M2 macrophages, (xiv) increases M2 macrophage activity, (xv) increases N2 neutrophils, (xvi) increases N2 neutrophils activity, (xvii) increases inhibition of T cell activation, (xviii) increases inhibition of CTL activation, (xix) increases inhibition of NK cell activation, (xx) increases T cell exhaustion, (xxi) decreases T cell response, (xxii) decreases activity of cytotoxic cells, (xxiii) reduces antigen-specific memory responses, (xxiv) inhibits apoptosis or lysis of cells, (xxv) decreases cytotoxic or cytostatic effect on cells, (xxvi) reduces direct killing of cells, (xxvii) decreases Th17 activity, and/or (xxviii) reduces complement dependent cytotoxicity and/or antibody dependent cell-mediated cytotoxicity, with the proviso that said anti-VSTM5 antibody or antigen-binding fragment may elicit an opposite effect to one or more of (i)-(xxviii).

According to at least some embodiments, there is provided a method of treatment and/or diagnosis, or use of a composition containing an anti-VSTM5 antibody or antigen-binding fragment for diagnostic or therapeutic use, which comprises suppressing T cell immunity or natural killer (NK) immunity and/or promoting Tregs or MDSC's in a subject in need thereof, which comprises administering a therapeutically or diagnostically effective amount of at least one antibody, antigen-binding fragment or a composition containing according to any of the foregoing or as described herein, wherein such antibody or antigen-binding fragment agonizes, mimics or promotes at least one effect of a polypeptide (VSTM5) having the amino acid sequence of SEQ ID NO: 2, 3, 6, 7, 132, or 349 on immunity or immune cells.

Optionally the method or use is used in the treatment of allergy, autoimmunity, transplant, gene therapy, inflammation or combination thereof.

Optionally the treated individual has or is to receive cell therapy, gene therapy or a transplanted tissue or organ, and the treatment reduces or inhibits the undesirable immune activation that is associated with such cell therapy, gene.

Optionally the antibody, or antigen-binding fragment thereof is an immunoinhibitory antibody or fragment which effects one or more of the following: (i) decreases immune response, (ii) decreases T cell activation, (iii) decreases cytotoxic T cell activity, (iv) decreases natural killer (NK) cell activity, (v) decreases T-cell activity, (vi) decreases pro-inflammatory cytokine secretion, (vii) decreases IL-2 secretion; (viii) decreases interferon-γ production, (ix) decreases Th1 response, (x) decreases Th2 response, (xi) increases cell number and/or activity of regulatory T cells, (xii) increases regulatory cell activity and/or one or more of myeloid derived suppressor cells (MDSCs), iMCs, mesenchymal stromal cells, TIE2-expressing monocytes, (xiii) increases regulatory cell activity and/or the activity of one or more of myeloid derived suppressor cells (MDSCs), iMCs, mesenchymal stromal cells, TIE2-expressing monocytes, (xiii) increases M2 macrophages, (xiv) increases M2 macrophage activity, (xv) increases N2 neutrophils, (xvi) increases N2 neutrophils activity, (xvii) increases inhibition of T cell activation, (xviii) increases inhibition of CTL activation, (xix) increases inhibition of NK cell activation, (xx) increases T cell exhaustion, (xxi) decreases T cell response, (xxii) decreases activity of cytotoxic cells, (xxiii) reduces antigen-specific memory responses, (xxiv) inhibits apoptosis or lysis of cells, (xxv) decreases cytotoxic or cytostatic effect on cells, (xxvi) reduces direct killing of cells, (xxvii) decreases Th17 activity, and/or (xxviii) reduces complement dependent cytotoxicity and/or antibody dependent cell-mediated cytotoxicity, with the proviso that said anti-VSTM5 antibody or antigen-binding fragment may elicit an opposite effect to one or more of (i)-(xxviii).

Optionally, the method or use enhances, agonizes or mimics at least one effect of VSTM5 on T or natural killer (NK) cell immunity.

Optionally, the method or use increases the inhibitory effect of VSTM5 on T cell immunity.

Optionally, the method or use inhibits CTL activity.

Optionally inhibited CTL activity includes reduced secretion of one or more proinflammatory cytokines and/or reduced CTL mediated killing of target cells.

Optionally, the method or use inhibits CD4⁺ T cell activation and/or CD4⁺ T cell proliferation and/or CD4⁺ T cell mediated cell depletion.

Optionally, the method or use inhibits CD8⁺ T cell activation and/or CD8⁺ T cell proliferation and/or CD8⁺ T cell mediated cell depletion.

Optionally, the method or use inhibits NK cell activity.

Optionally inhibited NK cell activity includes reduced depletion of target cells and/or proinflammatory cytokine release.

Optionally, the method or use promotes and/or increases the differentiation, proliferation and/or activity of regulatory cells, such as T cells (Tregs) and/or the differentiation, proliferation, infiltration and/or activity of myeloid derived suppressor cells (MDSC's).

Optionally, the method or use promotes and/or increases the infiltration of regulatory cells, such as Tregs or MDSCs into a disease site.

Optionally, the method or use inhibits an allergic, autoimmune or inflammatory immune response by promoting one or more of the effects of VSTM5 on immunity.

Optionally, the method or use promotes antigen-specific tolerance or prolonged suppression of an antigen-specific immune response by enhancing one or more of the effects of VSTM5 on immunity.

Optionally, the method or use elicits tolerance or prolonged suppression of antigen-specific immunity against transplanted cells, tissue or organ.

Optionally, the method or use inhibits an immune response against an autoantigen, allergen, or inflammatory agent by promoting one or more of the effects of VSTM5 on immunity.

Optionally the treated individual has or is to receive cell therapy, gene therapy or a transplanted tissue or organ, and the treatment reduces or inhibits the undesirable immune activation that is associated with such cell therapy, gene therapy or a transplanted tissue or organ.

Optionally, the method or use is used to treat an inflammatory or autoimmune disorder or a condition associated with inflammation selected from Acid Reflux/Heartburn, Acne, Acne Vulgaris, Allergies and Sensitivities, Alzheimer's Disease, Asthma, Atherosclerosis and Vascular Occlusive Disease, optionally Atherosclerosis, Ischemic Heart Disease, Myocardial Infarction, Stroke, Peripheral Vascular Disease, or Vascular Stent Restenosis, Autoimmune Diseases, Bronchitis, Cancer, Carditis, Cataracts, Celiac Disease, Chronic Pain, Chronic Prostatitis, Cirrhosis, Colitis, Connective Tissue Diseases, optionally Systemic Lupus Erythematosus, Systemic Sclerosis, Polymyositis, Dermatomyositis, or Sjögren's Syndrome and related conditions such as Sjögren's syndrome” herein includes one or more of Sjögren's syndrome, Primary Sjögren's syndrome and Secondary Sjögren's syndrome, as well as conditions or complications relating to Sjögren's syndrome including connective tissue disease, such as rheumatoid arthritis, systemic lupus erythematosus, or scleroderma, pneumonia, pulmonary fibrosis, interstitial nephritis, inflammation of the tissue around the kidney's filters, glomerulonephritis, renal tubular acidosis, carpal tunnel syndrome, peripheral neuropathy, cranial neuropathy, primary biliary cirrhosis (PBC), cirrhosis, Inflammation in the esophagus, stomach, pancreas, and liver (including hepatitis), Polymyositis, Raynaud's phenomenon, Vasculitis, Autoimmune thyroid problems, lymphoma, Corneal Disease, Crohn's Disease, Crystal Arthropathies, optionally Gout, Pseudogout, Calcium Pyrophosphate Deposition Disease, Dementia, Dermatitis, Diabetes, Dry Eyes, Eczema, Edema, Emphysema, Fibromyalgia, Gastroenteritis, Gingivitis, Glomerulonephritis, Heart Disease, Hepatitis, High Blood Pressure, Hypersensitivities, Inflammatory Bowel Diseases, Inflammatory Conditions including Consequences of Trauma or Ischaemia, Insulin Resistance, Interstitial Cystitis, Iridocyclitis, Iritis, Joint Pain, Arthritis, Lyme Disease, Metabolic Syndrome (Syndrome X), Multiple Sclerosis, Myositis, Nephritis, Obesity, Ocular Diseases including Uveitis, Osteopenia, Osteoporosis, Parkinson's Disease, Pelvic Inflammatory Disease, Periodontal Disease, Polyarteritis, Polychondritis, Polymyalgia Rheumatica, Psoriasis, Reperfusion Injury, Rheumatic Arthritis, Rheumatic Diseases, Rheumatoid Arthritis, Osteoarthritis, or Psoriatic Arthritis, Rheumatoid Arthritis, Sarcoidosis, Scleroderma, Sinusitis, “Sjögren's syndrome” and related conditions or complications associated therewith such as one or more of Sjögren's syndrome, Primary Sjögren's syndrome and Secondary Sjögren's syndrome, conditions relating to Sjögren's syndrome including connective tissue disease, such as rheumatoid arthritis, systemic lupus erythematosus, or scleroderma, and complications relating to Sjögren's syndrome such as pneumonia, pulmonary fibrosis, interstitial nephritis, inflammation of the tissue around the kidney's filters, glomerulonephritis, renal tubular acidosis, carpal tunnel syndrome, peripheral neuropathy, cranial neuropathy, primary biliary cirrhosis (PBC), cirrhosis, inflammation in the esophagus, stomach, pancreas, and liver (including hepatitis), Polymyositis, Raynaud's phenomenon, Vasculitis, Autoimmune thyroid problems, lymphoma, Sjögren's Syndrome, Spastic Colon, Spondyloarthropathies, optionally Ankylosing Spondylitis, Reactive Arthritis, or Reiter's Syndrome, Systemic Candidiasis, Tendonitis, Transplant Rejection, UTI's, Vaginitis, Vascular Diseases including Atherosclerotic Vascular Disease, Vasculitides, Polyarteritis Nodosa, Wegener's Granulomatosis, Churg-Strauss Syndrome, or vasculitis.

Optionally, the method or use is used to treat an autoimmune or allergic disease selected from acute anterior uveitis, Acute Disseminated Encephalomyelitis (ADEM), acute gouty arthritis, acute necrotizing hemorrhagic leukoencephalitis, acute or chronic sinusitis, acute purulent meningitis (or other central nervous system inflammatory disorders), acute serious inflammation, Addison's disease, adrenalitis, adult onset diabetes mellitus (Type II diabetes), adult-onset idiopathic hypoparathyroidism (AOIH), Agammaglobulinemia, agranulocytosis, vasculitides, including vasculitis, optionally, large vessel vasculitis, optionally, polymyalgia rheumatica and giant cell (Takayasu's) arthritis, allergic conditions, allergic contact dermatitis, allergic dermatitis, allergic granulomatous angiitis, allergic hypersensitivity disorders, allergic neuritis, allergic reaction, alopecia greata, alopecia totalis, Alport's syndrome, alveolitis, optionally allergic alveolitis or fibrosing alveolitis, Alzheimer's disease, amyloidosis, amylotrophic lateral sclerosis (ALS; Lou Gehrig's disease), an eosinophil-related disorder, optionally eosinophilia, anaphylaxis, ankylosing spondylitis, angiectasis, antibody-mediated nephritis, Anti-GBM/Anti-TBM nephritis, antigen-antibody complex-mediated diseases, antiglomerular basement membrane disease, anti-phospholipid antibody syndrome, antiphospholipid syndrome (APS), aphthae, aphthous stomatitis, aplastic anemia, arrhythmia, arteriosclerosis, arteriosclerotic disorders, arthritis, optionally rheumatoid arthritis such as acute arthritis, or chronic rheumatoid arthritis, arthritis chronica progrediente, arthritis deformans, ascariasis, aspergilloma, granulomas containing eosinophils, aspergillosis, aspermiogenese, asthma, optionally asthma bronchiale, bronchial asthma, or auto-immune asthma, ataxia telangiectasia, ataxic sclerosis, atherosclerosis, autism, autoimmune angioedema, autoimmune aplastic anemia, autoimmune atrophic gastritis, autoimmune diabetes, autoimmune disease of the testis and ovary including autoimmune orchitis and oophoritis, autoimmune disorders associated with collagen disease, autoimmune dysautonomia, autoimmune ear disease, optionally autoimmune inner ear disease (AGED), autoimmune endocrine diseases including thyroiditis such as autoimmune thyroiditis, autoimmune enteropathy syndrome, autoimmune gonadal failure, autoimmune hearing loss, autoimmune hemolysis, Autoimmune hepatitis, autoimmune hepatological disorder, autoimmune hyperlipidemia, autoimmune immunodeficiency, autoimmune inner ear disease (AIED), autoimmune myocarditis, autoimmune neutropenia, autoimmune pancreatitis, autoimmune polyendocrinopathies, autoimmune polyglandular syndrome type I, autoimmune retinopathy, autoimmune thrombocytopenic purpura (ATP), autoimmune thyroid disease, autoimmune urticaria, autoimmune-mediated gastrointestinal diseases, Axonal & neuronal neuropathies, Balo disease, Behçet's disease, benign familial and ischemia-reperfusion injury, benign lymphocytic angiitis, Berger's disease (IgA nephropathy), bird-fancier's lung, blindness, Boeck's disease, bronchiolitis obliterans (non-transplant) vs NSIP, bronchitis, bronchopneumonic aspergillosis, Bruton's syndrome, bullous pemphigoid, Caplan's syndrome, Cardiomyopathy, cardiovascular ischemia, Castleman's syndrome, Celiac disease, celiac sprue (gluten enteropathy), cerebellar degeneration, cerebral ischemia, and disease accompanying vascularization, Chagas disease, channelopathies, optionally epilepsy, channelopathies of the CNS, chorioretinitis, choroiditis, an autoimmune hematological disorder, chronic active hepatitis or autoimmune chronic active hepatitis, chronic contact dermatitis, chronic eosinophilic pneumonia, chronic fatigue syndrome, chronic hepatitis, chronic hypersensitivity pneumonitis, chronic inflammatory arthritis, Chronic inflammatory demyelinating polyneuropathy (CIDP), chronic intractable inflammation, chronic mucocutaneous candidiasis, chronic neuropathy, optionally IgM polyneuropathies or IgM-mediated neuropathy, chronic obstructive airway disease, chronic pulmonary inflammatory disease, Chronic recurrent multifocal osteomyelitis (CRMO), chronic thyroiditis (Hashimoto's thyroiditis) or subacute thyroiditis, Churg-Strauss syndrome, cicatricial pemphigoid/benign mucosal pemphigoid, CNS inflammatory disorders, CNS vasculitis, Coeliac disease, Cogan's syndrome, cold agglutinin disease, colitis polyposa, colitis such as ulcerative colitis, colitis ulcerosa, collagenous colitis, conditions involving infiltration of T cells and chronic inflammatory responses, congenital heart block, congenital rubella infection, Coombs positive anemia, coronary artery disease, Coxsackie myocarditis, CREST syndrome (calcinosis, Raynaud's phenomenon), Crohn's disease, cryoglobulinemia, Cushing's syndrome, cyclitis, optionally chronic cyclitis, heterochronic cyclitis, iridocyclitis, or Fuch's cyclitis, cystic fibrosis, cytokine-induced toxicity, deafness, degenerative arthritis, demyelinating diseases, optionally autoimmune demyelinating diseases, demyelinating neuropathies, dengue, dermatitis herpetiformis and atopic dermatitis, dermatitis including contact dermatitis, dermatomyositis, dermatoses with acute inflammatory components, Devic's disease (neuromyelitis optica), diabetic large-artery disorder, diabetic nephropathy, diabetic retinopathy, Diamond Blackfan anemia, diffuse interstitial pulmonary fibrosis, dilated cardiomyopathy, discoid lupus, diseases involving leukocyte diapedesis, Dressler's syndrome, Dupuytren's contracture, echovirus infection, eczema including allergic or atopic eczema, encephalitis such as Rasmussen's encephalitis and limbic and/or brainstem encephalitis, encephalomyelitis, optionally allergic encephalomyelitis or encephalomyelitis allergica and experimental allergic encephalomyelitis (EAE), endarterial hyperplasia, endocarditis, endocrine ophthalmopathy, endometriosis. endomyocardial fibrosis, endophthalmia phacoanaphylactica, endophthalmitis, enteritis allergica, eosinophilia-myalgia syndrome, eosinophilic fascitis, epidemic keratoconjunctivitis, epidermolysis bullosa acquisita (EBA), episclera, episcleritis, Epstein-Barr virus infection, erythema elevatum et diutinum, erythema multiforme, erythema nodosum leprosum, erythema nodosum, erythroblastosis fetalis, esophageal dysmotility, Essential mixed cryoglobulinemia, ethmoid, Evan's syndrome, Experimental Allergic Encephalomyelitis (EAE), Factor VIII deficiency, farmer's lung, febris rheumatica, Felty's syndrome, fibromyalgia, fibrosing alveolitis, filariasis, focal segmental glomerulosclerosis (FSGS), food poisoning, frontal, gastric atrophy, giant cell arthritis (temporal arthritis), giant cell hepatitis, giant cell polymyalgia, glomerulonephritides, glomerulonephritis (GN) with and without nephrotic syndrome such as chronic or acute glomerulonephritis (e.g., primary GN), Goodpasture's syndrome, gouty arthritis, granulocyte transfusion-associated syndromes, granulomatosis including lymphomatoid granulomatosis, granulomatosis with polyangiitis (GPA), granulomatous uveitis, Grave's disease, Guillain-Barre syndrome, gutatte psoriasis, hemoglobinuria paroxysmatica, Hamman-Rich's disease, Hashimoto's disease, Hashimoto's encephalitis, Hashimoto's thyroiditis, hemochromatosis, hemolytic anemia or immune hemolytic anemia including autoimmune hemolytic anemia (AIHA), hemolytic anemia, hemophilia A, Henoch-Schönlein purpura, Herpes gestationis, human immunodeficiency virus (HIV) infection, hyperalgesia, hypogammaglobulinemia, hypogonadism, hypoparathyroidism, idiopathic diabetes insipidus, idiopathic facial paralysis, idiopathic hypothyroidism, idiopathic IgA nephropathy, idiopathic membranous GN or idiopathic membranous nephropathy, idiopathic nephritic syndrome, idiopathic pulmonary fibrosis, idiopathic sprue, Idiopathic thrombocytopenic purpura (ITP), IgA nephropathy, IgE-mediated diseases, optionally anaphylaxis and allergic or atopic rhinitis, IgG4-related sclerosing disease, ileitis regionalis, immune complex nephritis, immune responses associated with acute and delayed hypersensitivity mediated by cytokines and T-lymphocytes, immune-mediated GN, immunoregulatory lipoproteins, including adult or acute respiratory distress syndrome (ARDS), Inclusion body myositis, infectious arthritis, infertility due to antispermatozoan antibodies, inflammation of all or part of the uvea, inflammatory bowel disease (IBD) inflammatory hyperproliferative skin diseases, inflammatory myopathy, insulin-dependent diabetes (type1), insulitis, Interstitial cystitis, interstitial lung disease, interstitial lung fibrosis, iritis, ischemic re-perfusion disorder, joint inflammation, Juvenile arthritis, juvenile dermatomyositis, juvenile diabetes, juvenile onset (Type I) diabetes mellitus, including pediatric insulin-dependent diabetes mellitus (IDDM), juvenile-onset rheumatoid arthritis, Kawasaki syndrome, keratoconjunctivitis sicca, kypanosomiasis, Lambert-Eaton syndrome, leishmaniasis, leprosy, leucopenia, leukocyte adhesion deficiency, Leukocytoclastic vasculitis, leukopenia, lichen planus, lichen sclerosus, ligneous conjunctivitis, linear IgA dermatosis, Linear IgA disease (LAD), Loffler's syndrome, lupoid hepatitis, lupus (including nephritis, cerebritis, pediatric, non-renal, extra-renal, discoid, alopecia), Lupus (SLE), lupus erythematosus disseminatus, Lyme arthritis, Lyme disease, lymphoid interstitial pneumonitis, malaria, male and female autoimmune infertility, maxillary, medium vessel vasculitis (including Kawasaki's disease and polyarteritis nodosa), membrano- or membranous proliferative GN (MPGN), including Type I and Type II, and rapidly progressive GN, membranous GN (membranous nephropathy), Meniere's disease, meningitis, microscopic colitis, microscopic polyangiitis, migraine, minimal change nephropathy, Mixed connective tissue disease (MCTD), mononucleosis infectiosa, Mooren's ulcer, Mucha-Habermann disease, multifocal motor neuropathy, multiple endocrine failure, multiple organ injury syndrome such as those secondary to septicemia, trauma or hemorrhage, multiple organ injury syndrome, multiple sclerosis (MS) such as spino-optical MS, multiple sclerosis, mumps, muscular disorders, myasthenia gravis such as thymoma-associated myasthenia gravis, myasthenia gravis, myocarditis, myositis, narcolepsy, necrotizing enterocolitis, and transmural colitis, and autoimmune inflammatory bowel disease, necrotizing, cutaneous, or hypersensitivity vasculitis, neonatal lupus syndrome (NLE), nephrosis, nephrotic syndrome, neurological disease, neuromyelitis optica (Devic's), neuromyelitis optica, neuromyotonia, neutropenia, non-cancerous lymphocytosis, nongranulomatous uveitis, non-malignant thymoma, ocular and orbital inflammatory disorders, ocular cicatricial pemphigoid, oophoritis, ophthalmia symphatica, opsoclonus myoclonus syndrome (OMS), opsoclonus or opsoclonus myoclonus syndrome (OMS), and sensory neuropathy, optic neuritis, orchitis granulomatosa, osteoarthritis, palindromic rheumatism, pancreatitis, pancytopenia, PANDAS (Pediatric Autoimmune Neuropsychiatric Disorders Associated with Streptococcus), paraneoplastic cerebellar degeneration, paraneoplastic syndrome, paraneoplastic syndromes, including neurologic paraneoplastic syndromes, optionally Lambert-Eaton myasthenic syndrome or Eaton-Lambert syndrome, parasitic diseases such as Leishmania, paroxysmal nocturnal hemoglobinuria (PNH), Parry Romberg syndrome, pars planitis (peripheral uveitis), Parsonnage-Turner syndrome, parvovirus infection, pemphigoid such as pemphigoid bullous and skin pemphigoid, pemphigus (including pemphigus vulgaris), pemphigus erythematosus, pemphigus foliaceus, pemphigus mucus-membrane pemphigoid, pemphigus, peptic ulcer, periodic paralysis, peripheral neuropathy, perivenous encephalomyelitis, pernicious anemia (anemia perniciosa), pernicious anemia, phacoantigenic uveitis, pneumonocirrhosis, POEMS syndrome, polyarteritis nodosa, Type I, II, & III, polyarthritis chronica primaria, polychondritis (e.g., refractory or relapsed polychondritis), polyendocrine autoimmune disease, polyendocrine failure, polyglandular syndromes, optionally autoimmune polyglandular syndromes (or polyglandular endocrinopathy syndromes), polymyalgia rheumatica, polymyositis, polymyositis/dermatomyositis, polyneuropathies, polyradiculitis acuta, post-cardiotomy syndrome, posterior uveitis, or autoimmune uveitis, postmyocardial infarction syndrome, postpericardiotomy syndrome, post-streptococcal nephritis, post-vaccination syndromes, presenile dementia, primary biliary cirrhosis, primary hypothyroidism, primary idiopathic myxedema, primary lymphocytosis, which includes monoclonal B cell lymphocytosis, optionally benign monoclonal gammopathy and monoclonal garnmopathy of undetermined significance, MGUS, primary myxedema, primary progressive MS (PPMS), and relapsing remitting MS (RRMS), primary sclerosing cholangitis, progesterone dermatitis, progressive systemic sclerosis, proliferative arthritis, psoriasis such as plaque psoriasis, psoriasis, psoriatic arthritis, pulmonary alveolar proteinosis, pulmonary infiltration eosinophilia, pure red cell anemia or aplasia (PRCA), pure red cell aplasia, purulent or nonpurulent sinusitis, pustular psoriasis and psoriasis of the nails, pyelitis, pyoderma gangrenosum, Quervain's thyroiditis, Raynaud's phenomenon, reactive arthritis, recurrent abortion, reduction in blood pressure response, reflex sympathetic dystrophy, refractory sprue, Reiter's disease or syndrome, relapsing polychondritis, reperfusion injury of myocardial or other tissues, reperfusion injury, respiratory distress syndrome, restless legs syndrome, retinal autoimmunity, retroperitoneal fibrosis, Reynaud's syndrome, rheumatic diseases, rheumatic fever, rheumatism, rheumatoid arthritis, rheumatoid spondylitis, rubella virus infection, Sampter's syndrome, sarcoidosis, schistosomiasis, Schmidt syndrome, SCID and Epstein-Barr virus-associated diseases, sclera, scleritis, sclerodactyl, scleroderma, optionally systemic scleroderma, sclerosing cholangitis, sclerosis disseminata, sclerosis such as systemic sclerosis, sensoneural hearing loss, seronegative spondyloarthritides, Sheehan's syndrome, Shulman's syndrome, silicosis, Sjögren's syndrome, sperm & testicular autoimmunity, sphenoid sinusitis, Stevens-Johnson syndrome, stiff-man (or stiff-person) syndrome, subacute bacterial endocarditis (SBE), subacute cutaneous lupus erythematosus, sudden hearing loss, Susac's syndrome, Sydenham's chorea, sympathetic ophthalmia, systemic lupus erythematosus (SLE) or systemic lupus erythematodes, cutaneous SLE, systemic necrotizing vasculitis, ANCA-associated vasculitis, optionally Churg-Strauss vasculitis or syndrome (CSS), tabes dorsalis, Takayasu's arteritis, telangiectasia, temporal arteritis/Giant cell arteritis, thromboangiitis ubiterans, thrombocytopenia, including thrombotic thrombocytopenic purpura (TTP) and autoimmune or immune-mediated thrombocytopenia such as idiopathic thrombocytopenic purpura (ITP) including chronic or acute ITP, thrombocytopenic purpura (TTP), thyrotoxicosis, tissue injury, Tolosa-Hunt syndrome, toxic epidermal necrolysis, toxic-shock syndrome, transfusion reaction, transient hypogammaglobulinemia of infancy, transverse myelitis, traverse myelitis, tropical pulmonary eosinophilia, tuberculosis, ulcerative colitis, undifferentiated connective tissue disease (UCTD), urticaria, optionally chronic allergic urticaria and chronic idiopathic urticaria, including chronic autoimmune urticaria, uveitis, anterior uveitis, uveoretinitis, valvulitis, vascular dysfunction, vasculitis, vertebral arthritis, vesiculobullous dermatosis, vitiligo, Wegener's granulomatosis (Granulomatosis with Polyangiitis (GPA)), Wiskott-Aldrich syndrome, or x-linked hyper IgM syndrome.

Optionally, the method or use is used to treat an autoimmune disease selected from the group consisting of multiple sclerosis, psoriasis; rheumatoid arthritis; psoriatic arthritis, systemic lupus erythematosus (SLE); discoid lupus erythematosus, inflammatory bowel disease, ulcerative colitis; Crohn's disease; benign lymphocytic angiitis, thrombocytopenic purpura, idiopathic thrombocytopenia, idiopathic autoimmune hemolytic anemia, pure red cell aplasia, Sjögren's syndrome, rheumatic disease, connective tissue disease, inflammatory rheumatism, degenerative rheumatism, extra-articular rheumatism, juvenile rheumatoid arthritis, arthritis uratica, muscular rheumatism, chronic polyarthritis, cryoglobulinemic vasculitis, ANCA-associated vasculitis, antiphospholipid syndrome, myasthenia gravis, autoimmune haemolytica anemia, Guillain-Barré syndrome, chronic immune polyneuropathy, autoimmune thyroiditis, insulin dependent diabetes mellitus, type I diabetes, Addison's disease, membranous glomerulonephropathy, Goodpasture's disease, autoimmune gastritis, autoimmune atrophic gastritis, pernicious anemia, pemphigus, pemphigus vulgaris, cirrhosis, primary biliary cirrhosis, dermatomyositis, polymyositis, fibromyositis, myogelosis, celiac disease, immunoglobulin A nephropathy, Henoch-Schönlein purpura, Evans syndrome, dermatitis, atopic dermatitis, psoriasis, psoriasis arthropathica, Graves' disease, Graves' ophthalmopathy, scleroderma, systemic scleroderma, progressive systemic scleroderma, asthma, allergy, primary biliary cirrhosis, Hashimoto's thyroiditis, primary myxedema, sympathetic ophthalmia, autoimmune uveitis, hepatitis, chronic action hepatitis, collagen diseases, ankylosing spondylitis, periarthritis humeroscapularis, panarteritis nodosa, chondrocalcinosis, Wegener's granulomatosis, microscopic polyangiitis, chronic urticaria, bullous skin disorders, pemphigoid, atopic eczema, childhood autoimmune hemolytic anemia, idiopathic autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, gingivitis, periodontitis, pancreatitis, idiopathic pericarditis, myocarditis, vasculitis, gastritis, gout, gouty arthritis, and inflammatory skin disorders, normocomplementemic urticarial vasculitis, pericarditis, myositis, anti-synthetase syndrome, scleritis, macrophage activation syndrome, Behçet's Syndrome, PAPA Syndrome, Blau's Syndrome, gout, adult and juvenile Still's disease, cryropyrinopathy, Muckle-Wells syndrome, familial cold-induced auto-inflammatory syndrome, neonatal onset multisystemic inflammatory disease, familial Mediterranean fever, chronic infantile neurologic, cutaneous and articular syndrome, a rheumatic disease, polymyalgia rheumatica, mixed connective tissue disease, inflammatory rheumatism, degenerative rheumatism, extra-articular rheumatism, juvenile arthritis, juvenile rheumatoid arthritis, systemic juvenile idiopathic arthritis, arthritis uratica, muscular rheumatism, chronic polyarthritis, reactive arthritis, Reiter's syndrome, rheumatic fever, relapsing polychondritis, Raynaud's phenomenon, vasculitis, cryoglobulinemic vasculitis, temporal arteritis, giant cell arteritis, Takayasu arteritis, Behcet's disease, chronic inflammatory demyelinating polyneuropathy, autoimmune thyroiditis, insulin dependent diabetes mellitus, type I diabetes, Addison's disease, membranous glomerulonephropathy, polyglandular autoimmune syndromes, Goodpasture's disease, autoimmune gastritis, autoimmune atrophic gastritis, pernicious anemia, pemphigus, pemphigus vulgaris, cirrhosis, primary biliary cirrhosis, idiopathic pulmonary fibrosis, myositis, dermatomyositis, juvenile dermatomyositis, polymyositis, fibromyositis, myogelosis, celiac disease, celiac sprue dermatitis, immunoglobulin A nephropathy, Henoch-Schonlein purpura, Evans syndrome, atopic dermatitis, psoriasis, psoriasis vulgaris, psoriasis arthropathica, Graves' disease, Graves' ophthalmopathy, scleroderma, systemic scleroderma, progressive systemic scleroderma, diffuse scleroderma, localized scleroderma, Crest syndrome, asthma, allergic asthma, allergy, primary biliary cirrhosis, fibromyalgia, chronic fatigue and immune dysfunction syndrome (CFIDS), autoimmune inner ear disease, Hyper IgD syndrome, Schnitzler's syndrome, autoimmune retinopathy, age-related macular degeneration, atherosclerosis, chronic prostatitis, alopecia, alopecia areata, alopecia universalis, alopecia totalis, autoimmune thrombocytopenic purpura, idiopathic thrombocytopenic purpura, pure red cell aplasia, and TNF receptor-associated periodic syndrome (TRAPS).

Optionally the diagnosis and/or treatment is combined with another moiety useful for treating immune related condition.

Optionally said other moiety useful for treating immune related condition is selected from immunosuppressants such as corticosteroids, cyclosporin, cyclophosphamide, prednisone, azathioprine, methotrexate, rapamycin, tacrolimus, leflunomide or an analog thereof; mizoribine; mycophenolic acid; mycophenolate mofetil; 15-deoxyspergualine or an analog thereof; biological agents such as TNF-α blockers or antagonists, or any other biological agent targeting any inflammatory cytokine, nonsteroidal antiinflammatory drugs/Cox-2 inhibitors, hydroxychloroquine, sulphasalazopryine, gold salts, etanercept, infliximab, mycophenolate mofetil, basiliximab, atacicept, rituximab, cytoxan, interferon β-1a, interferon β-1b, glatiramer acetate, mitoxantrone hydrochloride, anakinra and/or other biologics and/or intravenous immunoglobulin (IVIG), interferons such as IFN-β-1a (REBIF®. AVONEX® and CINNOVEX®) and IFN-β-1b (BETASERON®); EXTAVIA®, BETAFERON®, ZIFERON®); glatiramer acetate (COPAXONE®), a polypeptide; natalizumab (TYSABRI®), mitoxantrone (NOVANTRONE®), a cytotoxic agent, a calcineurin inhibitor, e.g. cyclosporin A or FK506; an immunosuppressive macrolide, e.g. rapamycine or a derivative thereof; e.g. 40-O-(2-hydroxy)ethyl-rapamycin, a lymphocyte homing agent, e.g. FTY720 or an analog thereof, corticosteroids; cyclophosphamide; azathioprene; methotrexate; leflunomide or an analog thereof; mizoribine; mycophenolic acid; mycophenolate mofetil; 15-deoxyspergualine or an analog thereof; immunosuppressive monoclonal antibodies, e.g., monoclonal antibodies to leukocyte receptors, e.g., MHC, CD2, CD3, CD4, CD11a/CD18, CD7, CD25, CD27, B7, CD40, CD45, CD58, CD137, ICOS, CD150 (SLAM), OX40, 4-1BB or their ligands; or other immunomodulatory compounds, e.g. CTLA4-Ig (abatacept, ORENCIA®, belatacept), CD28-Ig, B7-H4-Ig, or other costimulatory agents, or adhesion molecule inhibitors, e.g. mAbs or low molecular weight inhibitors including LFA-1 antagonists, Selectin antagonists and VLA-4 antagonists, or another immunomodulatory agent.

According to at least some embodiments, there is provided an anti-VSTM5 antibody or antigen-binding fragment or composition, or method or use according to any of the foregoing claims which includes another moiety is useful for reducing the undesirable immune activation that follows gene therapy.

Optionally the anti-VSTM5 antibody or antigen-binding fragment or composition, or method or use according to any of the foregoing or as described herein includes treatment with an anti-VSTM5 antibody or antigen-binding fragment or composition containing combined with another therapeutic agent or therapy.

Optionally the anti-VSTM5 antibody or antigen-binding fragment or composition, or method or use according to any of the foregoing or as described herein further comprises a Therapeutic agent targeting immunosuppressive cells Tregs and/or MDSCs is selected from antimitotic drugs, cyclophosphamide, gemcitabine, mitoxantrone, fludarabine, thalidomide, thalidomide derivatives, COX-2 inhibitors, depleting or killing antibodies that directly target Tregs through recognition of Treg cell surface receptors, anti-CD25 daclizumab, basiliximab, ligand-directed toxins, denileukin diftitox (Ontak)—a fusion protein of human IL-2 and diphtheria toxin, or LMB-2—a fusion between an scFv against CD25 and the pseudomonas exotoxin, antibodies targeting Treg cell surface receptors, TLR modulators, agents that interfere with the adenosinergic pathway, ectonucleotidase inhibitors, or inhibitors of the A2A adenosine receptor, TGF-β inhibitors, chemokine receptor inhibitors, retinoic acid, all-trans retinoic acid (ATRA), Vitamin D3, phosphodiesterase 5 inhibitors, sildenafil, ROS inhibitors and nitroaspirin.

Optionally the anti-VSTM5 antibody or antigen-binding fragment or composition, or method or use according to any of the foregoing or as described herein further comprises another antibody is selected from antagonistic antibodies targeting one or more of CTLA4, PD-1, PDL-1, LAG-3, TIM-3, BTLA, B7-H4, B7-H3, VISTA, and/or Agonistic antibodies targeting one or more of CD40, CD137, OX40, GITR, CD27, CD28 or ICOS.

Optionally the method or use includes assaying VSTM5 protein by the individual's cells prior, concurrent and/or after treatment.

Optionally the method detects the expression of VSTM5 protein by diseased and/or normal cells prior to treatment, optionally by the use of an antibody or nucleic acid that detects VSTM5 expression.

Optionally the method or use further includes the administration or use of another diagnostic or therapeutic agent, which may be administered prior, concurrent or after the administration of the anti-VSTM5 antibody, or antigen-binding fragment or composition containing such according to any of the foregoing or as described herein.

Optionally the method or use further includes the administration of another therapeutic agent.

Optionally the other therapeutic agent is selected from a drug, another immunomodulatory compound, a radionuclide, a fluorophore, an enzyme, a toxin, or a chemotherapeutic agent; and the detectable agent is selected from a radioisotope, a metal chelator, an enzyme, a fluorescent compound, a bioluminescent compound or a chemiluminescent compound.

Optionally the method or use further includes the administration of an antibody or antigen-binding fragment thereof which specifically binds to a NK cell receptor.

Optionally the antibody or antigen-binding fragment thereof which specifically binds to an NK cell receptor agonizes the effect of said NK cell receptor.

Optionally the antibody or antigen-binding fragment thereof which specifically binds to an NK cell receptor antagonizes the effect of said NK cell receptor.

Optionally the NK cell receptor is one that inhibits NK cell activity.

Optionally the inhibitory NK cell receptor is selected from the group consisting of KIR2DL1, KIR2DL2/3, KIR2DL4, KIR2DL5A, KIR2DL5B, KIR3DL1, KIR3DL2, KIR3DL3, LILRB1, NKG2A, NKG2C, NKG2E and LILRBS.

Optionally the NK cell receptor is one that promotes NK cell activity.

Optionally the NK cell activating receptor is selected from the group consisting of NKp30, NKp44, NKp46, NKp46, NKG2D, KIR2DS4 CD2, CD16, CD69, DNAX accessory molecule-1 (DNAM-1), 2B4, NK1.1; a killer immunoglobulin (Ig)-like activating receptors (KAR); ILTs/LIRs; NKRP-1, CD69; CD94/NKG2C and CD94/NKG2E heterodimers, NKG2D homodimer KIR2DS and KIR3DS.

According to at least some embodiments, there is provided an assay method for selecting an anti-VSTM5 antibody or antigen-fragment according to any of the foregoing claims, or an anti-VSTM5 antibody or antigen-fragment suitable for use in a method or use according to any of the foregoing claims, wherein the method comprises (i) obtaining one or more antibodies that putatively bind to a VSTM5 polypeptide having a sequence selected from an amino acid sequence set forth in any of SEQ ID NOs:1, 2, 3, 6, 7 or 12-21, 349, or binding to a polypeptide possessing at least 90% sequence identity therewith or to a non-human VSTM5 ortholog, or a fragment or variant thereof containing at least one VSTM5 epitope, which fragment or variant possesses at least 90% identity thereto, or to a non-human VSTM5 ortholog (ii) determining whether said antibody or antigen-binding fragment specifically binds to said VSTM5 polypeptide, (ii) determining whether said antibody or antigen-binding fragment modulates (agonizes or antagonizes) at least one effect of VSTM5 on immunity, and (iv) if (ii) and (ii) are satisfied selecting said antibody as one potentially useful in a method or use according to any of the foregoing or as described.

Optionally the method further includes humanization, primatization or chimerization if the antibody or antigen-binding fragment is not a human or non-human primate antibody or a fragment thereof.

Optionally the immunogen used to derive said antibody or antigen-binding fragment comprises a VSTM5 polypeptide having a sequence selected from an amino acid sequence set forth in any of SEQ ID NOs:1, 2, 3, 6, 7 or 12-21, 132, 349, or binding to a polypeptide possessing at least 90% sequence identity therewith or to a non-human VSTM5 ortholog or the same region of a nn-human VSTM5 ortholog, or a fragment or variant thereof containing at least one VSTM5 epitope.

Optionally the immunogen used to derive said antibody or antigen-binding fragment comprises a VSTM5 polypeptide having a sequence selected from an amino acid sequence set forth in any of SEQ ID NOs:1, 2, 3, 6, 7 or 12-21, 132, 349, or binding to a polypeptide possessing at least 90% sequence identity therewith or to the same region of a non-human ortholog of hVSTM5.

Optionally the immunogen used to derive said antibody or antigen-binding fragment thereof consists of a polypeptide having an amino acid sequence set forth in any of SEQ ID NOs:1, 12-21, or binding to a polypeptide possessing at least 90% sequence identity therewith or to the same region of a non-human VSTM5 ortholog, or a conjugate thereof not containing another portion of any of the VSTM5 polypeptide.

Optionally the selected antibody or antigen-binding fragment thereof specifically binds to a first polypeptide having an amino acid sequence set forth in any of SEQ ID NOs:1, 12-21, or binds to a polypeptide possessing at least 90% sequence identity therewith or to the same region of a non-human VSTM5 ortholog, which first polypeptide is contained in a second polypeptide having an amino acid sequence set forth in any of SEQ ID NOs: 2, 3, 6, 7, 132, 349, or in a polypeptide possessing at least 90% sequence identity with said second polypeptide having an amino acid sequence set forth in any of SEQ ID NOs: 2, 3, 6, 7, 132, 349 or to a non-human VSTM5 ortholog of said second polypeptide having an amino acid sequence set forth in any of SEQ ID NOs: 2, 3, 6, 7, 132, 349 and said antibody or antigen-binding region does not specifically bind to any other portion of said second polypeptide apart from said first polypeptide.

Optionally the assay uses hybridomas, cell lines, B cells or a phage or a yeast antibody library which produce said putative anti-VSTM5 antibody or antigen-binding fragment, or a composition comprising isolated putative anti-VSTM5 antibodies.

Optionally step (iii) detects whether the anti-VSTM5 antibody or antigen binding fragment antagonizes at least one effect of VSTM5 on immunity.

Optionally step (iii) detects whether the anti-VSTM5 antibody or antigen binding fragment agonizes at least one effect of VSTM5 on immunity.

Optionally the selected antibody is demonstrated to mediate at least one of the following effects: (i) increases immune response, (ii) increases T cell activation, (iii) increases cytotoxic T cell activity, (iv) increases NK cell activity, (v) alleviates T-cell suppression, (vi) increases pro-inflammatory cytokine secretion, (vii) increases IL-2 secretion; (viii) increases interferon-γ production, (ix) increases Th1 response, (x) decrease Th2 response, (xi) decreases or eliminates cell number and/or activity of at least one of regulatory T cells (Tregs), myeloid derived suppressor cells (MDSCs), iMCs, mesenchymal stromal cells, TIE2-expressing monocytes, (xii) reduces regulatory cell activity, and/or the activity of one or more of myeloid derived suppressor cells (MDSCs), iMCs, mesenchymal stromal cells, TIE2-expressing monocytes, (xiii) decreases or eliminates M2 macrophages, (xiv) reduces M2 macrophage pro-tumorigenic activity, (xv) decreases or eliminates N2 neutrophils, (xvi) reduces N2 neutrophils pro-tumorigenic activity, (xvii) reduces inhibition of T cell activation, (xviii) reduces inhibition of CTL activation, (xix) reduces inhibition of NK cell activation, (xx) reverses T cell exhaustion, (xxi) increases T cell response, (xxii) increases activity of cytotoxic cells, (xxiii) stimulates antigen-specific memory responses, (xxiv) elicits apoptosis or lysis of cancer cells, (xxv) stimulates cytotoxic or cytostatic effect on cancer cells, (xxvi) induces direct killing of cancer cells, (xxvii) increases Th17 activity and/or (xxviii) induces complement dependent cytotoxicity and/or antibody dependent cell-mediated cytotoxicity, with the proviso that said anti-VSTM5 antibody or antigen-binding fragment may elicit an opposite effect to one or more of (i)-(xxviii).

Optionally the selected antibody is demonstrated to mediate at least one of the following effects: (i) decreases immune response, (ii) decreases T cell activation, (iii) decreases cytotoxic T cell activity, (iv) decreases natural killer (NK) cell activity, (v) decreases T-cell activity, (vi) decreases pro-inflammatory cytokine secretion, (vii) decreases IL-2 secretion; (viii) decreases interferon-γ production, (ix) decreases Th1 response, (x) decreases Th2 response, (xi) increases cell number and/or activity of regulatory T cells, (xii) increases regulatory cell activity and/or one or more of myeloid derived suppressor cells (MDSCs), iMCs, mesenchymal stromal cells, TIE2-expressing monocytes, (xiii) increases regulatory cell activity and/or the activity of one or more of myeloid derived suppressor cells (MDSCs), iMCs, mesenchymal stromal cells, TIE2-expressing monocytes, (xiii) increases M2 macrophages, (xiv) increases M2 macrophage activity, (xv) increases N2 neutrophils, (xvi) increases N2 neutrophils activity, (xvii) increases inhibition of T cell activation, (xviii) increases inhibition of CTL activation, (xix) increases inhibition of NK cell activation, (xx) increases T cell exhaustion, (xxi) decreases T cell response, (xxii) decreases activity of cytotoxic cells, (xxiii) reduces antigen-specific memory responses, (xxiv) inhibits apoptosis or lysis of cells, (xxv) decreases cytotoxic or cytostatic effect on cells, (xxvi) reduces direct killing of cells, (xxvii) decreases Th17 activity, and/or (xxviii) reduces complement dependent cytotoxicity and/or antibody dependent cell-mediated cytotoxicity, with the proviso that said anti-VSTM5 antibody or antigen-binding fragment may elicit an opposite effect to one or more of (i)-(xxviii).

Optionally the selected antibody agonizes or antagonizes the effects of VSTM5 on T cell activity, NK cell activity, and/or the production of one or more proinflammatory cytokines.

Optionally the selected antibody is demonstrated to compete with binding to human or rodent VSTM5 as an anti-VSTM5 antibodies according to any one of the foregoing as described herein.

According to at least some embodiments, there is provided an immunomodulatory antibody or antigen-binding obtained according to any of the foregoing or as described herein, or a pharmaceutical or diagnostic composition containing same.

Optionally the immunomodulatory antibody or antigen-binding or a pharmaceutical or diagnostic composition containing same is provided for treating or diagnosing a disease selected from cancer, infection, sepsis, autoimmunity, inflammation, allergic or other immune condition or to suppress an undesired immune reaction to a cell or gene therapy therapeutic or a transplanted cell, tissue or organ.

According to at least some embodiments, there is provided a transplant therapy which includes the transplant of cells, tissue or organ into a recipient, wherein the cells, tissue or organ or treated ex vivo using a composition containing an anti-VSTM5 antibody or antigen-binding fragment or composition according to any of the foregoing or as described herein, prior to infusion or transplant of said cells, tissue or organ into the recipient.

Optionally the composition comprises immune cells of the donor and/or transplant recipient.

Optionally the transplanted cells, tissue or organ comprises bone marrow, other lymphoid cells or tissue or stem cells.

According to at least some embodiments, there is provided a nucleic acid encoding the variable heavy and/or light region polypeptide of an anti-VSTM5 antibody or antibody fragment according to any of the foregoing or as described herein.

According to at least some embodiments, there is provided a nucleic acid encoding an antibody heavy and/or light variable region of an anti-VSTM5 antibody, wherein said nucleic acid possesses at least 90, 95, 96, 97, 98 or 99% sequence identity to the variable heavy or light coding region of a nucleic acid selected from those in SEQ ID NO:157-180.

According to at least some embodiments, there is provided a nucleic acid encoding an antibody heavy variable region of an anti-VSTM5 antibody, wherein said nucleic acid possesses at least 90, 95, 96, 97, 98 or 99% sequence identity to the variable heavy coding region of a nucleic acid selected from those in SEQ ID NO:157, 159, 161, 163, 165, 167, 169, 171, 173, 175, 177 and 179.

According to at least some embodiments, there is provided a nucleic acid encoding an antibody light variable region of an anti-VSTM5 antibody, wherein said nucleic acid possesses at least 90, 95, 96, 97, 98 or 99% sequence identity to the variable light coding region of a nucleic acid selected from those in SEQ ID NO:158, 160, 162, 164, 166, 168, 170, 172, 174, 176, 178 and 180.

According to at least some embodiments, there is provided a nucleic acid encoding the variable heavy and/or light regions of an anti-VSTM5 antibody, wherein said nucleic acid contains a sequence which is identical to any one of SEQ ID NO:157-180.

According to at least some embodiments, there is provided a nucleic acid encoding the variable heavy and light regions of an anti-VSTM5 antibody, wherein said nucleic acid contains a nucleic acid encoding an antibody heavy variable region of an anti-VSTM5 antibody, wherein said nucleic acid possesses at least 90, 95, 96, 97, 98 or 99% sequence identity to the variable heavy coding region of a nucleic acid selected from those in SEQ ID NO:157, 159, 161, 163, 165, 167, 169, 171, 173, 175, 177 and 179 and further comprises a nucleic acid encoding an antibody light variable region of an anti-VSTM5 antibody, wherein said nucleic acid possesses at least 90, 95, 96, 97, 98 or 99% sequence identity to the variable light coding region of a nucleic acid selected from those in SEQ ID NO:158, 160, 162, 164, 166, 168, 170, 172, 174, 176, 178 and 180.

Optionally the nucleic acid is operably linked to a promoter which is constitutive or inducible.

Optionally the nucleic acid is attached to a nucleic acid encoding an antibody constant domain or fragment thereof which optionally may be mutated to alter (increase or decrease) effector function or Fab arm exchange.

Optionally the constant region is a human IgG1, IgG2, IgG3 or IgG4 constant region which optionally may be mutated to alter (increase or decrease) effector function or Fab arm exchange.

Optionally 1, 2 or all 3 of the CDRs of the variable heavy polypeptide and/or 1, 2 or all 3 of the CDRs of the encoded variable light polypeptide encoded by said nucleic acid are respectively identical to those of a variable heavy region encoded by one of the nucleic acids of SEQ ID NO:157, 159, 161, 163, 165, 167, 169, 171, 173, 175, 177 and 179 and/or to those of a variable light region encoded by one of the nucleic acids of SEQ ID NO:158, 160, 162, 164, 166, 168, 170, 172, 174, 176, 178 and 180.

According to at least some embodiments, there is provided a vector or virus comprising at least one nucleic acid according to any of the foregoing or as described herein.

According to at least some embodiments, there is provided an isolated or recombinant cell which comprises at least one nucleic acid or vector or virus according to any of the foregoing or as described herein.

Optionally the cell is selected from a hybridoma and a recombinant bacterial, yeast or fungal, mammalian, insect, amphibian, reptilian, plant, and avian cell or egg.

Optionally the cell is a yeast or mammalian cell.

Optionally the cell is human or rodent.

According to at least some embodiments, there is provided a method of producing an anti-VSTM5 antibody or antibody fragment by culturing an isolated or recombinant cell according to any of the foregoing or as described herein. Optionally the cell used in the method is a bacterial, yeast, fungal, insect, plant, reptilian, mammalian cell or an avian egg. Optionally the cell used in the method is a yeast or mammalian cell.

Optionally the cell used in the method is human or murine.

The present invention according to at least some embodiments relates to antibodies and antigen-binding fragments that bind to VSTM5, preferably those that modulate at least one effect of VSTM5 on immunity. “VSTM5” or “V-Set And Transmembrane Domain-Containing Protein 5” is described by Taylor et al., “Human chromosome 11 DNA sequence and analysis including novel gene identification”, Nature 440, 497-500 (2006). Taylor discloses a DNA sequence encoding a polypeptide 100% identical to the VSTM5 amino acid sequence (SEQ ID NO:6). The reference does not characterize the activity of this protein or more specifically its immunosuppressive effects on T cell and NK immunity.

US patent application number US20080299042, assigned to Biogen Idec, Inc., discloses sequences of numerous nucleic acid molecules that encode membrane associated proteins, the proteins themselves, and antibodies to the proteins. Also disclosed are methods of treating cancer and autoimmune diseases, specifically referencing colon cancer, lung cancer, pancreatic cancer and ovarian cancer. Included in the application is sequence SEQ ID NO: 1709, which is a sequence identical at 155 of 186 amino acid residues to the VSTM5 amino acid sequence. The reference does not characterize the activity of this protein or more specifically its immunosuppressive effects on T cell or NK cell immunity.

PCT application WO2003025148 assigned to Hyseq, discloses SEQ ID NO 332, which is identical to the wild type VSTM5. The '148 application states that the disclosed polypeptides are useful for raising antibodies, as markers for tissues in which the corresponding polypeptide is expressed, for re-engineering damaged or diseased tissues, for treating myeloid or lymphoid cell disorders, in bone cartilage, tendon, ligament and/or nerve tissue growth or regeneration, in wound healing, in tissue repair and replacement, in healing of burns, incisions and ulcers, and in treating cancer. The reference does not characterize the activity of this protein or more specifically its immunosuppressive effects on T cell or NK immunity.

PCT Application No: PCT/US2008/075122, owned in common with the present application, discloses the VSTM5 protein and is identified in this application as Sequence 43, which further corresponds to residues 29-147 of the sequence referred to in this application as AI216611_P0. This PCT application teaches that AI216611 and other proteins are differentially expressed by some cancers, and further suggests their potential use as targets and specifically for obtaining antibodies for potential use in immunotherapy, cancer therapy, and drug development. The reference states that these polypeptides possess a B7-like structure and may be costimulatory molecules. Anti-VSTM5 antibodies and use thereof are prophetically disclosed.

Also, the above referenced publication, patents and/or patent applications do not teach or suggest an antibody or an antigen-binding fragment thereof, said antibody having an antigen-binding region that binds specifically to a first polypeptide having an amino acid sequence set forth in any of SEQ ID NOs:1, 12-21, wherein a second polypeptide having an amino acid sequence set forth in any of SEQ ID NOs: 2, 3, 6, 7, 132, 349 comprises said first polypeptide, with the proviso that said antigen-binding region does not specifically bind to any other portion of said second polypeptide apart from said first polypeptide.

Furthermore, the above referenced publication, patents and/or patent applications do not teach the use of antibodies specific to the VSTM5 ECD for the treatment and/or diagnosis of specific cancers as described herein. Furthermore, the above referenced publication, patents and/or patent applications do not teach the use of antibodies specific to the VSTM5 ECD for cancer immunotherapy, wherein the cancer does not express VSTM5 proteins at diagnosis or prior to combination therapy with other therapeutic agents for cancer treatment, as described herein.

### BRIEF DESCRIPTION OF THE FIGURES

FIG. 1 contains the complete clinical profiles of multitumor tissue microarray samples analyzed for VSTM5 expression (See Example 1).

FIG. 2 contains the complete clinical profiles of full section lymphoid tissue samples analyzed for VSTM5 expression (See Example 1).

FIG. 3 contains the tissue description of the “TOP4” tissues which comprise triplicate tissue core samples from 120 patients analyzed for VSTM5 expression. (See Example 1).

FIG. 4 contains the immunoreactivity results (IHC values or scores) for individual samples assayed for VSTM5 expression (multitumor tissue arrays of FIG. 1). (See Example 1).

FIG. 5 contains a summary of the IHC values or scores of the TOP4 tissue microarray samples. (See Example 1).

FIG. 6 Schematic presentation of elevation of endogenous expression of the immune checkpoint ligand (PDL-1) by induction of anti-tumor immunity.

FIG. 7 presents the results of the western blot analysis of ectopically expressed human VSTM5 proteins using an anti-VSTM5 antibody, described in details in Example 2 herein. Whole cell extracts (30 ug) of HEK293T cell pools, previously transfected with expression construct encoding human VSTM5 (lane 1), empty vector (lane 2) or with expression construct encoding human VSTM5-EGFP (lane 3), were analyzed by WB using an anti-VSTM5 antibody.

FIG. 8 presents the results of cell surface expression of mouse VSTM5, human VSTM5 and VSTM5-EGFP proteins by FACS analysis, described in details in Example 2 herein. The anti-VSTM5 mAb (10 ug/ml) (FIGS. 8A and 8B for human VSTM5 and VSTM5-EGFP, respectively), or monoclonal VSTM5 Ab (S53-01-B11) (FIGS. 8C and 8D) were used to analyze HEK-293T cells stably expressing the VSTM5 proteins. In FIGS. 8A and 8B rabbit IgG was used as Isotype control to the pAb. Cells expressing the empty vector (pRp=pIRESpuro3) were used as negative control. Detection was carried out by donkey anti-rabbit FITC or PE-conjugated secondary Ab and analyzed by FACS. FIGS. 8C and 8D demonstrate membrane expression of human VSTM5 protein and mouse VSTM5 protein, respectively, by using 1 nM (0.15 ug/ml) monoclonal VSTM5 Ab (553-01-B11) compared to 1 nM (0.15 ug/ml) IgG1 control antibody followed by PE-Goat a human secondary conjugated Ab in 1:200 dilution and analyzed by Flow Cytometry. Non expressing cell line (HEK293T_pIRESpuro3) was stained under the same conditions and used for a negative control.

FIG. 9 presents a schematic illustration of the experimental setting of an in-vitro co-culture assay testing the effect of VSTM5, expressed on HEK 293T cells, on the activation of Jurkat cells by plate bound anti-CD3, as described in Example 3 herein.

FIG. 10 demonstrates that VSTM5_GFP (SEQ ID NO:133) expressed on HEK-293T cells inhibits Jurkat cells activation, as described in details in Example 3 herein. HEK-293T cells expressing VSTM5_GFP (SEQ ID NO:133) (293T-VSTM5) or the empty vector (293T-pRp) were seeded at 25,000 (A) or 50,000 (B) cells per well, in wells pre-coated with 2 μg/ml of anti-CD3. Jurkat cells were added 2 hours later at 50,000 cells per well, and the co-cultures were incubated O.N. Cells were analyzed for the expression of CD69 by flow cytometry. As reference, CD69 values of untreated Jurkat cells, i.e. not treated with anti-CD3, are shown. ΔMFI values of CD69 between untreated and anti-CD3 treated Jurkat cells in the presence of 25,000 or 50,000 HEK-293 transfected cells per well are presented in (C). The percentage of inhibition of Jurkat cells activation in the presence of 293T-VSTM5 cells is presented in (D). * indicates value significantly different from that of the empty vector (p<0.05, Student's t-test).

FIG. 11 presents VSTM5-ECD-Ig suppression of CD4 T cell activation, described in details in Example 4 herein. (A-B) CD4⁺CD25⁻CD62L⁺ T cells (1×10⁵ per well) were stimulated with plate bound anti-CD3 mAb (2 μg/ml) in the presence of 2, 4 or 8 μg/ml of VSTM5-ECD-Ig H:M (SEQ ID NO: 131) or control Ig (i.e. 1:1, 1:2, 1:4 anti-CD3: tested protein ratio, respectively). Culture supernatants were collected at 48 hrs post-stimulation and mouse IL-2 or IFNγ levels were analyzed by ELISA. Results are shown as Mean±Standard errors of triplicate samples. (C) CFSE-labeled CD4⁺CD25⁻ cells were stimulated for 72 h with immobilized anti-CD3 mAb (0.5 μg/ml) in the presence of 0.5 or 1 ug/ml of VSTM5-ECD-Ig H:M or control Ig (1:1, 1:2 anti-CD3: tested protein ratio, respectively). M1 marker refers to the fraction of dividing cells (CFSE^{low}), presented in the histograms as % CFSE^{low} CD4 T cells. (D) CD4⁺CD25⁻ T cells (1×10⁵ per well) were stimulated with immobilized anti-CD3 mAb (2 μg/ml) in the presence of 10 ug/ml of VSTM5-ECD-Ig M:M (SEQ ID NO: 8) or control Ig, or in the absence of additional proteins (PBS). The expression of CD69 was analyzed by flow cytometry at 48 h post-stimulation.

FIG. 12 demonstrates that VSTM5 ECD-Ig (SEQ ID NO: 130) inhibits human T cell proliferation induced by anti-CD3 and anti-CD28 in the presence of irradiated autologous PBMCs, as described in details in Example 5 herein. 1.5×10⁵ naïve CD4⁺ T cells were activated with anti-CD3 (0.5 mg/ml), anti-CD28 (0.5 mg/ml) in the presence of 1.5×10⁵ irradiated autologous PBMCs. VSTM5-ECD-Ig or hIgG1 control Ig (Synagis®) was added to the culture at the indicated concentrations. Proliferation was evaluated using H3-tymidine incorporation at 72 hours. Shown are averages of three donors tested.

FIG. 13 demonstrates that VSTM5-ECD-Ig H:H (SEQ ID NOs: 130) and VSTM5-ECD-Ig M:M (SEQ ID NOs: 8) bind H9. (A) H9 cells were incubated with a dose titration of VSTM5-ECD-Ig H:H or control human IgG1. (B) H9 cells were incubated with a dose titration of VSTM5-ECD-Ig M:M or control mouse IgG2a (Mopc173). Binding was detected by FACS analysis following the three-step detection protocol, described in Example 6 herein.

FIG. 14 demonstrates that binding of biotinylated VSTM5-ECD-Ig to H9 cells can be competed off with unlabeled VSTM5-ECD-Ig in a dose dependent manner (A) H9 cells were incubated with a dose titration of biotinylated VSTM5-ECD-Ig H:H. Binding was detected by FACS analysis following the two-step detection protocol (VSTM5; human IgG1 control). (B) Unlabeled VSTM5-ECD-Ig H:H or human IgG1 isotype control (ET901) was incubated with H9 cells prior to binding with 44 nM biotinylated VSTM5-ECD-Ig H:H, as described in the competition assay protocol in Example 6 herein.

FIG. 15 contains the gating strategy used for flow cytometry analysis of VSTM5 expression on resting and activated T cells, as described in Example 7 herein.

FIGS. 16(A) and (B) contain the results of experiments showing the binding of unlabeled VSTM5-ECD-Ig fusion protein to anti-CD3 activated, but not resting, human CD4⁺ T cells, as described in Example 7 herein. B7-H1-Ig and Synagis (hIgG1) were used as positive and negative controls, respectively.

FIGS. 17(A) and (B) contain the results of experiments showing the binding of unlabeled VSTM5-ECD-Ig fusion protein to anti-CD3 activated, but not resting, human CD8⁺ T cells, as described in Example 7 herein. B7-H1-Ig and Synagis (hIgG1) were used as positive and negative controls, respectively.

FIG. 18 shows that VSTM5-ECD-Ig M:M (SEQ ID NO:8) enhances iTreg cell differentiation. CD4⁺CD25⁻ T cells were activated for 4 days in 96 well plates using immobilized anti-CD3 (5 μg/ml) and soluble anti-CD28 (1 μg/ml) in the presence of purified CD11c⁺ dendritic cells (APCs) at a 1:5 cell ratio. Soluble VSTM5-ECD-Ig M:M (SEQ ID NO:8) was added at 10 μg/ml. Cultures were treated with iTreg driving conditions, i.e. TGFβ (5 ng/ml) and mIL-2 (5 ng/ml). Development of Foxp3⁺CD4⁺ iTreg cells was assessed by flow cytometry.

FIG. 19 shows that VSTM5-ECD-Ig M:M (SEQ ID NO: 8) enhances iTreg cell differentiation in the presence of TGF-β and IL-2. CD4⁺CD25⁻ T cells were cultured for 5 days with immobilized anti-CD3 (2 ug/ml) together with VSTM5-ECD-Ig M:M (SEQ ID NO: 8) or mIgG2a control (MOPC-173, Biolegend) at 10 μg/ml in the presence or absence of TGFβ (10 ng/ml), with or without IL-2 (5 ng/ml). Development of Foxp3⁺CD25⁺ iTreg cells was assessed by flow cytometric analysis. FIG. 19A presents representative plots of gated CD4⁺ cells. Values shown within dot plots represent the percentage of CD25⁺Foxp3⁺ of total CD4⁺ cells or total Tregs cell count/μl. FIG. 19B shows average percentage or total iTregs counts from triplicate cultures for each condition.

FIG. 20 shows that VSTM5-ECD fused to Fc of human IgG1 (SEQ ID NO:130) binds to primary activated NK cells. Human NK cell clones from one donor were incubated with 5 μg unlabeled VSTM5 (green line) or control isotype hIgG1 (grey area). Examples of high binding NK clones are shown in (A), and examples of low binding NK clones in (B).

FIG. 21 shows the over expression of VSTM5 by different cancer cell lines. Human cancer cell lines were transduced with a lentiviral expression vector encoding only DSRED (red fluorescent protein) or also VSTM5 (SEQ ID NO:132) and were evaluated by FACS analysis using a commercial rabbit polyclonal antibody and rabbit IgG as isotype control, and evaluated with an anti-rabbit secondary antibody.

FIG. 22 shows that VSTM5 over expression on cancer cell lines reduces their susceptibility to NK cells cytotoxic activity. Human polyclonal NK cells were co-incubated with human cancer cell lines (HeLa—FIG. 22A, RKO—FIG. 22B, 8866—FIG. 22C and BJAB—FIG. 22D) over expressing VSTM5 (SEQ ID NO:132) or transfected with empty vector (dsred) as negative control, and percentage of cell killing was assessed. The Y axis shows % killing. The X axis shows effector to target cells (E: T) ratios (two fold serial dilutions of effector cells), that range from 40:1 to 5:1 in the experiments with HeLa and RKO, and 30:1 to 15:1 in the experiments with BJAB and 8866. * P value<0.05, ** P value<0.02, *** P value<0.01

FIG. 23 presents a schematic illustration of the experimental system used in Example 9 herein.

FIG. 24 presents the results of FACS analysis performed on VSTM5 transduced melanoma cells SK-mel-23, mel-624, mel-624.38 and mel-888 using a specific polyclonal antibody that recognizes VSTM5, in order to assess the levels of membrane expression of this protein. The percent of cells expressing the VSTM5 protein is provided for each cell line.

FIG. 25 presents the results of FACS analysis performed on TCR F4 transduced stimulated CD8⁺ cells (CTLs) using a specific monoclonal antibody that recognizes the extracellular domain of the β-chain from the transduced F4 TCR, specific for the MART1 melanoma antigen, in order to assess the levels of membrane expression of this specific TCR.

FIG. 26A shows the effect of VSTM5 expressed on melanoma cell lines (SK-mel-23, mel-624 and mel-624.38) on the activation of F4 TCR expressing CTLs in a co-culture assay, as observed by IFNγ secretion. Mel-888 cells were used as negative control for F4 TCR-specific activation, since these cells do not express HLA-A2 and are thus not recognized by the F4TCR. The graphs show two independent experiments with CTLs from different donors transduced with F4 TCR. *p=0.01.

FIG. 26B presents a summary of several experiments using three melanoma cell lines (SK-mel-23, mel-624 and mel-624.38) overexpressing VSTM5, in a co-culture assay to evaluate the effect on activation of F4 TCR expressing CTLs. The dots represent the level of IFNγ secretion obtained in independent experiments, whereby 100% is defined as the level of secretion using the respective melanoma cell line transduced with empty vector. The left panels show results using cells with relatively low expression of VSTM5, the right panels show results using cells with relatively high expression of VSTM5.

FIG. 26C shows the effect of VSTM5 expressed on melanoma cell lines on IL-2 secretion from activated F4 TCR expressing CTLs in a co-culture assay. The graphs show two independent experiments with F4 TCR transduced CTLs from different donors. *p=0.01.

FIG. 26D shows the effect of VSTM5 expressed on melanoma cells on reduction of TNFα secretion from F4 TCR expressing CTLs in a co-culture assay. The graph shows one experiment with F4 TCR transduced CTLs from one donor. p=0.01.

FIG. 27 demonstrates the susceptibility of mel-624 melanoma cell lines overexpressing VSTM5 or transfected with empty vector, to killing by F4 transduced or non-transduced (‘w/o’) lymphocytes from one donor. The Effector to Target ratio was 1:1 or 1:3. Percentages are of double positive cells stained for CFSE and PI, and indicate level of cell killing.

FIG. 28 contains the results of binding assays wherein beads were coated with 50 ug/ml of anti-CD3 mAb and different concentrations of the VSTM5-ECD-Ig fusion protein.

FIG. 29 contains data from experiments wherein human CD3 T cells co-cultured with beads coated with various concentration of VSTM5-ECD-Ig fusion protein were analyzed for their level of expression of CD25.

FIG. 30 presents FACS binding results for anti-VSTM5 Fabs reformatted as human IgG1 molecules.

FIGS. 31A and 31B present the DNA and the amino acid sequences, respectively, of the monoclonal antibodies 47-01.D05; 49-01.D06; 49-01.F05; 49-02.C11; 49-01.F01; 50-01.A04; 50-01.B01; 50-01.E02; 50-01.F03; 50-01.D01; 52-01.A07; and 53-01.B11 antibodies disclosed in Examples 12 and 13. The sequences of CDR1, CDR2, CDR3 are underlined. “HC” corresponds to heavy chain; “LC” corresponds to light chain.

FIG. 32 contains the gating scheme used in FACS assays which detected the expression of VSTM5 on leukocytes.

FIG. 33 contains FACS assay results from experiments that detected the expression of VSTM5 on different cell types. As shown by the data therein VSTM5 is highly expressed by monocytes, CD1b1^{low}CD14⁻ cells, and to a lesser degree by eosinophils.

FIG. 34 contains representative results of assays testing the effect of VSTM5-expressing HEK-293T cells on H9 T cells stimulated with anti-human CD3 antibody which demonstrate that this results in reduced activation as manifested by reduced IL-2 secretion in comparison to contacting with control HEK-293T cells transfected with a vector lacking a sequence encoding VSTM5 only (pRp3.1). (Example 11)

FIG. 35 tests the functional effect of VSTM5 binding agents, i.e., anti-VSTM5 specific Abs on T cell activation in the same co-culture assay used in the experiments contained in FIG. 35. In these experiments the assay was performed in the presence of different hIgG1anti-VSTM5 Abs (described in Example 12 and 13 infra).

FIG. 36 contains the results of a co-culture cell based assay testing specific anti-VSTM5 antibodies according to the invention for their ability to modulate the suppressive effect of VSTM5 on T cell activity.

FIG. 37 contains data from experiments wherein human CD3 T cells co-cultured with beads coated with various concentrations of VSTM5-ECD-Ig fusion protein and different anti-VSTM5 Abs according to the invention. The data therein show that three mAbs (50-01.E02, 50-01.A04, 53-01.B11) substantially increased CD25 expression on CD4⁺ T cells, and five other mAbs (49-01.F01, 49-01.D06, 47-01.D05, 49-01.F05, 49-02.C11) did not show an enhancing effect specific to VSTM5 under the same bead assay conditions.

FIG. 38 schematically depicts five different antibody “bins” used to epitopically group anti-VSTM5 antibodies according to the invention.

### DETAILED DESCRIPTION OF THE INVENTION

The present invention, in at least some embodiments, relates to polyclonal and monoclonal antibodies and fragments and/or conjugates thereof, and/or pharmaceutical composition comprising same, and/or diagnostic composition comprising same, wherein these antibodies specifically bind VSTM5 proteins, and preferably modulate (agonize, mimic or antagonize) at least one effect of VSTM5 on immunity, wherein said anti-VSTM5 antibodies are suitable for use as therapeutic and/or diagnostic agents, particularly human treatment and diagnosis, e.g., for treatment and/or diagnosis or aiding in the diagnosis of specific cancers or cancers resistant to existing therapies such as described herein, preferably human, humanized, primatized or chimeric monoclonal antibodies.

As used herein, the term VSTM5 includes any one of the proteins set forth in anyone of SEQ ID NOs: 2, 3, 6, 7, 132, 349, and/or amino acid sequences corresponding to VSTM5 V-set domain set forth in SEQ ID NO: 1, and/or fragments and/or epitopes of the VSTM5 ECD, as set forth in any of SEQ ID NOs: 12-21, and/or variants thereof, such as allelic variants, and/or VSTM5 orthologs and/or fragments thereof, and/or nucleic acid sequences encoding for same. Optionally the term VSTM5 refers to any one of the proteins above that are expressed in cancer, on the cancer cells or in the immune cells infiltrating the tumor, or both and/or stromal cells, prior to or following cancer therapy, optionally prior to or following combination immunotherapy of cancer, as detailed herein.

According to at least some embodiments of the present invention, the antibodies are derived from particular heavy and light chain germline sequences and/or comprise particular structural features such as at least one CDR comprising a particular amino acid sequence, and more typically at least 2, 3, 4, 5 or 6 CDRs of an anti-VSTM5 antibody that has been demonstrated to agonize, mimic or antagonize one or more of VSTM5's effects on immunity. According to at least some embodiments, the present invention provides isolated antibodies, methods of making such antibodies, immunoconjugates and bispecific molecules comprising such antibodies and pharmaceutical and diagnostic compositions containing the antibodies, immunoconjugates, alternative scaffolds or bispecific molecules according to at least some embodiments of the present invention.

According to at least some embodiments the present invention relates to in vitro and in vivo methods of using the antibodies and fragments thereof, to detect any one of VSTM5 proteins.

According to at least some embodiments the present invention further relates to methods of using the foregoing antibodies and fragments and/or conjugates thereof and/or pharmaceutical and/or diagnostic composition comprising same, to treat and/or to diagnose or aid in the diagnosis of cancer, as described herein.

Without wishing to be limited in any way, including by a single hypothesis, and without providing a closed list, it is expected that these anti-VSTM5 antibodies and antigen-binding fragments and conjugates thereof, which have immunostimulatory effects on immune cells will promote anti-cancer or tumor immunity as well as immune reactions against pathogens, infected cells and sepsis alone or in combination with other therapies. Conversely, but also without any limitation, it is expected that anti-VSTM5 antibodies and antigen-binding fragments and conjugates thereof, which have immunoinhibitory effects on immune cells will result in the amelioration of the immune disease, when used alone or in combination with other actives. Particularly, without wishing to be limited in any way, it is expected that anti-VSTM5 antibodies which mimic or enhance the inhibitory effect of VSTM5 on T-cell activation, will result in a dampening of immune responses and amelioration of the immune disease.

Also, anti-VSTM5 antibodies and antigen-binding fragments and conjugates thereof, without wishing to be limited by a single hypothesis, may directly elicit or potentiate cytotoxic activity including antibody dependent or complement dependent cytotoxic activity (ADCC or CDC, respectively) resulting in depletion of VSTM5 expressing cells, including immune cells and/or tumor cells.

Also, it is expected that the subject anti-VSTM5 antibodies which are effective in activating the immune system, without wishing to be limited by a single hypothesis, may be used to attack infectious agents and to reverse diminished immune responses such as those characterized by impaired functionality which can be manifested as T cell exhaustion, reduced cell proliferation and cytokine production, and can be reversed by blocking inhibitory pathways using antibodies as described herein, according to at least some embodiments.

According to at least some embodiments, the present invention provides immunostimulatory antibodies and fragments as described herein, optionally and preferably wherein the antibody binds to human VSTM5 with a K_{D} of 100 nM or less, 50 nM or less, 10 nM or less, or more preferably 1 nM or less (that is, higher binding affinity), or 1 pM or less, wherein K_{D} is determined by known methods, e.g. surface plasmon resonance (SPR), ELISA, KINEXA, and most typically SPR at 25° or 37° C.; and wherein the immunostimulatory antibody preferably exhibits at least one of the following properties: (i) increases immune response, (ii) increases T cell activation, (iii) increases cytotoxic T cell activity, (iv) increases NK cell activity, (v) alleviates T-cell suppression, (vi) increases pro-inflammatory cytokine secretion, (vii) increases IL-2 secretion; (viii) increases interferon-γ production, (ix) increases Th1 response, (x) decrease Th2 response, (xi) decreases or eliminates cell number and/or activity of at least one of regulatory T cells (Tregs), myeloid derived suppressor cells (MDSCs), iMCs, mesenchymal stromal cells, TIE2-expressing monocytes, (xii) reduces regulatory cell activity, and/or the activity of one or more of myeloid derived suppressor cells (MDSCs), iMCs, mesenchymal stromal cells, TIE2-expressing monocytes, (xiii) decreases or eliminates M2 macrophages, (xiv) reduces M2 macrophage pro-tumorigenic activity, (xv) decreases or eliminates N2 neutrophils, (xvi) reduces N2 neutrophils pro-tumorigenic activity, (xvii) reduces inhibition of T cell activation, (xviii) reduces inhibition of CTL activation, (xix) reduces inhibition of NK cell activation, (xx) reverses T cell exhaustion, (xxi) increases T cell response, (xxii) increases activity of cytotoxic cells, (xxiii) stimulates antigen-specific memory responses, (xxiv) elicits apoptosis or lysis of cancer cells, (xxv) stimulates cytotoxic or cytostatic effect on cancer cells, (xxvi) induces direct killing of cancer cells, (xxvii) increases Th17 activity and/or (xxviii) induces complement dependent cytotoxicity and/or antibody dependent cell-mediated cytotoxicity in a mammal, preferably human, with the proviso that said anti-VSTM5 antibody or antigen-binding fragment may elicit an opposite effect to one or more of (i)-(xxviii).

According to other embodiments, the present invention provides immunoinhibitory antibodies and fragments as described herein, optionally and preferably wherein the immunoinhibitory antibody binds to human VSTM5 with a K_{D} of 100 nM or less, 50 nM or less, 10 nM or less, or more preferably 1 nM or less (that is, higher binding affinity), wherein K_{D} is determined by known methods, e.g. surface plasmon resonance (SPR), ELISA, KINEXA, and most typically SPR at 25° or 37° C.; and wherein the immunoinhibitory antibody preferably exhibits at least one of the following properties: (i) decreases immune response, (ii) decreases T cell activation, (iii) decreases cytotoxic T cell activity, (iv) decreases natural killer (NK) cell activity, (v) decreases T-cell activity, (vi) decreases pro-inflammatory cytokine secretion, (vii) decreases IL-2 secretion; (viii) decreases interferon-γ production, (ix) decreases Th1 response, (x) decreases Th2 response, (xi) increases cell number and/or activity of regulatory T cells, (xii) increases regulatory cell activity and/or one or more of myeloid derived suppressor cells (MDSCs), iMCs, mesenchymal stromal cells, TIE2-expressing monocytes, (xiii) increases regulatory cell activity and/or the activity of one or more of myeloid derived suppressor cells (MDSCs), iMCs, mesenchymal stromal cells, TIE2-expressing monocytes, (xiii) increases M2 macrophages, (xiv) increases M2 macrophage activity, (xv) increases N2 neutrophils, (xvi) increases N2 neutrophils activity, (xvii) increases inhibition of T cell activation, (xviii) increases inhibition of CTL activation, (xix) increases inhibition of NK cell activation, (xx) increases T cell exhaustion, (xxi) decreases T cell response, (xxii) decreases activity of cytotoxic cells, (xxiii) reduces antigen-specific memory responses, (xxiv) inhibits apoptosis or lysis of cells, (xxv) decreases cytotoxic or cytostatic effect on cells, (xxvi) reduces direct killing of cells, (xxvii) decreases Th17 activity, and/or (xxviii) reduces complement dependent cytotoxicity and/or antibody dependent cell-mediated cytotoxicity in a mammal, preferably human, with the proviso that said antibody, antigen-binding fragment or conjugate thereof may elicit an opposite effect to one or more of (i)-(xxviii)..

Optionally said immunostimulatory antibody, antibody binding fragment, conjugate, and/or composition containing such is used for treatment of treatment of cancer and/or infectious disease or sepsis; increases immune response against a cancer; reduces activity of regulatory T lymphocytes (T-regs); and/or inhibits iTreg differentiation.

According to at least some embodiments, the present invention provides the foregoing antibodies and fragments thereof, wherein the antibody is a chimeric, humanized, primatized, human antibody, preferably fully human, and/or is an antibody or antibody fragment having CDC or ADCC activities on target cells.

Included in particular are antibodies and fragments that are immune activating or immune suppressing such as antibodies or fragments that target cells via ADCC (antibody dependent cellular cytotoxicity) or CDC (complement dependent cytotoxicity) activities.

According to at least some embodiments, for any of the above described cancers, optionally each of the above described cancer type or subtype may optionally form a separate embodiment and/or may optionally be combined as embodiments or subembodiments.

According to at least some embodiments, for any of the above described cancers, methods of treatment and also uses of the antibodies and pharmaceutical compositions described herein are provided wherein the cancer expresses VSTM5 polypeptides comprised in SEQ ID NOs: 6, 7, 132, 349 and/or their corresponding extracellular domains, selected from the group consisting of any one of SEQ ID NOs: 2, 3, and/or fragments, such as for example any of SEQ ID NOs:1, 12-21, and/or epitopes thereof, on the cancer cells and/or on the immune cells infiltrating the tumor, and/or stromal cells, wherein the VSTM5 expression is either prior to or following cancer therapy, optionally prior to or following combination immunotherapy of cancer.

Optionally, said cancer, said immune infiltrate or both, and/or stromal cells express VSTM5 at a sufficient level and said cancer is as described herein, wherein VSTM5 expression on any of the cells listed above could be either present prior to cancer treatment or induced post treatment. By immune infiltrate it is meant immune cells infiltrating to the tumor or to the area of the cancerous cells. By “expressing VSTM5 at a sufficient level” it is meant that such cells express VSTM5 protein at a high enough level according to an assay. For example, if the assay is IHC (immunohistochemistry), and expression is measured on a scale of 0 to 3 (0—no expression, 1—faint staining, 2—moderate and 3—strong expression), then a sufficient level of VSTM5 expression would optionally be at least 1, preferably be at least 2 and more preferably be at least 3. Optionally the antibodies or immune molecules as described herein may be used for such an assay. Also, in some instances a “sufficient level” detected by the assay may refer to a level of VSTM5 expression such that administration of an anti-VSTM5 antibody or antigen-binding fragment according to the invention is likely to elicit a significant therapeutic benefit in a subject with a disease condition characterized by cells exhibiting such level of VSTM5 expression.

Standard assays to evaluate the binding ability of the antibodies toward VSTM5 are known in the art, including for example, ELISAs, Western blots and RIAs. Suitable assays are described in detail in the Examples. The binding kinetics (e.g., binding affinity) of the antibodies also can be assessed by standard assays known in the art, such as by surface plasmon resonance analysis, ELISA and KINEXA.

According to at least some embodiments, the subject anti-VSTM5 immune molecule, antibody, antibody binding fragment, and/or composition containing is used for treatment of immune related diseases and/or for reducing the undesirable immune activation that follows gene therapy.

As disclosed herein, according to at least some embodiments, the invention embraces anti-VSTM5 antibodies and fragments, and variants thereof, e.g., wherein the VH and VL sequences of different anti-VSTM5 antibodies can be “mixed and matched” to create other anti-VSTM5, binding molecules according to at least some embodiments of the invention. VSTM5 binding of such “mixed and matched” antibodies can be tested using the binding assays described above. e.g., ELISAs). Preferably, when VH and VL chains are mixed and matched, a VH sequence from a particular VH/VL pairing is replaced with a structurally similar VH sequence. Likewise, preferably a VL sequence from a particular VH/VL pairing is replaced with a structurally similar VL sequence. For example, the VH and VL sequences of homologous antibodies are particularly amenable for mixing and matching.

Optionally, the antibody comprises CDR amino acid sequences selected from the group consisting of (a) sequences as listed herein; (b) sequences that differ from those CDR amino acid sequences specified in (a) by 1, 2, 3, 4, 5, 6, 7, 8, 9, 10 or more conservative amino acid substitutions except for the Serine residue in heavy chain CDR3 at position 100A (Kabat numbering system); (c) amino acid sequences having 90% or greater, 95% or greater, 98% or greater, or 99% or greater sequence identity to the sequences specified in (a) or (b); (d) a polypeptide having an amino acid sequence encoded by a polynucleotide having a nucleic acid sequence encoding the amino acids as listed herein.

Optionally, for any antibody or fragment described herein, the antibody may be bispecific, meaning that one arm of the Ig molecule is specific for binding to the target protein or epitope as described herein, and the other arm of the Ig molecule has a different specificity that can enhance or redirect the biological activity of the antibody or fragment. In this regard, a multi-specific antibody is also considered to be at least bispecific. The antibody or fragment also can be multi-specific in the sense of being multi-valent.

According to at least some embodiments the invention relates to protein scaffolds with specificities and affinities in a range similar to specific antibodies. According to at least some embodiments the present invention relates to an antigen-binding construct comprising a protein scaffold which is linked to one or more epitope-binding domains. Such engineered protein scaffolds are usually obtained by designing a random library with mutagenesis focused at a loop region or at an otherwise permissible surface area and by selection of variants against a given target via phage display or related techniques. According to at least some embodiments the invention relates to alternative scaffolds including, but not limited to, anticalins, DARPins, Armadillo repeat proteins, protein A, lipocalins, fibronectin domain, ankyrin consensus repeat domain, thioredoxin, chemically constrained peptides and the like. According to at least some embodiments the invention relates to alternative scaffolds that are used as therapeutic agents for treatment of cancer as recited herein, as well as for in vivo diagnostics.

In order that the present invention in various embodiments may be more readily understood, certain terms are first defined. Additional definitions are set forth throughout the specification in particular in the detailed description.

### DEFINITIONS

Unless defined otherwise, all technical and scientific terms used herein have the same meaning as those commonly understood by one of ordinary skill in the art to which this invention belongs. Although methods and materials similar or equivalent to those described herein may be used in the invention or testing of the present invention, suitable methods and materials are described herein. The materials, methods and examples are illustrative only, and are not intended to be limiting. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques may be used for chemical syntheses, chemical analyses, pharmaceutical preparation, formulation, and delivery, and treatment of patients.

As used in the description herein and throughout the claims that follow, the meaning of “a,” “an,” and “the” includes plural reference unless the context clearly dictates otherwise.

“Activating receptor,” as used herein, refers broadly to immune cell receptors that bind antigen, complexed antigen (e.g., in the context of MHC molecules), Ig-fusion proteins, ligands, or antibodies. Activating receptors but are not limited to T cell receptors (TCRs), B cell receptors (BCRs), cytokine receptors, LPS receptors, complement receptors, and Fc receptors. For example, T cell receptors are present on T cells and are associated with CD3 molecules. T cell receptors are stimulated by antigen in the context of MHC molecules (as well as by polyclonal T cell activating reagents). T cell activation via the TCR results in numerous changes, e.g., protein phosphorylation, membrane lipid changes, ion fluxes, cyclic nucleotide alterations, RNA transcription changes, protein synthesis changes, and cell volume changes. For example, T cell receptors are present on T cells and are associated with CD3 molecules. T cell receptors are stimulated by antigen in the context of MHC molecules (as well as by polyclonal T cell activating reagents). T cell activation via the TCR results in numerous changes, e.g., protein phosphorylation, membrane lipid changes, ion fluxes, cyclic nucleotide alterations, RNA transcription changes, protein synthesis changes, and cell volume changes.

“Adjuvant” as used herein, refers to an agent used to stimulate the immune system and increase the response to a vaccine, without having any specific antigenic effect in itself.

“Aids in the diagnosis” or “aids in the detection” of a disease herein means that the expression level of a particular marker polypeptide or expressed RNA is detected alone or in association with other markers in order to assess whether a subject has cells characteristic of a particular disease condition or the onset of a particular disease condition or comprises immune disfunction such as immunosuppression characterized by VSTM5 expression or abnormal immune upregulation characterized by cells having reduced VSTM5 levels, such as during autoimmunity.

“Allergic disease,” as used herein, refers broadly to a disease involving allergic reactions. More specifically, an “allergic disease” is defined as a disease for which an allergen is identified, where there is a strong correlation between exposure to that allergen and the onset of pathological change, and where that pathological change has been proven to have an immunological mechanism. Herein, an immunological mechanism means that leukocytes show an immune response to allergen stimulation.

“Amino acid,” as used herein refers broadly to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified (e.g., hydroxyproline, γ-carboxyglutamate, and O-phosphoserine.) Amino acid analogs refers to compounds that have the same basic chemical structure as a naturally occurring amino acid (i.e., a carbon that is bound to a hydrogen, a carboxyl group, an amino group), and an R group (e.g., homoserine, norleucine, methionine sulfoxide, methionine methyl sulfonium.) Analogs may have modified R groups (e.g., norleucine) or modified peptide backbones, but retain the same basic chemical structure as a naturally occurring amino acid Amino acid mimetics refers to chemical compounds that have a structure that is different from the general chemical structure of an amino acid, but that functions in a manner similar to a naturally occurring amino acid.

“Anergy” or “tolerance,” or “prolonged antigen-specific T cell suppression” as used herein refers broadly to refractivity to activating receptor-mediated stimulation. Refractivity is generally antigen-specific and persists after exposure to the tolerizing antigen has ceased. For example, anergy in T cells (as opposed to unresponsiveness) is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory molecule) results in failure to produce cytokines and, thus, failure to proliferate. Anergic T cells can, however, mount responses to unrelated antigens and can proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer (Kang et al. (1992) Science 257:1134). Modulation of a costimulatory signal results in modulation of effector function of an immune cell.

“Antibody”, as used herein, refers broadly to an “antigen-binding portion” of an antibody (also used interchangeably with “antibody portion,” “antigen-binding fragment,” “antibody fragment”), as well as whole antibody molecules. The term “antigen-binding portion”, as used herein, refers to one or more fragments of an antibody that retain the ability to specifically bind to an antigen (e.g., VSTM5 or specific portions thereof)). The term “antibody” as referred to herein includes whole polyclonal and monoclonal antibodies and any antigen-binding fragment (i.e., “antigen-binding portion”) or single chains thereof. An “antibody” refers to a glycoprotein comprising at least two heavy (H) chains and two light (L) chains inter-connected by disulfide bonds, or an antigen-binding portion thereof. Each heavy chain is comprised of at least one heavy chain variable region (abbreviated herein as V_{H}) and a heavy chain constant region. The heavy chain constant region is comprised of three domains, C_{H1}, C_{H2} and C_{H3}. Each light chain is comprised of at least one light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

More generally, the term “antibody” is intended to include any polypeptide chain-containing molecular structure with a specific shape that fits to and recognizes an epitope, where one or more non-covalent binding interactions stabilize the complex between the molecular structure and the epitope. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammals, chicken, other avians, etc., are considered to be “antibodies.” A preferred source for producing antibodies useful as starting material according to the invention is rabbits. Numerous antibody coding sequences have been described; and others may be raised by methods well-known in the art. Examples thereof include chimeric antibodies, human antibodies and other non-human mammalian antibodies, humanized antibodies, single chain antibodies (such as scFvs), camelbodies, nanobodies, IgNAR (single-chain antibodies derived from sharks), small-modular immunopharmaceuticals (SMIPs), and antibody fragments such as Fabs, Fab′, F(ab′)2 and the like. See Streltsov V A, et al., “Structure of a shark IgNAR antibody variable domain and modeling of an early-developmental isotype”, Protein Sci. 2005 November; 14(11):2901-9. Epub 2005 Sep. 30; Greenberg A S, et al., “A new antigen receptor gene family that undergoes rearrangement and extensive somatic diversification in sharks”, Nature. 1995 Mar. 9; 374(6518):168-73; Nuttall S D, et al., “Isolation of the new antigen receptor from wobbegong sharks, and use as a scaffold for the display of protein loop libraries”, Mol. Immunol. 2001 August; 38(4):313-26; Hamers-Casterman C, et al., “Naturally occurring antibodies devoid of light chains”, Nature, 1993 Jun. 3; 363(6428):446-8; Gill D S, et al., “Biopharmaceutical drug discovery using novel protein scaffolds”, Curr Opin Biotechnol. 2006 December; 17(6):653-8. Epub 2006 Oct. 19. Antibodies or antigen-binding fragments may e.g., be produced by genetic engineering. In this technique, as with other methods, antibody-producing cells are sensitized to the desired antigen or immunogen. The messenger RNA isolated from antibody producing cells is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library is constructed by combining the heavy chain gene library with the light chain gene library. This results in a library of clones which co-express a heavy and light chain (resembling the Fab fragment or antigen-binding fragment of an antibody molecule). The vectors that carry these genes are co-transfected into a host cell. When antibody gene synthesis is induced in the transfected host, the heavy and light chain proteins self-assemble to produce active antibodies that can be detected by screening with the antigen or immunogen.

Antibody coding sequences of interest include those encoded by native sequences, as well as nucleic acids that, by virtue of the degeneracy of the genetic code, are not identical in sequence to the disclosed nucleic acids, and variants thereof. Variant polypeptides can include amino acid (aa) substitutions, additions or deletions. The amino acid substitutions can be conservative amino acid substitutions or substitutions to eliminate non-essential amino acids, such as to alter a glycosylation site, or to minimize misfolding by substitution or deletion of one or more cysteine residues that are not necessary for function. Variants can be designed so as to retain or have enhanced biological activity of a particular region of the protein (e.g., a functional domain, catalytic amino acid residues, etc). Variants also include fragments of the polypeptides disclosed herein, particularly biologically active fragments and/or fragments corresponding to functional domains. Techniques for in vitro mutagenesis of cloned genes are known. Also included in the subject invention are polypeptides that have been modified using ordinary molecular biological techniques so as to improve their resistance to proteolytic degradation or to optimize solubility properties or to render them more suitable as a therapeutic agent. Chimeric antibodies according to the invention include those made by recombinant means by combining the variable light and heavy chain regions (V_{L} and V_{H}), obtained from antibody producing cells of one species with the constant light and heavy chain regions from another. Typically chimeric antibodies utilize rodent or rabbit variable regions and human constant regions, in order to produce an antibody with predominantly human domains. The production of such chimeric antibodies is well known in the art, and may be achieved by standard means (as described, e.g., in U.S. Pat. No. 5,624,659, incorporated herein by reference in its entirety). It is further contemplated that the human constant regions of chimeric antibodies of the invention may be selected from IgG1, IgG2, IgG3, IgG4, constant regions. Antibodies herein include humanized antibodies as defined infra. Also, “antibodies” includes as well as entire immunoglobulins (or their recombinant counterparts), immunoglobulin fragments comprising the epitope binding site (e.g., Fab′, F(ab′)2, or other antigen-binding fragments, including further minimal immunoglobulins which may be designed utilizing recombinant immunoglobulin techniques and “Fv” immunoglobulins reduced by synthesizing a fused variable light chain region and a variable heavy chain region. Combinations of antibodies are also of interest, e.g. diabodies, which comprise two distinct Fv specificities Also antibodies according to the invention is intended to include, SMIPs (small molecule immunopharmaceuticals), camelbodies, nanobodies, and IgNAR are encompassed by immunoglobulin fragments. Further, “antibodies” herein includes immunoglobulins and fragments thereof which may be modified post-translationally, e.g. to add effector moieties such as chemical linkers, detectable moieties, such as fluorescent dyes, enzymes, toxins, substrates, bioluminescent materials, radioactive materials, chemiluminescent moieties and the like, or specific binding moieties, such as streptavidin, avidin, or biotin, and the like may be utilized in the methods and compositions of the present invention. Examples of additional effector molecules are provided infra.

The antigen-binding function of an antibody can be performed by fragments of a full-length antibody. Non-limiting examples of antigen-binding fragments encompassed within the term “antigen-binding portion” of an antibody include (a) a Fab fragment, a monovalent fragment consisting of the V_{L}, V_{H}, C_{L} and C_{H1} domains; (b) a F(ab′)2 fragment, a bivalent fragment comprising two Fab fragments linked by a disulfide bridge at the hinge region; (c) a F_{d} fragment consisting of the V_{H} and C_{H1} domains; (d) a Fᵥ fragment consisting of the V_{L} and V_{H} domains of a single arm of an antibody; (e) a dAb fragment (Ward, et al. (1989) Nature 341: 544-546), which consists of a V_{H} domain; and (f) an isolated complementarily determining region (CDR). Furthermore, although the two domains of the Fᵥ fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv). See e.g., Bird, et al. (1988) Science 242: 423-426; Huston, et al. (1988) Proc Natl. Acad. Sci. USA 85: 5879-5883; and Osbourn, et al. (1998) Nat. Biotechnol. 16: 778. Single chain antibodies are also intended to be encompassed within the term “antigen-binding portion” of an antibody. Any V_{H} and V_{L} sequences of specific scFv can be linked to human immunoglobulin constant region cDNA or genomic sequences, in order to generate expression vectors encoding complete IgG molecules or other isotypes. V_{H} and V_{L} can also be used in the generation of Fab, Fv, or other fragments of immunoglobulins using either protein chemistry or recombinant DNA technology. Other forms of single chain antibodies, such as diabodies are also encompassed. Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen-binding sites. See e.g., Holliger, et al. (1993) Proc Natl. Acad. Sci. USA 90: 6444-6448; Poljak, et al. (1994) Structure 2: 1121-1123.

Still further, an antibody or antigen-binding portion thereof (antigen-binding fragment, antibody fragment, antibody portion) may be part of a larger immunoadhesion molecules, formed by covalent or noncovalent association of the antibody or antibody portion with one or more other proteins or peptides. Examples of immunoadhesion molecules include use of the streptavidin core region to make a tetrameric scFv molecule (Kipriyanov, et al. (1995) Hum. Antibodies Hybridomas 6: 93-101) and use of a cysteine residue, a marker peptide and a C-terminal polyhistidine tag to make bivalent and biotinylated scFv molecules. Kipriyanov, et al. (1994) Mol. Immunol. 31: 1047-1058. Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

Antibodies may be polyclonal, monoclonal, xenogeneic, allogeneic, syngeneic, or modified forms thereof, e.g., humanized, chimeric. Preferably, antibodies of the invention bind specifically or substantially specifically to VSTM5 molecules. The terms “monoclonal antibodies” and “monoclonal antibody composition”, as used herein, refer to a population of antibody molecules that contain only one species of an antigen-binding site capable of immunoreacting with a particular epitope of an antigen, whereas the term “polyclonal antibodies” and “polyclonal antibody composition” refer to a population of antibody molecules that contain multiple species of antigen-binding sites capable of interacting with a particular antigen. A monoclonal antibody composition, typically displays a single binding affinity for a particular antigen with which it immunoreacts. A “desired antibody” herein refers generally to a parent antibody specific to a target, i.e., or a chimeric or humanized antibody or a binding portion thereof derived therefrom as described herein.

“Antibody recognizing an antigen” and “an antibody specific for an antigen” are used interchangeably herein with the term “an antibody which binds specifically to an antigen.”

“Antibody that specifically binds to human VSTM5 proteins” is intended to refer to an antibody that binds to VSTM5 proteins, preferably one with a K_{D} of 10⁻⁷ M, more preferably 5×10⁻⁸ M or more preferably 3×10⁻⁸ M or less, 10⁻⁸ M, even more preferably 1×10⁻⁹ M or less, even more preferably 1×10⁻¹⁰ M, even more preferably 1×10⁻¹¹ M and even more preferably 1×10⁻¹² M or less.

“Antigen,” as used herein, refers broadly to a molecule or a portion of a molecule capable of being bound by an antibody which is additionally capable of inducing an animal to produce an antibody capable of binding to an epitope of that antigen. An antigen may have one epitope, or have more than one epitope. The specific reaction referred to herein indicates that the antigen will react, in a highly selective manner, with its corresponding antibody and not with the multitude of other antibodies which may be evoked by other antigens. In the case of a desired enhanced immune response to particular antigens of interest, antigens include, but are not limited to infectious disease antigens for which a protective immune response may be elicited are exemplary.

“Antigen-binding portion” of an antibody (or simply “antibody portion”), as used herein, refers to one or more fragments of an antibody that retain the ability to specifically bind to an antigen (e.g., VSTM5 molecules, and/or a fragment thereof). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the Variable Light (V_{L}), Variable Heavy (V_{H}), Constant light (C_{L}) and C_{H1} domains; (ii) a F(ab′)2 fragment, a bivalent fragment comprising two Fab fragments linked by a disulfide bridge at the hinge region; (iii) a F_{d} fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

“Antigen presenting cell,” as used herein, refers broadly to professional antigen presenting cells (e.g., B lymphocytes, monocytes, dendritic cells, and Langerhans cells) as well as other antigen presenting cells (e.g., keratinocytes, endothelial cells, astrocytes, fibroblasts, and oligodendrocytes).

“Antisense nucleic acid molecule,” as used herein, refers broadly to a nucleotide sequence which is complementary to a “sense” nucleic acid encoding a protein (e.g., complementary to the coding strand of a double-stranded cDNA molecule) complementary to an mRNA sequence or complementary to the coding strand of a gene. Accordingly, an antisense nucleic acid molecule can hydrogen bond to a sense nucleic acid molecule.

“Apoptosis”, as used herein, refers broadly to programmed cell death which can be characterized using techniques which are known in the art. Apoptotic cell death can be characterized by cell shrinkage, membrane blebbing, and chromatin condensation culminating in cell fragmentation. Cells undergoing apoptosis also display a characteristic pattern of internucleosomal DNA cleavage.

“Asthma,” as used herein, refers broadly to an allergic disorder of the respiratory system characterized by inflammation, narrowing of the airways and increased reactivity of the airways to inhaled agents. Asthma is frequently, although not exclusively, associated with atopic or allergic symptoms.

“Autoimmunity” or “autoimmune disease or condition,” as used herein, refers broadly to a disease or disorder arising from and directed against an individual's own tissues or a co-segregate or manifestation thereof or resulting condition therefrom. Herein autoimmune conditions include inflammatory or allergic conditions characterized by a host immune reaction against self-antigens, such as rheumatoid arthritis and numerous others.

“B cell receptor” (BCR),” as used herein, refers broadly to the complex between membrane Ig (mIg) and other transmembrane polypeptides (e.g., Ig α and Igβ) found on B cells. The signal transduction function of mIg is triggered by crosslinking of receptor molecules by oligomeric or multimeric antigens. B cells can also be activated by anti-immunoglobulin antibodies. Upon BCR activation, numerous changes occur in B cells, including tyrosine phosphorylation.

“Cancer,” as used herein, refers broadly to any neoplastic disease (whether invasive or metastatic) characterized by abnormal and uncontrolled cell division causing malignant growth or tumor (e.g., unregulated cell growth.) The term “cancer” or “cancerous” as used herein should be understood to encompass any neoplastic disease (whether invasive, non-invasive or metastatic) which is characterized by abnormal and uncontrolled cell division causing malignant growth or tumor, non-limiting examples of which are described herein. This includes any physiological condition in mammals that is typically characterized by unregulated cell growth. Examples of cancer are exemplified in the working examples and also are described within the specification.

“Cancer therapy” herein refers to any method which prevents or treats cancer or ameliorates one or more of the symptoms of cancer. Typically such therapies will comprises administration of an immunostimulatory anti-VSTM5 antibody or antigen-binding fragment, conjugate or composition containing according to the invention either alone or more typically in combination with chemotherapy or radiotherapy or other biologics and for enhancing the activity thereof, i.e., in individuals wherein VSTM5 expression suppress antitumor responses and the efficacy of chemotherapy or radiotherapy or biologic efficacy. Any chemotherapeutic agent exhibiting anticancer activity can be used according to the present invention; various non-limiting examples are described in the specification.

“Chimeric antibody,” as used herein, refers broadly to an antibody molecule in which the constant region, or a portion thereof, is altered, replaced or exchanged so that the antigen-binding site (variable region) is linked to a constant region of a different or altered class, effector function and/or species, or an entirely different molecule which confers new properties to the chimeric antibody, e.g., an enzyme, toxin, hormone, growth factor, drug, the variable region or a portion thereof, is altered, replaced or exchanged with a variable region having a different or altered antigen specificity.

“Coding region,” as used herein, refers broadly to regions of a nucleotide sequence comprising codons which are translated into amino acid residues, whereas the term “noncoding region” refers to regions of a nucleotide sequence that are not translated into amino acids (e.g., 5′ and 3′ untranslated regions).

“Conservatively modified variants,” as used herein, applies to both amino acid and nucleic acid sequences, and with respect to particular nucleic acid sequences, refers broadly to conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. “Silent variations” are one species of conservatively modified nucleic acid variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) may be modified to yield a functionally identical molecule.

“Complementarity determining region,” “hypervariable region,” or “CDR,” as used herein, refers broadly to one or more of the hyper-variable or complementarily determining regions (CDRs) found in the variable regions of light or heavy chains of an antibody. See Kabat, et al. (1987) “Sequences of Proteins of Immunological Interest” National Institutes of Health, Bethesda, Md. These expressions include the hypervariable regions as defined by Kabat, et al. (1983) “Sequences of Proteins of Immunological Interest” U.S. Dept. of Health and Human Services or the hypervariable loops in 3-dimensional structures of antibodies. Chothia and Lesk (1987) J. Mol. Biol. 196: 901-917. The CDRs in each chain are held in close proximity by framework regions and, with the CDRs from the other chain, contribute to the formation of the antigen-binding site. Within the CDRs there are select amino acids that have been described as the selectivity determining regions (SDRs) which represent the critical contact residues used by the CDR in the antibody-antigen interaction. Kashmiri (2005) Methods 36: 25-34.

“Control amount,” as used herein, refers broadly to a marker can be any amount or a range of amounts to be compared against a test amount of a marker. For example, a control amount of a marker may be the amount of a marker in a patient with a particular disease or condition or a person without such a disease or condition. A control amount can be either in absolute amount (e.g., microgram/ml) or a relative amount (e.g., relative intensity of signals).

“Costimulatory receptor,” as used herein, refers broadly to receptors which transmit a costimulatory signal to an immune cell, e.g., CD28 or ICOS. As used herein, the term “inhibitory receptors” includes receptors which transmit a negative signal to an immune cell, e.g., a T cell or an NK cell.

“Costimulate,” as used herein, refers broadly to the ability of a costimulatory molecule to provide a second, non-activating, receptor-mediated signal (a “costimulatory signal”) that induces proliferation or effector function. For example, a costimulatory signal can result in cytokine secretion (e.g., in a T cell that has received a T cell-receptor-mediated signal) Immune cells that have received a cell receptor-mediated signal (e.g., via an activating receptor) may be referred to herein as “activated immune cells.” With respect to T cells, transmission of a costimulatory signal to a T cell involves a signaling pathway that is not inhibited by cyclosporin A. In addition, a costimulatory signal can induce cytokine secretion (e.g., IL-2 and/or IL-10) in a T cell and/or can prevent the induction of unresponsiveness to antigen, the induction of anergy, or the induction of cell death in the T cell.

“Costimulatory polypeptide” or “costimulatory molecule” herein refers to a polypeptide that, upon interaction with a cell-surface molecule on T cells, modulates T cell responses.

“Costimulatory signaling” as used herein is the signaling activity resulting from the interaction between costimulatory polypeptides on antigen presenting cells and their receptors on T cells during antigen-specific T cell responses. Without wishing to be limited by a single hypothesis, the antigen-specific T cell response is believed to be mediated by two signals: 1) engagement of the T cell Receptor (TCR) with antigenic peptide presented in the context of MHC (signal 1), and 2) a second antigen-independent signal delivered by contact between different costimulatory receptor/ligand pairs (signal 2). Without wishing to be limited by a single hypothesis, this “second signal” is critical in determining the type of T cell response (activation vs inhibition) as well as the strength and duration of that response, and is regulated by both positive and negative signals from costimulatory molecules, such as the B7 family of proteins.

“B7” polypeptide herein means a member of the B7 family of proteins that costimulate T cells including, but not limited to B7-1, B7-2, B7-DC, B7-H5, B7-H1, B7-H2, B7-H3, B7-H4, B7-H6, B7-S3 and biologically active fragments and/or variants thereof. Representative biologically active fragments include the extracellular domain or fragments of the extracellular domain that costimulate T cells.

“Cytoplasmic domain,” as used herein, refers broadly to the portion of a protein which extends into the cytoplasm of a cell.

“Diagnostic,” as used herein, refers broadly to identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

“Diagnosing,” or “aiding in the diagnosis” as used herein refers broadly to classifying a disease or a symptom, and/or determining the likelihood that an individual has a disease condition (e.g., based on absence or presence of VSTM5 expression, and/or increased or decreased expression by immune, stromal and/or putative diseased cells); determining a severity of the disease, monitoring disease progression, forecasting an outcome of a disease and/or prospects of recovery. The term “detecting” may also optionally encompass any of the foregoing. Diagnosis of a disease according to the present invention may, in some embodiments, be affected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject.

“Effective amount,” as used herein, refers broadly to the amount of a compound, antibody, antigen, or cells that, when administered to a patient for treating a disease, is sufficient to effect such treatment for the disease. The effective amount may be an amount effective for prophylaxis, and/or an amount effective for prevention. The effective amount may be an amount effective to reduce, an amount effective to prevent the incidence of signs/symptoms, to reduce the severity of the incidence of signs/symptoms, to eliminate the incidence of signs/symptoms, to slow the development of the incidence of signs/symptoms, to prevent the development of the incidence of signs/symptoms, and/or effect prophylaxis of the incidence of signs/symptoms. The “effective amount” may vary depending on the disease and its severity and the age, weight, medical history, susceptibility, and pre-existing conditions, of the patient to be treated. The term “effective amount” is synonymous with “therapeutically effective amount” for purposes of this invention.

“Extracellular domain,” or “ECD” as used herein refers broadly to the portion of a protein that extend from the surface of a cell.

“Expression vector,” as used herein, refers broadly to any recombinant expression system for the purpose of expressing a nucleic acid sequence of the invention in vitro or in vivo, constitutively or inducibly, in any cell, including prokaryotic, yeast, fungal, plant, insect or mammalian cell. The term includes linear or circular expression systems. The term includes expression systems that remain episomal or integrate into the host cell genome. The expression systems can have the ability to self-replicate or not, i.e., drive only transient expression in a cell. The term includes recombinant expression cassettes which contain only the minimum elements needed for transcription of the recombinant nucleic acid.

“Family,” as used herein, refers broadly to the polypeptide and nucleic acid molecules of the invention is intended to mean two or more polypeptide or nucleic acid molecules having a common structural domain or motif and having sufficient amino acid or nucleotide sequence homology as defined herein. Family members can be naturally or non-naturally occurring and can be from either the same or different species. For example, a family can contain a first polypeptide of human origin, as well as other, distinct polypeptides of human origin or alternatively, can contain homologues of non-human origin (e.g., monkey polypeptides.) Members of a family may also have common functional characteristics.

“Fc receptor” (FcRs) as used herein, refers broadly to cell surface receptors for the Fc portion of immunoglobulin molecules (Igs). Fc receptors are found on many cells which participate in immune responses. Among the human FcRs that have been identified so far are those which recognize IgG (designated FcγR), IgE (FcεR1), IgA (FcαR), and polymerized IgM/A (FcεμR). FcRs are found in the following cell types: FcεRI (mast cells), FcεRII (many leukocytes), FcαR (neutrophils), and FcμαR (glandular epithelium, hepatocytes). Hogg (1988) Immunol. Today 9: 185-86. The widely studied FcγRs are central in cellular immune defenses, and are responsible for stimulating the release of mediators of inflammation and hydrolytic enzymes involved in the pathogenesis of autoimmune disease. Unkeless (1988) Annu. Rev. Immunol. 6: 251-87. The FcγRs provide a crucial link between effector cells and the lymphocytes that secrete Ig, since the macrophage/monocyte, polymorphonuclear leukocyte, and natural killer (NK) cell FcγRs confer an element of specific recognition mediated by IgG. Human leukocytes have at least three different receptors for IgG: hFcpRI (found on monocytes/macrophages), hFcγRII (on monocytes, neutrophils, eosinophils, platelets, possibly B cells, and the K562 cell line), and FcγIII (on NK cells, neutrophils, eosinophils, and macrophages).

“Framework region” or “FR,” as used herein refers broadly to one or more of the framework regions within the variable regions of the light and heavy chains of an antibody. See Kabat, et al. (1987) “Sequences of Proteins of Immunological Interest” National Institutes of Health, Bethesda, Md. These expressions include those amino acid sequence regions interposed between the CDRs within the variable regions of the light and heavy chains of an antibody.

“Heterologous,” as used herein, refers broadly to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid (e.g., a promoter from one source and a coding region from another source.) Similarly, a heterologous protein indicates that the protein comprises two or more subsequences that are not found in the same relationship to each other in nature (e.g., a fusion protein).

“High affinity,” as used herein, refers broadly to an antibody having a K_{D} of at least 10⁻⁷ M, more preferably at least 10⁻⁸ M and even more preferably at least 10⁻⁹ or 10-¹⁰ M for a target antigen.

“High affinity” for an IgG antibody herein refers to an antibody having a K_{D} of 10⁻⁶ M or less, 10⁻⁷ M or less, preferably 10⁻⁸ M or less, more preferably 10⁻⁹ M or less and even more preferably 10⁻¹⁰ M or less for a target antigen. However, “high affinity” binding can vary for other antibody isotypes. For example, “high affinity” binding for an IgM isotype refers to an antibody having a K_{D} of 10⁻⁷M or less, more preferably 10⁻⁸ M or less.

“Homology,” as used herein, refers broadly to a degree of similarity between a nucleic acid sequence and a reference nucleic acid sequence or between a polypeptide sequence and a reference polypeptide sequence. Homology may be partial or complete. Complete homology indicates that the nucleic acid or amino acid sequences are identical. A partially homologous nucleic acid or amino acid sequence is one that is not identical to the reference nucleic acid or amino acid sequence. The degree of homology can be determined by sequence comparison, for example using BlastP software of the National Center of Biotechnology Information (NCBI) using default parameters. The term “sequence identity” may be used interchangeably with “homology.”

“Host cell,” as used herein, refers broadly to refer to a cell into which a nucleic acid molecule of the invention, such as a recombinant expression vector of the invention, has been introduced. Host cells may be prokaryotic cells (e.g., E. coli), or eukaryotic cells such as yeast, insect (e.g., SF9), amphibian, or mammalian cells such as CHO, HeLa, HEK-293, e.g., cultured cells, explants, and cells in vivo. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, progeny may not, in fact, be identical to the parent cell, but are still included within the scope of the term as used herein.

“Human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell. This includes fully human monoclonal antibodies and conjugates and variants thereof, e.g., which are bound to effector agents such as therapeutics or diagnostic agents.

“Humanized antibody,” as used herein, refers broadly to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

“Hybridization,” as used herein, refers broadly to the physical interaction of complementary (including partially complementary) polynucleotide strands by the formation of hydrogen bonds between complementary nucleotides when the strands are arranged antiparallel to each other.

“IgV domain” and “IgC domain” as used herein, refer broadly to Ig superfamily member domains. These domains correspond to structural units that have distinct folding patterns called Ig folds. Ig folds are comprised of a sandwich of two 13 sheets, each consisting of antiparallel 13 strands of 5-10 amino acids with a conserved disulfide bond between the two sheets in most, but not all, domains. IgC domains of Ig, TCR, and MHC molecules share the same types of sequence patterns and are called the C1 set within the Ig superfamily. Other IgC domains fall within other sets. IgV domains also share sequence patterns and are called V set domains. IgV domains are longer than C-domains and form an additional pair of 13 strands.

“Immune cell,” as used herein, refers broadly to cells that are of hematopoietic origin and that play a role in the immune response Immune cells include but are not limited to lymphocytes, such as B cells and T cells; natural killer cells; dendritic cells, and myeloid cells, such as monocytes, macrophages, eosinophils, mast cells, basophils, and granulocytes.

“Immunoassay,” as used herein, refers broadly to an assay that uses an antibody to specifically bind an antigen. The immunoassay may be characterized by the use of specific binding properties of a particular antibody to isolate, target, and/or quantify the antigen.

“Immune related disease (or disorder or condition)” as used herein should be understood to encompass any disease disorder or condition selected from the group including but not limited to autoimmune diseases, inflammatory disorders and immune disorders associated with graft transplantation rejection, such as acute and chronic rejection of organ transplantation, allogenic stem cell transplantation, autologous stem cell transplantation, bone marrow transplantation, and graft versus host disease.

“Immune response,” as used herein, refers broadly to T cell-mediated and/or B cell-mediated immune responses that are influenced by modulation of T cell costimulation. Exemplary immune responses include B cell responses (e.g., antibody production) T cell responses (e.g., cytokine production, and cellular cytotoxicity) and activation of cytokine responsive cells, e.g., macrophages. As used herein, the term “downmodulation” with reference to the immune response includes a diminution in any one or more immune responses, while the term “upmodulation” with reference to the immune response includes an increase in any one or more immune responses. It will be understood that upmodulation of one type of immune response may lead to a corresponding downmodulation in another type of immune response. For example, upmodulation of the production of certain cytokines (e.g., IL-10) can lead to downmodulation of cellular immune responses.

“Immunologic”, “immunological” or “immune” response herein refer to the development of a humoral (antibody mediated) and/or a cellular (mediated by antigen-specific T cells or their secretion products) response directed against a peptide in a recipient patient. Such a response can be an active response induced by administration of immunogen or a passive response induced by administration of antibody or primed T-cells. Without wishing to be limited by a single hypothesis, a cellular immune response is elicited by the presentation of polypeptide epitopes in association with Class II or Class I MHC molecules to activate antigen-specific CD4⁺ T helper cells and/or CD8⁺ cytotoxic T cells, respectively. The response may also involve activation of monocytes, macrophages, NK cells, basophils, dendritic cells, astrocytes, microglia cells, eosinophils, activation or recruitment of neutrophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject.

“Immunogenic agent” or “immunogen” is a moiety capable of inducing an immunological response against itself on administration to a mammal, optionally in conjunction with an adjuvant.

“Infectious agent” herein refers to any pathogen or agent that infects mammalian cells, preferably human cells and causes a disease condition. Examples thereof include bacteria, yeast, fungi, protozoans, mycoplasma, viruses, prions, and parasites and which are described in this specification.

“Infectious agent antigen” herein means a compound, e.g., peptide, polypeptide, glycopeptide, glycoprotein, and the like, or a conjugate, fragment or variant thereof, which compound is expressed by a specific infectious agent and which antigen may be used to elicit a specific immune response, e.g., antibody or cell-mediated immune response against the infectious agent such as a virus. Typically the antigen will comprise a moiety, e.g., polypeptide or glycoprotein expressed on the surface of the virus or other infectious agent, such as a capsid protein or other membrane protein.

“Inhibitory signal,” as used herein, refers broadly to a signal transmitted via an inhibitory receptor molecule on an immune cell. A signal antagonizes a signal via an activating receptor (e.g., via a TCR, CD3, BCR, or Fc molecule) and can result, e.g., in inhibition of: second messenger generation; proliferation; or effector function in the immune cell, e.g., reduced phagocytosis, antibody production, or cellular cytotoxicity, or the failure of the immune cell to produce mediators (e.g., cytokines (such as IL-2 or TNF-α) and/or mediators of allergic responses); or the development of anergy.

“Isolated,” as used herein, refers broadly to material removed from its original environment in which it naturally occurs, and thus is altered by the hand of man from its natural environment. Isolated material may be, for example, exogenous nucleic acid included in a vector system, exogenous nucleic acid contained within a host cell, or any material which has been removed from its original environment and thus altered by the hand of man (e.g., “isolated antibody”). For example, “isolated” or “purified,” as used herein, refers broadly to a protein, DNA, antibody, RNA, or biologically active portion thereof, that is substantially free of cellular material or other contaminating proteins from the cell or tissue source from which the biological substance is derived, or substantially free from chemical precursors or other chemicals when chemically synthesized. As used herein the term “isolated” refers to a compound of interest (for example a polynucleotide or a polypeptide) that is in an environment different from that in which the compound naturally occurs e.g. separated from its natural milieu such as by concentrating a peptide to a concentration at which it is not found in nature. “Isolated” includes compounds that are within samples that are substantially enriched for the compound of interest and/or in which the compound of interest is partially or substantially purified.

“Isolated antibody”, as used herein, is intended to refer to an antibody that is substantially free of other antibodies having different antigenic specificities (e.g., an isolated antibody that specifically binds VSTM5 is substantially free of antibodies that specifically bind antigens other than VSTM5). Moreover, an isolated antibody may be substantially free of other cellular material and/or chemicals.

“Isotype” herein refers to the antibody class (e.g., IgM or IgG1) that is encoded by the heavy chain constant region genes.

“K-assoc” or “Kₐ”, as used herein, refers broadly to the association rate of a particular antibody-antigen interaction, whereas the term “K_{diss}” or “Kd,” as used herein, refers to the dissociation rate of a particular antibody-antigen interaction. The term “K_{D}”, as used herein, is intended to refer to the dissociation constant, which is obtained from the ratio of K_{d} to Kₐ (i.e., Kd/Kₐ) and is expressed as a molar concentration (M). K_{D} values for antibodies can be determined using methods well established in the art such as plasmon resonance (Biacore®), ELISA and KINEXA. A preferred method for determining the K_{D} of an antibody is by using surface plasmon resonance, preferably using a biosensor system such as a Biacore® system or by ELISA.

“Label” or a “detectable moiety” as used herein, refers broadly to a composition detectable by spectroscopic, photochemical, biochemical, immunochemical, chemical, or other physical means.

“Low stringency,” “medium stringency,” “high stringency,” or “very high stringency conditions,” as used herein, refers broadly to conditions for nucleic acid hybridization and washing. Guidance for performing hybridization reactions can be found in Ausubel, et al. (2002) Short Protocols in Molecular Biology (5th Ed.) John Wiley & Sons, NY. Exemplary specific hybridization conditions include but are not limited to: (1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions in 6×SSC at about 45° C., followed by one or more washes in 0.2×SSC, 0.1% SDS at 60° C.; (3) high stringency hybridization conditions in 6×SSC at about 45° C., followed by one or more washes in 0.2×.SSC, 0.1% SDS at 65° C.; and (4) very high stringency hybridization conditions are 0.5M sodium phosphate, 7% SDS at 65° C., followed by one or more washes at 0.2×SSC, 1% SDS at 65° C.

“Mammal,” as used herein, refers broadly to any and all warm-blooded vertebrate animals of the class Mammalia, including humans, characterized by a covering of hair on the skin and, in the female, milk-producing mammary glands for nourishing the young. Examples of mammals include but are not limited to alpacas, armadillos, capybaras, cats, camels, chimpanzees, chinchillas, cattle, dogs, goats, gorillas, hamsters, horses, humans, lemurs, llamas, mice, non-human primates, pigs, rats, sheep, shrews, squirrels, tapirs, and voles. Mammals include but are not limited to bovine, canine, equine, feline, murine, ovine, porcine, primate, and rodent species. Mammal also includes any and all those listed on the Mammal Species of the World maintained by the National Museum of Natural History, Smithsonian Institution in Washington D.C.

“Multiple sclerosis” includes by way of example multiple sclerosis, benign multiple sclerosis, relapsing remitting multiple sclerosis, secondary progressive multiple sclerosis, primary progressive multiple sclerosis, progressive relapsing multiple sclerosis, chronic progressive multiple sclerosis, transitional/progressive multiple sclerosis, rapidly worsening multiple sclerosis, clinically-definite multiple sclerosis, malignant multiple sclerosis, also known as Marburg's Variant, and acute multiple sclerosis. Optionally, “conditions relating to multiple sclerosis” include, e.g., Devic's disease, also known as Neuromyelitis Optica; acute disseminated encephalomyelitis, acute demyelinating optic neuritis, demyelinative transverse myelitis, Miller-Fisher syndrome, encephalomyeloradiculoneuropathy, acute demyelinative polyneuropathy, tumefactive multiple sclerosis and Balo's concentric sclerosis.

“Naturally-occurring nucleic acid molecule,” as used herein, refers broadly refers to an RNA or DNA molecule having a nucleotide sequence that occurs in nature (e.g., encodes a natural protein).

“Nucleic acid” or “nucleic acid sequence,” as used herein, refers broadly to a deoxy-ribonucleotide or ribonucleotide oligonucleotide in either single- or double-stranded form. The term encompasses nucleic acids, i.e., oligonucleotides, containing known analogs of natural nucleotides. The term also encompasses nucleic-acid-like structures with synthetic backbones. Unless otherwise indicated, a particular nucleic acid sequence also implicitly encompasses conservatively modified variants thereof (e.g., degenerate codon substitutions) and complementary sequences, as well as the sequence explicitly indicated. The term nucleic acid is used interchangeably with gene, cDNA, mRNA, oligonucleotide, and polynucleotide.

“Oligomerization domain”, as used herein, refers broadly to a domain that when attached to a VSTM5 extracellular domain or fragment thereof, facilitates oligomerization. Said oligomerization domains comprise self-associating α-helices, for example, leucine zippers, that can be further stabilized by additional disulfide bonds. The domains are designed to be compatible with vectorial folding across a membrane, a process thought to facilitate in vivo folding of the polypeptide into a functional binding protein. Examples thereof are known in the art and include by way of example coiled GCN4, and COMP. The α-helical coiled coil is probably the most widespread subunit oligomerization motif found in proteins. Accordingly, coiled coils fulfill a variety of different functions. In several families of transcriptional activators, for example, short leucine zippers play an important role in positioning the DNA-binding regions on the DNA. Ellenberger, et al. (1992) Cell 71: 1223-1237. Coiled coils are also used to form oligomers of intermediate filament proteins. Coiled-coil proteins furthermore appear to play an important role in both vesicle and viral membrane fusion. Skehel and Wiley (1998) Cell 95: 871-874. In both cases hydrophobic sequences, embedded in the membranes to be fused, are located at the same end of the rod-shaped complex composed of a bundle of long α-helices. This molecular arrangement is believed to cause close membrane apposition as the complexes are assembled for membrane fusion. The coiled coil is often used to control oligomerization. It is found in many types of proteins, including transcription factors include, but not limited to GCN4, viral fusion peptides, SNARE complexes and certain tRNA synthetases, among others. Very long coiled coils are found in proteins such as tropomyosin, intermediate filaments and spindle-pole-body components. Coiled coils involve a number of α-helices that are supercoiled around each other in a highly organized manner that associate in a parallel or an antiparallel orientation. Although dimers and trimers are the most common. The helices may be from the same or from different proteins. The coiled-coil is formed by component helices coming together to bury their hydrophobic seams. As the hydrophobic seams twist around each helix, so the helices also twist to coil around each other, burying the hydrophobic seams and forming a supercoil. It is the characteristic interdigitation of side chains between neighboring helices, known as knobs-into-holes packing, that defines the structure as a coiled coil. The helices do not have to run in the same direction for this type of interaction to occur, although parallel conformation is more common Antiparallel conformation is very rare in trimers and unknown in pentamers, but more common in intramolecular dimers, where the two helices are often connected by a short loop. In the extracellular space, the heterotrimeric coiled-coil protein laminin plays an important role in the formation of basement membranes. Other examples are the thrombospondins and cartilage oligomeric matrix protein (COMP) in which three (thrombospondins 1 and 2) or five (thrombospondins 3, 4 and COMP) chains are connected. The molecules have a flower bouquet-like appearance, and the reason for their oligomeric structure is probably the multivalent interaction of the C-terminal domains with cellular receptors. The yeast transcriptional activator GCN4 is 1 of over 30 identified eukaryotic proteins containing the basic region leucine zipper (bZIP) DNA-binding motif. Ellenberger, et al. (1992) Cell 71: 1223-1237. The bZIP dimer is a pair of continuous α helices that form a parallel coiled-coil over their carboxy-terminal 34 residues and gradually diverge toward their amino termini to pass through the major groove of the DNA binding site. The coiled-coil dimerization interface is oriented almost perpendicular to the DNA axis, giving the complex the appearance of the letter T. bZIP contains a 4-3 heptad repeat of hydrophobic and nonpolar residues that pack together in a parallel α-helical coiled-coil. Ellenberger, et al. (1992) Cell 71: 1223-1237. The stability of the dimer results from the side-by-side packing of leucines and nonpolar residues in positions a and d of the heptad repeat, as well as a limited number of intra- and interhelical salt bridges, shown in a crystal structure of the GCN4 leucine zipper peptide. Ellenberger, et al. (1992) Cell 71: 1223-1237. Another example is CMP (matrilin-1) isolated from bovine tracheal cartilage as a homotrimer of subunits of Mr 52,000 (Paulsson & Heinegard (1981) Biochem J. 197: 367-375), where each subunit consists of a vWFA1 module, a single EGF domain, a vWFA2 module and a coiled coil domain spanning five heptads. Kiss, et al. (1989) J. Biol. Chem. 264:8126-8134; Hauser and Paulsson (1994) J. Biol. Chem. 269: 25747-25753. Electron microscopy of purified CMP showed a bouquet-like trimer structure in which each subunit forms an ellipsoid emerging from a common point corresponding to the coiled coil. Hauser and Paulsson (1994) J. Biol. Chem. 269: 25747-25753. The coiled coil domain in matrilin-1 has been extensively studied. The trimeric structure is retained after complete reduction of interchain disulfide bonds under non-denaturing conditions. Hauser and Paulsson (1994) J. Biol. Chem. 269: 25747-25753. Yet another example is Cartilage Oligomeric Matrix Protein (COMP). A non-collagenous glycoprotein, COMP, was first identified in cartilage. Hedbom, et al. (1992) J. Biol. Chem. 267:6132-6136. The protein is a 524 kDa homopentamer of five subunits which consists of an N-terminal heptad repeat region (cc) followed by four epidermal growth factor (EGF)-like domains (EF), seven calcium-binding domains (T3) and a C-terminal globular domain (TC). According to this domain organization, COMP belongs to the family of thrombospondins. Heptad repeats (abcdefg)ₙ with preferentially hydrophobic residues at positions a and d form-helical coiled-coil domains. Cohen and Parry (1994) Science 263: 488-489. Recently, the recombinant five-stranded coiled-coil domain of COMP (COMPcc) was crystallized and its structure was solved at 0.2 nm resolution. Malashkevich, et al. (1996) Science 274: 761-765.

“Operatively linked”, as used herein, refers broadly to when two DNA fragments are joined such that the amino acid sequences encoded by the two DNA fragments remain in-frame.

“Paratope,” as used herein, refers broadly to the part of an antibody which recognizes an antigen (e.g., the antigen-binding site of an antibody.) Paratopes may be a small region (e.g., 15-22 amino acids) of the antibody's Fv region and may contain parts of the antibody's heavy and light chains. See Goldsby, et al. Antigens (Chapter 3) Immunology (5^{th} Ed.) New York: W.H. Freeman and Company, pages 57-75.

“Patient,” or “subject” or “recipient” or “treated individual” are used interchangeably herein, and refers broadly to any animal that is in need of treatment either to alleviate a disease state or to prevent the occurrence or reoccurrence of a disease state. Also, “Patient” as used herein, refers broadly to any animal that has risk factors, a history of disease, susceptibility, symptoms, and signs, was previously diagnosed, is at risk for, or is a member of a patient population for a disease. The patient may be a clinical patient such as a human or a veterinary patient such as a companion, domesticated, livestock, exotic, or zoo animal. The term “subject” may be used interchangeably with the term “patient.”

“Polypeptide,” “peptide” and “protein”, are used interchangeably and refer broadly to a polymer of amino acid residues of any length, regardless of modification (e.g., phosphorylation or glycosylation). The terms apply to amino acid polymers in which one or more amino acid residue is an analog or mimetic of a corresponding naturally occurring amino acid, as well as to naturally occurring amino acid polymers. The terms apply to amino acid polymers in which one or more amino acid residue is an artificial chemical mimetic of a corresponding naturally occurring amino acid, as well as to naturally occurring amino acid polymers and non-naturally occurring amino acid polymer. Polypeptides can be modified, e.g., by the addition of carbohydrate residues to form glycoproteins. The terms “polypeptide,” “peptide” and “protein” expressly include glycoproteins, as well as non-glycoproteins.

“Promoter,” as used herein, refers broadly to an array of nucleic acid sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation.

“Prophylactically effective amount,” as used herein, refers broadly to the amount of a compound that, when administered to a patient for prophylaxis of a disease or prevention of the reoccurrence of a disease, is sufficient to effect such prophylaxis for the disease or reoccurrence. The prophylactically effective amount may be an amount effective to prevent the incidence of signs and/or symptoms. The “prophylactically effective amount” may vary depending on the disease and its severity and the age, weight, medical history, predisposition to conditions, preexisting conditions, of the patient to be treated.

“Prophylactic vaccine” and/or “Prophylactic vaccination” refers to a vaccine used to prevent a disease or symptoms associated with a disease such as cancer or an infectious condition.

“Prophylaxis” as used herein, refers broadly to a course of therapy where signs and/or symptoms are not present in the patient, are in remission, or were previously present in a patient. Prophylaxis includes preventing disease occurring subsequent to treatment of a disease in a patient. Further, prevention includes treating patients who may potentially develop the disease, especially patients who are susceptible to the disease (e.g., members of a patent population, those with risk factors, or at risk for developing the disease).

“Psoriasis” herein includes one or more of psoriasis, Nonpustular Psoriasis including Psoriasis vulgaris and Psoriatic erythroderma (erythrodermic psoriasis), Pustular psoriasis including Generalized pustular psoriasis (pustular psoriasis of von Zumbusch), Pustulosis palmaris et plantaris (persistent palmoplantar pustulosis, pustular psoriasis of the Barber type, pustular psoriasis of the extremities), Annular pustular psoriasis, Acrodermatitis continua, Impetigo herpetiformis. Optionally, conditions relating to psoriasis include, e.g., drug-induced psoriasis, Inverse psoriasis, Napkin psoriasis, Seborrheic-like psoriasis, Guttate psoriasis, Nail psoriasis, and Psoriatic arthritis.

“Recombinant” as used herein, refers broadly with reference to a product, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

“Rheumatoid arthritis” includes by way of example rheumatoid arthritis, gout and pseudo-gout, juvenile idiopathic arthritis, juvenile rheumatoid arthritis, Still's disease, ankylosing spondylitis, rheumatoid vasculitis, as well as other conditions relating to rheumatoid arthritis such as e.g., osteoarthritis, sarcoidosis, Henoch-Schönlein purpura, Psoriatic arthritis, Reactive arthritis, Spondyloarthropathy, septic arthritis, Hemochromatosis, Hepatitis, vasculitis, Wegener's granulomatosis, Lyme disease, Familial Mediterranean fever, Hyperimmunoglobulinemia D with recurrent fever, TNF receptor associated periodic syndrome, and Enteropathic arthritis associated with inflammatory bowel disease.

“Signal sequence” or “signal peptide,” as used herein, refers broadly to a peptide containing about 15 or more amino acids which occurs at the N-terminus of secretory and membrane bound polypeptides and which contains a large number of hydrophobic amino acid residues. For example, a signal sequence contains at least about 10-30 amino acid residues, preferably about 15-25 amino acid residues, more preferably about 18-20 amino acid residues, and even more preferably about 19 amino acid residues, and has at least about 35-65%, preferably about 38-50%, and more preferably about 40-45% hydrophobic amino acid residues (e.g., Valine, Leucine, Isoleucine or Phenylalanine). A “signal sequence,” also referred to in the art as a “signal peptide,” serves to direct a polypeptide containing such a sequence to a lipid bilayer, and is cleaved in secreted.

“Sjögren's syndrome” herein includes one or more of Sjögren's syndrome, Primary Sjögren's syndrome and Secondary Sjögren's syndrome, as well as conditions relating to Sjögren's syndrome including connective tissue disease, such as rheumatoid arthritis, systemic lupus erythematosus, or scleroderma. Other complications include pneumonia, pulmonary fibrosis, interstitial nephritis, inflammation of the tissue around the kidney's filters, glomerulonephritis, renal tubular acidosis, carpal tunnel syndrome, peripheral neuropathy, cranial neuropathy, primary biliary cirrhosis (PBC), cirrhosis, Inflammation in the esophagus, stomach, pancreas, and liver (including hepatitis), Polymyositis, Raynaud's phenomenon, Vasculitis, Autoimmune thyroid problems, and lymphoma.

“Specifically (or selectively) binds” to an antibody or “specifically (or selectively) immunoreactive with,” or “specifically interacts or binds,” as used herein, refers broadly to a protein or peptide (or other epitope), refers, in some embodiments, to a binding reaction that is determinative of the presence of the protein in a heterogeneous population of proteins and other biologics. For example, under designated immunoassay conditions, the specified antibodies bind to a particular protein at least two times greater than the background (non-specific signal) and do not substantially bind in a significant amount to other proteins present in the sample. Typically a specific or selective reaction will be at least twice background signal or noise and more typically more than about 10 to 100 times background.

“Specifically hybridizable” and “complementary” as used herein, refer broadly to a nucleic acid can form hydrogen bond(s) with another nucleic acid sequence by either traditional Watson-Crick or other non-traditional types. The binding free energy for a nucleic acid molecule with its complementary sequence is sufficient to allow the relevant function of the nucleic acid to proceed, e.g., RNAi activity. Determination of binding free energies for nucleic acid molecules is well known in the art. See, e.g., Turner, et al. (1987) CSH Symp. Quant. Biol. LII: 123-33; Frier, et al. (1986) PNAS 83: 9373-77; Turner, et al. (1987) J. Am. Chem. Soc. 109: 3783-85. A percent complementarity indicates the percentage of contiguous residues in a nucleic acid molecule that can form hydrogen bonds (e.g., Watson-Crick base pairing) with a second nucleic acid sequence (e.g., about at least 5, 6, 7, 8, 9, 10 out of 10 being about at least 50%, 60%, 70%, 80%, 90%, and 100% complementary, inclusive). “Perfectly complementary” or 100% complementarity refers broadly all of the contiguous residues of a nucleic acid sequence hydrogen bonding with the same number of contiguous residues in a second nucleic acid sequence.

“Substantial complementarity” refers to polynucleotide strands exhibiting about at least 90% complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically may differ by at least 5 nucleotides.

“Signs” of disease, as used herein, refers broadly to any abnormality indicative of disease, discoverable on examination of the patient; an objective indication of disease, in contrast to a symptom, which is a subjective indication of disease.

“Solid support,” “support,” and “substrate,” as used herein, refers broadly to any material that provides a solid or semi-solid structure with which another material can be attached including but not limited to smooth supports (e.g., metal, glass, plastic, silicon, and ceramic surfaces) as well as textured and porous materials.

“Subject” or “patient” includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc., i.e., anyone suitable to be treated according to the present invention include, but are not limited to, avian and mammalian subjects, and are preferably mammalian. Any mammalian subject in need of being treated according to the present invention is suitable. Human subjects of both genders and at any stage of development (i.e., neonate, infant, juvenile, adolescent, adult) can be treated according to the present invention. The present invention may also be carried out on animal subjects, particularly mammalian subjects such as mice, rats, dogs, cats, cattle, goats, sheep, and horses for veterinary purposes, and for drug screening and drug development purposes. “Subjects” is used interchangeably with “patients.”

“Substantially free of chemical precursors or other chemicals,” as used herein, refers broadly to preparations of VSTM5 protein in which the protein is separated from chemical precursors or other chemicals which are involved in the synthesis of the protein. In one embodiment, the language “substantially free of chemical precursors or other chemicals” includes preparations of VSTM5 protein having less than about 30% (by dry weight) of chemical precursors or non-VSTM5 chemicals, more preferably less than about 20% chemical precursors or non-VSTM5 chemicals, still more preferably less than about 10% chemical precursors or non-VSTM5 chemicals, and most preferably less than about 5% chemical precursors or non-VSTM5 chemicals.

“Symptoms” of disease as used herein, refers broadly to any morbid phenomenon or departure from the normal in structure, function, or sensation, experienced by the patient and indicative of disease.

“Systemic lupus erythematosus”, as used herein comprises one or more of systemic lupus erythematosus, discoid lupus, lupus arthritis, lupus pneumonitis, lupus nephritis. Conditions relating to systemic lupus erythematosus include osteoarticular tuberculosis, antiphospholipid antibody syndrome, inflammation of various parts of the heart, such as pericarditis, myocarditis, and endocarditis, Lung and pleura inflammation, pleuritis, pleural effusion, chronic diffuse interstitial lung disease, pulmonary hypertension, pulmonary emboli, pulmonary hemorrhage, and shrinking lung syndrome, lupus headache, Guillain-Barré syndrome, aseptic meningitis, demyelinating syndrome, mononeuropathy, mononeuritis multiplex, myasthenia gravis, myelopathy, cranial neuropathy, polyneuropathy, and vasculitis.

“T cell,” as used herein, refers broadly to CD4⁺ T cells and CD8⁺ T cells. The term T cell also includes both T helper 1 type T cells and T helper 2 type T cells.

“Therapy,” “therapeutic,” “treating,” or “treatment”, as used herein, refers broadly to treating a disease, arresting, or reducing the development of the disease or its clinical symptoms, and/or relieving the disease, causing regression of the disease or its clinical symptoms. Therapy encompasses prophylaxis, treatment, remedy, reduction, alleviation, and/or providing relief from a disease, signs, and/or symptoms of a disease. Therapy encompasses an alleviation of signs and/or symptoms in patients with ongoing disease signs and/or symptoms (e.g., inflammation, pain). Therapy also encompasses “prophylaxis”. The term “reduced”, for purpose of therapy, refers broadly to the clinical significant reduction in signs and/or symptoms. Therapy includes treating relapses or recurrent signs and/or symptoms (e.g., inflammation, pain). Therapy encompasses but is not limited to precluding the appearance of signs and/or symptoms anytime as well as reducing existing signs and/or symptoms and eliminating existing signs and/or symptoms. Therapy includes treating chronic disease (“maintenance”) and acute disease. For example, treatment includes treating or preventing relapses or the recurrence of signs and/or symptoms (e.g., inflammation, pain).

“Therapeutic vaccine” and/or “therapeutic vaccination” refers to a vaccine used to treat a disease such as cancer or an infectious condition.

“Treg cell” (sometimes also referred to as suppressor T cells or inducible Treg cells or iTregs) as used herein refers to a subpopulation of T cells which modulate the immune system and maintain tolerance to self-antigens and can abrogate autoimmune diseases. Foxp3⁺ CD4⁺CD25⁺ regulatory T cells (Tregs) are critical in maintaining peripheral tolerance under normal immunity.

“Transmembrane domain,” as used herein, refers broadly to an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an α-helical structure. In an embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta, et al. (1996) Annu. Rev. Neurosci. 19:235-263.

“Transgenic animal,” as used herein, refers broadly to a non-human animal, preferably a mammal, more preferably a mouse, in which one or more of the cells of the animal includes a “transgene”. The term “transgene” refers to exogenous DNA which is integrated into the genome of a cell from which a transgenic animal develops and which remains in the genome of the mature animal, for example directing the expression of an encoded gene product in one or more cell types or tissues of the transgenic animal.

“Tumor,” as used herein, refers broadly to at least one cell or cell mass in the form of a tissue neoformation, in particular in the form of a spontaneous, autonomous and irreversible excess growth, which is more or less disinhibited, of endogenous tissue, which growth is as a rule associated with the more or less pronounced loss of specific cell and tissue functions. This cell or cell mass is not effectively inhibited, in regard to its growth, by itself or by the regulatory mechanisms of the host organism, e.g., colorectal cancer, melanoma or carcinoma. Tumor antigens not only include antigens present in or on the malignant cells themselves, but also include antigens present on the stromal supporting tissue of tumors including endothelial cells and other blood vessel components.

“Type 1 diabetes” herein includes one or more of type 1 diabetes, insulin-dependent diabetes mellitus, idiopathic diabetes, juvenile type 1 diabetes, maturity onset diabetes of the young, latent autoimmune diabetes in adults, gestational diabetes. Conditions relating to type 1 diabetes include, neuropathy including polyneuropathy, mononeuropathy, peripheral neuropathy and autonomicneuropathy; eye complications: glaucoma, cataracts, and retinopathy.

“Unresponsiveness,” as used herein, refers broadly to refractivity of immune cells to stimulation, e.g., stimulation via an activating receptor or a cytokine. Unresponsiveness can occur, e.g., because of exposure to immunosuppressants or high doses of antigen.

“Uveitis” as used herein comprises one or more of uveitis, anterior uveitis (or iridocyclitis), intermediate uveitis (pars planitis), posterior uveitis (or chorioretinitis) and the panuveitic form.

“Vaccine” as used herein, refers to a biological preparation that as improves immunity to a particular disease, e.g., cancer or an infectious disease, wherein the vaccine includes a disease specific antigen, e.g., a cancer antigen or infectious agent antigen, against which immune responses are elicited. A vaccine typically includes an adjuvant as immune potentiator to stimulate the immune system. This includes prophylactic (which prevent disease) and therapeutic vaccines (which treat the disease or its symptoms).

“Variable region” or “VR,” as used herein, refers broadly to the domains within each pair of light and heavy chains in an antibody that are involved directly in binding the antibody to the antigen. Each heavy chain has at one end a variable domain (V_{H}) followed by a number of constant domains. Each light chain has a variable domain (V_{L}) at one end and a constant domain at its other end; the constant domain of the light chain is aligned with the first constant domain of the heavy chain, and the light chain variable domain is aligned with the variable domain of the heavy chain.

“Vector,” as used herein, refers broadly to a nucleic acid molecule capable of transporting another nucleic acid molecule to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. The techniques and procedures are generally performed according to conventional methods well known in the art and as described in various general and more specific references that are cited and discussed throughout the present specification. See, e.g., Sambrook, et al. (2001) Molec. Cloning: Lab. Manual [3rd Ed] Cold Spring Harbor Laboratory Press. Standard techniques may be used for recombinant DNA, oligonucleotide synthesis, and tissue culture, and transformation (e.g., electroporation, lipofection). Enzymatic reactions and purification techniques may be performed according to manufacturer's specifications or as commonly accomplished in the art or as described herein.

Having defined certain terms and phrases used in the present application, specific types of anti-VSTM5 antibodies, antigen-binding fragments, and conjugates thereof, and methods for the production and use thereof which are embraced by the invention are further described below.

Antibodies Having Particular Germline Sequences

In certain embodiments, an anti-VSTM5 antibody according to the invention comprises a heavy chain variable region from a particular germline heavy chain immunoglobulin gene and/or a light chain variable region from a particular germline light chain immunoglobulin gene. For example, such anti-VSTM5 antibody may comprise or consist of a human antibody comprising heavy or light chain variable regions that are the product of or “derived from” a particular germline sequence if the variable regions of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody.

A human antibody that is the product of or “derived from” a particular human germline immunoglobulin sequence may contain amino acid differences as compared to the germline sequence, due to, for example, naturally-occurring somatic mutations or intentional introduction of site-directed mutation. However, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95, 96, 97, 98 or 99%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

Homologous Antibodies

In certain embodiments, an anti-VSTM5 antibody according to the invention comprises heavy and light chain variable regions comprising amino acid sequences that are homologous to isolated anti-VSTM5 amino acid sequences of preferred anti-VSTM5 antibodies, respectively, wherein the antibodies retain the desired functional properties of the parent anti-VSTM5 antibodies. As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available commercially), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J Mol. Biol. 215:403-10. BLAST protein searches can be performed with the XBLAST program, score=50, wordlength=3 to obtain amino acid sequences homologous to the antibody molecules according to at least some embodiments of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

Antibodies with Conservative Modifications

In certain embodiments, an anti-VSTM5 antibody according to the invention comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on preferred anti-anti-VSTM5 antibodies isolated and produced using methods herein, or conservative modifications thereof, and wherein the antibodies retain the desired functional properties of anti-VSTM5 antibodies according to at least some embodiments of the invention, respectively.

In various embodiments, the anti-VSTM5 antibody can be, for example, human antibodies, humanized antibodies or chimeric antibodies. As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody according to at least some embodiments of the invention by standard techniques known in the art, such as site-directed mutagenesis and PCR-mediated mutagenesis. Conservative amino acid substitutions are ones in which the amino acid residue is replaced with an amino acid residue having a similar side chain Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), β-branched side chains (e.g., threonine, valine, isoleucine) and aromatic side chains (e.g., tyrosine, phenylalanine, tryptophan, histidine). Thus, one or more amino acid residues within the CDR regions of an antibody according to at least some embodiments of the invention can be replaced with other amino acid residues from the same side chain family and the altered antibody can be tested for retained function (i.e., the functions set forth in (c) through (j) above) using the functional assays described herein.

Anti-VSTM5 Antibodies that Bind to the Same Epitope

In certain embodiments, an anti-VSTM5 antibody according to the invention possesses desired functional properties such as modulation of immune stimulation and related functions. Other antibodies with the same epitope specificity may be selected and will have the ability to cross-compete for binding to VSTM5 antigen with the desired antibodies. Alternatively, the epitopic specificity of a desired antibody may be determined using a library of overlapping peptides comprising the entire VSTM5 polypeptide, e.g., 15-mers or an overlapping peptide library constituting a portion containing a desired epitope of VSTM5 and antibodies which bind to the same peptides or one or more residues thereof in the library are determined to bind the same linear or conformational epitope.

Engineered and Modified Antibodies

In certain embodiments, an anti-VSTM5 antibody according to the invention can be prepared using an antibody having one or more of the V_{H} and/or V_{L} sequences derived from an anti-VSTM5 antibody starting material to engineer a modified antibody, which modified antibody may have altered properties from the starting antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant regions, for example to alter the effector functions of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L et al. (1998) Nature 332:323-327; Jones, P. et al. (1986) Nature 321:522-525; Queen, C. et al. (1989) Proc. Natl. Acad. See. U.S.A. 86:10029-10033; U.S. Pat. No. 5,225,539 to Winter, and U.S. Pat. Nos. 5,530,101; 5,585,089; 5,693,762 and 6,180,370 to Queen et al.)

Suitable framework sequences can be obtained from public DNA databases or published references that include germline antibody gene sequences. For example, germline DNA sequences for human heavy and light chain variable region genes can be found in the “VBase” human germline sequence database (available on the Internet), as well as in Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J. P. L. et al. (1994) “A Directory of Human Germ-line V_{H} Segments Reveals a Strong Bias in their Usage” Eur. J Immunol. 24:827-836; the contents of each of which are expressly incorporated herein by reference.

Another type of variable region modification is to mutate amino acid residues within the V_{H} and/or V_{L} CDR 1, CDR2 and/or CDR3 regions to thereby improve one or more binding properties (e.g., affinity) of the antibody of interest. Site-directed mutagenesis or PCR-mediated mutagenesis can be performed to introduce the mutations and the effect on antibody binding, or other functional property of interest, can be evaluated in appropriate in vitro or in vivo assays. Preferably conservative modifications (as discussed above) are introduced. The mutations may be amino acid substitutions, additions or deletions, but are preferably substitutions. Moreover, typically no more than one, two, three, four or five residues within a CDR region are altered.

Engineered antibodies according to at least some embodiments of the invention include those in which modifications have been made to framework residues within V_{H} and/or V_{L}, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived.

In addition or alternative to modifications made within the framework or CDR regions, antibodies according to at least some embodiments of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody according to at least some embodiments of the invention may be chemically modified (e.g., one or more chemical moieties can be attached to the antibody) or be modified to alter its glycosylation, again to alter one or more functional properties of the antibody. Such embodiments are described further below. The numbering of residues in the Fc region is that of the EU index of Kabat.

In one embodiment, the hinge region of C_{H1} is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half-life. Various approaches are possible. For example, one or more of the following mutations can be introduced: T252L, T254S, T256F, as described in U.S. Pat. No. 6,277,375 to Ward. Alternatively, to increase the biological half-life, the antibody can be altered within the C_{H1} or C_{L} region to contain a salvage receptor binding epitope taken from two loops of a C_{H2} domain of an Fc region of an IgG, as described in U.S. Pat. Nos. 5,869,046 and 6,121,022 by Presta et al.

In yet other embodiments, the Fc region is altered by replacing at least one amino acid residue with a different amino acid residue to alter the effector functions of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region is modified to increase the ability of the antibody to mediate antibody dependent cellular cytotoxicity (ADCC) and/or to increase the affinity of the antibody for an Fcγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for FcγRI, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 are shown to improve binding to FcγRIII. Additionally, the following combination mutants are shown to improve FcγRIII binding: T256A/S298A, S298A/E333A, S298A/K224A and S298A/E333A/K334A. Furthermore, mutations such as M252Y/S254T/T256E or M428L/N434S improve binding to FcRn and increase antibody circulation half-life (see Chan C A and Carter P J (2010) Nature Rev Immunol 10:301-316).

In still another embodiment, the antibody can be modified to abrogate in vivo Fab arm exchange. Specifically, this process involves the exchange of IgG4 half-molecules (one heavy chain plus one light chain) between other IgG4 antibodies that effectively results in bispecific antibodies which are functionally monovalent. Mutations to the hinge region and constant domains of the heavy chain can abrogate this exchange (see Aalberse, R C, Schuurman J., 2002, Immunology 105:9-19).

In still another embodiment, the glycosylation of an antibody is modified. For example, an aglycosylated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglyclosylation may increase the affinity of the antibody for antigen. Such an approach is described in further detail in U.S. Pat. Nos. 5,714,350 and 6,350,861 by Co et al.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies according to at least some embodiments of the invention to thereby produce an antibody with altered glycosylation. For example, the cell lines Ms704, Ms705, and Ms709 lack the fucosyltransferase gene, FUT8 (α(1,6) fucosyltransferase), such that antibodies expressed in the Ms704, Ms705, and Ms709 cell lines lack fucose on their carbohydrates. The Ms704, Ms705, and Ms709 FUT8 cell lines are created by the targeted disruption of the FUT8 gene in CHO/DG44 cells using two replacement vectors (see U.S. Patent Publication No. 20040110704 by Yamane et al. and Yamane-Ohnuki et al. (2004) Biotechnol Bioeng 87:614-22). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the a 1,6 bond-related enzyme. Hanai et al. also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., β(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180). Alternatively, the fucose residues of the antibody may be cleaved off using a fucosidase enzyme. For example, the fucosidase α-L-fucosidase removes fucosyl residues from antibodies (Tarentino, A. L. et al. (1975) Biochem. 14:5516-23).

Another modification of the antibodies herein that is contemplated by the invention is pegylation or the addition of other water soluble moieties, typically polymers, e.g., in order to enhance half-life. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies according to at least some embodiments of the invention. See for example, EP 0 154 316 by Nishimura et al. and EP 0 401 384 by Ishikawa et al.

Methods of Engineering Antibodies

In certain embodiments, an anti-VSTM5 antibody according to the invention having V_{H} and V_{L} sequences can be used to create new anti-VSTM5 antibodies, respectively, by modifying the V_{H} and/or V_{L} sequences, or the constant regions attached thereto. Thus, in another aspect according to at least some embodiments of the invention, the structural features of an anti-VSTM5 antibody according to at least some embodiments of the invention, are used to create structurally related anti-VSTM5 antibodies that retain at least one functional property of the antibodies according to at least some embodiments of the invention, such as binding to human VSTM5. For example, one or more CDR regions of one VSTM5 antibody or mutations thereof can be combined recombinantly with known framework regions and/or other CDRs to create additional, recombinantly-engineered, anti-VSTM5 antibodies according to at least some embodiments of the invention, as discussed above. Other types of modifications include those described in the previous section. The starting material for the engineering method is one or more of the V_{H} and/or V_{L} sequences provided herein, or one or more CDR regions thereof. To create the engineered antibody, it is not necessary to actually prepare (i.e., express as a protein) an antibody having one or more of the V_{H} and/or V_{L} sequences provided herein, or one or more CDR regions thereof. Rather, the information contained in the sequences is used as the starting material to create a “second generation” sequences derived from the original sequences and then the “second generation” sequences is prepared and expressed as a protein.

Standard molecular biology techniques can be used to prepare and express altered antibody sequence. Preferably, the anti-VSTM5 antibody encoded by the altered antibody sequences is one that retains one, some or all of the functional properties of the anti-VSTM5 antibodies, respectively, produced by methods and with sequences provided herein, which functional properties include binding to VSTM5 antigen with a specific KD level or less and/or modulating immune responses and/or selectively binding to desired target cells such as for example, that express VSTM5 antigen.

The functional properties of the altered antibodies can be assessed using standard assays available in the art and/or described herein. In certain embodiments of the methods of engineering antibodies according to at least some embodiments of the invention, mutations can be introduced randomly or selectively along all or part of an anti-VSTM5 antibody coding sequence and the resulting modified anti-VSTM5 antibodies can be screened for binding activity and/or other desired functional properties.

Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

Nucleic Acid Molecules Encoding Antibodies

The invention further provides nucleic acids which encode an anti-VSTM5 antibody according to the invention, or a fragment or conjugate thereof. The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. (1987) Current Protocols in Molecular Biology, Greene Publishing and Wiley Interscience, New York. A nucleic acid according to at least some embodiments of the invention can be, for example, DNA or RNA and may or may not contain intronic sequences. In a preferred embodiment, the nucleic acid is a cDNA molecule.

Nucleic acids according to at least some embodiments of the invention can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

Once DNA fragments encoding V_{H} and V_{L} segments are obtained, these DNA fragments can be further manipulated by standard recombinant DNA techniques, for example to convert the variable region genes to full-length antibody chain genes, to Fab fragment genes or to a scFv gene. In these manipulations, a V_{L}- or V_{H}-encoding DNA fragment is operatively linked to another DNA fragment encoding another protein, such as an antibody constant region or a flexible linker. As previously defined, “operatively linked”, means that that the two DNA fragments are joined such that the amino acid sequences encoded by the two DNA fragments remain in-frame.

The isolated DNA encoding the V_{H} region can be converted to a full-length heavy chain gene by operatively linking the V_{H}-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1, IgG2 or IgG4 constant region. For a Fab fragment heavy chain gene, the V_{H}-encoding DNA can be operatively linked to another DNA molecule encoding only the heavy chain C_{H1} constant region.

The isolated DNA encoding the V_{L} region can be converted to a full-length light chain gene (as well as a Fab light chain gene) by operatively linking the V_{L}-encoding DNA to another DNA molecule encoding the light chain constant region, C_{L}. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa (κ) or lambda constant region, but most preferably is a κ constant region.

To create a scFv gene, the V_{H}- and V_{L}-encoding DNA fragments are operatively linked to another fragment encoding a flexible linker, e.g., encoding the amino acid sequence (Gly4-Ser)3, such that the V_{H} and V_{L} sequences can be expressed as a contiguous single-chain protein, with the V_{L} and V_{H} regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

Production of Anti-VSTM5 Monoclonal Antibodies

Anti-VSTM5 monoclonal antibodies (mAbs) and antigen-binding fragments according to the present invention can be produced by a variety of techniques, including conventional monoclonal antibody methodology e.g., the standard somatic cell hybridization technique of Kohler and Milstein (1975) Nature 256:495. Although somatic cell hybridization procedures are preferred, in principle, other techniques for producing monoclonal antibody can be employed e.g., viral or oncogenic transformation of B lymphocytes.

A preferred animal system for preparing hybridomas is the murine system. Hybridoma production in the mouse is a very well-established procedure Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art (see e.g., U.S. Pat. No. 5,225,539 to Winter and U.S. Pat. Nos. 5,530,101; 5,585,089; 5,693,762 and 6,180,370 to Queen et al.).

According to at least some embodiments of the invention, the antibodies are human monoclonal antibodies. Such human monoclonal antibodies directed against VSTM5 can be generated using transgenic or transchromosomic mice carrying parts of the human immune system rather than the mouse system. These transgenic and transchromosomic mice include mice referred to herein as the HuMAb Mouse™ and KM Mouse™, respectively, and are collectively referred to herein as “human Ig mice.” The HuMAb Mouse™ (Medarex Inc.) contains human immunoglobulin gene miniloci that encode unrearranged human heavy μ and γ and κ light chain immunoglobulin sequences, together with targeted mutations that inactivate the endogenous μ and κ chain loci (see e.g., Lonberg, et al. (1994) Nature 368(6474): 856-859). Accordingly, the mice exhibit reduced expression of mouse IgM or κ and in response to immunization, the introduced human heavy and light chain transgenes undergo class switching and somatic mutation to generate high affinity human IgG κ monoclonal (Lonberg, N. et al. (1994), supra; reviewed in Lonberg, N. (1994) Handbook of Experimental Pharmacology 113:49-101; Lonberg, N. and Huszar, D. (1995) Intern. Rev. Immunol. 13: 65-93, and Harding, F. and Lonberg, N. (1995) Ann. N.Y. Acad. Sci. 764:536-546). The preparation and use of the HuMab Mouse®, and the genomic modifications carried by such mice, is further described in Taylor, L. et al. (1992) Nucleic Acids Research 20:6287-6295; Chen, J. et al. (1993) International Immunology 5:647-656; Tuaillon et al. (1993) Proc. Natl. Acad. Sci. USA 90:3720-3724; Choi et al. (1993) Nature Genetics 4:117-123; Chen, J. et al. (1993) EMBO J. 12: 821-830; Tuaillon et al. (1994) J. Immunol. 152:2912-2920; Taylor, L. et al. (1994) International Immunology 6:579-591; and Fishwild, D. et al. (1996) Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al.

In another embodiment, human antibodies according to at least some embodiments of the invention can be raised using a mouse that carries human immunoglobulin sequences on transgenes and transchomosomes, such as a mouse that carries a human heavy chain transgene and a human light chain transchromosome. Such mice, referred to herein as “KM Mice™”, are described in detail in PCT Publication WO 02/43478 to Ishida et al.

Still further, alternative transgenic animal systems expressing human immunoglobulin genes are available in the art and can be used to raise anti-VSTM5 antibodies according to at least some embodiments of the invention. For example, an alternative transgenic system referred to as the Xenomouse (Abgenix, Inc.) can be used; such mice are described in, for example, U.S. Pat. Nos. 5,939,598; 6,075,181; 6,114,598; 6, 150,584 and 6,162,963 to Kucherlapati et al.

Moreover, alternative transchromosomic animal systems expressing human immunoglobulin genes are available in the art and can be used to raise anti-VSTM5 antibodies according to at least some embodiments of the invention. For example, mice carrying both a human heavy chain transchromosome and a human light chain transchromosome, referred to as “TC mice” can be used; such mice are described in Tomizuka et al. (2000) Proc. Natl. Acad Sci. USA 97:722-727. Furthermore, cows carrying human heavy and light chain transchromosomes have been described in the art (Kuroiwa et al. (2002) Nature Biotechnology 20:889-894) and can be used to raise anti-VSTM5 antibodies according to at least some embodiments of the invention.

Human monoclonal antibodies according to at least some embodiments of the invention can also be prepared using phage display methods for screening libraries of human immunoglobulin genes. Such phage display methods for isolating human antibodies are established in the art. See for example: U.S. Pat. Nos. 5,223,409; 5,403,484; and U.S. Pat. No. 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies according to at least some embodiments of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

Immunization of Human Ig Mice

In some embodiments human Ig mice are used to raise human anti-VSTM5 antibodies according to the invention, e.g., by immunizing such mice with a purified or enriched preparation of VSTM5 antigen and/or recombinant VSTM5, or VSTM5 fusion protein, as described by Lonberg, N. et al. (1994) Nature 368(6474): 856-859; Fishwild, D. et al. (1996) Nature Biotechnology 14: 845-851; and PCT Publication WO 98/24884 and WO 01/14424. Preferably, the mice will be 6-16 weeks of age upon the first infusion. For example, a purified or recombinant preparation (5-50 μg) of VSTM5 antigen can be used to immunize the human Ig mice intraperitoneally.

In general transgenic mice respond when initially immunized intraperitoneally (IP) with antigen in complete Freund's adjuvant, followed by every other week IP immunizations (up to a total of 6) with antigen in incomplete Freund's adjuvant. However, adjuvants other than Freund's are also found to be effective. In addition, whole cells in the absence of adjuvant are found to be highly immunogenic. The immune response can be monitored over the course of the immunization protocol with plasma samples being obtained by retroorbital bleeds. The plasma can be screened by ELISA (as described below), and mice with sufficient titers of anti-VSTM5 human immunoglobulin can be used for fusions. Mice can be boosted intravenously with antigen 3 days before sacrifice and removal of the spleen. It is expected that 2-3 fusions for each immunization may need to be performed. Between 6 and 24 mice are typically immunized for each antigen. Usually both HCo7 and HCo12 strains are used. In addition, both HCo7 and HCo12 transgene can be bred together into a single mouse having two different human heavy chain transgenes (HCo7/HCo 12). Alternatively or additionally, the KM Mouse™ strain can be used.

Generation of Hybridomas Producing Human Monoclonal Antibodies

In certain embodiments, hybridomas producing a human monoclonal anti-VSTM5 antibody according to the invention may be generated using splenocytes and/or lymph node cells from immunized mice can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to one-sixth the number of P3X63-Ag8.653 nonsecreting mouse myeloma cells (ATCC, CRL 1580) with 50% PEG. Cells are plated at approximately 2×10⁻⁵ in flat bottom microtiter plate, followed by a two week incubation in selective medium containing 20% fetal Clone Serum, 18% “653” conditioned media, 5% origen (IGEN), 4 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin and 1×HAT (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again, and if still positive for human IgG, the monoclonal antibodies can be subcloned at least twice by limiting dilution. The stable subclones can then be cultured in vitro to generate small amounts of antibody in tissue culture medium for characterization.

To purify human monoclonal antibodies, selected hybridomas can be grown in two-liter spinner-flasks for monoclonal antibody purification. Supernatants can be filtered and concentrated before affinity chromatography with protein A-Sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD280 using 1.43 extinction coefficient. The monoclonal antibodies can be aliquoted and stored at −80° C.

Generation of Transfectomas Producing Monoclonal Antibodies

In certain embodiments, an anti-VSTM5 antibody according to the invention can be produced in a host cell transfectoma using, for example, a combination of recombinant DNA techniques and gene transfection methods as is well known in the art (e.g., Morrison, S. (1985) Science 229:1202).

For example, to express the antibodies, or antibody fragments thereof, DNAs encoding partial or full-length light and heavy chains, can be obtained by standard molecular biology techniques (e.g., PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the C_{H} segments within the vector and the V_{L} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors according to at least some embodiments of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel (“Gene Expression Technology”, Methods in Enzymology 185, Academic Press, San Diego, Calif. (1990)). It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or β-globin promoter. Still further, regulatory elements composed of sequences from different sources, such as the SR α. promoter system, which contains sequences from the SV40 early promoter and the long terminal repeat of human T cell leukemia virus type 1 (Takebe, Y. et al. (1988) Mol. Cell. Biol. 8:466-472).

In addition to the antibody chain genes and regulatory sequences, the recombinant expression vectors according to at least some embodiments of the invention may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vectors encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies according to at least some embodiments of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies according to at least some embodiments of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. In particular, for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Characterization of Antibody Binding to Antigen

In certain embodiments, the binding specificity of an anti-VSTM5 antibody according to the invention is determined by known antibody binding assay techniques such as ELISA. In an exemplary ELISA, microtiter plates are coated with a purified antigen, herein VSTM5 at 0.25 μg/ml in PBS, and then blocked with 5% bovine serum albumin in PBS. Dilutions of antibody (e.g., dilutions of plasma from—immunized mice) are added to each well and incubated for 1-2 hours at 37° C. The plates are washed with PBS/Tween and then incubated with secondary reagent (e.g., for human antibodies, a goat-anti-human IgG Fc-specific polyclonal reagent) conjugated to alkaline phosphatase for 1 hour at 37° C. After washing, the plates are developed with pNPP substrate (1 mg/ml), and analyzed at OD of 405-650. Preferably, mice which develop the highest titers will be used for fusions.

An ELISA assay as described above can also be used to screen for hybridomas that show positive reactivity with VSTM5 immunogen. Hybridomas that bind with high avidity to VSTM5 are subcloned and further characterized. One clone from each hybridoma, which retains the reactivity of the parent cells (by ELISA), can be chosen for making a 5-10 vial cell bank stored at −140° C., and for antibody purification.

To purify anti-VSTM5 antibodies, selected hybridomas can be grown in two-liter spinner-flasks for monoclonal antibody purification. Supernatants can be filtered and concentrated before affinity chromatography with protein A-Sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD280 using 1.43 extinction coefficient. The monoclonal antibodies can be aliquoted and stored at −80° C. To determine if the selected anti-VSTM5 monoclonal antibodies bind to unique epitopes, each antibody can be biotinylated using commercially available reagents (Pierce, Rockford, Ill.). Competition studies using unlabeled monoclonal antibodies and biotinylated monoclonal antibodies can be performed using VSTM5 coated-ELISA plates as described above. Biotinylated mAb binding can be detected with a strep-avidin-alkaline phosphatase probe.

To determine the isotype of purified antibodies, isotype ELISAs can be performed using reagents specific for antibodies of a particular isotype. For example, to determine the isotype of a human monoclonal antibody, wells of microtiter plates can be coated with 1 μg/ml of anti-human immunoglobulin overnight at 4° C. After blocking with 1% BSA, the plates are reacted with 1 mug/ml or less of test monoclonal antibodies or purified isotype controls, at ambient temperature for one to two hours. The wells can then be reacted with either human IgG1 or human IgM-specific alkaline phosphatase-conjugated probes. Plates are developed and analyzed as described above.

Anti-VSTM5 human IgGs can be further tested for reactivity with VSTM5 antigen, respectively, by Western blotting. Briefly, VSTM5 antigen can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

Alternative Anti-VSTM5 Scaffolds

In certain embodiments, the present invention relates to an antigen-binding construct comprising a protein scaffold which is linked to one or more epitope-binding domains. Such engineered protein scaffolds are usually obtained by designing a random library with mutagenesis focused at a loop region or at an otherwise permissible surface area and by selection of variants against a given target via phage display or related techniques. According to at least some embodiments the invention relates to alternative scaffolds including, but not limited to, anticalins, DARPins, Armadillo repeat proteins, protein A, lipocalins, fibronectin domain, ankyrin consensus repeat domain, thioredoxin, chemically constrained peptides and the like. According to at least some embodiments the invention relates to alternative scaffolds that are used as therapeutic agents for treatment of cancer, autoimmune, infectious diseases, sepsis, or for inhibiting an undesirable immune activation that follows gene therapy, as well as for in vivo diagnostics.

According to at least some embodiments the invention further provides a pharmaceutical composition comprising an antigen-binding construct as described herein a pharmaceutically acceptable carrier.

The term ‘Protein Scaffold’ as used herein includes but is not limited to an immunoglobulin (Ig) scaffold, for example an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions. Such protein scaffolds may comprise antigen-binding sites in addition to the one or more constant regions, for example where the protein scaffold comprises a full IgG. Such protein scaffolds will be capable of being linked to other protein domains, for example protein domains which have antigen-binding sites, for example epitope-binding domains or ScFv domains.

A “domain” is a folded protein structure which has tertiary structure independent of the rest of the protein. Generally, domains are responsible for discrete functional properties of proteins and in many cases may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain.

The phrase “immunoglobulin single variable domain” refers to an antibody variable domain (V_{H}, V_{HH}, V_{L}) that specifically binds an antigen or epitope independently of a different V region or domain. An immunoglobulin single variable domain can be present in a format (e.g., homo- or hetero-multimer) with other, different variable regions or variable domains where the other regions or domains are not required for antigen-binding by the single immunoglobulin variable domain (i.e., where the immunoglobulin single variable domain binds antigen independently of the additional variable domains). A “domain antibody” or “dAb” is the same as an “immunoglobulin single variable domain” which is capable of binding to an antigen as the term is used herein. An immunoglobulin single variable domain may be a human antibody variable domain, but also includes single antibody variable domains from other species such as rodent (for example, as disclosed in WO 00/29004), nurse shark and Camelid V-HH dAbs. Camelid V-HH are immunoglobulin single variable domain polypeptides that are derived from species including camel, llama, alpaca, dromedary, and guanaco, which produce heavy chain antibodies naturally devoid of light chains. Such V-HH domains may be humanized according to standard techniques available in the art, and such domains are still considered to be “domain antibodies” according to the invention. As used herein “V_{H} includes camelid V-HH domains. NARY are another type of immunoglobulin single variable domain which was identified in cartilaginous fish including the nurse shark. These domains are also known as Novel Antigen Receptor variable region (commonly abbreviated to V (NAR) or NARY). See, e.g., Mol. Immunol. 44, 656-665 (2006) and US20050043519A.

The term “epitope-binding domain” refers to a domain that specifically binds an antigen or epitope independently of a different V region or domain, this may be a domain antibody (dAb), for example a human, camelid or shark immunoglobulin single variable domain or it may be a domain which is a derivative of a scaffold selected from the group consisting of CTLA-4 (Evibody®); lipocalin; Protein A derived molecules such as Z-domain of Protein A (Affibody®, SpA), A-domain (Avimer®/Maxibody®); Heat shock proteins such as GroEI and GroES; transferrin (trans-body); ankyrin repeat protein (DARPin®); peptide aptamer; C-type lectin domain (Tetranectin); human &#947;—crystallin and human ubiquitin (affilins); PDZ domains; scorpion toxinkunitz type domains of human protease inhibitors; Armadillo repeat proteins, thioredoxin, and fibronectin (adnectin); which has been subjected to protein engineering in order to obtain binding to a ligand other than the natural ligand.

Loops corresponding to CDRs of antibodies can be substituted with heterologous sequence to confer different binding properties i.e. Evibodies. For further details see Journal of Immunological Methods 248 (1-2), 31-45 (2001) Lipocalins are a family of extracellular proteins which transport small hydrophobic molecules such as steroids, bilins, retinoids and lipids. They have a rigid secondary structure with a number of loops at the open end of the conical structure which can be engineered to bind to different target antigens. Antic alins are between 160-180 amino acids in size, and are derived from lipocalins. For further details see Biochim Biophys Acta 1482: 337-350 (2000), U.S. Pat. No. 7,250,297B1 and US20070224633. An affibody is a scaffold derived from Protein A of Staphylococcus aureus which can be engineered to bind to antigen. The domain consists of a three-helical bundle of approximately 58 amino acids. Libraries have been generated by randomization of surface residues. For further details see Protein Eng. Des. Sel. 17, 455-462 (2004) and EP1641818A1 Avimers are multidomain proteins derived from the A-domain scaffold family. The native domains of approximately 35 amino acids adopt a defined disulphide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007) A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999).

Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two α helices;—β turn. They can be engineered to bind different target antigens by randomizing residues in the first α-helix and a β-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1.

Fibronectin is a scaffold which can be engineered to bind to antigen. Adnectins consists of a backbone of the natural amino acid sequence of the 10th domain of the 15 repeating units of human fibronectin type III (FN3). Three loops at one end of the β-sandwich can be engineered to enable an Adnectin to specifically recognize a therapeutic target of interest. For further details see Protein Eng. Des. Sel. 18, 435-444 (2005), US200801 39791, WO2005056764 and U.S. Pat. No. 6,818,418B1.

Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5:783-797 (2005).

Microbodies are derived from naturally occurring microproteins of 25-50 amino acids in length which contain 3-4 cysteine bridges—examples of microproteins include KalataBI and conotoxin and knottins. The microproteins have a loop which can be engineered to include up to 25 amino acids without affecting the overall fold of the microprotein. For further details of engineered knottin domains, see WO2008098796. Other epitope binding domains include proteins which have been used as a scaffold to engineer different target antigen-binding properties include human β-crystallin and human ubiquitin (affilins), Kunitz type domains of human protease inhibitors, PDZ-domains of the Ras-binding protein AF-6, scorpion toxins (charybdotoxin), C-type lectin domain (tetranectins) are reviewed in Chapter 7—Non-Antibody Scaffolds from Handbook of Therapeutic Antibodies (2007, edited by Stefan Dubel) and Protein Science 15:14-27 (2006). Epitope binding domains of the present invention could be derived from any of these alternative protein domains.

Conjugates or Immunoconjugates

The present invention encompasses conjugates of VSTM5 antigen for use in immune therapy comprising the VSTM5 antigen and soluble portions thereof including the ectodomain or portions or variants thereof. For example the invention encompasses conjugates wherein the ECD of the VSTM5 antigen is attached to an immunoglobulin or fragment thereof. The invention contemplates the use thereof for promoting or inhibiting VSTM5 antigen activities such as immune stimulation and the use thereof in treating transplant, autoimmune, and cancer indications described herein.

In another aspect, the present invention features antibody-drug conjugates (ADCs), used for example for treatment of cancer, consisting of an antibody (or antibody fragment such as a single-chain variable fragment (scFv) linked to a payload drug (often cytotoxic). The antibody causes the ADC to bind to the target cancer cells. Often the ADC is then internalized by the cell and the drug is released into the cell. Because of the targeting, the side effects are lower and give a wider therapeutic window. Hydrophilic linkers (e.g., PEG4Ma1) help prevent the drug being pumped out of resistant cancer cells through MDR (multiple drug resistance) transporters.

In another aspect, the present invention features immunoconjugates comprising an anti-VSTM5 antibody, or a fragment thereof, conjugated to a therapeutic agent, such as a cytotoxin, a drug (e.g., an immunosuppressant) or a radiotoxin. Such conjugates are referred to herein as “immunoconjugates” Immunoconjugates that include one or more cytotoxins are referred to as “immunotoxins.” A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Other preferred examples of therapeutic cytotoxins that can be conjugated to an antibody according to at least some embodiments of the invention include duocarmycins, calicheamicins, maytansines and auristatins, and derivatives thereof. An example of a calicheamicin antibody conjugate is commercially available (Mylotarg™ Wyeth).

Cytotoxins can be conjugated to antibodies according to at least some embodiments of the invention using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D).

For further discussion of types of cytotoxins, linkers and methods for conjugating therapeutic agents to antibodies, see also Saito, G. et al. (2003) Adv. Drug Deliv. Rev. 55:199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3:1089-1091; Senter, P. D. and Springer, C. J. (2001) Adv. Drug Deliv. Rev. 53:247-264.

Antibodies of the present invention also can be conjugated to a radioactive isotope to generate cytotoxic radiopharmaceuticals, also referred to as radioimmunoconjugates. Examples of radioactive isotopes that can be conjugated to antibodies for use diagnostically or therapeutically include, but are not limited to, iodine 131, indium 111, yttrium 90 and lutetium 177. Methods for preparing radioimmunconjugates are established in the art. Radioimmunoconjugates are commercially available, including Zevalin® (BiogenIDEC) and Bexxar®. (Corixa Pharmaceuticals), and similar methods can be used to prepare radioimmunoconjugates using the antibodies according to at least some embodiments of the invention.

The antibody conjugates according to at least some embodiments of the invention can be used to modify a given biological response, and the drug moiety is not to be construed as limited to classical chemical therapeutic agents. For example, the drug moiety may be a protein or polypeptide possessing a desired biological activity. Such proteins may include, for example, an enzymatically active toxin, or active fragment thereof, such as abrin, ricin A, pseudomonas exotoxin, or diphtheria toxin; a protein such as tumor necrosis factor or interferon-γ; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies'84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Bispecific Molecules

According to at least some embodiments the invention encompasses also a multispecific anti-VSTM5 antibody. Multispecific antibodies are monoclonal antibodies that have binding specificities for at least two different sites. In another aspect, the present invention features bispecific molecules comprising an anti-VSTM5 antibody, or a fragment thereof, according to at least some embodiments of the invention. An antibody according to at least some embodiments of the invention, or antigen-binding portions thereof, can be derivatized or linked to another functional molecule, e.g., another peptide or protein (e.g., another antibody or ligand for a receptor) to generate a bispecific molecule that binds to at least two different binding sites or target molecules. The antibody according to at least some embodiments of the invention may in fact be derivatized or linked to more than one other functional molecule to generate multispecific molecules that bind to more than two different binding sites and/or target molecules; such multispecific molecules are also intended to be encompassed by the term “bispecific molecule” as used herein. To create a bispecific molecule according to at least some embodiments of the invention, an antibody can be functionally linked (e.g., by chemical coupling, genetic fusion, noncovalent association or otherwise) to one or more other binding molecules, such as another antibody, antibody fragment, peptide or binding mimetic, such that a bispecific molecule results. In certain embodiments, one of the binding specificities of the bispecific antibodies is for VSTM5 and the other is for any other antigen. In certain embodiments, bispecific antibodies may bind to two different epitopes of VSTM5. Bispecific antibodies may also be used to localize cytotoxic agents to cells which express VSTM5. Bispecific antibodies can be prepared as full length antibodies or antibody fragments.

A bispecific antibody according to at least some embodiments of the invention is an antibody which can bind simultaneously to two targets which are of different structure. Bispecific antibodies (bsAb) and bispecific antibody fragments (bsFab) according to at least some embodiments of the invention have at least one arm that specifically binds to a B-cell antigen or epitope and at least one other arm that specifically binds a targetable conjugate.

According to at least some embodiments the invention encompasses also a fusion antibody protein, which is a recombinantly produced antigen-binding molecule in which two or more different single-chain antibody or antibody fragment segments with the same or different specificities are linked. A variety of bispecific fusion antibody proteins can be produced using molecular engineering. In one form, the bispecific fusion antibody protein is monovalent, consisting of, for example, a sent with a single binding site for one antigen and a Fab fragment with a single binding site for a second antigen. In another form, the bispecific fusion antibody protein is divalent, consisting of, for example, an IgG with two binding sites for one antigen and two scFv with two binding sites for a second antigen.

The invention further encompasses also engineered antibodies with three or more functional antigen-binding sites, including “Octopus antibodies” (see, e.g. US 2006/0025576A1), and “Dual Acting FAb” or “DAF” antibodies comprising an antigen-binding site that binds to VSTM5 as well as another, different antigen (see e.g. US 2008/0069820).

Accordingly, the present invention includes bispecific molecules comprising at least one first binding specificity for VSTM5 and a second binding specificity for a second target epitope. According to at least some embodiments of the invention, the second target epitope is an Fc receptor, e.g., human FcγRI (CD64) or a human Fcα receptor (CD89). Therefore, the invention includes bispecific molecules capable of binding both to FcγR, FcαR or FcεR expressing effector cells (e.g., monocytes, macrophages or polymorphonuclear cells (PMNs)), and to target cells expressing VSTM5, respectively. These bispecific molecules target VSTM5 expressing cells to effector cell and trigger Fc receptor-mediated effector cell activities, such as phagocytosis of an VSTM5 expressing cells, antibody dependent cell-mediated cytotoxicity (ADCC), cytokine release, or generation of superoxide anion.

According to at least some embodiments of the invention in which the bispecific molecule is multispecific, the molecule can further include a third binding specificity, in addition to an anti-Fc binding specificity. In one embodiment, the third binding specificity is an anti-enhancement factor (EF) portion, e.g., a molecule which binds to a surface protein involved in cytotoxic activity and thereby increases the immune response against the target cell.

The “anti-enhancement factor portion” can be an antibody, functional antibody fragment or a ligand that binds to a given molecule, e.g., an antigen or a receptor, and thereby results in an enhancement of the effect of the binding determinants for the Fc receptor or target cell antigen. The “anti-enhancement factor portion” can bind an Fc receptor or a target cell antigen. Alternatively, the anti-enhancement factor portion can bind to an entity that is different from the entity to which the first and second binding specificities bind. For example, the anti-enhancement factor portion can bind a cytotoxic T-cell (e.g., via CD2, CD3, CD8, CD28, CD4, CD40, ICAM-1 or other immune cell that results in an increased immune response against the target cell).

According to at least some embodiments of the invention, the bispecific molecules comprise as a binding specificity at least one antibody, or an antibody fragment thereof, including, e.g., an Fab, Fab′, F(ab′)2, Fv, or a single chain Fv. The antibody may also be a light chain or heavy chain dimer, or any minimal fragment thereof such as a Fv or a single chain construct as described in Ladner et al. U.S. Pat. No. 4,946,778, the contents of which are expressly incorporated by reference.

In one embodiment, the binding specificity for an Fcγ receptor is provided by a monoclonal antibody, the binding of which is not blocked by human immunoglobulin G (IgG). As used herein, the term “IgG receptor” refers to any of the eight γ-chain genes located on chromosome 1. These genes encode a total of twelve transmembrane or soluble receptor isoforms which are grouped into three Fcγ receptor classes: FcγR1 (CD64), FcγRII(CD32), and FcγRIII (CD16). In one preferred embodiment, the Fc γ receptor is a human high affinity FcγRI. The human FcγRI is a 72 kDa molecule, which shows high affinity for monomeric IgG (10⁻⁸-10⁻⁹ M⁻¹).

The production and characterization of certain preferred anti-Fc γ monoclonal antibodies are described by Fanger et al. in PCT Publication WO 88/00052 and in U.S. Pat. No. 4,954,617, the teachings of which are fully incorporated by reference herein. These antibodies bind to an epitope of FcγR1, FcγRII or FcγRIII at a site which is distinct from the Fcγ binding site of the receptor and, thus, their binding is not blocked substantially by physiological levels of IgG. Specific anti-FcγRI antibodies useful in this invention are mAb 22, mAb 32, mAb 44, mAb 62 and mAb 197. The hybridoma producing mAb 32 is available from the American Type Culture Collection, ATCC Accession No. HB9469. In other embodiments, the anti-Fcγ receptor antibody is a humanized form of monoclonal antibody 22 (H22). The production and characterization of the H22 antibody is described in Graziano, R. F. et al. (1995) J. Immunol. 155 (10): 4996-5002 and PCT Publication WO 94/10332. The H22 antibody producing cell line is deposited at the American Type Culture Collection under the designation HAO22CLI and has the accession no. CRL 11177.

In still other preferred embodiments, the binding specificity for an Fc receptor is provided by an antibody that binds to a human IgA receptor, e.g., an Fc-α receptor (Fc αRI(CD89)), the binding of which is preferably not blocked by human immunoglobulin A (IgA). The term “IgA receptor” is intended to include the gene product of one α-gene (Fc αRI) located on chromosome 19. This gene is known to encode several alternatively spliced transmembrane isoforms of 55 to 10 kDa.

FcαRI (CD89) is constitutively expressed on monocytes/macrophages, eosinophilic and neutrophilic granulocytes, but not on non-effector cell populations. Fc α RI has medium affinity (Approximately 5×10⁻⁷ M⁻¹) for both IgA1 and IgA2, which is increased upon exposure to cytokines such as G-CSF or GM-CSF (Morton, H. C. et al. (1996) Critical Reviews in Immunology 16:423-440). Four FcαRI-specific monoclonal antibodies, identified as A3, A59, A62 and A77, which bind FcαRI outside the IgA ligand binding domain, have been described (Monteiro, R. C. et al. (1992) J. Immunol. 148:1764).

FcαRI and FcγRI are preferred trigger receptors for use in the bispecific molecules according to at least some embodiments of the invention because they are (1) expressed primarily on immune effector cells, e.g., monocytes, PMNs, macrophages and dendritic cells; (2) expressed at high levels (e.g., 5,000-100,000 per cell); (3) mediators of cytotoxic activities (e.g., ADCC, phagocytosis); (4) mediate enhanced antigen presentation of antigens, including self-antigens, targeted to them.

While human monoclonal antibodies are preferred, other antibodies which can be employed in the bispecific molecules according to at least some embodiments of the invention are murine, chimeric and humanized monoclonal antibodies.

The bispecific molecules of the present invention can be prepared by conjugating the constituent binding specificities, e.g., the anti-FcR and anti-VSTM5 binding specificities, using methods known in the art. For example, the binding specificity of each bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyld-ithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Preferred conjugating agents are SATA and sulfo-SMCC, both available from Pierce Chemical Co. (Rockford, Ill.). When the binding moieties are antibodies, they can be conjugated via sulfhydryl bonding of the C-terminus hinge regions of the two heavy chains. In a particularly preferred embodiment, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific molecule is a mAbXmAb, mAbXFab, FabXF(ab′)2 or ligandXFab fusion protein. A bispecific molecule according to at least some embodiments of the invention can be a single chain molecule comprising one single chain antibody and a binding determinant, or a single chain bispecific molecule comprising two binding determinants. Bispecific molecules may comprise at least two single chain molecules. Methods for preparing bispecific molecules are described for example in U.S. Pat. No. 5,260,203; U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); controlled Fab-arm exchange (see Labrijn et al., PNAS 110(13):5145-50 (2013)); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Binding of the bispecific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. For example, the FcR-antibody complexes can be detected using e.g., an enzyme-linked antibody or antibody fragment which recognizes and specifically binds to the antibody-FcR complexes. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography.

Uses of Antibodies and Pharmaceutical Compositions Thereof

Cancer Immunotherapy

Unlike tumor-targeted therapies, which are aimed at inhibiting molecular pathways that are crucial for tumor growth and development, and/or depleting tumor cells, cancer immunotherapy is aimed to stimulate the patient's own immune system to eliminate cancer cells, providing long-lived tumor destruction. Various approaches can be used in cancer immunotherapy, among them are therapeutic cancer vaccines to induce tumor-specific T cell responses, and immunostimulatory antibodies (i.e. antagonists of inhibitory receptors=immune checkpoints) to remove immunosuppressive pathways.

Clinical responses with targeted therapy or conventional anti-cancer therapies tend to be transient as cancer cells develop resistance, and tumor recurrence takes place. However, the clinical use of cancer immunotherapy in the past few years has shown that this type of therapy can have durable clinical responses, showing dramatic impact on long term survival. However, although responses are long term, only a small number of patients respond (as opposed to conventional or targeted therapy, where a large number of patients respond, but responses are transient).

By the time a tumor is detected clinically, it has already evaded the immune-defense system by acquiring immunoresistant and immunosuppressive properties and creating an immunosuppressive tumor microenvironment through various mechanisms and a variety of immune cells. Thus, in cancer immunotherapy it is becoming increasingly clear that a combination of therapies is be required for clinical efficacy.

Combination approaches are needed and expected to increase the number of patients benefiting from immunotherapy and expand the number and types of cancers that are responsive, expanding the potential cancer indications for checkpoint agents well beyond the initial indications currently showing efficacy of immune checkpoint blockade as monotherapy. The combination of immunomodulatory approaches is meant to maximize the outcomes and overcome the resistance mechanisms of most tumors to a single approach. Thus, tumors traditionally thought of as non-immunogenic can likely become immunogenic and respond to immunotherapy though co-administration of pro-immunogenic therapies designed to increase the patient's anti-tumor immune responses. Potential priming agents are detailed herein below.

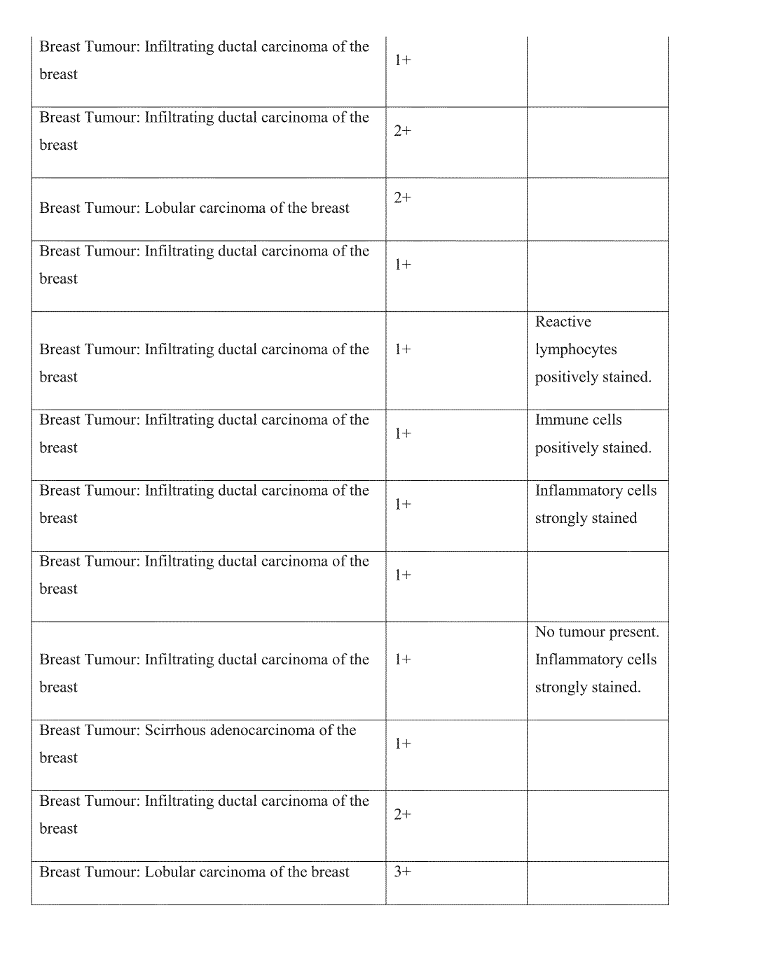

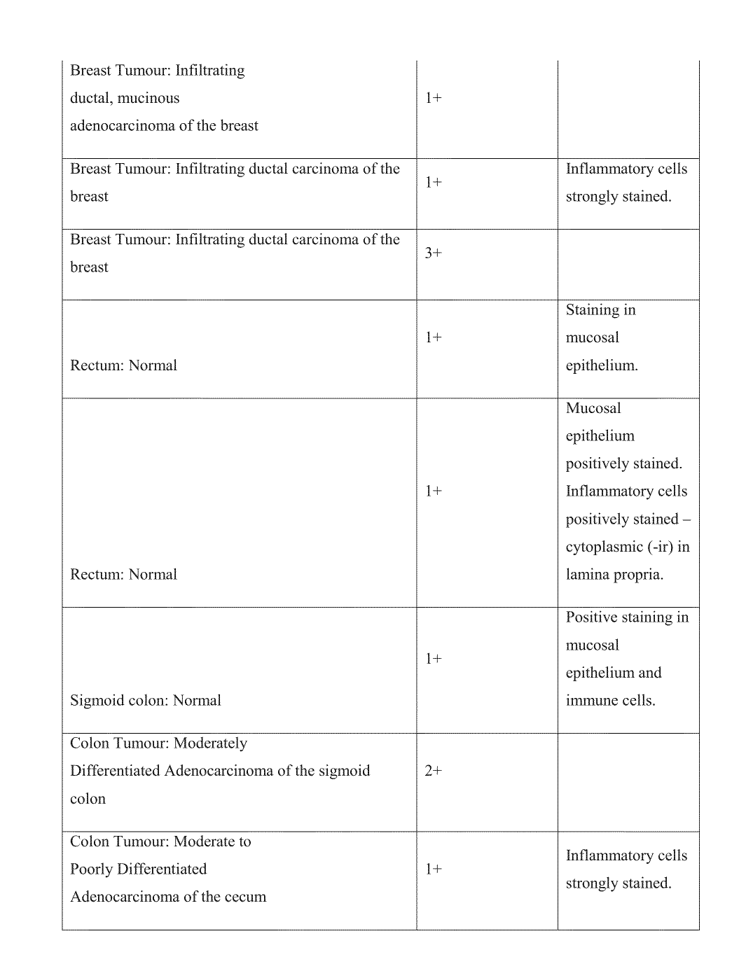

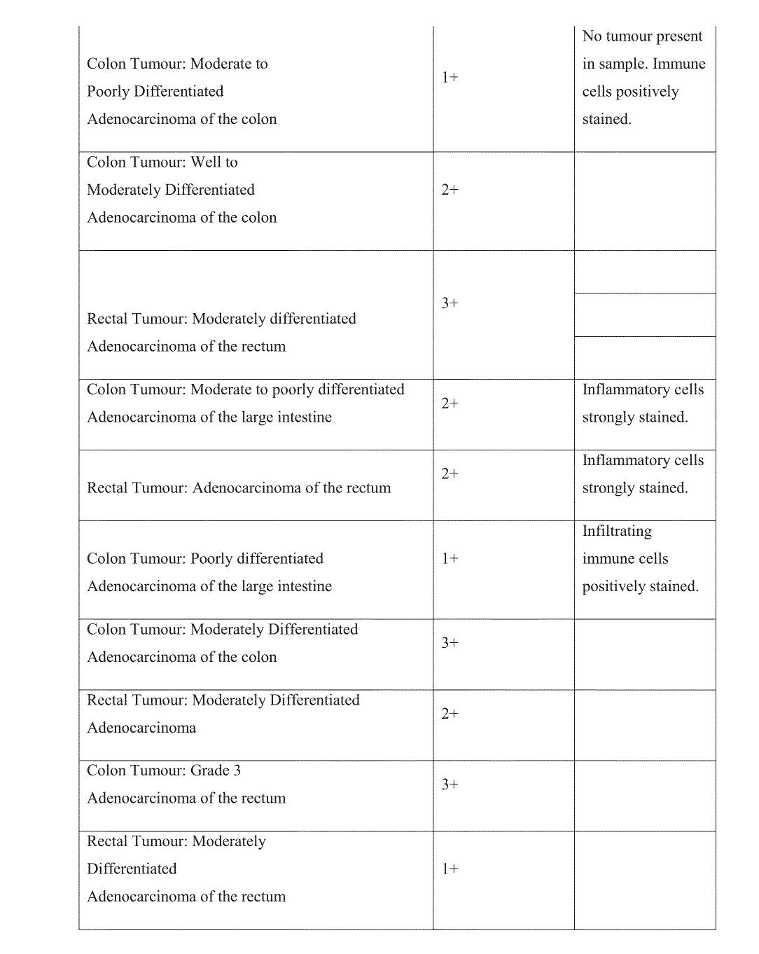

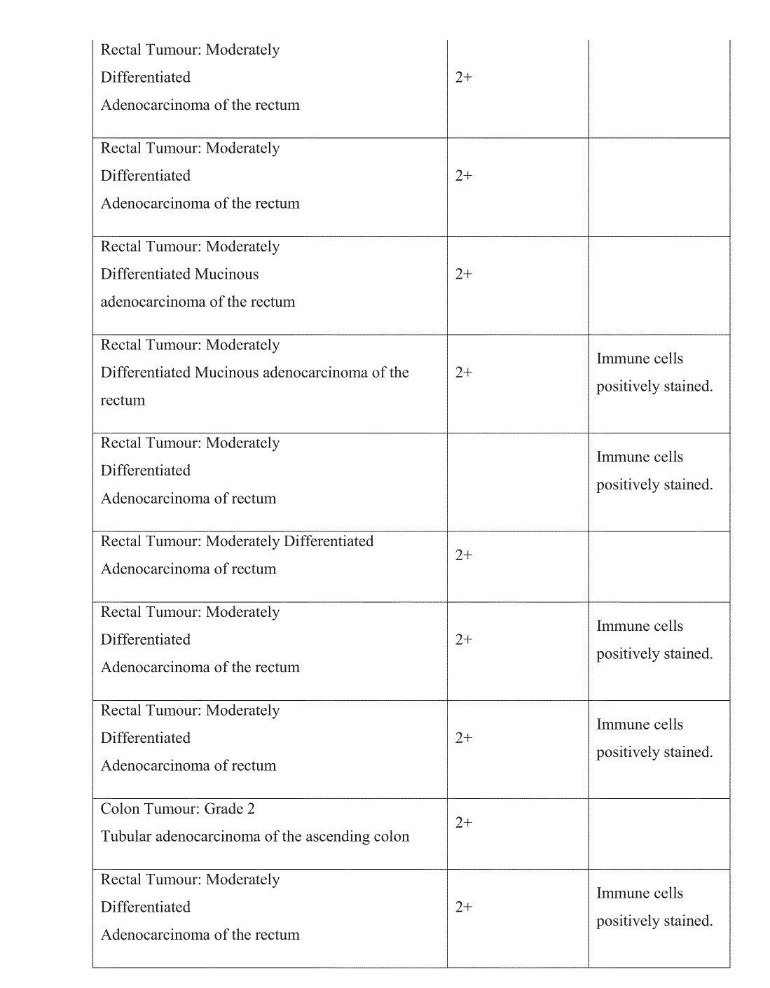

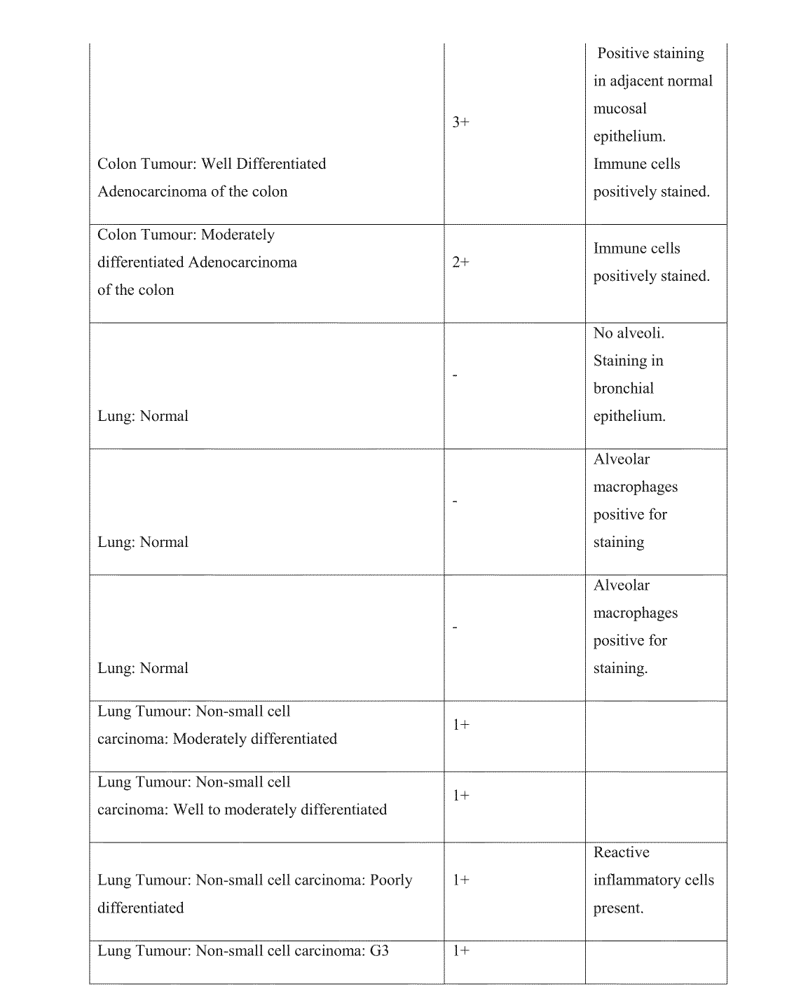

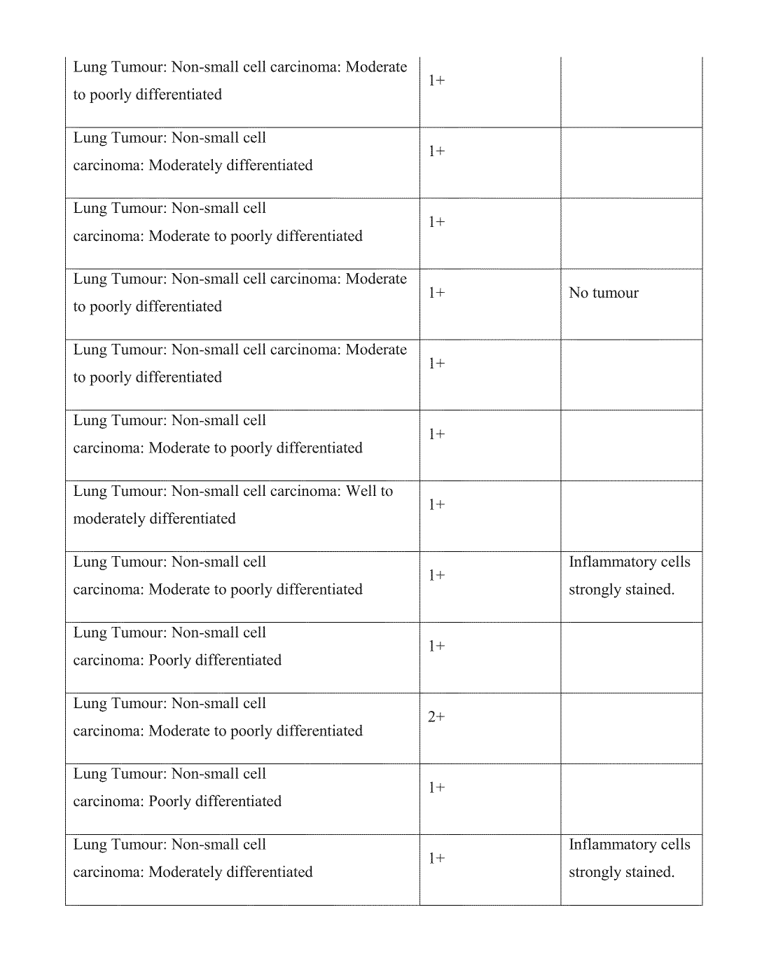

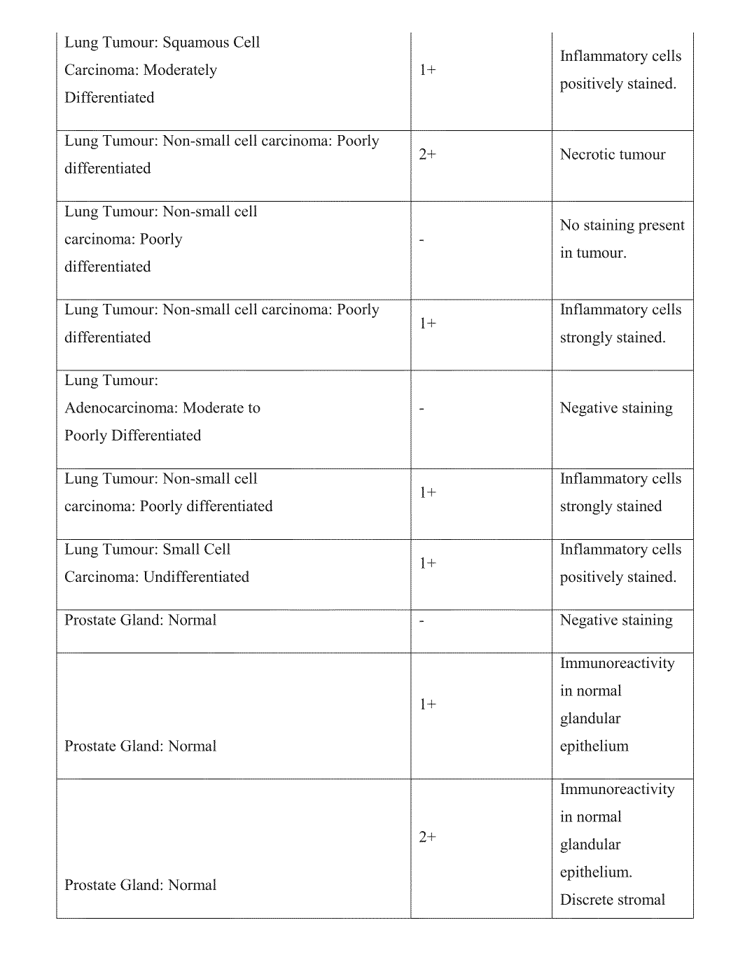

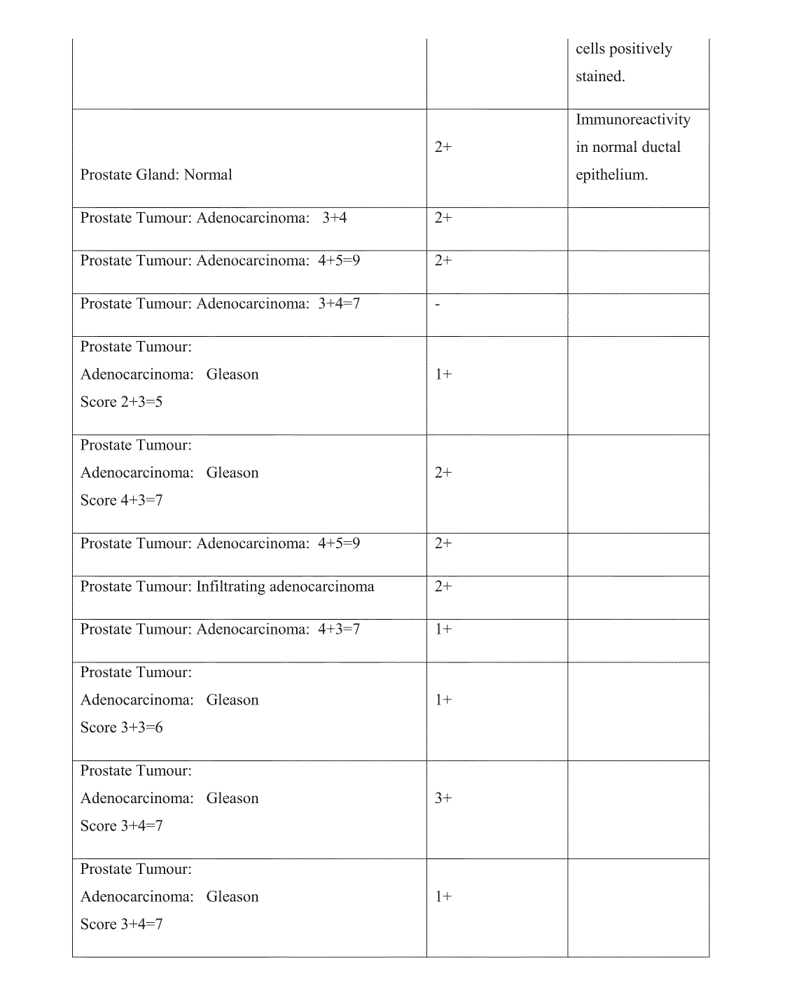

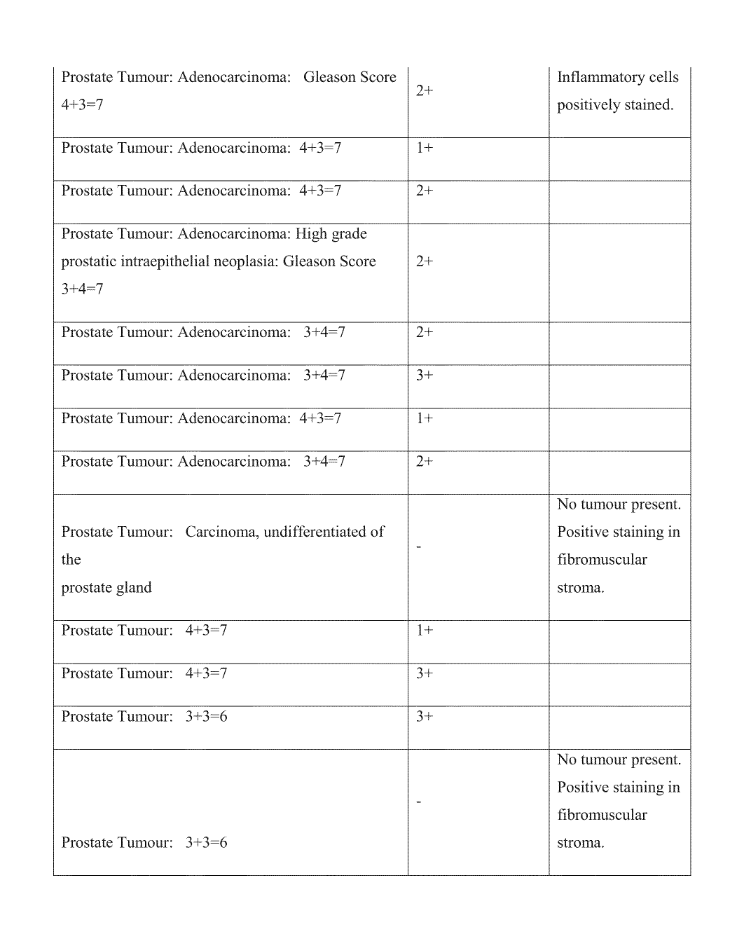

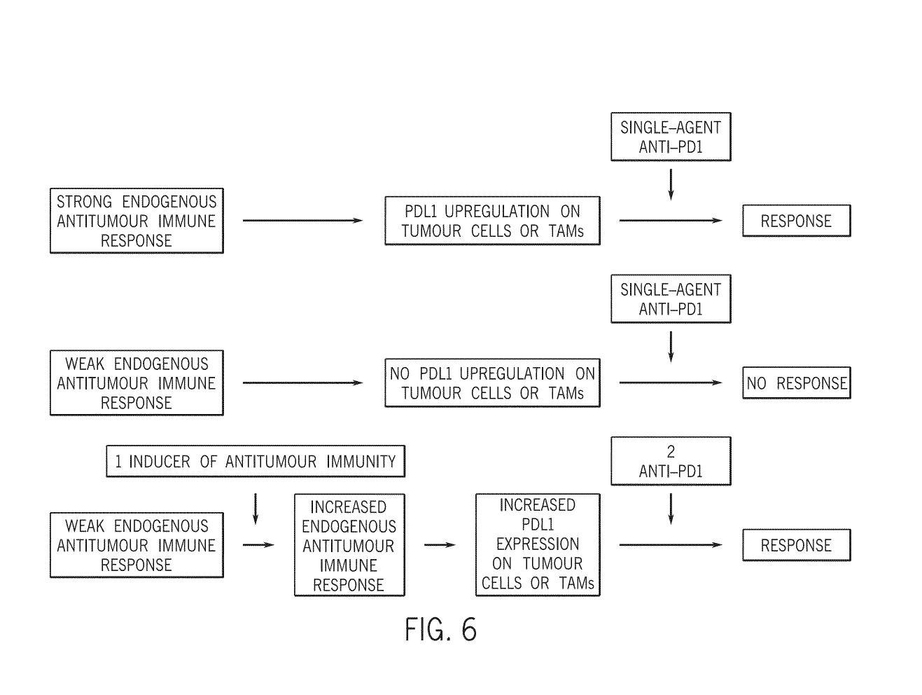

The underlying scientific rationale for the dramatic increased efficacy of combination therapy claims that immune checkpoint blockade as a monotherapy will induce tumor regressions only when there is pre-existing strong anti-tumor immune response to be ‘unleashed’ when the pathway is blocked. However, in most patients and tumor types the endogenous anti-tumor immune responses are weak, and thus the induction of anti-tumor immunity is required for the immune checkpoint blockade to be effective, as shown in the FIG. 6 (which depicts the case of the PDL-1/PD-1 immune checkpoint). As can be appreciated from FIG. 6, the endogenous expression of the immune checkpoint ligand (PDL-1 in this case) is elevated by the induction of anti-tumor immunity, and thus expression in the patient's original tumor is not a prerequisite for the combination therapy to be effective. According to at least some embodiments of the present invention, VSTM5-specific antibodies, antibody fragments, conjugates and compositions comprising same, are used for treatment of all types of cancer in cancer immunotherapy in combination therapy.

The term “treatment” as used herein, refers to both therapeutic treatment and prophylactic or preventative measures, which in this Example relates to treatment of cancer; however, also as described below, uses of antibodies and pharmaceutical compositions are also provided for treatment of infectious disease, sepsis, and/or autoimmune conditions, and/or for inhibiting an undesirable immune activation that follows gene therapy. Those in need of treatment include those already with cancer as well as those in which the cancer is to be prevented. Hence, the mammal to be treated herein may have been diagnosed as having the cancer or may be predisposed or susceptible to the cancer. As used herein the term “treating” refers to preventing, delaying the onset of, curing, reversing, attenuating, alleviating, minimizing, suppressing, halting the deleterious effects or stabilizing of discernible symptoms of the above-described cancerous diseases, disorders or conditions. It also includes managing the cancer as described above. By “manage” it is meant reducing the severity of the disease, reducing the frequency of episodes of the disease, reducing the duration of such episodes, reducing the severity of such episodes, slowing/reducing cancer cell growth or proliferation, slowing progression of at least one symptom, amelioration of at least one measurable physical parameter and the like. For example, immunostimulatory anti-VSTM5 antibodies should promote T cell or NK or cytokine immunity against target cells, e.g., cancer, infected or pathogen cells and thereby treat cancer or infectious diseases by depleting the cells involved in the disease condition. Conversely, immunoinhibitory anti-VSTM5 antibodies should reduce T cell or NK activity and/or or the secretion of proinflammatory cytokines which are involved in the disease pathology of some immune disease such as autoimmune, inflammatory or allergic conditions and thereby treat or ameliorate the disease pathology and tissue destruction that may be associated with such conditions (e.g., joint destruction associated with rheumatoid arthritis conditions).

“Mammal” for purposes of treatment refers to any animal classified as a mammal, including humans, domestic and farm animals, and zoo, sports, or pet animals, such as dogs, horses, cats, cows, etc. Preferably, the mammal is human. Preferably the mammal is a human which is diagnosed with one of the disease, disorder or conditions described hereinabove, or alternatively one who is predisposed to at least one type of cancer.

The term “therapeutically effective amount” refers to an amount of agent according to the present invention that is effective to treat a disease or disorder in a mammal.

The therapeutic agents of the present invention can be provided to the subject alone, or as part of a pharmaceutical composition where they are mixed with a pharmaceutically acceptable carrier.

An anti-VSTM5 antibody, a fragment, a conjugate thereof and/or a pharmaceutical composition comprising same, according to at least some embodiments of the present invention also can be administered in combination therapy, i.e., combined with other potentiating agents and/or other therapies. According to at least some embodiments, the anti VSTM5 antibody could be used in combination with any of the known in the art standard of care cancer treatment (as can be found, for example, in http://www.cancer.gov/cancertopics).

For example, the combination therapy can include an anti VSTM5 antibody, a fragment, a conjugate thereof and/or a pharmaceutical composition comprising same, combined with at least one other therapeutic or immune modulatory agent, other compounds or immunotherapies, or immunostimulatory strategy as described herein.

According to at least some embodiments of the present invention, therapeutic agents that can be used in combination with anti-VSTM5 antibodies are potentiating agents that enhance anti-tumor responses.

Various strategies are available for combining an anti-VSTM5 immunostimulatory antibody with potentiating agents for cancer immunotherapy.

According to at least some embodiments of the present invention, anti-VSTM5 antibody for cancer immunotherapy is used in combination with potentiating agents that are primarily geared to increase endogenous anti-tumor responses, such as Radiotherapy, Cryotherapy, Conventional/classical chemotherapy potentiating anti-tumor immune responses, Targeted therapy potentiating anti-tumor immune responses, Anti-angiogenic therapy, Therapeutic agents targeting immunosuppressive cells such as Tregs and MDSCs, Immunostimulatory antibodies, Cytokine therapy, Therapeutic cancer vaccines, Adoptive cell transfer.

The scientific rationale behind the combined use with some chemotherapy or anti-cancer conventional drugs is that cancer cell death, a consequence of the cytotoxic action of most chemotherapeutic compounds, may result in increased levels of tumor antigen leading to enhanced antigen presentation and stimulation of anti-tumor immune responses (i e immunogenic cell death), resulting in potentiating effects with the anti VSTM5 antibody (Zitvogel et al, 2008, The journal of clinical investigation, vol. 118, pages 1991-2001; Galluzzi et al, 2012, Nature Reviews—Drug discovery, Volume 11, pages 215-233). Other combination therapies that may potentiate anti-tumor responses through tumor cell death are radiotherapy, Cryotherapy, surgery, and hormone deprivation. Each of these cancer therapies creates a source of tumor antigen in the host.

According to at least some embodiments of the present invention, anti-VSTM5 antibody for cancer immunotherapy is used in combination with Bisphosphonates, especially amino-bisphosphonates (ABP), which have shown to have anti-cancer activity. Some of the activities associated with ABPs are on human γδT cells that straddle the interface of innate and adaptive immunity and have potent anti-tumour activity.

Targeted therapies can also stimulate tumor-specific immune response by inducing the immunogenic death of tumor cells or by engaging immune effector mechanisms (Galluzzi et al, 2012, Nature Reviews—Drug discovery, Volume 11, pages 215-233).

According to at least some embodiments of the invention, Targeted therapies used as agents for combination with anti VSTM5 antibodies for treatment of cancer are as described herein.

Other cancer immunotherapies that also increase endogenous anti-tumor responses could also potentiate the effect of the anti VSTM5 antibody by enhancing immune effector mechanisms, such as Adoptive T cell therapy, Therapeutic cancer vaccines, reduced immune suppressive cells and their function, Cytokine therapy, or Immunostimulatory antibodies.

According to at least some embodiments of the present invention, anti-VSTM5 antibody for cancer immunotherapy is used in combination with Therapeutic agents targeting regulatory immunosuppressive cells such as regulatory T cells (Tregs) and myeloid derived suppressor cells (MDSCs). A number of commonly used chemotherapeutics exert non-specific targeting of Tregs and reduce the number or the immunosuppressive capacity of Tregs or MDSCs (Facciabene A. et al 2012 Cancer Res; 72(9) 2162-71; Byrne W L. et al 2011, Cancer Res. 71:691520; Gabrilovich D I. and Nagaraj S, Nature Reviews 2009 Volume 9, pages 162-174). In this regard, metronomic therapy with some chemotherapy drugs results in immunostimulatory rather than immunosuppressive effects, via modulation of regulatory cells. Thus, according to at least some embodiments of the present invention, anti-VSTM5 antibody for cancer immunotherapy is used in combination with drugs selected from but not limited to cyclophosphamide, gemcitabine, mitoxantrone, fludarabine, fludarabine, docetaxel, paclitaxel, thalidomide and thalidomide derivatives.

In addition, according to at least some embodiments of the present invention, anti-VSTM5 antibody for cancer immunotherapy is used in combination with novel Treg-specific targeting agents including: 1) depleting or killing antibodies that directly target Tregs through recognition of Treg cell surface receptors such as anti-CD25 mAbs daclizumab, basiliximab or 2) ligand-directed toxins such as denileukin diftitox (Ontak)—a fusion protein of human IL-2 and diphtheria toxin, or LMB-2—a fusion between an scFv against CD25 and Pseudomonas exotoxin and 3) antibodies targeting Treg cell surface receptors such as CTLA4, PD-1, OX40 and GITR or 4) antibodies, small molecules or fusion proteins targeting other NK receptors such as previously identified.

According to at least some embodiments of the present invention, anti-VSTM5 antibody for cancer immunotherapy is used in combination with any of the options described below for disrupting Treg induction and/or function, including TLR (toll like receptors) agonists; agents that interfere with the adenosinergic pathway, such as ectonucleotidase inhibitors, or inhibitors of the A2A adenosine receptor; TGF-β inhibitors, such as fresolimumab, lerdelimumab, metelimumab, trabedersen, LY2157299, LY210976; blockade of Tregs recruitment to tumor tissues including chemokine receptor inhibitors, such as the CCR4/CCL2/CCL22 pathway.

According to at least some embodiments of the present invention, anti-VSTM5 antibody for cancer immunotherapy is used in combination with any of the options described below for inhibiting the immunosuppressive tumor microenvironment, including inhibitors of cytokines and enzymes which exert immunosuppressive activities, such as IDO (indoleamine-2,3-dioxygenase) inhibitors; inhibitors of anti-inflammatory cytokines which promote an immunosuppressive microenvironment, such as IL-10, IL-35, IL-4 and IL-13; Bevacizumab® which reduces Tregs and favors the differentiation of DCs.

According to at least some embodiments of the present invention, anti-VSTM5 antibody for cancer immunotherapy is used in combination with any of the options described below for targeting MDSCs (myeloid-derived suppressive cells), including promoting their differentiation into mature myeloid cells that do not have suppressive functions by Vitamin D3, or Vitamin A metabolites, such as retinoic acid, all-trans retinoic acid (ATRA); inhibition of MDSCs suppressive activity by COX2 inhibitors, phosphodiesterase 5 inhibitors like sildenafil, ROS inhibitors such as nitroaspirin.

According to at least some embodiments of the present invention, anti-VSTM5 antibody for cancer immunotherapy is used in combination with immunostimulatory antibodies or other agents which potentiate anti-tumor immune responses (Pardoll J Exp Med. 2012; 209(2): 201-209) Immunostimulatory antibodies promote anti-tumor immunity by directly modulating immune functions, i.e. blocking other inhibitory targets or enhancing immunostimulatory proteins. According to at least some embodiments of the present invention, anti-VSTM5 antibody for cancer immunotherapy is used in combination with antagonistic antibodies targeting immune checkpoints including anti-CTLA4 mAbs, such as ipilimumab, tremelimumab; anti-PD-1 such as nivolumab BMS-936558/MDX-1106/ONO-4538, AMP224, CT-011, MK-3475, anti-PDL-1 antagonists such as BMS-936559/MDX-1105, MEDI4736, RG-7446/MPDL3280A; Anti-LAG-3 such as IMP-321), anti-TIM-3, anti-BTLA, anti-B7-H4, anti-B7-H3, Anti-VISTA; Agonistic antibodies targeting immunostimulatory proteins, including anti-CD40 mAbs such as CP-870,893, lucatumumab, dacetuzumab; anti-CD137 mAbs such as BMS-663513 urelumab, PF-05082566; anti-OX40 mAbs, such as anti-OX40; anti-GITR mAbs such as TRX518; anti-CD27 mAbs, such as CDX-1127; and anti-ICOS mAbs.

Cytokines are molecular messengers that allow the cells of the immune system to communicate with one another to generate a coordinated, robust, but self-limited response to a target antigen. Cytokine-based therapies embody a direct attempt to stimulate the patient's own immune system to reject cancer. The growing interest over the past two decades in harnessing the immune system to eradicate cancer has been accompanied by heightened efforts to characterize cytokines and exploit their vast signaling networks to develop cancer treatments. Cytokines directly stimulate immune effector cells and stromal cells at the tumor site and enhance tumor cell recognition by cytotoxic effector cells. Numerous animal tumor model studies have demonstrated that cytokines have broad anti-tumor activity and this has been translated into a number of cytokine-based approaches for cancer therapy (Lee and Margolin 2011, Cancers 3(4):3856-93). A number of cytokines are in preclinical or clinical development as agents potentiating anti-tumor immune responses for cancer immunotherapy, including among others: IL-2, IL-7, IL-12, IL-15, IL-17, IL-18 and IL-21, IL-23, IL-27, GM-CSF, IFNα (interferon α), IFNβ, and IFNγ.

Several cytokines have been approved for therapy of cancer and many more are under development. However, therapeutic efficacy is often hampered by severe side effects and poor pharmacokinetic properties. Thus, in addition to systemic administration of cytokines, a variety of strategies can be employed for the delivery of therapeutic cytokines and their localization to the tumor site, in order to improve their pharmacokinetics, as well as their efficacy and/or toxicity, including antibody-cytokine fusion molecules (immunocytokines), chemical conjugation to polyethylene glycol (PEGylation), transgenic expression of cytokines in autologous whole tumor cells, incorporation of cytokine genes into DNA vaccines, recombinant viral vectors to deliver cytokine genes, etc. In the case of immunocytokines, fusion of cytokines to tumor-specific antibodies or antibody fragments allows for targeted delivery and therefore improved efficacy and pharmacokinetics, and reduced side effects.

According to at least some embodiments of the present invention, anti-VSTM5 antibody for cancer immunotherapy is used in combination with Cytokine therapy, involving the use of cytokines as agents potentiating anti-tumor immune responses, including cytokines such as IL-2, IL-7, IL-12, IL-15, IL-17, IL-18 and IL-21, IL-23, IL-27, GM-CSF, IFNα (interferon α), IFNα-2b, IFNβ, IFNγ, and their different strategies for delivery, as described above.

Cancer vaccines are used to treat existing cancer (therapeutic) or prevent the development of cancer in certain high-risk individuals (prophylactic). Therapeutic cancer vaccines allow for improved priming of T cells and improved antigen presentation, and can be used as therapeutic agents for potentiating anti-tumor immune responses (Mellman I. et al., 2011, Nature, 480:22-29; Schlom J, 2012, J Natl Cancer Inst; 104:599-613).

Several types of therapeutic cancer vaccines are in preclinical and clinical development. These include for example:

1) Whole tumor cell vaccines, in which cancer cells removed during surgery are treated to enhance their immunogenicity, and injected into the patient to induce immune responses against antigens in the tumor cells. The tumor cell vaccine can be autologous, i.e. a patient's own tumor, or allogeneic which typically contain two or three established and characterized human tumor cell lines of a given tumor type, such as the GVAX vaccine platforms.

2) Tumor antigen vaccines, in which a tumor antigen (or a combination of a few tumor antigens), usually proteins or peptides, are administered to boost the immune system (possibly with an adjuvant and/or with immune modulators or attractants of dendritic cells such as GM-CSF). The tumor antigens may be specific for a certain type of cancer, but they are not made for a specific patient.

3) Vector-based tumor antigen vaccines and DNA vaccines can be used as a way to provide a steady supply of antigens to stimulate an anti-tumor immune response. Vectors encoding for tumor antigens are injected into the patient (possibly with proinflammatory or other attractants such as GM-CSF), taken up by cells in vivo to make the specific antigens, which would then provoke the desired immune response. Vectors may be used to deliver more than one tumor antigen at a time, to increase the immune response. In addition, recombinant virus, bacteria or yeast vectors should trigger their own immune responses, which may also enhance the overall immune response.

4) Oncolytic virus vaccines, such as OncoVex/T-VEC, which involves the intratumoral injection of replication-conditional herpes simplex virus which preferentially infects cancer cells. The virus, which is also engineered to express GM-CSF, is able to replicate inside a cancer cell causing its lysis, releasing new viruses and an array of tumor antigens, and secreting GM-CSF in the process. Thus, such oncolytic virus vaccines enhance DCs function in the tumor microenvironment to stimulate anti-tumor immune responses.

5) Dendritic cell vaccines (Palucka and Banchereau, 2102, Nat. Rev. Cancer, 12(4):265-277): Dendritic cells (DCs) phagocytose tumor cells and present tumor antigens to tumor specific T cells. In this approach, DCs are isolated from the cancer patient and primed for presenting tumor-specific T cells. To this end several methods can be used: DCs are loaded with tumor cells or lysates; DCs are loaded with fusion proteins or peptides of tumor antigens; coupling of tumor antigens to DC-targeting mAbs. The DCs are treated in the presence of a stimulating factor (such as GM-CSF), activated and matured ex vivo, and then re-infused back into the patient in order provoke an immune response to the cancer cells. Dendritic cells can also be primed in vivo by injection of patients with irradiated whole tumor cells engineered to secrete stimulating cytokines (such as GM-CSF). Similar approaches can be carried out with monocytes. Sipuleucel-T (Provenge), a therapeutic cancer vaccine which has been approved for treatment of advanced prostate cancer, is an example of a dendritic cell vaccine.

Thus, according to at least some embodiments of the present invention, anti-VSTM5 antibody for cancer immunotherapy is used in combination with Therapeutic cancer vaccines. Non limiting examples of such therapeutic cancer vaccines include Whole tumor cell vaccines, Tumor antigen vaccines, Vector-based vaccines, Oncolytic virus vaccines, Dendritic-cell vaccines, as described above.

One approach to cancer immunotherapy is based on adoptive T cell therapy or adoptive cell transfer (ACT), which involves the ex vivo identification and expansion of autologous naturally occurring tumor specific T cells, which are then adoptively transferred back into the cancer patient (Restifo et al, 2013, Cancer Immunol. Immunother. 62(4):727-36 (2013) Epub Dec. 4 2012). Cells that are infused back into a patient after ex vivo expansion can traffic to the tumor and mediate its destruction. Prior to this adoptive transfer, hosts can be immunodepleted by irradiation and/or chemotherapy. The combination of lymphodepletion, adoptive cell transfer, and a T cell growth factor (such as IL-2), can lead to prolonged tumor eradication in tumor patients. A more novel approach involves the ex vivo genetic modification of normal peripheral blood T cells to confer specificity for tumor-associated antigens. For example, clones of TCRs of T cells with particularly good anti-tumor responses can be inserted into viral expression vectors and used to infect autologous T cells from the patient to be treated. Another option is the use of chimeric antigen receptors (CARs) which are essentially a chimeric immunoglobulin-TCR molecule, also known as a T-body. CARs have antibody-like specificities and recognize MHC-nonrestricted structures on the surface of target cells (the extracellular target-binding module), grafted onto the TCR intracellular domains capable of activating T cells (Restifo et al Cancer Immunol. Immunother. 62(4):727-36 (2013) Epub Dec. 4 2012; and Shi et al, Nature 493:111-115 2013.

According to at least some embodiments of the present invention, anti-VSTM5 antibody for cancer immunotherapy is used in combination with Adoptive cell transfer to potentiate anti-tumor immune responses, including genetically modified T cells, as described above.

The VSTM5 specific antibodies, and/or alternative scaffolds and/or multispecific and bispecific molecules and immunoconjugates, compositions comprising same according to at least some embodiments of the present invention can be co-administered together with one or more other therapeutic agents, which acts in conjunction with or synergistically with the composition according to at least some embodiments of the present invention to treat or prevent the cancer. The VSTM5 related therapeutic agents and the one or more other therapeutic agents can be administered in either order or simultaneously. The other therapeutic agents are for example, a cytotoxic agent, a radiotoxic agent or an immunosuppressive agent. The composition can be linked to the agent (as an immunocomplex) or can be administered separately from the agent. In the latter case (separate administration), the composition can be administered before, after or concurrently with the agent or can be co-administered with other known therapies, e.g., an anti-cancer therapy, e.g., radiation. Such therapeutic agents include, among others, anti-neoplastic agents such as doxorubicin (Adriamycin), cisplatin bleomycin sulfate, carmustine, chlorambucil, and cyclophosphamide hydroxyurea which, by themselves, are only effective at levels which are toxic or subtoxic to a patient. Cisplatin is intravenously administered as a 100 mg/dose once every four weeks and Adriamycin is intravenously administered as a 60-75 mg/ml dose once every 21 days. Co-administration of the human anti-VSTM5 antibodies, or antigen-binding fragments and/or alternative scaffolds thereof, according to at least some embodiments of the present invention with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells which would render them unreactive with the antibody. In other embodiments, the subject can be additionally treated with an agent that modulates, e.g., enhances or inhibits, the expression or activity of Fcγ or Fcγ receptors by, for example, treating the subject with a cytokine. Preferred cytokines for administration during treatment with the multispecific molecule include of granulocyte colony-stimulating factor (G-CSF), granulocyte-macrophage colony-stimulating factor (GM-CSF), interferon-γ (IFN-γ), and tumor necrosis factor (TNFα or TNFβ).

Target-specific effector cells, e.g., effector cells linked to compositions (e.g., human antibodies, multispecific and bispecific molecules) according to at least some embodiments of the present invention can also be used as therapeutic agents. Effector cells for targeting can be human leukocytes such as macrophages, neutrophils or monocytes. Other cells include eosinophils, natural killer cells and other IgG- or IgA-receptor bearing cells. If desired, effector cells can be obtained from the subject to be treated. The target-specific effector cells can be administered as a suspension of cells in a physiologically acceptable solution. The number of cells administered can be in the order of 10 to 10⁻⁹ but will vary depending on the therapeutic purpose. In general, the amount will be sufficient to obtain localization at the target cell, e.g., a tumor cell expressing VSTM5 proteins, and to effect cell killing e.g., by, e.g., phagocytosis. Routes of administration can also vary.

Therapy with target-specific effector cells can be performed in conjunction with other techniques for removal of targeted cells. For example, anti-tumor therapy using the compositions (e.g., human antibodies, multispecific and bispecific molecules) according to at least some embodiments of the present invention and/or effector cells armed with these compositions can be used in conjunction with chemotherapy. Additionally, combination immunotherapy may be used to direct two distinct cytotoxic effector populations toward tumor cell rejection. For example, anti-VSTM5 antibodies linked to anti-Fc-γ RI or anti-CD3 may be used in conjunction with IgG- or IgA-receptor specific binding agents.

Bispecific and multispecific molecules according to at least some embodiments of the present invention can also be used to modulate FcγR or FcγR levels on effector cells, such as by capping and elimination of receptors on the cell surface. Mixtures of anti-Fc receptors can also be used for this purpose.

The therapeutic compositions (e.g., human antibodies, alternative scaffolds multispecific and bispecific molecules and immunoconjugates) according to at least some embodiments of the present invention which have complement binding sites, such as portions from IgG1, -2, or -3 or IgM which bind complement, can also be used in the presence of complement. In one embodiment, ex vivo treatment of a population of cells comprising target cells with a binding agent according to at least some embodiments of the present invention and appropriate effector cells can be supplemented by the addition of complement or serum containing complement. Phagocytosis of target cells coated with a binding agent according to at least some embodiments of the present invention can be improved by binding of complement proteins. In another embodiment target cells coated with the compositions (e.g., human antibodies, multispecific and bispecific molecules) according to at least some embodiments of the present invention can also be lysed by complement. In yet another embodiment, the compositions according to at least some embodiments of the present invention do not activate complement.

The therapeutic compositions (e.g., human antibodies, alternative scaffolds multispecific and bispecific molecules and immunoconjugates) according to at least some embodiments of the present invention can also be administered together with complement. Thus, according to at least some embodiments of the present invention there are compositions, comprising human antibodies, multispecific or bispecific molecules and serum or complement. These compositions are advantageous in that the complement is located in close proximity to the human antibodies, multispecific or bispecific molecules. Alternatively, the human antibodies, multispecific or bispecific molecules according to at least some embodiments of the present invention and the complement or serum can be administered separately.

A “therapeutically effective dosage” of an anti-VSTM5 antibody according to at least some embodiments of the present invention preferably results in a decrease in severity of disease symptoms, an increase in frequency and duration of disease symptom-free periods, an increase in lifespan, disease remission, or a prevention or reduction of impairment or disability due to the disease affliction. For example, for the treatment of VSTM5 positive tumors, a “therapeutically effective dosage” preferably inhibits cell growth or tumor growth by at least about 20%, more preferably by at least about 40%, even more preferably by at least about 60%, and still more preferably by at least about 80% relative to untreated subjects. The ability of a compound to inhibit tumor growth can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject.

One of ordinary skill in the art would be able to determine a therapeutically effective amount based on such factors as the subject's size, the severity of the subject's symptoms, and the particular composition or route of administration selected.

The anti-VSTM5 antibodies, according to at least some embodiments of the present invention, can be used as neutralizing antibodies. A Neutralizing antibody (Nabs), is an antibody that is capable of binding and neutralizing or inhibiting a specific antigen thereby inhibiting its biological effect, for example by blocking the receptors on the cell or the virus, inhibiting the binding of the virus to the host cell. NAbs will partially or completely abrogate the biological action of an agent by either blocking an important surface molecule needed for its activity or by interfering with the binding of the agent to its receptor on a target cell.

As used herein “therapeutic agent” is any one of the monoclonal and/or polyclonal antibodies, and/or antigen-binding fragments, and/or conjugates containing same, and/or alternative scaffolds, thereof comprising an antigen-binding site that binds specifically to any one of the VSTM5 polypeptides or an epitope thereof, adopted for treatment of cancer, as recited herein.

According to an additional aspect of the present invention the therapeutic agents can be used to prevent pathologic inhibition of T cell activity, such as that directed against cancer cells.

According to an additional aspect of the present invention the therapeutic agents can be used to inhibit T cell activation, as can be manifested for example by T cell proliferation and cytokine secretion.

Thus, according to an additional aspect of the present invention there is provided a method of treating cancer as recited herein, and/or for promoting immune stimulation mediated by the VSTM5 polypeptide in a subject by administering to a subject in need thereof an effective amount of any one of the therapeutic agents and/or a pharmaceutical composition comprising any of the therapeutic agents and further comprising a pharmaceutically acceptable diluent or carrier.

A therapeutic agent or pharmaceutical composition according to at least some embodiments of the present invention may also be administered in conjunction with other compounds or immunotherapies. For example, the combination therapy can include a compound of the present invention combined with at least one other therapeutic or immune modulatory agent, or immunostimulatory strategy, including, but not limited to, tumor vaccines, adoptive T cell therapy, Treg depletion, antibodies (e.g. bevacizumab, Erbitux), peptides, pepti-bodies, small molecules, chemotherapeutic agents such as cytotoxic and cytostatic agents (e.g. paclitaxel, cisplatin, vinorelbine, docetaxel, gemcitabine, temozolomide, irinotecan, SFU, carboplatin), immunological modifiers such as interferons and interleukins, immunostimulatory antibodies, growth hormones or other cytokines, folic acid, vitamins, minerals, aromatase inhibitors, RNAi, Histone Deacetylase Inhibitors, proteasome inhibitors, and so forth.

According to at least some embodiments, immune cells, preferably T cells, can be contacted in vivo or ex vivo with the therapeutic agents to modulate immune responses. The T cells contacted with the therapeutic agents can be any cell which expresses the T cell receptor, including α/β and γ/δ T cell receptors. T-cells include all cells which express CD3, including T-cell subsets which also express CD4 and CDS. T-cells include both naive and memory cells and effector cells such as CTL. T-cells also include cells such as Th1, Tc1, Th2, Tc2, Th3, Th17, Th22, Treg, and Tr1 cells. T-cells also include NKT-cells and similar unique classes of the T-cell lineage.

VSTM5 blockade may also be combined with standard cancer treatments. VSTM5 blockade may be effectively combined with chemotherapeutic regimes. In these instances, it may be possible to reduce the dose of chemotherapeutic reagent administered. An example of such a combination is an anti-VSTM5 antibody in combination with Temsirolimus for the treatment of late stage renal cell cancer. Another example of such a combination is an anti-VSTM5 antibody in combination with interleukin-2 (IL-2) for the treatment of late stage renal cell cancer as well as combination with Ipilimumab or BMS-936558. The scientific rationale behind the combined use of VSTM5 blockade and chemotherapy is that cell death, that is a consequence of the cytotoxic action of most chemotherapeutic compounds, should result in increased levels of tumor antigen in the antigen presentation pathway. Other combination therapies that may result in synergy with VSTM5 blockade through cell death are radiotherapy, cryotherapy, surgery, and hormone deprivation. Other additional combination therapies with additional immunomodulatory molecules will synergistically contribute to the stimulation of the immune system to eradicate the cancer. Each of these protocols creates a source of tumor antigen in the host. Angiogenesis inhibitors may also be combined with VSTM5 blockade Inhibition of angiogenesis leads to tumor cell death which may feed tumor antigen into host antigen presentation pathways.

VSTM5 blocking antibodies can also be used in combination with bispecific antibodies that target Fcα or Fcγ receptor-expressing effectors cells to tumor cells (see, e.g., U.S. Pat. Nos. 5,922,845 and 5,837,243). Bispecific antibodies can be used to target two separate antigens. For example anti-Fc receptor/anti-tumor antigen (e.g., Her-2/neu) bispecific antibodies have been used to target macrophages to sites of tumor. This targeting may more effectively activate tumor specific responses. The T cell arm of these responses would be augmented by the use of VSTM5 blockade. Alternatively, antigen may be delivered directly to DCs by the use of bispecific antibodies which bind to tumor antigen and a dendritic cell specific cell surface marker.

Tumors evade host immune surveillance by a large variety of mechanisms. Many of these mechanisms may be overcome by the inactivation of proteins which are expressed by the tumors and which are immunosuppressive. These include among others TGF-β (Kehrl, J. et al. (1986) J. Exp. Med. 163: 1037-1050), IL-10 (Howard, M. & O'Garra, A. (1992) Immunology Today 13: 198-200), and Fas ligand (Hahne, M. et al. (1996) Science 274: 1363-1365). Antibodies to each of these entities may be used in combination with anti-VSTM5 to counteract the effects of the immunosuppressive agent and favor tumor immune responses by the host.

Other antibodies which may be used to activate host immune responsiveness can be used in combination with anti-VSTM5. These include molecules on the surface of dendritic cells which activate DC function and antigen presentation. Anti-CD40 antibodies are able to substitute effectively for T cell helper activity (Ridge, J. et al. (1998) Nature 393: 474-478) and can be used in conjunction with VSTM5 antibodies (Ito, N. et al. (2000) Immunobiology 201 (5) 527-40). Activating antibodies to T cell costimulatory molecules such as OX-40 (Weinberg, A. et al. (2000) Immunol 164: 2160-2169), 4-1BB (Melero, I. et al. (1997) Nature Medicine 3: 682-685 (1997), and ICOS (Hutloff, A. et al. (1999) Nature 397: 262-266) as well as antibodies which block the activity of negative costimulatory molecules such as CTLA-4 (e.g., U.S. Pat. No. 5,811,097, implimumab) or BTLA (Watanabe, N. et al. (2003) Nat Immunol 4:670-9), B7-H4 (Sica, G L et al. (2003) Immunity 18:849-61) PD-1 (may also provide for increased levels of T cell activation.

Bone marrow transplantation is currently being used to treat a variety of tumors of hematopoietic origin. While graft versus host disease is a consequence of this treatment, therapeutic benefit may be obtained from graft vs. tumor responses. VSTM5 blockade can be used to increase the effectiveness of the donor engrafted tumor specific T cells.

There are also several experimental treatment protocols that involve ex vivo activation and expansion of antigen specific T cells and adoptive transfer of these cells into recipients in order to antigen-specific T cells against tumor (Greenberg, R. & Riddell, S. (1999) Science 285: 546-51). These methods may also be used to activate T cell responses to infectious agents such as CMV. Ex vivo activation in the presence of anti-VSTM5 antibodies may be expected to increase the frequency and activity of the adoptively transferred T cells.

Optionally, antibodies to VSTM5 can be combined with an immunogenic agent, such as cancerous cells, purified tumor antigens (including recombinant proteins, peptides, and carbohydrate molecules), cells, and cells transfected with genes encoding immune stimulating cytokines (He et al (2004) J. Immunol. 173:4919-28). Non-limiting examples of tumor vaccines that can be used include peptides of MUC1 for treatment of colon cancer, peptides of MUC-1/CEA/TRICOM for the treatment of ovary cancer, or tumor cells transfected to express the cytokine GM-CSF (discussed further below).

In humans, some tumors have been shown to be immunogenic such as RCC. It is anticipated that by raising the threshold of T cell activation by VSTM5 blockade, we may expect to activate tumor responses in the host.

VSTM5 blockade is likely to be most effective when combined with a vaccination protocol. Many experimental strategies for vaccination against tumors have been devised (see Rosenberg, S., 2000, Development of Cancer Vaccines, ASCO Educational Book Spring: 60-62; Logothetis, C., 2000, ASCO Educational Book Spring: 300-302; Khayat, D. 2000, ASCO Educational Book Spring: 414-428; Foon, K. 2000, ASCO Educational Book Spring: 730-738; see also Restifo, N. and Sznol, M., Cancer Vaccines, Ch. 61, pp. 3023-3043 in DeVita, V. et al. (eds.), 1997, Cancer: Principles and Practice of Oncology. Fifth Edition). In one of these strategies, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination (Dranoff et al. (1993) Proc. Natl. Acad. Sci U.S.A. 90: 3539-43).

The study of gene expression and large scale gene expression patterns in various tumors has led to the definition of so-called tumor specific antigens (Rosenberg, S A (1999) Immunity 10: 281-7). In many cases, these tumor specific antigens are differentiation antigens expressed in the tumors and in the cell from which the tumor arose, for example melanocyte antigens gp100, MAGE antigens, and Trp-2. More importantly, many of these antigens can be shown to be the targets of tumor specific T cells found in the host. VSTM5 blockade may be used in conjunction with a collection of recombinant proteins and/or peptides expressed in a tumor in order to generate an immune response to these proteins. These proteins are normally viewed by the immune system as self-antigens and are therefore tolerant to them. The tumor antigen may also include the protein telomerase, which is required for the synthesis of telomeres of chromosomes and which is expressed in more than 85% of human cancers and in only a limited number of somatic tissues (Kim, N et al. (1994) Science 266: 2011-2013). (These somatic tissues may be protected from immune attack by various means). Tumor antigen may also be “neo-antigens” expressed in cancer cells because of somatic mutations that alter protein sequence or create fusion proteins between two unrelated sequences (i.e. bcr-ab1 in the Philadelphia chromosome), or idiotype from B cell tumors.

Other tumor vaccines may include the proteins from viruses implicated in human cancers such a Human Papilloma Viruses (HPV), Hepatitis Viruses (HBV and HCV) and Kaposi's Herpes Sarcoma Virus (KHSV). Another form of tumor specific antigen which may be used in conjunction with VSTM5 blockade is purified heat shock proteins (HSP) isolated from the tumor tissue itself. These heat shock proteins contain fragments of proteins from the tumor cells and these HSPs are highly efficient at delivery to antigen presenting cells for eliciting tumor immunity (Suot, R & Srivastava, P (1995) Science 269:1585-1588; Tamura, Y. et al. (1997) Science 278:117-120).

Dendritic cells (DC) are potent antigen presenting cells that can be used to prime antigen-specific responses. DC's can be produced ex vivo and loaded with various protein and peptide antigens as well as tumor cell extracts (Nestle, F. et al. (1998) Nature Medicine 4: 328-332). DCs may also be transduced by genetic means to express these tumor antigens as well. DCs have also been fused directly to tumor cells for the purposes of immunization (Kugler, A. et al. (2000) Nature Medicine 6:332-336). As a method of vaccination, DC immunization may be effectively combined with VSTM5 blockade to activate more potent anti-tumor responses.

Use of the therapeutic agents according to at least some embodiments of the invention as adjuvant for cancer vaccination:

Immunization against tumor-associated antigens (TAAs) is a promising approach for cancer therapy and prevention, but it faces several challenges and limitations, such as tolerance mechanisms associated with self-antigens expressed by the tumor cells. Costimulatory molecules such as B7.1 (CD80) and B7.2 (CD86) have improved the efficacy of gene-based and cell-based vaccines in animal models and are under investigation as adjuvant in clinical trials. This adjuvant activity can be achieved either by enhancing the costimulatory signal or by blocking inhibitory signal that is transmitted by negative costimulators expressed by tumor cells (Neighbors et al., 2008 J Immunother.; 31(7):644-55).

According to at least some embodiments of the invention, any one of polyclonal or monoclonal antibody and/or antigen-binding fragments and/or conjugates containing same, and/or alternative scaffolds, specific to any one of VSTM5 proteins, can be used as adjuvant for cancer vaccination. According to at least some embodiments, the invention provides methods for improving immunization against TAAs, comprising administering to a patient an effective amount of any one of polyclonal or monoclonal antibody and/or antigen-binding fragments and/or conjugates containing same, and/or alternative scaffolds, specific to any one of VSTM5 proteins.

Use of the Therapeutic Agents According to at Least Some Embodiments of the Invention for Immunoenhancement

Treatment of Cancer

The therapeutic agents provided herein are generally useful in vivo and ex vivo as immune response-stimulating therapeutics. In general, the disclosed therapeutic agent compositions are useful for treating a subject having or being predisposed to any disease or disorder to which the subject's immune system mounts an immune response. The ability of therapeutic agents to modulate VSTM5 immune signals enable a more robust immune response to be possible. The therapeutic agents according to at least some embodiments of the invention are useful to stimulate or enhance immune responses involving immune cells, such as T cells.

The therapeutic agents according to at least some embodiments of the invention are useful for stimulating or enhancing an immune response in a subject with cancer by administering to a subject an amount of a therapeutic agent effective to stimulate T cells in the subject or by stimulating immune cells of the subject ex vivo with an effective amount of an immunostimulatory anti-VSTM5 antibody according to the invention and the re-infusing the immune cells into the subject.

Use of the Therapeutic Agents in Vaccines

The therapeutic agents according to at least some embodiments of the invention, are administered alone or in combination with any other suitable treatment. In one embodiment the therapeutic agents can be administered in conjunction with, or as a component of a vaccine composition as described above. The therapeutic agents according to at least some embodiments of the invention can be administered prior to, concurrently with, or after the administration of a vaccine. In one embodiment the therapeutic agents is administered at the same time as administration of a vaccine.

Use of Anti-VSTM5 Antibodies and Pharmaceutical Compositions for Treatment of Autoimmune Disease

According to at least some embodiments, VSTM5 antibodies, fragments, conjugates thereof and/or a pharmaceutical composition comprising same, as described herein, which function as VSTM5 stimulating therapeutic agents, may optionally be used for treating an immune system related disease.

Optionally, the immune system related condition comprises an immune related condition, autoimmune diseases as recited herein, transplant rejection and graft versus host disease and/or for blocking or promoting immune stimulation mediated by VSTM5, immune related diseases as recited herein and/or for immunotherapy (promoting or inhibiting immune stimulation).

Optionally the immune condition is selected from autoimmune disease, transplant rejection, or graft versus host disease.

Optionally the treatment is combined with another moiety useful for treating immune related condition.

Thus, treatment of multiple sclerosis using the agents according to at least some embodiments of the present invention may be combined with, for example, any known therapeutic agent or method for treating multiple sclerosis, optionally as described herein.

Thus, treatment of rheumatoid arthritis, using the agents according to at least some embodiments of the present invention may be combined with, for example, any known therapeutic agent or method for treating rheumatoid arthritis, optionally as described herein.

Thus, treatment of IBD, using the agents according to at least some embodiments of the present invention may be combined with, for example, any known therapeutic agent or method for treating IBD, optionally as described herein.

Thus, treatment of psoriasis, using the agents according to at least some embodiments of the present invention may be combined with, for example, any known therapeutic agent or method for treating psoriasis, optionally as described herein.

Thus, treatment of type 1 diabetes, using the agents according to at least some embodiments of the present invention may be combined with, for example, any known therapeutic agent or method for treating type 1 diabetes, optionally as described herein.

Thus, treatment of uveitis, using the agents according to at least some embodiments of the present invention may be combined with, for example, any known therapeutic agent or method for treating uveitis, optionally as described herein.

Thus, treatment for Sjögren's syndrome, using the agents according to at least some embodiments of the present invention may be combined with, for example, any known therapeutic agent or method for treating for Sjögren's syndrome, optionally as described herein.

Thus, treatment for systemic lupus erythematosus, using the agents according to at least some embodiments of the present invention may be combined with, for example, any known therapeutic agent or method for treating for systemic lupus erythematosus, optionally as described herein.

In the above-described therapies preferably a subject with one of the afore-mentioned autoimmune or inflammatory conditions will be administered an immunoinhibitory anti-VSTM5 antibody or antigen-binding fragment according to the invention, which antibody mimics or agonizes at least one VSTM5 mediated effect on immunity, e.g., it suppresses cytotoxic T cells, or NK activity and/or the production of proinflammatory cytokines which are involved in the disease pathology, thereby preventing or ameliorating the disease symptoms and potentially resulting in prolonged disease remission, e.g., because of the induction of TRegs which elicit T cell tolerance or prolonged immunosuppression.

The therapeutic agents and/or a pharmaceutical composition comprising same, as recited herein, according to at least some embodiments of the invention, may be administered as the sole active ingredient or together with other drugs in immunomodulating regimens or other anti-inflammatory agents e.g. for the treatment or prevention of allo- or xenograft acute or chronic rejection or inflammatory or autoimmune disorders, or to induce tolerance.

Use of Antibodies and Pharmaceutical Compositions for Treatment of Infectious Disease

According to at least some embodiments, VSTM5 antibodies, fragments, conjugates thereof and/or a pharmaceutical compositions as described herein, which function as VSTM5 blocking therapeutic agents, may optionally be used for treating infectious disease.

Chronic infections are often characterized by varying degrees of functional impairment of virus-specific T-cell responses, and this defect is a principal reason for the inability of the host to eliminate the persisting pathogen. Although functional effector T cells are initially generated during the early stages of infection, they gradually lose function during the course of the chronic infection as a result of persistent exposure to foreign antigen, giving rise to T cell exhaustion. Exhausted T cells express high levels of multiple co-inhibitory receptors such as CTLA-4, PD-1, and LAGS (Crawford et al., Curr Opin Immunol. 2009; 21:179-186; Kaufmann et al., J Immunol 2009; 182:5891-5897, Sharpe et al., Nat Immunol 2007; 8:239-245). PD-1 overexpression by exhausted T cells was observed clinically in patients suffering from chronic viral infections including HIV, HCV and HBV (Crawford et al., Curr Opin Immunol 2009; 21:179-186; Kaufmann et al., J Immunol 2009; 182:5891-5897, Sharpe et al., Nat Immunol 2007; 8:239-245). There has been some investigation into this pathway in additional pathogens, including other viruses, bacteria, and parasites (Hofmeyer et al., J Biomed Biotechnol. Vol 2011, Art. ID 451694, Bhadra et al., Proc Natl. Acad Sci. 2011; 108(22):9196-201). For example, the PD-1 pathway was shown to be involved in controlling bacterial infection using a sepsis model induced by the standard cecal ligation and puncture method. The absence of PD-1 in knockout mice protected from sepsis-induced death in this model (Huang et al., PNAS 2009: 106; 6303-6308).

T cell exhaustion can be reversed by blocking co-inhibitory pathways such as PD-1 or CTLA-4 (Rivas et al., J Immunol. 2009; 183:4284-91; Golden-Mason et al., J Virol. 2009; 83:9122-30; Hofmeyer et al., J Biomed Biotechnol. Vol 2011, Art. ID 451694), thus allowing restoration of anti-viral immune function. The therapeutic potential of co-inhibition blockade for treating viral infection was extensively studied by blocking the PD-1/PD-L1 pathway, which was shown to be efficacious in several animal models of infection including acute and chronic simian immunodeficiency virus (SIV) infection in rhesus macaques (Valu et al., Nature 2009; 458:206-210) and in mouse models of chronic viral infection, such as lymphocytic choriomeningitis virus (LCMV) (Barber et al., Nature. 2006; 439:682-7), and Theiler's murine encephalomyelitis virus (TMEV) model in SJL/J mice (Duncan and Miller PLoS One. 2011; 6:e18548). In these models PD-1/PD-L1 blockade improved anti-viral responses and promoted clearance of the persisting viruses. In addition, PD-1/PD-L1 blockade increased the humoral immunity manifested as elevated production of specific anti-virus antibodies in the plasma, which in combination with the improved cellular responses leads to decrease in plasma viral loads and increased survival.

As used herein the term “infectious disorder and/or disease” and/or “infection”, used interchangeably, includes any disorder, disease and/or condition caused by presence and/or growth of pathogenic biological agent in an individual host organism. As used herein the term “infection” comprises the disorder, disease and/or condition as above, exhibiting clinically evident illness (i.e., characteristic medical signs and/or symptoms of disease) and/or which is asymtomatic for much or all of it course. As used herein the term “infection” also comprises disorder, disease and/or condition caused by persistence of foreign antigen that lead to exhaustion T cell phenotype characterized by impaired functionality which is manifested as reduced proliferation and cytokine production. As used herein the term “infectious disorder and/or disease” and/or “infection”, further includes any of the below listed infectious disorders, diseases and/or conditions, caused by a bacterial infection, viral infection, fungal infection and/or parasite infection.

According to at least some embodiments of the present invention, there is provided use of a combination of the therapeutic agents and/or a pharmaceutical composition comprising same, as recited herein, and a known therapeutic agent effective for treating infection.

The therapeutic agents and/or a pharmaceutical composition comprising same, as recited herein, can be administered in combination with one or more additional therapeutic agents used for treatment of bacterial infections, optionally as described herein.

The therapeutic agents and/or a pharmaceutical composition comprising same, as recited herein, can be administered in combination with one or more additional therapeutic agents used for treatment of viral infections, optionally as described herein.

The therapeutic agents and/or a pharmaceutical composition comprising same, as recited herein, can be administered in combination with one or more additional therapeutic agents used for treatment of fungal infections, optionally as described herein.

In the above-described therapies preferably a subject with one of the afore-mentioned infectious conditions will be administered an immunostimulatory anti-VSTM5 antibody or antigen-binding fragment according to the invention, which antibody antagonizes at least one VSTM5 mediated effect on immunity, e.g., its inhibitory effect on cytotoxic T cells or NK activity and/or its inhibitory effect on the production of proinflammatory cytokines, or inhibits the stimulatory effect of VSTM5 on TRegs thereby prompting the depletion or killing of the infected cells or the pathogen, and potentially resulting in disease remission based on enhanced killing of the pathogen or infected cells by the subject's immune cells.

Use of Antibodies and Pharmaceutical Compositions for Treatment of Sepsis

According to at least some embodiments, VSTM5 antibodies, fragments, conjugates thereof and/or a pharmaceutical compositions as described herein, which function as VSTM5 blocking therapeutic agents, may optionally be used for treating sepsis.

Sepsis is a potentially life-threatening complication of an infection. Sepsis represents a complex clinical syndrome that develops when the initial host response against an infection becomes inappropriately amplified and dysregulated, becoming harmful to the host. The initial hyperinflammatory phase (‘cytokine storm’) in sepsis is followed by a state of immunosuppression (Hotchkiss et al 2013 Lancet Infect. Dis. 13:260-268). This latter phase of impaired immunity, also referred to as ‘immunoparalysis’, is manifested in failure to clear the primary infection, reactivation of viruses such as HSV and cytomegalovirus, and development of new, secondary infections, often with organisms that are not particularly virulent to the immunocompetent patient. The vast majority of septic patients today survive their initial hyperinflammatory insult only to end up in the intensive care unit with sepsis-induced multi-organ dysfunction over the ensuing days to weeks. Sepsis-induced immunosuppression is increasingly recognized as the overriding immune dysfunction in these vulnerable patients. The impaired pathogen clearance after primary infection and/or susceptibility to secondary infections contribute to the high rates of morbidity and mortality associated with sepsis.

Upregulation of inhibitory proteins has lately emerged as one of the critical mechanisms underlying the immunosuppression in sepsis. The PD-1/PDL-1 pathway, for example, appears to be a determining factor of the outcome of sepsis, regulating the delicate balance between effectiveness and damage by the antimicrobial immune response. During sepsis in an experimental model, peritoneal macrophages and blood monocytes markedly increased PD-1 levels, which was associated with the development of cellular dysfunction (Huang et al 2009 PNAS 106:6303-6308). Similarly, in patients with septic shock the expression of PD-1 on peripheral T cells and of PDL-1 on monocytes was dramatically upregulated (Zhang et al 2011 Crit. Care 15:R70). Recent animal studies have shown that blockade of the PD-1/PDL-1 pathway by anti-PD1 or anti-PDL1 antibodies improved survival in sepsis (Brahmamdam et al 2010 J. Leukoc. Biol. 88:233-240; Zhang et al 2010 Critical Care 14:R220; Chang et al 2013 Critical Care 17:R85). Similarly, blockade of CTLA-4 with anti-CTLA4 antibodies improved survival in sepsis (Inoue et al 2011 Shock 36:38-44; Chang et al 2013 Critical Care 17:R85). Taken together, these findings suggest that blockade of inhibitory proteins, including negative costimulatory molecules, is a potential therapeutic approach to prevent the detrimental effects of sepsis (Goyert and Silver, J Leuk. Biol., 88(2): 225-226, 2010).

As used herein, the term “sepsis” or “sepsis related condition” encompasses Sepsis, Severe sepsis, Septic shock, Systemic inflammatory response syndrome (SIRS), Bacteremia, Septicemia, Toxemia, Septic syndrome.

According to at least some embodiments of the present invention, there is provided use of a combination of the therapeutic agents and/or a pharmaceutical composition comprising same, as recited herein, and a known therapeutic agent effective for treating sepsis.

The restoration of the delicate balance that normally exists between the active and suppressor arms of the immune system in sepsis patients may depend on the precise nature of the imbalance, i.e. the pathogenic organism responsible for the infection, its location, the amount of time passed since onset of infection, and other individual parameters. Thus, the correct choice of tools may well depend on the specific immune status or deficit of each individual patient, and may require combination of different drugs.

According to at least some embodiments of the present invention, there is provided use of a combination of the therapeutic agents and/or a pharmaceutical composition comprising same, as recited herein, can be combined with standard of care or novel treatments for sepsis, with therapies that block the cytokine storm in the initial hyperinflammatory phase of sepsis, and/or with therapies that have immunostimulatory effect in order to overcome the sepsis-induced immunosuppression phase.

Combination with standard of care treatments for sepsis, as recommended by the “International Guidelines for Management of Severe Sepsis and Septic Shock” (Dellinger et al 2013 Intensive Care Med 39:165-228), some of which are described below.

Broad spectrum antibiotics having activity against all likely pathogens (bacterial and/or fungal—treatment starts when sepsis is diagnosed, but specific pathogen is not identified)—example Cefotaxime (Claforan®), Ticarcillin and clavulanate (Timentin®), Piperacillin and tazobactam (Zosyn®), Imipenem and cilastatin (Primaxin®), Meropenem (Merrem®), Clindamycin (Cleocin), Metronidazole (Flagyl®), Ceftriaxone (Rocephin®), Ciprofloxacin (Cipro®), Cefepime (Maxipime®), Levofloxacin (Levaquin®), Vancomycin or any combination of the listed drugs.

Vasopressors: example Norepinephrine, Dopamine, Epinephrine, vasopressin

Steroids: example: Hydrocortisone, Dexamethasone, or Fludrocortisone, intravenous or otherwise

Inotropic therapy: example Dobutamine for sepsis patients with myocardial dysfunction

Recombinant human activated protein C (rhAPC), such as drotrecogin alfa (activated) (DrotAA).

β-blockers additionally reduce local and systemic inflammation.

Metabolic interventions such as pyruvate, succinate or high dose insulin substitutions.

Combination with novel potential therapies for sepsis:

Selective inhibitors of sPLA2-IIA (such as LY315920NA/S-5920). Rationale: The Group IIA secretory phospholipase A2 (sPLA2-IIA), released during inflammation, is increased in severe sepsis, and plasma levels are inversely related to survival.

Phospholipid emulsion (such as GR270773). Rationale: Preclinical and ex vivo studies show that lipoproteins bind and neutralize endotoxin, and experimental animal studies demonstrate protection from septic death when lipoproteins are administered. Endotoxin neutralization correlates with the amount of phospholipid in the lipoprotein particles.

anti-TNF-α antibody: Rationale: Tumor necrosis factor-α (TNF-α) induces many of the pathophysiological signs and symptoms observed in sepsis

anti-CD14 antibody (such as IC14). Rationale: Upstream recognition molecules, like CD14, play key roles in the pathogenesis. Bacterial cell wall components bind to CD14 and co-receptors on myeloid cells, resulting in cellular activation and production of proinflammatory mediators. An anti-CD14 monoclonal antibody (IC14) has been shown to decrease lipopolysaccharide-induced responses in animal and human models of endotoxemia.

Inhibitors of Toll-like receptors (TLRs) and their downstream signaling pathways. Rationale: Infecting microbes display highly conserved macromolecules (e.g., lipopolysaccharides, peptidoglycans) on their surface. When these macromolecules are recognized by pattern-recognition receptors (called Toll-like receptors [TLRs]) on the surface of immune cells, the host's immune response is initiated. This may contribute to the excess systemic inflammatory response that characterizes sepsis Inhibition of several TLRs is being evaluated as a potential therapy for sepsis, in particular TLR4, the receptor for Gram-negative bacteria outer membrane lipopolysaccharide or endotoxin. Various drugs targeting TLR4 expression and pathway have a therapeutic potential in sepsis (Wittebole et al 2010 Mediators of Inflammation Vol 10 Article ID 568396). Among these are antibodies targeting TLR4, soluble TLR4, Statins (such as Rosuvastatin®, Simvastatin®), Ketamine, nicotinic analogues, eritoran (E5564), resatorvid (TAK242). In addition, antagonists of other TLRs such as chloroquine, inhibition of TLR-2 with a neutralizing antibody (anti-TLR-2).

Lansoprazole through its action on SOCS1 (suppressor of cytokine secretion)

Talactoferrin or Recombinant Human Lactoferrin. Rationale: Lactoferrin is a glycoprotein with anti-infective and anti-inflammatory properties found in secretions and immune cells. Talactoferrin alfa, a recombinant form of human lactoferrin, has similar properties and plays an important role in maintaining the gastrointestinal mucosal barrier integrity. Talactoferrin showed efficacy in animal models of sepsis, and in clinical trials in patients with severe sepsis (Guntupalli et al Crit Care Med. 2013; 41(3):706-716).

Milk fat globule EGF factor VIII (MFG-E8)—a bridging molecule between apoptotic cells and phagocytes, which promotes phagocytosis of apoptotic cells.

Agonists of the ‘cholinergic anti-inflammatory pathway’, such as nicotine and analogues. Rationale: Stimulating the vagus nerve reduces the production of cytokines, or immune system mediators, and blocks inflammation. This nerve “circuitry”, called the “inflammatory reflex”, is carried out through the specific action of acetylcholine, released from the nerve endings, on the α7 subunit of the nicotinic acetylcholine receptor (α7nAChR) expressed on macrophages, a mechanism termed ‘the cholinergic anti-inflammatory pathway’. Activation of this pathway via vagus nerve stimulation or pharmacologic α7 agonists prevents tissue injury in multiple models of systemic inflammation, shock, and sepsis (Matsuda et al 2012 J Nippon Med Sch. 79:4-18; Huston 2012 Surg. Infect. 13:187-193).

Therapeutic agents targeting the inflammasome pathways. Rationale: The inflammasome pathways greatly contribute to the inflammatory response in sepsis, and critical elements are responsible for driving the transition from localized inflammation to deleterious hyperinflammatory host response (Cinel and Opal 2009 Crit. Care Med. 37:291-304; Matsuda et al 2012 J Nippon Med Sch. 79:4-18).

Stem cell therapy. Rationale: Mesenchymal stem cells (MSCs) exhibit multiple beneficial properties through their capacity to home to injured tissue, activate resident stem cells, secrete paracrine signals to limit systemic and local inflammatory response, beneficially modulate immune cells, promote tissue healing by decreasing apoptosis in threatened tissues and stimulating neoangiogenesis, and exhibit direct antimicrobial activity. These effects are associated with reduced organ dysfunction and improved survival in sepsis animal models, which have provided evidence that MSCs may be useful therapeutic adjuncts (Wannemuehler et al 2012 J. Surg. Res. 173:113-26).

Combination of anti-VSTM5 antibody with other immunomodulatory agents, such as immunostimulatory antibodies, cytokine therapy, immunomodulatory drugs. Such agents bring about increased immune responsiveness, especially in situations in which immune defenses (whether innate and/or adaptive) have been degraded, such as in sepsis-induced hypoinflammatory and immunosuppressive condition. Reversal of sepsis-induced immunoparalysis by therapeutic agents that augments host immunity may reduce the incidence of secondary infections and improve outcome in patients who have documented immune suppression (Hotchkiss et al 2013 Lancet Infect. Dis. 13:260-268; Payen et al 2013 Crit Care. 17:118).

Immunostimulatory antibodies promote immune responses by directly modulating immune functions, i.e. blocking other inhibitory proteins or by enhancing costimulatory proteins. Experimental models of sepsis have shown that immunostimulation by antibody blockade of inhibitory proteins, such as PD-1, PDL-1 or CTLA-4 improved survival in sepsis (Brahmamdam et al 2010 J. Leukoc. Biol. 88:233-240; Zhang et al 2010 Critical Care 14:R220; Chang et al 2013 Critical Care 17:R85; Inoue et al 2011 Shock 36:38-44), pointing to such immunostimulatory agents as potential therapies for preventing the detrimental effects of sepsis-induced immunosuppression (Goyert and Silver J Leuk. Biol. 88(2):225-226, 2010) Immunostimulatory antibodies include: 1) Antagonistic antibodies targeting inhibitory immune checkpoints include anti-CTLA4 mAbs (such as ipilimumab, tremelimumab), Anti-PD-1 (such as nivolumab BMS-936558/MDX-1106/ONO-4538, AMP224, CT-011, lambrozilumab MK-3475), Anti-PDL-1 antagonists (such as BMS-936559/MDX-1105, MEDI4736, RG-7446/MPDL3280A); Anti-LAG-3 such as IMP-321), Anti-TIM-3, Anti-BTLA, Anti-B7-H4, Anti-B7-H3, anti-VISTA. 2) Agonistic antibodies enhancing immunostimulatory proteins include Anti-CD40 mAbs (such as CP-870,893, lucatumumab, dacetuzumab), Anti-CD137 mAbs (such as BMS-663513 urelumab, PF-05082566), Anti-OX40 mAbs (such as Anti-OX40), Anti-GITR mAbs (such as TRX518), Anti-CD27 mAbs (such as CDX-1127), and Anti-ICOS mAbs.

Cytokines which directly stimulate immune effector cells and enhance immune responses can be used in combination with anti-GEN antibody for sepsis therapy: IL-2, IL-7, IL-12, IL-15, IL-17, IL-18 and IL-21, IL-23, IL-27, GM-CSF, IFNα (interferon α), IFNβ, IFNγ. Rationale: Cytokine-based therapies embody a direct attempt to stimulate the patient's own immune system. Experimental models of sepsis have shown administration of cytokines, such as IL-7 and IL-15, promote T cell viability and result in improved survival in sepsis (Unsinger et al 2010 J. Immunol. 184:3768-3779; Inoue et al 2010 J. Immunol. 184:1401-1409). Interferon-γ (IFNγ) reverses sepsis-induced immunoparalysis of monocytes in vitro. An in vivo study showed that IFNγ partially reverses immunoparalysis in vivo in humans. IFNγ and granulocyte-macrophage colony-stimulating factor (GM-CSF) restore immune competence of ex vivo stimulated leukocytes of patients with sepsis (Mouktaroudi et al Crit Care. 2010; 14: P17; Leentjens et al Am J Respir Crit Care Med Vol 186, pp 838-845, 2012).

Immunomodulatory drugs such as thymosin α1. Rationale: Thymosin α1 (Tα1) is a naturally occurring thymic peptide which acts as an endogenous regulator of both the innate and adaptive immune systems. It is used worldwide for treating diseases associated with immune dysfunction including viral infections such as hepatitis B and C, certain cancers, and for vaccine enhancement. Notably, recent development in immunomodulatory research has indicated the beneficial effect of Ta1 treatment in septic patients (Wu et al. Critical Care 2013, 17:R8).

In the above-described sepsis therapies preferably a subject with sepsis or at risk of developing sepsis because of a virulent infection, e.g., one resistant to antibiotics or other drugs, will be administered an immunostimulatory anti-VSTM5 antibody or antigen-binding fragment according to the invention, which antibody antagonizes at least one VSTM5 mediated effect on immunity, e.g., its inhibitory effect on cytotoxic T cells or NK activity and/or its inhibitory effect on the production of proinflammatory cytokines, or inhibits the stimulatory effect of VSTM5 on TRegs thereby promoting the depletion or killing of the infected cells or the pathogen, and potentially resulting in disease remission based on enhanced killing of the pathogen or infected cells by the subject's endogenous immune cells. Because sepsis may rapidly result in organ failure, in this embodiment it may be beneficial to administer anti-VSTM5 antibody fragments such as Fabs rather than intact antibodies as they may reach the site of sepsis and infection quicker than intact antibodies. (In such treatment regimens antibody half-life may be of lesser concern due to the sometimes rapid morbidity of this disease).

Use of Anti-VSTM5 Antibodies and Pharmaceutical Compositions for Reducing the Undesirable Immune Activation that Follows Gene or Cell Therapy or Transplant

As used herein the term “gene therapy” encompasses any type of gene therapy, vector-mediated gene therapy, gene transfer, virus-mediated gene transfer.

According to at least some embodiments of the present invention, VSTM5 antibodies, a fragment, a conjugate thereof and/or a pharmaceutical compositions as described herein, which target VSTM5 and have inhibitory activity on immune responses, could be used as therapeutic agents for reducing the undesirable immune activation that follows gene therapy used for treatment of various genetic diseases. Without wishing to be limited by a single hypothesis, such antibodies have VSTM5-like inhibitory activity on immune responses and/or enhance VSTM5 immune inhibitory activity, optionally by inhibition of pathogenic T cells and/or NK cells.

Gene therapy products for the treatment of genetic diseases are currently in clinical trials. Recent studies document therapeutic success for several genetic diseases using gene therapy vectors. Gene therapy strategies are characterized by 3 critical elements, the gene to be transferred, the target tissue into which the gene will be introduced, and the vector (gene delivery vehicle) used to facilitate entry of the gene to the target tissue. The vast majority of gene therapy clinical trials have exploited viral vectors as very efficient delivery vehicles, including retroviruses, lentiviruses, adenoviruses, adeno-associated viruses, pseudotype viruses and herpes simplex viruses. However, the interactions between the human immune system and all the components of gene therapy vectors seem to represent one of the major limitations to long-lasting therapeutic efficacy. Human studies have shown that the likelihood of a host immune response to the viral vector is high. Such immune responses to the virus or the transgene product itself, resulting in formation of neutralizing antibodies and/or destruction of transduced cells by cytotoxic cells, can greatly interfere with therapeutic efficacy (Seregin and Amalfitano 2010 Viruses 2:2013; Mingozzi and High 2013 Blood 122:23; Masat et al 2013 Discov Med. 15:379). Therefore, developing strategies to circumvent immune responses and facilitate long-term expression of transgenic therapeutic proteins is one of the main challenges for the success of gene therapy in the clinic.

Factors influencing the immune response against transgenic proteins encoded by viral vectors include route of administration, vector dose, immunogenicity of the transgenic protein, inflammatory status of the host and capsid serotype. These factors are thought to influence immunogenicity by triggering innate immunity, cytokine production, APC maturation, antigen presentation and, ultimately, priming of naïve T lymphocytes to functional effectors (Mingozzi and High 2013 Blood 122:23). Therefore, the idea to dampen immune activation by interfering with these very mechanisms has logically emerged with the aim to induce a short-term immunosuppression, avoid the early immune priming that follows vector administration and promote long-term tolerance.

As a strategy to inhibit the undesirable immune activation that follows gene therapy, particularly after multiple injections, immunomodulation treatment by targeting of two non-redundant checkpoints of the immune response at the time of vector delivery was tested in animal models. Studies of vector-mediated immune responses upon adenoviral vector instilled into the lung in mice or monkeys showed that transient treatment with an anti-CD40L antibody lead to suppression of adenovirus-induced immune responses; consequently, the animals could be re-administered with adenovirus vectors. Short treatment with this Ab resulted in long-term effects on immune functions and prolonged inhibition of the adenovirus-specific humoral response well beyond the time when the Ab effects were no longer significant, pointing to the therapeutic potential in blockade of this costimulatory pathway as an immunomodulatory regimen to enable administration of gene transfer vectors (Scaria et al. 1997 Gene Ther. 4: 611; Chirmule et al 2000 J. Virol. 74: 3345). Other studies showed that co-administration of CTLA4-Ig and an anti-CD40L Ab around the time of primary vector administration decreased immune responses to the vector, prolonged long term adenovirus-mediated gene expression and enabled secondary adenovirus-mediated gene transfer even after the immunosuppressive effects of these agents were no longer present, indicating that it may be possible to obtain persistence as well as secondary adenoviral-mediated gene transfer with transient immunosuppressive therapies (Kay et al 1997 Proc. Natl. Acad. Sci. U.S.A. 94:4686). In another study, similar administration of CTLA4-Ig and an anti-CD40L Ab abrogated the formation of neutralizing Abs against the vector, and enabled gene transfer expression, provided the treatment was administered during each gene transfer injection (Lorain et al 2008 Molecular Therapy 16:541). Furthermore, administration of CTLA4-Ig to mice, even as single administration, resulted in suppression of immune responses and prolonged transgene expression at early time points (Adriouch et al 2011 Front. Microbiol. 2:199). However, CTLA4-Ig alone was not sufficient to permanently wipe out the immune responses against the transgene product. Combined treatment targeting two immune checkpoints with CTLA4-Ig and PD-L1 or PDL-2 resulted in synergistic improvement of transgene tolerance at later time points, by probably targeting two non-redundant mechanisms of immunomodulation, resulting in long term transgene persistence and expression (Adriouch et al 2011 Front. Microbiol. 2:199).

According to at least some embodiments of the present invention, nucleic acid sequences encoding soluble VSTM5 proteins and/or a fusion protein as described herein; alone or in combination with another immunomodulatory agent or in combination with any of the strategies and approaches tested to overcome the limitation of immune responses to gene therapy, could be used for reducing the undesirable immune activation that follows gene therapy.

Current approaches include exclusion of patients with antibodies to the delivery vector, administration of high vector doses, use of empty capsids to adsorb anti-vector antibodies allowing for subsequent vector transduction, repeated plasma exchange (plasmapheresis) cycles to adsorb immunoglobulins and reduce the anti-vector antibody titer.

Novel approaches attempting to overcome these limitations can be divided into two broad categories: selective modification of the Ad vector itself and pre-emptive immune modulation of the host (Seregin and Amalfitano 2010 Viruses 2:2013). The first category comprises several innovative strategies including: (1) Ad-capsid-display of specific inhibitors or ligands; (2) covalent modifications of the entire Ad vector capsid moiety; (3) the use of tissue specific promoters and local administration routes; (4) the use of genome modified Ads; and (5) the development of chimeric or alternative serotype Ads.

The second category of methods includes the use of immunosuppressive drugs or specific compounds to block important immune pathways, which are known to be induced by viral vectors Immunosuppressive agents have been tested in preclinical studies and shown efficacy in prevention or eradication of immune responses to the transfer vector and transgene product. These include general immunosuppressive agents such as cyclosporine A; cyclophosphamide; FK506; glucocorticoids or steroids such as dexamethasone; TLR9 blockade such as the TLR9 antagonist oligonucleotide ODN-2088; TNF-α blockade with anti-TNF-α antibodies or TNFR-Ig antibody, Erk and other signaling inhibitors such as U0126. In the clinical setting, administration of glucocorticoids has been successfully used to blunt T cell responses directed against the viral capsid upon liver gene transfer of adenovirus-associated virus (AAV) vector expressing human factor IX transgene to severe hemophilia B patients (Nathwani et al 2011 N. Engl. J. Med. 365:2357).

In contrast to the previous approaches that utilize drugs that tend to “globally” and non-specifically immunosuppress the host, more selective immunosuppressive approaches have been developed. These include the use of agents which provide blockade of positive co-stimulatory interactions, such as between CD40 and CD154, ICOS and ICOSL, CD28 and CD80 or CD86 (including CTLA4-Ig), NKG2D and NKG2D ligands, LFA-1 and ICAM, LFA-3 and CD2, 4-1BB and 4-1BBL, OX40 and OX40L, GITR and GITRL and agents that stimulate negative costimulatory receptors such as CTLA-4, PD-1, BTLA, LAG-3, TIM-1, TIM-3, KIRs, and the receptors for B7-H4 and B7-H3. Some of these have been utilized in preclinical or clinical transplantation studies (Pilat et al 2011 Sem. Immunol. 23:293).

In the above-described gene or cell therapies or in treating transplant indications preferably a subject who has or is to receive cell or gene therapy or a transplanted tissue or organ will be administered an immunoinhibitory anti-VSTM5 antibody or antigen-binding fragment according to the invention, which antibody enhances, agonizes or mimics at least one VSTM5 mediated effect on immunity, e.g., its inhibitory effect on cytotoxic T cells or NK activity and/or its inhibitory effect on the production of proinflammatory cytokines, or its stimulatory effect on TRegs thereby preventing or reducing host immune responses against the cell or gene used in therapy or an undesired immune response against the transplanted cells, organ or tissue. Preferably the treatment will elicit prolonged immune tolerance against the transplanted or infused cells, tissue or organ. In some instances, e.g., in the case of transplanted cells, tissues or organs containing immune cells, the immunoinhibitory anti-VSTM5 antibody or antigen-binding fragment may be contacted with the cells, tissue or organ prior to infusion or transplant, and/or potentially immune cells of the transplant recipient in order to tolerize the immune cells and potentially prevent an undesired immune response or GVHD immune reaction.

### Pharmaceutical Compositions

In another aspect, the present invention provides a composition, e.g., a pharmaceutical composition, containing one or a combination of the therapeutic agent, according to at least some embodiments of the invention.

Thus, the present invention features a pharmaceutical composition comprising a therapeutically effective amount of a therapeutic agent according to at least some embodiments of the present invention.

The pharmaceutical composition according to at least some embodiments of the present invention is further preferably used for the treatment of cancer, wherein the cancer is non-metastatic, invasive or metastatic, and/or for treatment of immune related disorder, infectious disorder and/or sepsis, as recited herein.

“Treatment” refers to both therapeutic treatment and prophylactic or preventative measures. Those in need of treatment include those already with the disorder as well as those in which the disorder is to be prevented. Hence, the mammal to be treated herein may have been diagnosed as having the disorder or may be predisposed or susceptible to the disorder. “Mammal” for purposes of treatment refers to any animal classified as a mammal, including humans, domestic and farm animals, and zoo, sports, or pet animals, such as dogs, horses, cats, cows, etc. Preferably, the mammal is human.

The term “therapeutically effective amount” refers to an amount of agent according to the present invention that is effective to treat a disease or disorder in a mammal.

The therapeutic agents of the present invention can be provided to the subject alone or as part of a pharmaceutical composition where they are mixed with a pharmaceutically acceptable carrier.

A composition is said to be a “pharmaceutically acceptable carrier” if its administration can be tolerated by a recipient patient. As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion).

Such compositions include sterile water, buffered saline (e.g., Tris-HCl, acetate, phosphate), pH and ionic strength and optionally additives such as detergents and solubilizing agents (e.g., Polysorbate 20, Polysorbate 80), antioxidants (e.g., ascorbic acid, sodium metabisulfite), preservatives (e.g., Thimersol, benzyl alcohol) and bulking substances (e.g., lactose, mannitol). Non-aqueous solvents or vehicles may also be used as detailed below.

Examples of suitable aqueous and nonaqueous carriers that may be employed in the pharmaceutical compositions according to at least some embodiments of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. Depending on the route of administration, the active compound, i.e., monoclonal or polyclonal antibodies and antigen-binding fragments and conjugates containing same, and/or alternative scaffolds, that specifically bind any one of VSTM5 proteins, or bispecific molecule, may be coated in a material to protect the compound from the action of acids and other natural conditions that may inactivate the compound. The pharmaceutical compounds according to at least some embodiments of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydriodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition according to at least some embodiments of the invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, α-tocopherol, and the like; and (3) metal chelating agents, such as citric acid, ethylenediamine tetraacetic acid (EDTA), sorbitol, tartaric acid, phosphoric acid, and the like.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions according to at least some embodiments of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

A composition of the present invention can be administered via one or more routes of administration using one or more of a variety of methods known in the art. As will be appreciated by the skilled artisan, the route and/or mode of administration will vary depending upon the desired results. Preferred routes of administration for therapeutic agents according to at least some embodiments of the invention include intravascular delivery (e.g. injection or infusion), intravenous, intramuscular, intradermal, intraperitoneal, subcutaneous, spinal, oral, enteral, rectal, pulmonary (e.g. inhalation), nasal, topical (including transdermal, buccal and sublingual), intravesical, intravitreal, intraperitoneal, vaginal, brain delivery (e.g. intra-cerebroventricular, intra-cerebral, and convection enhanced diffusion), CNS delivery (e.g. intrathecal, perispinal, and intra-spinal) or parenteral (including subcutaneous, intramuscular, intravenous and intradermal), transmucosal (e.g., sublingual administration), administration or administration via an implant, or other parenteral routes of administration, for example by injection or infusion, or other delivery routes and/or forms of administration known in the art. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. In a specific embodiment, a protein, a therapeutic agent or a pharmaceutical composition according to at least some embodiments of the present invention can be administered intraperitoneally or intravenously.

Alternatively, an VSTM5 specific antibody or can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in a preferred embodiment, a therapeutic composition according to at least some embodiments of the invention can be administered with a needles hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the anti-VSTM5 antibodies can be formulated to ensure proper distribution in vivo. For example, the blood-brain barrier (BBB) excludes many highly hydrophilic compounds. To ensure that the therapeutic compounds according to at least some embodiments of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J Physiol. 1233:134); p120 (Schreier et al. (1994) J. Biol. Chem. 269:9090); see also K. Keinanen; M. L. Laukkanen (1994) FEBS Lett. 346:123; J. J. Killion; and I. J. Fidler (1994) Immunomethods 4:273.

In yet another embodiment, immunoconjugates of the invention can be used to target compounds (e.g., therapeutic agents, labels, cytotoxins, radiotoxins immunosuppressants, etc.) to cells which have VSTM5 cell surface receptors by linking such compounds to the antibody. Thus, the invention also provides methods for localizing ex vivo or in vivo cells expressing VSTM5 (e.g., with a detectable label, such as a radioisotope, a fluorescent compound, an enzyme, or an enzyme co-factor). Alternatively, the immunoconjugates can be used to kill cells which have VSTM5 cell surface receptors by targeting cytotoxins or radiotoxins to VSTM5 antigen.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., soluble polypeptide conjugate containing the ectodomain of the VSTM5 antigen, antibody, immunoconjugate, alternative scaffolds, and/or bispecific molecule, may be coated in a material to protect the compound from the action of acids and other natural conditions that may inactivate the compound. The pharmaceutical compounds according to at least some embodiments of the present invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition according to at least some embodiments of the present invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, α-tocopherol, and the like; and (3) metal chelating agents, such as citric acid, ethylenediamine tetraacetic acid (EDTA), sorbitol, tartaric acid, phosphoric acid, and the like. Examples of suitable aqueous and nonaqueous carriers that may be employed in the pharmaceutical compositions according to at least some embodiments of the present invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions according to at least some embodiments of the present invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about I percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms according to at least some embodiments of the present invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the antibody, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Preferred dosage regimens for an antibody according to at least some embodiments of the present invention include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks.

In some methods, two or more monoclonal antibodies with different binding specificities are administered simultaneously, in which case the dosage of each antibody administered falls within the ranges indicated. Antibody is usually administered on multiple occasions. Intervals between single dosages can be, for example, daily, weekly, monthly, every three months or yearly. Intervals can also be irregular as indicated by measuring blood levels of antibody to the target antigen in the patient. In some methods, dosage is adjusted to achieve a plasma antibody concentration of about 1-1000 mug/ml and in some methods about 25-300 microgram/ml.

Alternatively, therapeutic agent can be administered as a sustained release formulation, in which case less frequent administration is required. Dosage and frequency vary depending on the half-life of the therapeutic agent in the patient. In general, human antibodies show the longest half-life, followed by humanized antibodies, chimeric antibodies, and nonhuman antibodies. The half-life for fusion proteins may vary widely. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

Formulations for Parenteral Administration

In a further embodiment, compositions disclosed herein, including those containing peptides and polypeptides, are administered in an aqueous solution, by parenteral injection. The formulation may also be in the form of a suspension or emulsion. In general, pharmaceutical compositions are provided including effective amounts of a peptide or polypeptide, and optionally include pharmaceutically acceptable diluents, preservatives, solubilizers, emulsifiers, adjuvants and/or carriers. Such compositions optionally include one or more for the following: diluents, sterile water, buffered saline of various buffer content (e.g., Tris-HCl, acetate, phosphate), pH and ionic strength; and additives such as detergents and solubilizing agents (e.g., TWEEN 20® (polysorbate-20), TWEEN 80® (polysorbate-80)), anti-oxidants (e.g., water soluble antioxidants such as ascorbic acid, sodium metabisulfite, cysteine hydrochloride, sodium bisulfate, sodium metabisulfite, sodium sulfite; oil-soluble antioxidants, such as ascorbyl palmitate, butylated hydroxyanisole (BHA), butylated hydroxytoluene (BHT), lecithin, propyl gallate, α-tocopherol; and metal chelating agents, such as citric acid, ethylenediamine tetraacetic acid (EDTA), sorbitol, tartaric acid, phosphoric acid), and preservatives (e.g., Thimersol®, benzyl alcohol) and bulking substances (e.g., lactose, mannitol). Examples of non-aqueous solvents or vehicles are ethanol, propylene glycol, polyethylene glycol, vegetable oils, such as olive oil and corn oil, gelatin, and injectable organic esters such as ethyl oleate. The formulations may be freeze dried (lyophilized) or vacuum dried and redissolved/resuspended immediately before use. The formulation may be sterilized by, for example, filtration through a bacteria retaining filter, by incorporating sterilizing agents into the compositions, by irradiating the compositions, or by heating the compositions.

Formulations for Topical Administration

VSTM5 polypeptides, fragments, fusion polypeptides, nucleic acids, and vectors disclosed herein can be applied topically. Topical administration does not work well for most peptide formulations, although it can be effective especially if applied to the lungs, nasal, oral (sublingual, buccal), vaginal, or rectal mucosa.

Compositions can be delivered to the lungs while inhaling and traverse across the lung epithelial lining to the blood stream when delivered either as an aerosol or spray dried particles having an aerodynamic diameter of less than about 5 microns. A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa will typically be spray dried drug particles, which may be incorporated into a tablet, gel, capsule, suspension or emulsion. Standard pharmaceutical excipients are available from any formulator. Oral formulations may be in the form of chewing gum, gel strips, tablets or lozenges. Transdermal formulations may also be prepared. These will typically be ointments, lotions, sprays, or patches, all of which can be prepared using standard technology. Transdermal formulations will require the inclusion of penetration enhancers.

Controlled Delivery Polymeric Matrices

VSTM5 polypeptides, fragments, fusion polypeptides, nucleic acids, and vectors disclosed herein may also be administered in controlled release formulations. Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of polypeptides or nucleic acids encoding the polypeptides, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl Polymer Sci, 35:755-774 (1988).

The devices can be formulated for local release to treat the area of implantation or injection—which will typically deliver a dosage that is much less than the dosage for treatment of an entire body—or systemic delivery. These can be implanted or injected subcutaneously, into the muscle, fat, or swallowed.

Diagnostic Uses of Anti-VSTM5 Antibodies

According to at least some embodiments of the present invention, the antibodies (e.g., human monoclonal antibodies, multispecific and bispecific molecules and compositions) can be used to detect levels of VSTM5 or levels of cells which contain VSTM5 on their membrane surface, which levels can then be linked to certain disease symptoms. Alternatively, the antibodies can be used to inhibit or block VSTM5 function which, in turn, can be linked to the prevention or amelioration of cancer. This can be achieved by contacting a sample and a control sample with the anti-VSTM5 antibody under conditions that allow for the formation of a complex between the corresponding antibody and VSTM5. Any complexes formed between the antibody and VSTM5 are detected and compared in the sample and the control.

According to at least some embodiments of the present invention, the antibodies (e.g., human antibodies, multispecific and bispecific molecules and compositions) can be initially tested for binding activity associated with therapeutic or diagnostic use in vitro. For example, compositions according to at least some embodiments of the present invention can be tested using low cytometric assays.

Also within the scope of the present invention are kits comprising the VSTM5 specific antibody according to at least some embodiments of the present invention (e.g., human antibodies, alternative scaffolds, bispecific or multispecific molecules, or immunoconjugates) and instructions for use. The kit can further contain one or more additional reagents, such as an immunosuppressive reagent, a cytotoxic agent or a radiotoxic agent, or one or more additional antibodies (including human antibodies) according to at least some embodiments of the present invention (e.g., a human antibody having a complementary activity which binds to an epitope in the antigen distinct from the first human antibody).

The antibodies according to at least some embodiments of the present invention can also be used to target cells expressing FcγR or VSTM5 for example for labeling such cells. For such use, the binding agent can be linked to a molecule that can be detected. Thus, the present invention provides methods for localizing ex vivo or in vitro cells expressing Fc receptors, such as FcγR, or VSTM5 antigen. The detectable label can be, e.g., a radioisotope, a fluorescent compound, an enzyme, or an enzyme co-factor.

In a particular embodiment, the present invention provides methods for detecting the presence and/or level of VSTM5 antigen in a sample, or measuring the amount of VSTM5 antigen, respectively, comprising contacting the sample, and a control sample, with an antibody, or an antigen-binding portion thereof, which specifically binds to VSTM5, under conditions that allow for formation of a complex between the antibody or portion thereof and VSTM5. The formation of a complex is then detected, wherein a difference complex formation between the sample compared to the control sample is indicative the presence of VSTM5 antigen in the sample. As noted the present invention in particular embraces assays for detecting VSTM5 antigen in vitro and in vivo such as immunoassays, radioimmunoassays, radioassays, radioimaging assays, ELISAs, Western blot, FACS, slot blot, immunohistochemical assays, receptor occupancy assays and other assays well known to those skilled in the art.

In yet another embodiment, immunoconjugates of the present invention can be used to target compounds (e.g., therapeutic agents, labels, cytotoxins, radiotoxins immunosuppressants, etc.) to cells which have VSTM5 cell surface receptors by linking such compounds to the antibody. Thus, the present invention also provides methods for localizing ex vivo or in vivo cells expressing VSTM5 (e.g., with a detectable label, such as a radioisotope, a fluorescent compound, an enzyme, or an enzyme co-factor). Alternatively, the immunoconjugates can be used to kill cells which have VSTM5 cell surface receptors by targeting cytotoxins or radiotoxins to VSTM5 antigen.

According to at least some embodiments, the present invention provides a method for imaging an organ or tissue, the method comprising: (a) administering to a subject in need of such imaging, a labeled polypeptide; and (b) detecting the labeled polypeptide to determine where the labeled polypeptide is concentrated in the subject. When used in imaging applications, the labeled polypeptides according to at least some embodiments of the present invention typically have an imaging agent covalently or monovalently attached thereto. Suitable imaging agents include, but are not limited to, radionuclides, detectable tags, fluorophores, fluorescent proteins, enzymatic proteins, and the like. One of skill in the art will be familiar with other methods for attaching imaging agents to polypeptides. For example, the imaging agent can be attached via site-specific conjugation, e.g., covalent attachment of the imaging agent to a peptide linker such as a polyarginine moiety having five to seven arginines present at the carboxyl-terminus of and Fc fusion molecule. The imaging agent can also be directly attached via non-site specific conjugation, e.g., covalent attachment of the imaging agent to primary amine groups present in the polypeptide. One of skill in the art will appreciate that an imaging agent can also be bound to a protein via noncovalent interactions (e.g., ionic bonds, hydrophobic interactions, hydrogen bonds, Van der Waals forces, dipole-dipole bonds, etc.).

In certain instances, the polypeptide is radiolabeled with a radionuclide by directly attaching the radionuclide to the polypeptide. In certain other instances, the radionuclide is bound to a chelating agent or chelating agent-linker attached to the polypeptide. Suitable radionuclides for direct conjugation include, without limitation ¹⁸F, ¹²⁴I, ¹²⁵I, ¹³¹I, and mixtures thereof. Suitable radionuclides for use with a chelating agent include ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and mixtures thereof. Preferably, the radionuclide bound to a chelating agent is ⁶⁴Cu, ⁹⁰Y, ¹¹¹In, or mixtures thereof. Suitable chelating agents include, but are not limited to, DOTA, BAD, TETA, DTPA, EDTA, NTA, HDTA, their phosphonate analogs, and mixtures thereof. One of skill in the art will be familiar with methods for attaching radionuclides, chelating agents, and chelating agent-linkers to polypeptides of the present invention. In particular, attachment can be conveniently accomplished using, for example, commercially available bifunctional linking groups (generally heterobifunctional linking groups) that can be attached to a functional group present in a non-interfering position on the polypeptide and then further linked to a radionuclide, chelating agent, or chelating agent-linker.

Non-limiting examples of fluorophores or fluorescent dyes suitable for use as imaging agents include Alexa Fluor® dyes (Invitrogen Corp.; Carlsbad, Calif.), fluorescein, fluorescein isothiocyanate (FITC), Oregon Green™; rhodamine, Texas red, tetrarhodamine isothiocynate (TRITC), CyDye™ fluors (e.g., Cy2, Cy3, Cy5), and the like.

Examples of fluorescent proteins suitable for use as imaging agents include, but are not limited to, green fluorescent protein, red fluorescent protein (e.g., DsRed), yellow fluorescent protein, cyan fluorescent protein, blue fluorescent protein, and variants thereof (see, e.g., U.S. Pat. Nos. 6,403,374, 6,800,733, and 7,157,566). Specific examples of GFP variants include, but are not limited to, enhanced GFP (EGFP), destabilized EGFP, the GFP variants described in Doan et al., Mol. Microbiol., 55:1767-1781 (2005), the GFP variant described in Crameri et al., Nat. Biotechnol., 14:315-319 (1996), the cerulean fluorescent proteins described in Rizzo et al., Nat. Biotechnol, 22:445 (2004) and Tsien, Annu. Rev. Biochem., 67:509 (1998), and the yellow fluorescent protein described in Nagal et al., Nat. Biotechnol., 20:87-90 (2002). DsRed variants are described in, e.g., Shaner et al., Nat. Biotechnol., 22:1567-1572 (2004), and include mStrawberry, mCherry, mOrange, mBanana, mHoneydew, and mTangerine. Additional DsRed variants are described in, e.g., Wang et al., Proc. Natl. Acad. Sci. U.S.A., 101:16745-16749 (2004) and include mRaspberry and mPlum. Further examples of DsRed variants include mRFPmars described in Fischer et al., FEBS Lett., 577:227-232 (2004) and mRFPruby described in Fischer et al., FEBS Lett., 580:2495-2502 (2006).

In other embodiments, the imaging agent that is bound to a polypeptide according to at least some embodiments of the present invention comprises a detectable tag such as, for example, biotin, avidin, streptavidin, or neutravidin. In further embodiments, the imaging agent comprises an enzymatic protein including, but not limited to, luciferase, chloramphenicol acetyltransferase, β-galactosidase, β-glucuronidase, horseradish peroxidase, xylanase, alkaline phosphatase, and the like.

Any device or method known in the art for detecting the radioactive emissions of radionuclides in a subject is suitable for use in the present invention. For example, methods such as Single Photon Emission Computerized Tomography (SPECT), which detects the radiation from a single photon γ-emitting radionuclide using a rotating γ camera, and radionuclide scintigraphy, which obtains an image or series of sequential images of the distribution of a radionuclide in tissues, organs, or body systems using a scintillation γ camera, may be used for detecting the radiation emitted from a radiolabeled polypeptide of the present invention. Positron emission tomography (PET) is another suitable technique for detecting radiation in a subject. Miniature and flexible radiation detectors intended for medical use are produced by Intra-Medical LLC (Santa Monica, Calif.). Magnetic Resonance Imaging (MRI) or any other imaging technique known to one of skill in the art is also suitable for detecting the radioactive emissions of radionuclides. Regardless of the method or device used, such detection is aimed at determining where the labeled polypeptide is concentrated in a subject, with such concentration being an indicator of disease activity.

Non-invasive fluorescence imaging of animals and humans can also provide in vivo diagnostic information and be used in a wide variety of clinical specialties. For instance, techniques have been developed over the years for simple ocular observations following UV excitation to sophisticated spectroscopic imaging using advanced equipment (see, e.g., Andersson-Engels et al., Phys. Med. Biol., 42:815-824 (1997)). Specific devices or methods known in the art for the in vivo detection of fluorescence, e.g., from fluorophores or fluorescent proteins, include, but are not limited to, in vivo near-infrared fluorescence (see, e.g., Frangioni, Curr. Opin. Chem. Biol., 7:626-634 (2003)), the Maestro™ in vivo fluorescence imaging system (Cambridge Research & Instrumentation, Inc.; Woburn, Mass.), in vivo fluorescence imaging using a flying-spot scanner (see, e.g., Ramanujam et al., IEEE Transactions on Biomedical Engineering, 48:1034-1041 (2001), and the like.

Other methods or devices for detecting an optical response include, without limitation, visual inspection, CCD cameras, video cameras, photographic film, laser-scanning devices, fluorometers, photodiodes, quantum counters, epifluorescence microscopes, scanning microscopes, flow cytometers, fluorescence microplate readers, or signal amplification using photomultiplier tubes.

According to some embodiments, the sample taken from a subject (patient) to perform the diagnostic assay according to at least some embodiments of the present invention is selected from the group consisting of a body fluid or secretion including but not limited to blood, serum, urine, plasma, prostatic fluid, seminal fluid, semen, the external secretions of the skin, respiratory, intestinal, and genitourinary tracts, tears, cerebrospinal fluid, synovial fluid, sputum, saliva, milk, peritoneal fluid, pleural fluid, cyst fluid, secretions of the breast ductal system (and/or lavage thereof), broncho alveolar lavage, lavage of the reproductive system, bone marrow aspiration and lavage of any other part of the body or system in the body; samples of any organ including isolated cells or tissues, wherein the cell or tissue can be obtained from an organ selected from, but not limited to lung, colon, ovarian, lymphatic system, bone marrow, hematopoietic system and/or breast tissue; stool or a tissue sample, or any combination thereof. In some embodiments, the term encompasses samples of in vivo cell culture constituents. Prior to be subjected to the diagnostic assay, the sample can optionally be diluted with a suitable eluant.

In some embodiments, the phrase “marker” in the context of the present invention refers to a nucleic acid fragment, a peptide, or a polypeptide, which is differentially present in a sample taken from patients (subjects) having one of the herein-described diseases or conditions, as compared to a comparable sample taken from subjects who do not have one the above-described diseases or conditions.

In some embodiments, the phrase “differentially present” refers to differences in the quantity or quality of a marker present in a sample taken from patients having one of the herein-described diseases or conditions as compared to a comparable sample taken from patients who do not have one of the herein-described diseases or conditions. For example, a nucleic acid fragment may optionally be differentially present between the two samples if the amount of the nucleic acid fragment in one sample is significantly different from the amount of the nucleic acid fragment in the other sample, for example as measured by hybridization and/or NAT-based assays. A polypeptide is differentially present between the two samples if the amount of the polypeptide in one sample is significantly different from the amount of the polypeptide in the other sample. It should be noted that if the marker is detectable in one sample and not detectable in the other, then such a marker can be considered to be differentially present. Optionally, a relatively low amount of up-regulation may serve as the marker, as described herein. One of ordinary skill in the art could easily determine such relative levels of the markers; further guidance is provided in the description of each individual marker below.

In some embodiments, the phrase “diagnostic” means identifying the presence or nature of a pathologic condition and/or monitoring disease progression and/or monitoring disease response. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein the term “diagnosis” refers to the process of identifying a medical condition or disease by its signs, symptoms, and in particular from the results of various diagnostic procedures, including e.g. detecting the expression of the nucleic acids or polypeptides according to at least some embodiments of the invention in a biological sample (e.g. in cells, tissue or serum, as defined below) obtained from an individual. Furthermore, as used herein the term “diagnosis” encompasses screening for a disease, detecting a presence or a severity of a disease, providing prognosis of a disease, monitoring disease progression or relapse, as well as assessment of treatment efficacy and/or relapse of a disease, disorder or condition, as well as selecting a therapy and/or a treatment for a disease, optimization of a given therapy for a disease, monitoring the treatment of a disease, and/or predicting the suitability of a therapy for specific patients or subpopulations or determining the appropriate dosing of a therapeutic product in patients or subpopulations. The diagnostic procedure can be performed in vivo or in vitro.

In some embodiments, the phrase “qualitative” when in reference to differences in expression levels of a polynucleotide or polypeptide as described herein, refers to the presence versus absence of expression, or in some embodiments, the temporal regulation of expression, or in some embodiments, the timing of expression, or in some embodiments, any post-translational modifications to the expressed molecule, and others, as will be appreciated by one skilled in the art. In some embodiments, the phrase “quantitative” when in reference to differences in expression levels of a polynucleotide or polypeptide as described herein, refers to absolute differences in quantity of expression, as determined by any means, known in the art, or in other embodiments, relative differences, which may be statistically significant, or in some embodiments, when viewed as a whole or over a prolonged period of time, etc., indicate a trend in terms of differences in expression.

In some embodiments, the term “diagnosing” refers to classifying a disease or a symptom, determining a severity of the disease, monitoring disease progression, forecasting an outcome of a disease and/or prospects of recovery. The term “detecting” may also optionally encompass any of the above.

Diagnosis of a disease according to the present invention can, in some embodiments, be affected by determining a level of a polynucleotide or a polypeptide of the present invention in a biological sample obtained from the subject, wherein the level determined can be correlated with predisposition to, or presence or absence of the disease.

It should be noted that a “biological sample obtained from the subject” may also optionally comprise a sample that has not been physically removed from the subject, as described in greater detail below.

In some embodiments, the term “level” refers to expression levels of RNA and/or protein or to DNA copy number of a marker of the present invention.

Typically the level of the marker in a biological sample obtained from the subject is different (i.e., increased or decreased) from the level of the same marker in a similar sample obtained from a healthy individual (examples of biological samples are described herein).

Numerous well known tissue or fluid collection methods can be utilized to collect the biological sample from the subject in order to determine the level of DNA, RNA and/or polypeptide of the marker of interest in the subject.

Examples include, but are not limited to, fine needle biopsy, needle biopsy, core needle biopsy and surgical biopsy (e.g., brain biopsy), and lavage. Regardless of the procedure employed, once a biopsy/sample is obtained the level of the marker can be determined and a diagnosis can thus be made.

Determining the level of the same marker in normal tissues of the same origin is preferably effected along-side to detect an elevated expression and/or amplification and/or a decreased expression, of the marker as opposed to the normal tissues.

In some embodiments, the term “test amount” of a marker refers to an amount of a marker in a subject's sample that is consistent with a diagnosis of a particular disease or condition. A test amount can be either in absolute amount (e.g., microgram/ml) or a relative amount (e.g., relative intensity of signals).

In some embodiments, the term “control amount” of a marker can be any amount or a range of amounts to be compared against a test amount of a marker. For example, a control amount of a marker can be the amount of a marker in a patient with a particular disease or condition or a person without such a disease or condition. A control amount can be either in absolute amount (e.g., microgram/mi) or a relative amount (e.g., relative intensity of signals).

In some embodiments, the term “detect” refers to identifying the presence, absence or amount of the object to be detected.

In some embodiments, the term “label” includes any moiety or item detectable by spectroscopic, photo chemical, biochemical, immunochemical, or chemical means. For example, useful labels include ³²P, ³⁵S, fluorescent dyes, electron-dense reagents, enzymes (e.g., as commonly used in an ELISA), biotin-streptavidin, digoxigenin, haptens and proteins for which antisera or monoclonal antibodies are available, or nucleic acid molecules with a sequence complementary to a target. The label often generates a measurable signal, such as a radioactive, chromogenic, or fluorescent signal, that can be used to quantify the amount of bound label in a sample. The label can be incorporated in or attached to a primer or probe either covalently, or through ionic, van der Waals or hydrogen bonds, e.g., incorporation of radioactive nucleotides, or biotinylated nucleotides that are recognized by streptavidin. The label may be directly or indirectly detectable. Indirect detection can involve the binding of a second label to the first label, directly or indirectly. For example, the label can be the ligand of a binding partner, such as biotin, which is a binding partner for streptavidin, or a nucleotide sequence, which is the binding partner for a complementary sequence, to which it can specifically hybridize. The binding partner may itself be directly detectable, for example, an antibody may be itself labeled with a fluorescent molecule. The binding partner also may be indirectly detectable, for example, a nucleic acid having a complementary nucleotide sequence can be a part of a branched DNA molecule that is in turn detectable through hybridization with other labeled nucleic acid molecules (see, e.g., P. D. Fahrlander and A. Klausner, Bio/Technology 6:1165 (1988)). Quantitation of the signal is achieved by, e.g., scintillation counting, densitometry, or flow cytometry.

Exemplary detectable labels, optionally and preferably for use with immunoassays, include but are not limited to magnetic beads, fluorescent dyes, radiolabels, enzymes (e.g., horse radish peroxide, alkaline phosphatase and others commonly used in an ELISA), and calorimetric labels such as colloidal gold or colored glass or plastic beads. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

“Immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” or “specifically interacts or binds” when referring to a protein or peptide (or other epitope), refers, in some embodiments, to a binding reaction that is determinative of the presence of the protein in a heterogeneous population of proteins and other biologics. Thus, under designated immunoassay conditions, the specified antibodies bind to a particular protein at least two times greater than the background (non-specific signal) and do not substantially bind in a significant amount to other proteins present in the sample. Specific binding to an antibody under such conditions may require an antibody that is selected for its specificity for a particular protein. For example, polyclonal antibodies raised to seminal basic protein from specific species such as rat, mouse, or human can be selected to obtain only those polyclonal antibodies that are specifically immunoreactive with seminal basic protein and not with other proteins, except for polymorphic variants and alleles of seminal basic protein. This selection may be achieved by subtracting out antibodies that cross-react with seminal basic protein molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

In another embodiment, this invention provides a method for detecting the polypeptides of this invention in a biological sample, comprising: contacting a biological sample with an antibody specifically recognizing a polypeptide according to the present invention and detecting said interaction; wherein the presence of an interaction correlates with the presence of a polypeptide in the biological sample.

In some embodiments of the present invention, the polypeptides described herein are non-limiting examples of markers for diagnosing a disease and/or an indicative condition. Each marker of the present invention can be used alone or in combination, for various uses, including but not limited to, prognosis, prediction, screening, early diagnosis, determination of progression, therapy selection and treatment monitoring of a disease and/or an indicative condition.

Each polypeptide/polynucleotide of the present invention can be used alone or in combination, for various uses, including but not limited to, prognosis, prediction, screening, early diagnosis, determination of progression, therapy selection and treatment monitoring of disease and/or an indicative condition, as detailed above.

Such a combination may optionally comprise any subcombination of markers, and/or a combination featuring at least one other marker, for example a known marker. Furthermore, such a combination may optionally and preferably be used as described above with regard to determining a ratio between a quantitative or semi-quantitative measurement of any marker described herein to any other marker described herein, and/or any other known marker, and/or any other marker.

In some embodiments of the present invention, there are provided of methods, uses, devices and assays for the diagnosis of a disease or condition. Optionally a plurality of markers may be used with the present invention. The plurality of markers may optionally include a markers described herein, and/or one or more known markers. The plurality of markers is preferably then correlated with the disease or condition. For example, such correlating may optionally comprise determining the concentration of each of the plurality of markers, and individually comparing each marker concentration to a threshold level. Optionally, if the marker concentration is above or below the threshold level (depending upon the marker and/or the diagnostic test being performed), the marker concentration correlates with the disease or condition. Optionally and preferably, a plurality of marker concentrations correlates with the disease or condition.

Alternatively, such correlating may optionally comprise determining the concentration of each of the plurality of markers, calculating a single index value based on the concentration of each of the plurality of markers, and comparing the index value to a threshold level.

Also alternatively, such correlating may optionally comprise determining a temporal change in at least one of the markers, and wherein the temporal change is used in the correlating step.

Also alternatively, such correlating may optionally comprise determining whether at least “X” number of the plurality of markers has a concentration outside of a predetermined range and/or above or below a threshold (as described above). The value of “X” may optionally be one marker, a plurality of markers or all of the markers; alternatively or additionally, rather than including any marker in the count for “X”, one or more specific markers of the plurality of markers may optionally be required to correlate with the disease or condition (according to a range and/or threshold).

Also alternatively, such correlating may optionally comprise determining whether a ratio of marker concentrations for two markers is outside a range and/or above or below a threshold. Optionally, if the ratio is above or below the threshold level and/or outside a range, the ratio correlates with the disease or condition.

Optionally, a combination of two or more these correlations may be used with a single panel and/or for correlating between a plurality of panels.

Optionally, the method distinguishes a disease or condition with a sensitivity of at least 70% at a specificity of at least 85% when compared to normal subjects. As used herein, sensitivity relates to the number of positive (diseased) samples detected out of the total number of positive samples present; specificity relates to the number of true negative (non-diseased) samples detected out of the total number of negative samples present. Preferably, the method distinguishes a disease or condition with a sensitivity of at least 80% at a specificity of at least 90% when compared to normal subjects. More preferably, the method distinguishes a disease or condition with a sensitivity of at least 90% at a specificity of at least 90% when compared to normal subjects. Also more preferably, the method distinguishes a disease or condition with a sensitivity of at least 70% at a specificity of at least 85% when compared to subjects exhibiting symptoms that mimic disease or condition symptoms.

A marker panel may be analyzed in a number of fashions well known to those of skill in the art. For example, each member of a panel may be compared to a “normal” value, or a value indicating a particular outcome. A particular diagnosis/prognosis may depend upon the comparison of each marker to this value; alternatively, if only a subset of markers is outside of a normal range, this subset may be indicative of a particular diagnosis/prognosis. The skilled artisan will also understand that diagnostic markers, differential diagnostic markers, prognostic markers, time of onset markers, disease or condition differentiating markers, etc., may be combined in a single assay or device. Markers may also be commonly used for multiple purposes by, for example, applying a different threshold or a different weighting factor to the marker for the different purposes.

In one embodiment, the panels comprise markers for the following purposes: diagnosis of a disease; diagnosis of disease and indication if the disease is in an acute phase and/or if an acute attack of the disease has occurred; diagnosis of disease and indication if the disease is in a non-acute phase and/or if a non-acute attack of the disease has occurred; indication whether a combination of acute and non-acute phases or attacks has occurred; diagnosis of a disease and prognosis of a subsequent adverse outcome; diagnosis of a disease and prognosis of a subsequent acute or non-acute phase or attack; disease progression (for example for cancer, such progression may include for example occurrence or recurrence of metastasis).

The above diagnoses may also optionally include differential diagnosis of the disease to distinguish it from other diseases, including those diseases that may feature one or more similar or identical symptoms.

In certain embodiments, one or more diagnostic or prognostic indicators are correlated to a condition or disease by merely the presence or absence of the indicators. In other embodiments, threshold levels of a diagnostic or prognostic indicator can be established, and the level of the indicators in a patient sample can simply be compared to the threshold levels. The sensitivity and specificity of a diagnostic and/or prognostic test depends on more than just the analytical “quality” of the test—they also depend on the definition of what constitutes an abnormal result. In practice, Receiver Operating Characteristic curves, or “ROC” curves, are typically calculated by plotting the value of a variable versus its relative frequency in “normal” and “disease” populations, and/or by comparison of results from a subject before, during and/or after treatment.

The present invention also relates to kits based upon such diagnostic methods or assays. Also within the scope of the present invention are kits comprising VSTM5 conjugates or antibody compositions of the invention (e.g., human antibodies, bispecific or multispecific molecules, or immunoconjugates) and instructions for use. The kit can further contain one or more additional reagents, such as an immunosuppressive reagent, a cytotoxic agent or a radiotoxic agent, or one or more additional human antibodies according to at least some embodiments of the invention (e.g., a human antibody having a complementary activity which binds to an epitope in the antigen distinct from the first human antibody).

Immunoassays

In another embodiment of the present invention, an immunoassay can be used to qualitatively or quantitatively detect and analyze markers in a sample. This method comprises: providing an antibody that specifically binds to a marker; contacting a sample with the antibody; and detecting the presence of a complex of the antibody bound to the marker in the sample.

To prepare an antibody that specifically binds to a marker, purified protein markers can be used. Antibodies that specifically bind to a protein marker can be prepared using any suitable methods known in the art.

After the antibody is provided, a marker can be detected and/or quantified using any of a number of well recognized immunological binding assays. Useful assays include, for example, an enzyme immune assay (EIA) such as enzyme-linked immunosorbent assay (ELISA), a radioimmune assay (RIA), a Western blot assay, or a slot blot assay see, e.g., U.S. Pat. Nos. 4,366,241; 4,376,110; 4,517,288; and 4,837,168). Generally, a sample obtained from a subject can be contacted with the antibody that specifically binds the marker.

Optionally, the antibody can be fixed to a solid support to facilitate washing and subsequent isolation of the complex, prior to contacting the antibody with a sample. Examples of solid supports include but are not limited to glass or plastic in the form of, e.g., a microtiter plate, a stick, a bead, or a microbead. Antibodies can also be attached to a solid support.

After incubating the sample with antibodies, the mixture is washed and the antibody-marker complex formed can be detected. This can be accomplished by incubating the washed mixture with a detection reagent. Alternatively, the marker in the sample can be detected using an indirect assay, wherein, for example, a second, labeled antibody is used to detect bound marker-specific antibody, and/or in a competition or inhibition assay wherein, for example, a monoclonal antibody which binds to a distinct epitope of the marker are incubated simultaneously with the mixture.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, marker, volume of solution, concentrations and the like. Usually the assays will be carried out at ambient temperature, although they can be conducted over a range of temperatures, such as 10° C. to 40° C.

The immunoassay can be used to determine a test amount of a marker in a sample from a subject. First, a test amount of a marker in a sample can be detected using the immunoassay methods described above. If a marker is present in the sample, it will form an antibody-marker complex with an antibody that specifically binds the marker under suitable incubation conditions described above. The amount of an antibody-marker complex can optionally be determined by comparing to a standard. As noted above, the test amount of marker need not be measured in absolute units, as long as the unit of measurement can be compared to a control amount and/or signal.

Radio-Immunoassay (RIA):

In one version, this method involves precipitation of the desired substrate and in the methods detailed herein below, with a specific antibody and radiolabeled antibody binding protein (e.g., protein A labeled with 1125) immobilized on a precipitable carrier such as agarose beads. The number of counts in the precipitated pellet is proportional to the amount of substrate.

In an alternate version of the RIA, a labeled substrate and an unlabeled antibody binding protein are employed. A sample containing an unknown amount of substrate is added in varying amounts. The decrease in precipitated counts from the labeled substrate is proportional to the amount of substrate in the added sample.

Enzyme Linked Immunosorbent Assay (ELISA):

This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western Blot:

This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Immunohistochemical Analysis:

This method involves detection of a substrate in situ in fixed cells by substrate specific antibodies. The substrate specific antibodies may be enzyme linked or linked to fluorophores. Detection is by microscopy and subjective evaluation. If enzyme linked antibodies are employed, a colorimetric reaction may be required.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Radio-Imaging Methods

These methods include but are not limited to, positron emission tomography (PET) single photon emission computed tomography (SPECT). Both of these techniques are non-invasive, and can be used to detect and/or measure a wide variety of tissue events and/or functions, such as detecting cancerous cells for example. Unlike PET, SPECT can optionally be used with two labels simultaneously. SPECT has some other advantages as well, for example with regard to cost and the types of labels that can be used. For example, U.S. Pat. No. 6,696,686 describes the use of SPECT for detection of breast cancer, and is hereby incorporated by reference as if fully set forth herein.

Theranostics:

The term theranostics describes the use of diagnostic testing to diagnose the disease, choose the correct treatment regime according to the results of diagnostic testing and/or monitor the patient response to therapy according to the results of diagnostic testing. Theranostic tests can be used to select patients for treatments that are particularly likely to benefit them and unlikely to produce side-effects. They can also provide an early and objective indication of treatment efficacy in individual patients, so that (if necessary) the treatment can be altered with a minimum of delay. For example: DAKO and Genentech together created HercepTest® and Herceptin® (trastuzumab) for the treatment of breast cancer, the first theranostic test approved simultaneously with a new therapeutic drug. In addition to HercepTest® (which is an immunohistochemical test), other theranostic tests are in development which use traditional clinical chemistry, immunoassay, cell-based technologies and nucleic acid tests. PPGx's recently launched TPMT (thiopurine S-methyltransferase) test, which is enabling doctors to identify patients at risk for potentially fatal adverse reactions to 6-mercaptopurine, an agent used in the treatment of leukemia. Also, Nova Molecular pioneered SNP genotyping of the apolipoprotein E gene to predict Alzheimer's disease patients' responses to cholinomimetic therapies and it is now widely used in clinical trials of new drugs for this indication. Thus, the field of theranostics represents the intersection of diagnostic testing information that predicts the response of a patient to a treatment with the selection of the appropriate treatment for that particular patient.

As described herein, the term “theranostic” may optionally refer to first testing the subject, such as the patient, for a certain minimum level of VSTM5, for example optionally in the cancerous tissue and/or in the immune infiltrate, as described herein as a sufficient level of VSTM5 expression. Testing may optionally be performed ex vivo, in which the sample is removed from the subject, or in vivo.

If the cancerous tissue and/or the immune infiltrate have been shown to have the minimum level of VSTM5, then an anti-VSTM5 antibody, alone or optionally with other treatment modalities as described herein, may optionally be administered to the subject.

Surrogate Markers:

A surrogate marker is a marker, that is detectable in a laboratory and/or according to a physical sign or symptom on the patient, and that is used in therapeutic trials as a substitute for a clinically meaningful endpoint. The surrogate marker is a direct measure of how a patient feels, functions, or survives which is expected to predict the effect of the therapy. The need for surrogate markers mainly arises when such markers can be measured earlier, more conveniently, or more frequently than the endpoints of interest in terms of the effect of a treatment on a patient, which are referred to as the clinical endpoints. Ideally, a surrogate marker should be biologically plausible, predictive of disease progression and measurable by standardized assays (including but not limited to traditional clinical chemistry, immunoassay, cell-based technologies, receptor occupancy assay nucleic acid tests and imaging modalities).

The therapeutic compositions (e.g., human antibodies, multispecific and bispecific molecules and immunoconjugates) according to at least some embodiments of the invention which have complement binding sites, such as portions from IgG1, IgG2, or IgG3 or IgM which bind complement, can also be used in the presence of complement. In one embodiment, ex vivo treatment of a population of cells comprising target cells with a binding agent according to at least some embodiments of the invention and appropriate effector cells can be supplemented by the addition of complement or serum containing complement. Phagocytosis of target cells coated with a binding agent according to at least some embodiments of the invention can be improved by binding of complement proteins. In another embodiment target cells coated with the compositions (e.g., human antibodies, multispecific and bispecific molecules) according to at least some embodiments of the invention can also be lysed by complement. In yet another embodiment, the compositions according to at least some embodiments of the invention do not activate complement. The therapeutic compositions (e.g., human antibodies, multispecific and bispecific molecules and immunoconjugates) according to at least some embodiments of the invention can also be administered together with complement. Thus, according to at least some embodiments of the invention there are compositions, comprising human antibodies, multispecific or bispecific molecules and serum or complement. These compositions are advantageous in that the complement is located in close proximity to the human antibodies, multispecific or bispecific molecules. Alternatively, the human antibodies, multispecific or bispecific molecules according to at least some embodiments of the invention and the complement or serum can be administered separately.

In one aspect, the invention provides a method for determining whether an anti-VSTM5 antibody has produced a desired immunomodulatory effect in a human (e.g., a cancer patient). The method includes detecting an increase or decrease of at least one immunomodulatory biomarker (sometimes referred to herein as an “anti-VSTM5 antibody-associated immunomodulatory biomarker”) described herein in a blood sample obtained from a patient who has been administered an anti-VSTM5 antibody to thereby determine whether the anti-VSTM5 antibody has produced an immunomodulatory effect. The immunomodulatory effect can be characterized by a change (e.g., an increase or a decrease) in at least one biomarker, e.g., an anti-VSTM5 antibody-associated immunomodulatory biomarker described herein, the change selected from the group consisting of: (i) a reduced concentration of regulatory T cells, relative to the concentration of regulatory T cells of the same histological type in the human prior to the first administration of the antibody; (ii) an increased concentration of CTL cells, relative to the concentration of CTL cells of the same histological type in the human prior to the first administration of the antibody; (iii) an increased concentration of activated T cells, relative to the concentration of activated T cells of the same histological type in the human prior to the first administration of the antibody; (iv) an increased concentration of NK cells, relative to the concentration of NK cells of the same histological type in the human prior to the first administration of the antibody; (v) a ratio of percent activated T cells to percent regulatory T cells (T regs) of at least 2:1 (e.g., at least 3:1, at least 4:1, at least 5:1, at least 6:1, or at least 7:1), relative to the ratio of activated T cells to T regs in the human prior to the first administration of the antibody; (vii) a changed level of VSTM5 expression by a plurality of leukocytes in a biological sample obtained from a patient prior to administration to the patient of an anti-VSTM5 antibody, relative to the level of VSTM5 expression by a plurality of leukocytes of the same histological type in a biological sample from the patient prior to administration of the antibody;

It is understood that in some embodiments, a change in expression can be a change in protein expression or a change in mRNA expression. That is, for example, the methods can interrogate a population of leukocytes from a patient to determine if a reduction in the level of VSTM5 mRNA and/or VSTM5 protein expression has occurred, relative to a control level of mRNA and/or protein expression. Methods for measuring protein and mRNA expression are well known in the art and described herein.

In some embodiments, any of the methods described herein (e.g., the methods for determining whether an anti-VSTM5 has produced a desired immunomodulatory effect in a human) can include measuring the concentration of the specified cell type (e.g. CD4⁺ T cells, CTLs, NK cells etc.), or quantifying the level of expression of a specified expression marker on a specified cell type (e.g. Foxp3, CD25, CD69, etc.), in a biological sample obtained from the human prior to administration of the antibody.

### EXAMPLES

The present invention is further illustrated by the following examples. This information and examples is illustrative and should not be construed as further limiting. The contents of all figures and all references, patents and published patent applications cited throughout this application are expressly incorporated herein by reference.

### EXAMPLES

### Example 1

### IHC Analysis of VSTM5 Proteins

In order to evaluate VSTM5 expression in cancer and normal tissues several IHC studies were performed using FFPE (Formalin-Fixed, Paraffin-Embedded) samples or TMAs (Tissue MicroArray) by Asterand (Royston, UK).

Tissue Details: ‘Multi-Tumor’ TMA

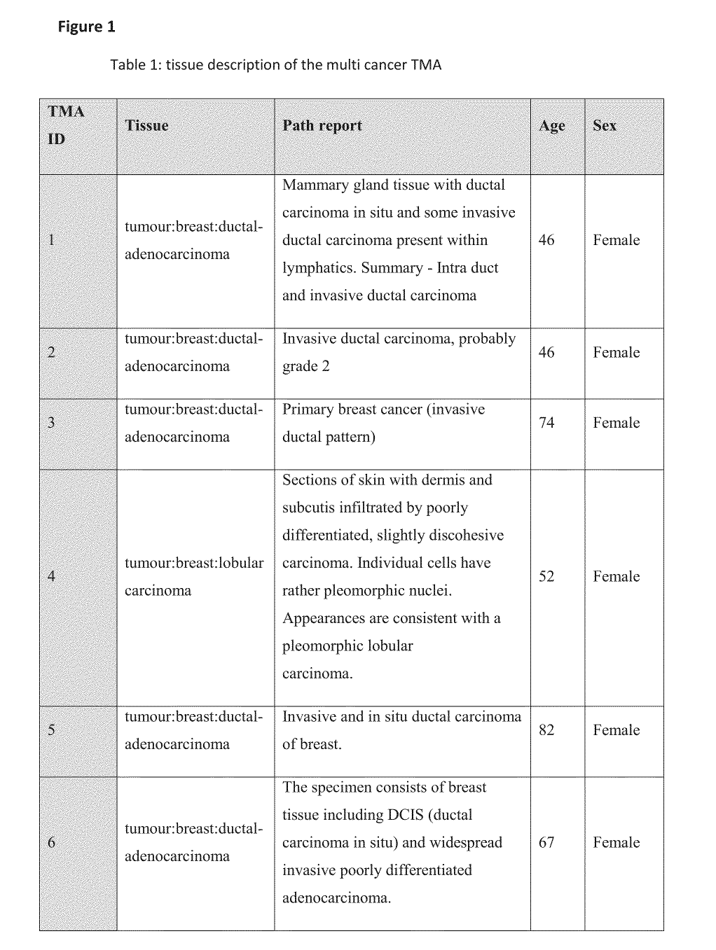

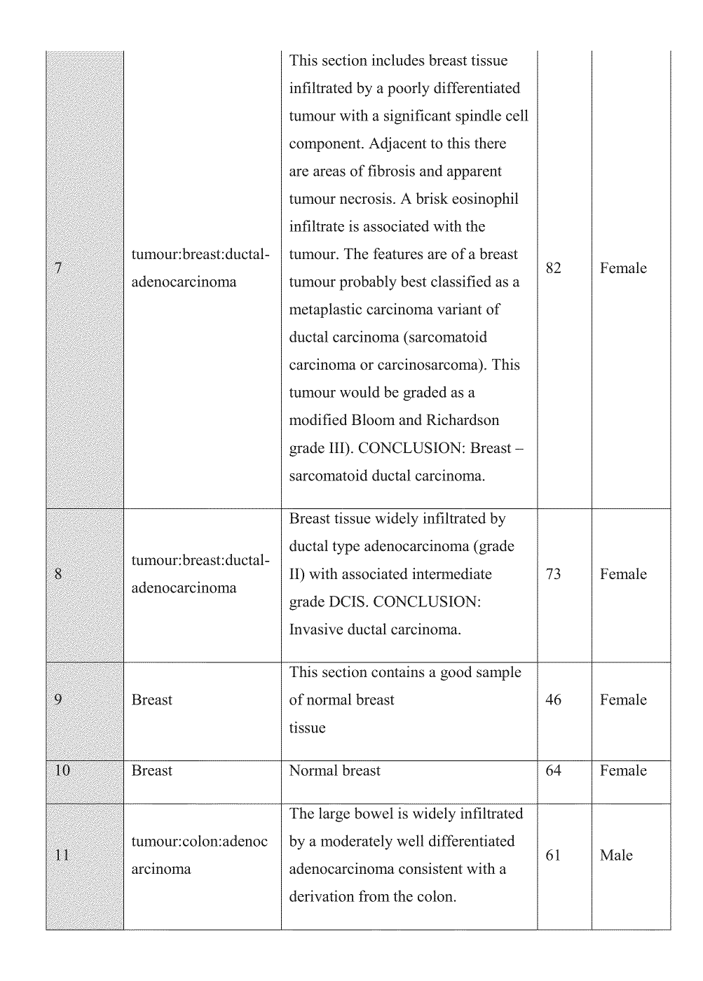

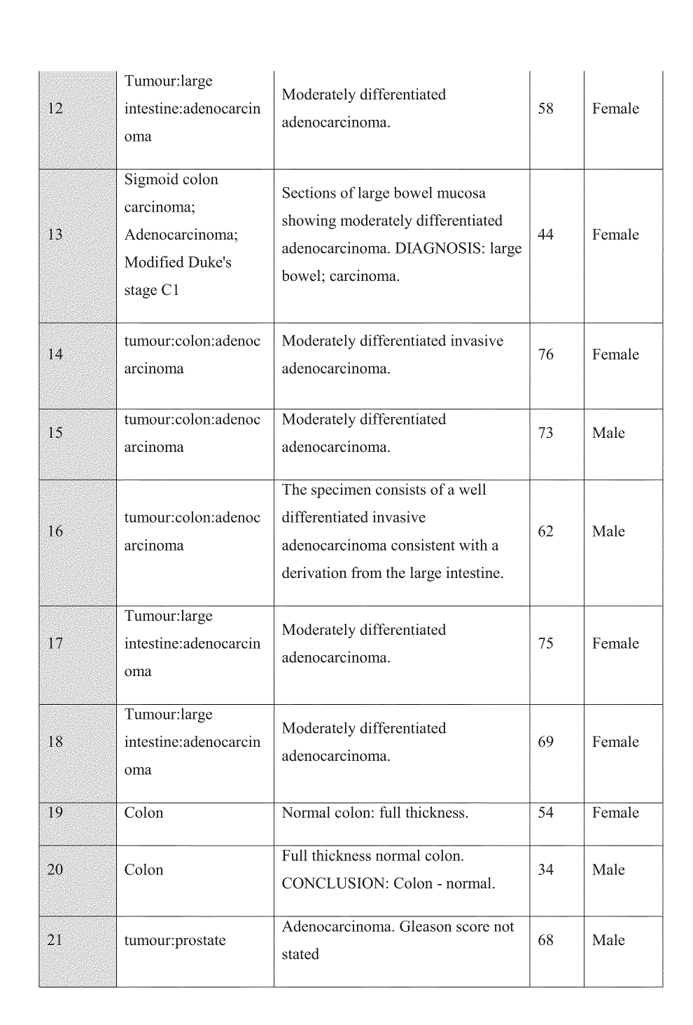

As described in detail in FIG. 1 the TMA comprised 11 tissue types: breast, colon, lymphoid and prostate (8 tumor and 2 normal samples of each), gastric, ovary, brain, kidney, liver and skin (4 tumor and 2 normal samples of each), and lung (8 non-small cell tumor and 4 small cell tumor samples, and 4 normal lung samples).

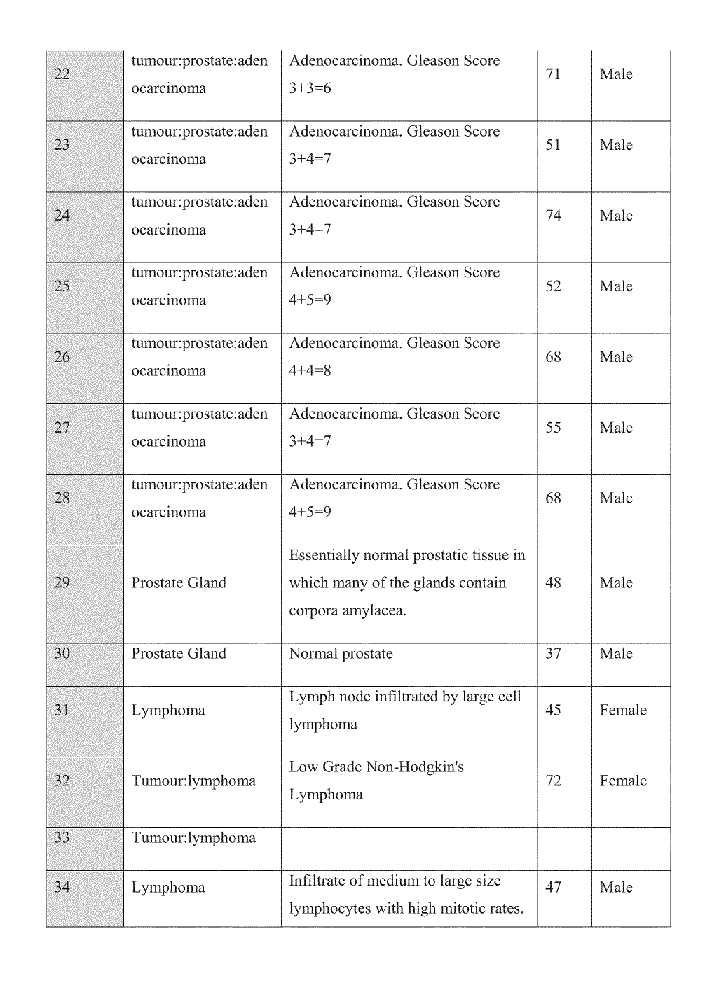

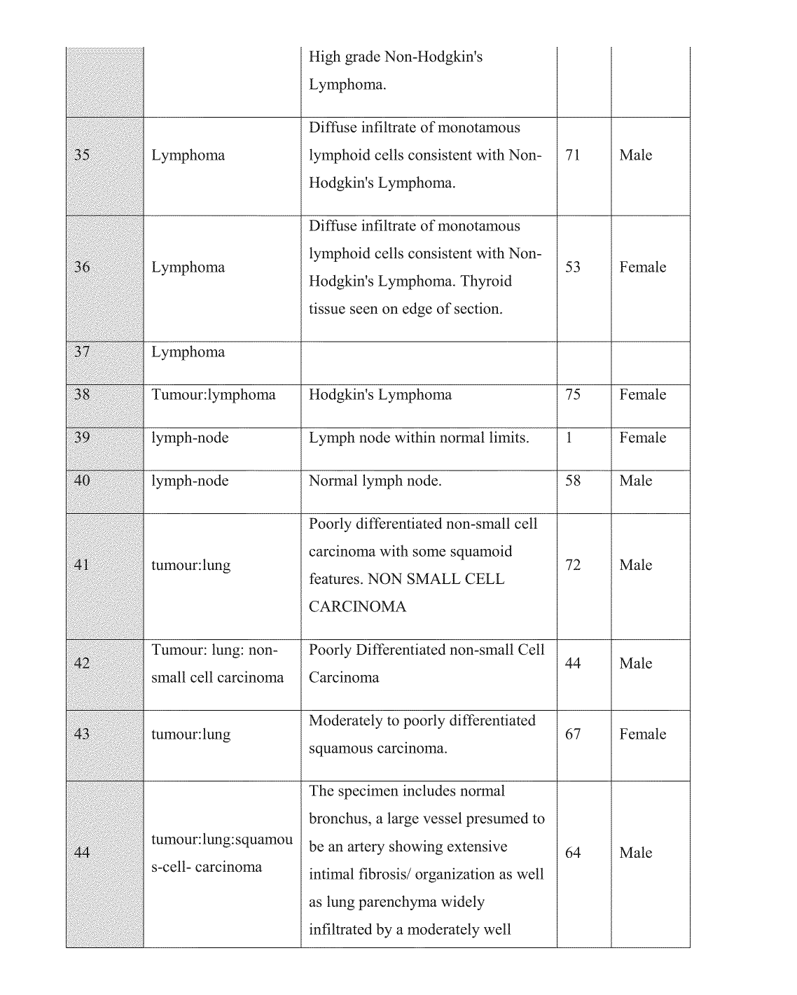

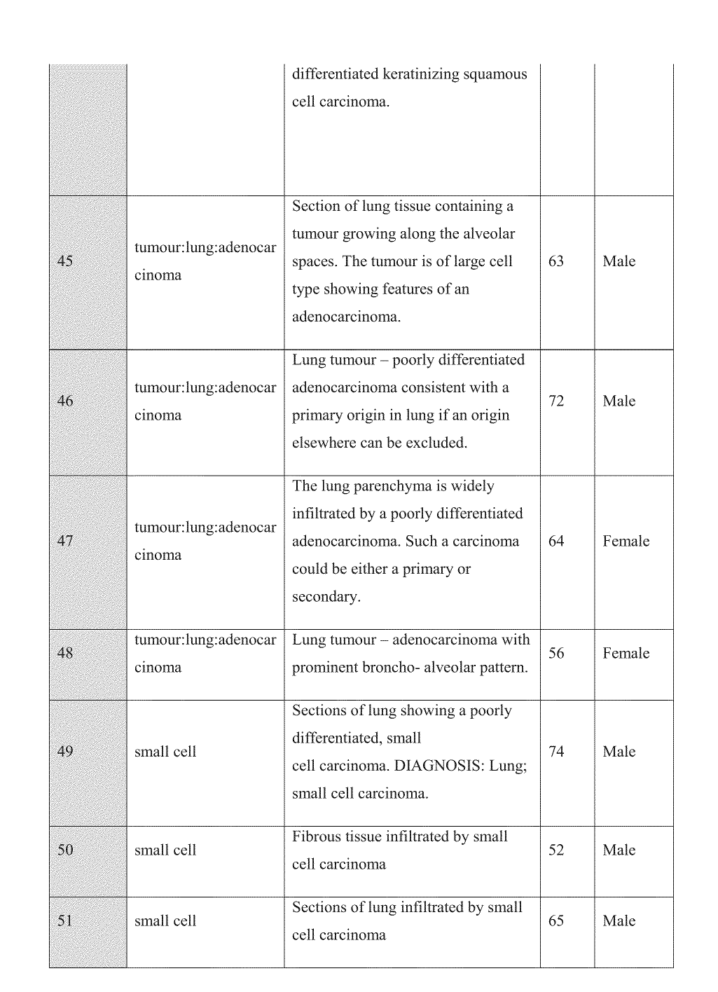

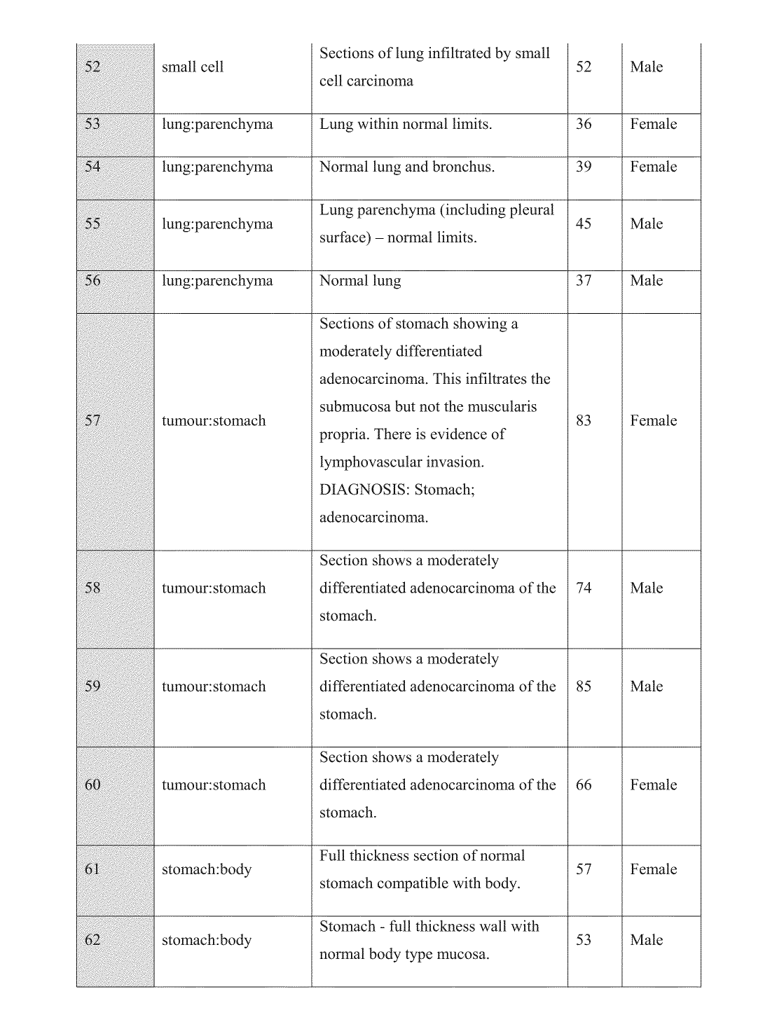

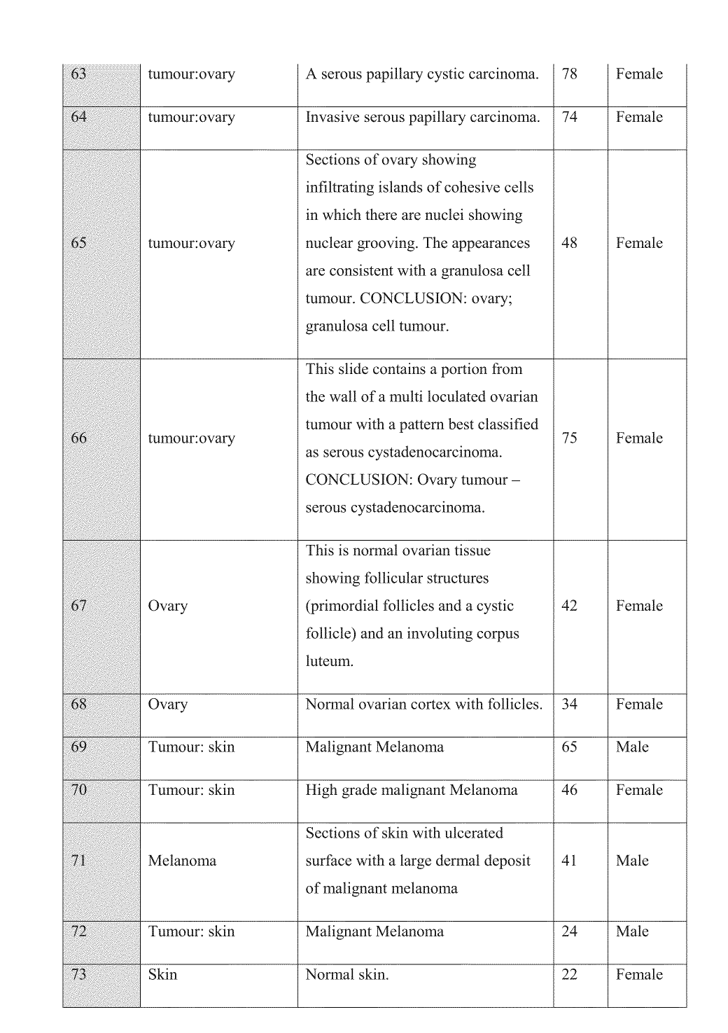

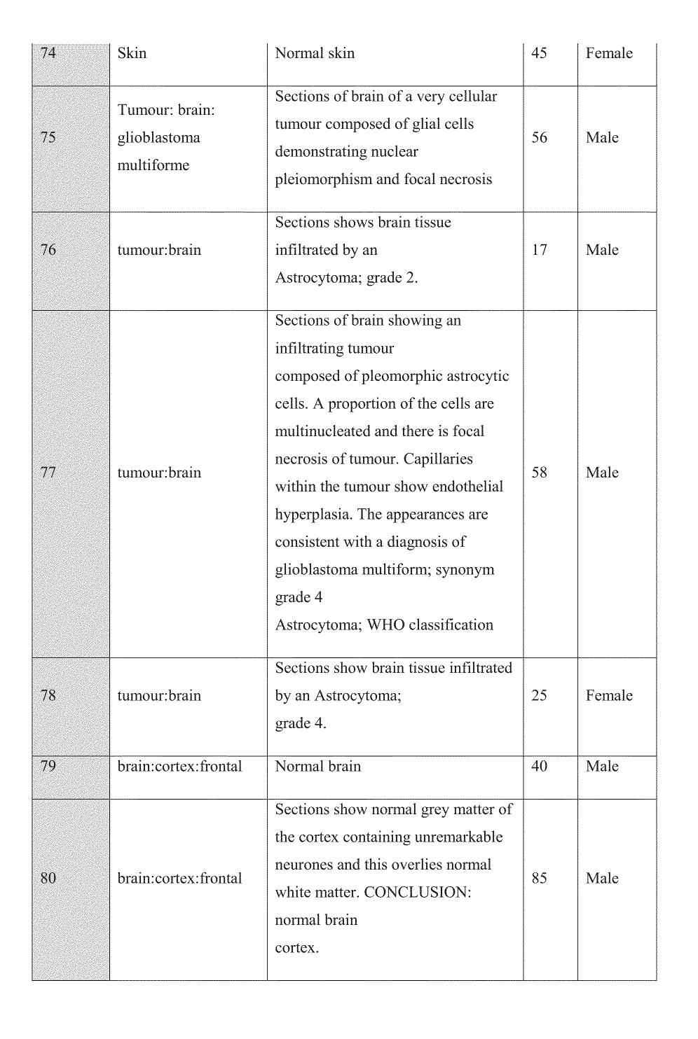

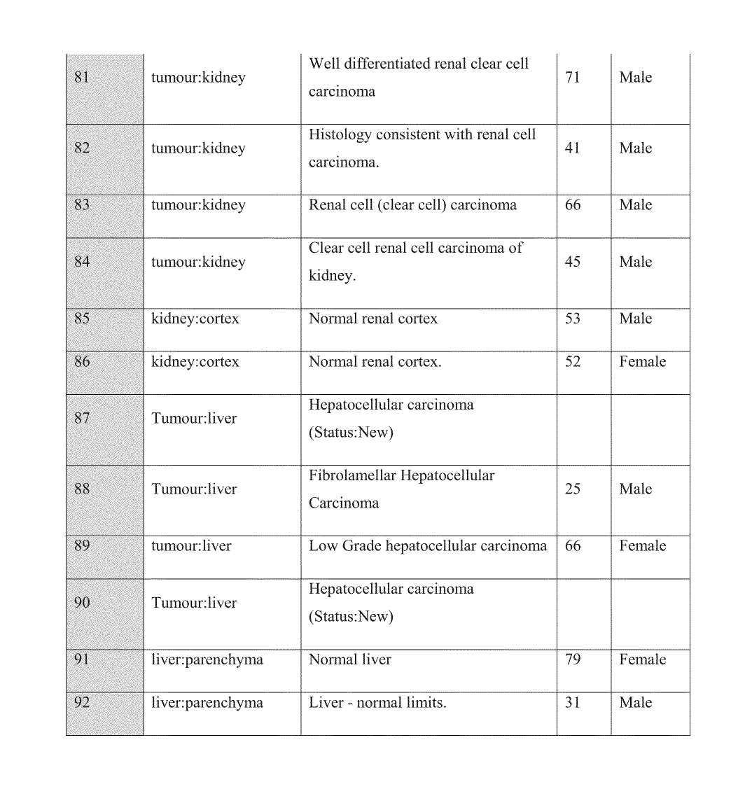

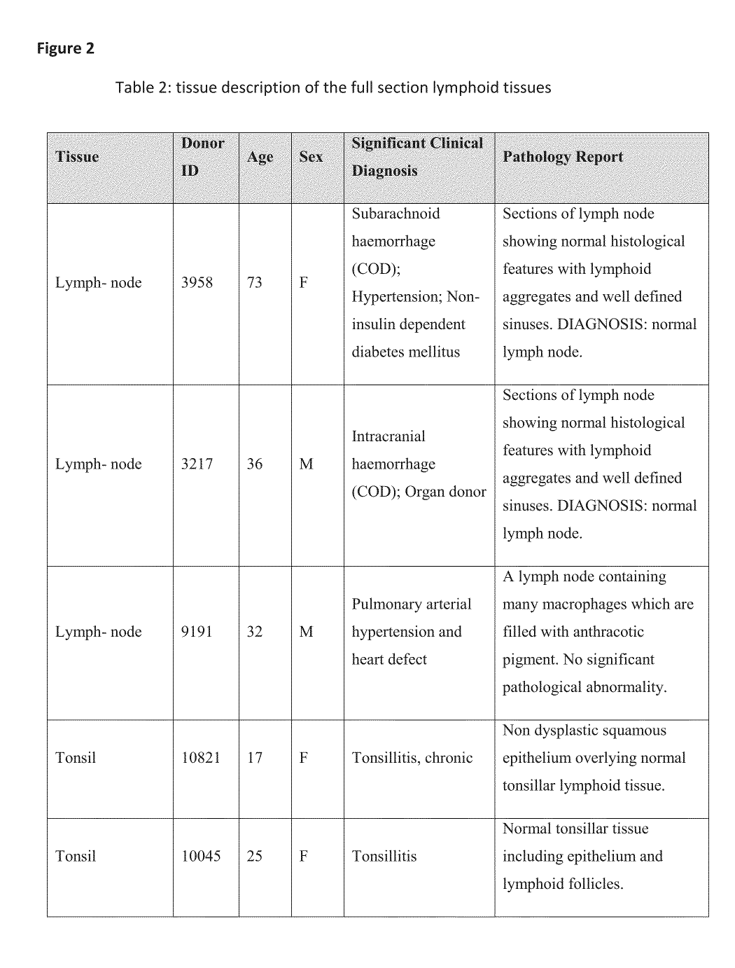

Further additional analysis in normal tissue sections of lymph node (n=3), tonsil (n=3) and spleen (n=3) were included as described in FIG. 2. Both FIG. 1 and FIG. 2 present full clinical details of the samples used. As described therein FFPE sections (4 μm) of the cell line HEK293T which recombinantly expression VSTM5-GFP, the ‘multi-tumor’ TMA and full-face sections of normal lymph node, tonsil and spleen were used.

Tissue Details: TOP4 TMA

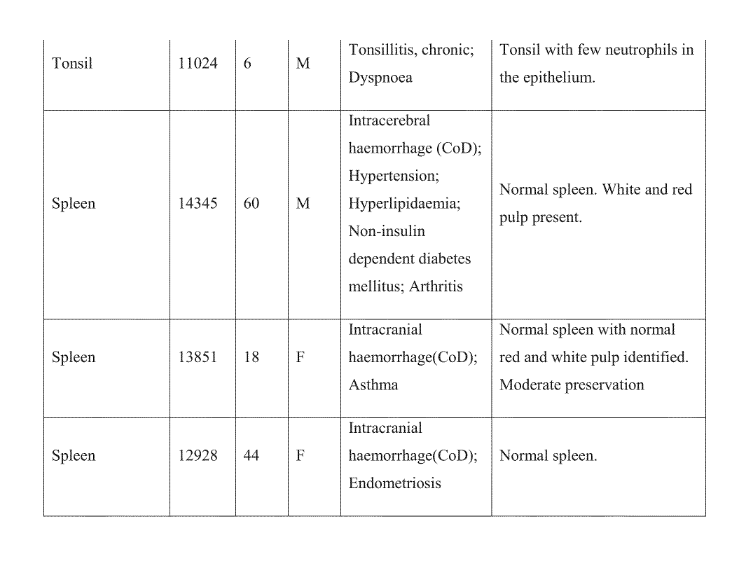

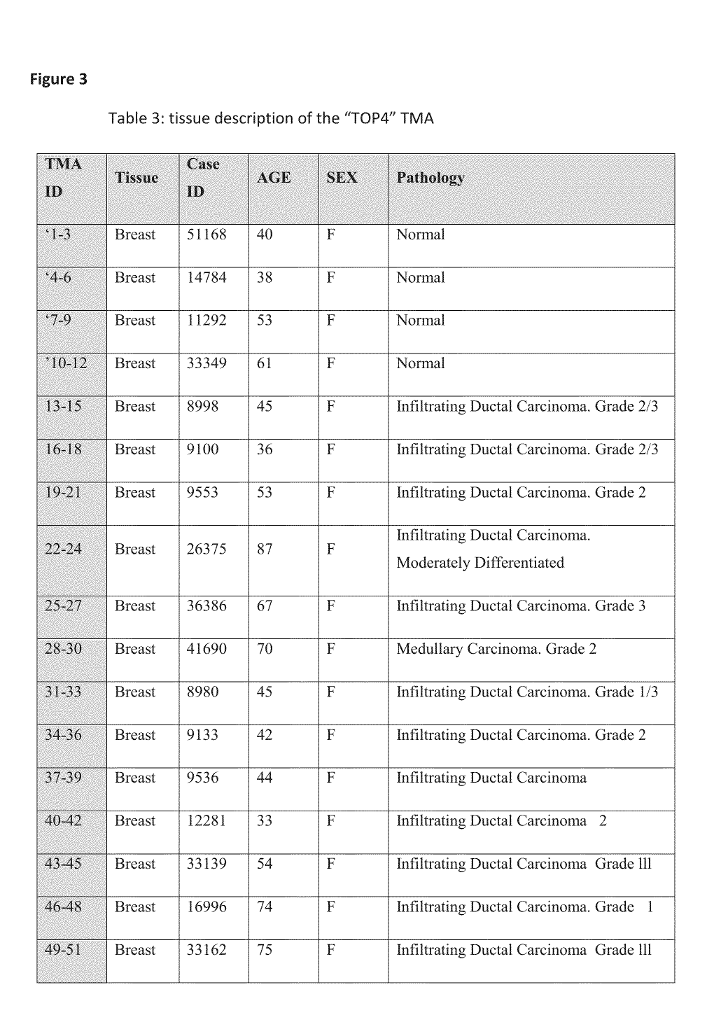

As described herein the ‘Top 4’ TMA is comprised of triplicate tissue cores (0.6 mm diameter) from a total of 120 different donors with an age range of 25-89 years, of which 49 were female and 71 were male. The TMA consisted of cores from 4 tissue types: breast (4 normal and 26 tumors), large intestine (4 normal and 26 tumors), lung (4 normal and 26 tumors) and prostate (4 normal and 26 tumors) FIG. 3 presents full description of the “Top 4” tissue microarray samples used.

Antigen Retrieval and Staining

The sections were de-paraffinized; antigen retrieved and rehydrated using pH9.0 Flex⁺3-in-1 antigen retrieval buffers, in PT Link apparatus at 95° C. for 20 min with automatic heating and cooling.

Following antigen retrieval, sections were washed in Flex (TBST) buffer for 2×5 min then loaded into a DAKO Autostainer Plus. The sections were then incubated for 10 min with Flex+ Peroxidase Blocking reagent, rinsed twice in 50 mM Tris. HCl, 150 mM NaCl, 0.1% Tween-20, pH 7.6 (TBST), followed by a 10 min incubation with Protein Block reagent (DAKO X0909).

The sections were incubated for 30 min with primary antibody diluted in DAKO Envision Flex antibody diluent (DAKO Cytomation, Cat # K8006).

Anti VSTM5 (C110rf90) (HPA029525 Sigma) was applied at 3 μg/ml. Anti Von Willebrand's Factor (vWF) antibody was applied at 1 ug/ml. The negative control sections were incubated with non-immune rabbit IgG antibodies (Dako, CAT #0936) at 3 and 1 μg/ml or in DAKO Envision Flex antibody diluent (‘no primary’ control).

Following incubation with primary antibodies, the sections were then rinsed twice in FLEX buffer, incubated with anti-mouse/rabbit Flex+HRP for 20 min, rinsed twice in FLEX buffer and then incubated with diaminobenzidine (DAB) substrate for 10 min. The chromogenic reaction was stopped by rinsing the slides with distilled water.

Following chromagenesis, the sections were counterstained with haematoxylin, dehydrated in an ascending series of ethanols (90-99-100%), and cleared in three changes of xylene and coverslipped under DePeX.

Results

The sections were analyzed for the intensity of the specific staining and a semi-quantitative scoring system was used. The core in the tissue array with the most intense VSTM5-ir was assigned a score of 3+ and the intensities of the immunoreactivity in the other cores were scored relative to that of the 3+ core. The percentage of VSTM5-ir tumor was estimated and recorded using the following ranges: 0-25%, 25-50%, 50-75% and 75-100%. Where scoring was too low to quantitate—an assigned ‘+’ was used to denote the presence of staining. The intracellular distribution of the immune-stained cells in the tumor was also recorded.

Result Summary of the ‘Multi-Tumor’ TMA

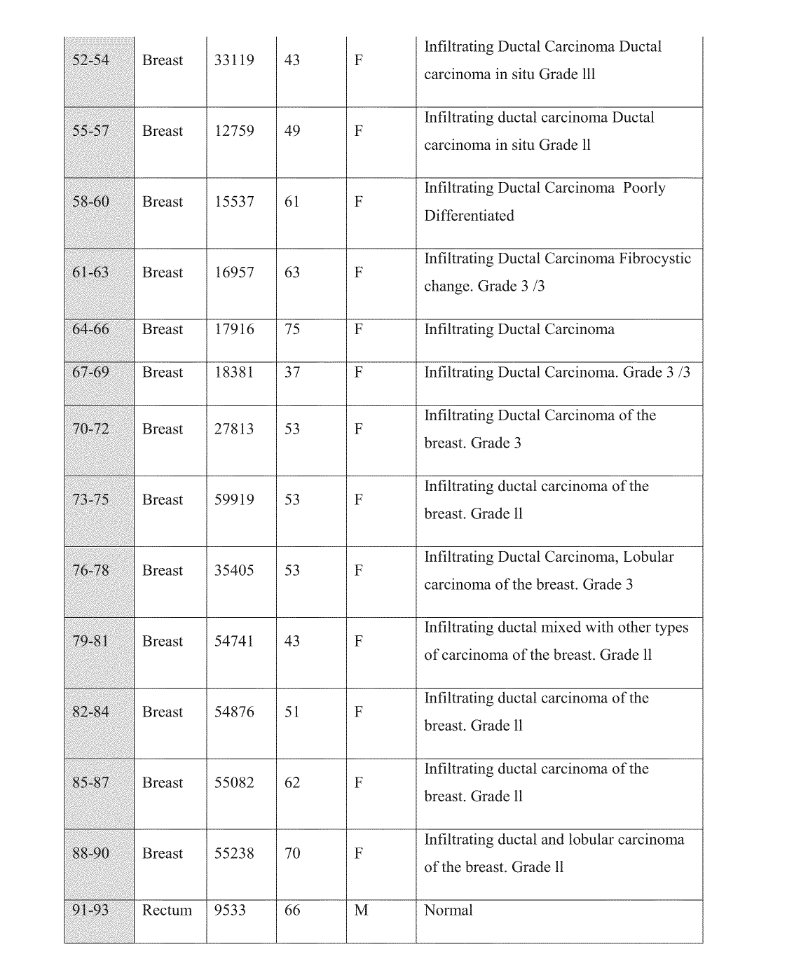

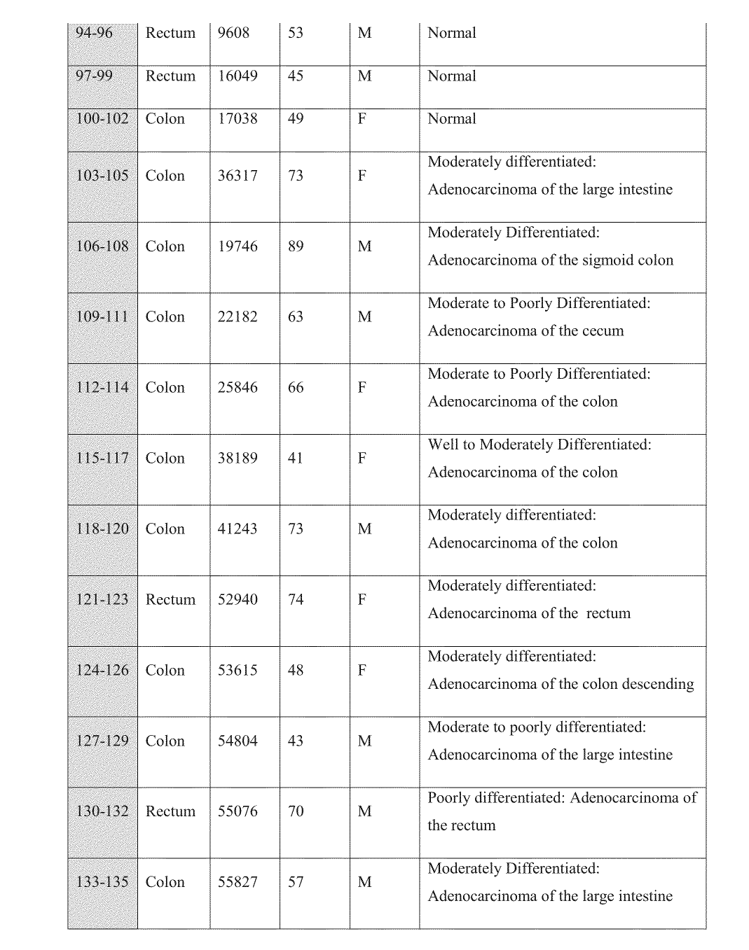

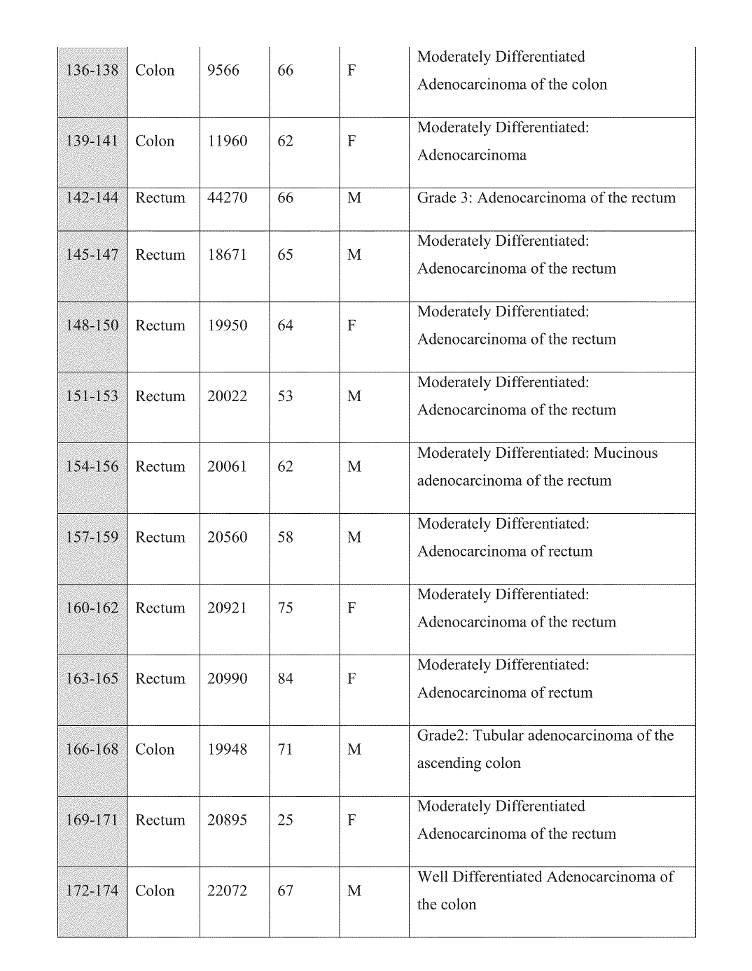

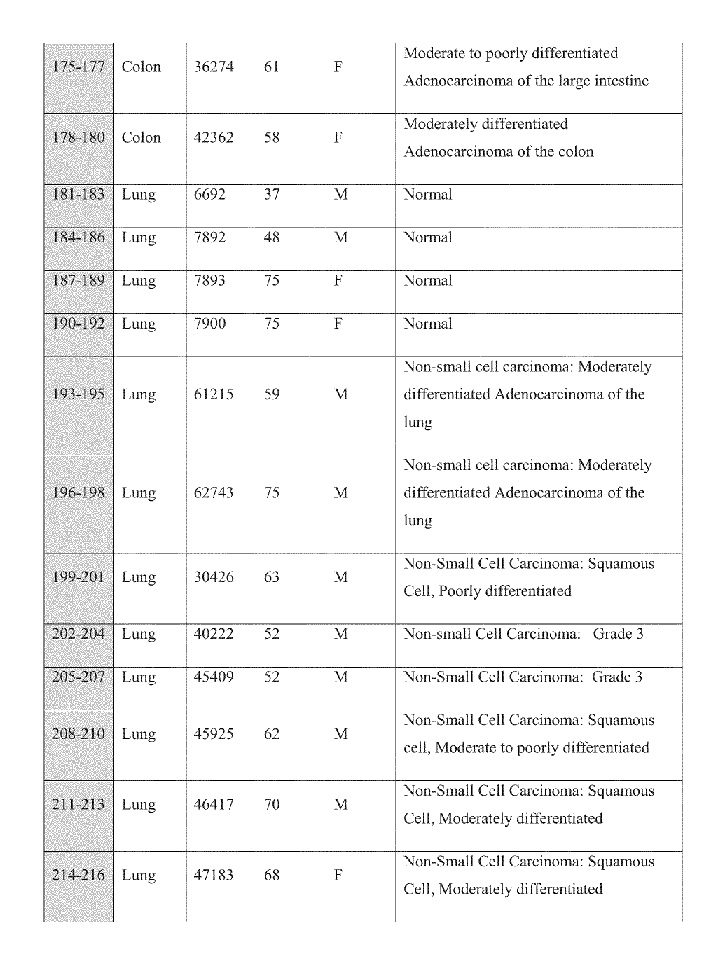

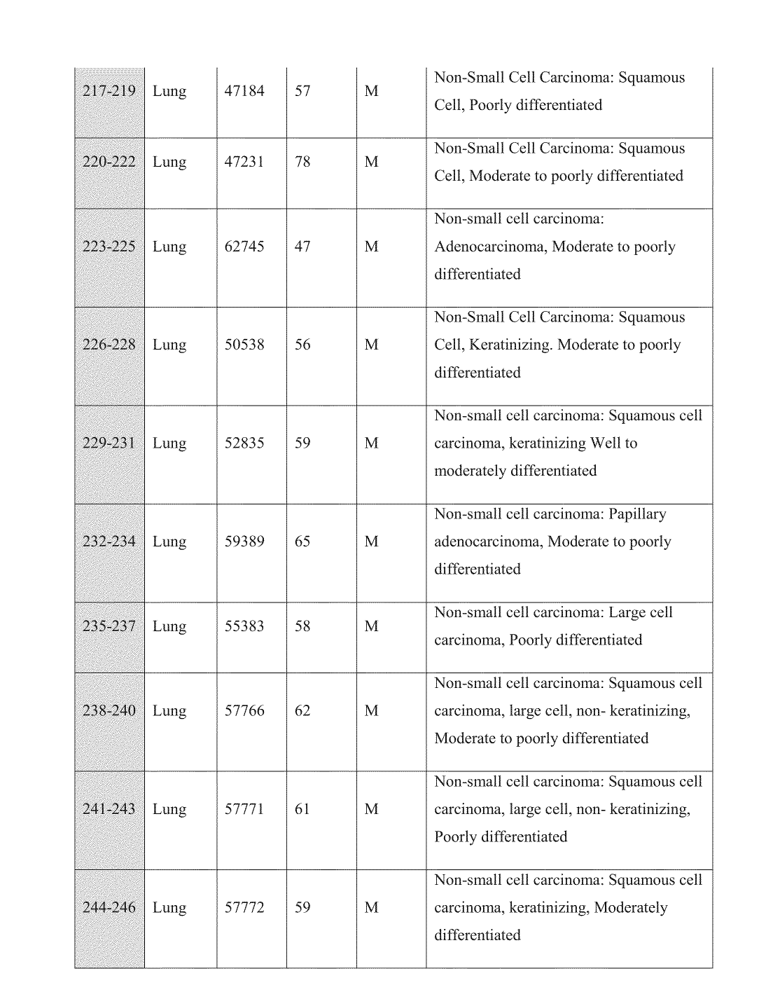

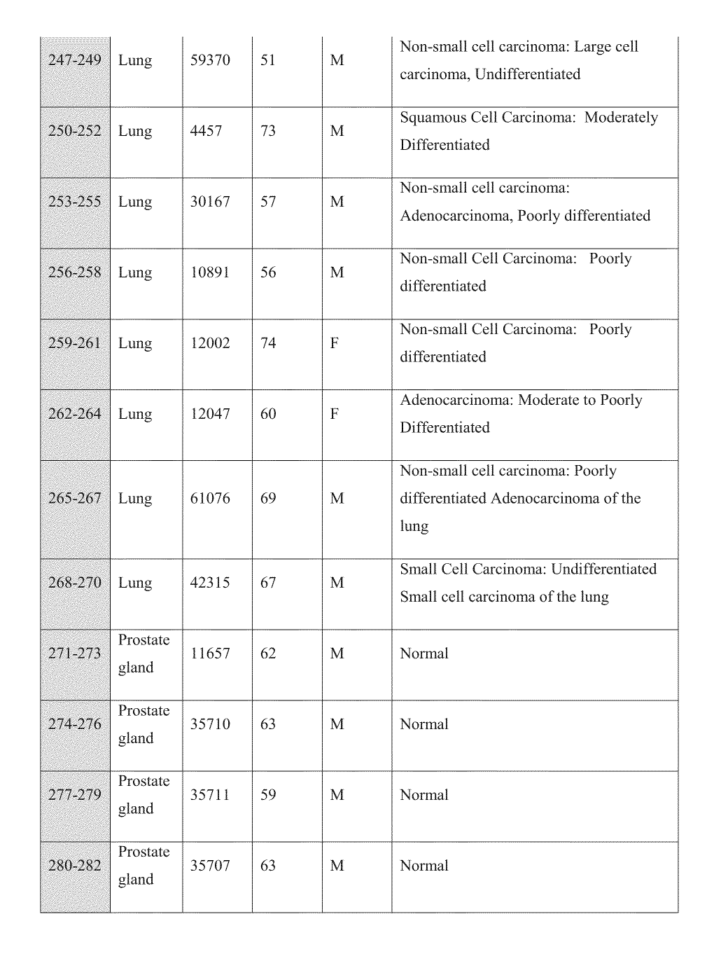

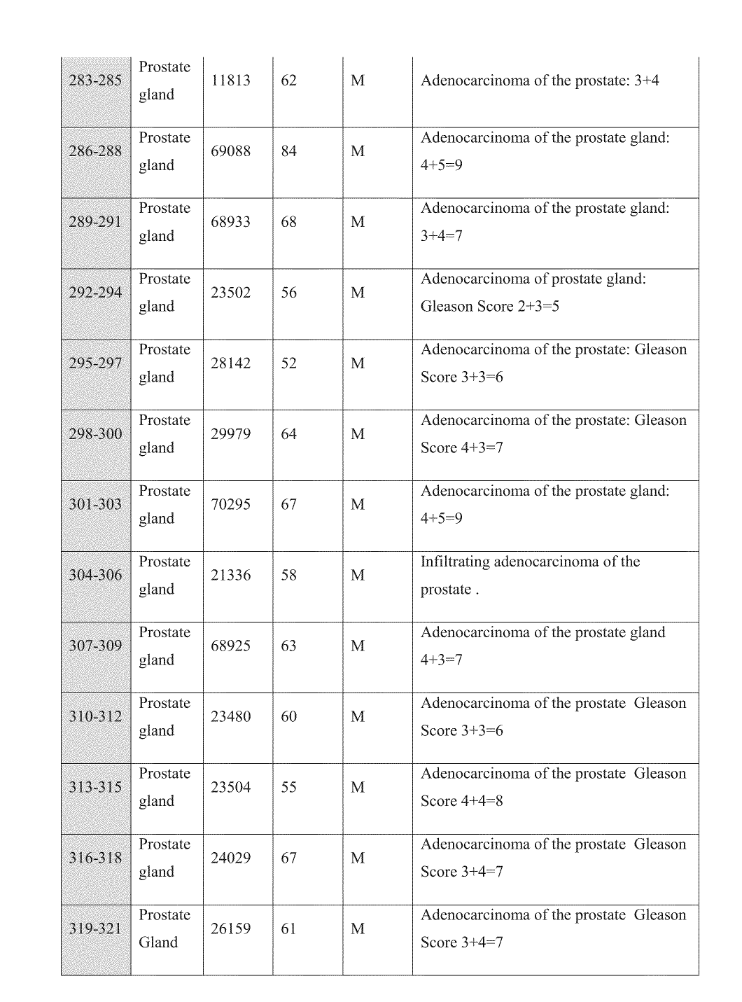

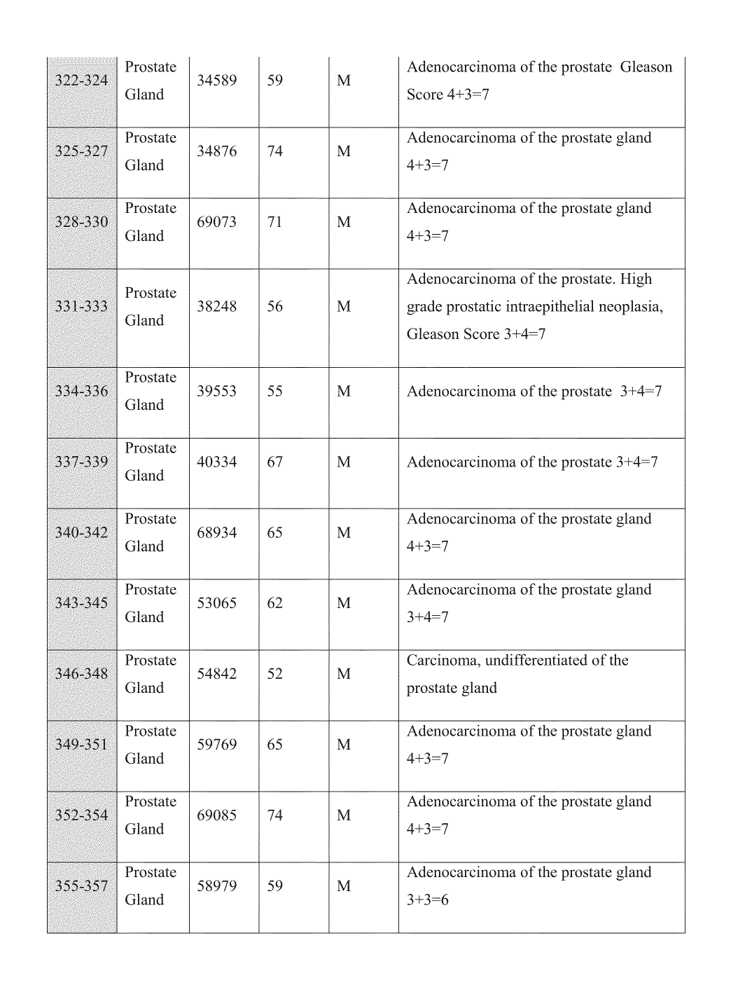

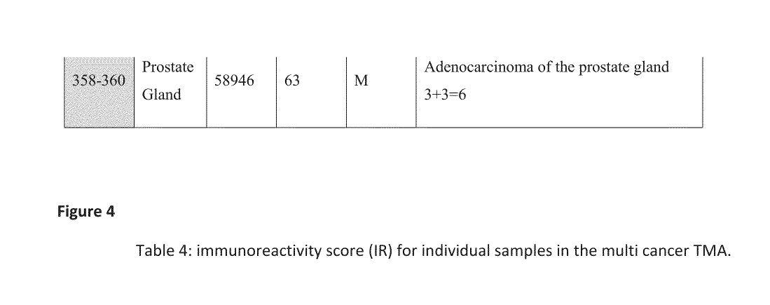

The following observations can be made upon review of the summary of IHC scores of individual samples in FIG. 4:

Within the breast tumor cohort, the majority of staining seen was weak to moderate, with 50-100% of tumor cells being immunoreactive. In one sample, occasional immune cells were also observed to show intense staining. In normal breast, no apparent immunoreactivity was observed within the tissue, with exception of a few infiltrating immune cells within the lobular acini regions.

Within the large bowel cohort, staining intensity was seen to be moderate to high within the tumor epithelium, within 75-100% of tumor cells immunoreactive. It was noted that three donor samples were also seen to exhibit intense cytoplasmic-membrane staining in discrete tumor cells. Within the stroma, immunoreactivity was also present and restricted to highly-stained putative immune cells. In the normal tissue set, specific diffuse-cytoplasmic immunoreactivity was seen in the mucosal epithelium and putative immune cells.

In prostate tumors, all but one sample appeared to be immunoreactive in 75-100% of tumor cells. In these cores, the staining pattern and intensity was weak In one sample, a few discrete tumor cells were shown to be highly stained, and where present—other staining observations were seen in immune cell infiltrates. In the normal prostate samples, specific weak-cytoplasmic immunoreactivity was detected in the glandular epithelium, and in putative fibroblasts.

In lymphoma samples, all donors were shown to be immunoreactive in 75-100% of tumor cells. In general, the pattern and intensity appeared to be weak throughout. Occasional tumor cells were also shown to demonstrate intense (3+) staining. In normal lymph node, specific cytoplasmic immunoreactivity was detected in lymphocytes within the cortex and germinal centers.

In the lung tumor set, eleven out of twelve samples demonstrated specific immunoreactivity within 75-100% of tumors. In non-small cell (NSCLC) tumors, weak immunoreactivity was seen in two samples. In two squamous tumor samples, immunoreactivity was seen in tumor islands, scoring (1+) in a moderate-poorly differentiated sample, with highly-stained infiltrating immune cells. In a moderate-well differentiated sample, a (3+) staining was seen. In adenocarcinoma, three samples scoring staining of (1+) is observed in poorly differentiated tumors. In one notable adenocarcinoma sample, 2+ staining was seen in tumors, with occasional highly stained infiltrating immune cells. In one small-cell carcinoma sample, 2+ staining was seen. In the normal lung samples, specific cytoplasmic immunoreactivity was seen in the respiratory epithelium, with highly-stained luminal surfaces, free alveolar macrophages and occasional putative fibroblasts.

In stomach tumors, four moderately differentiated adenocarcinoma samples demonstrated specific immunoreactivity in 75-100% of the tumor cells. The intensity of staining seen varied between donors, ranging from (0-1), (1-2) and (3+) in tumor cells. Highly-intense staining was seen in discrete tumor cells. Infiltrating immune cells were also seen to be immunoreactive, and highly stained in these samples. In normal stomach tissue, apparently specific ‘intense’ membrane-immunoreactivity was seen in the superficial mucosal epithelium, and diffuse-cytoplasmic staining was seen in the sub mucosa, with occasional ‘intense’ staining of discrete cells.

In the ovarian carcinoma cohort, specific immunoreactivity was seen in 75-100% of tumor cells. In the cystadenocarcinoma samples, moderate to intense granular staining was seen in tumor cells—ranging (2+ to 3+). Occasional intense staining of infiltrating immune cells was also noted in these samples.

In one granulosa sample, weak immunoreactivity was seen in tumor cells, staining (1+). In the normal ovarian samples, only one donor, showed specific nuclear-cytoplasmic immunoreactivity in discrete stromal cells. The other ovary sample was wholly negative.

In skin melanoma, weak immunoreactivity was generally seen in the tumor samples. Infiltrating immune cells were also immunoreactive in these samples, and appeared to be intensely stained. In normal skin, apparently intense (3+) cytoplasmic staining was seen in the epidermis from two donors. No other notable immunoreactivity was detected in these samples.

In brain tumor, no apparent immunoreactivity was detected in the majority of donors. In one sample, only a few tumor cells were seen to be immunoreactive. In normal brain, no immunoreactivity was seen.

In the renal carcinomas, two of the three clear-cell type tumors demonstrated immunoreactivity within 75-100% of cells, staining (1+) and (2+) in each core respectively. Other staining features were seen in occasional putative infiltrating immune cells in cores. In the normal kidney samples, a weak diffuse-cytoplasmic staining was generally seen in the collecting tubular epithelium. Intense cytoplasmic staining was also noted in a few cells of the proximal convoluted tubules.

In liver carcinomas, three samples demonstrated weak to moderate immunoreactivity in tumor cells, where 75-100% were stained (1+, 2, 2+) respectively in each core. One particular donor, was seen to have occasional intense (3+) staining in tumor cells. In normal liver samples, a very weak-cytoplasmic blush staining was seen in, and restricted to normal hepatocytes and putative immune cells.

In full-face sections of normal lymph node, tonsil and spleen, it was observed that the majority of samples demonstrated specific staining in immune cells within the germinal centers/paracortex from the three tissue sets. In lymph node, cytoplasmic heterogeneity was seen throughout the tissue samples. In tonsil, specific cytoplasmic staining was seen in the germinal centers and of the paracortex, notable immunoreactivity was also seen in the squamous epithelium of one donor. In spleen samples, specific immunoreactivity was detected in occasional immune cells of the germinal centers, and pulp regions.

Results

Result Summary ‘TOP4’ TMA

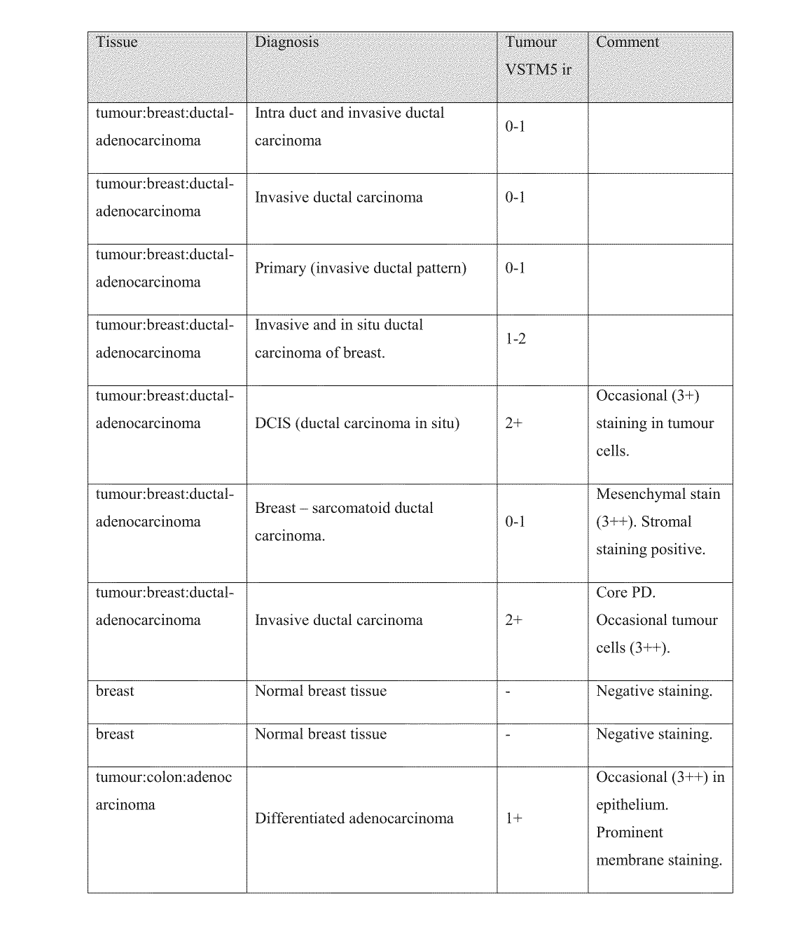

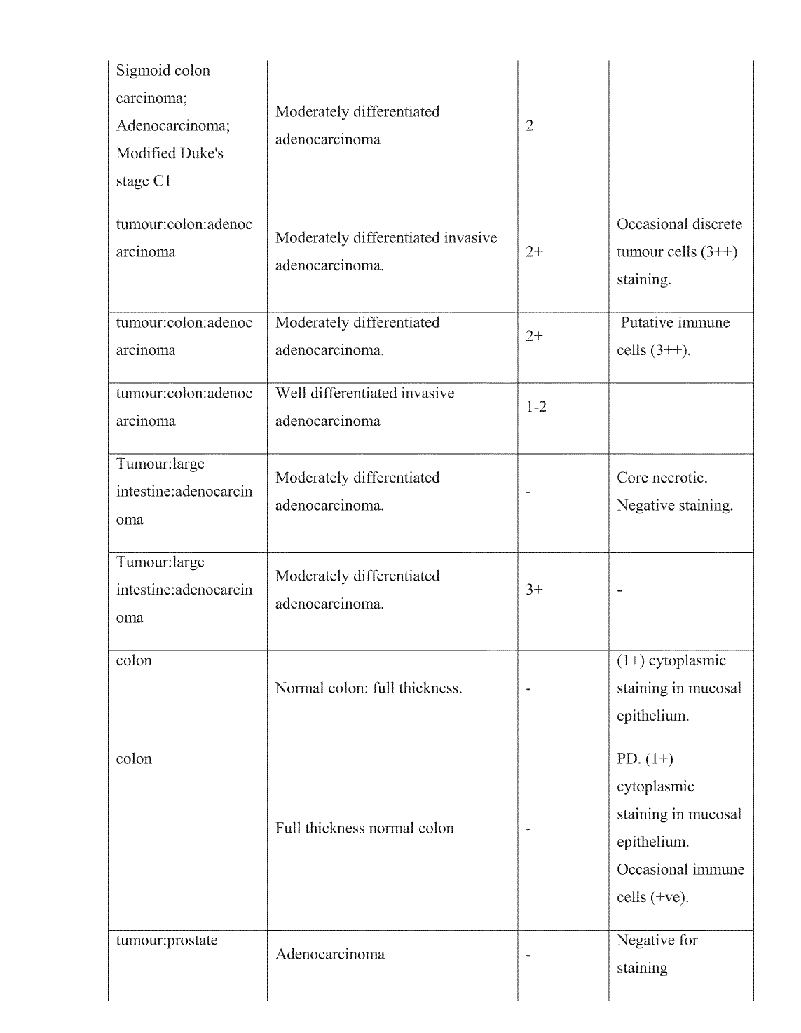

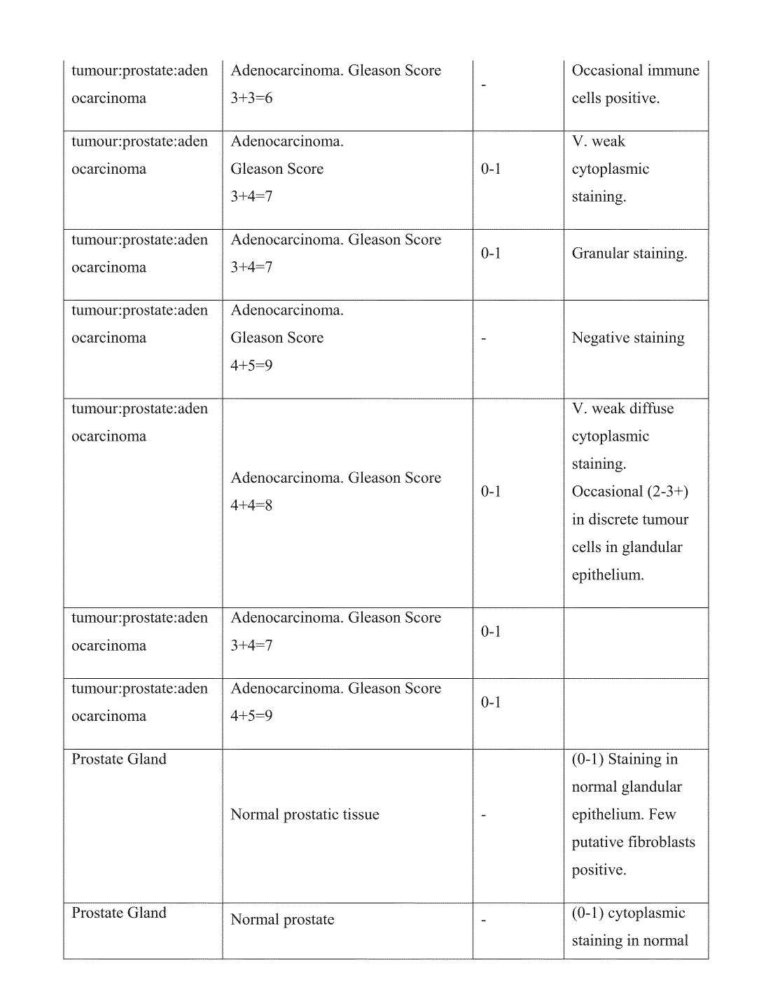

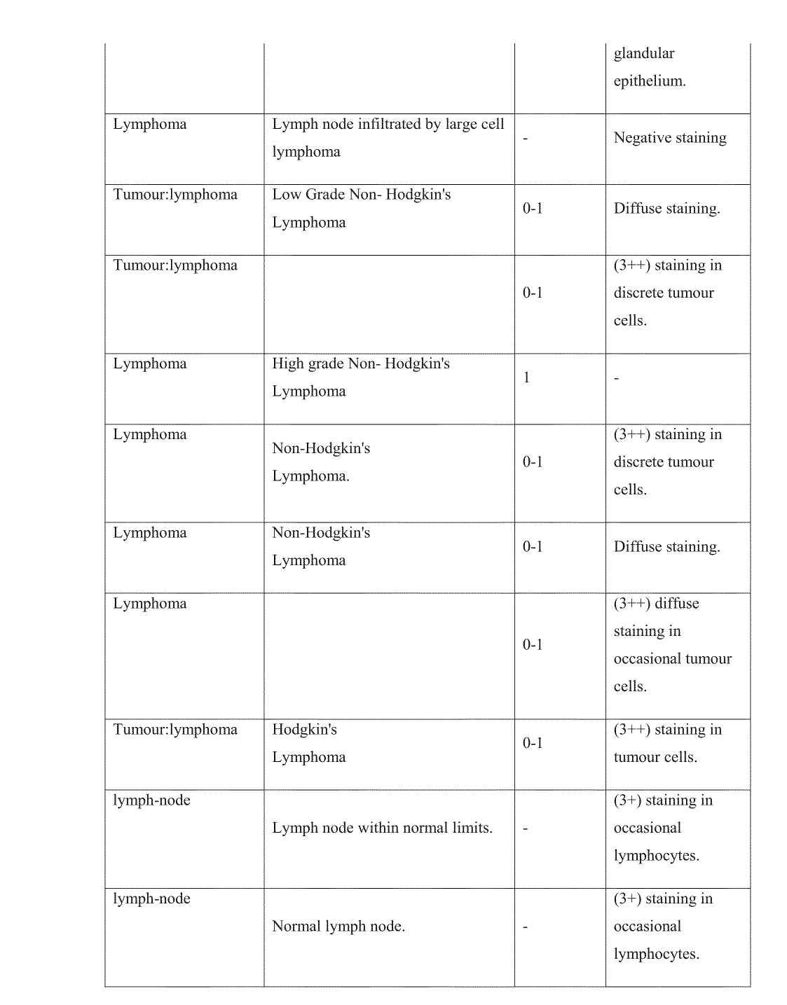

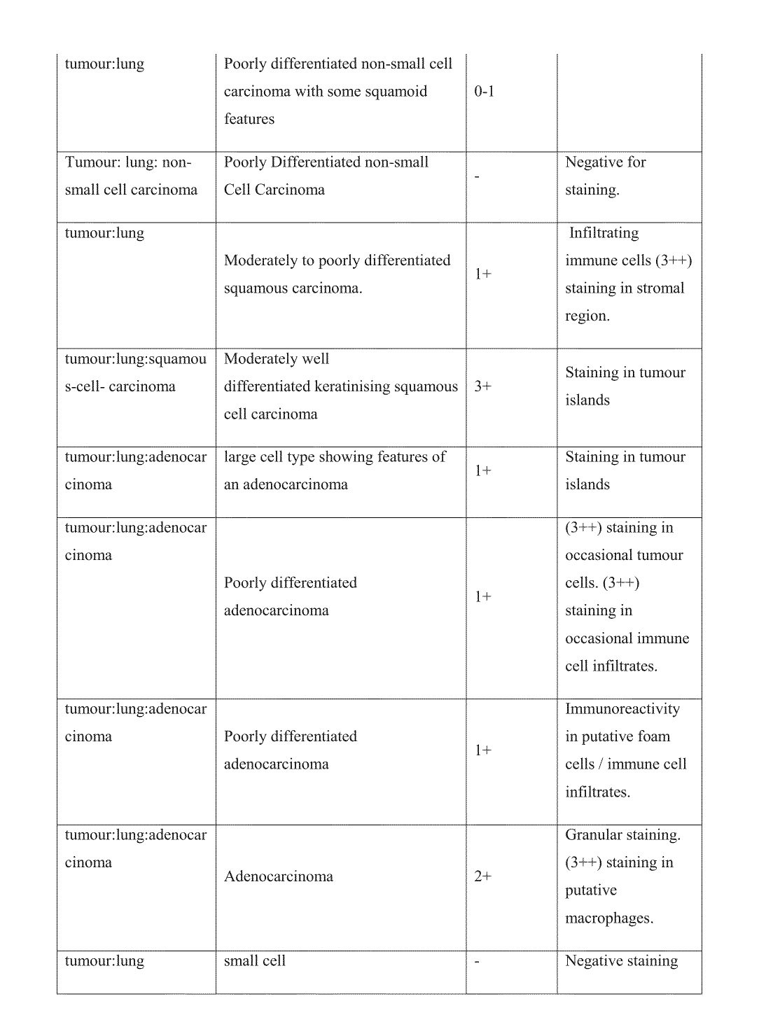

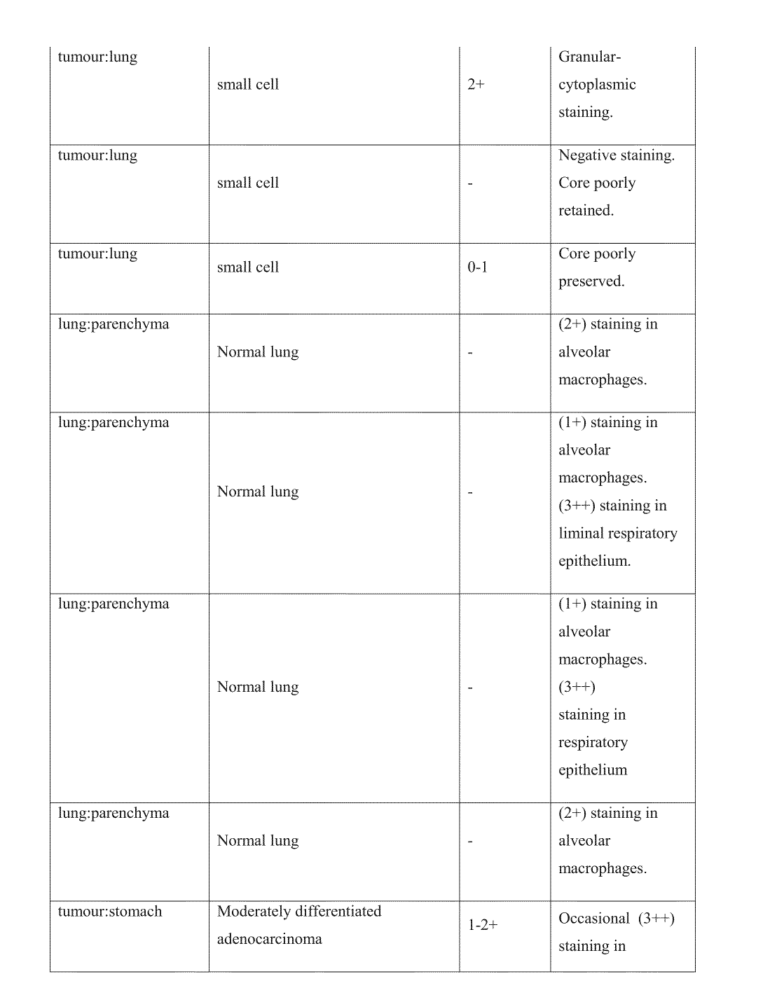

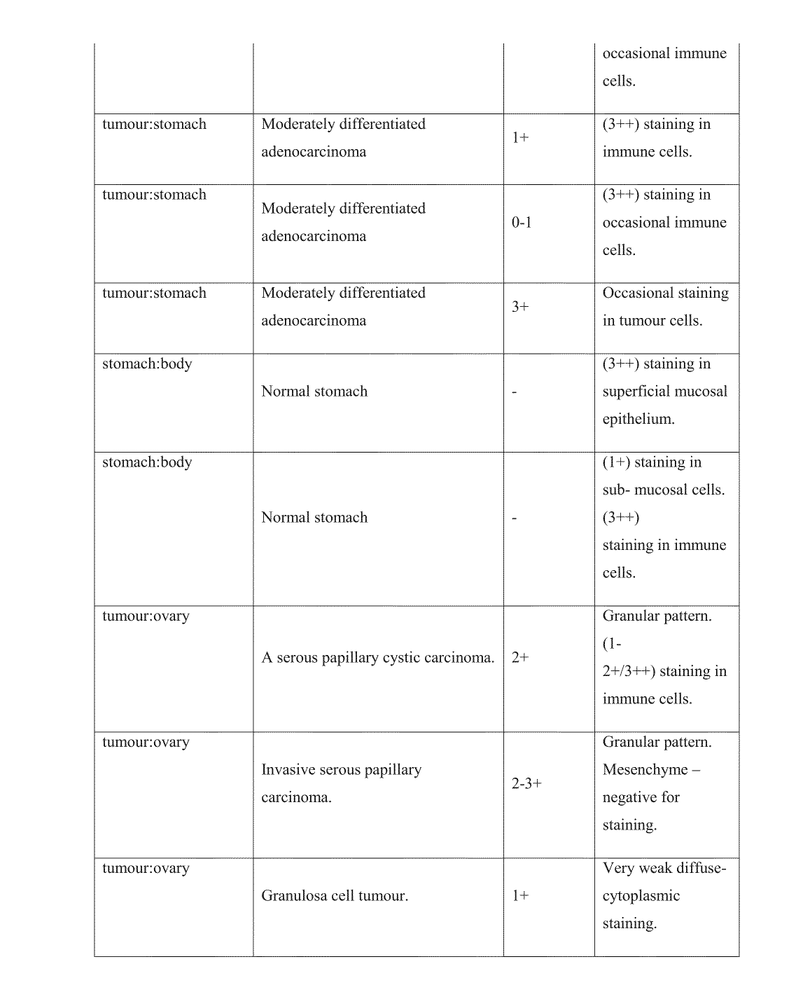

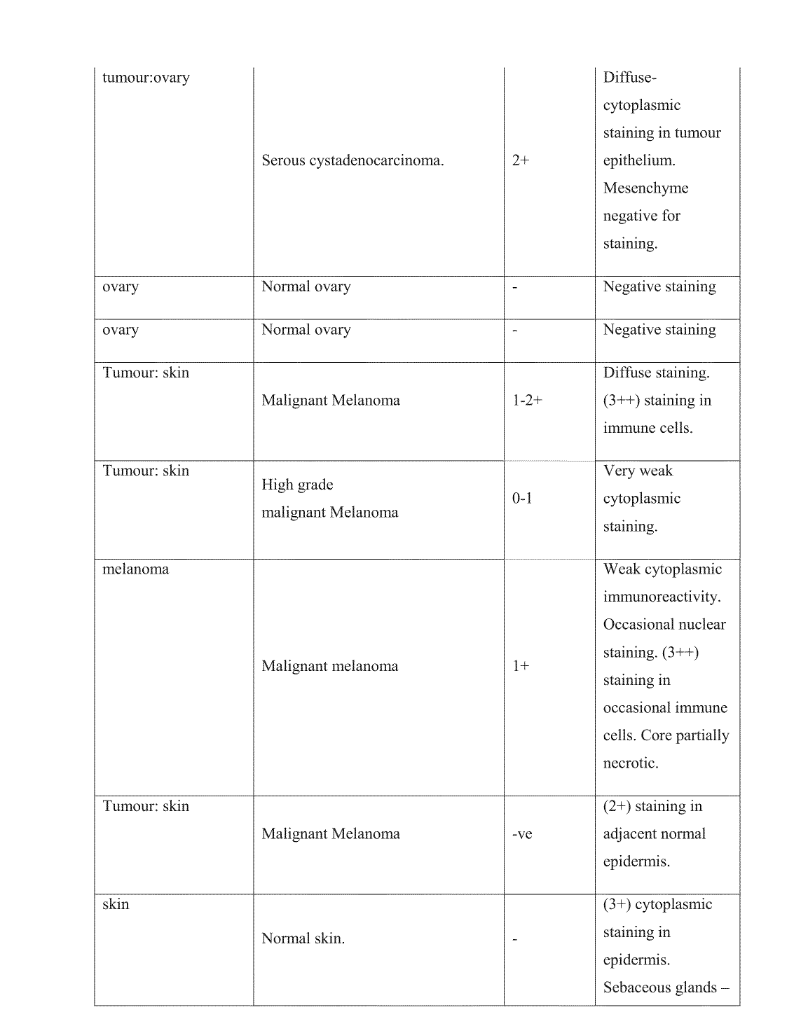

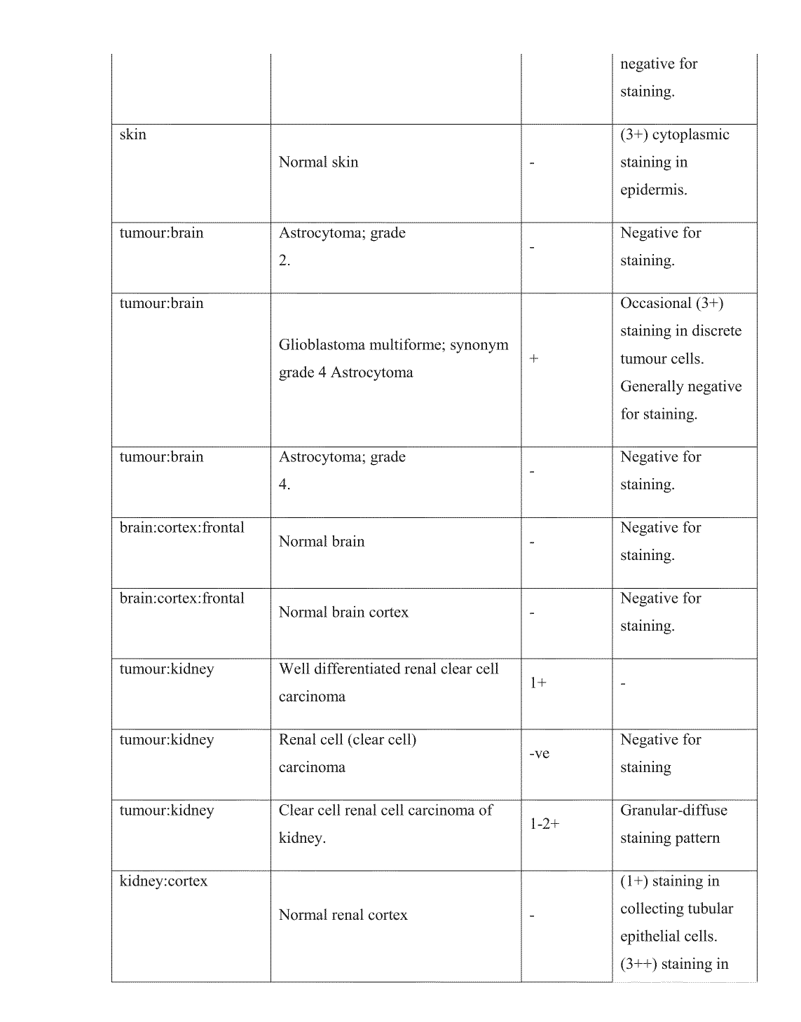

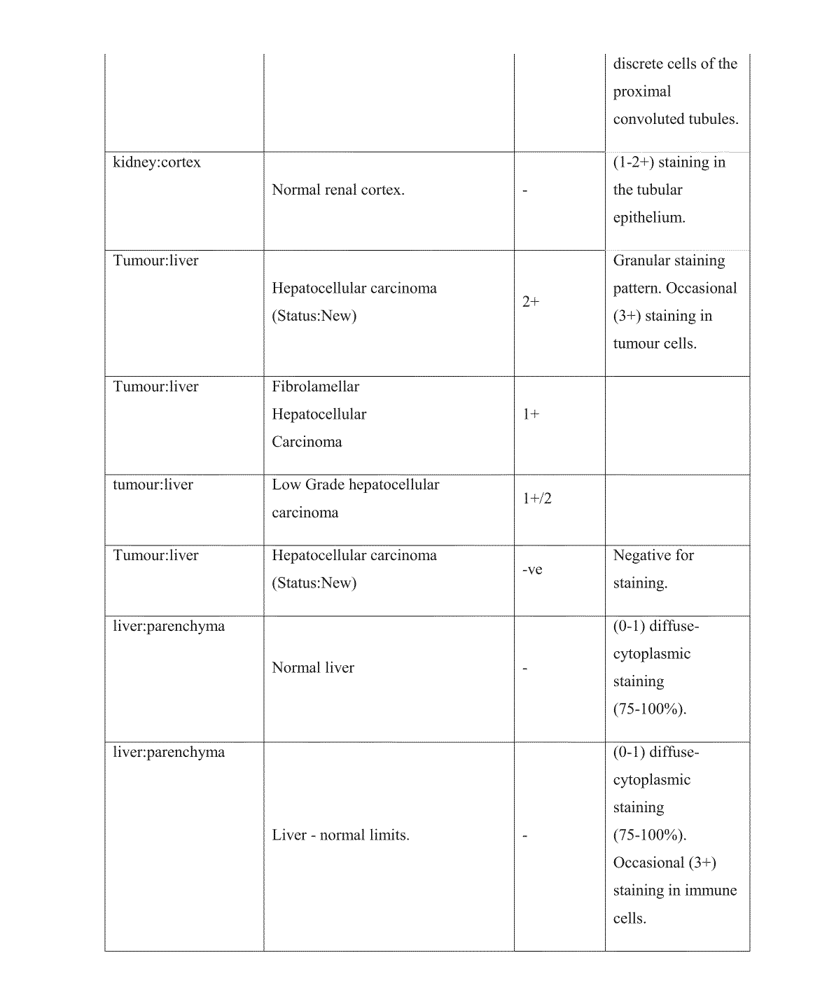

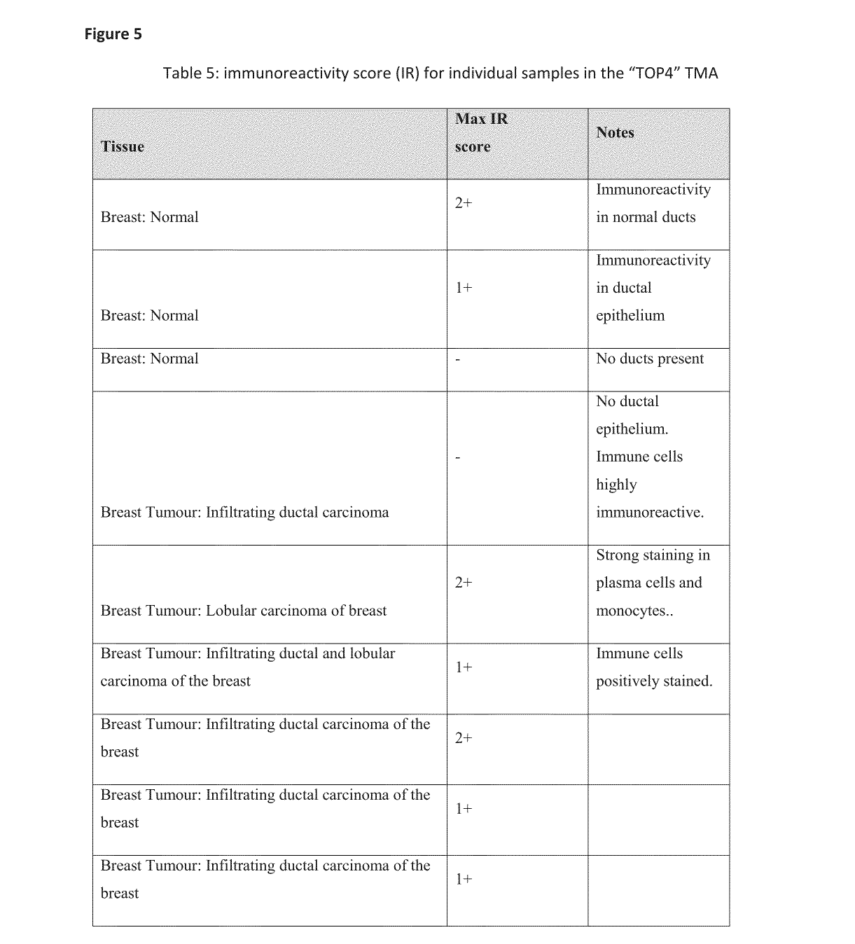

The following observations can be made upon review of the summary of IHC scores of TOP4 TMA samples in FIG. 5:

Within the breast tumor set, the intensity of staining seen was weak to moderate in majority of cases. In this cohort, two samples scored a maximum intensity of 3-3+ within 50-100% of reactive tumors, tumor grades 2/3. Within five of the samples, the staining intensity scored a maximum of 2+, within 50-100% of tumors (grades 2/3). Thirteen other samples scored a lower intensity of +1 of which most tumor samples were 25-100% reactive. Within the stromal regions, infiltrating immune cells were seen highly stained—notably in putative monocytes and plasma cells. The majority of tumors were mainly infiltrating ductal and lobular carcinomas—mixed grades. In normal breast, weak/moderate immunoreactivity was seen within the glandular acini.

Within the large bowel cohort, the adenocarcinoma samples were moderate to well differentiated types. In four samples, an assigned score of 3+ staining was seen within 50-100% of tumor cells of tumor grades 2/3. In fourteen other samples, a score of 2+ was seen in 50-100% of cells from reactive tumor grades 2/3. In the last four samples, a weaker score of 1+ was seen in tumors of moderate-poorly differentiated cell types. Other immunoreactive regions include highly stained infiltrating immune cells. In normal tissue samples, specific immunoreactivity was detected in mucosal epithelium and resident inflammatory cells.

In the lung tumor set, specific immunoreactivity was seen in the majority of tumors investigated, where a weak to moderate staining intensity was noted. The majority of tumors were non-small cell lung carcinomas (NSCLC)—of adenocarcinoma origin, of moderate to poorly differentiated cell types. In these tumors, two samples were assigned a maximum intensity score of 2+, of which 25-100% of tumor cells were immunoreactive. In seventeen other samples, a weaker score of 1+ was seen in 25-100% of tumors. In one sample of small-cell carcinoma immunoreactivity was weak (1+), within 25-50% of tumor cells. Highly immunoreactive infiltrating immune cells were prominent. Pathological scores indicate a heterogeneous pattern of staining of the same tumor type(s).

In the normal lung tissue, immunoreactivity was detected in one sample of bronchial epithelium. No apparent immunoreactivity was detected in other normal lung cores. Occasional free-macrophages were only seen to be immunoreactive.

In the set of prostate tumors, specific staining was seen in most samples, where intensity of staining was weak to moderate in the tumor epithelium. In four samples, a maximum assigned score of (3+) staining was seen in 50-100% of tumors, (Gleason scores 3+3, 4+3 and two—3+4 samples). Eleven other samples had a score of 2+ within 50-100% of tumors. Most of the staining was in tumor islands, with Gleason scores ranging from (3+4), (4+5) and (4+3) respectively. Lastly, six tumor core samples were scored a weaker 1+ staining in 25-100% of tumors. In the normal prostate tissues, a few samples demonstrated weak-moderate cytoplasmic staining in the glandular epithelium. Other notable staining was seen in putative infiltrating immune cells and fibromuscular regions.

Overall, the results of the TMA and TPP4 samples, which comprise a wide variety of different human tumor samples, and which are derived from different types of tumor tissues and lymphoid tissues indicate that VSTM5 protein is expressed in a large proportion of tumor types. Moreover, in such tumor types, including tumors with relatively low immunoreactivity, immune infiltrating cells were positive, further supporting the immune modulatory role of VSTM5 protein. These results coupled with the functional data in the examples which follow corroborate the fact that VSTM5 binding agents, e.g., immunomodulatory anti-VSTM5 antibodies or antigen-binding fragments should suppress or potentiate the effects of VSTM5 in different human disease conditions, e.g., cancer or infectious disease, wherein the expression of VSTM5 seems to have a suppressive effect on the subject's antitumor immune response.

### Example 2

### Generation and Characterization of VSTM5-Expressing Stable Transfectant Cell Pools

As described herein, in these experiments we generated recombinant stable pools of cell lines overexpressing VSTM5 human and mouse proteins, for use in determining the effects of VSTM5 on immunity, for VSTM5 characterization, anti-VSTM5 antibody discovery, and to obtain cross-species anti-VSTM5 antibodies.

Materials & Methods

Expression Constructs

The coding sequence in each of the various expression constructs used in this example was obtained either by full length cloning using RT-PCR derived cDNAs or by gene synthesis, followed by subcloning to mammalian expression vectors.

Full length cDNA of human VSTM5 (SEQ ID NO:4) was obtained by RT-PCR using lung cancer cDNA as a template with gene specific primers, as described in s described in PCT/US2008/075122, owned in common with the present application. The full length cDNA was subsequently cloned into expression vectors to create the constructs described below. All cDNA inserts were digested with specific restriction enzymes and ligated to pIRESpuro3 (pRp3) mammalian expression vector (Clontech, Cat No: 631619) previously digested with the same enzymes.

Construct Encoding Human VSTM5-Untagged

The full length cDNA of human VSTM5 (SEQ ID NO:4) was cloned in the pIRESpuro3 (pRp3) mammalian expression vector, as described in PCT/US2008/075122, owned in common with the present application.

Construct Encoding Human VSTM5-EGFP

Full length cDNA of human VSTM5 (SEQ ID NO:4) was cloned in frame to the N terminus of EGFP in EGFP-pIRESpuro3 (Chen et al., Molecular vision 2002; 8; 372-388) for expression of a VSTM5-EGFP fusion protein. Subcloning was performed by PCR using the above human VSTM5 (SEQ ID NO:4)-untagged expression vector as template.

Construct Encoding the Fusion Protein Human VSTM5 ECD mIgG2a (SEQ ID NO:10)

Cloning of the fusion protein of the extracellular domain (ECD) of VSTM5 fused to mouse IgG2a Fc, was carried out in two steps: first, cloning of ECD to pIRESpuro3; and second, subcloning of the mouse IgG2a Fc in frame to the C′ terminus of the ECD previously cloned into pIRESpuro3, from step one. Cloning of the ECD was done by PCR using VSTM5 full length sequence as a template, as described in PCT/US2008/075122, owned in common with the present application. The resulting expression constructs were verified by sequence and subsequently used for transfections and stable pool generation as described below.

Construct Encoding Mouse VSTM5 (SEQ ID NO:11)

Full length cDNA encoding mouse-VSTM5 protein (SEQ ID NO:11) was synthesized, and cloned in pUC57 vector at GeneScript. This cDNA was subsequently cloned in the pIRESpuro3 mammalian expression vector as described below. The resulting expression constructs were verified by sequence and subsequently used for transfections and stable pool generation as described below.

Generation of Stable Transfectant Pools Expressing Mouse VSTM5, Human VSTM5 or Human VSTM5-EGFP Proteins

The expression constructs described above were used to generate stable pools of VSTM5 expressing HEK293T cells by DNA transfection, followed by establishment of resistant pools of colonies with the specific selection media. Each of the parental cell lines was also transfected with an empty vector, used as negative control.

HEK-293T (ATCC, CRL-11268) cells were transfected with the mouse, human VSTM5 and human VSTM5-EGFP pRp3 constructs described above or with the empty vector (pRp3) as negative control, using Fugene6 transfection reagent (Roche, Cat No: 11-988-387). Puromycin resistant colonies were selected for stable pool generation.

Expression Validation

Whole cell extracts of each cell pool (30 ug of total protein) were analyzed by western blot. As negative control, whole cell extracts of stable cell pools transfected with the empty vector were used. A commercial rabbit anti-VSTM5 polyclonal Ab diluted 1:100 was used (Sigma, Cat. No HPA029525), followed by secondary goat anti-rabbit conjugated to HRP (Jackson, 111-035-003) Ab diluted 1:10000 in blocking solution.

In order to validate the cell surface expression of VSTM5 (SEQ ID NO:132) or VSTM5-EGFP (SEQ ID NO:1331 proteins in the recombinant stable pools, 1×10⁵ cells were stained with 10 ug/ml of commercial anti VSTM5 rabbit pAb (Sigma, Cat. No HPA029525) or rabbit IgG control (Sigma, Cat No 15006), followed by donkey anti-rabbit FITC-conjugated secondary Ab diluted 1:400 (Jackson 711-096-152), or by donkey anti-Rabbit PE-conjugated secondary Ab diluted 1:200 (Jackson, 711-116-152), and analyzed by Flow Cytometry (FACS).

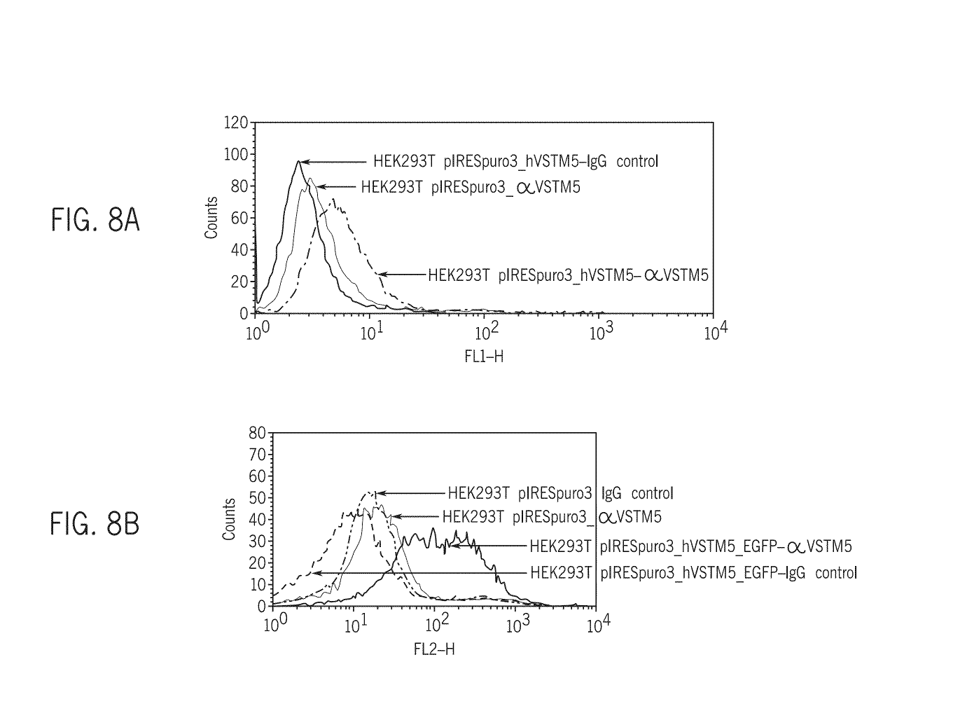

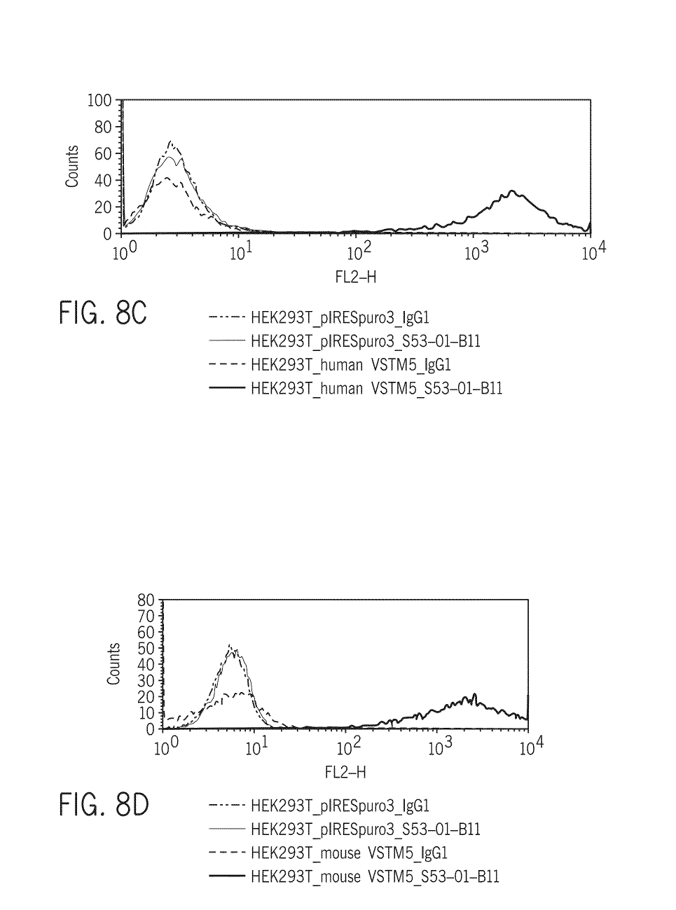

To further verify the cell-surface expression of human VSTM5 and mouse VSTM5 in HEK293T recombinant cells, as shown in FIG. 8C and FIG. 8D, respectively, cells expressing the protein were stained with monoclonal VSTM5 antibody (S53-01-B11, described in Example 12 herein) and appropriate Isotype control followed by Goat a human PE conjugated Ab (Cat#109-116-098) and analyzed by Flow Cytometry.

Results

Stable Pool of HEK-293T Cells Over Expressing the Human VSTM5 and VSTM5-EGFP Proteins (SEQ ID NOs:132 and 133, Respectively)

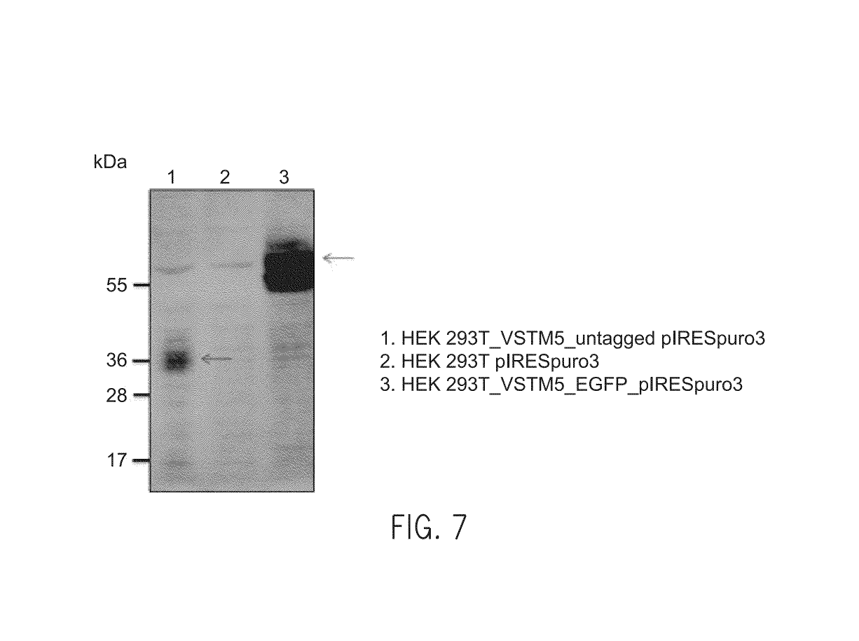

To verify expression of the VSTM5 proteins (SEQ ID NOs:132 and 133) in the stably transfected HEK293T cell pools, whole cell extracts of stable pools expressing VSTM5 or VSTM5-EGFP proteins (SEQ ID NOs:132 or 133, respectively) were analyzed by western blot using a commercial rabbit pAb, as described in Materials & Methods herein above. FIG. 7 presents the results of the western blot analysis of ectopically expressed human VSTM5 proteins using an anti-VSTM5 antibody. Whole cell extracts (30 ug) of HEK293T cell pools, previously transfected with expression construct encoding human VSTM5 (lane 1), empty vector (lane 2) or with expression construct encoding human VSTM5-EGFP (lane 3), were analyzed by WB using an anti-VSTM5 antibody. The results, shown in FIG. 7, demonstrate a specific band corresponding to the expected protein size of ˜30 kDa or ˜60 kDa in the extracts of HEK293T cell pools expressing human VSTM5 or VSTM5-EGFP (SEQ ID NOs:132 or 133, respectively), but not in the cells transfected with the empty vector.

In order to verify cell surface expression of the VSTM5 proteins, HEK293 stably transfected cells over-expressing the human VSTM5 or VSTM5-EGFP proteins (SEQ ID NOs:132 or 133, respectively) were analyzed by flow cytometry (FACS) using anti-VSTM5 pAbs. FIG. 8 presents the results of cell surface expression of human VSTM5 (A) and VSTM5-EGFP (B) proteins by FACS analysis. The anti-VSTM5 pAb (10 ug/ml) was used to analyze HEK-293T cells stably expressing the human VSTM5 proteins. Rabbit IgG was used as Isotype control to the pAb. Cells expressing the empty vector (pRp=pIRESpuro3) were used as negative control. Detection was carried out by donkey anti-rabbit FITC or PE-conjugated secondary Ab and analyzed by FACS.

Particularly, as shown in FIG. 8A and FIG. 8B, binding of the rabbit anti-VSTM5 pAb to cells stably expressing the human VSTM5 or human VSTM5-EGFP proteins was considerably higher than that observed with cells transfected with the empty vector, or cells stained with the rabbit IgG control, indicating specific cell membrane expression of the VSTM5 proteins.

Confirming Expression of Human VSTM5 Untagged in HEK293T Recombinant Cells by FACS

To verify the cell-surface expression of human VSTM5 in HEK293T recombinant cells (FIG. 8C), cells expressing the protein were stained with monoclonal VSTM5 antibody (S53-01-B11, described in Example 12 and 13 herein) and appropriate Isotype control followed by Goat a human PE conjugated Ab (Cat#

FIG. 8C demonstrates membrane expression of human VSTM5 protein by using 1 nM (0.15 ug/ml) monoclonal VSTM5 Ab (S53-01-B11) compared to 1 nM (0.15 ug/ml) IgG1 control antibody followed by PE-Goat a human secondary conjugated Ab in 1:200 dilution and analyzed by Flow Cytometry. Non expressing cell line (HEK293T_pIRESpuro3) was stained under the same conditions and used for a negative control.

Confirming Expression of Mouse VSTM5 Untagged in HEK293T Recombinant Cells by FACS

To verify the cell-surface expression of mouse VSTM5 in HEK293T recombinant cells (FIG. 8D), cells expressing the protein were stained with monoclonal anti VSTM5 antibody (S53-01-B11, described in Example 12 herein) and appropriate Isotype control followed by Goat a human PE conjugated Ab (Cat#109-116-098) and analyzed by Flow Cytometry.

FIG. 8D presents membrane expression of mouse VSTM5 protein by using 1 nM monoclonal (S53-01-B11) VSTM5 antibody compared to 1 nM IgG1 control antibody followed by PE-Goat a human secondary conjugated Ab in 1:200 dilution and analyzed by Flow Cytometry. Non expressing cell line was stained under the same conditions and used for a negative control.

### Example 3

### In-Vitro Testing of the Effect of VSTM5, Expressed on HEK 293T Cells, on the Activation of Jurkat Cells

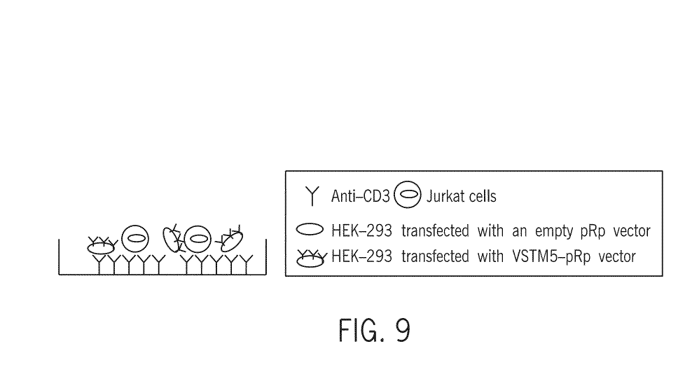

In order and as shown in FIG. 9 in order to evaluate the effect of the native cell surface expressed VSTM5 protein on T cell activation, we used a co-culture assay using HEK-293T cells over expressing VSTM5 (the generation of which is described in Example 2) and Jurkat cells (derived from a human T cell leukemia) activated by plate-bound anti-CD3 antibodies.

Materials and Methods

Effect of Cell Surface Expressed VSTM5 on Anti-CD3 Mediated Activation of Jurkat T Cells as Measured by CD69 Expression.

HEK-293T cell pools stably expressing VSTM5 or transfected with the empty vector, as described in Example 2 herein, were treated with mitomycin C (Sigma, M4287, 0.5 mg/ml, freshly prepared in H₂O) at a final concentration of 50 μg/ml, for 1 hour at 37° C. Mitomycin C treated HEK-293T cells were washed, harvested by addition of 1 ml of cell dissociation buffer (Gibco; Cat. 13151-014), resuspended in Jurkat cells' growth medium, and diluted to 0.5×10⁶ cells per ml. Cells were serially diluted and seeded at the indicated concentrations in 100 μl per well, in a flat-bottom 96-well plate pre-coated with anti-hCD3 (Clone UCHT1, BD Bioscience; cat#555329, at 2 μg/ml, overnight at 4° C.). After 2 hours to allow HEK-293T cells attachment, 50,000 Jurkat cells (ATCC, clone E6-1, TIB-152) were added to each well at a volume of 100.11 per well. Cells were co-cultured O.N. at 37° C. in a humidified incubator.

For assessment of CD69 upregulation (early marker of T cell activation) on Jurkat cells, cells were transferred to U-shape plates, and flow cytometry (FACS) analysis of cell surface expression levels of CD69 was carried out using an anti-CD69 Ab (Biolegend, PE-anti human CD69, clone FN50, cat#310906, 10 μg/ml, 2 μl/well) and Fc-blocker (Miltenyi Biotec, human FcR blocking reagent, cat#120000-442, Readouts were Mean Fluorescence Intensity (MFI) or percentage of cells expressing CD69 out of total T cells. Jurkat cells were gated according to Forward Scatter (FSC) vs. Side Scatter (SSC). Gating procedure was validated by staining the cells with anti-CD2 antibody (Biolegend; clone RPA-2.10, Cat. 300206) to identify the Jurkat T cells.

Results

Inhibition of Anti-CD3-Mediated Activation of Jurkat Cells as Measured by CD69 Expression.

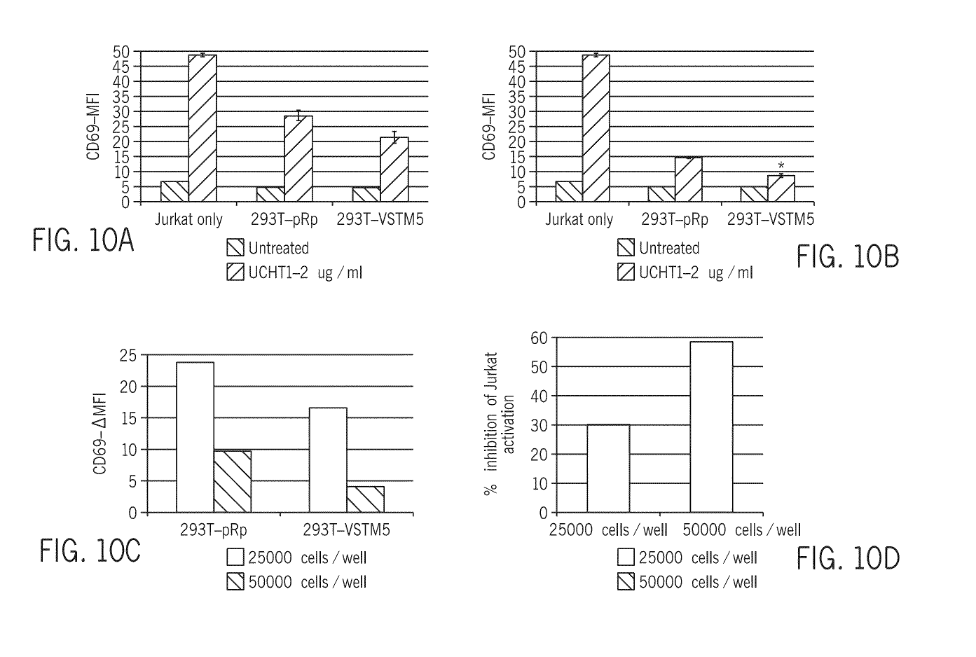

HEK-293T transfectants expressing the VSTM5 protein fused to EGFP (SEQ ID NO:133) were co-cultured with Jurkat cells activated by plate-bound anti-CD3 antibodies, as described in Materials and Methods herein. HEK-293T cells transfected with the vector only (pRp3) were used as a negative control. Representative results, shown in FIG. 10, indicate that Jurkat T cells stimulated with anti-human CD3 antibodies exhibit reduced activation in the presence of VSTM5-expressing HEK-293T cells, as manifested by lower levels of CD69 (an early marker of T cell activation) in comparison to the effect of HEK-293T cells transfected with the vector only (pRp3). The inhibitory effect of VSTM5 was best detected when using 50,000 (as compared to 25,000) HEK-293T transfected cells per well.

These results show that VSTM5 expressed on the cell membrane of HEK-293T cells inhibits Jurkat T cell activation, and indicate that the native VSTM5 membrane protein expressed on the cell surface inhibits T cell activation. This inhibition of T cell activation corroborates the therapeutic potential of VSTM5 targeting agents. For example, it suggests that antibodies which agonize VSTM5 may be used to treat T cell-driven autoimmune diseases, such as rheumatoid arthritis, multiple sclerosis, psoriasis and inflammatory bowel disease, as well as other immune related conditions wherein there are pathogenic T cells or wherein reducing undesirable immune activation is desired such as after gene therapy or transplant. In addition, these results also support the therapeutic potential of immunopotentiating VSTM5 targeting agents that reduce the inhibitory activity of VSTM5 (e.g. VSTM5 directed antibodies) for treating conditions which would benefit from enhanced immune responses such as cancer, infectious diseases, particularly chronic infections and sepsis.

### Example 4

### In-Vitro Immunomodulatory Activities of VSTM5 ECD-Ig on Mouse T Cells

In these experiments we investigated the immunomodulatory activities of the recombinant fused protein VSTM5-ECD-Ig on mouse T cells activation. In order to evaluate the activity of VSTM5 ECD-Ig protein on T cell activation assays, a recombinant protein comprising the extracellular domain of human (H) or mouse (M) VSTM5 fused to the Fc of mouse (M) IgG2a (designated VSTM5-ECD-Ig H:M (SEQ ID NO: 131, described in PCT/US2008/075122) or VSTM5-ECD-Ig M:M (SEQ ID NO:8). The effect of VSTM5-ECD-Ig on activation of mouse CD4 T cells was investigated using a number of in-vitro T cell activation readouts: cell activation markers, cytokine secretion and proliferation.

Materials & Methods

VSTM5-ECD Fusion Proteins and Control Ig

VSTM5-ECD-Ig H:M (SEQ ID NO: 131) or VSTM5-ECD-Ig M:M (SEQ ID NO: 8) were used in these assays. Mouse IgG2a Isotype control (clone C1.18.4; BioXCell) was used as control Ig.

Mouse CD4⁺ T Cell Isolation

CD4⁺CD25⁻ T cells (1 step negative selection) or naïve CD4⁺CD25⁻CD62L⁺ (1 step negative selection, followed by positive selection) T cells were isolated from pools of spleens and lymph nodes of BALB/C by using a T cell isolation Kit (Miltenyi Cat#130-093-227) according to the manufacturer's instructions. The purity obtained was >90%.

Activation of Mouse CD4⁺ T Cells

Anti-mouse CD3-ε mAb (clone 145-2C11; BD Pharmigen) at 2 ug/ml was co-immobilized overnight at 4° C. alone or together with VSTM5-ECD-Ig or control Ig, at various concentrations, on 96-well flat bottom tissue culture plates (Sigma, Cat. # Z707910). Wells were washed 3 times with PBS and plated with 1-2.5×10⁵ purified CD4⁺CD25⁻ or naïve CD4⁺CD25⁻CD62L⁺ T cells per well and kept in a humidified, 5% CO₂, 37° C. incubator. Culture supernatants were collected at the indicated times post stimulation and analyzed for mouse IFNγ or IL-2 secretion by ELISA kits (R&D Systems). The effect of VSTM5-ECD-Ig H:M (SEQ ID NO: 131) on mouse CD4⁺ T cells proliferation was determined by labeling CD4⁺CD25⁻ T cells with CFSE (0.5 μM; Molecular Probes Cat. #C34554) and analyzing cell division's profiles at 72 h post stimulation. The effect of VSTM5-ECD-Ig M:M (SEQ ID NO: 8) on the expression of the activation marker CD69 on mouse CD4⁺ T cells was analyzed by flow cytometry. Cells were stained 48 h post stimulation with a cocktail of antibodies including PerCP-anti-CD4 (clone G41.5; Biolegend), FITC-anti-CD69 (clone H1.2F3; Biolegend) in the presence of anti-CD16/32 (clone 2.4 g2; BD Biosciences) for blocking of Fcγ-receptors. Cells were evaluated using BD FACS Calibur and data analyzed using BD CellQuest Software.

Results

VSTM5-ECD-Ig Inhibits Mouse T Cell Activation

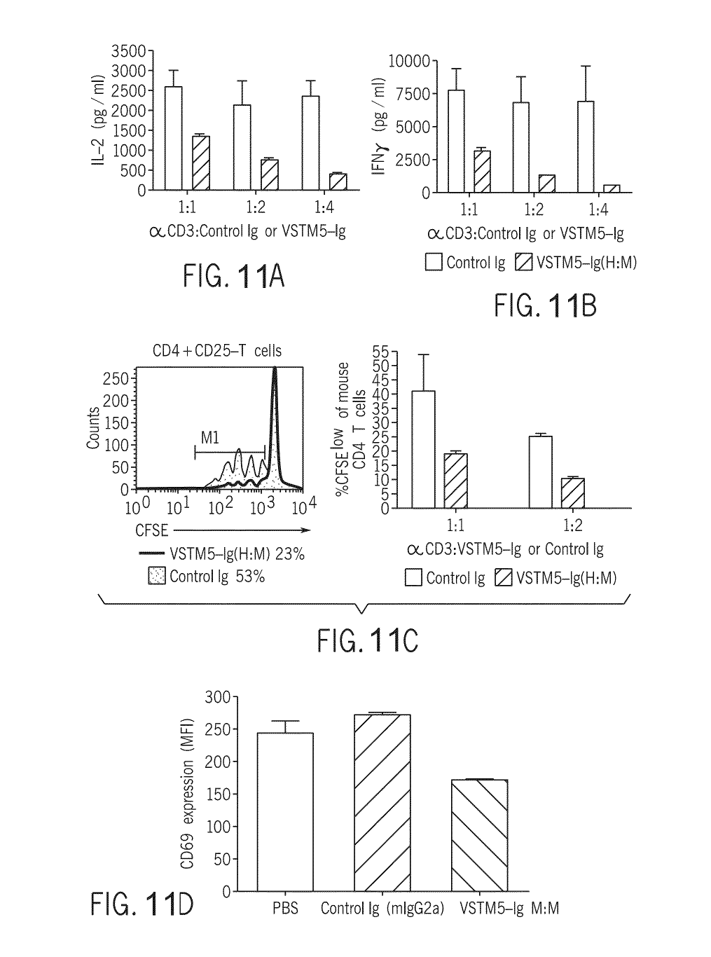

VSTM5-ECD-Ig H:M (SEQ ID NO: 131) and VSTM5-ECD-Ig M:M (SEQ ID NO: 8) were used to evaluate the immunomodulatory role of VSTM5 on mouse T cell activation upon co-immobilization on microplates with anti-CD3; mouse IgG2a was used as negative control. Results are shown in FIG. 11. VSTM5-ECD-Ig H:M (SEQ ID NO: 131) suppressed mouse CD4 T cell activation as manifested by reduction in TCR-induced cytokine secretion (IL-2 and IFNγ; FIGS. 11A and B, respectively), and in cell division (CFSE dilution, FIG. 11C). VSTM5-ECD-Ig M:M (SEQ ID NO: 8) was also shown to suppress upregulation of activation marker CD69 in mouse CD4⁺ T cells upon TCR stimulation (FIG. 11D).

The above described functional assays used to evaluate the activity of VSTM5-ECD-Ig on mouse T cells, demonstrate the inhibitory effect of VSTM5-ECD-Ig on mouse T cells activation, manifested by reduced cytokine secretion and cell proliferation, and suppression of activation marker CD69 upregulation. This inhibition of T cell activation, similarly to that observed with the native membrane bound VSTM5 protein, in Example 3, supports the therapeutic potential of VSTM5 targeting agents according to the present invention (e.g. VSTM5 directed antibodies) in treating T cell-driven autoimmune diseases, such as rheumatoid arthritis, multiple sclerosis, psoriasis and inflammatory bowel disease, as well as for other immune related diseases and/or for reducing the undesirable immune activation that follows gene therapy. In addition, these results also support the therapeutic potential of VSTM5 targeting agents that reduce the inhibitory activity of VSTM5 for treating conditions which should benefit from enhanced immune responses, in particular enhanced CTL immunity and proinflammatory cytokines such as cancer, infectious diseases, particularly chronic infections and sepsis wherein T cell-mediated depletion of diseased cells is therapeutically advantageous.

### Example 5

### Effect of VSTM5 ECD on Human T Cells Activated Using Anti-Cd3 and Anti-CD28 in the Presence of Autologous PBMCs

In these experiments the effects of VSTM5 on human T cells activated using anti-CD3 and anti-CD28 in the presence of autologous PBMCS was evaluated.

Materials & Methods

Proteins and Reagents

VSTM5 ECD fused to human IgG1 Fc (VSTM5 ECD-Ig H:H, SEQ ID NO: 130) was produced by ExcellGene in CHO-DG44 cells. CD4⁺ Human T cell Isolation Kit II was purchased from Miltenyi (Cat. #130-094-131). hIGg1 control (Synagis) was obtained from Medimmune Inc. Anti-human CD3 Ab (OKT3, Cat#16-0037) and anti-human CD28 Ab (clone CD28.2; Cat#16-0289) were purchased from eBioscience. Dynabeads® M-450 Epoxy (Cat.#140.11) were purchased from Invitrogen. Buffy coats of human blood were obtained from LifeSource. Ficoll-Paque Plus (Cat. #17-1440-02), was purchased from GE HealthCare.

Human T Cell Activation

Human PBMCs of three healthy human donors' blood were isolated from buffy coats using Ficoll separation. Total PBMCs were suspended in Ex-Vivo 20® medium, and irradiated at 3000 rad. Naïve CD4⁺ T cells were isolated from buffy coats using CD4⁺ Human T cell Isolation Kit II (Miltenyi) according to the manufacturer's instructions, and co-cultured with irradiated autologous PBMCs at a ratio of 1:1 (1.5×10⁵ T cells with 1.5×10⁵ irradiated PBMCs per well). The cultures were activated with anti-CD3 (0.5 ug/ml) and anti-CD28 (0.5 ug/ml) antibodies. VSTM5 ECD-Ig (SEQ ID NO: 130) or hIgG1 control Ig (Synagis) were added to the culture at the indicated concentrations. After 24 hr in culture, cells were pulsed with H3-thymidine. Cells were harvested after 72 hours in culture.

Results

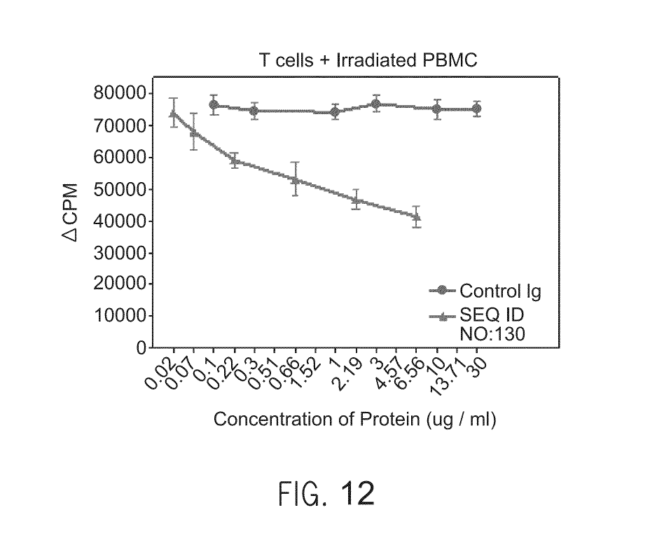

As shown in FIG. 12, addition of VSTM5 ECD-Ig (SEQ ID NO: 130) to cultures of naïve T cells from two different human donors activated by anti-CD3/anti-CD28 in the presence of irradiated autologous PBMCs resulted in a dose dependent inhibition of T cell proliferation. This inhibition of T cell activation is similar to that observed with the native membrane bound VSTM5 protein, in Example 3, and to that observed with the VSTM5 ECD-Ig on mouse T cells in Example 4.

Review

The above results support the therapeutic potential of VSTM5 targeting agents (e.g. VSTM5 directed antibodies) for treating T cell-driven autoimmune diseases, e.g., by way of example rheumatoid arthritis, multiple sclerosis, psoriasis and inflammatory bowel disease, as well as for treating other immune related diseases and/or for reducing the undesirable immune activation that follows gene therapy. Essentially, antibodies that agonize VSTM5 should prevent or reduce the activation of T cells and the production of proinflammatory cytokines involved in the disease pathology of such conditions.

In addition, these results also support a therapeutic potential of VSTM5 targeting agents that reduce the inhibitory activity of VSTM5 (e.g. VSTM5 directed antibodies) for treating conditions which will benefit from enhancing T cell mediated immune responses, such as immunotherapy of cancer, infectious diseases, particularly chronic infections and sepsis. Essentially, antibodies that antagonize VSTM5 should promote the activation of T cells and elicit the production of proinflammatory cytokines thereby promoting the depletion of cancerous or infected cells or infectious agents.

### Example 6

### Assays to Investigate the Binding Capacity of VSTM5-ECD-Ig to Human H9 T Cell Line

The aim of this study was to evaluate the binding of VSTM5-ECD-Ig to H9 cells, a human T cell line, and to use such binding capacity as a basis for development of a screening assay to identify the neutralizing potential of VSTM5 antibody fragments, e.g., Fabs or intact anti-VSTM5 antibodies, e.g., human or humanized antibodies, by measuring reduction in binding of VSTM5-ECD-Ig to the human T cell line H9.

Materials & Methods

Cell Line:

H9 cells were purchased from ATCC (ATCC cat no: HTB-176) and cultured in complete media (CM): RPMI (Gibco #21870-092) supplemented with 10% FBS (Gibco #16000-044), Glutamine (Gibco #25030-081) and Penstrep (Gibco #15070-063).

Fc-Fusion Proteins and Isotype Controls:

Human VSTM5 ECD fused to human IgG1 Fc (VSTM5-ECD-Ig H:H) (SEQ ID NO: 130) was made at GenScript; mouse VSTM5 ECD fused to mouse IgG2a Fc (VSTM5-ECD-Ig M:M) (SEQ ID NO: 8) was made at ExcellGene; human IgG1 isotype control (#ET901) was purchased from Eureka Therapeutics, USA; Synagis (Palivizumab, anti-RSV humanized IgG1 mAb) was purchased from Caligor Rx; biotinylated human IgG-Fc isotype control (#009-060-008) was purchased from Jackson Immunoresearch; mouse IgG2a isotype control, clone MOPC-173 (unlabeled #400224 and biotinylated #400204), was purchased from Biolegend, USA.

Reagents:

BiotinSP-AffiniPure Goat anti-human IgG, Fcγ specific (#109-065-098), biotinSP-AffiniPure® goat anti-mouse IgG, Fcγ2a specific (#115-065-206) and streptavidin-AF647 (SA-AF647 #016-600-084) were purchased from Jackson Immunoresearch; Dulbecco's PBS (DPBS, Life Technologies #14190-250); BSA (Sigma Aldrich #A12153); Biotinylation of hVSTM5-ECD-Ig was performed using the EZ-Link Sulfo-NHS-LC-Biotin reagent (Pierce #21327) according to manufacturer's instructions.

Binding Assay:

The binding of VSTM5-ECD-Ig (H:H or M:M, SEQ ID NOs: 130 or 8, respectively) to H9 cells was detected by FACS using either a two-step or three-step detection protocol. H9 cells were harvested when at a confluency between 0.6 to 1. 2×10⁶ cells/ml. Cells were plated at 50,000 cells per well in a 96 well v bottom plate, pelleted and the supernatant flicked off. Binding with VSTM5-ECD-Ig and its detection was carried out as described below.

Two-Step Detection

Biotinylated VSTM5-ECD-Ig H:H or biotinylated human IgG control were titrated in FACS buffer (0.5% BSA/DPBS) by performing an 8 point 3-fold serial dilution ranging from 550 nM to 0.3 nM, and 50 ul added to wells for 45 minutes at 4° C. After one wash in FACS buffer, 50 ul of 1:150 dilution of streptavidin-AF647 made up in FACS buffer was added and incubated in the dark for 20 to 30 minutes at 4° C. Following two washes in FACS buffer, samples were read on a BD Bioscience FACS Calibur with a Cytek HTS or an Accuri Intellicyte HTFC.

Three-Step Detection

Unlabeled VSTM5-ECD-Ig H:H, VSTM5-ECD-Ig M:M, human IgG1 isotype control or mouse IgG2a isotype control were titrated in FACS buffer by performing an 8 point 3-fold serial dilution ranging from 550 nM to 0.3 nM, and 50 ul added to wells for 45 minutes at 4° C. Following one wash in FACS buffer, 50 ul of biotin-anti human IgG Fcγ specific or biotin-anti mouse IgG Fcγ2a specific antibody was added for 30 minutes at 4° C. After one wash in FACS buffer, 50 ul of 1:150 dilution of streptavidin-AF647 made up in FACS buffer was added and incubated in the dark for 20 to 30 minutes at 4° C. Following two washes in FACS buffer, samples were read on a BD Bioscience FACS Calibur with a Cytek HTS or an Accuri Intellicyte HTFC.

Competition Assay

Prior to labeling H9 cells with biotinylated VSTM5-ECD-Ig, cells were incubated with increasing concentrations of unlabeled VSTM5-ECD-Ig H:H or human IgG isotype control (ET901, Eureka Therapeutics), in 50 ul for 45 minutes at 4° C. Following centrifugation, supernatant was removed and 50 ul of biotinylated VSTM5-ECD-Ig H:H at a fixed concentration of 44 nM, was added for 45 minutes at 4° C. After one wash in FACS buffer, 50 ul of 1:150 dilution of streptavidin-AF647 made up in FACS buffer was added and incubated in the dark for 20 to 30 minutes at 4° C. Following two washes in FACS buffer, samples were read on a BD Bioscience FACS Calibur with a Cytek HTS or an Accuri Intellicyte HTFC.

FACS Analysis

Data was analyzed in FCS Express (DeNovo), exported to Excel and plotted in GraphPad Prism. Data shown here is representative of two to five experiments.

Results

Human and Mouse VSTM5-ECD-Ig Bind to H9 Cells

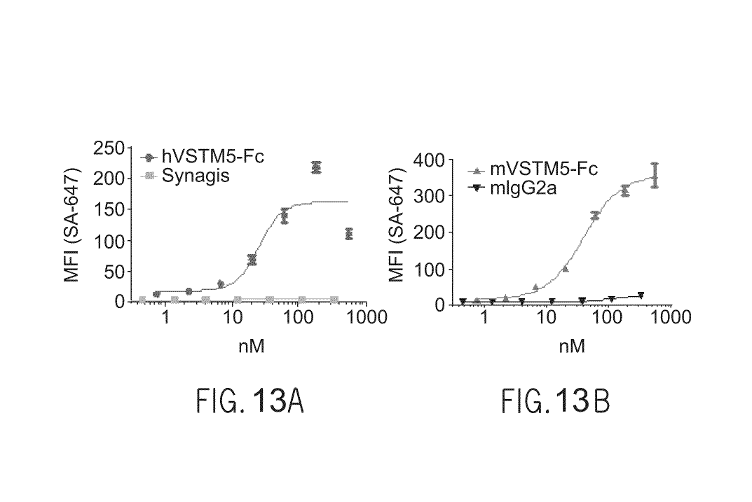

VSTM5-ECD-Ig binding to H9 cells was evaluated using the human or mouse ECD fused to a human or mouse Fc (H:H or M:M, SEQ ID NOs: 130 or 8), respectively. As shown in FIG. 13, using a 3 step labeling method, both human (FIG. 13A) and mouse (FIG. 13B) VSTM5-ECD-Ig bind H9 cells in a dose-dependent manner, while their respective isotype controls show no binding.

Specific Binding of VSTM5-ECD-Ig to H9, as Shown by its Ability to Compete Off by Itself

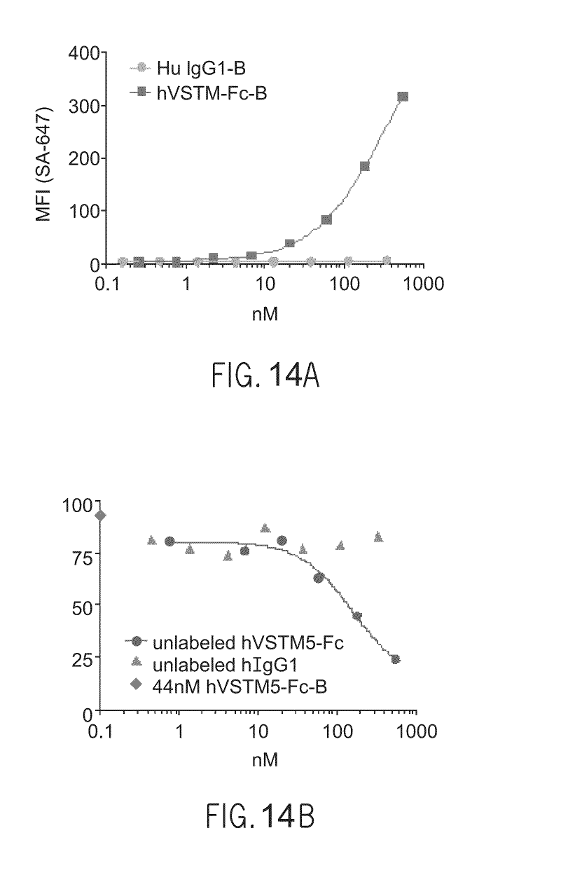

Biotinylated VSTM5-ECD-Ig H:H binds H9 cells in a dose dependent manner, using a two-step detection method (FIG. 14A). A concentration of 44 nM of biotinylated VSTM5-ECD-Ig H:H (FIGS. 14A and 14B) was used in order to evaluate specific binding to H9 cells. Unlabeled VSTM-ECD-Ig H:H indeed reduced binding of the biotinylated VSTM5-ECD-Ig with increasing concentrations, whilst the isotype control does not (FIG. 14B).

Review

VSTM5-ECD-Ig (H:H or M:M, SEQ ID NOs: 130 or 8) binds to human H9 T cell line in a dose-dependent and specific manner, suggesting the presence of a counterpart receptor in these cells. Based thereon this specific binding assay may be used to screen for the VSTM5 monoclonal antibodies that potentially may be used to neutralize or potentiate the inhibitory effect of VSTM5 on T cell activation.

### Example 7

### Binding of VSTM5-ECD-Ig Fusion Protein to Resting and Activated Human T Cells

In these experiments a soluble recombinant fusion protein, comprised of the extracellular domain (ECD) of human VSTM5 fused to the Fc domain of human IgG1, was tested in unlabeled form for binding to primary resting and stimulated human CD4⁺or CD8⁺ T cells. The Materials & Methods used in these experiments are described below.

Materials & Methods

Fc Fusion Proteins and Control Ig

The VSTM5-ECD-Ig fusion protein according to the invention, i.e., comprised of the extracellular domain (ECD) of human VSTM5 fused to the Fc domain of human IgG1 was tested for binding to resting and activated human T cells. A human B7H1-Fc protein (also known as PDL1-Fc), composed of the ECD of human B7-H1 fused to the Fc of human IgG1 (Cat#156-B7-100; R&D systems) was used as positive control for binding to activated T cells. Synagis (also known as palivizumab, Medimmune) was used as human IgG1 isotype control.

Human T Cells

Human primary CD4⁺ or CD8⁺ T cells were enriched from buffy coats of a healthy donors using RosetteSep™ Human T Cell Enrichment Cocktail (Stem Cell Technologies), according to manufacturer's instructions. The purity obtained was >95%. Following enrichment, cells were cryo-preserved (90% FCS, 10% DMSO) until future use after one freezing-thawing cycle.

Activation of T Cells and Binding Assay

Frozen human CD4⁺ or CD8⁺ T cells (Donor Q and donor D, respectively) were thawed, washed, and re-suspended at 0.5×10⁶ cells/ml in lymphocyte complete medium (CM) consisting of Iscove's Modified Dulbecco Medium (cat#01-058-1A, Biological Industries) supplemented with 10% (v/v) inactivated fetal calf serum (FBS, Biological Industries), 2 mM 1-glutamine, 100 U/ml penicillin, 100 μg/ml streptomycin, 1 mM sodium pyruvate, and 1% non-essential amino acid. 96-well plates (Costar Cat#3599) were pre-coated with PBS in the absence or presence of 1 ug/ml anti-CD3 (clone UCHT-1; R&D systems) for 4 h, at 37° C. 200 μl of T cells suspension at 0.5×10⁶ cells/ml, as described above, was added per well. Plates were placed in a humidified, 5% CO₂, 37° C. incubator for 72 hours.

Cells were harvested and stained with viability dye (Fixable Viability Stain 450, cat#562247, BD Biosciences), washed with PBS and incubated for 1 hr at room temperature (RT) with B7H1-Ig (R&D systems; cat#156-B7-100), VSTM5-ECD-Ig or control Ig (hIgG1—Synagis, Medimmune) at 100 ug/ml (50 μl/well) in FACS buffer (0.5% BSA, 2 mM EDTA, 0.05% NaN₃ in PBS). Cells were washed 3 times and stained with PE-conjugated anti-hIgG (Cat#109-116-098, Jackson laboratory; diluted 1:100) in final volume of 50 μl, 30 min 4° C. Data acquisition was performed with MACSQuant® Analyzer 10 (Miltenyi) and data analyzed using FlowJo software (version 10).

Results

Binding of VSTM5 Fusion Protein to Stimulated Human CD4⁺ and CD8⁺ T Cells

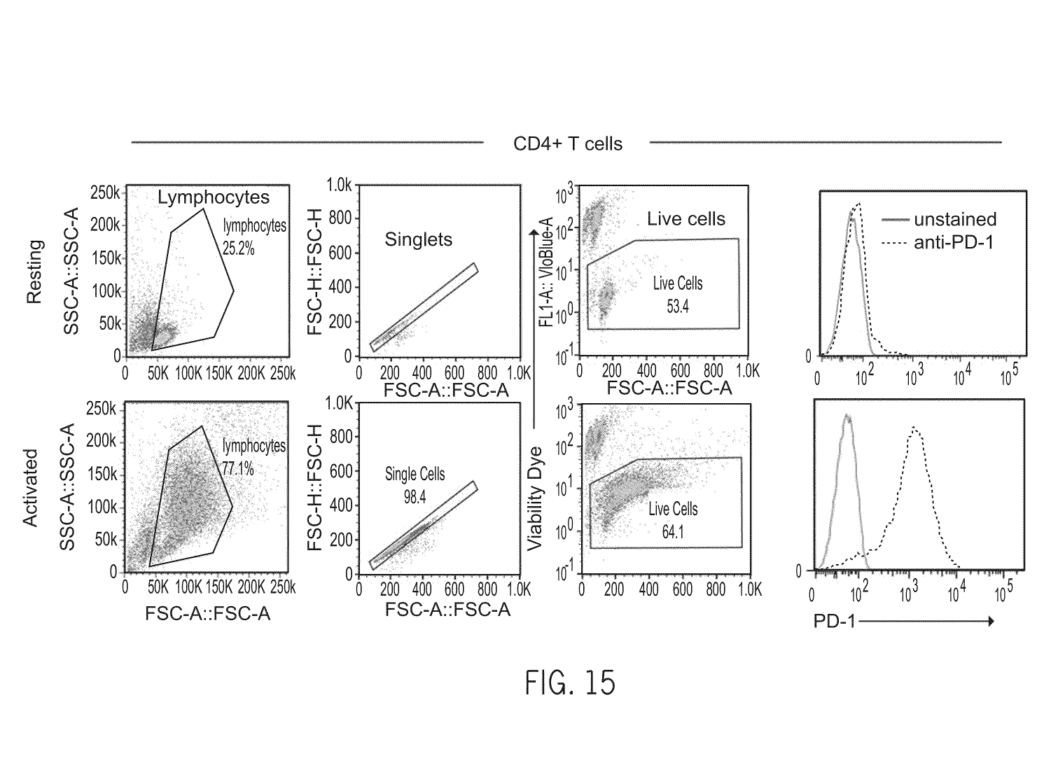

Isolated human CD4⁺ or CD8⁺ T cells were left untreated (resting) or stimulated with immobilized anti-CD3 (1 ug/ml) for 3 days as described in Materials and Methods supra. The gating strategy for flow cytometry analysis of resting and activated CD4⁺ T cells is shown in FIG. 15. Cells were first gated for lymphocytes (FSC-A vs. SSC-A), followed by singlets gate (FSC-H vs. FSC-A), and further gated for live cells. PD-1 surface expression was then determined from this gated population. As shown therein, PD-1 expression was seen on activated CD4⁺ T cells, but not on resting CD4⁺ cells, verifying the activated status of these cells. A similar gating strategy was used for resting and activated CD8⁺ T cells (data not shown).

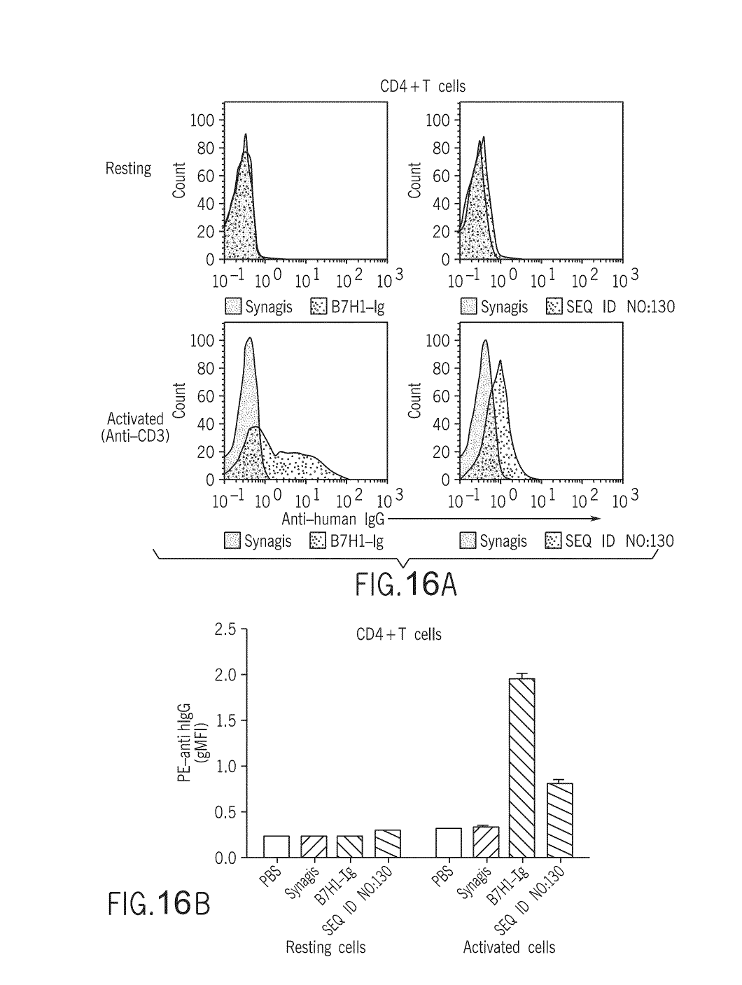

The results presented in FIGS. 16(A) and (B) show binding of unlabeled VSTM5-ECD-Ig fusion protein to anti-CD3 activated, but not resting, human CD4⁺ T cells. In these experiments isolated human CD4⁺ T cells were left untreated or stimulated with immobilized anti-CD3 (1 ug/ml) for 3 d as described in Materials & Methods supra. Cells were incubated with B7H1-Ig, VSTM5-ECD-Ig or control Ig (Synagis) at 100 ug/ml, and evaluated by flow cytometry, as described in Materials & Methods. The results in FIG. 16 (A) show the binding of B7H1-Ig and VSTM5-ECD-Ig, compared to control Ig, to resting and activated CD4⁺ cells, following gating for singlets and live cells as described in FIG. 15. In these experiments B7-H1-Ig was used as a positive control since it is a known ligand of PD-1, which is up-regulated upon T cell activation. FIG. 16(B) contains the values of histograms obtained in this experiment representing the geometric mean fluorescent intensity (gMFI) of resting and activated CD4⁺ cells. Each bar is the mean±SD of duplicate samples. One representative experiment out of two independent experiments performed is shown in the Figure. The data in FIG. 16(B) shows that there was with a 2.5 fold increase in the geometric MFI (gMFI) compared to Ig control (gMFI values, VSTM5-ECD-Ig: 0.81 vs. control Ig: 0.335) on activated CD4+ cells, while there was no clear difference in resting cells. Also B7H1-Ig bound to activated, but not to resting CD4⁺ T cells as expected, with a 6 fold increase in the gMFI compared to Ig control (FIG. 16(B); gMFI values, B7H1-Ig 1.9 vs. 0.335 of Ig control).

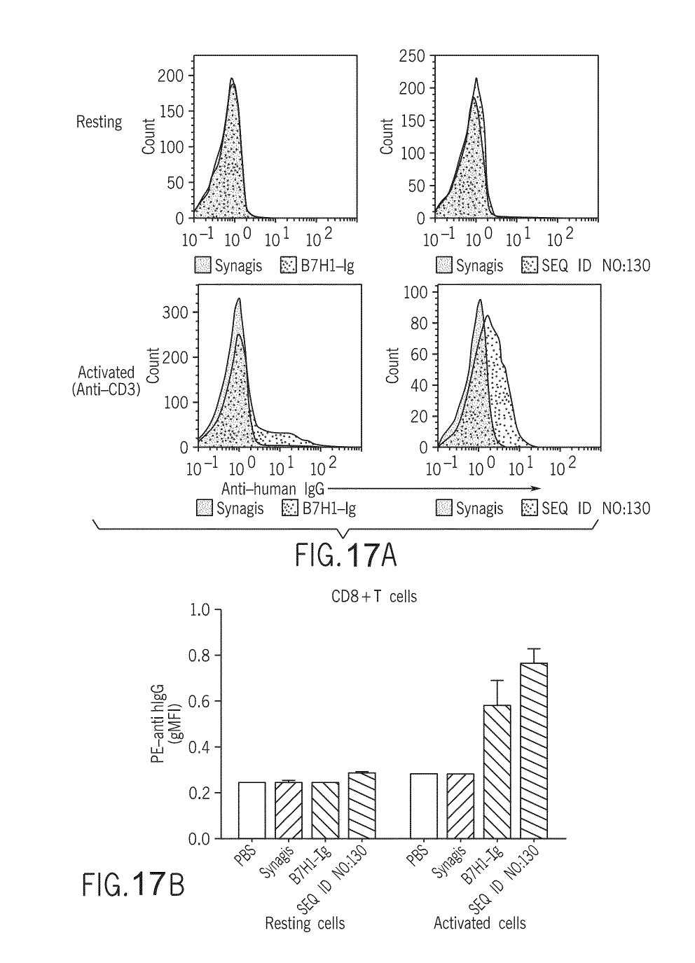

The binding of VSTM5-ECD-Ig to resting and activated human CD8⁺ cells was also evaluated. In the experiments shown in FIG. 17, isolated human CD8⁺ T cells were left untreated or stimulated with immobilized anti-CD3 (1 ug/ml) for 3 d as described in the Materials & Methods supra. Cells were incubated with unlabeled B7H1-Ig or VSTM5-ECD-Ig fusion protein, compared to control Ig (Synagis), at 100 ug/ml, and evaluated by flow cytometry, as described in Materials and Methods. FIG. 17(A) shows the binding of B7H1-Ig and VSTM5-ECD-Ig and a control Ig to resting and activated CD8⁺ cells following gating for singlet and live cells, as described in FIG. 15. FIG. 17(B) contains the values of histograms represent the geometric mean fluorescent intensity (gMFI) of resting and activated CD8⁺ cells. Each bar is the mean±SD of triplicate samples. One representative experiment out of two independent experiments performed is shown. As shown in FIGS. 17 (A & B) a similar binding pattern to CD4⁺ T cells was observed: i.e., VSTM5-ECD-Ig bound to activated, but not to resting CD8⁺ T cells. In these experiments the binding of VSTM5-ECD-Ig to activated CD8⁺ cells was comparable to that of B7-H1-Ig (FIG. 17; gMFI values, B7H1-Ig: 0.76, VSTM5-ECD-Ig: 0.58, control Ig: 0.28).

Review

The results of these experiments further demonstrate the binding of a soluble VSTM5-ECD-Ig fusion protein to activated human CD4⁺ and CD8⁺ T cells, which were pre-stimulated in a TCR-dependent manner No binding was detected when resting CD4⁺ or CD8⁺ T cells were examined Compared with B7-H1-Ig, VSTM5-ECD-Ig demonstrated lower binding to activated CD4⁺ T cells and similar binding to activated CD8⁺ T cells. These data further corroborate the likely expression of an inducible counterpart receptor for VSTM5 on activated T cells. These findings are similar to other B7/CD28 family negative receptors, such as CTLA4 and PD-1, which also are upregulated on T cells following activation.

### Example 8

### In-Vitro Immunomodulatory Activities of VSTM5 ECD-Ig on Mouse Inducible Tregs (iTregs)

The aim of this example was to investigate the effect of the mouse VSTM5-ECD-Ig, i.e. VSTM5 ECD fused to the Fc of mIgG2a, M:M (SEQ ID NO:8), on the induction of mouse iTregs following CD4 T cell activation under iTreg driving conditions.

Materials & Methods

Isolation of Mouse CD4⁺ T Cells

CD4⁺CD25⁻ T cells were negatively isolated from C57BL/6J mouse spleen cell suspensions with CD4⁺ T-cell enrichment isolation kits (Stem Cell Technologies), and further purified by flow cytometry to >99% purity. CD4⁺CD25⁻ T cells were labeled with CFSE (0.5 μM; Molecular Probes Cat. #C34554) according to manufacturer's instructions.

Isolation of Mouse CD11c⁺ Dendritic Cells

CD11c⁺ dendritic cells (DCs) were isolated from spleen cell suspensions to >90% purity by magnetic separation using mouse CD11c positive selection kits (StemCell® Technologies).

Activation of Mouse CD4⁺ T Cells Under iTreg Driving Conditions in the Presence of Antigen Presenting Cells

Anti-mouse CD3-ε mAb (clone 145-2C11; BD Pharmingen) was immobilized overnight at 4° C., at 5 μg/ml, on 96-well flat-bottom tissue culture plates (Sigma, Cat. # Z707910). Wells were washed 3 times with PBS, and plated with 2×10⁴ purified CD11c⁺ dendritic cells at a final volume of 100 μl. Murine IgG2a Fc fused mouse VSTM5 ECD (SEQ ID NO:8) was added to a final concentration of 10 μg/ml and kept in a humidified, 5% CO₂, 37° C. incubator for 1 hour. 1×10⁵ CD4⁺ T cells were added to a final volume of 200 μl to give a 1:5 APCs to T cells ratio. Soluble anti-CD28 was added at 1 μg/ml. Then, IL-2 (5 ng/ml) and TGF-β (3 ng/ml) were added. Cells were maintained in plastic tissue plates at 37° C. in a humidified atmosphere with 5% CO₂, and analyzed by flow cytometry four days later.

Activation of CD4⁺ T Cells in the Presence of iTreg Driving Conditions (without Antigen Presenting Cells)

96-well flat bottom tissue culture plates (Sigma, Cat. # Z707910) were coated with anti-CD3 mAb (2 ug/mL) and VSTM5-ECD-Ig (SEQ ID NO:8) or control Ig control (MOPC-173, Biolegend) at 10 ug/ml. CD4⁺CD25⁻ T cells were thawed and added to wells (0.5×10⁵/well) in the presence of soluble anti-CD28 (1 ug/ml), TGF-β (Cat#7666-MB; R&D systems) and IL-2 (Cat#202-IL; R&D systems) at the indicated concentrations. On Day 5 post stimulation the percentage of CD4⁺CD25⁺FoxP3⁺ cells was assessed by flow cytometry.

Results

VSTM5-ECD-Ig M:M (SEQ ID NO:8) Enhances Induction of iTregs

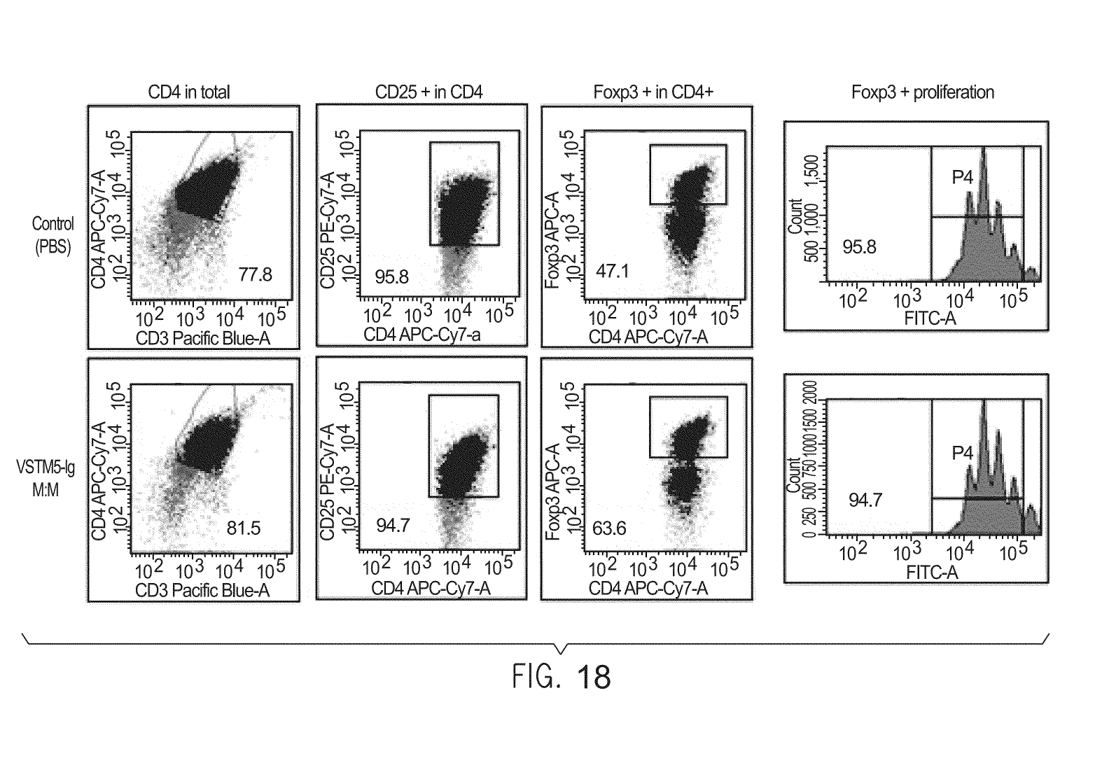

The effect of VSTM5-ECD-Ig M:M (SEQ ID NO: 8) on the induction of mouse iTregs was evaluated following T cell activation under iTreg driving conditions in the presence of antigen presenting cells. The results shown in FIG. 18 indicate that the addition of VSTM5-ECD-Ig M:M (SEQ ID NO: 8), upon activation of CD4 T cells with plate-bound anti-CD3 in the presence of mIL-2 and TGFβ, enhanced the induction of Foxp3⁺ iTregs by ˜50% (from 47.1% of total CD4⁺ T cells in the presence of PBS control, to 63.6% in the presence of VSTM5-ECD-Ig M:M (SEQ ID NO: 8)).

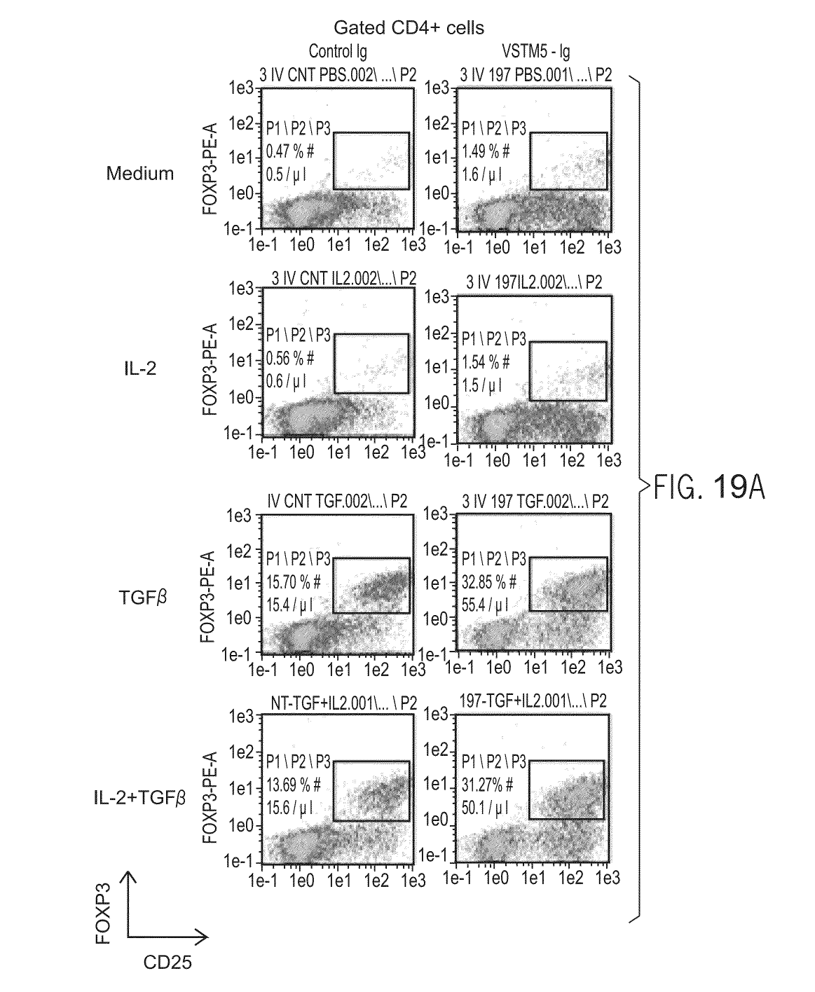

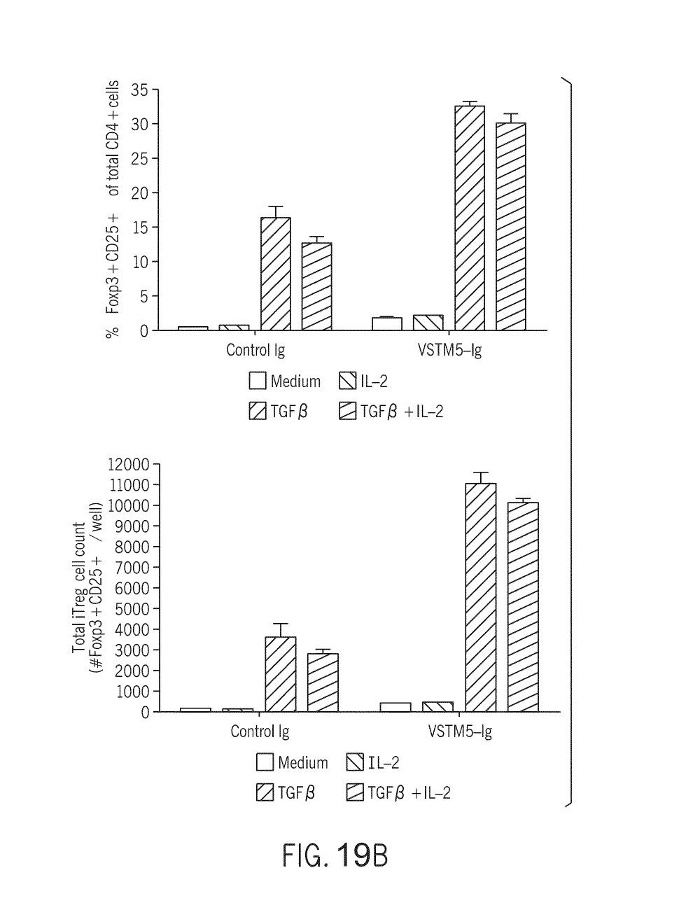

The effect of VSTM5-ECD-Ig M:M (SEQ ID NO: 8) on the induction of mouse iTregs was also evaluated following T cell activation under iTreg driving conditions, in the absence of antigen presenting cells, in an experimental set-up in which VSTM5-ECD-Ig M:M (SEQ ID NO: 8) is co-immobilized to the plate together with anti-CD3. The effect on iTreg induction was evaluated by two different parameters: the total count of CD25⁺ and FOXP3⁺ cells (presented as cell count per microliter), and the percentage CD25⁺ and FOXP3⁺ cells out of the total CD4⁺ cells. FIG. 19A presents representative plots of gated CD4⁺ cells. Values within the dot plots indicate the percentage of CD25⁺Foxp3⁺ of total CD4⁺ cells or total Tregs cell count per μl. Results summarized in FIG. 19 (B) indicate that addition of VSTM5-ECD-Ig M:M (SEQ ID NO: 8), upon activation of CD4⁺ T cells with plate-bound anti-CD3 in the presence of TGFβ with or without mIL-2, enhanced by two fold the induction of Foxp3⁺ iTregs as manifested in the percent of CD25⁺ and FOXP3⁺ cells out of total CD4⁺ cells (from 17% in the presence of Ig control, to 34% in the presence of VSTM5-ECD-Ig M:M (SEQ ID NO: 8)) and by three fold as manifested in the total count of CD25⁺ and FOXP3⁺ cells (from 3,500 Foxp3⁺ iTregs per microliter in the presence of Ig control, to 11,000 in the presence of VSTM5-ECD-Ig M:M (SEQ ID NO: 8).

### Example 9

### In Vitro Immunomodulatory Effect of VSTM5 on Human NK Cells

In these experiments we evaluated the binding potential of VSTM5-ECD-Ig HH, i.e. human ECD of VSTM5 fused to Fc of human IgG1 (SEQ ID NO:130), to NK cells; and the effect of over expression of human VSTM5 on different human cancer cell lines on their susceptibility to killing by NK cells.

Materials & Methods

Isolation of NK Cells from Peripheral Blood Mononuclear Cells

Human NK cells were isolated from PBLs (peripheral blood cells) from one healthy human donor using the human NK cell isolation kit and the autoMACS instrument (Miltenyi Biotec, Auburn, Calif.).

Generation of Primary NK Cell Clones and Polyclonal NK Cell Population

Human primary NK cell clones were obtained by seeding purified human primary NK cells at one cell/well in 96-well U-bottomed plates in complete medium supplemented with 10% FCS, 10% leukocyte-conditioned medium and 1 μg/ml PHA. Irradiated feeder cells (2.5×10⁴ allogeneic PBMCs from two donors and 5×10³ RPMI 8866 B cell line in each well) were added. Proliferating clones, as defined by growth at cell densities where growth of cells occurred in less than one third of the wells plated, were expanded in complete medium in 96-well plates. These human activated primary NK cell clones, designated as ‘NK cell clones’ herein, were cultured in RPMI, containing 10% human serum, and supplemented with 1 mM glutamine, 1 mM nonessential amino acids, 1 mM sodium pyruvate, 10000 units penicillin streptomycin and 50 U/ml rhuIL-2. The killing assays presented here were performed using a polyclonal population of NK cells (i.e. after unification of all viable NK cell clones from a certain donor).

Generation of Human Cell Lines Ectopically Expressing VSTM5

The cDNA encoding VSTM5 was cloned into the pHAGE-dsRED(−)-eGFP(+) lentiviral vector, and transduced to the following human cancer cell lines: HeLa (cervical carcinoma), RKO (colon carcinoma), RPMI-8866 (lymphoblastoid B cell line), BJAB (EBV-negative Burkitt lymphoma). The level of expression of VSTM5 in the various transduced cell lines was evaluated by FACS analysis using a commercial rabbit polyclonal antibody (Sigma, HPA029525). A rabbit IgG (SIGMA 15006) was used as isotype control, and as secondary antibody we used anti-rabbit APC-conjugated (Jackson, Cat #711-136-152).

NK Cytotoxicity Assay

The cytotoxic activity of polyclonal NK cells against various human cell lines ectopically expressing VSTM5 was evaluated using ³⁵S release assay, in which effector cells were admixed with 5×10³ [³⁵S] methionine-labeled target cells at different E:T (Effector cells to Target cells) ratios in U-bottomed microtiter plates. Following 5 hours incubation at 37° C., assays were terminated by centrifugation at 1500 rpm for 5 min at 4° C. and 50 μl of the supernatant was collected for liquid scintillation counting. Percent specific lysis was calculated as follows: % lysis=[(cpm experimental well−cpm spontaneous release)/(cpm maximal release−cpm spontaneous release)]×100. Spontaneous release was determined by incubation of the ³⁵S-labeled target cells with medium only. Maximal release was determined by solubilizing target cells in 0.1M NaOH. In all presented experiments, the spontaneous release was <25% of maximal release.

Binding to NK Cells

NK cell clones or polyclonal NK cells were incubated with 5 μg of VSTM5-ECD-Ig HH, i.e. VSTM5-ECD fused to Fc of human IgG1 (SEQ ID NO:130), or isotype control (hIgG1) for 2 hours on ice. Following cell washing, secondary anti-mouse antibody was added and binding was evaluated by flow cytometry.

Results

VSTM5-ECD Fused to Fc of Human IgG1 (SEQ ID NO:130) Binds to NK Cells

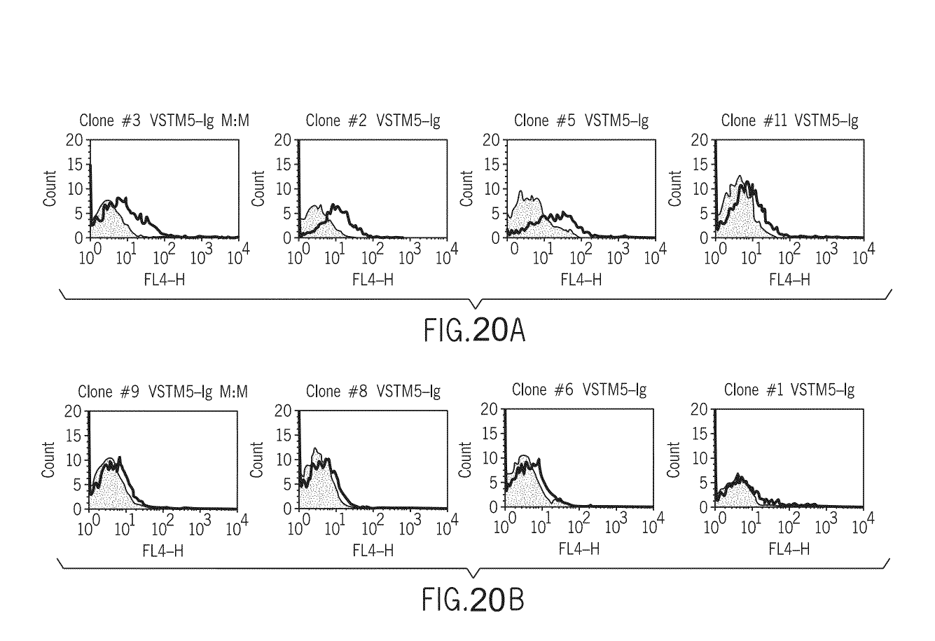

In these experiments, the binding of VSTM5-ECD-Ig, i.e. VSTM5-ECD fused to Fc of human IgG1 (SEQ ID NO:130), to activated primary NK cell clones was evaluated as described in Materials & Methods. As shown in FIG. 20, VSTM5-ECD fused to Fc of human IgG1 (SEQ ID NO:130) showed binding to activated NK cell clones at varying intensities. FIG. 20A presents clones with high binding intensities, and FIG. 20B presents clones with low binding intensities.

Over Expression of VSTM5 on Human Cells Reduces NK Cytotoxicity

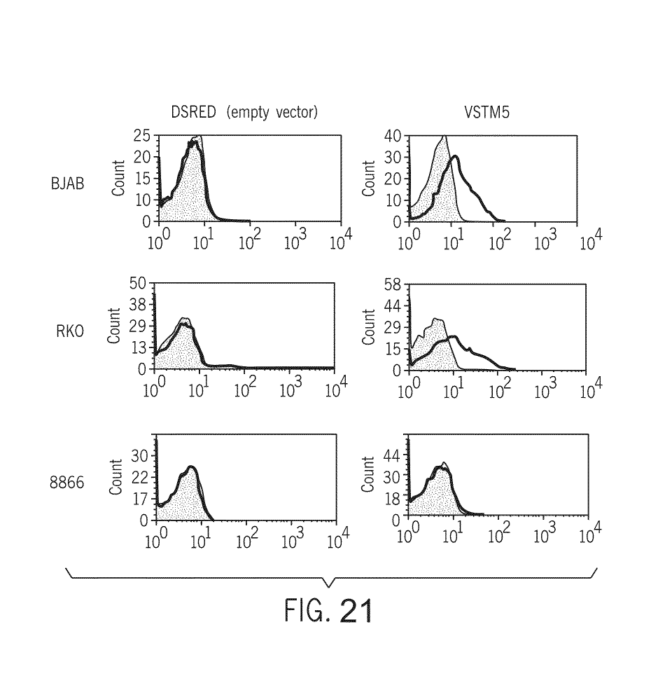

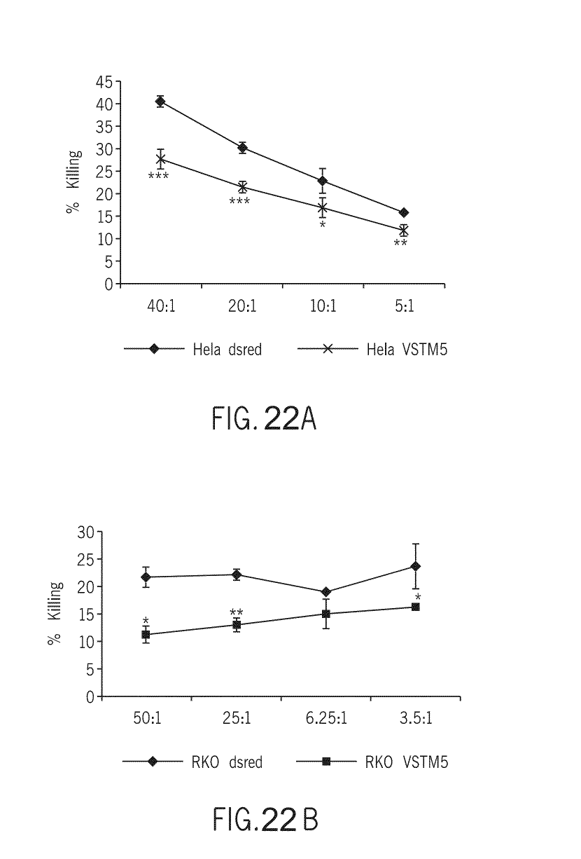

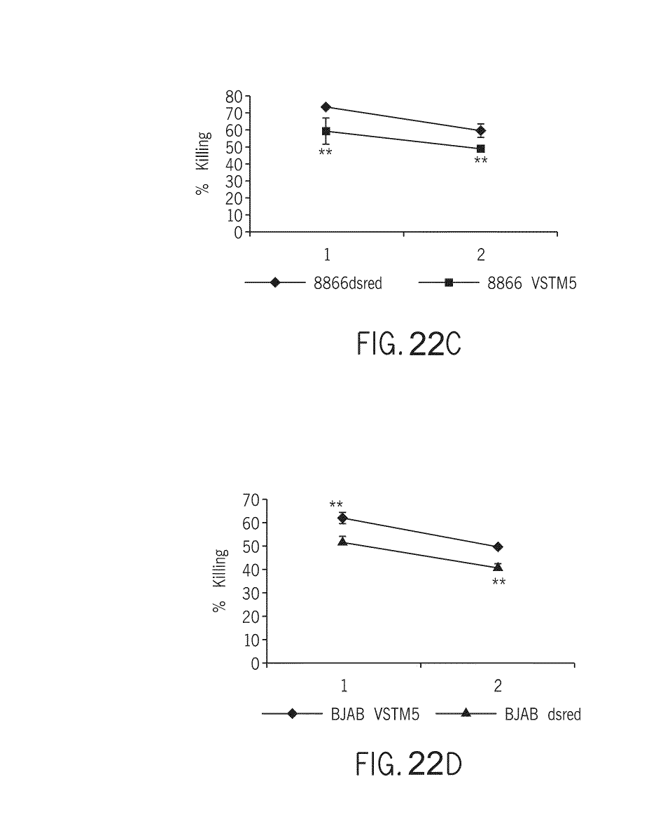

Lentiviral expression construct encoding VSTM5 (SEQ ID NO:132) was transduced to various human cancer cell lines as described in Materials and Methods. The level of expression of VSTM5 in the various cell lines was evaluated by FACS analysis and shown in FIG. 21. The effect of VSTM5 over expression on the susceptibility to killing of these target cells by NKs was assessed as described in Materials and Methods. Several experiments were carried out on these cell lines. Results of representative experiments are shown in FIG. 22, indicating that over expression of VSTM5 on these target cells (Hela—FIG. 22A, RKO—FIG. 22B, 8866—FIG. 22C and BJAB—FIG. 22D) results in a reduction of NK cells killing activity as assessed at different E:T (effector to target cells) ratios.

Review

Binding of VSTM5 fusion protein to activated NK cells was detected on several NK cell clones, indicating that a counter receptor of VSTM5 is expressed by NK cells. In co-culture experiments, over expression of VSTM5 in various human cancer cell lines reduced the killing activity of NK cells, indicating that VSTM5 has an inhibitory effect on the cytotoxic activity of NK cells.

### Example 10

### In-Vitro Immunomodulatory Activities of VSTM5 on Human Cytotoxic T Cells (CTLs)

The experiments described in this example evaluated the effect of ectopic expression of human VSTM5 (SEQ ID NO:132) on different melanoma cell lines on their ability to activate CTLs (cytotoxic T lymphocytes) and serve as targets for killing by these cells.

Materials & Methods:

General Design of the Experimental System:

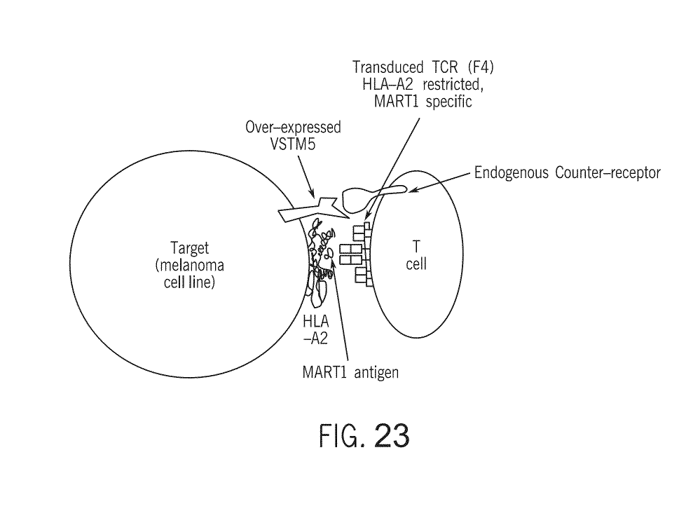

The experimental system is described in FIG. 23, VSTM5 (SEQ ID NO:132) was over expressed on human melanoma cells as target cells, which were then co-cultured with primary human CD8⁺ T cells (CTLs) over expressing a TCR (designated F4) which is specific for an antigen derived from the melanoma specific protein MART1, when presented on HLA-A2 (specific class I MHC). We subsequently evaluated the effect of VSTM5 overexpressed on melanoma cells, on MART1-specific CTLs activation F4 TCR was recently used in clinical trials in terminally-ill melanoma patients to specifically confer tumor recognition by autologous lymphocytes from peripheral blood by using a retrovirus encoding the TCR (Morgan et al, 2006 Science, 314:126-129).

Transduction and Expression of VSTM5 (SEQ ID NO:132) in Melanoma Cell Lines:

In order to express VSTM5 (SEQ ID NO:132) in target melanoma cells, the cDNA encoding VSTM5 (SEQ ID NO:132) was amplified using specific primers and cloned into an MSCV-based retroviral vector (pMSGV1). Verification of the cloning was done first using restriction enzyme digestion and subsequently by sequencing. Upon sequence confirmation, large amounts of the retroviral vector (Maxi-prep) were produced for subsequent use.

Three human melanoma cell lines which present the MART-1 antigen in HLA-A2 context (SK-MEL-23, mel-624 and mel-624.38) were transduced with the retroviral constructs encoding VSTM5 (SEQ ID NO:132) or with the empty retroviral vector. A melanoma cell line mel-888 which does not express HLA-A2 served as additional negative control. Transductions were carried out using a retronectin-based protocol; briefly, retroviral supernatant was produced in 293GP cells (a retroviral packaging cell line) following transfection with the retroviral vector and an amphotropic envelop gene (VSV-G). The retroviral supernatant was plated on retronectin-coated plates prior to the transduction to enable the binding of virions to the plate. Then, the melanoma cells were added to the plate for 6 hours. After that, the cells were replenished in a new culture vessel. Transduction efficiency and expression of the protein was determined by staining the transduced tumor cells with a commercial VSTM5-specific polyclonal antibody (Anti-VSTM5, rabbit polyclonal antibody, Sigma, Cat. No. HPA029525). A rabbit IgG (Sigma Cat. No. 15006) was used as isotype control, and as secondary antibody we used APC-conjugated anti-rabbit IgG (Jackson, 711-136-152).

Transduction of Effector Cells:

In order to obtain effector T cells that express the CD8-dependent MART-1 specific F4 TCR (a MART-126-35-specific TCR that recognizes HLA-A2⁺/MART1⁺ melanoma cells), freshly isolated human PBLs (peripheral blood leukocytes) were stimulated with PHA and cultured for 5-10 days, and subsequently transduced with a retroviral vector encoding both a and 13 chains from the F4 TCR. The transduced lymphocytes were cultured in lymphocyte medium containing 300 IU of IL-2, replenished every 2-3 days. Non-transduced T cells served as negative control.

Cytokine Secretion from F4-TCR Transduced Lymphocytes Upon Co-Culture with VSTM5 (SEQ ID NO:132)-Transduced Melanoma Cells:

Melanoma cells expressing VSTM5 (SEQ ID NO:132) or empty vector were co-cultured for 16-20 hours with F4-TCR transduced lymphocytes. Cytokine secretion (IFN-γ, IL-2 and TNFα) was measured by ELISA, to assess the specific recognition and response of the effector CD8⁺ T cells to the different transduced tumor cell lines. For these assays, 10⁵ effector cells were co-cultured with 10⁵ melanoma target cells for 16 hours. Cytokine secretion was measured in culture supernatants, diluted to be in the linear range of the ELISA assay.

Killing Assays:

The cytotoxic activity of effector cells (CTLs) against melanoma human cell lines (target cells) ectopically expressing VSTM5 was evaluated by staining for propidium iodide (PI). Effector cells were admixed with CFSE-labeled target cells at optimized E/T (Effector cells to Target cells) ratios in U-bottomed 96 well microtiter plates. Following an overnight incubation at 37° C., cells were stained with PI and read by FACS. The percentage of double positive events (stained for CFSE and PI) out of all CFSE positive events (total melanoma cells) were referred to as melanoma cells undergoing lysis. Non-transduced effector cells were used to obtain the background level of cell lysis not related to T cell specific killing activity.

Results

Over-Expression of VSTM5 on Human Melanoma Cell Lines

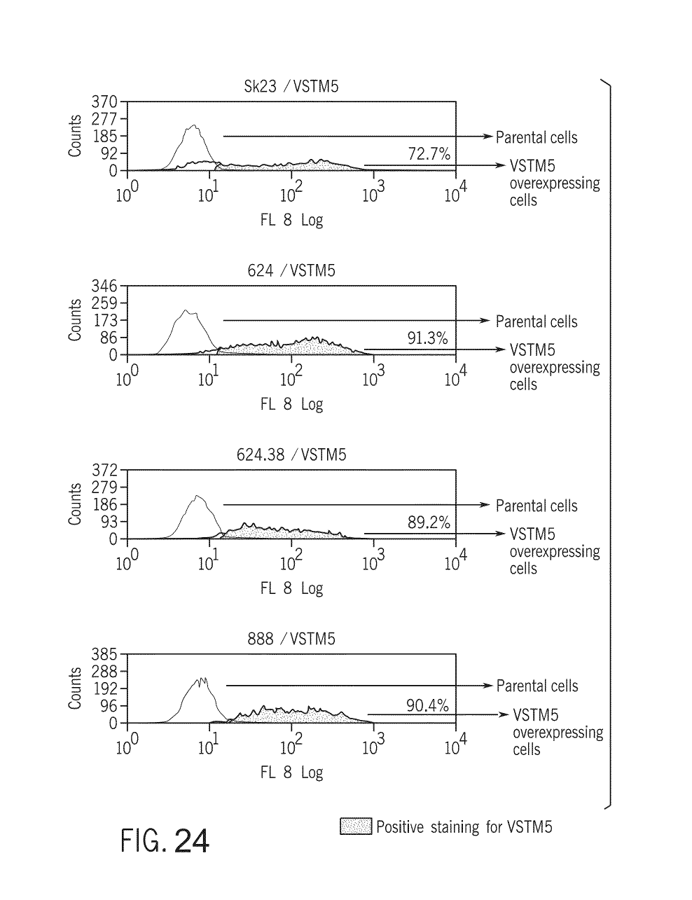

Human melanoma cell lines (SK-MEL-23, mel-624.38, mel-624 and mel-888) were stained with a VSTM5-specific monoclonal antibody. Endogenous expression of VSTM5 was not detected on the surface of these cell lines as shown by flow cytometry (data not shown). Next, these cell lines were transduced with retroviral vector encoding the VSTM5 (SEQ ID NO:132) molecule, as described in Materials & Methods herein. FIG. 24 shows the levels of VSTM5 expression as assessed by flow cytometry at 48 hrs after transduction, and compared to those of cells transduced with an empty vector. The percent of cells staining positive for the protein ranged between 70-90% for the different cell lines tested.

Over-Expression of VSTM5 on Human Melanoma Cells Reduces Activation-Dependent Cytokine Secretion from F4 Transduced CTLs

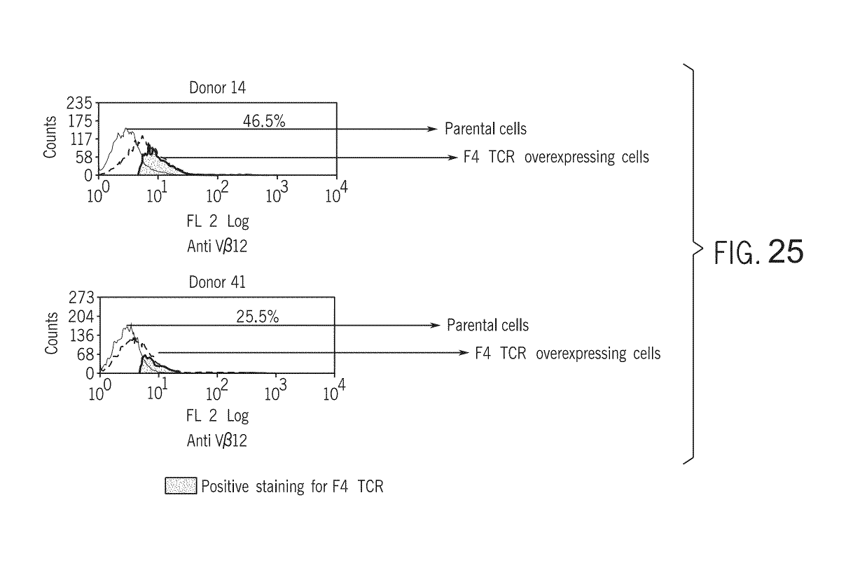

To perform functional assays with human CTLs, primary human lymphocytes were engineered to express the F4 TCR, which recognizes HLA⁻A2⁺/MART1⁺ melanoma cells, as described in the Materials & Methods. FIG. 25 shows the level of F4 TCR expression obtained upon transduction of lymphocytes from two representative donors.

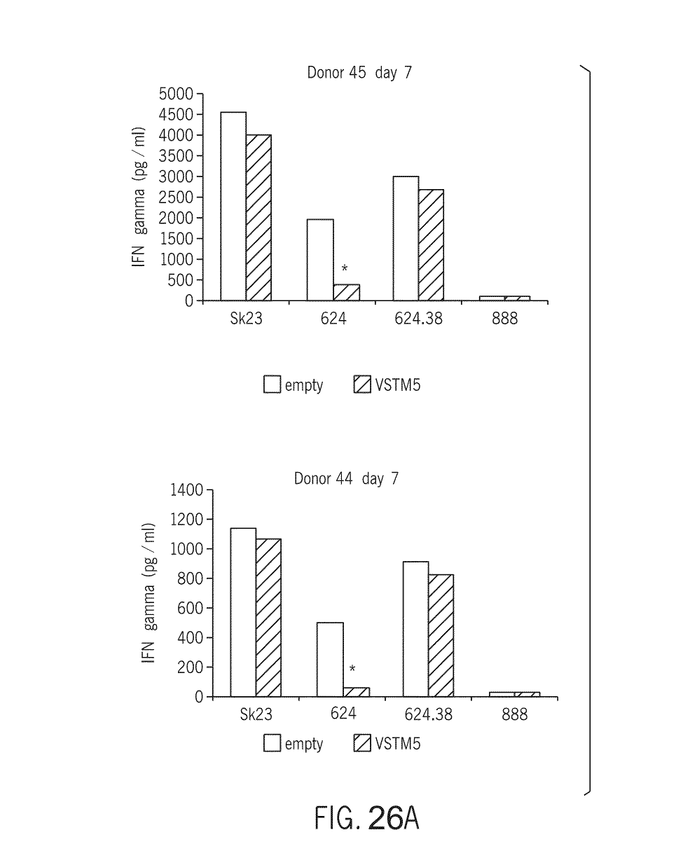

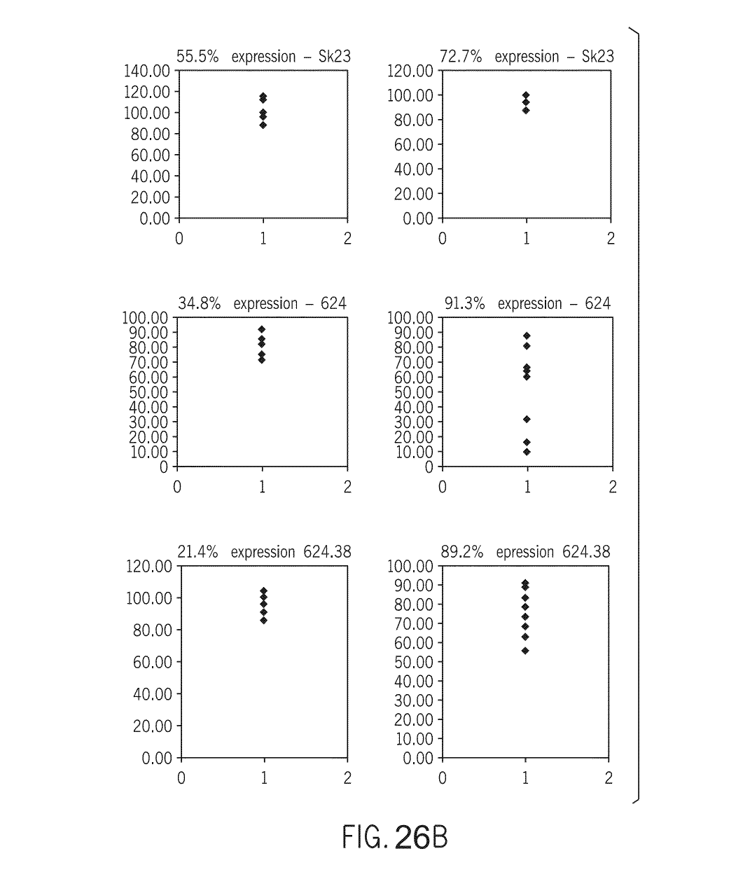

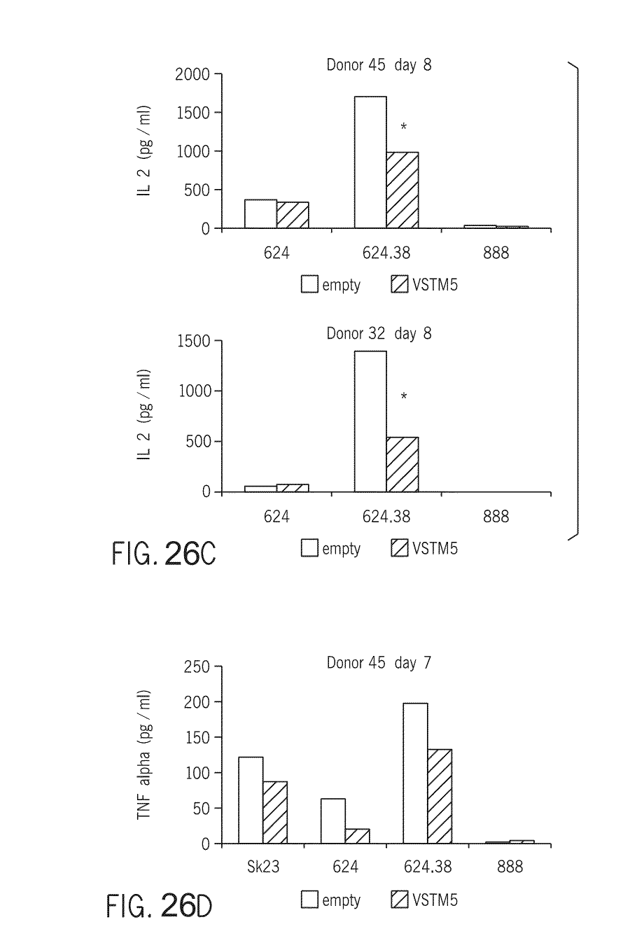

The F4 transduced effector lymphocytes, i.e. CTLs, were co-cultured with the melanoma lines expressing VSTM5 (SEQ ID NO:132) or empty vector. The levels of IFNγ, IL-2 and TNFα secretion were assessed at 16-hours of co-culture (FIGS. 26A-26D). Results shown in FIG. 26A show inhibition of IFNγ secretion from CTLs of two different donors upon co-culture with VSTM5-expressing mel-624 cells. No significant inhibition was observed when testing the effect of VSTM5-expressing SK-mel-23 or mel-624.38 cells on such CTLs. As expected, no significant secretion of IFNγ was observed in the presence of mel-888 melanoma cells (which do not express HLA-A2 and thus are not recognized by the F4 TCR), indicating an absence of activation of F4-expressing CTLs by these cells. In 8 independent experiments using 4 different T-cell donors, a significant reduction (˜30-90%) of IFNγ secretion was observed upon co-culture with VSTM5 expressing mel-624 cell line as compared to co-culture with the same cell line transduced with an empty vector (summarized in FIG. 26B). VSTM5 expressed on mel-624.38 cells, lead to a reduction in IFNγ secretion observed in several experiments but not in others. VSTM5 expressed on SK-mel-23 cells does not seem to have an effect.

The secretion of IL-2 or TNFα was also tested in a few experiments, and shown in FIG. 26C. A significant reduction (˜40-60%) was observed in IL-2 secretion from CTLs of two different donors upon co-culture with the VSTM5 expressing mel-624.38 cell line, as compared to co-culture with this cell line transfected with empty vector. In addition a reduction in TNFα secretion was observed with all three melanoma cell lines expressing VSTM5 (FIG. 26D), although only the reduction with VSTM5 expressing mel-624 reached statistical significance compared to empty vector cells. As expected, no significant secretion of IL-2 or TNFα was observed in the presence of mel-888 melanoma cells, indicating absence of activation of F4-expressing CTLs with these cells, as expected.

Over-Expression of VSTM5 on Mel-624 Human Melanoma Cells Reduces their Susceptibility to Killing by F4 Transduced CTLs.

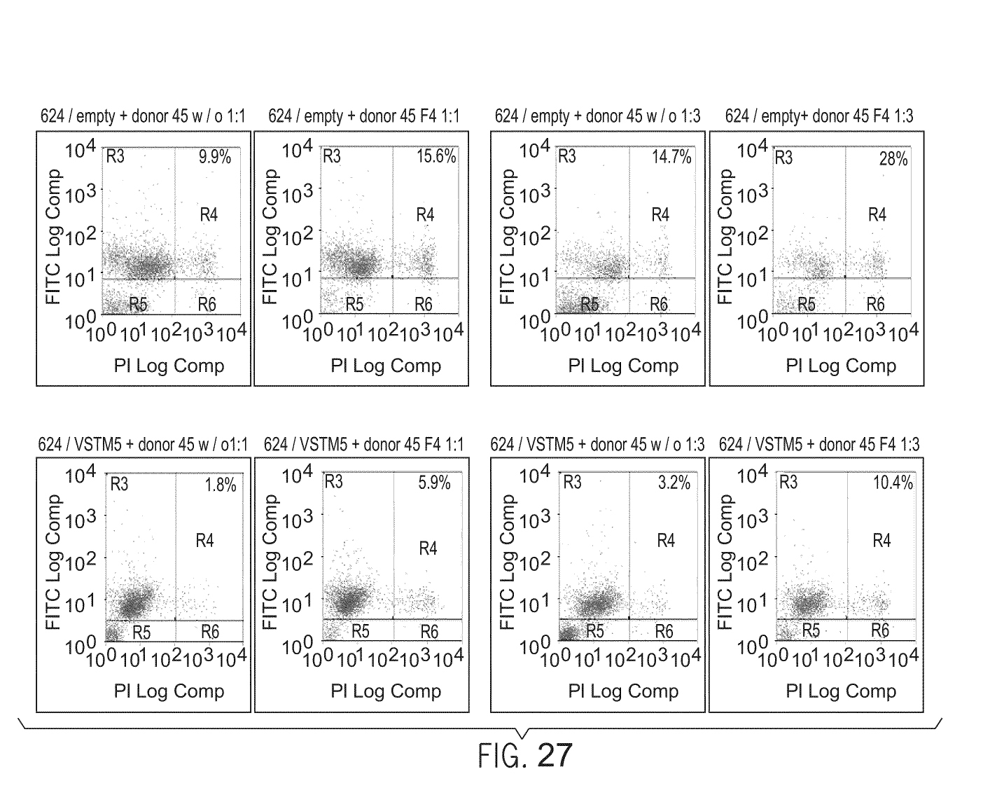

The effect of VSTM5 over expression on the susceptibility to cytotoxicity by effector CTLs was assessed by co-culture of the F4 TCR expressing lymphocytes with CFSE labeled melanoma cells as targets, following by PI staining. Percentage of double positive CFSE⁺ PI⁺ cells point to the level of target cells killing. Results are shown in FIG. 27, indicating that over expression of VSTM5 on mel-624 as target cells results in a reduction of CTL killing activity.

It should be noted that these results were not repeated in additional experiments, mainly due to technical problems having to do with set-up of the experimental system.

Review

The results of the experiments described in this example indicate that VSTM5 overexpression on melanoma cells results in reduced cytokine secretion and killing activity by CTLs, suggesting that VSTM5 has an inhibitory effect on CTLs. The difference in the effect on CTLs of VSTM5 expressed on the melanoma cell lines could be explained by a possible different repertoire of endogenously expressed co-stimulatory/co-inhibitory proteins on these cell lines.

### Example 11

### Inhibition of T Cell Activation by VSTM5-ECD-Ig Fusion Coated Bead Assay

In the experiments described in this example the inventors evaluated the effect of VSTM5-ECD-Ig fusion protein on T cell activation in a bead assay. The Materials & Methods used in these experiments are described below.

Materials & Methods

Isolation of Human T Cells

Buffy coats were obtained from Stanford Blood Bank from healthy human donors. CD3⁺ T cells were isolated from buffy coats using RosetteSep kit (StemCell Technologies) following manufacturer's instructions. Cells were >94% CD3⁺ when analyzed with anti-CD45 and anti-CD3 by flow cytometry, and >95% viable after thawing prior to the assay.

Bead Coating and QC

Tosyl activated beads (Invitrogen, Cat#14013) at 500×10⁶/ml were coated with anti-CD3 mAb and Fc fusion proteins in a two-step protocol: with 50 ug/ml human anti-CD3 clone UTCH1 (R&D systems, Cat# mab 100) in sodium phosphate buffer at 37° C. overnight, followed with 0-320 ug/ml of VSTM5-ECD-Ig fusion protein (human ECD of VSTM5 fused with Fc of human IgG1) for another overnight incubation at 37° C. In the second step, control human Fc was added together with Fc fusion protein so that the total amount of protein is 160 ug/ml (Bioxcell, Cat# BE0096) for the 0, 20, 40, 80 and 160 ug/ml coating condition (except for the 320 ug/ml coating condition).

The amount of VSTM5-ECD-Ig bound to the beads was analyzed using Alexa 647® conjugated anti-VSTM5 mab 53-01.B11 (Lot 20414), and PD-L1 Fc levels by anti-PD-L1 (ebioscience, Cat#14-9971-81) followed by goat-anti-mouse 647 (1:200) (Jackson Immuno Research, Cat#115-606-146). The amount of anti-CD3 antibody bound to the beads was analyzed using goat anti-mouse 647 (Jackson ImmunoResearch, Cat#115-606-146).

Bead Assay Setup:

100 k human CD3⁺ T cells were cultured with 100 k or 200 k beads coated with various concentrations of the VSTM5-ECD-Ig fusion protein for 5 days in complete IMDM (Gibco, Cat #12440-053) supplemented with 2% AB human serum (Gibco, Cat#34005-100), Glutmax (Gibco, Cat #35050-061), sodium pyruvate (Gibco, Cat #11360-070), MEM Non-Essential Amino Acids Solution (Gibco, Cat #11140-050), and 2-mercaptoethanol (Gibco, Cat #21985). At the end of 5 day culture, cells were stained with anti-CD25, anti-CD4, anti-CD8, and fixable live dead dye to determine CD25 expression levels on each subset of cells. Supernatants were collected and assayed for IFNγ secretion by ELISA (Human INFγ duoset, R&D systems, DY285).

Results

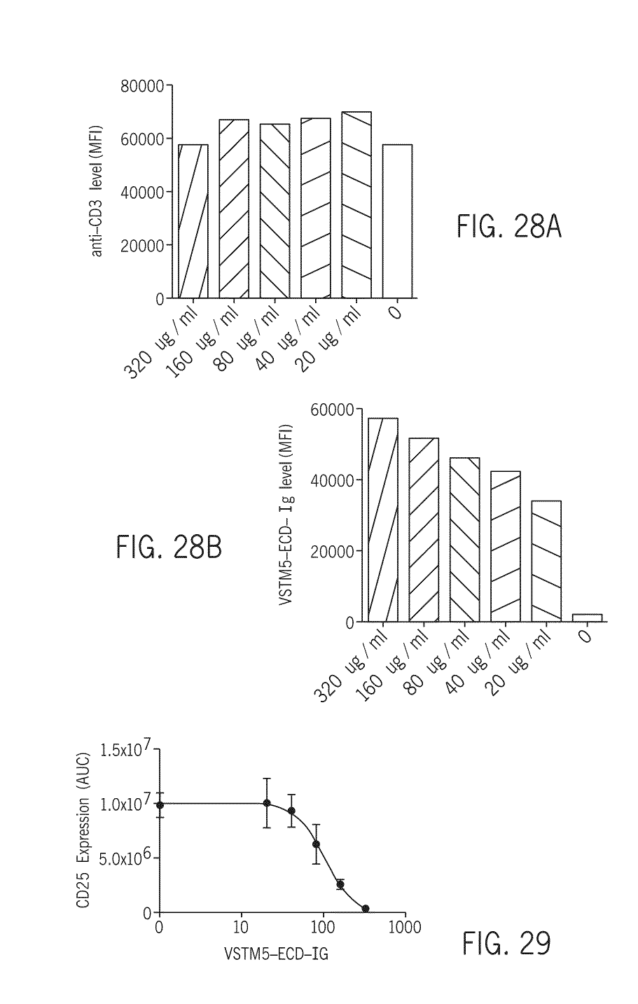

In the experiments in FIG. 28 and FIG. 29, the level of anti-CD3 and VSTM5-ECD-Ig fusion protein (SEQ ID NO:XXX) coated on the beads was determined by staining beads with anti-mouse antibody which recognize anti-CD3 and by an antibody specific for VSTM5 protein. All beads coated with various amount of VSTM5-ECD-Ig fusion protein had a similar level of anti CD3 (A), and showed increased amount of with increased concentration of in the coating solution (B).

In the experiments of FIG. 28 the beads were coated with 50 ug/ml of anti-CD3 mAb and 6 concentrations of the VSTM5-ECD-Ig fusion protein exhibited a similar level of anti-CD3 and increasing levels of VSTM5-ECD-Ig fusion protein, which levels correlated with the concentration used in the particular assay.

In the experiments in FIG. 29 human CD3 T cells co-cultured with beads coated with various concentration of VSTM5-ECD-Ig fusion protein were analyzed for their level of expression of CD25. Both CD4⁺ and CD8⁺ cells showed dose dependent inhibition by the VSTM5-ECD-Ig fusion protein, under both cell: bead ratio of 1:1 and 1:2. The data shown in FIG. 29 represents CD8⁺ T cells with cell: bead ratio of 1:1. As shown therein there was observed a dose dependent inhibition of T cell activation by VSTM5-ECD-Ig fusion under these bead conditions (1:1 ratio of cell:bead) as evidenced by the detected level of CD25 expression by the CD8⁺ T cells.

Review

The results in FIG. 28 and FIG. 29 show that VSTM5-ECD-Ig fusion proteins inhibit human T cell activation under the conditions bead assay conditions described in this example. All beads coated with various amount of the VSTM5-ECD-Ig fusion had a similar level of anti-CD3 FIG. 28(A), and showed increased amount of VSTM5-ECD-Ig fusion with increased concentration of VSTM5-ECDIg fusion in the coating solution FIG. 29(B). This observation further corroborates the potential use of VSTM5-ECD-Ig fusion proteins and other VSTM5 binding agents to modulate the immunosuppressive effects of VSTM5 on immunity, and particularly its inhibitory effect on T cell activation.

### Example 12

### Generation of High Affinity Anti-VSTM5 Fab Antibodies by Phage Display

Using the Materials & Methods described below a panel of different Fab antibodies of different epitopic specificities that bind. VSTM5 with high specificity and affinity were produced.

Materials and Methods

General Method for Direct Binding ELISA:

Unless otherwise noted, test proteins were diluted to 1 μg/mL in phosphate buffered saline (PBS) and 50 μL aliquots were coated on the wells of a Maxisorp ELISA plate (Thermo Fisher Scientific, Waltham, Mass.) overnight at 4° C., or for 1 hr at 37° C. Coated plate wells were rinsed twice with PBS and incubated with 300 μL blocking buffer (5% skim milk powder in PBS pH 7.4) at room temperature (RT) for 1 hr. Blocking buffer was removed and plates were rinsed twice more with PBS. Plate-bound proteins were detected by adding 50 μL/well of a primary antibody and incubating at RT for 1 hr. Plates were washed three times with PBS-Tween20 (PBS 7.4, 0.05% Tween20), then three times with PBS and 50 μL/well of a F(ab′)₂ fragment Specific Goat Anti-Human IgG (Jackson Immunoresearch, West Grove, Pa.) was added as the secondary detection antibody. This was incubated at RT for 1 hr and plates were washed again. Note that in some cases a HRP-conjugated primary antibody (or other detection protein) was used directly, with no secondary detection step. ELISA signals were developed in all wells by adding 50 μL of Sureblue 3,3′,5,5′-Tetramethylbenzidine (TMB) substrate (KPL Inc, Gaithersburg, Md.) and incubating for 5-20 mins. The HRP reaction was stopped by adding 50 μL 2N H₂SO₄ (VWR, Radnor, Pa.) and assay signals were read on a Fluostar (BMG Labtech, Cary, N.C.) plate reader at absorbance 450 nm.

Preparation of Biotinylated VSTM5 Antigen:

A range of proteins required for phage display experiments were biotinylated to facilitate solution-based panning. These included human VSTM5 fused to human IgG1 Fc (VSTM5HH, SEQ ID NO: 130) and mouse VSTM5 fused to mouse IgG2a Fe (VSTM5MM, SEQ ID NO: 8). A negative control human IgG1 Fc-fusion protein, i.e. irrelevant ECD fused to the same human IgG1 Fc as VSTM5HH, was biotinylated to use for depletion steps in the panning experiments (data not shown). All proteins were diluted to 1 mg/mL in 1 mL PBS, and then labeled with a Sulfo-NHS-LC-Biotin kit at a 3:1 biotin: protein ratio, as per manufacturer's instructions (Pierce, Rockford, Ill.). After conducting the binding reaction, free biotin was removed by dialyzing samples overnight against PBS pH 7.4 using 3500 MWCO Slide-A-Lyzer cassettes (Pierce). Dialyzed proteins were stored at −80° C.

Phage Panning of Human Antibody Library:

Panning reactions were carried out in solution using streptavidin-coated magnetic beads to capture the biotinylated antigens. All washing and elution steps were conducted using a magnetic rack to capture the beads (Promega, Madison, Wis.). All incubation steps were conducted at room temperature with gentle mixing on a tube rotator (BioExpress, Kaysville, Utah). As shown in Table 1 below four panning sub-campaigns were conducted, each with a different combination of antigens, washes and Fc-binder depletion steps.

TABLE 1
Antigen and Washing stringency Conditions Used
for Phage Panning against VSTM5 Antigen

| Sub- | Round | Antigen | Wash | Fc |
|---|---|---|---|---|
| A | 1 | SEQ ID NO: 130 | Long | No |
| | 2 | SEQ ID NO: 8 | | |
| | 3 | SEQ ID NO: 130 | | |
| B | 1 | SEQ ID NO: 130 | Short | No |
| | 2 | SEQ ID [symbol] | | |
| | 3 | SEQ ID NO: 130 | | |
| C | 1 | SEQ ID NO: 130 | Long | Yes |
| | 2 | | | |
| | 3 | | | |
| D | 1 | SEQ ID NO: 130 | Short | Yes |
| | 2 | | | |
| | 3 | | | |

[symbol]

indicates data missing or illegible when filed

Sub-campaigns A and B alternated between the human and mouse ECD versions of VSTM5 in an attempt to enrich binders with human/mouse species cross-reactivity. Sub-campaigns C and D focused on the human ECD version of the antigen, and used depletion steps against the negative control human IgG1 Fc-fusion protein detailed in [0012] to remove binders against the human IgG1 Fc. All campaigns used 100 pmol of the appropriate VSTM5 or negative Fc-fusion control protein per round.

Preparation of Phage Library for Panning:

All phage panning experiments used the XOMA031 human Fab antibody phage display library (XOMA Corporation, Berkeley, Calif.). Sufficient phage for a 50-fold over-representation of the library were blocked by mixing 1:1 with 10% skim milk powder in PBS (final skim milk concentration 5%) and incubating for 1 hr.

Antigen Coupling to Streptavidin Beads:

For each sub-campaign, three 100 μL aliquots of Dynal streptavidin-coated magnetic beads (Life Technologies) were blocked by suspension in 1 mL of blocking buffer (5% skim milk powder in PBS) and incubated for 30 mins. One blocked bead aliquot was mixed with an amount of biotinylated VSTM5 antigen dependent on the panning round and reaction conditions (Table 1). The other two aliquots were either mixed with 100 pmols of the negative control human IgG1 Fc-fusion protein (C and D), or not coupled to a biotinylated protein (A and B). Biotin-labeled antigens were coupled to the beads for 1 hr at RT. Bead suspensions for C and D were washed twice with PBS to remove free antigen and re-suspended in 100 μL blocking buffer. Blocked beads for A and B were washed and re-suspended in the same way.

Depletion of Human IgG1 Fc and Streptavidin Bead Binders from the Phage Library:

Unwanted binders to streptavidin beads (all sub-campaigns) and the Fc region of VSTM5HH (sub-campaigns C and D) were removed before phage panning. This was accomplished using blocked phage mixed with one 100 μL aliquot of uncoupled streptavidin beads (A and B) or beads coupled to the Fc-fusion human IgG1 control protein (C and D) and incubated for 45 mins. The beads (and presumably unwanted bead and human IgG1 Fc-binders) were discarded. This step was repeated with a second 100 μL of beads (with or without negative control protein, as appropriate) and the ‘depleted’ phage library supernatants were reserved for panning.

Phage Panning Round 1:

The blocked and depleted phage library was mixed with the VSTM5 beads described above. This suspension was incubated for 1 hr at RT with gentle rotation to allow binding of VSTM5 specific phage. Non-specific binders were removed by washing according to the protocol in Table 1. The sequences of washes in all displays was: Round 1, three washes with PBS-T and three washes with PBS; round 2 and round 3, six washes with each buffer. In the table, ‘short wash’ refers to re-suspending the beads in 1 mL of wash buffer using five aspirations with a pipette. ‘Long wash’ refers to re-suspending the beads in 1 mL of wash buffer before incubating the beads on a tube rotator for five mins.

After washing, the bound phage were eluted by incubation with 500 μL of 100 mM triethylamine (TEA) (EMD Millpore, Rockland, Mass.) for 20 mins at RT. The eluate was neutralized by adding 500 μL of 1 M Tris-HCl pH 8.0 (Teknova, Hollister, Calif.).

Determination of Phage Titer: 10 μL of the initial phage library (input titer) or panning eluate (output titer) was serially diluted (10-fold) in PBS. A 90 μL aliquot of each phage dilution was mixed with 90 μL of TG1 E. coli cells grown to an optical density of ˜0.5 at 600 nm (OD 600 nm). Phage were allowed to infect the cells by stationary incubation for 30 mins, then shaking incubation (250 rpm) for 30 mins, all at 37° C. A 10 μL aliquot of each infected cell culture was spotted on a 2YT agar plate supplemented with 2% glucose and 100 μg/mL carbenicillin (2YTCG, Teknova). Plates were incubated overnight at 30° C. Colonies growing from each 10 μL spot were counted and used to calculate input and output titers.

Phage Rescue:

The remaining phage eluate (˜1 mL) was mixed with 10 mL of TG1 E. coli grown to an OD 600 nm of 0.5. Phage were infected into cells as detailed above, infected cells were pelleted by centrifugation at 2500×G and re-suspended in 750 μL 2YT medium (Teknova). The cell suspension was divided into three equal aliquots that were spread on 2YTCG agar plates. These plates were incubated overnight at 37° C. and the resulting E. coli lawns were scraped and re-suspended in ˜20 mL liquid 2YTCG (Teknova). This cell suspension was used to make 1 mL glycerol stocks for each panning round. A small aliquot of re-suspended cells was inoculated into 50 mL 2YTCG to achieve an OD 600 nm of 0.05, and then grown at 37° C. with 250 rpm shaking until the OD reached 0.5. The resulting culture was infected with M13K07 helper phage (New England Biolabs, Ipswich, Mass.) and incubated overnight at 25° C. with shaking to allow phage packaging. The culture supernatant containing rescued phage particles was cleared by centrifugation at 2500×G and 1 mL was carried over for either a) a subsequent round of panning or b) Fab binding screens. Phage in the remaining supernatant were concentrated and purified for phage pool ELISAs (see below).

Phage Panning Rounds 2-3:

Second and third rounds of panning were conducted as per the steps above, except that the rescued phage supernatant from the previous round was used in place of the phage library. The amount of biotinylated VSTM5 used varied over the course of the experiments (Table 1).

Phage Pool Enrichment ELISA:

Phage from each panning round were precipitated from rescue culture supernatants by adding 1/5 volume of PEG-6000/NaCl solution (Teknova). Precipitated phage were harvested by centrifugation at 8000 rpm and re-suspended in 1 mL PBS. Phage aliquots were diluted 1:10 in blocking buffer (5% skim milk powder in PBS) and 50 aliquots were added to the wells of ELISA plates coated with VSTM5HH (SEQ ID NO:130), VSTM5MM (SEQ ID NO:8), or the negative control protein, human IgG1 isotype control and mouse IgG2 isotype control. Bound phage were detected with a HRP-conjugated anti-M13 phage coat antibody (GE Healthcare, Pittsburgh, Pa.) diluted 1:2000 in PBS-T. All other assays steps were conducted as described in the general ELISA protocol.

Fab expression vectors: The pXHMV-Fab-kappa and pXHMV-Fab-lambda phagemid vectors used in the XOMA031 library also function as Fab expression vectors. These vectors contain Fab heavy chain and light chain expression cassettes, a lac promoter (plac) to drive expression of the antibody genes, and an ampicillin resistance gene. The antibody chains are appended with N-terminal signal peptides to drive their secretion into the periplasmic space. The C-terminal of the heavy chain carries a truncated gene III protein sequence for incorporation into phage particles. The heavy chain also carries hexa-histidine, c-myc and V5 affinity tags. Transformation of these vectors into E. coli and induction with isopropyl 13-D-1-thiogalactopyranoside (IPTG) results in periplasmic expression of soluble Fab molecules.

Fab PPE Production:

Eluted Phage Pools from Panning Round 3 were Diluted and Infected into TG1

E. coli cells (Lucigen, Middleton, Wis.) so that single colonies were generated when spread on a 2YTCG agar plate. This resulted in each colony carrying a pXHMV-Fab vector encoding a single Fab clone. Individual clones were inoculated into 1 mL 2YTCG starter cultures in 96-well deepwell blocks (Greiner Bio-One, Frickenhausen, Germany) using a Qpix2 instrument (Molecular Devices, Sunnyvale, Calif.). These starter cultures were grown overnight in a Multitron 3 mm incubator (ATR Biotech, Laurel, Md.) at 37° C. with 1000 rpm shaking.

For Fab expression, 20 μL of 1 mL starter cultures were transferred into a second set of deepwell plates containing 1 mL 2YT with 0.1% glucose and 100 μg/mL ampicillin. Cultures were grown until the average OD 600 nm was 0.5-1.0 and protein expression was induced by adding IPTG (Teknova) to a final concentration of 1 mM. Expression cultures were incubated overnight in the Multitron instrument at 25° C. with 700 rpm shaking.

Fab proteins secreted into the E. coli periplasm were extracted for analysis. Cells were harvested by centrifugation at 2500×G, the supernatants were discarded and pellets were re-suspended in 75 μL ice-cold PPB buffer (Teknova). Extracts were incubated for 10 mins at 4° C. with 1000 rpm shaking, and then 225 μL ice-cold ddH2O was then added and incubated for a further 1 hr. The resulting periplasmic extract (PPE) was cleared by centrifugation at 2500×G and transferred to separate plates or tubes for ELISA and FACS analysis. Note that all extraction buffers contained EDTA-free Complete Protease Inhibitors® (Roche, Basel, Switzerland).

ELISA Binding Assays:

Each plate of PPE extracts was tested for binding to biotinylated VSTM5-ECD-Ig H:H (SEQ ID NO: 130) and the negative Fc-fusion control protein. The ELISA followed the general protocol above, except that the biotinylated antigen was captured on a streptavidin-coated 96-well plate (Pierce) instead of a standard ELISA plate. A 50 μL aliquot of each PPE was added to plate wells coated with these antigens and the remainder of the ELISA followed the general method. Bound Fab was detected using a HRP-conjugated anti-human Fab′₂ antibody (Jackson Immunoresearch) diluted 1:2000 in PBS with 5% skim milk.

FACS Screening of PPE:

MT vector and Human VSTM5-EGFP suspension 293 cells were cultured in 293 freestyle media (Life Technologies) supplemented with 5 μg/ml puromycin (Life Technologies). All reagent preparations and wash steps were carried out in FACS buffer (PBS (Life Technologies), 0.5% BSA (Sigma Aldrich, St. Louis, Mo.). Both cell types were combined, pelleted and resuspended at 1.5×10⁶ cells/ml of each cell line. 25 ul of cells was added to each well containing 25 μl PPE for 30 mins at 4° C. 2 ug/ml of an anti-VSTM5 Ab (Sigma #HPA029525) or rabbit IgG (Abeam, Cambridge, Mass.) were used as positive or negative controls (respectively), added at the secondary antibody step, on each plate. Plates were washed one time in 200 μl of FACS buffer. 30 μl of 1:1000 dilution of mouse anti c-myc (Roche) was added per well for 30 mins at 4° C. followed by a wash step as before.

24 μl of a 1:300 dilution of goat anti mouse Fab-AF647 (Jackson Immunoresearch, West Grove, Pa.) was added to each PPE containing well and 1:500 dilution of goat anti rabbit-AF647 (Life Technologies) added to each positive and matched negative well for 25 mins at 4° C. After two washes cells were resuspended in a final volume of 80 pal 1% paraformaldehyde made up in FACS buffer. Samples were read on a BD Bioscience FACS Calibur with a Cytek HTS, recording approximately 5000 events per well in a designated live gate. Data was analyzed using FCS Express® (De Novo Software, CA, USA) and exported to Excel® Ratio of Mean Fluorescence Intensity (MFI) of transfected cells: MFI signal of empty vector control cells was calculated and exported into Xabtracker (XOMA). Positive hits were identified as those giving an MFI ratio equal of greater than 2-fold. In addition a visual call was scored in FCS Express to corroborate the positive hits.

Re-Formatting of Fab Hits and Production as Human IgG Molecules:

Protein expression constructs for human anti-VSTM5 IgGs were derived by PCR-amplification of variable heavy, lambda and kappa domain genes, which were subcloned into pFUSE-CHIg-hG1 (human IgG1 heavy chain), pFUSE2-CLIg-hK (human kappa light chain) or pFUSE2-CLIg-hL2 (human lambda 2 light chain) vectors, respectively (all expression vectors sourced from Invivogen).

Expi293 cells (Life Technologies) were seeded at 6×10⁵ cells/ml in in Expi293™ medium (Life Technologies) and incubated for 72 hrs at 37° C. in a humidified atmosphere of 8% CO2 with shaking at 125 rpm. This cell stock was used to seed expression cultures at 2.0×10⁶ cells/nil in Expi293™ medium. These cultures were incubated as above for 24 hrs with shaking at 135 rpm.

For transfection, cells were diluted again to 2.5×10⁶ cells/ml in Expi293 medium. The protein expression constructs for antibody heavy chain and light chain were mixed at a ratio of 1:2. For every 30 mL of expression culture volume, 30 μg of DNA and 81 μL of Expifectamine (Life Technologies) were each diluted separately to 1.5 mL with Opti-MEM (Life Technologies) and incubated for five minutes. Diluted DNA and Expifectamine were then mixed and incubated at RT for 20 mins. This was then added to the expression culture in a shaker flask and incubated as described above, with shaking at 125 rpm.

Approximately 20 hrs post-transfection, 150 μL of ExpiFectamine 293 transfection Enhancer 1 and 1.5 mL of ExpiFectamine™ 293 Transfection Enhancer 2 was added to each flask. Cultures were incubated for a further five days (six clays post-transfection in total) and supernatants were harvested by centrifugation. IgGs were purified from the supernatants using an AKTA Pure FPLC (GE Healthcare Bio-Sciences) and HiTrap MabSelect Sure® affinity columns (GE Healthcare Bio-Sciences) according to the manufacturer's instructions.

FACS Screening of Reformatted IgG1 Antibodies:

Empty vector control cells and human VSTM5 EGFP suspension 293 cells were pelleted and stained in 50 μl of the indicated concentrations of anti-VSTM5 antibodies or isotype controls in FACS buffer at 4° C. for 60 mins. Cells were washed once in FACS buffer, re-suspended in 50 μl of 1:250 dilution of biotinylated anti-human IgG (Jackson cat#109-065-097) for 30 mins at 4° C. Cells were washed in FACS buffer, re-suspended in 50 μl of 1:100 dilution of SA-PE (Jackson cat#016-110-084) for 30 mins. Cells were washed twice and re-suspended in a final volume of 100 μl of FACS buffer. Samples were read on the Intellicyt HTFC. Data was analyzed by FCS Express (DeNovo, Calif., USA), exported to Excel (Microsoft, WA, USA) and plotted in GraphPad Prism® (GraphPad Software, Inc., CA, USA).

SPR Binding Assays:

All low resolution SPR assays were performed using a Biacore 3000 instrument (GE Healthcare Bio-Sciences, PA, USA) at 22° C.

Results

Fab PPE Screening:

A set of 186 Fab clones from each sub-campaign were tested by FACS for binding against HEK293 cells over-expressing the full length VSTM5 protein. A subset of 93 of these Fabs was also tested for binding against the VSTM5-ECD-Ig fusion protein used in the panning experiments. All hits were sequenced to eliminate redundant Fabs. The final set of unique Fabs was tested in duplicate confirmatory FACS assays and also tested for human and mouse VSTM5 binding by SPR. ELISA and SPR were also used to test each Fab for unwanted cross-reactivity against the human and mouse Fc regions.

The overall screening exercise yielded 315 potential binders (by FACS and/or ELISA), of which 151 were sequenced successfully. The sequence diversity (i.e. number of different sequences observed/total number sequence) was similar in all sub-campaigns, ranging from 30-38%. There were 46 different Fabs identified in total, ten of which fell into three closely related ‘sibling’ families (a sibling family contains Fabs with the same H-CDR3 sequence, but minor differences in the heavy or light chain framework regions). The best yield of unique FACS-binding Fabs came from campaigns B (7) and C (13), with a moderate yield from D (4).

IgG Reformatting and Binding Results:

A subset of 23 unique anti-VSTM5 Fabs were selected for reformatting and production as IgG molecules, based on evidence of binding against HEK293 cells expressing human VSTM5.

FACS Binding Assays:

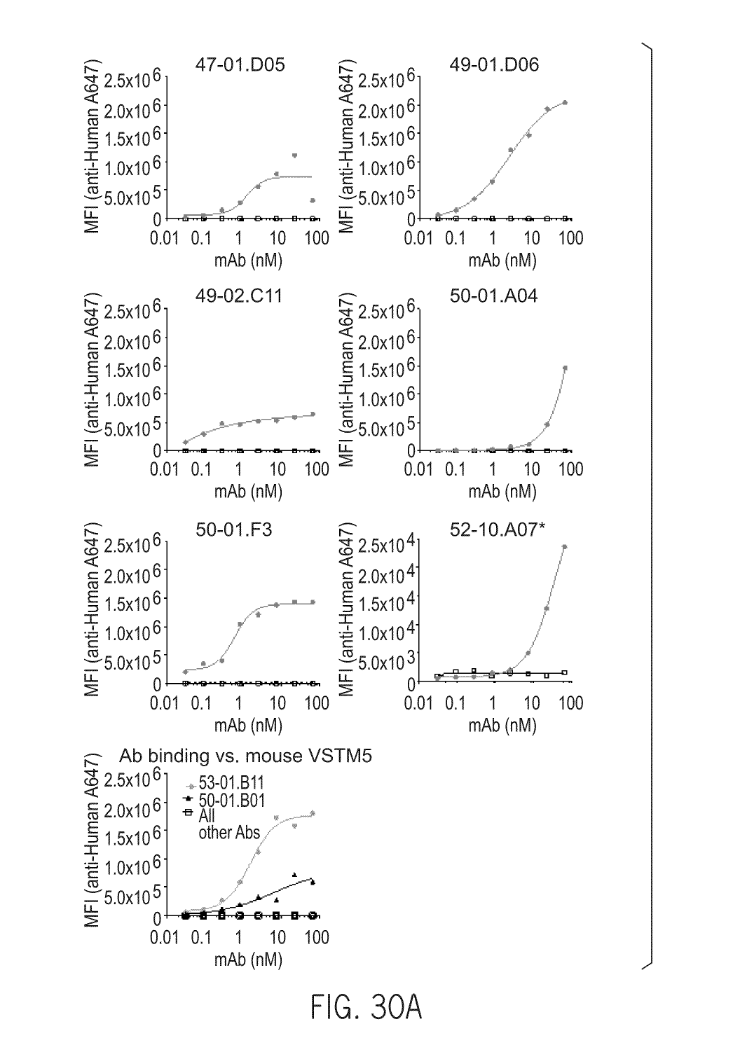

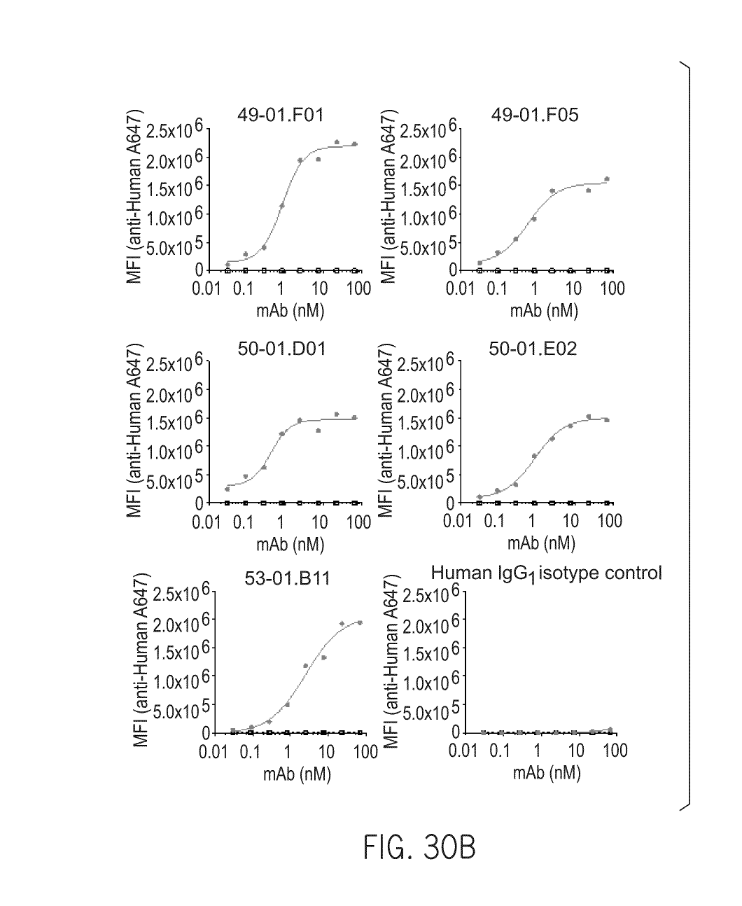

18 of these IgGs were produced and tested for binding to 293-huVSTM5 cells (as per the PPE screening experiments). Mabs were also tested for cross-reactivity against a 293-muVSTM5 cells. All mAbs were also tested for background binding against the 293-MT control cells (FIG. 30). Twelve of the tested antibodies bound specifically to the 293-huVSTM5 cell line, with two of these demonstrating cross-reactivity to 293-muVSTM5 cells (summarized in Table 2).

FIG. 30 presents FACS binding results for anti-VSTM5 Fabs reformatted as human IgG₁ molecules. Each antibody was titrated against 293-huVSTM5, or 293-MT control cells. The bottom plot (labelled mAb binding vs. 293-muVSTM5) is an exception, showing binding against the 293-muVSTM5 (cell line expressing the mouse VSTM5 antigen). The mouse VSTM5 cross reactive antibodies 50-01.B01 and 53-01.B11 are indicated. The other mAbs do not bind 293-muVSTM5 cells and are indicates by open black squares. *Note the difference in axis size for mAb 52-01.A07, which accounts for the much lower binding activity.

Table 2 presents a summary of FACS binding results for human anti-VSTM5 IgGs. Binding activity of the anti-VSTM5 mAbs is indicated for both 293-huVSTM5 and 293-muVSTM5. Columns present relative binding strength for each mAb, based on the ratio of 293-huVSTM5 or 293-muVSTM5 cell binding/293-MT control cell binding at a mAb concentration of 22 nM. Ratio>2000 (++++), ratio 850-2000 (+++) ratio 400-850 (++), ratio 50-400 (+), ratio<50 (+/−), no binding (−). Cutoffs for each rank are based on the interquartile ranges of the data set.

TABLE 2

| | FACS binding | FACS binding |
|---|---|---|
| IgG name | human VSTM5 | mouse VSTM5 |
| 49-01.F05 | ++++ | — |
| 49-01.D06 | ++++ | — |
| 49-01.F01 | ++++ | — |
| 53-01.B11 | +++ | +++ |
| 50-01.E02 | +++ | — |
| 49-02.C11 | +++ | — |
| 50-01.D01 | ++ | — |
| 50-01.F03 | ++ | — |
| 47-01.D05 | ++ | — |
| 50-01.A04 | + | — |
| 50-01.B01 | + | + |
| 52-01.A07 | +/− | — |
| Human IgG₁ | — | — |
| isotype control | | |

Table 3 below presents results obtained in various characterization assays for human anti-VSTM5 IgGs.

| | | | VSTM5 | | |
|---|---|---|---|---|---|
| | | Co- | Monomer | | Mouse |
| | Bead | Culture | Binding | Epitope | VSTM5 |
| mAB | Assay | Assay | (KD) | Bin | Binding |
| 49-01.F01 | Neg. | Neg. | 2 × 10⁻⁹ M | 1 | No |
| 50-01.F03 | Borderline | Neg. | Unable to | Unable to | No |
| | | | Determine | Determine | |
| 50-01.E02 | Pos. | Neg. | Unable to | 2 | No |
| | | | Determine | | |
| 50-01.A04 | Pos. | Neg. | 5.28 × 10−8M | 4 | No |
| 53-01.B11 | Pos. | Pos. | Unable to | 5 | Yes |
| | | | Determine | | |
| 47-01.D05 | Neg. | Neg. | 6.07 × 10−8M | 3 | No |
| 49-01.F05 | Neg. | Pos. | 1.90 × 10−7M | 2 | No |
| 50-01.B01 | Neg. | Neg. | 6.57 × 10−7M | Unable to | Yes |
| | | | | Determine | |

Review

This Example relates to the isolation of different anti-VSTM5 antibodies, many of which bind VSTM5 with high affinity, and a number of which were shown herein to possess immunomodulatory properties, i.e., they modulate, e.g., inhibit or neutralize the suppressive effects of VSTM5 on immunity. It is anticipated that similar or different immunization and antibody selection methods may be used to derive other anti-VSTM5 antibodies, e.g., human or humanized anti-VSTM5 antibodies and antigen-binding fragments that modulate VSTM5 and which potentially may be used in human immunotherapy.

The Fab pool from panning experiments B, C and D obtained by the methods disclosed herein, when screened by ELISA, yielded reasonable hit rates (˜17-48%) and Fab sequence diversity (30-33%). Accordingly, these antibodies and methods may be used in further screening experiments and should give rise to antibodies or derivatives or optimized forms thereof, that potentially may be used in human or animal therapy.

In particular, 46 unique Fabs were identified based on their ability to bind VSTM5 in at least one assay. A subset of 24 was demonstrated to be reactive against the cell-surface form of human VSTM5, which is a pre-requisite for these antibodies to potentially possess the desired immunomodulatory activity. Twenty-three VSTM5 binding Fabs were reformatted and produced as IgG molecules. Of these, to date fifteen have been expressed and purified, with at least ten showing robust binding to the HEK293 human VSTM5 expressing cell line. Of these fifteen, two IgGs are still being characterized to confirm FACS binding activity (54-01.A04 and 52-01.A07). Antibody 53-01.B11 shows strong cross-reactivity against the mouse form of VSTM5.

The remaining Fabs may be converted into IgG antibodies by analogous methods and similarly assessed for their ability to antagonize or agonize the effects of VSTM5 on immunity as disclosed herein, especially VSTM5's suppressive effects on T and NK cell activity, and particularly on T cell and NK cell-mediated cytotoxicity and the production of proinflammatory cytokines.

Moreover, the subject methods may be conducted on a large scale in order to increase the number and diversity of human or murine VSTM5 binding antibodies. In addition antibody diversity may be increased by using other VSTM5 antigens, e.g., monomeric versions of human or murine VSTM5 or fragments or variants thereof.

In addition, this Example supports the potential production of optimized forms of the exemplified anti-VSTM5 antibodies. For example and without limitation, optionally sequencing of the subject anti-VSTM5 antibodies and affinity maturation are performed to yield anti-VSTM5 antibodies of even better affinities than those exemplified herein. Also, these antibodies may optionally be rendered more “human-like” by the incorporation of residues that are more common at specific sites in endogenous human antibodies.

Also, the exemplified anti-VSTM5 antibody sequences may optionally be fused to desired human constant or Fc regions, which constant or Fc regions potentially may be mutated to alter effector functions or half-life. Moreover, the exemplified anti-VSTM5 antibody sequences may optionally be attached to other targeting moieties such as receptors expressed on desired target cells or these antibody sequences may be used to obtain bispecific antibodies that bind VSTM5 and another desired antigen. Further, as disclosed in the Detailed Description, these antibodies or fragments thereof may be attached to desired effector molecules such as therapeutic or diagnostic agents.

### Example 13

### Monoclonal Antibody Sequencing

DNA encoding the heavy and light chain variable regions of each antibody was submitted to Elim Biopharmaceuticals (Hayward, Calif., USA) for sequencing using an ABI 3730x1 sequencer (Life Technologies). The resulting sequences were analyzed using the SeqAgent software package (XOMA Corporation, Berkeley, Calif., USA).

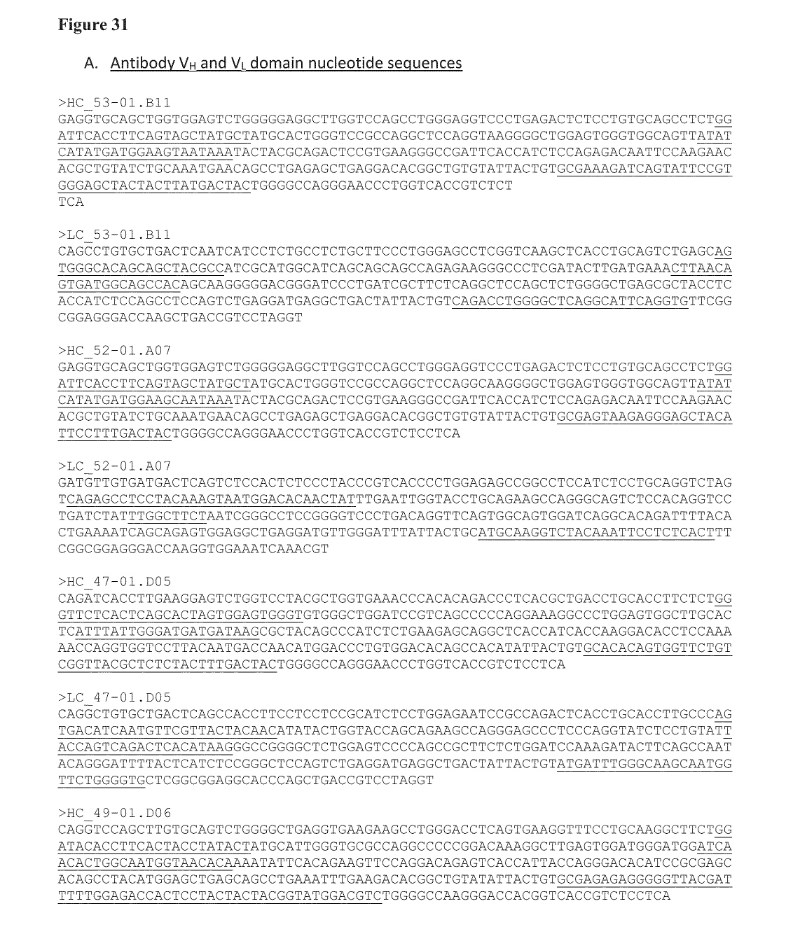

The DNA sequences of the heavy chain of the 47-01.D05; 49-01.D06; 49-01.F05; 49-02.C11; 49-01.F01; 50-01.A04; 50-01.B01; 50-01.E02; 50-01.F03; 50-01.D01; 52-01.A07; and 53-01.B11 antibodies are shown in SEQ ID NOs: 161, 163, 167, 169, 165, 171, 173, 177, 179, 175, 159 and 157, respectively, and in FIG. 31A.

The amino acid sequences of the heavy chain of the 47-01.D05; 49-01.D06; 49-01.F05; 49-02.C11; 49-01.F01; 50-01.A04; 50-01.801; 50-01.E02; 50-01.F03; 50-01.D01; 52-01.A07; and 53-01.B11 antibodies are shown in SEQ ID NOs: 257, 259, 263, 265, 261, 267, 269, 273, 275, 271, 255, 253, respectively, and in FIG. 31B.

The DNA sequences of the light chain of the 47-01.D05; 49-01.D06; 49-01.F05; 49-02.011; 49-01.F01; 50-01.A04; 50-01.B01; 50-01.E02; 50-01.F03; 50-01.D01; 52-01.A07; and 53-01.B11 antibodies are shown in SEQ ID NOs: 162, 164, 168, 170, 166, 172, 174, 178, 180, 176, 160, 158, respectively, and in FIG. 31A.

The amino acid sequences of the light chain of the 47-01.D05; 49-01.D06; 49-01.F05; 49-02.C11; 49-01.F01; 50-01.A04; 50-01.B01; 50-01.E02; 50-01.F03; 50-01.D01; 52-01.A07; and 53-01.B11 antibodies are shown in SEQ ID NOs: 258, 260, 264, 266, 262, 268, 270, 274, 276, 272, 256, 254, respectively, and in FIG. 31B.

The sequences of CDR1, CDR2, CDR3 in FIGS. 31A-B are underlined. “HC” corresponds to heavy chain; “LC” corresponds to light chain.

The nucleic acid sequences of the 47-01.D05; 49-01.D06; 49-01.F05; 49-02.C11; 49-01.F01; 50-01.A04; 50-01.B01; 50-01.E02; 50-01.F03; 50-01.D01; 52-01.A07; and 53-01.B11 antibodies heavy chain CDR1, CDR2, CDR3 are set forth in SEQ ID NOs: 193-195, 199-201, 211-213, 217-219, 205-207, 223-225, 229-231, 241-243, 247-249, 235-237, 187-189 and 181-183, respectively. The corresponding amino acid sequences of the 47-01.D05; 49-01.D06; 49-01.F05; 49-02.C11; 49-01.F01; 50-01.A04; 50-01.B01; 50-01.E02; 50-01.F03; 50-01.D01; 52-01.A07; and 53-01.B11 heavy chain CDR1, CDR2, CDR3 are set forth in SEQ ID NOs: 289-291, 295-297, 307-309, 313-315, 301-303, 319-321, 325-327, 337-339, 343-345, 331-333, 283-285, 277-279, respectively.

The nucleic acid sequences of the 47-01.D05; 49-01.D06; 49-01.F05; 49-02.C11; 49-01.F01; 50-01.A04; 50-01.B01; 50-01.E02; 50-01.F03; 50-01.D01; 52-01.A07; and 53-01.B11 antibodies light chain CDR1, CDR2, CDR3 are set forth in SEQ ID NOs: 196-198, 202-204, 214-216, 220-222, 208-210, 226-228, 232-234, 244-246, 250-252, 238-240, 190-192, 184-186, respectively. The corresponding amino acid sequences of the 47-01.D05; 49-01.D06; 49-01.F05; 49-02.C11; 49-01.F01; 50-01.A04; 50-01.B01; 50-01.E02; 50-01.F03; 50-01.D01; 52-01. A07; and 53-01.B11 antibodies light chain CDR1, CDR2, CDR3 are set forth in SEQ ID NOs: 292-294, 298-300, 310-312, 316-318, 304-306, 322-324, 328-330, 340-342, 346-348, 334-336, 286-288, 280-282, respectively.

Antibody V_{H} and V_{L} Domain Nucleotide Sequences

Complementarity Determining Regions (CDRs) Underlined

>HC_53-01.B11
(SEQ ID NO: 157)
GAGGTGCAGCTGGTGGAGTCTGGGGGAGGCTTGGTCCAGCCTGGGAGGTC
CCTGAGACTCTCCTGTGCAGCCTCTGGATTCACCTTCAGTAGCTATGCTA
TGCACTGGGTCCGCCAGGCTCCAGGTAAGGGGCTGGAGTGGGTGGCAGTT
ATATCATATGATGGAAGTAATAAATACTACGCAGACTCCGTGAAGGGCCG
ATTCACCATCTCCAGAGACAATTCCAAGAACACGCTGTATCTGCAAATGA
ACAGCCTGAGAGCTGAGGACACGGCTGTGTATTACTGTGCGAAAGATCAG
TATTCCGTGGGAGCTACTACTTATGACTACTGGGGCCAGGGAACCCTGGT
CACCGTCTCTTCA
>LC_53-01.B11
(SEQ ID NO: 158)
CAGCCTGTGCTGACTCAATCATCCTCTGCCTCTGCTTCCCTGGGAGCCTC
GGTCAAGCTCACCTGCAGTCTGAGCAGTGGGCACAGCAGCTACGCCATCG
CATGGCATCAGCAGCAGCCAGAGAAGGGCCCTCGATACTTGATGAAACTT
AACAGTGATGGCAGCCACAGCAAGGGGGACGGGATCCCTGATCGCTTCTC
AGGCTCCAGCTCTGGGGCTGAGCGCTACCTCACCATCTCCAGCCTCCAGT
CTGAGGATGAGGCTGACTATTACTGTCAGACCTGGGGCTCAGGCATTCAG
GTGTTCGGCGGAGGGACCAAGCTGACCGTCCTAGGT
>HC_52-01.A07
(SEQ ID NO: 159)
GAGGTGCAGCTGGTGGAGTCTGGGGGAGGCTTGGTCCAGCCTGGGAGGTC
CCTGAGACTCTCCTGTGCAGCCTCTGGATTCACCTTCAGTAGCTATGCTA
TGCACTGGGTCCGCCAGGCTCCAGGCAAGGGGCTGGAGTGGGTGGCAGTT
ATATCATATGATGGAAGCAATAAATACTACGCAGACTCCGTGAAGGGCCG
ATTCACCATCTCCAGAGACAATTCCAAGAACACGCTGTATCTGCAAATGA
ACAGCCTGAGAGCTGAGGACACGGCTGTGTATTACTGTGCGAGTAAGAGG
GAGCTACATTCCTTTGACTACTGGGGCCAGGGAACCCTGGTCACCGTCTC
CTCA
>LC_52-01.A07
(SEQ ID NO: 160)
GATGTTGTGATGACTCAGTCTCCACTCTCCCTACCCGTCACCCCTGGAGA
GCCGGCCTCCATCTCCTGCAGGTCTAGTCAGAGCCTCCTACAAAGTAATG
GACACAACTATTTGAATTGGTACCTGCAGAAGCCAGGGCAGTCTCCACAG
GTCCTGATCTATTTGGCTTCTAATCGGGCCTCCGGGGTCCCTGACAGGTT
CAGTGGCAGTGGATCAGGCACAGATTTTACACTGAAAATCAGCAGAGTGG
AGGCTGAGGATGTTGGGATTTATTACTGCATGCAAGGTCTACAAATTCCT
CTCACTTTCGGCGGAGGGACCAAGGTGGAAATCAAACGT
>HC_47-01.D05
(SEQ ID NO: 161)
CAGATCACCTTGAAGGAGTCTGGTCCTACGCTGGTGAAACCCACACAGAC
CCTCACGCTGACCTGCACCTTCTCTGGGTTCTCACTCAGCACTAGTGGAG
TGGGTGTGGGCTGGATCCGTCAGCCCCCAGGAAAGGCCCTGGAGTGGCTT
GCACTCATTTATTGGGATGATGATAAGCGCTACAGCCCATCTCTGAAGAG
CAGGCTCACCATCACCAAGGACACCTCCAAAAACCAGGTGGTCCTTACAA
TGACCAACATGGACCCTGTGGACACAGCCACATATTACTGTGCACACAGT
GGTTCTGTCGGTTACGCTCTCTACTTTGACTACTGGGGCCAGGGAACCCT
GGTCACCGTCTCCTCA
>LC_47-01.D05
(SEQ ID NO: 162)
CAGGCTGTGCTGACTCAGCCACCTTCCTCCTCCGCATCTCCTGGAGAATC
CGCCAGACTCACCTGCACCTTGCCCAGTGACATCAATGTTCGTTACTACA
ACATATACTGGTACCAGCAGAAGCCAGGGAGCCCTCCCAGGTATCTCCTG
TATTACCAGTCAGACTCACATAAGGGCCGGGGCTCTGGAGTCCCCAGCCG
CTTCTCTGGATCCAAAGATACTTCAGCCAATACAGGGATTTTACTCATCT
CCGGGCTCCAGTCTGAGGATGAGGCTGACTATTACTGTATGATTTGGGCA
AGCAATGGTTCTGGGGTGCTCGGCGGAGGCACCCAGCTGACCGTCCTAGG
T
>HC_49-01.D06
(SEQ ID NO: 163)
CAGGTCCAGCTTGTGCAGTCTGGGGCTGAGGTGAAGAAGCCTGGGACCTC
AGTGAAGGTTTCCTGCAAGGCTTCTGGATACACCTTCACTACCTATACTA
TGCATTGGGTGCGCCAGGCCCCCGGACAAAGGCTTGAGTGGATGGGATGG
ATCAACACTGGCAATGGTAACACAAAATATTCACAGAAGTTCCAGGACAG
AGTCACCATTACCAGGGACACATCCGCGAGCACAGCCTACATGGAGCTGA
GCAGCCTGAAATTTGAAGACACGGCTGTATATTACTGTGCGAGAGAGGGG
GTTACGATTTTTGGAGACCACTCCTACTACTACGGTATGGACGTCTGGGG
CCAAGGGACCACGGTCACCGTCTCCTCA
>LC_49-01.D06
(SEQ ID NO: 164)
CAGCCTGTGCTGACTCAATCATCCTCTGCCTCTGCTTCCCTGGGATCCTC
GGTCAAGCTCACCTGCACTCTGAGCAGTGGGCACAGTAGCTACATCATCG
CATGGCATCAGCAGCAGCCAGGGAAGGCCCCTCGATACTTGATGAAGCTT
GAAGGTAGTGGAAGCTACAACAAGGGGAGCGGACTTCCTGATCGCTTCTC
AGGCTCCAGCTCTGGGGCTGACCGCTACCTCACCATCTCCAACCTCCAGT
CTGAGGATGAGGCTGATTATTACTGTGAGACCTGGGACGGTAACACTTGG
GTGTTCGGCGGAGGCACCCAGCTGACCGTCCTAGGT
>HC_49-01.F01
(SEQ ID NO: 165)
GAGGTGCAGCTGGTGGAGTCTGGGGGAGGTGTGGTCAAGCCGGGGGAGTC
CCTGAGACTCTTATGTACAGCCTCTGGATTGTCCTTCAATAAATACAGCA
TAAATTGGGTCCGCCAGGCTCCAGGGGGGGGGCTTGAGTGGGTCTCATCG
ATTGAAAGTGGTAGTGGACATATATATTACGCAGACTCACTGGAGGGCCG
CTTCACCATCTCCAGAGATAACGCCAAGAACTCCGTGACTCTGGAAATGA
ACAGCCTGAGAGTCGAGGACACGGCTCTTTATTACTGTGTCTCGGGGCCG
GAAGACAAGTGGTTGTTGCAGCTTTACTTTGAGTCCTGGGGCCAGGGAAC
CCTGGTCACCGTCTCCTCA
>LC_49-01.F01
(SEQ ID NO: 166)
GAAATTGTGTTGACGCAGTCTCCAGCCACCCTGTCTTTGTCTCCAGGGGA
AAGAGCCACCCTCTCCTGCAGGACCAGTCAGAGTTCTCCCAGCGACAACT
TAGCCTGGTATCAGCACAAACCTGGCCAGGCTCCGAGGCTCCTCATCTAC
GGTGGTTCCAACAGGGCCACTGGCATCCCAGCCAGGTTCAGTGGCAGTGG
GTCTGGGACAGACTTCACTCTCACCATCAGCAGCCTAGAGCCTGAAGATT
TTGCAGTGTATTACTGTCAGCAGTATGGTAGCTCACCGCTCACTTTCGGC
GGAGGGACCAAAGTGGATATCAAACGT
>HC_49-01.F05
(SEQ ID NO: 167)
CAGGTGCAGCTACAGCAGTGGGGCGCAGGACTGTTGAAGCCTTCGGAGAC
CCTGTCCCTCACCTGCGCTGTCTATGGTGGGTCCTTCAGTGGTTACTACT
GGAGCTGGATCCGCCAGCCCCCAGGGAAGGGGCTGGAGTGGATTGGGGAA
ATCAATCATAGTGGAAGCACCAACTACAACCCGTCCCTCAAGAGTCGAGT
CACCATATCAGTAGACACGTCCAAGAACCAGTTCTCCCTGAAGCTGAGCT
CTGTGACCGCCGCGGACACGGCTGTGTATTACTGTGCGAGAGGCTGGCGA
GGTGGTAGCTTTATGGACGTCTGGGGCCAAGGGACCACGGTCACCGTCTC
CTCA
>LC_49-01.F05
(SEQ ID NO: 168)
CAGTCTGCCCTGACTCAGCCTCGCTCAGTGTCCGGGTCTCCTGGACAGTC
AGTCACCATCTCCTGCACTGGAACCAGCAGTGATGTTGGTGGTTATAACT
ATGTCTCCTGGTACCAACAGCACCCAGGCAAAGCCCCCAAACTCATGATT
TATGATGTCATTAAGCGGCCCTCAGGGGTCCCTGATCGCTTCTCTGGCTC
CAGGTCTGGCAACACGGCCTCCCTGACCATCTCTGGGCTCCAGGCTGAAG
ATGAGGCTGATTATTATTGCTGCTCATATGCAGGCACCCATTGGGTGTTC
GGCGGAGGGACCAAGCTCACCGTCCTAGGT
>HC_49-02.C11
(SEQ ID NO: 169)
GAGGTGCAGCTGGTGGAGTCTGGGGGAGGCTTGGTCCAGCCTGGGGGGTC
CCTGAGACTCTCCTGTGCAGCCTCTGGATTCACCTTTAGCAGCTATGCCA
TGACCTGGGTCCGCCAGGCTCCAGGGAAGGGGCTGGAGTGGGTCTCAGTT
ATTAGTGGTGGTGGTGGTACCACATACTACGCAGACTCCGTGAAGGGCCG
CTTCACCATCTCCAGAGACAATTCCAAGAACACTCTGTATCTGCAAATGA
GCAGCCTGAGAGCCGAGGACACGGCCGTATATTACTGTGCGAAAGGGTAT
AGCAGTGGCTGGCCCTACTACTTTGACTACTGGGGCCAGGGAACCCTGGT
CACCGTCTCCTCA
>LC_49-02.C11
(SEQ ID NO: 170)
GAAATTGTGTTGACGCAGTCTCCAGGCACCCTGTCTTTGTCTCCAGGGGA
AAGAGCCACCCTCTCCTGCAGGGCCAGTCAGAGTGGCGTCACCAATTTCT
TAGCCTGGTATCAGCAGAAACCTGGCCAGGCTCCCAGGCTCCTCATCTAT
GCTACTTCCAGCAGGGCCACTGGCATCCCAGACAGGTTCAGTGGCAGTGC
GTCTGGGACAGACTTCACTCTCACCATCACCAGACTGGAGCCTGAAGATT
TTGCAGTTTATTTCTGCCAGCAATATGCTTCCTCACCGCTCACTTTCGGC
GGAGGGACCAAAGTGGAGATCAAACGT
>HC_50-01.A04
(SEQ ID NO: 171)
CAGGTGCAGCTGGTGCAATCTGGAGCAGAGGTGAAAAAGCCCGGGGAGTC
TCTGAAGATCTCCTGTAAGGGTTCTGGATACGACTTTAATAATTACTGGA
TCGGCTGGGTGCGCCAGACGCCCGAGAAGGGCCTGGAGTGGATGGGGATC
GTCTATCCTGGTGACCATCCTGGTGACTATCATATCAGATATGGCCCGTC
CTTCCAAGGCCAGGTCACCATCTCAGCCGACAGGTCCATCACCACCGCCT
ACCTACAGTGGAGAAACCTGAAGGCCTCGGACACCGCCATGTATTACTGT
GCGAGACTAGGAAGCAGTAAAGACCTTGACTACTGGGGCCAGGGAACCCT
GGTCACCGTCTCCTCA
>LC_50-01.A04
(SEQ ID NO: 172)
GAAATTGTGTTGACGCAGTCTCCAGCCACCCTGTCTTTGTCTCCAGGGGA
AAGAGCCACCCTCTCCTGCAGGGCCAGTCAGAGTGTTAGCGGACACTTAG
ACTGGTACCAACAGAAACCTGGCCAGTCTCCCAGGCTCCTCATCTATGAT
GCATCCAACAGGGCCACTGGCATCCCAGCCAGGTTCAGTGGCAGTGGGTC
TGGGACAGACTTCTCTCTCACCATCAGCAGCCTAGAGCCTGAAGATTTTG
CAGTTTATTACTGTCAGCACCGTAGCAACTGGCCGTGGACGTTCGGCCAA
GGGACCAAGGTGGAAATCAAACGT
>HC_50-01.B01
(SEQ ID NO: 173)
CAGGTGCAGCTGCAGGAGTCGGGCCCAGGACTGATGAAGCCTTCACAGAC
CCTGTCCCTCACCTGCACTGTCTCTGGTGACTCCATCACCAGAGGTAGCA
GTTACTGGAGTTGGATCCGGCAGTCCGACGGGAAGGGACTGGAGTGGATT
GGGCACATCTATAGTGGAGGGGACACCGACTACAATCCCGCCCTCAAGAG
TCGAGTCACTATATCAGCTGACGCGTCCAGGGGCCAGTTTTTGTTGAGAT
TGACCTCAATGACCGCCGCAGACACGGCCGTTTATTACTGTGCGAGAGAT
CGTGGAGCATACGGTATGGACGTCTGGGGCCAAGGGACCACGGTCACCGT
CTCCTCA
>LC_50-01.B01
(SEQ ID NO: 174)
CAGCCTGTGCTGACTCAATCATCCTCTGCCTCTGCTTCCCTGGGAGCCTC
GGTCAAGCTCACCTGCACTCTGAGCAGTGAACACGACAGATATGCCATCG
CATGGCTTCAACAGAAGCCAGAGAAGGGTCCTCGCTACTTGATGAAGGTT
AACAGTGATGGCAGCCACAGGAAGGGGGACGGGATCCCTGATCGCTTCTC
AGGCTCCAGTTCTGGGGCTGAGCGCTACCTCACCATCTCCAGACTCCAGT
CTGAGGATGAGGCTGACTATTACTGTCAGACCTGGGGCATTGGCATTAGG
GTGTTCGGCGGTGGCACCCAGCTGACCGTCCTAGGT
>HC_50-01.001
(SEQ ID NO: 175)
CAGGTGCAGCTGGTGGAGTCTGGGGGAGGCGTGGTCCAGCCTGGGAGGTC
CCGCAGACTCTCCTGTGCAGCGTCTGGATTCGCCTTCCGTAATTATGGCA
TGCACTGGGTCCGCCAGGCTCCAGGCAAGGGGCTGGAGTGGGTGGCATTC
ATATCACAAAACGGAGGTAAGAAATATTATGCAGACTCCGTGACGGGCCG
ATTCACCGTCTCCAGAGACAATTCCAAGAACACGTTGTATCTGCAAATAA
ACAGCCTGACAACTGACGACACGGCTGTGTTTTACTGTGCGAGGTCGGGG
AGCGGGTCATGGGGCTACAGTGACTTCCCCGGACCCTTTGACCACTGGGG
CCAGGGATCCCTGGTCACTGTCTCCTCA
>LC_50-01.001
(SEQ ID NO: 176)
GAAATTGTGTTGACGCAGTCTCCAGGCACCCTGTCTTTGTCTCCAGGGGA
AAGAGCCACCCTCTCCTGCAGGGCCAGTCAGAATATTTTCATCAGCTTCT
TAGCCTGGTACCAGCAGAAACCTGGCCAGGCTCCCAGGCTCCTCCTCTAT
GGTGCTTCCAACAGGGCCACTGGCATCCCAGACAGGTTCAGTGGCAGTGG
GTCTGGGACAGACTTCACTCTCACCATCAGCAGACTGGAGCCTGAAGATT
TTGCAGTGTATTACTGTCAGCAGTATGGTAGCTCCCCGCTCACTTTCGGC
GGAGGGACCAAAGTGGAGATCAAACGT
>HC_50-01.E02
(SEQ ID NO: 177)
CAGGTGCAGCTGGTGGAGTCTGGGGGAGGCGTGGTCCAGCCTGGGGGGTC
CTTGAAACTCTCCTGTGTAGCCTCTGGATTCACCTTTAGCGCCTATGCCA
TGAACTGGGTCCGCCTGGTTCCAGGTAAGGGGCTGGAGTGGGTCTCAGGT
ATTAGTGGCAATGGCTATTCCACATTCTACCCAGACTCCGTGCAGGGCCG
ATTCACCGTCTCCAGAGACAATTCCAAGAACACGTTGTTTCTGCAAATTG
ATAGGCTGACAGGCGGGGACACGGCCATATACTACTGTGCGAAGGTACAG
ACTACGGTTATTACTCCTTTTCAAAACTGGGGCCAGGGAACCCTGGTCAC
CGTCTCTTCA
>LC_50-01.E02
(SEQ ID NO: 178)
GAAATTGTGTTGACGCAGTCTCCAGGCACCCTGTCTTTGTCTCCAGGGGA
AAGAGCCACCCTCTCCTGCAGGGCCAGTCAGAATATTGGCAGCAACTTCT
TAGCCTGGTACCAGCAAAAACCTGGCCAGGCTCCCAGGCTCCTCATCTAT
GGTGCGTCCACCAGGGCCAATGGCATCCCAGACAGGTTCAGTGGCAGTAA
GTCTGAGACAGACTTCACTCTCACCATCAGCAGACTGGAGCCTGAAGATT
TTGCAGTGTATTACTGTCAGCAGTATGATAACTCACCGTACACTTTTGGC
CAGGGGACCAAGCTGGAGATCAAACGT
>HC_50-01.F03
(SEQ ID NO: 179)
GAGGTGCAGCTGGTGGAGATTGGAGGAGGCTTGATCCAGCCTGGGAGGTC
CCTGAGACTCTCCTGTGCAGCCTCTGGATTCACCTTCAGTAGCTATGCTA
TGCACTGGGTCCGCCAGGCTCCAGGCAAGGGGCTGGAGTGGGTGGCGGTT
ATATCATATGATGGAAGCAAAAAATACTACGCAGACTCCGTGAAGGGCCG
ATTCACCATCTCCAGAGACAATTCCAAGAACACGGTGCATCTGCAAATGA
ACAGCCTGAGAGTCGAGGATACGGCTGTCTATTACTGTGCGCTCTTGTCC
CGTCCACACTACGGTTTGGACGTCTGGGGCCAAGGGACCACGGTCACCGT
CTCCTCA
>LC_50-01.F03
(SEQ ID NO: 180)
CAGTCTGTGCTGACGCAGCCGCCCTCAGTGTCTGCGGCCCCAGGACAGAA
GGTCACCATCTCCTGCTCTGGAAGTCGCTCCAACGTTGGGGGTAATTTTC
TTTCCTGGTACCAACACGTCCCAGGAACACCCCCCCAACTCCTCATTTAT
GACAATTATAAGCGACCCTCAGAGATACCTGACCGATTCTCGGGCTCCAA
GTCTGGCACGTCAGCCACCCTGGACATCACCGGACTCCAGACTGGGGACG
AGGCCGATTATTACTGCGGAACATGGGATAGCAGCCTGAGTTCTTGGGTG
TTCGGCGGAGGCACCCAGCTGACCGTCCTAGGT

Antibody V_{H} and V_{L} Domain Amino Acid Sequences

Complementarity Determining Regions (CDRs) Underlined

>HC_53-01.B11
(SEQ ID NO: 253)
EVQLVESGGGLVQPGRSLRLSCAASGFTFSSYAMHWVRQAPGKGLEWVAV
ISYDGSNKYYADSVKGRFTISRDNSKNTLYLQMNSLRAEDTAVYYCAKDQ
YSVGATTYDYWGQGTLVTVSS
>LC_53-01.B11
(SEQ ID NO: 254)
QPVLTQSSSASASLGASVKLTCSLSSGHSSYAIAWHQQQPEKGPRYLMKL
NSDGSHSKGDGIPDRFSGSSSGAERYLTISSLQSEDEADYYCQTWGSGIQ
VFGGGTKLTVLG
>HC_52-01.A07
(SEQ ID NO: 255)
EVQLVESGGGLVQPGRSLRLSCAASGFTFSSYAMHWVRQAPGKGLEWVAV
ISYDGSNKYYADSVKGRFTISRDNSKNTLYLQMNSLRAEDTAVYYCASKR
ELHSFDYWGQGTLVTVSS
>LC_52-01.A07
(SEQ ID NO: 256)
DVVMTQSPLSLPVTPGEPASISCRSSQSLLQSNGHNYLNWYLQKPGQSPQ
VLIYLASNRASGVPDRFSGSGSGTDFTLKISRVEAEDVGIYYCMQGLQIP
LTFGGGTKVEIKR
>HC_47-01.D05
(SEQ ID NO: 257)
QITLKESGPTLVKPTQTLTLTCTFSGFSLSTSGVGVGWIRQPPGKALEWL
ALIYWDDDKRYSPSLKSRLTITKDTSKNQVVLTMTNMDPVDTATYYCAHS
GSVGYALYFDYWGQGTLVTVSS
>LC_47-01.D05
(SEQ ID NO: 258)
QAVLTQPPSSSASPGESARLTCTLPSDINVRYYNIYWYQQKPGSPPRYLL
YYQSDSHKGRGSGVPSRFSGSKDTSANTGILLISGLQSEDEADYYCMIWA
SNGSGVLGGGTQLTVLG
>HC_49-01.D06
(SEQ ID NO: 259)
QVQLVQSGAEVKKPGTSVKVSCKASGYTFTTYTMHWVRQAPGQRLEWMGW
INTGNGNTKYSQKFQDRVTITRDTSASTAYMELSSLKFEDTAVYYCAREG
VTIFGDHSYYYGMDVWGQGTTVTVSS
>LC_49-01.D06
(SEQ ID NO: 260)
QPVLTQSSSASASLGSSVKLTCTLSSGHSSYIIAWHQQQPGKAPRYLMKL
EGSGSYNKGSGLPDRFSGSSSGADRYLTISNLQSEDEADYYCETWDGNTW
VFGGGTQLTVLG
>HC_49-01.F01
(SEQ ID NO: 261)
EVQLVESGGGVVKPGESLRLLCTASGLSFNKYSINWVRQAPGGGLEWVSS
IESGSGHIYYADSLEGRFTISRDNAKNSVTLEMNSLRVEDTALYYCVSGP
EDKWLLQLYFESWGQGTLVTVSS
>LC_49-01.F01
(SEQ ID NO: 262)
EIVLTQSPATLSLSPGERATLSCRTSQSSPSDNLAWYQHKPGQAPRLLIY
GGSNRATGIPARFSGSGSGTDFTLTISSLEPEDFAVYYCQQYGSSPLTFG
GGTKVDIKR
>HC_49-01.F05
(SEQ ID NO: 263)
QVQLQQWGAGLLKPSETLSLTCAVYGGSFSGYYWSWIRQPPGKGLEWIGE
INHSGSTNYNPSLKSRVTISVDTSKNQFSLKLSSVTAADTAVYYCARGWR
GGSFMDVWGQGTTVTVSS
>LC_49-01.F05
(SEQ ID NO: 264)
QSALTQPRSVSGSPGQSVTISCTGTSSDVGGYNYVSWYQQHPGKAPKLMI
YDVIKRPSGVPDRFSGSRSGNTASLTISGLQAEDEADYYCCSYAGTHWVF
GGGTKLTVLG
>HC_49-02.C11
(SEQ ID NO: 265)
EVQLVESGGGLVQPGGSLRLSCAASGFTFSSYAMTWVRQAPGKGLEWVSV
ISGGGGTTYYADSVKGRFTISRDNSKNTLYLQMSSLRAEDTAVYYCAKGY
SSGWPYYFDYWGQGTLVTVSS
>LC_49-02.C11
(SEQ ID NO: 266)
EIVLTQSPGTLSLSPGERATLSCRASQSGVTNFLAWYQQKPGQAPRLLIY
ATSSRATGIPDRFSGSASGTDFTLTITRLEPEDFAVYFCQQYASSPLTFG
GGTKVEIKR
>HC_50-01.A04
(SEQ ID NO: 267)
QVQLVQSGAEVKKPGESLKISCKGSGYDFNNYWIGWVRQTPEKGLEWMGI
VYPGDHPGDYHIRYGPSFQGQVTISADRSITTAYLQWRNLKASDTAMYYC
ARLGSSKDLDYWGQGTLVTVSS
>LC_50-01.A04
(SEQ ID NO: 268)
EIVLTQSPATLSLSPGERATLSCRASQSVSGHLDWYQQKPGQSPRLLIYD
ASNRATGIPARFSGSGSGTDFSLTISSLEPEDFAVYYCQHRSNWPWTFGQ
GTKVEIKR
>HC_50-01.B01
(SEQ ID NO: 269)
QVQLQESGPGLMKPSQTLSLTCTVSGDSITRGSSYWSWIRQSDGKGLEWI
GHIYSGGDTDYNPALKSRVTISADASRGQFLLRLTSMTAADTAVYYCARD
RGAYGMDVWGQGTTVTVSS
>LC_50-01.B01
(SEQ ID NO: 270)
QPVLTQSSSASASLGASVKLTCTLSSEHDRYAIAWLQQKPEKGPRYLMKV
NSDGSHRKGDGIPDRFSGSSSGAERYLTISRLQSEDEADYYCQTWGIGIR
VFGGGTQLTVLG
>HC_50-01.D01
(SEQ ID NO: 271)
QVQLVESGGGVVQPGRSRRLSCAASGFAFRNYGMHWVRQAPGKGLEWVAF
ISQNGGKKYYADSVTGRFTVSRDNSKNTLYLQINSLTTDDTAVFYCARSG
SGSWGYSDFPGPFDHWGQGSLVTVSS
>LC_50-01.D01
(SEQ ID NO: 272)
EIVLTQSPGTLSLSPGERATLSCRASQNIFISFLAWYQQKPGQAPRLLLY
GASNRATGIPDRFSGSGSGTDFTLTISRLEPEDFAVYYCQQYGSSPLTFG
GGTKVEIKR
>HC_50-01.E02
(SEQ ID NO: 273)
QVQLVESGGGVVQPGGSLKLSCVASGFTFSAYAMNWVRLVPGKGLEWVSG
ISGNGYSTFYPDSVQGRFTVSRDNSKNTLFLQIDRLTGGDTAIYYCAKVQ
TTVITPFQNWGQGTLVTVSS
>LC_50-01.E02
(SEQ ID NO: 274)
EIVLTQSPGTLSLSPGERATLSCRASQNIGSNFLAWYQQKPGQAPRLLIY
GASTRANGIPDRFSGSKSETDFTLTISRLEPEDFAVYYCQQYDNSPYTFG
QGTKLEIKR
>HC_50-01.F03
(SEQ ID NO: 275)
EVQLVEIGGGLIQPGRSLRLSCAASGFTFSSYAMHWVRQAPGKGLEWVAV
ISYDGSKKYYADSVKGRFTISRDNSKNTVHLQMNSLRVEDTAVYYCALLS
RPHYGLDVWGQGTTVTVSS
>LC_50-01.F03
(SEQ ID NO: 276)
QSVLTQPPSVSAAPGQKVTISCSGSRSNVGGNFLSWYQHVPGTPPQLLIY
DNYKRPSEIPDRFSGSKSGTSATLDITGLQTGDEADYYCGTWDSSLSSWV
FGGGTQLTVLG

### Example 14

### Development of Fully Human Anti-VSTM5 Antibodies by Other Methods

Generation of Human Monoclonal Antibodies Against VSTM5 Antigen

Fusion proteins composed of the extracellular domain of the VSTM5 linked to a mouse IgG2 Fc polypeptide are generated by standard recombinant methods and used as antigen for immunization.

Transgenic HuMab Mouse.

Fully human monoclonal antibodies to VSTM5 are prepared using mice from the HCo7 strain of the transgenic HuMab Mouse™ which expresses human antibody genes. In this mouse strain, the endogenous mouse kappa light chain gene has been homozygously disrupted as described in Chen et al. (1993) EMBO J. 12:811-820 and the endogenous mouse heavy chain gene has been homozygously disrupted as described in Example 1 of PCT Publication WO 01/09187. Furthermore, this mouse strain carries a human kappa light chain transgene, KCo5, as described in Fishwild et al. (1996) Nature Biotechnology 14:845-851, and a human heavy chain transgene, HCo7, as described in U.S. Pat. Nos. 5,545,806; 5,625,825; and 5,545,807.

HuMab Immunizations:

To generate fully human monoclonal antibodies to VSTM5, mice of the HCo7 HuMab Mouse strain can be immunized with purified recombinant VSTM5 fusion protein derived from mammalian cells that are transfected with an expression vector containing the gene encoding the fusion protein. General immunization schemes for the HuMab Mouse are described in Lonberg, N. et al (1994) Nature 368(6474): 856-859; Fishwild, D. et al. (1996) Nature Biotechnology 14: 845-851 and PCT Publication WO 98/24884. The mice are 6-16 weeks of age upon the first infusion of antigen. A purified recombinant VSTM5 antigen preparation (5-50 μg, purified from transfected mammalian cells expressing VSTM5 fusion protein) is used to immunize the HuMab mice intraperitoneally.

Transgenic mice are immunized twice with antigen in complete Freund's adjuvant or Ribi adjuvant IP, followed by 3-21 days IP (up to a total of 11 immunizations) with the antigen in incomplete Freund's or Ribi adjuvant. The immune response is monitored by retroorbital bleeds. The plasma is screened by ELISA (as described below), and mice with sufficient titers of anti-VSTM5 human immunoglobulin are used for fusions. Mice are boosted intravenously with antigen 3 days before sacrifice and removal of the spleen.

Selection of HuMab Mice Producing Anti-VSTM5 Antibodies:

To select HuMab mice producing antibodies that bind VSTM5 sera from immunized mice is tested by a modified ELISA as originally described by Fishwild, D. et al. (1996). Briefly, microtiter plates are coated with purified recombinant VSTM5 fusion protein at 1-2 μg/ml in PBS, 50 μl/wells incubated 4° C. overnight then blocked with 200 μl/well of 5% BSA in PBS. Dilutions of plasma from VSTM5-immunized mice are added to each well and incubated for 1-2 hours at ambient temperature. The plates are washed with PBS/Tween and then incubated with a goat-anti-human kappa light chain polyclonal antibody conjugated with alkaline phosphatase for 1 hour at room temperature. After washing, the plates are developed with pNPP substrate and analyzed by spectrophotometer at OD 415-650. Mice that developed the highest titers of anti-VSTM5 antibodies are used for fusions. Fusions are performed as described below and hybridoma supernatants are tested for anti-VSTM5 activity by ELISA.

Generation of Hybridomas Producing Human Monoclonal Antibodies to VSTM5.

The mouse splenocytes, isolated from the HuMab mice, are fused with PEG to a mouse myeloma cell line based upon standard protocols. The resulting hybridomas are then screened for the production of antigen-specific antibodies. Single cell suspensions of splenic lymphocytes from immunized mice are fused to one-fourth the numbers of P3X63 Ag8.6.53 (ATCC CRL 1580) nonsecreting mouse myeloma cells with 50% PEG (Sigma). Cells are plated at approximately 1×10⁻⁵ /well in flat bottom microtiter plate, followed by about two week incubation in selective medium containing 10% fetal calf serum, supplemented with origen (IGEN) in RPMI, L-glutamine, sodium pyruvate, HEPES, penicillin, streptomycin, gentamycin, 1×HAT, and beta-mercaptoethanol. After 1-2 weeks, cells are cultured in medium in which the HAT is replaced with HT. Individual wells are then screened by ELISA (described above) for human anti-VSTM5 monoclonal IgG antibodies. Once extensive hybridoma growth occurred, medium is monitored usually after 10-14 days. The antibody secreting hybridomas are replated, screened again and, if still positive for human IgG, anti-VSTM5 monoclonal antibodies are subcloned at least twice by limiting dilution. The stable subclones are then cultured in vitro to generate small amounts of antibody in tissue culture medium for further characterization. The hybridoma clones are selected for further analysis.

Structural Characterization of Desired Anti-VSTM5 Human Monoclonal Antibodies

The cDNA sequences encoding the heavy and light chain variable regions of the obtained anti-VSTM5 monoclonal antibodies are obtained from the resultant hybridomas, respectively, using standard PCR techniques and are sequenced using standard DNA sequencing techniques.

The nucleotide and amino acid sequences of the heavy chain variable region and of the light chain variable region are identified. These sequences may be compared to known human germline immunoglobulin light and heavy chain sequences and the CDRs of each heavy and light of the obtained anti-VSTM5 sequences identified.

Characterization of Binding Specificity and Binding Kinetics of Anti-VSTM5 Human Monoclonal Antibodies

The binding affinity, binding kinetics, binding specificity, and cross-competition of anti-VSTM5 antibodies are examined by Biacore analysis. Also, binding specificity is examined by flow cytometry.

Binding Affinity and Kinetics

Anti-VSTM5 antibodies produced according to the invention are characterized for affinities and binding kinetics by Biacore analysis (Biacore AB, Uppsala, Sweden). Purified recombinant human VSTM5 fusion protein is covalently linked to a CM5 chip (carboxy methyl dextran coated chip) via primary amines, using standard amine coupling chemistry and kit provided by Biacore. Binding is measured by flowing the antibodies in HBS EP buffer (provided by Biacore AB) at a concentration of 267 nM at a flow rate of 50 μl/min. The antigen-association antibodies association kinetics is followed for 3 minutes and the dissociation kinetics is followed for 7 minutes. The association and dissociation curves are fit to a 1:1 Langmuir binding model using BIAevaluation software (Biacore AB). To minimize the effects of avidity in the estimation of the binding constants, only the initial segment of data corresponding to association and dissociation phases are used for fitting.

Epitope Mapping of Obtained Anti-VSTM5 Antibodies

Biacore is used to determine epitope grouping of anti-VSTM5 HuMAbs. Obtained anti-VSTM5 antibodies are used to map their epitopes on the VSTM5 antigen. These different antibodies are coated on three different surfaces of the same chip to 8000 RUs each. Dilutions of each of the mAbs are made, starting at 10 μg/mL and is incubated with Fc fused VSTM5 (50 nM) for one hour. The incubated complex is injected over all the three surfaces (and a blank surface) at the same time for 1.5 minutes at a flow rate of 20 .mu.L/min. Signal from each surface at end of 1.5 minutes, after subtraction of appropriate blanks, has been plotted against concentration of mAb in the complex. Upon analysis of the data, the anti-VSTM5 antibodies are categorized into different epitope groups depending on the epitope mapping results. The functional properties thereof are also compared.

Chinese hamster ovary (CHO) cell lines that express VSTM5 protein at the cell surface are developed and used to determine the specificity of the VSTM5 HuMAbs by flow cytometry. CHO cells are transfected with expression plasmids containing full length cDNA encoding a transmembrane forms of VSTM5 antigen or a variant thereof. The transfected proteins contained an epitope tag at the N-terminus are used for detection by an antibody specific for the epitope. Binding of a anti-VSTM5 MAb is assessed by incubating the transfected cells with each of the r VSTM5 Abs at a concentration of 10 μg/ml. The cells are washed and binding is detected with a FITC-labeled anti-human IgG Ab. A murine anti-epitope tag Ab, followed by labeled anti-murine IgG, is used as the positive control. Non-specific human and murine Abs are used as negative controls. The obtained data is used to assess the specificity of the HuMAbs for the VSTM5 antigen target.

### Example 15

### Expression of VSTM5 as Detected by Fully Human Anti-VSTM5 Antibodies on Human Peripheral Blood Leukocytes Isolated from Healthy Donors

The expression of VSTM5 polypeptide was further assayed on human peripheral blood leukocytes from healthy donors. Lymphocytes, granulocytes, platelets, monocytes, and CD11b^{low}CD14⁻ cells were examined. As discussed below, VSTM5 expression was observed on CD14⁺ monocytes, CD11b^{low}CD14⁻ cells, and to a lesser degree on eosinophils. No expression was observed on platelets, neutrophils, and lymphocytes. The Materials & Methods used in these assays are described below.

Materials and Methods

FACS Staining

Human peripheral blood was collected from 3 healthy donors. Red blood cells were lysed with RBC Lysis Buffer (eBiosciences Cat#00-4333-57) according to the manufacturer's protocol. Cells were incubated with 400 ug/ml human IgG (Jackson Immunoresearch #009-000-003) for 20 min on ice to block Fc receptors and then stained with cocktails consisting of Synagis® AF647, and anti-VSTM5 antibodies disclosed in Example 12, i.e., anti-VSTM5 AF647 (clone 44-2.E06 or 53-01.B11), anti-CD11b PE (Biolegend Cat#301305), anti-CD45 PerCp Cy5.5 (Biolegend #304207), anti-CD14 FITC (Biolegend cat#352610), or anti-CD41 PE (Biolegend Cat#303705) for 30 min on ice. All AF647 antibodies were conjugated at Compugen using Alexa Fluor® 647 Antibody Labeling Kit (Life Technologies Cat# A-20186) according to the manufacturer's protocol. Cells were washed twice with FACS Buffer (1% BSA, 0.1% Sodium Azide, PBS) and acquired on Intellicyte FACS machine. Data was analyzed by FlowJo and cell subsets defined as below: Monocytes: CD14⁺CD11b⁺, Neutrophils: CD11b⁺CD14^{low}, Lymphocytes FSC^{low}SSC^{lo}, Eosinophils SSC^{hi}, platelets: CD45⁻CD41⁺. Staining of anti-VSTM5 antibodies to these cells was compared to staining with Synagis.

Results

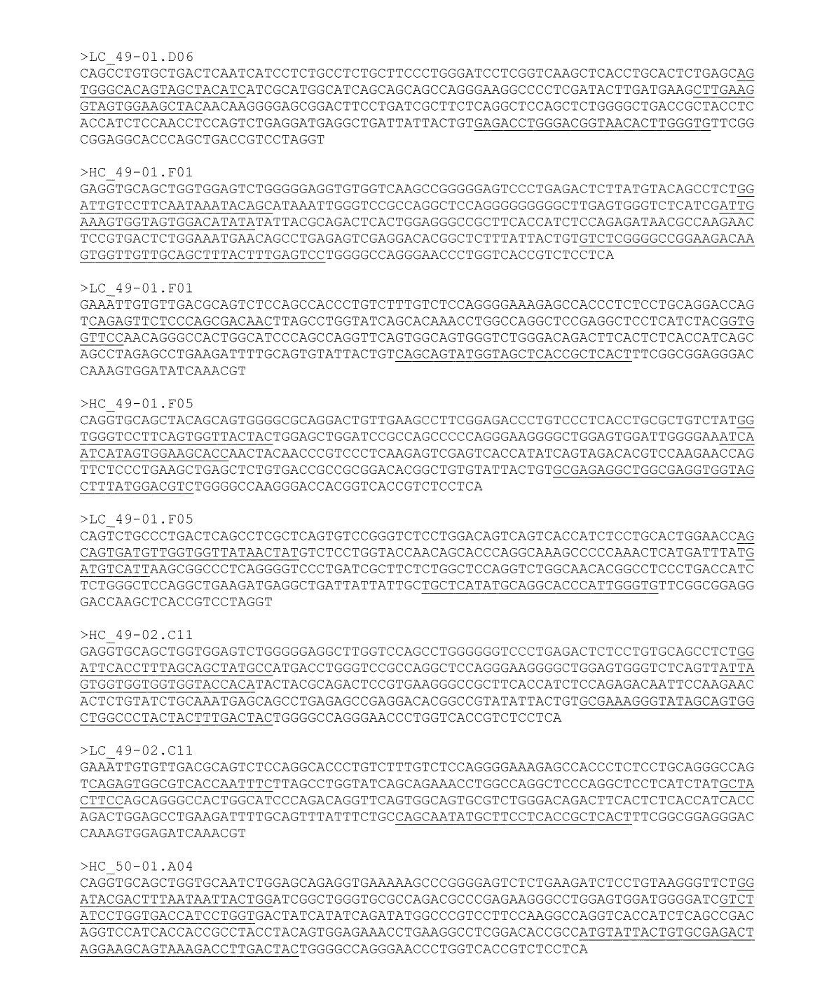

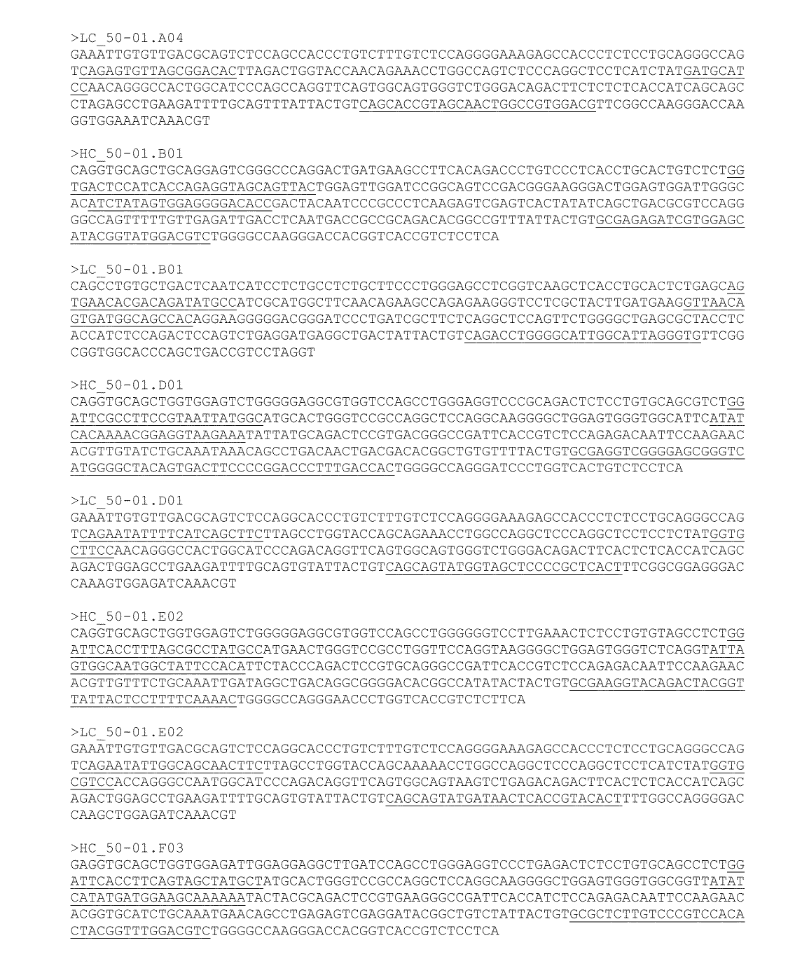

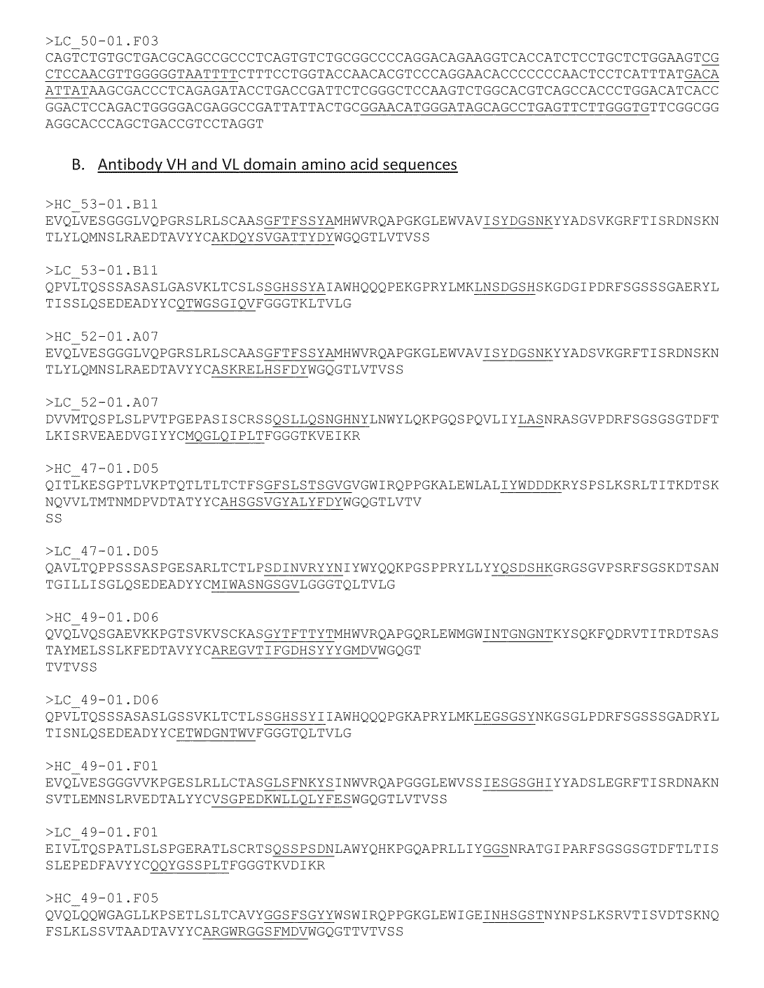

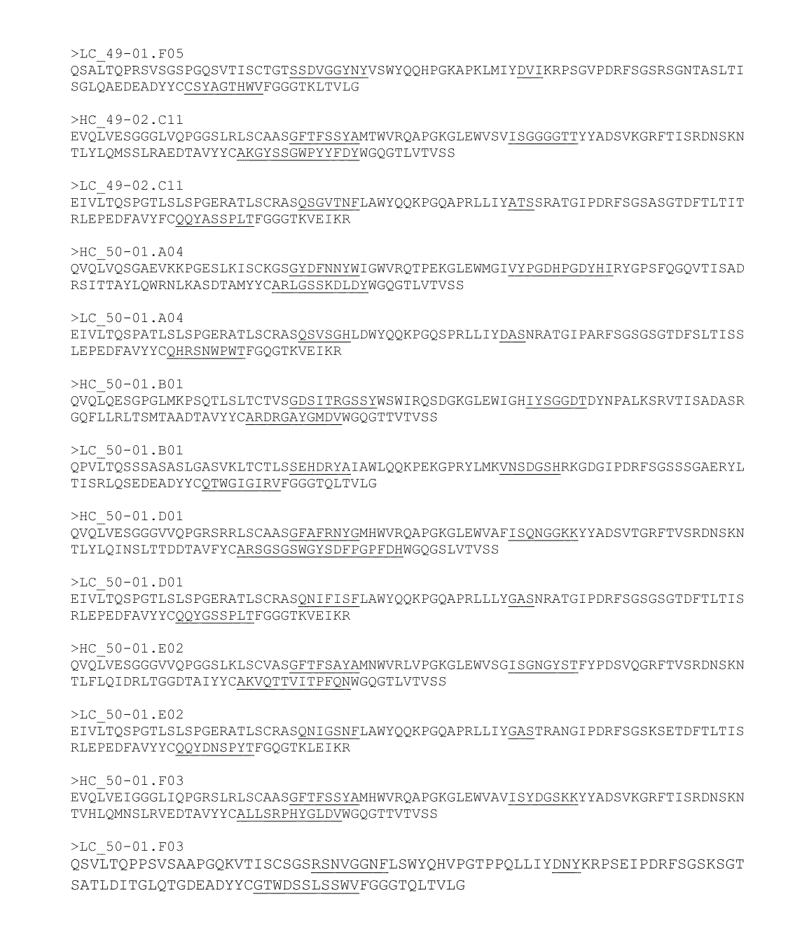

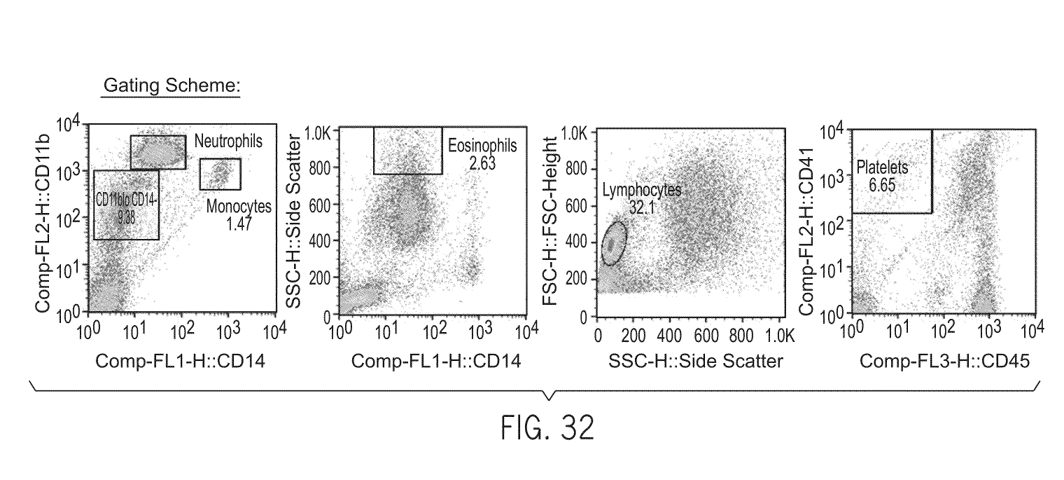

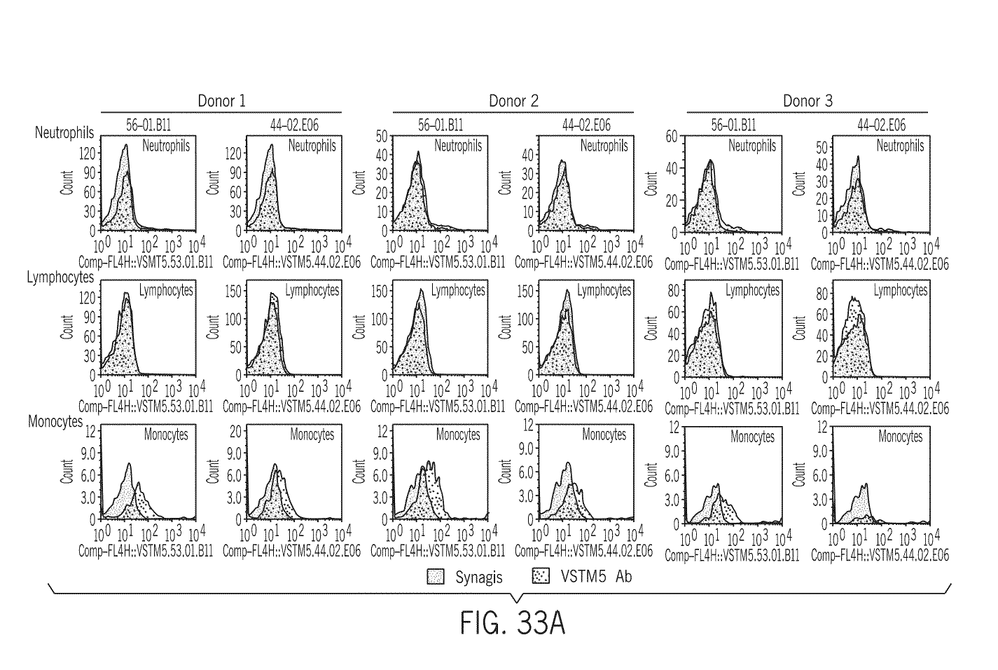

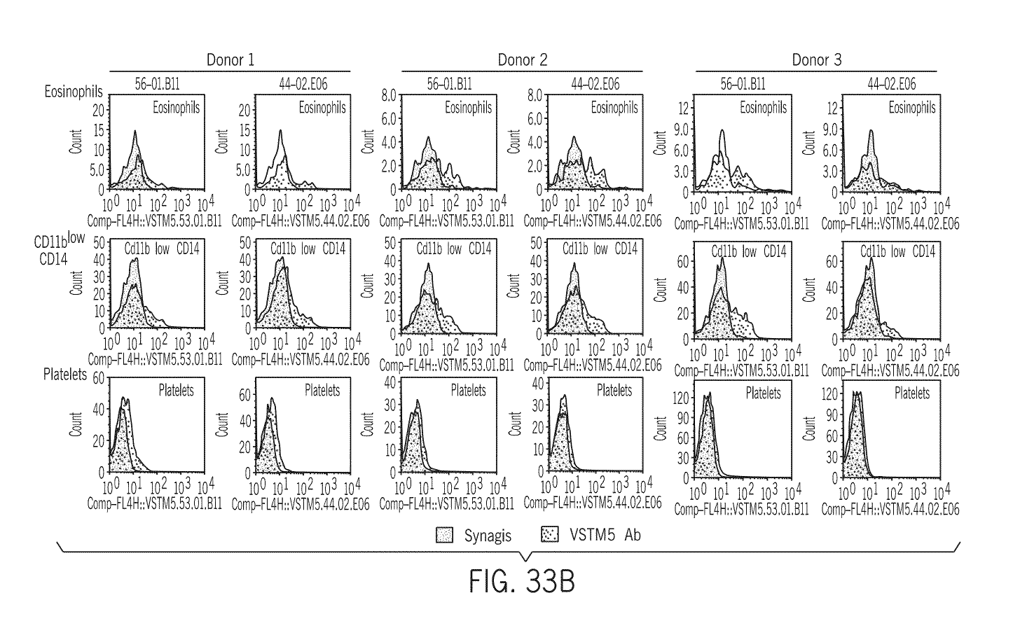

The gating scheme for the FACS assay is contained in FIG. 32. The binding results of the FACS assay for different cell types are shown in FIG. 33. As shown by the data in FIG. 33 VSTM5 is highly expressed by monocytes, CD1b1^{low}CD14⁻ cells, and to a lesser degree by eosinophils obtained from 3 healthy donors. No expression was observed on lymphocytes, neutrophils, and platelets. This data demonstrates that VSTM5 is expressed by many different types of human immune cells, in particular monocytes.

### Example 16

### In-Vitro Functional Testing of Anti-VSTM5 Antibodies in Co-Culture Assays

In order to evaluate the effect of the native cell surface expressed VSTM5 protein on T cell activation, a co-culture assay of HEK-293T cells over-expressing VSTM5 and H9 (clonal derivative of the Hut 78 cell line derived from a human T cell leukemia) activated by plate-bound anti-CD3 antibodies were used. The Materials & Methods used in these experiments are described below.

Materials & Methods

Anti-CD3 Mediated Activation of H9 Cells as Measured by Human IL-2 Cytokine Secretion in the Supernatant

The conditions for anti-CD3 Mediated Activation of H9 Cells and detecting activation based on IL-2 secretion was effected by the following experimental protocol.

Day 1:

HEK-293T cell pools stably transfected with expression constructs of the pRp3.1 plasmid expressing VSTM5, or with the empty vector pRp3.1, were seeded at a concentration of 7×106 cells per T75 plate and cultured in DMEM medium supplemented with 10% FBS, L-glutamine in a humidified incubator O.N.

Day 2:

Anti-CD3 (Clone OKT3, eBioscience; cat#16-0037) diluted in 1×PBS was immobilized on a flat-bottom 96-well plate in 75 μL/well at a concentration of 0.1 and 0.2 μg/ml.

Plates were wrapped with parafilm and incubated at 37° C. for 3 hours in humidified incubator.

Wells coated with anti-CD3 were washed 3 times with 200 μl of PBS. Fluid was decanted in a sterile environment. After the last wash, the plate was blotted on a sterile absorbent paper to remove any residual fluid.

HEK-293T cells, seeded the day before, were treated with mitomycin C (Sigma, cat# M4287): 900 μl of a 0.5 mg/ml solution freshly prepared in H₂O were added directly to 8.1 ml of growth medium, to obtain a final concentration of 50 μg/ml. Cells were incubated with mitomycin C for 1 hour at 37° C.

Mitomycin C treated HEK 293T cells were washed 3 times with 10 ml of PBS and detached by addition of 2 ml of cell dissociation buffer (Gibco; cat#13151-014).

Detached HEK-293T cells were re-suspended in 8 ml of RPMI supplemented with 10% FBS and L-glutamine (H9 cell growth medium).

Cells were counted using a Beckman coulter counter and diluted to 0.5×10⁶ cells per ml.

Cells were serially diluted and seeded at the indicated concentrations (50,000 and 75,000 HEK-293T) in 100 μl per well of H9 cells' growth medium (described above).

HEK-293T cells were incubated for 2 hours to allow attachment. 50,000 of the H9 cells (ATCC, HTB-176) were added to each well at a volume of 100 μl per well in H9 cells' growth medium (described above).

Cells were co-cultured O.N. at 37° C. in a humidified incubator.

Day 3:

Cells were transferred to U-shape plates, centrifuged 5 minutes at 1500 rpm at 4° C. The supernatant was frozen and kept at −20° C. until an interleukin 2 (IL-2) immunoassay was performed. The additional early marker of T cell activation (CD69) was tested in the same experiment but not selected for use as a marker as it showed inconclusive results.

Quantitative Determination of Human Interleukin 2 (IL-2) Concentrations in Cell Culture Supernates

In order to assess the response of the T cells, cytokine secretion (hIL-2) was measured by ELISA in culture supernatants, diluted to be in the linear range of the ELISA assay (R&D Systems, Quantikine ELISA, Human IL-2, cat# S2050) using the following experimental protocol.

All the particles (cells and cell debris) were removed by centrifugation and the supernatants were sealed and kept at −20° C.

The reagents, samples and working standards are prepared as directed in assay procedure. The samples are diluted 5 times in PBS.

Assay Diluent RD1W is added (100 μl) to each well.

The standard (duplicates) and the samples (triplicates) are added to the wells, covered with adhesive strip and incubated 2 hours at room temperature.

Each well was aspirated and washed (Wash buffer supplied within kit) 3 times and traces of the wash buffer were removed by blotting the plate against clean paper.

II-2 conjugate is added to each well, covered with adhesive strip and incubated 2 hours at room temperature.

Aspiration/wash step (5) is repeated.

The substrate solution (provided by the kit) is added to each well and incubated 20 minutes at room temperature.

The reaction is stopped by stop solution (provided by the kit).

The optical density of each well is determined by using a microplate reader (Biotek, ELx808) set to 450 nm.

Results

Inhibition of Anti CD3-Mediated Activation of H9 T Cells as Measured by Cytokine (IL-2) Secretion.

HEK-293T transfectants expressing the full length of human VSTM5 protein were co-cultured with H9 T cells activated by plate-bound anti-CD3 antibodies, as described in the Material & Methods supra. HEK-293T cells transfected with vector only (pRp3.1) (lacking VSTM5 sequence) were used as a negative control.

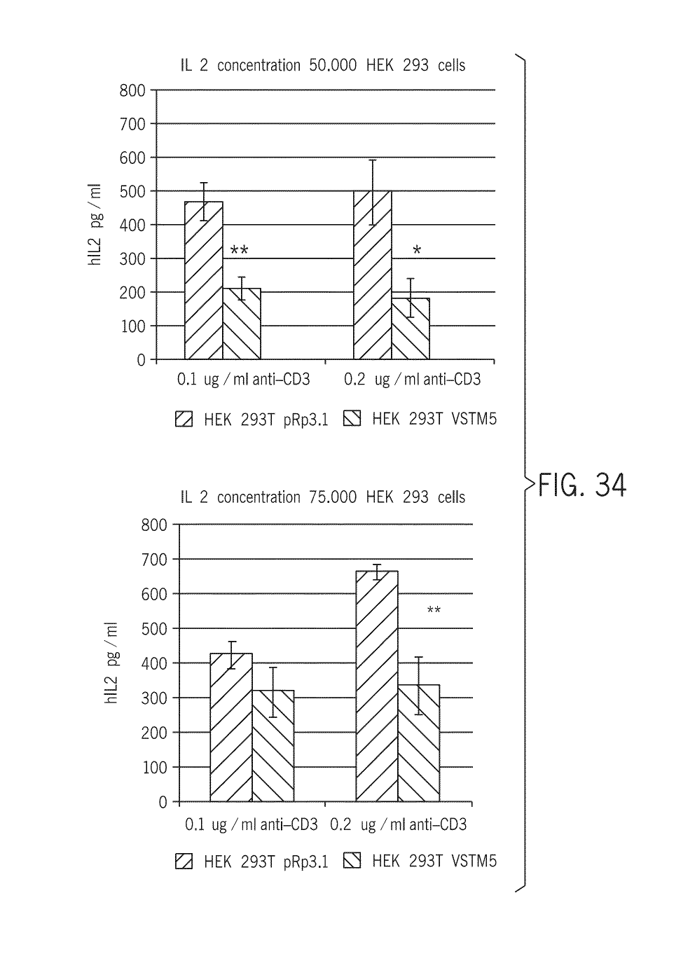

Representative results are shown in FIG. 34. These results indicate that H9 T cells stimulated with anti-human CD3 antibody exhibit reduced activation in the presence of VSTM5-expressing HEK-293T cells, as evidenced by the reduced secretion of IL-2 in the supernatant, compared to the control HEK-293T cells which were transfected with the vector only (pRp3.1). As shown in FIG. 34 the inhibitory effect of VSTM5 was the most prominent in the experiments using 50,000 HEK293T transfected cells per well.

These results show that VSTM5 expressed on the cell membrane of HEK-293T cells inhibits H9 T cell activation, and further indicate that the native VSTM5 membrane protein expressed on the cell surface inhibits T cell activation. This data further corroborates the immunosuppressive effect of VSTM5 on T cell activation and suggests that binding agents which agonize or antagonize VSTM5 may be used to modulate T cell activity and treat conditions such as discussed herein wherein enhanced or decreased T cell activity will be therapeutically beneficial.

### Example 17

### Decreased Inhibition of Anti-CD3-Mediated Activation of H9 T Cells by Anti-VSTM5 Abs as Measured by IL-2 Cytokine Secretion

Based on the results of the co-culture experiments in Example 15, the functional effect of VSTM5 binding agents, i.e., anti-VSTM5 specific Abs on T cell activation in the presence of VSTM5 expressing T cells was tested in the same co-culture assay, except that these assays were performed in the presence of different hIgG1 anti-VSTM5 Abs¹. ¹ The methods used to derive the anti-VSTM5 antibodies referenced in this example are described in Example 12.

In these experiments HEK 293T cells expressing VSTM5 protein were co-cultured with H9 T cells activated by plate-bound anti-CD3 antibodies in the presence or absence of said hIgG1 anti-VSTM5 Abs. The anti-VSTM5 Abs were added to a final concentration of 20 ug/ml in a total volume of 50 ul of H9 cell growth medium 2 hours prior to the addition of the H9 cells. HEK 293T cells transfected with the vector only (pRp3.1) were again used as a negative control.

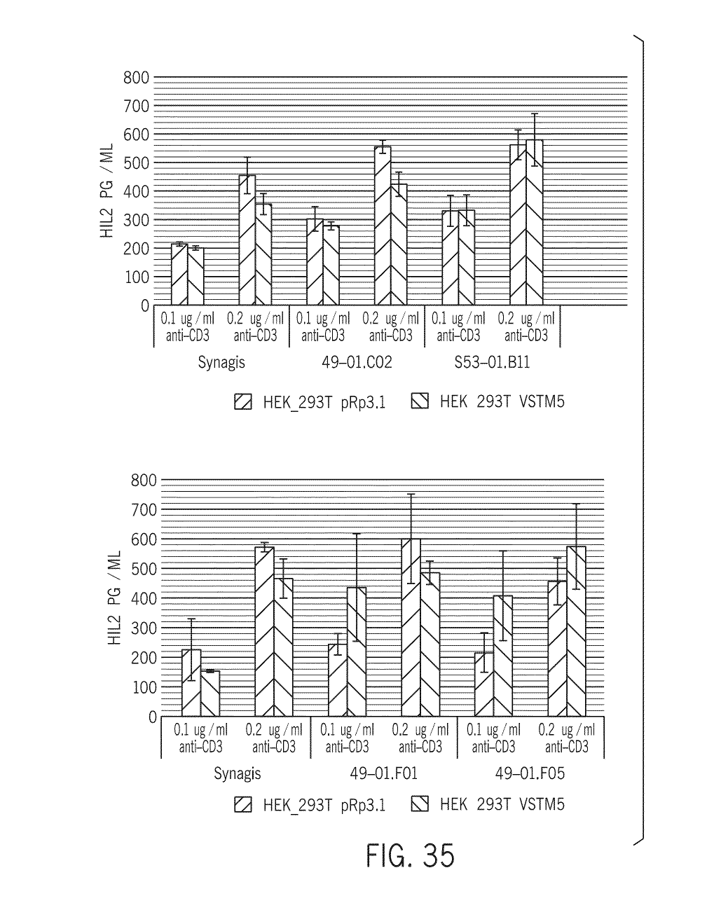

The results of these initial experiments shown in FIG. 35 indicate that addition of at least 2 VSTM5 specific hIgG1 Ab (49-01.F05 and S53-01.B11) increase the activation H9 cells, thus reducing the inhibitory effect mediated by the cell surface expressed VSTM5 protein when compared to control Abs (Synagis and 49-01.C02/the same production non-binder). By contrast, another tested anti-VSTM5 antibody (49-01.F01) under these same conditions did not antagonize the suppressive effects of VSTM5 on T cell activity (as evidenced by it eliciting no modulatory effect on IL-2 secretion).

This observation suggests that some Ab's which specifically bind to cell surface expressed VSTM5 potentially may be used to inhibit or neutralize the inhibition of T cell activation and activity mediated by VSTM5.

As shown in FIG. 35 in this co-culture assay a number of anti-VSTM5 antibodies were demonstrated to reduce the inhibitory effect of VSTM5 (expressed by HEK-293T cells) on H9 activated cells as measured by IL-2 secretion. Particularly, in these co-culture assays HEK 293T cells expressing VSTM5 or the empty vector (pRp3.1) were seeded in wells pre-coated with 0, 0.1 and 0.2 μg/ml of anti-CD3 antibody. VSTM5 specific Abs (S53-01.B11 and 49-01.F05) represented in (A) and (B) respectively and control Abs (Synagis, non-binding Abs 49-01.C02) were added to a final concentration of 20 ug/ml 2 hours prior to addition of H9 cells and the co-cultures were incubated O.N. Supernatants, depleted from the cells, were analyzed for concentration of hIL-2 Standard deviation of triplicates are shown in FIG. 35.

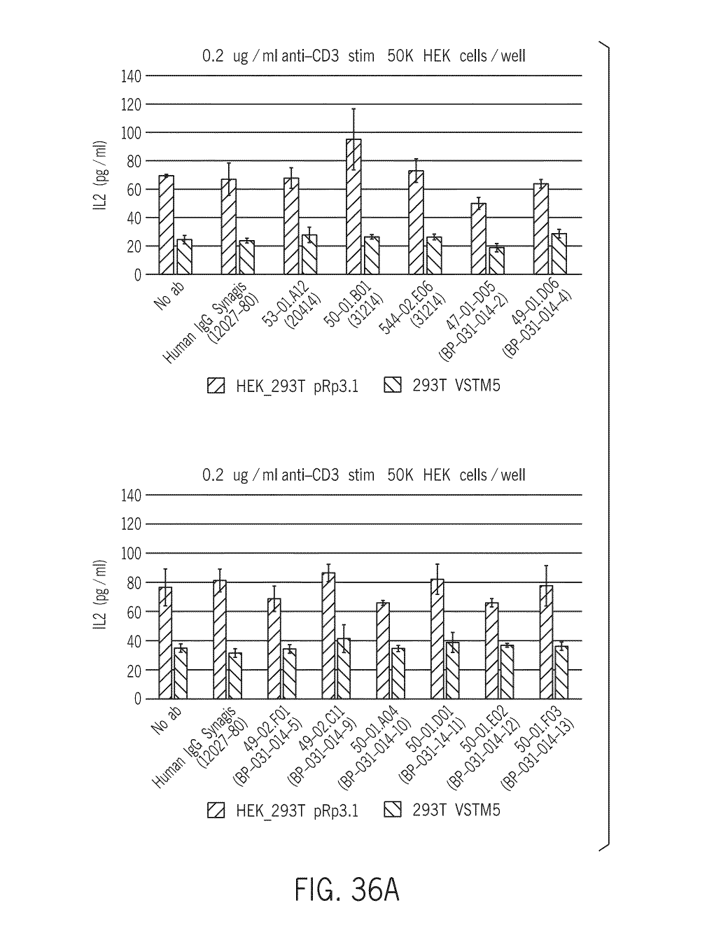

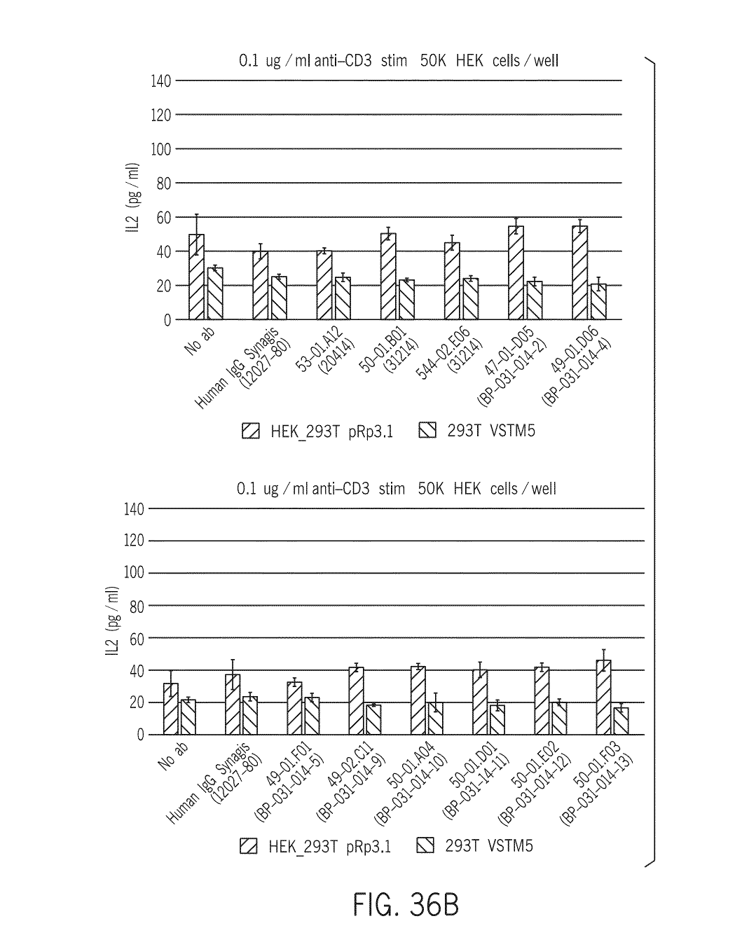

In an additional experiment shown in FIG. 36, other anti-VSTM5 antibodies were tested for their ability to modulate the suppressive effect of VSTM5 on T cell activity using the same co-culture assay described above. Specifically, in these experiments the effect of specific antibodies produced against VSTM5 to inhibit the effects of VSTM5 (expressed on the surface of HEK-293T cells) on H9 activated cells was again assayed based on their effect on IL-2 secretion. In these experiments HEK 293T cells expressing VSTM5 or the empty vector (pRp3.1) were again seeded in wells pre-coated with 0.1 and 0.2 μg/ml of anti-CD3 antibody. The anti-VSTM5 Abs and control Abs (Synagis) were added to a final concentration of 20 ug/ml 2 hours prior to the addition of H9 cells and the co-cultures were incubated O.N.

Supernatants, depleted from the cells, were then analyzed for concentration of hIL-2 Standard deviation of triplicates are indicated in FIG. 36. As shown by the data in FIG. 36, this panel of anti-VSTM5 antibodies when tested in this co-culture assay, did not elicit detectable increases in IL-2 when these anti-VSTM5 antibodies were added to HEK293T (expressing VSTM5)/H9 cell co-cultures.

While Applicants do not want to be bound by this hypothesis, it is theorized that some of the anti-VSTM5 antibodies tested in this experiment did not modulate the suppressive effect of VSTM5 on T cell activity as they may bind VSTM5 at an epitope that is not involved in VSTM5 activity or its interaction with its counter receptor (i.e., non-functional binding which could explain the absence of any detectable effect on VSTM5 mediated suppression of T cell activity). Alternatively, these antibodies may bind VSTM5 at an epitope which is involved in eliciting other immunosuppressive effects of VSTM5 on immunity, i.e., other than IL-2 production.

### Example 18

### Restoration of T Cell Activation by Anti-VSTM5 mAbs in VSTM5-ECD-Ig Fusion Coated Bead Assay

The experiments described in this example assess the ability of different anti-VSTM5 mAbs in blocking the inhibitory effect of VSTM5-ECD-Ig fusion protein on T cell activity in an analogous bead assay. As described below and shown in the Figures referenced herein, using these bead assay conditions several anti-VSTM5 mAbs were identified which inhibited or neutralized the inhibitory effect of VSTM5-ECD-Ig on T cell activity and which were shown to restore the activation of T cells. The Materials & Methods used in these experiments are described below.

Materials and Methods

Bead Coating and QC:

Tosyl activated beads (Invitrogen, Cat#14013) at 500×10⁶/ml were coated with anti-CD3 mAb and Fc fusion proteins in a two-step protocol: with 50 ug/ml human anti-CD3 clone UTCH1 (R&D systems, Cat# mab 100) in sodium phosphate buffer at 37° C. overnight, followed with VSTM5-ECD-Ig fusion (human ECD of VSTM5 fused with human IgG1) for another overnight incubation at 37° C. In the second step, control human Fc (Bioxcell, Cat# BE0096) was added together with Fc fusion protein so that the total amount of protein was 160 ug/ml.

VSTM5-ECD-Ig fusion levels on the beads were assessed using Alexa 647 conjugated anti-VSTM5 mab 53-01.B11 (Lot 20414), and PD-L1 Fc levels by anti-PD-L1 (ebioscience, Cat#14-9971-81) followed by goat-anti-mouse 647 (1:200) (Jackson Immuno Research, Cat#115-606-146). Anti-CD3 levels on beads were assessed using goat anti-mouse 647 (Jackson ImmunoResearch, Cat#115-606-146)

Bead Assay Setup:

100 k human CD3⁺ T cells were cultured with 100 k or 200 k coated beads in the presence of 10 ug/ml of anti-VSTM5 mAb, anti-PD-L1 mAb, or hIgG1 control Synagis for 5 days in complete IMDM (Gibco, Cat #12440-053) supplemented with 2% AB human serum (Gibco, Cat#34005-100), Glutmax (Gibco, Cat #35050-061), sodium pyruvate (Gibco, Cat #11360-070), MEM Non-Essential Amino Acids Solution (Gibco, Cat #11140-050), and 2-mercaptoethanol (Gibco, Cat #21985). At the end of 5 day culture, cells were stained with anti-CD25, anti-CD4, anti-CD8, and fixable live dead dye to determine CD25 expression levels on each subset of cells. Medium fluorescence intensity (MFI) value of CD25 was normalized against Synagis control condition for each bead type (VSTM5-ECD-Ig fusion coated beads and control human IgG1 Fc coated beads). Supernatants were collected and assayed for IFNγ secretion by ELISA (Human INFγ duoset, R&D systems, DY285).

Results

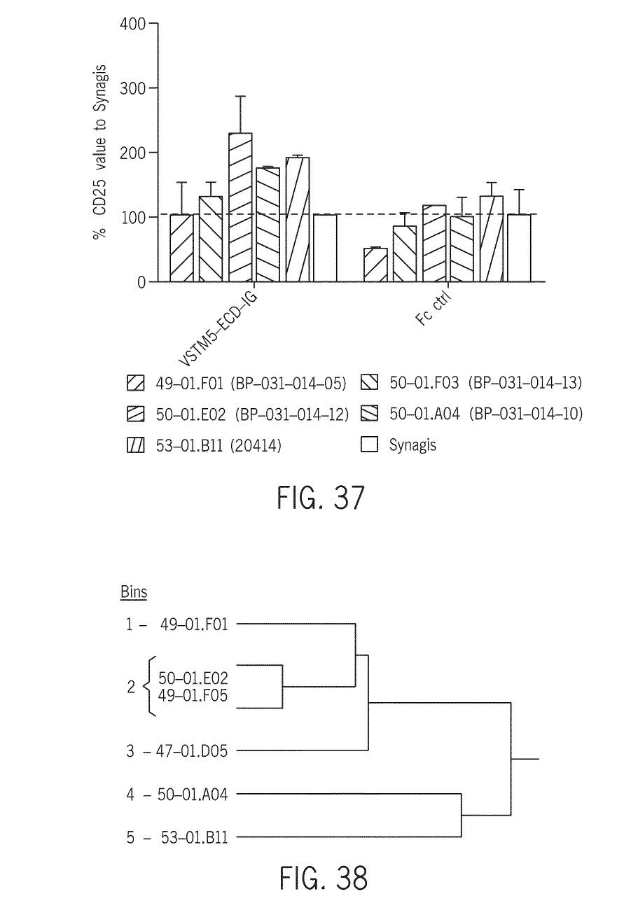

Nine mAbs against VSTM5 were tested in the above-described bead assay using two cell:bead ratios. Activation of CD4⁺ and CD8⁺ cells were assessed by CD25 expression and IFNγ secretion as described in the Materials and Methods supra. As shown in FIG. 37, three mAbs (50-01.E02, 50-01.A04, 53-01.B11) substantially increased CD25 expression on CD4⁺ T cells, when tested at a ratio of cell:bead of 1:2; and 1 mAb (50-01.F03) elicited a marginally positive effect. Five mAbs (49-01.F01, 49-01.D06, 47-01.D05, 49-01.F05, 49-02.C11) did not show an enhancing effect specific to VSTM5 bead conditions. Also, no differential enhancing effect on CD25 expression was observed for any of the tested anti-VSTM5 Abs when tested at cell: bead ratio of 1:1 and the levels of detected IFNγ secretion fluctuated under the tested cell: bead ratios.

Review

The data in FIG. 37 indicate that three of the nine tested mAbs against VSTM5 elicited neutralizing activities against VSTM5-ECD-Ig proteins and restored T cell activation in the above-described bead assays. Based thereon, these bead assays may be used to select other anti-VSTM5 Abs which modulate the immunosuppressive effect of VSTM5, e.g., on T cell activation and potentially the secretion of proinflammatory cytokines such as IFNγ.

### Example 19

### Surface Plasmon Resonance Study of Epitope Binning Anti-VSTM5 IgG Antibodies Binding to Monomeric VSTM5 Antigen

In the experiments described herein surface plasmon resonance binding assays were used to bin eight unique anti-VSTM5 monoclonal antibodies based on pair-wise antigen epitope blocking between all eight mAbs. The Materials & Methods used are described below.

Materials and Methods

Epitope binning experiments were performed using a ProteOn XPR 36 instrument at 22° C.

Step 1:

The following anti-VSTM5 mAbs were each diluted to ˜12 μg/mL in 10 mM sodium acetate, pH 4.5 and covalently immobilized to a ProteOn GLC biosensor chip using standard amine coupling:

| | |
|---|---|
| 49-01.F01 (lot#BP-031-014-5) | 50-01.F03 (lot#BP-031-014-13) |
| 50-01.E02 (lot# BP-031-014-12) | 50-01.A04 (lot#BP-031-014-10) |
| 53-01.B11 (lot#20414) | 47-01.D05 (lot# BP-031-014-2) |
| 49-01.F05 (lot# 3101414) | 50-01.B01 (lot# n/a) |

The activation step occurred in the horizontal flow direction while the immobilization step occurred in the vertical flow direction. The blocking step occurred in both the vertical and horizontal positions so that the horizontal “interspots” could be used as reference surfaces. Each mAb was immobilized at a range of ˜4300 RU-4800 RU.

Step 2:

Preliminary experiments showed relatively fast dissociation times of the VSTM5 monomer from most of the mAbs listed in Step 1. Therefore a “pre-mix” binning protocol was performed where each mAb listed in Step 1 was pre-mixed with VSTM5 monomer with the molar binding site concentration of each mAb in excess of the molar antigen concentration. The final binding site concentration of each mAb was ˜500 nM and the final monomer concentration was 25 nM.

Step 3:

Each mAb/VSTM5 sample was injected over all covalently immobilized mAbs. Control injections included 1) each mAb injected without antigen at concentrations identical to the pre-mix samples from Step 2, 2) several injections of antigen at 25 nM without mAb, and 3) buffer injections for double-referencing. All pre-mix samples and control samples were injected for 2 minutes followed by 5 minutes of dissociation at a flow rate of 50 μL/min. Surfaces were regenerated with a 30 sec pulse of 10 mM glycine-HCl, pH 2.5. Running buffer was PBS with 0.005% Tween 20 and 100 μg/mL BSA.

Step 4:

Sensorgram data were processed and double-referenced using ProteOn Manager Version 3.1.0.6. An antibody pair was classified as having a shared antigen binding epitope if no binding was observed from the injection of mixed mAb and antigen over the immobilized mAb, or if binding was significantly reduced as compared to the antigen-only control injection over the same immobilized mAb. Conversely, an antibody pair was classified as binding to different antigen epitopes if the injection of mixed mAb and antigen showed binding to the immobilized mAb similar to the antigen-only control over the same immobilized mAb. (In these experiments, 2 anti-VSTM5 antibodies, 50-01.F03 and 50-01.B01 were removed from binning considerations because of their relatively low binding affinities to VSTM5 antigen.)

Step 5

A binary matrix of all mAb pairs was constructed with “0” given to mAb pairs that appeared to share antigen binding epitopes and “1” given to mAb pairs that appeared to have different binding epitopes. Hierarchical clustering of the matrix was performed using JMP software and a clustering dendrogram was generated to identify the epitope bins.

Results

The resulting dendrogram indicating five epitope bins for the six remaining anti-VSTM5 mAbs is shown in FIG. 38. Table 4 below lists the mAbs, their equilibrium binding constants (where applicable), bead and co-culture assay results, and their respective epitope bins.

TABLE 1
Table 4 mAB

| | | | VSTM5 | | |
|---|---|---|---|---|---|
| | | Co- | Monomer | | Mouse |
| | Bead | Culture | Binding | Epitope | VSTM5 |
| | Assay | Assay | (KD) | Bin | Binding |
| 49-01.F01 | Neg. | Neg. | 2 × 10⁻⁹ M | 1 | No |
| 50-01.F03 | Border- | Neg. | Unable to | Unable to | No |
| | line | | Determine | Determine | |
| | | | (complex | (weak binder) | |
| | | | kinetics) | | |
| 50-01.E02 | Pos. | Neg. | Unable to | 2 | No |
| | | | Determine | | |
| | | | (complex | | |
| | | | kinetics) | | |
| 50-01.A04 | Pos. | Neg. | 5.28 × 10⁻⁸ M | 4 | No |
| 53-01.B11 | Pos. | Pos. | Unable to | 5 | Yes |
| | | | Determine | | |
| | | | (complex | | |
| | | | kinetics) | | |
| 47-01.D05 | Neg. | Neg. | 6.07 × 10⁻⁸ M | 3 | No |
| 49-01.F05 | Neg. | Pos. | 1.90 × 10⁻⁷ M | 2 | No |
| 50-01.B01 | Neg. | Neg. | 6.57 × 10⁻⁷ M | Unable to | Yes |
| | | | | Determine | |
| | | | | (weak binder) | |

### Example 20

### Role of VSTM5 Proteins as Modulators of Cancer Immune Surveillance: In Vivo

Mouse Cancer Syngeneic Models:

Transplantation of Tumor Cells Over-Expressing VSTM5 Proteins or a Non-Relevant Control Protein into Genetically Matched Mice.

In these experiments tumor cells over-expressing VSTM5 proteins or a non-relevant control protein were introduced into genetically matched mice. Tumor volume (and tumor weight after sacrificing the animals) are then examined to demonstrate delay in the tumor growth (i.e. tumor over expressing VSTM5 grow faster than tumors over expressing the non-relevant control protein). Also, ex vivo analysis of immune cells from tumor draining lymph nodes is carried out to evaluate the ratio of regulatory T cells and effector T cells.

As VSTM5 has been shown in the Examples above to inhibit the activation and proliferation of effector T cells in vitro and to promote the induction of iTRegs, these assays should demonstrate that the tumor samples from mice with tumors overexpressing VSTM5 contain a reduced number of effector T cells and a greater number of regulatory T cells than mice with tumors not overexpressing VSTM5 because of the immunosuppressant effect of VSTM5.

Treatment of Mice with a Syngeneic Tumor with Immunostimulatory Antibodies Directed Against VSTM5 Protein as Mono-Therapy or with an Irrelevant Isotype-Matched Antibody.

Mice with a syngeneic tumor are treated with immunostimulatory antibodies directed against VSTM5 protein as mono-therapy or with an irrelevant isotype-matched antibody. In these experiments tumor cells transplanted to genetically identical mice. Tumor bearing mice are injected with different doses of antibodies against VSTM5 protein that have been shown to antagonize the immunosuppressant effects of VSTM5 on immunity.

Treatment with immunostimulatory antibodies specific for VSTM5 protein is anticipated to demonstrate that there is greater anti-tumor immunity against the tumor in mice treated with the immunostimulatory antibody against VSTM5 protein and that their tumors grow slower than tumors in mice treated with an irrelevant antibody of the same isotype. Also, ex vivo analysis of immune cells from tumor draining lymph nodes is similarly carried out to determine the ratio of regulatory T cells and effector T cells after treatment.

Again, as VSTM5 has been shown in the Examples above to inhibit the activation and proliferation of effector T cells in vitro and to promote the induction of iTRegs, these as ays should demonstrate that the tumor samples from mice with tumors overexpressing VSTM5 contain an increased number of effector T cells and a reduced number of regulatory T cells than mice treated with the irrelevant antibody because of the immunostimulatory effect of the anti-VSTM5 and its inhibitory effect on the immunosuppressive effects of VSTM5 on Effector T cells and its potentiating effect on TREGs.

Testing of Tumor Cells Lines are Tested from Various Sources Including Colon, Breast, and Ovary Carcinomas, Melanoma, Sarcomas and Hematological Cancers.

Tumor cells lines are tested from various sources including colon, breast, and ovary carcinomas, melanoma, sarcomas and hematological cancers. Using these cells syngeneic models are performed in several mouse strains including BALB/c, C57bl/6 and C3H/Hej. In the first set of experiments the syngeneic transplantable models used are primarily those which have been established to be reliably predictive for cancer immunotherapy. These include: B16-F10 melanoma (according to the method described in Tihui Fu et al Cancer Res 2011; 71: 5445-5454), MC38 colon cancer (according to the method described in Ngiow S F et al. Cancer Res. 2011 May 15; 71(10):3540-51), ID8 ovarian cancer (according to the method described in Krempski et al. J Immunol 2011; 186:6905-6913), MCA105 sarcoma (according to the method described in Wang et al. J. Exp. Med. Vol. 208 No. 3 577-592), CT26 colon carcinoma (according to the method described in Ngiow S F et al. Cancer Res. 2011 May 15; 71(10):3540-51) and 4T1 mammary carcinoma (according to the method described in Takeda K et al. J Immunol. 2010 May 15; 184(10):5493-501) of BALB/c background.

Establishment of a Syngeneic Tumor and Treatment with Immunostimulatory Antibodies Directed Against VSTM5 Protein in Combination with Additional Lines of Treatment.

Tumor cells are transplanted to genetically identical mice. After the establishment of tumors, mice are injected IP with different doses of immunostimulatory antibodies aimed against VSTM5 protein in combination with conventional chemotherapy (e.g. cyclophosphamide, according to the method described in Mkrtichyan et al. Eur. J Immunol. 2011; 41, 2977-2986), in combination with other immune checkpoint blockers (e.g. PD1 and CTLA4, according to the method described in Curran et al.; Proc Natl. Acad Sci USA. 2010 Mar. 2; 107(9):4275-80), in combination with other immune-modulators (e.g. anti-IL-18, according to the method described in Terme et al.; Cancer Res. 2011; 71: 5393-5399), in combination with cancer vaccine (according to the method described in Hurwitz et al. Cancer Research 60, 2444-2448, May 1, 2000) or in combination with radiotherapy (according to the method described in Verbrugge et al. Cancer Res 2012; 72:3163-3174).

It is anticipated that the immunostimulatory antibodies against VSTM5 will potentiate the antitumor effects of the chemotherapeutic, immune-modulators or other immune checkpoint blockers, and potentiate the antitumor efficacy of cancer vaccines as the suppression of the immunosuppressive effects of VSTM5 should promote antitumor immunity.

Human Cancer Xenograft Model:

Human cancer cell lines, endogenously expressing VSTM5 are transplanted into immune-deficient mice. Tumor volume in mice treated with anti-VSTM5 antibody is compared with mice treated with non-relevant isotype matched antibody. In one arm of the study anti-VSTM5 antibodies are conjugated to a toxin (according to the method described in Luther N et al. Mol Cancer Ther. 2010 April; 9(4):1039-46) to assess antibody drug conjugate (ADC) activity. In another arm of the experiment, mice are treated with human IgG1 or mouse IgG2a isotype antibodies against VSTM5 (according to the method described in Holbrook E. Kohrt et al. J Clin Invest. 2012 Mar. 1; 122(3): 1066-1075). These antibody isotypes are used to assess antibody-dependent cellular cytotoxicity (ADCC) mediated tumor elimination.

Expression of VSTM5 Proteins on Tumor and Immune Cells Isolated from Human Tumor Biopsies

Expression validation of VSTM5 proteins using specific antibodies directed against the VSTM5 proteins is carried out on separated cell populations from the tumor. Various cell populations are freshly isolated from tumor biopsies (e.g. Tumor cells, endothelia, tumor associated macrophages (TAMs) and DCs, B cells and different T cell sub-sets (CD4, CD8 and Tregs) as described in Kryczek I. et al., J. Exp. Med; 2006; Vol. 203; p. 871-881 and Cancer Res. 2007; 67; 8900-8905, to demonstrate expression of VSTM5 in tumor cells and on tumor stroma and immune infiltrate.

A binding assay is then performed with human VSTM5-ECD-Ig proteins on separated cell populations from the tumor. Various cell populations from tumor biopsies (e.g. Tumor cells, endothelia, tumor associated macrophages (TAMs) and DCs, B cells and different T cells (CD4, CD8 and Tregs) are freshly isolated from tumors as described in J. Exp. Med.; 2006; Vol. 203; p. 871-881 and Cancer Res. 2007; 67; 8900-8905, to show expression of the counter receptor for VSTM5 in tumor cells and on tumor stroma and immune cells.

Based on the studies in Example 1, and the other above examples, it is anticipated that many of these tumor, stromal and immune cells will express VSTM5 and that increased VSTM5 expression will correlate to reduced antitumor activity by the subject's immune cells.

Expression of VSTM5 Proteins on Cells Isolated from Draining Lymph Nodes and Spleens of Tumor Bearing Mice

The expression of VSTM5 proteins by immune cells of tumor bearing mice is further assayed using specific antibodies directed against VSTM5 proteins and is effected using epithelial cancer cells as well as on immune cells from tumor draining lymph nodes and compared to spleen samples of tumor bearing C57 mice, as described in M Rocha et al., Clinical Cancer Research 1996 Vol. 2, 811-820. Three different cancer types are tested: B16 (melanoma), ID8 (ovarian) and MC38 (colon)), in order to evaluate expression of VSTM5 in tumor cells and in immune cells within the tumor draining lymph node.

A binding assay with mouse VSTM5-ECD-Ig proteins on cells isolated from epithelial cancer as well as on immune cells from tumor draining lymph nodes compared to spleen of tumor bearing C57 mice is also effected as described above, to establish the expression of the counter receptor for VSTM5 in tumor cells and in immune cells including NK cells in the tumor draining lymph node.

Based on the studies in Example 1, and the afore-examples, it is anticipated that many of these immune cells will express VSTM5 and that increased VSTM5 expression will correlate to reduced antitumor anti-immunity.

Expression of VSTM5 Proteins on M2 Polarized Macrophages

The expression of VSTM5 proteins is further assayed using specific antibodies directed against VSTM5 proteins, against primary monocytes isolated from peripheral blood, differentiated into macrophages and exposed to “M2 driving stimuli” (e.g. IL4, IL10, Glucocorticoids, TGF-β), as described in Biswas S K, Nat. Immunol. 2010; Vol. 11; p. 889-896, to show expression of VSTM5 in M2 differentiated Macrophages. It is anticipated that the assay will validate the expression of VSTM5 by these cells.

Further another binding assay was conducted using VSTM5 human ECD-FC proteins and primary monocytes isolated from peripheral blood, differentiated into macrophages and exposed to “M2 driving stimuli” (e.g. IL4, IL10, glucocorticoids, TGF-β) is carried out as described above, to evaluate expression of the counter receptor for VSTM5 in M2 differentiated macrophages. Again, it is anticipated that the assay will confirm the expression of VSTM5 by these cells.

Expression of VSTM5 Proteins on Myeloid Derived Suppressor Cells (MDSCs)

Another experiment is conducted which further assays the expression of VSTM5 proteins using specific antibodies directed against VSTM5 proteins, respectively, on primary MDSCs isolated from Tumor bearing mice, as described in Int. Immunopharmacol. 2009 July; 9(7-8):937-48. Epub 2009 Apr. 9. It is anticipated that the assay will confirm the expression of VSTM5 by these cells based on the established immunosuppressive effect of VSTM5 and its potentiating effect on suppressor cells.

Binding assays are carried out with VSTM5 human ECD-Fc proteins (as described in PCT/IB2012/051868, incorporated by reference herein) and owned in common with the present application, on primary MDSCs isolated from tumor bearing mice. It is anticipated that the assay will confirm the expression of VSTM5 by these cells based on the established immunosuppressive effect of VSTM5 and its potentiating effect on suppressor cells.

### Example 21

### Anti-Tumor Effect of Immunostimulatory Antibody Against the VSTM5 Protein in Combination with Blockade of Known Immune Checkpoints

Inhibitory receptors on immune cells are pivotal regulators of immune escape in cancer. Among these are known immune checkpoints such as CTLA4, PD-1 and LAG-3. Blockade of a single immune checkpoint often leads to enhanced effector T cell infiltration of tumors, but may also lead to compensatory upregulation in these T cells of the other unblocked negative receptors. However, blockade of more than one inhibitory pathway allows T cells to carry out a more efficient tumor response, and increases the ratio of effector T cells (Teffs) to regulatory T cells (Tregs). Specifically, dual blockade of such inhibitory receptors has been shown to exert synergistic therapeutic effect in animal tumor models (Curran et al 2010 PNAS 107: 4275-4280; Woo et al 2011 Cancer Res. 72: 917-927). Based on these findings, the combination of anti-CTLA-4 and anti-PD-1 blocking antibodies is being tested in clinical trials in patients with metastatic melanoma.

The combination of blocking antibodies against VSTM5 and against PD-1 is tested in the syngeneic cancer MC38 model in the C57B1/6 background (as described in Woo et al 2011 Cancer Res. 72: 917-927). Briefly, MC38 cells (2×10⁶) are implanted s.c. C57B1/6 mice. Mice with palpable tumors are injected i.p. at a dosage of 10 mg/kg anti-VSTM5 mAb and/or anti-PD-1 mAb (4H2). Isotype Control Ab is dosed at 20 mg/kg or added to individual anti-PD-1 or anti-VSTM5 antibody treatments at 10 mg/kg. Tumor volumes are measured with an electronic caliper, and effect on tumor growth is calculated. The therapeutic effect, manifested as inhibition of tumor growth, is enhanced upon combination of the blocking antibodies against the two targets, PD-1 or VSTM5. The frequency of effector T cells=Teffs (CD8⁺ IFNγ⁺) cells and the ratio of Teffs and Tregs are determined in tumor draining lymph nodes and non-draining lymph nodes.

It is anticipated that antibodies which antagonize the immunosuppressive effects of VSTM5 will have at least an additive effect on T cell immunity when used in combination with other checkpoint blockers such as anti-PD-1 antibodies and may elicit as a synergistic benefit as these immune molecules may potentiate CTL cell activation and proliferation and NK mediated cytotoxicity via different immune pathways.

### Example 22

### Anti-Tumor Effect of Immunostimulatory Antibody Against VSTM5 Protein in Combination with Metronomic Therapy with Cyclophosphamide

Cyclophosphamide has been used as a standard alkylating chemotherapeutic agent against certain solid tumors and lymphomas because of its direct cytotoxic effect and its inhibitory activity against actively dividing cells. While high doses of cyclophosphamide may lead to depletion of immune cells, low doses have been shown to enhance immune responses and induce anti-tumor immune-mediated effects, primarily by reducing the number and function of immunosuppressive Treg cells (Brode and Cooke 2008 Crit. Rev. Immunol. 28: 109-126). Metronomic therapy using classical chemotherapies other than cyclophosphamide has also been shown to have immunostimulatory effects, including gemcitabine; platinum based compounds such as oxaliplatin, cisplatin and carboplatin; anthracyclines such as doxorubicin; taxanes such as paclitaxel and docetaxel; microtubule inhibitors such as vincristine.

Combination therapy of cyclophosphamide with other immunotherapies, such as anti-4-1BB activating Ab or anti-PD1 blocking Ab, resulted in synergistic anticancer effects (Kim et al. 2009 Mol Cancer Ther 8:469-478; Mkrtichyan et al. 2011 Eur. J. Immunol. 41:2977-2986).

Anti-VSTM5 blocking mAb is tested in combination with cyclophosphamide in the syngeneic B16 melanoma model in the C57BL/6 background (as described in Kim et al. 2009 Mol Cancer Ther 8:469-478). Briefly, C57BL/6 mice are injected s.c. with 4×105 B16-F10 melanoma cells. A single i.p. injection of cyclophosphamide (150 mg/kg) is administered on the day of tumor implantation, and five injections of 100 μg of the immunostimulatory antibody against VSTM5, 5 d apart beginning on the day of tumor implantation. To examine the antitumor effects of combination therapy on established tumors, the combination therapy is given beginning either at day 5 or day 10 after tumor cells injection. Tumor volumes are measured with an electronic caliper, and effect on tumor growth is calculated. The therapeutic effect, manifested as inhibition of tumor growth, is enhanced upon combination of cyclophosphamide with the blocking antibodies against VSTM5. The frequency of effector T cells=Teffs (CD8⁺ IFNγ⁺) cells and the ratio of T_{eff} and Treg cells are determined in tumor draining lymph nodes and non-draining lymph nodes.

It is anticipated that antibodies which antagonize the immunosuppressive effects of VSTM5 will have at least an additive effect on antitumor immunity when used in combination with a chemotherapeutic such as cyclophosphamide and may elicit as a synergistic benefit as the anti-VSTM5 antibody may render the tumor cells more susceptible to chemotherapy as the antibody should alleviate immunosuppression and may potentiate tumor cell killing mechanisms.

### Example 23

### Anti-Tumor Effect of Immunostimulatory Antibody Against VSTM5 Protein in Combination with Cellular Tumor Vaccines

Therapeutic cancer vaccines enable improved priming of T cells and improved antigen presentation as agents potentiating anti-tumor responses. Among these, are cellular tumor vaccines that use whole cells or cell lysates either as the source of antigens or as the platform in which to deliver the antigens. Dendritic cell (DC)-based vaccines focus on ex vivo antigen delivery to DCs. Other therapeutic cancer vaccines consist of tumor cells genetically modified to secrete immune stimulatory cytokines or growth factors, such as GM-CSF (granulocyte-macrophage colony-stimulating factor) or Flt3-ligand, aim to deliver tumor antigens in vivo in an immune stimulatory context to endogenous DCs.

Several in vivo studies have shown a potent therapeutic effect of immune checkpoint blockade, such as anti-CTLA-4 antibodies, in poorly immunogenic tumors only when combined with GM-CSF or Flt3-ligand-transduced tumor vaccines, termed Gvax and Fvax, respectively (van Elsas et al 1999 J. Exp. Med. 190: 355-366; Curran and Allison 2009 Cancer Res. 69: 7747-7755), and that the antibody alone was effective only in the most immunogenic tumor models in mice. Furthermore, combination of two immunotherapeutic agents, such as anti-CTLA4 and anti-PD-1 blocking antibodies, is more effective in conjunction with therapeutic cancer vaccine, such as Gvax or Fvax (Curran et al 2010 PNAS 107: 4275-4280)

The effect of VSTM5 immunostimulatory antibody in combination with tumor cell vaccine, is tested using irradiated melanoma cells engineered to secrete GMCSF or Flt3-ligand (GVAX or FVAX respectively) in the presence or absence of anti-PD-1 blocking antibody (as described in Curran et al 2010 PNAS 107: 4275-4280). Briefly, mice are injected in the flank i.d. at day 0 with 5×104 B16-BL6 cells and treated on days 3, 6, and 9 with 10⁶ irradiated (150 Gy) gene-modified B16 cells (expressing GMCSF or Flt3-ligand) on the contralateral flank in combination with intraperitoneal administration of 100 ug of anti-VSTM5 immunostimulatory antibody, with or without 100 ug of anti-PD-1 blocking antibody (clone RMP1-14) or anti-PDL-1 blocking antibody (9G2). Isotype Ig is used as negative control. Tumor volumes are measured with an electronic caliper, and effect on tumor growth is calculated. The therapeutic effect, manifested as inhibition of tumor growth, is enhanced upon combination of the blocking antibodies against VSTM5 with the gene modified tumor cell vaccine. Anti-PD-1 or anti-PDL-1 blocking antibodies further enhance this effect. The frequency of effector T cells=Teffs (CD8+IFNγ+) cells and the ratio of Teffs and Tregs are determined in tumor draining lymph nodes and non-draining lymph nodes.

It is anticipated that antibodies which antagonize the immunosuppressive effects of VSTM5 may potentiate the efficacy of cancer vaccines as the anti-VSTM5 antibody may render the tumor cells more susceptible to host immune reactions as the anti-VSTM5 antibody should alleviate immunosuppression and may potentiate antigen-specific tumor cell killing mechanisms.

### Example 24

### Anti-Tumor Effect of Immunostimulatory Antibody Against VSTM5 Protein in Combination with Radiotherapy

Radiotherapy has long been used as anti-cancer therapy because of its powerful anti-proliferative and death-inducing capacities. However, recent preclinical and clinical data indicate that immunogenic cell death may also be an important consequence of ionizing radiation, and that localized radiotherapy can evoke and/or modulate anti-tumor immune responses (Reits et al 2006 J. Exp. Med. 203:1259-1271). Preclinical studies have shown enhanced therapeutic effects in combined treatment of radiotherapy and immunotherapy, including blocking antibodies to immune checkpoints such as CTLA4 and PD-1, in the absence or presence of an additional immunotherapy such as activating anti-4-1BB Abs (Demaria et al 2005 Clin. Can. Res. 11:728-734; Verbruge et al 2012 Can. Res. 72:3163-3174).

The combination of blocking anti-VSTM5 antibodies and radiotherapy will be assessed using a syngeneic 4T1 mammary carcinoma cell line in the BALB/c background (as described in Demaria et al 2005 Clin. Can. Res. 11:728-734). Briefly, 5×10⁴ 4 T1 cells are injected s.c. in the flank of BALB/c mice. Treatment begins when tumors reach an average diameter of 5 mm (65 mm3 in volume). Animal groups include treatment with each modality alone (anti-VSTM5 or radiotherapy) and with the isotype Ig Control, and combination of anti-VSTM5 with radiotherapy, or of Ig Control with radiotherapy. Radiotherapy is delivered to the primary tumor by one or two fractions (48 hrs interval) of 12Gy. Anti-VSTM5 Ab or Ig control are given i.p. at 200 ug, on days 1, 4 and 7 after radiotherapy. In an additional set of experiments, blocking anti-PD-1 mAb (RMP1-14) and activating anti-4-1BB mAb (3E1). Tumor volumes are measured with an electronic caliper, and effect on tumor growth is calculated. The therapeutic effect, manifested as inhibition of tumor growth, is enhanced upon combination of the blocking antibodies against VSTM5 with radiotherapy. Anti-PD-1 blocking antibodies or anti-4-1BB activating Abs, further enhance this effect. The frequency of effector T cells=Teffs (CD8⁺ IFNγ⁺) cells and the ratio of Teffs and Tregs are determined in tumor draining lymph nodes and non-draining lymph nodes.

It is anticipated that antibodies which antagonize the immunosuppressive effects of VSTM5 will have at least an additive effect on antitumor immunity when used in combination with radiotherapy and may elicit as a synergistic benefit as the anti-VSTM5 antibody may render the tumor cells more susceptible to radiation as the antibody should alleviate immunosuppression and may potentiate tumor cell killing mechanisms by the radiotherapy.

### Example 25

### The Effect of VSTM5-ECD-Ig Fusion Protein on TH Differentiation

The effect of VSTM5-ECD-Ig fusion protein on Th differentiation using mouse and human CD4⁺ T cells upon activation under specific Th driving conditions is tested. Murine T cell activation is either antigen-specific or polyclonal. Without wishing to be limited by a single hypothesis, the results of these experimental settings, using mouse or human cells, point to an immunomodulatory effect of VSTM5 on T cells, whereby Th1 and Th17 driven responses (secretion of proinflammatory cytokines and cell proliferation under Th1 and Th17 driving conditions) are inhibited, while secretion of anti-inflammatory cytokines (Th2 derived, and IL-10) are promoted.

It is known that one of the mechanisms by which tumors evade immune surveillance is promotion of a Th2/M2 oriented immune response (Biswas S K, et al., 2010 October; Nature Immunology 11(10):889-96). Thus, without wishing to be limited by a single hypothesis, a neutralizing antibody which suppresses the above demonstrated immunomodulatory effect of VSTM5 (i.e. promotion of Th2 response and inhibition of Th1 response) is beneficial for treatment of cancer.

### Example 26

### Assessment of the Effect of Anti-VSTM5 Antibody on Reversal of the Immunosuppression of Sepsis and Improvement of Survival in an Animal Model of Sepsis

In order to investigate the effect of anti-VSTM5 antibody on sepsis in mice, the CLP (cecal ligation and puncture) model is used to induce polymicrobial peritonitis (as described by Brahmamdam et al 2010 J. Leukoc. Biol. 88:233-240; Zhang et al 2010 Critical Care 14:R220; Inoue et al 2011 Shock 36:38-44). CLP is carried out as follows: C57BL/6 mice are anesthetized and a midline abdominal incision is made. The cecum is mobilized, ligated below the ileocecal valve, and punctured twice with a needle. The abdominal wall is closed in two layers and mice are injected subcutaneously with 1 ml of saline within 30 min after surgery for volume resuscitation Sham-operated mice which did not have their cecum ligated or punctured, serve as control. The anti-VSTM5 antibody is administered intraperitoneally at different doses (ranging from 10 to 100 ug/mouse) 24 hrs before CLP (for preventive mode) or 1.5 hours after CLP surgery, followed by another injection at 24 hrs (for therapeutic mode). Isotype control or saline is used as negative controls. Survival is followed over the subsequent eight days. Effects of the antibody therapy is evaluated also on total splenocyte and blood lymphocyte counts, immune cell subtypes and cytokine production at various time points after surgery. The effect on sepsis-induced lymphocyte apoptosis is evaluated. Treatment with anti-VSTM5 antibody has a beneficial effect on animal survival, and to reduce lymphocyte apoptosis and loss of viable immune cells.

It is anticipated that antibodies which antagonize the immunosuppressive effects of VSTM5 will inhibit or treat sepsis when used alone or in in combination with other actives by alleviating immunosuppression.

### Example 27

### Assessment of the Effect of VSTM5 Alone or in Combination with CTLA4-Ig or Anti-CD154 (CD40L) Antibody on the Enhanced Persistence of AAV-Mediated Gene Therapy

In order to investigate the effect of VSTM5 protein on AAV-mediated gene transfer, the rAAV-Ova model (as described by Adriouch et al 2011 Front. Microbiol. 2:199) is carried out as follows: C57BL/6 mice are injected with 1011 rAAV-Ova vector genomes in 50 ul PBS in the gastrocnemius muscles. Concomitantly, mice are injected i.p. with different doses of VSTM5 protein, without or with combination therapy with 200 ug CTLA4-Ig or with 200 ug anti-CD40L antibody (MR1). Alternatively, VSTM5 protein is administered via gene transfer with rAAV vectors. Blood samples are collected at day 14 and 40 to analyze the percentage of anti-Ova CD8⁺ T cells, the level of anti-Ova IgG and the presence of soluble Ova in the serum. Quantification of soluble Ova concentration in serum is performed by Ova-specific ELISA. Detection of serum anti-Ova IgG antibodies is performed by ELISA using Ova-coated microtiter plates and biotinylated anti-mouse Abs. CD8⁺ T cells that specifically recognize the Ova peptide are detected using PE-conjugated H-2Kb/Ova pentamers. Transduced gastrocnemius muscles are collected at day 40, and levels of Ova DNA and mRNA are quantified by qPCR and qRT-PCR.

### Example 28

### Characterizing Target Cells for VSTM5 Proteins by Determining their Binding Profile to Immune Cells

Splenocytes from D011.10 mice (transgenic mice in which all of the CD4⁺ T cells express a T cell receptor that is specific for OVA323-339 peptide) are activated in the presence of OVA323-339 peptide, and cells are collected at t=0, 6, 12, 24, and 48 hours following initial activation to determine which cell type is expressing a receptor for VSTM5 over time. Cells are then co-stained with VSTM5-ECD-Ig and either for CD3, CD4, CD8, B220, CD19, CD11b, and CD11c.

### Example 29

### Assessment of the Effect of VSTM5 Specific Antibodies on the Ability of B Cells to Class-Switch and Secrete Antibody

Resting B cells are isolated from unprimed C57BL/6 mice and activated in vitro in the presence of anti-CD40 plus (i) no exogenous cytokine, (ii) IL-4, or (iii) IFN-γ. The cell cultures receive control Ig (mIgG2a), anti-CD86 mAb (as a positive control for increased Ig production), or of VSTM5 specific antibodies described herein, at the time of culture set up, and are cultured for 5 days. The VSTM5 specific antibodies are tested at three concentrations each. At the end of culture, supernatants are tested for the presence of IgM, IgG1, and IgG2a via ELISA. If there appears to be an alteration in the ability of the B cells to class-switch to one isotype of antibody versus another, then the number of B cells that have class switched is determined via ELISPOT. If there is an alteration in the number of antibody producing cells, then it is determined if there is an alteration in the level of γ1- and γ2a-sterile transcripts versus the mature transcripts for IgG1 and IgG2a.

### Example 30

### Efficacy of Immunoinhibitory VSTM5 Targeting Antibody in Mouse R-EAE Model of Multiple Sclerosis

The therapeutic effect of immunoinhibitory VSTM5 targeting antibodies for treatment of autoimmune diseases is tested in a mouse model of Multiple Sclerosis; Relapsing Remitting Experimental Autoimmune Encephalomyelitis (R-EAE): Female SJL mice 6 weeks old are purchased from Harlan and maintained in the CCM facility for 1 week prior to beginning the experiment. Mice are randomly assigned into groups of 10 animals and primed with 50 μg PLP139-151/CFA on day 0. Mice receive 6 i.p. injections of 100 ug/dose of immunoinhibitory VSTM5 targeting antibody, mIgG2a isotype control, or CTLA4-Ig (mouse ECD fused to mouse IgG2a Fc) as positive control. Treatments begin at the time of disease induction (preventive mode) or at onset of disease remission (therapeutic mode) and are given 3 times per week for at least 2 weeks. Mice are scored for disease symptoms on a 0-5 disease score scale: 0, no abnormality; 1, limp tail; 2, limp tail and hind limb weakness; 3, hind limb paralysis; 4, hind limb paralysis and forelimb weakness; and 5, moribund.

### Example 31

### Efficacy of Immunoinhibitory VSTM5 Targeting Antibody in Mouse CIA Models of Rheumatoid Arthritis

Immunoinhibitory VSTM5 targeting antibodies are tested in mouse model of collagen-induced arthritis (CIA) which is a model of rheumatoid arthritis. Male DBA/1 mice are housed in groups of 8-10, and maintained at 21° C.±2° C. on a 12 h light/dark cycle with food and water ad libitum. Arthritis is induced by immunization with type II collagen emulsified in complete Freund's adjuvant. Mice are monitored on a daily basis for signs of arthritis. On the appearance of arthritis (day 1) treatment with immunoinhibitory VSTM5 targeting antibodies, mIgG2a isotype control or CTLA4-Ig (mouse ECD fused to mouse IgG2a Fc) as positive control (100 ug/dose, each) is initiated and given 3 times per week for 10 days. Hind footpad swelling is measured (using microcalipers), as well as the number and degree of joint involvement in all four limbs. This yields two measurements, clinical score and footpad thickness that can be used for statistical assessment.

At the end of the treatment period mice are bled and sacrificed. For histological analysis, paws are removed at post mortem, fixed in buffered formalin (10% v/v), then decalcified in EDTA in buffered formalin (5.5% w/v). The tissues are then embedded in paraffin, sectioned and stained with haematoxylin and eosin. The scoring system is as follows: 0=normal; 1=synovitis but cartilage loss and bone erosions absent or limited to discrete foci; 2=synovitis and significant erosions present but normal joint architecture intact; 3=synovitis, extensive erosions, joint architecture disrupted.

The ability of the treatment of mice with established CIA with immunoinhibitory VSTM5 targeting antibodies to result in potent reduction of clinical score, paw swelling and histological damage is tested and compared to the efficacy obtained with CTLA4-Ig.

### Example 32

### Determine Long Term Efficacy of Immunoinhibitory VSTM5 Targeting Antibody in Chronic CIA Model

C57BL/6 mice are treated from onset of disease with immunoinhibitory VSTM5 targeting antibodies, control IgG2a or Enbrel® with 3 doses as in previous studies, in groups of 8-10 mice. At day 10, no further treatment is given and the mice are continuously monitored for 20-30 days in order to establish the time taken for the disease to flare again. This assesses the efficacy of immunoinhibitory VSTM5 targeting antibodies in the chronic CIA model and the duration of its biological effect in rheumatoid arthritis. Long term efficacy is observed in this model. Without being bound by a single hypothesis, a decrease in disease severity is accompanied by decrease in anti-collagen antibody levels as measured for example by ELISA.

### Example 33

### Effect on Tolerance Induction by Immunoinhibitory VSTM5 Targeting Antibody in Transfer Model of CIA

To further understand the effect of immunoinhibitory VSTM5 targeting antibodies on immune regulation, the ability of VSTM5 ECD IG fusion proteins to induce tolerance in a transfer model of arthritis is analyzed.

In brief, spleen and LN cells from arthritic DBA/1 mice treated for 10 days with immunoinhibitory VSTM5 targeting antibodies or control Ig2a are removed and injected i.p into T-cell deficient C.B-17 SCID recipients. The mice then receive an injection of 100 μg type II collagen (without CFA), necessary for successful transfer of arthritis. Arthritis is then monitored in the SCID mice; it is determined that the immunoinhibitory VSTM5 targeting antibodies treatment confers long-term disease protection. Histology is performed and anti-collagen antibody levels are measured to support this determination.

### Example 34

### Assessment of the Effect of VSTM5 Targeting Antibody in a Viral Infection Model of TMEV

Theiler's murine encephalomyelitis virus (TMEV) is a natural endemic pathogen of mice that causes an induced demyelinating disease (TMEV-IDD) in susceptible strains of mice (SJL/J, H-2KS) that resembles the primary progressive form of MS (Munz et al., Nat Rev Immunol 2009; 9:246-58). TMEV infection results in a life-long persistent virus infection of the CNS leading to development of a chronic T cell-mediated autoimmune demyelinating disease triggered via de novo activation of CD4 T cell responses to endogenous myelin epitopes in the inflamed CNS (i.e. epitope spreading) (Miller et al., Nat Med 1997; 3:1133-6; Katz-Levy et al., J Clin Invest 1999; 104:599-610).

SJL mice clear the majority of the virus within 21 days post infection, however a latent viral infection is maintained and infect microglia, astrocytes, and neurons. Disease symptoms are manifested around day 25-30 post infection.

The effect of treatment with VSTM5 targeting antibodies on acute and chronic phases of viral infection is studied in the TMEV-IDD model by assessment of viral clearance and disease severity.

Method:

Female SJL/J mice (5-6 weeks) are infected with TMEV by intracranial inoculation in the right cerebral hemisphere of 3×10⁷ plaque forming units (PFU) of the BeAn strain 8386 of TMEV in 30 ul serum-free medium. From day 2 post infection mice are treated with Control Ig, VSTM5 targeting antibodies, at 100 ug/dose each; 3 doses/week for 2 weeks.

Mice are followed for clinical scoring. On day 7 and day 14 post infection (after 3 and 6 treatments respectively) brains and spinal cords are collected from 5 mice in each treatment group for plaque assays. The tissues are weighted so that the ratio of PFU/mg of CNS tissue could be calculated after the plaque assay is completed.

TMEV Plaque Assay:

Brains and spinal cords of mice treated with Control Ig (mouse IgG2a), or with VSTM5 targeting antibodies are collected at days 7 and 14 post-infection from non-perfused anesthetized mice. The Brains and spinal cords are weighed, and homogenized. CNS homogenates are serially diluted in DMEM and added to tissue culture-treated plates of confluent BHK-21 cells for 1 h incubation at room temperature, with periodic gentle rocking.

A media/agar solution is mixed 1:1 (volume:volume), added to cells and allowed to solidify at room temperature. The plates are then cultured at 34deg C. for 5 days. At the end of culture, 1 ml of formalin is added and incubated at room temperature for 1 h to fix the BHK monolayer. The formalin is poured off into a waste container, and the agar is removed from the plates. Plaques are visualized by staining with crystal violet for 5 min, and plates are gently rinsed with diH2O. To determine PFU/ml homogenate, the number of plaques on each plate is multiplied by the dilution factor of the homogenate and divided by the amount of homogenate added per plate. The PFU/ml is divided by the weight of the tissue to calculate PFU/mg tissue.

### Example 35

### Assessment of the Effect of VSTM5 Targeting Antibody on Primary and Secondary Immune Response to Viral Infection in a Mouse Model of Influenza

To test the effect of VSTM5 targeting antibodies on primary and secondary immune responses to viral infection, BALB/c naïve mice (for primary immune responses) and ‘HA-memory mice’, is used, as well as ‘polyclonal flu-memory mice’ (to assess secondary responses mediated by memory CD4 T cells), which are generated as detailed in Teijaro et al., J Immunol. 2009: 182; 5430-5438, and described below.

To obtain ‘HA-memory mice’, first HA-specific memory CD4 T cells are generated, naive CD4 T cells are purified from spleens of HA-TCR mice [BALB/c-HA mice which express transgenic T cell receptor (TCR) specific for influenza hemagglutinin (HA) peptide (110-119)] and primed in vitro by culture with 5.0 μg/ml HA peptide and mitomycin C-treated, T-depleted BALB/c splenocytes as APCs for 3 days at 37° C. The resultant activated HA-specific effector cells are transferred into congenic BALB/c (Thy1.1) hosts (5×10⁶ cells/mouse) to yield “HA-memory mice” with a stable population of HA-specific memory CD4 T cells.

To obtain ‘polyclonal-memory mice’, first polyclonal influenza-specific memory CD4 T cells are generated, by infecting BALB/c mice intranasally with a sublethal dose of PR8 influenza, CD4 T cells are isolated 2-4 months postinfection, and the frequency of influenza-specific memory CD4 T cells is determined by ELISPOT. CD4 T cells from previously primed mice are transferred into BALB/c hosts to generate “polyclonal flu-memory” mice with a full complement of endogenous T cells.

Primary and secondary responses to influenza virus are tested by infecting naïve BALB/c mice or BALB/c-HA memory mice and BALB/c ‘polyclonal flu-memory mice’ with sublethal or lethal doses of PR8 influenza virus by intranasal administration.

Mice are treated with VSTM5 targeting antibodies or with mIgG2a control before and following influenza challenge. Weight loss and mortality will be monitored daily. Six days after the challenge, viral content in the bronchoalveolar lavage (BAL) is analyzed by collecting lavage liquid and testing the supernatant for viral content by determining the tissue culture infectious dose 50% (TCID50) in MDCK cells. In addition, lung tissue histopathology is performed.

To test the effect VSTM5 targeting antibodies on T cell expansion BALB/c or BALB/c-HA memory mice or BALB/c ‘polyclonal flu-memory mice’ are infected as above and administered with BrdU (1 mg/dose) on days 3, 4 and 5 post infection. On day 6, spleen and lung are harvested and BrdU incorporation is estimated. Cytokine production by lung memory CD4 T cells during influenza challenge is also studied in HA-specific memory CD4 T cells stimulated in vitro with HA peptide in the presence VSTM5 targeting antibodies or with IgG2a for 18 hours.

### Example 36

### Assessment of the Effect of VSTM5 Targeting Antibodies on Primary and Secondary CD8⁺ T Cell Response to Viral Infection in a Mouse Model of Influenza

The effect of VSTM5 targeting antibodies on primary CD8⁺ T cell responses to influenza virus is studied according to methods as described in the literature (Hendriks et al., J Immunol 2005; 175; 1665-1676; Bertram et al., J Immunol. 2004; 172:981-8) using C57BL/6 mice infected with influenza A HKx31 by intranasal or intraperitoneal administration. VSTM5 targeting antibodies or mIgG2a control are administered during priming. Animal weight loss and mortality is monitored daily. To follow virus-specific CD8⁺ T cells, MHC H-2Db tetramers loaded with the major CD8 T cell epitope, the NP366-374 peptide are used. Virus-specific H-2Db/NP366-374⁺CD8⁺ T cells in the lung, draining lymph nodes, and spleen are expected to reach a peak around day 8-10 post infection and decline thereafter to only 1.5% virus-specific CD8⁺ T cells (Hendriks et al J Immunol 2005; 175; 1665-1676; Bertram et al., J Immunol. 2002; 168:3777-85; Bertram et al., J Immunol. 2004; 172:981-8). Thus, mice are sacrificed at days 8 and 21 post infection, and virus-specific CD8⁺ T cell numbers is evaluated in the lung, draining lymph nodes and spleen. Viral clearance is assessed. CD8⁺ T cell responses are evaluated in spleen cell suspensions, and include intracellular IFN-γ staining and CTL activity, as previously described (Bertram et al., J Immunol. 2004; 172:981-8) and detailed below.

Cells are surface-stained with FITC-conjugated anti-mouse CD62L, PE-conjugated anti-mouse CD8 to measure CD8⁺ activated T cells (or anti-mouse CD4 to follow CD4⁺ cells). In addition to these Abs, allophycocyanin-labeled tetramers consisting of murine class I MHC molecule H-2Db, β2-microglobulin, and influenza NP peptide, NP366-374 are used to measure influenza-specific CD8⁺ T cells. For intracellular IFN-γ staining, cell suspensions are restimulated in culture medium for 6 h at 37° C. with 1 μM NP366-374 peptide and Golgi Stop (BD PharMingen, San Diego, Calif.). Cells are then harvested, resuspended in PBS/2% FCS/azide, and surface stained with PE-anti-CD8 and FITC-anti-CD62L as described above. After surface staining, cells will be fixed in Cytofix/Cytoperm solution (BD PharMingen) and then stained with allophycocyanin-conjugated antimouse IFN-γ diluted in 1× perm/wash solution (BD PharMingen). Samples are analyzed by Flow Cytometry.

For cytotoxicity assays (CTL responses) splenocytes from influenza-infected mice are incubated for 2 h at 37° C. to remove adherent cells. Serial 3-fold dilutions of effectors are assayed for anti-influenza NP366-374-specific CTL activity against ⁵¹Cr-labeled EL4 cells pulsed with 50 μM NP366-374 peptide for 6 h as described by Bertram et al 2002 and Bertram et al 2004.

At 3 weeks postinfection, some mice are rechallenged with the serologically distinct influenza A/PR8/34 (PR8), which shares the NP gene with influenza A HKx31, but differs in hemagglutinin and neuraminidase, so that neutralizing Abs do not limit the secondary CTL response. Mice are sacrificed at days 5 & 7 following virus rechallenge, and virus-specific CD8⁺ T cell numbers is evaluated in the lung, draining lymph nodes and spleen as described by Hendriks et al and Bertram et al (Hendriks et al., J Immunol 2005; 175; 1665-1676; Bertram et al., J Immunol. 2004; 172:981-8) and detailed above. Secondary CD8⁺ T cell responses, including intracellular IFN-γ staining and CTL activity, are evaluated in spleen cell suspensions of mice at days 5 & 7 following virus rechallenge, as described above.

To determine the effect of VSTM5 targeting antibodies on expansion and accumulation of memory CD8⁺ T cells during the secondary response, adoptive transfer experiments are performed, according to methods previously described (Hendriks et al., J Immunol 2005; 175; 1665-1676; Bertram et al., J Immunol. 2004; 172:981-8): mice are immunized with influenza A HKx31. Twenty-one days later, T cells are purified from spleens on mouse T cell enrichment immunocolumns (Cedarlane Laboratories, Hornsby, Ontario, Canada) and labeled with CFSE (alternatively Thy1.1 congenic mice are used as recipients). Equal numbers of tetramer-positive T cells are injected through the tail vein of recipient mice. Mice are rechallenged with influenza virus as described above, and 7 days later splenocytes are evaluated for donor virus-specific CD8⁺ T cells, as detailed above.

### Example 37

### Assessment of Protein Expression in Exhausted T Cells, and the Binding and Effect of VSTM5 Targeting Antibodies on Reversing Exhausted T Cell Phenotype

Memory CD8⁺ T-cell differentiation proceeds along distinct pathways after an acute versus a chronic viral infection (Klenerman and Hill Nat Immunol 6, 873-879, 2005). Memory CD8⁺ T cells generated after an acute viral infection are highly functional and constitute an important component of protective immunity. In contrast, chronic infections are often characterized by varying degrees of functional impairment of virus-specific T-cell responses, and this defect is a principal reason for the inability of the host to eliminate the persisting pathogen. Although functional effector T cells are initially generated during the early stages of infection, they gradually lose function during the course of the chronic infection leading to exhausted phenotype characterized by impaired T cell functionality.

Effect of VSTM5 Targeting Antibodies on Clearance of Viral Infection and on T Cell Functions During Acute and Chronic Viral Infection.

In these experiments the effect of VSTM5 targeting antibodies on acute and chronic viral infection is evaluated in a mouse model of infection with LCMV (lymphocytic chroriomeningitis virus) according to methodology described by Wherry et al J. Virol. 77: 4911-4927, 2003 and Barber et al Nature, 2006.

In this model two LCMV strains which can cause either acute or chronic infections in adult mice are used; the Armstrong strain which is cleared within a week, and the clone 13 strain which establishes a persistent infection that can last for months. As these two strains differ in only two amino acids, preserving all known T cell epitopes, it is possible to track the same CD8⁺ T cell responses after an acute or chronic viral infection. In contrast to the highly robust memory CD8⁺ T cells generated after an acute Armstrong infection, LCMV-specific CD8⁺ T cells become exhausted during a persistent clone 13 infection (Wherry et al J. Virol. 77: 4911-4927, 2003; Barber et al., Nature 2006; 439:682-7).

Mice are infected with 2×10⁵ PFU of Armstrong strain of LCMV intraperitoneally to initiate acute infection or 2×10⁶ PFU of C1-13 intravenously to initiate chronic infection. Mice are treated i.p. with VSTM5 targeting antibodies or with mIgG2a control, or an isotype control.

The mice are monitored for numbers of virus specific CD8⁺ T cells in the spleen, using virus-specific MHC tetramer epitopes, such as DbNP396-404 and DbGP33-41 which differ in acute or chronic infections. CD8⁺ T cell functional assays, such as intracellular cytokines levels and CTL activity, are carried out as described by Wherry et al J. Virol. 77: 4911-4927, 2003. Additional assays include production by splenocytes after stimulation with virus specific epitopes; and assessment of viral titers in the serum and in the spleen, liver, lung and kidney (Wherry et al J. Virol. 77: 4911-4927, 2003; Barber et al., Nature. 2006; 439:682-7).

Assessment of VSTM5 Expression on Exhausted T Cells and Binding of VSTM5 Targeting Antibodies to Exhausted T Cells

In this experiment VSTM5 expression is detected on exhausted T cells and the binding of VSTM5 targeting antibodies to exhausted T cells is effected in order to evaluate regulation of these proteins or their counterpart receptors during exhaustion of T cells. In the experiments T cells are isolated from mice with chronic LCMV infection induced with C1-13 strain. The cells are co-stained with fluorescently labeled anti-PD-1 Ab as positive control (PD-1 is highly expressed by exhausted T cells) and biotinylated VSTM5 ECD Ig fusion proteins or biotinylated VSTM5 targeting antibodies, and respective isotype control. Binding is detected by FACS analysis using fluorescently labeled streptavidin.

### Example 38

### Assessment of VSTM5 Protein Expression in Follicular Helper T (Tfh) Cells and Binding of Ig Fusion Proteins to Tfh Cells

Follicular helper T (Tfh) cells are a subset of CD4⁺ T cells specialized in B cell help (reviewed by Crotty, Annu. Rev. Immunol. 29: 621-663, 2011). Tfh cells migrate into B cell follicles within lymph nodes, and interact with cognate B cells at the T cell-B cell border and subsequently induce germinal center B cell differentiation and germinal center formation within the follicle (Reviewed by Crotty, Annu. Rev. Immunol. 29: 621-663, 2011). The requirement of Tfh cells for B cell help and T cell-dependent antibody responses, indicates that this cell type is of great importance for protective immunity against various types of infectious agents, as well as for rational vaccine design.

Tfh cells are readily identifiable at the peak of the CD4⁺ T cell response to an acute lymphocytic choriomeningitis virus (LCMV) infection as CXCR5^{hi}SLAM^{lo} BTLA^{hi}PD1^{hi}Bcl6⁺ virus-specific CD4⁺ T cells (Choi et al 2011, Immunity 34: 932-946). T cells are isolated from mice with acute LCMV infection induced with 2×10⁵ PFU of Armstrong strain of LCMV administered intraperitoneally. The cells are co-stained with fluorescently labeled antibodies for markers of Tfh (CXCR5, PD1, BTLA, Bcl6) which are highly expressed by Tfh cells, and biotinylated VSTM5-ECD-Ig fusion proteins or biotinylated antibodies specific for VSTM5 and respective isotype controls. Binding of Fc fused protein or antibody is detected by FACS analysis using fluorescently labeled streptavidin.

### Example 39

### Assessment of the Effect of VSTM5 Targeting Antibodies on Follicular Helper T (Tfh) Cells Generation and Activity

In order to investigate the effect of VSTM5 targeting antibodies on Tfh differentiation and development of B cell immunity in vivo, C57BL/6 are treated with VSTM5 targeting antibodies and an isotype control throughout the course of an acute viral infection with Armstrong strain of LCMV (lymphocytic choriomeningitis virus). Tfh differentiation and Bcl6 protein expression is assessed by FACS analysis as described by Eto et al 2011 (PLoS One 6: e17739). Splenocytes are analyzed 8 days following LCMV infection, Tfh generation (CD44^{hi}CXCR5^{hi}SLAMlo) and Bcl6 expression is evaluated by FACS analysis. In addition, the effect of VSTM5 targeting antibodies) on antigen-specific B cell responses is evaluated as described by Eto et al 2011 (PLoS One 6: e17739), including titers of anti-LCMV IgG in the serum at 8 days following LCMV infection, and quantitation by FACS analysis of plasma cell (CD138⁺IgD⁻) development at 8 days post-infection, gated on CD19⁺ splenocytes.

### Example 40

### Effect of Immunoinhibitory VSTM5 Targeting Antibodies in Modulation of Type 1 Diabetes in NOD Mice, CD28-KO NOD, and B7-2-KO NOD

Effect of Immunoinhibitory VSTM5 Targeting Antibodies in NOD Mice

The effects of VSTM5 antibodies is tested in a widely used mouse model of type 1 diabetes: nonobese diabetic (NOD) mice. These mice spontaneous develop spontaneous insulitis, the hallmark pathologic lesion, which evolves through several characteristic stages that begin with peri-insulitis and end with invading and destructive insulitis and overt diabetes. Peri-insulitis is first observed at 3-4 wk of age, invading insulitis at 8-10 wk, and destructive insulitis appears just before the onset of clinical diabetes, with the earliest cases at 10-12 wk. At 20 wk of age, 70-80% of female NOD mice become diabetic (Ansari et al 2003 J. Exp. Med. 198: 63-69).

Two KO mice: CD-28-KO NOD mice and B7-1/B7-2 double KO NOD mice,—which develop accelerated diabetes (Lenschow et al 1996 Immunity 5: 285-293; Salomon et al 2000 Immunity 12: 431-440), are also used.

NOD Mice Treated with Immunoinhibitory VSTM5 Targeting Antibodies Early and Late Phases During the Evolution of Diabetes, Before or after Disease Onset

In this study, NOD mice are treated with immunoinhibitory VSTM5 targeting antibodies early and late phases during the evolution of diabetes, before or after disease onset, in order to examine the effects of these compounds on disease pathogenesis and to demonstrate that such treatment reduces disease onset and ameliorates pathogenesis. To study the effect on insulitis, blood glucose levels are measured 3 times/week, for up to 25 weeks (Ansari et al 2003 J. Exp. Med. 198: 63-69).

Mechanism of disease modification and mode of action is studied by experimental evaluation of individual immune cell types: pancreas, pancreatic LNs and spleen will be harvested to obtain Tregs, Th subtypes and CD8⁺ T cells, DCs and B cells. Effect on cytokines secretion from cells isolated from pancreas, pancreatic LN and spleen is analyzed, focused on IFNγ, IL-17, IL-4, IL-10 and TGFβ. Upon effect of the tested compounds, the mechanism of disease modification is studied by examination of individual immune cell types (including Tregs, Th subtypes and CD8⁺ T cells, DCs and B cells); cytokines (IFNγ, IL-17, IL-4, IL-10 and TGFβ) and histology. Histological analysis of the pancreas is carried out to compare the onset of insulitis, and the lymphocyte infiltration.

It is anticipated based on the immunosuppressive effects of VSTM5 that Immunoinhibitory VSTM5 targeting antibodies which agonize or mimic the effects of VSTM5 on immunity will prevent or reduce disease onset or the severity thereof in the above studies.

Effect of Immunoinhibitory VSTM5 Targeting Antibodies in Modulation of Type 1 Diabetes in Adoptive Transfer Model

To further investigate the mode of action of the immunoinhibitory VSTM5 targeting antibodies in adoptive transfer model of diabetes is used. T cells from diabetic or prediabetic NOD donors are transferred to NOD SCID recipient mice. These mice are monitored for development of diabetes. The urine glucose and blood glucose, and assess histology of the pancreas, and T cell responses are monitored as described in the previous example. It is anticipated based on the immunosuppressive effects of VSTM5 that Immunoinhibitory VSTM5 targeting antibodies which agonize or mimic the effects of VSTM5 on immunity will prevent or reduce disease onset or the severity thereof in the above studies.

It is anticipated based on the immunosuppressive effects of VSTM5 that Immunoinhibitory VSTM5 targeting antibodies which agonize or mimic the effects of VSTM5 on immunity will prevent or reduce disease onset or the severity thereof in the above studies.

Transfer Diabetes Model

In this experiment diabetes is induced by the transfer of activated CD4⁺CD62L⁺CD25⁻ BDC2.5 T cells (transgenic for TCR recognizing islet specific peptide 1040-p31 activated by incubation with 1040-p31) to NOD recipients. Mice are treated with immunoinhibitory VSTM5 targeting antibodies, control mIgG2a or positive control. Treatments begin 1 day following transfer. Mice are followed for glucose levels 10-28 days post transfer (Bour-Jordan et al., J Clin Invest. 2004; 114(7):979-87).

Seven days post treatment pancreas, spleen, pancreatic LN and peripheral lymph node cells are extracted and examined for different immune cell populations. In addition, recall responses are measured by testing ex-vivo proliferation and cytokine secretion in response to p31 peptide.

It is anticipated based on the immunosuppressive effects of VSTM5 that Immunoinhibitory VSTM5 targeting antibodies which agonize or mimic the effects of VSTM5 on immunity will prevent or reduce disease onset or the severity thereof in the above studies.

### Example 41

### Effect of Immunoinhibitory VSTM5 Targeting Antibodies in Lupus Mouse Models

Lupus-Prone Mouse Model, (NZB×NZW)F1 (B/W)

An experiment is conducted using the lupus-prone mouse model, (NZB×NZW)F1 (B/W). Cyclophosphamide (CTX) is the primary drug used for diffuse proliferative glomerulonephritis in patients with renal lupus, Daikh and Wofsy reported that combination treatment with CTX and CTLA4-Ig was more effective than either agent alone in reducing renal disease and prolonging survival of NZB/NZW F1 lupus mice with advanced nephritis (Daikh and Wofsy, J Immunol, 166(5):2913-6 (2001)). In the proof-of-concept study, treatments with immunoinhibitory VSTM5 targeting antibodies and CTX either alone or in combination are tested.

Blood samples are collected 3 days before the protein treatment and then every other week during and after treatments for plasma anti-dsDNA autoantibody analysis by ELISA. Glomerulonephritis is evaluated by histological analysis of kidneys. Proteinuria is measured by testing fresh urine samples using urinalysis dipsticks. It is anticipated that the results of these experiments will demonstrate that immunoinhibitory VSTM5 targeting antibodies have a beneficial effect in at least ameliorating lupus nephritis.

NZM2410-Derived B6.Sle1.Sle2.Sle3 Mouse Model of SLE

An experiment is conducted using the NZM2410-derived B6.Sle1.Sle2.Sle3 mouse model of SLE. NZM2410 is a recombinant inbred strain produced from NZB and NZW that develops a highly penetrant lupus-like disease with an earlier onset of disease (Blenman et al 2006 Lab. Invest. 86: 1136-1148). The effect of immunoinhibitory VSTM5 targeting antibodies is studied in this model by assessment of proteinuria and autoantibodies as described above.

Induced Lupus Model

Another lupus study is effected using the induced lupus model. This model is based on chronic graft-vs-host (cGVH) disease induced by the transfer of Ia-incompatible spleen cells from one normal mouse strain (such as B6.C-H2(bm12)/KhEg (bm12)) to another (such as C57BL/6), which causes an autoimmune syndrome resembling systemic lupus erythematosus (SLE), including anti-double-stranded DNA (anti-dsDNA) autoantibodies and immune complex-type proliferative glomerulonephritis (Appleby et al Clin. Exp. Immunol. 1989 78: 449-453); Eisenberg and Choudhury 2004 Methods Mol. Med. 102:273-284).

Lupus is induced in this model following injection of spleen cells from bm12 mice into C57BL/6 recipients. The effect of immunoinhibitory VSTM5 targeting antibodies is studied in this model by assessment of proteinuria and autoantibodies as described above. T cell and responses B cell responses will also be evaluated.

Study IV: The MRL/lpr lupus prone mouse model is used. The effect of immunoinhibitory VSTM5 targeting antibodies is studied in this model by assessment of proteinuria and autoantibodies as described above.

### Example 42

### Effect of Immunoinhibitory VSTM5 Targeting Antibodies in the Control of Intestinal Inflammation

An adoptive transfer mouse model of colitis in mice is used, whereby transfer of CD45RB^{high}-CD4⁺ naïve T cells from BALB/c mice to syngeneic SCID mice leads to the development of an IBD-like syndrome by 6-10 wks after T cell reconstitution, similar to human Crohn's disease.

SCID mice are reconstituted by i.p. injection of syngeneic CD45RB^{high-}CD4⁺ T cells either alone or cotransferred with syngeneic CD45R^{Blow-}CD4⁺ or CD25⁺CD4⁺ cells (4×10⁵/mouse of each cell population) (Liu et al., J Immunol. 2001; 167(3): 1830-8). Colitic SCID mice, reconstituted with syngeneic CD45RB^{high}CD4⁺ T cells from spleen of normal mice, are treated i.p. with immunoinhibitory VSTM5 targeting antibodies or Ig isotype control, twice a week starting at the beginning of T cell transfer up to 8 wk. All mice are monitored weekly for weight, soft stool or diarrhea, and rectal prolapse. All mice are sacrificed 8 wk after T cell transfer or when they exhibit a loss of 20% of original body weight. Colonic tissues are collected for histologic and cytologic examinations. The anticipated results should demonstrate that immunoinhibitory VSTM5 targeting antibodies have a beneficial effect in ameliorating inflammatory bowel disease.

### Example 43

### Effect of Immunoinhibitory VSTM5 Targeting Antibodies in Mouse Model of Psoriasis

Establishment of Psoriasis SCID Xenograft Model.

Human psoriasis plaques are transplanted on to the SCID mice. Shave biopsies (2.5×2.5 cm) are taken from patients with generalized plaque psoriasis involving 5-10% of the total skin that did not receive any systemic treatment for psoriasis or phototherapy for 6 months and did not receive any topical preparations other than emollients for 6 weeks. The biopsies are obtained from active plaques located on the thigh or arm. Each piece of biopsy is divided into four equal parts of approximately 1 cm² size. Each piece is transplanted to a separate mouse.

Under general anesthesia, a graft bed of approximately 1 cm² is created on the shaved area of the back of a 7- to 8-week-old CB17 SCID mouse by removing a full-thickness skin sample, keeping the vessel plexus intact on the fascia covering the underlying back muscles. The partial thickness human skin obtained by shave biopsy is then orthotopically transferred onto the graft bed. Nexaband, a liquid veterinary bandage (Veterinary Products Laboratories, Phoenix, Ariz.) is used to attach the human skin to the mouse skin and an antibiotic ointment (bacitracin) is applied. Mice are treated intraperitoneally three times per week for 4 weeks with immunoinhibitory VSTM5 targeting antibodies, isotype control or CTLA4-Ig (positive control).

Punch biopsies (2 mm) are obtained on day 0 (before treatment) and day 28 (after treatment) of the study period. Biopsies are snap frozen and cryosections for histopathological and immunohistochemical studies. Therapeutic efficacy is determined by comparing pre- and post-treatment data: (i) rete peg lengths to determine the effect on epidermal thickness and (ii) the level of lymphomononuclear cell infiltrates to determine the effect on inflammatory cellular infiltrates. (Raychaudhuri et al. 2008, J Invest Dermatol.; 128(8):1969-76; Boehncke et al., 1999 Arch Dermatol Res 291:104-6). It is anticipated that the results will demonstrate that immunoinhibitory VSTM5 targeting antibodies have a beneficial effect in ameliorating psoriasis.

Effect of VSTM5 in Psoriasis and Colitis Model by Adoptive Transfer of CD45RB^{hi}CD4⁺ T Cells in SCID Mice Immunocompromised mice are injected intravenously (i.v.) with 0.3_10⁶ CD4⁺ CD45RBhi cells. On the day following the adoptive transfer of cells, mice are injected intraperitoneally (i.p.) with 10 μg of staphylococcal enterotoxin B (Davenport et al., Int. Immunopharmacol. 2002 April; 2(5):653-72). Recipient mice are treated with immunoinhibitory VSTM5 targeting antibodies, isotype control or CTLA4-Ig (positive control). Mice are evaluated once a week for 8 weeks for weight loss and presence of skin lesions. It is anticipated that the results of this experiment will be similar to those described above.

### Example 44

### Effect of Immunoinhibitory VSTM5 Targeting Antibodies in Modulating Transplant Rejection

Effect of VSTM5 in a model of allogeneic islet transplantation in diabetic mice. To test the effect of immunoinhibitory VSTM5 targeting antibodies on transplant rejection, a model of allogeneic islet transplantation is used. Diabetes is induced in C57BL/6 mice by treatment with streptozotocin. Seven days later, the mice are transplanted under the kidney capsule with pancreatic islets which are isolated from BALB/c donor mice. Recipient mice are treated with immunoinhibitory VSTM5 targeting antibodies or with mIgG2a as a negative control. Tolerance with ECDI-fixed donor splenocytes is used as the positive control for successful modulation islet graft rejection. Recipient mice are monitored for blood glucose levels as a measure of graft acceptance/rejection (Luo et al., PNAS, Sep. 23, 2008 105(38) 14527-14532).

Effect of VSTM5 in the Hya-Model of Skin Graft Rejection.

In humans and certain strains of laboratory mice, male tissue is recognized as non-self and destroyed by the female immune system via recognition of histocompatibility-Y chromosome encoded antigens (Hya). Male tissue destruction is thought to be accomplished by cytotoxic T lymphocytes in a helper-dependent manner.

To test the effect of immunoinhibitory VSTM5 targeting antibodies on transplantation, the Hya model system is used, in which female C57BL/6 mice receive tail skin grafts from male C57BL/6 donors.

In this study, female C57BL/6 mice are engrafted with orthotopic split-thickness tail skin from age matched male C57BL/6 mice. The mice are treated with immunoinhibitory VSTM5 targeting antibodies s, isotype control mIgG2a. Immunodominant Hya-encoded CD4 epitope (Dby) attached to female splenic leukocytes (Dby-SP) serve as positive control for successful modulation of graft rejection (Martin et al., J Immunol. 2010 Sep. 15; 185(6): 3326-3336). Skin grafts are scored daily for edema, pigment loss and hair loss. Rejection is defined as complete hair loss and more than 80% pigment loss.

In addition, T cell recall responses of cells isolated from spleens and draining lymph nodes at different time points are studied in response to CD4 specific epitope (Dby), CD8 epitopes (Uty and Smcy) or irrelevant peptide (OVA 323-339) while anti CD3 stimulation is used as positive control for proliferation and cytokine secretion.

Effect of Immunoinhibitory VSTM5 Targeting Antibodies on Graft Rejection

The effect of immunoinhibitory VSTM5 targeting antibodies on graft rejection is further studied in a murine model of syngeneic bone marrow cells transplantation using the Hya model system described above. Male hematopoietic cells expressing the CD45.1 marker are transplanted to female host mice which express the CD45.2 congenic marker. Female hosts are treated with immunoinhibitory VSTM5 targeting antibodies or with isotype control mIgG2a. The female hosts are followed over time and the presence of CD45.1⁺ cells is monitored.

The invention has been described and various embodiments provided relating to manufacture and selection of desired anti-VSTM5 antibodies for use as therapeutics and diagnostic methods for various diseases. Different embodiments and sub-embodiments may optionally be combined herein in any suitable manner, beyond those explicit combinations and sub combinations shown herein. The invention is now further described by the claims which follow.

1-319. (canceled)

320) An anti-VSTM5 antibody or an antigen-binding fragment thereof which specifically binds to the polypeptide of SEQ ID NO: 2, 3, 6, 7, 132, 349, or to a polypeptide possessing at least 90% sequence identity therewith or to a non-human VSTM5 ortholog, wherein such antibody or antigen-binding fragment inhibits, antagonizes or blocks at least one effect that a VSTM5 polypeptide having the amino acid sequence of SEQ ID NO: 2, 3, 6, 7, 132, 349 elicits on immunity or on one or more types of immune cells, which mediates any combination of at least one of the following immunostimulatory effects on immunity: (i) increases immune response, (ii) increases T cell activation, (iii) increases cytotoxic T cell activity, (iv) increases NK cell activity, (v) alleviates T-cell suppression, (vi) increases pro-inflammatory cytokine secretion, (vii) increases IL-2 secretion; (viii) increases interferon-γ production, (ix) increases Th1 response, (x) decrease Th2 response, (xi) decreases or eliminates cell number and/or activity of at least one of regulatory T cells (Tregs), myeloid derived suppressor cells (MDSCs), iMCs, mesenchymal stromal cells, TIE2-expressing monocytes, (xii) reduces regulatory cell activity, and/or the activity of one or more of myeloid derived suppressor cells (MDSCs), iMCs, mesenchymal stromal cells, TIE2-expressing monocytes, (xiii) decreases or eliminates M2 macrophages, (xiv) reduces M2 macrophage pro-tumorigenic activity, (xv) decreases or eliminates N2 neutrophils, (xvi) reduces N2 neutrophils pro-tumorigenic activity, (xvii) reduces inhibition of T cell activation, (xviii) reduces inhibition of CTL activation, (xix) reduces inhibition of NK cell activation, (xx) reverses T cell exhaustion, (xxi) increases T cell response, (xxii) increases activity of cytotoxic cells, (xxiii) stimulates antigen-specific memory responses, (xxiv) elicits apoptosis or lysis of cancer cells, (xxv) stimulates cytotoxic or cytostatic effect on cancer cells, (xxvi) induces direct killing of cancer cells, (xxvii) increases Th17 activity and/or (xxviii) induces complement dependent cytotoxicity and/or antibody dependent cell-mediated cytotoxicity.

321) The anti-VSTM5 antibody or an antigen-binding fragment thereof of claim 1 which comprises an antigen-binding region that binds specifically to (i) a first polypeptide having an amino acid sequence set forth in any of SEQ ID NOs:1, 12-21, or to a polypeptide possessing at least 90, 95, 96, 97, 98 or 99% sequence identity therewith or to the same region of a non-human VSTM5 ortholog, and (ii) wherein a second polypeptide having an amino acid sequence set forth in any of SEQ ID NOs: 2, 3, 6, 7, 132, 349 or a polypeptide possessing at least 90, 95, 96, 97, 98 or 99% sequence identity therewith or a non-human VSTM5 ortholog which comprises said first polypeptide, and (iii) with the further proviso that said antigen-binding region does not specifically bind to any other portion of said second polypeptide apart from said first polypeptide.

322) The antibody or an antigen-binding fragment according to claim 321, which is selected from a chimeric, human, primatized, bispecific or humanized antibody which optionally has an in vivo half-life of at least 1 week, 2 weeks, 3 weeks or a month.

323) The anti-VSTM5 antibody or an antigen-binding fragment thereof according to claim 320, which is coupled to a therapeutic moiety, detectable moiety, or a moiety that alters (increases or decreases) in vivo half-life.

324) The anti-VSTM5 antibody or an antigen-binding fragment thereof according to claim 323, which is coupled to a therapeutic agent selected from a drug, a radionuclide, a fluorophore, an enzyme, a toxin, or a chemotherapeutic agent; and/or a detectable marker selected from a radioisotope, a metal chelator, an enzyme, a fluorescent compound, a bioluminescent compound or a chemiluminescent compound.

325) The anti-VSTM5 antibody or an antigen-binding fragment according to claim 320 which binds human or murine VSTM5 with a binding affinity (KD) no more than 500 nM as determined by any of the binding affinity methods disclosed herein or alternatively or additionally which binds human or murine VSTM5 with a binding affinity (KD) of about 10⁻⁵, 10⁻⁶, 10⁻⁷, 10⁻⁸, 10⁻⁹, 10⁻¹⁰, 10⁻¹¹, 10⁻¹²M or less as determined by any of the binding affinity methods disclosed herein; or alternatively or additionally which binds human or murine VSTM5 with a binding affinity (KD) no more than 50 nM as determined by any of the binding affinity methods disclosed herein.

326) The antibody antigen-binding fragment thereof, according to claim 320, which promotes CTL activity, wherein CTL activity includes the secretion of one or more proinflammatory cytokines and/or CTL mediated killing of target cells; and/or which promotes CD4+ T cell activation and/or CD4+ T cell proliferation and/or CD4+ T cell mediated cell depletion; and/or which promotes CD8+ T cell activation and/or CD8+ T cell proliferation and/or CD8+ T cell mediated cell depletion; and/or which enhances NK cell activity, and/or NK cell proliferation and/or NK cell mediated cell depletion, wherein enhanced NK cell activity includes increased depletion of target cells and/or proinflammatory cytokine release; and/or which decreases or eliminates the differentiation, proliferation and/or activity of regulatory cells (Tregs), and/or the differentiation, proliferation, infiltration and/or activity of myeloid derived suppressor cells (MDSCs); and/or which decreases or eliminates the infiltration of inducible Tregs (iTregs) into a target site.

327) The antibody or an antigen-binding fragment thereof according to claim 326, wherein said target site is a cancer cell, tissue or organ, tumor draining lymph node, or an infectious disease site or lesion.

328) The antibody or antigen-binding fragment of claim 320 which promotes anti-tumor immunity by suppressing one or more of the effects of VSTM5 on immunity; and/or which promotes an immune response against an infectious agent by suppressing one or more of the effects of VSTM5 on immunity.

329) The anti-VSTM5 antibody or the antigen-binding fragment of claim 320, for use in treatment of cancer; and/or for use in treatment of infectious disease.

330) A pharmaceutical composition comprising at least one antibody or antigen-binding fragment thereof according to claim 320.

331) A vaccine composition comprising at least one antibody or antigen-binding fragment thereof according to claim 320 and an antigen.

332) The composition of claim 331 which is suitable for administration by a route selected from topical, or injection, and which optionally is suitable for administration by a route selected from intravascular delivery (e.g. injection or infusion), intravenous, intramuscular, intradermal, intraperitoneal, subcutaneous, spinal, enteral, rectal, pulmonary (e.g. inhalation), nasal, topical (including transdermal, buccal and sublingual), intravesical, intravitreal, intraperitoneal, vaginal, brain delivery (e.g. intra-cerebroventricular, intra-cerebral, and convection enhanced diffusion), CNS delivery (e.g. intrathecal, perispinal, and intra-spinal) or parenteral (including subcutaneous, intramuscular, intravenous and intradermal), transmucosal (e.g., sublingual administration), administration or administration via an implant, or other parenteral routes of administration, wherein “parenteral administration” refers to modes of administration other than enteral and topical administration; and/or which is suitable for administration by a route selected from, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

333) The composition of claim 330, which comprises at least one other active agent, e.g., a therapeutic or diagnostic agent, optionally selected from another immunomodulatory compound, a chemotherapeutic, a drug, a cytokine, a radionuclide, and an enzyme; and/or which comprises an antigen that is expressed by a target cell.

334) The composition of claim 333, which comprises or is used with another composition containing at least one immunomodulatory agent selected from PD-1 agonists and antagonists, PD-L1 and PD-L2 antibodies and antibody fragments, TLR agonists, CD40 agonists or antagonists, VISTA agonists or antagonists, CTLA-4 fusion proteins, CD28 agonists or antagonists, 4-1BB agonists or antagonists, CD27 or CD70 agonists or antagonists, LAG3 agonists or antagonists, TIM3 agonists or antagonists, TIGIT agonists or antagonists, ICOS agonists or antagonists, ICOS ligand agonists or antagonists.

335) A method of treatment comprising administering to a subject in need thereof at least one dosage or composition comprising a therapeutically effective amount of at least one anti-VSTM5 antibody, or antigen-binding fragment according to claim 320 or composition containing same.

336) The method of claim 335, wherein the disease is selected from the group consisting of cancer, autoimmune disease, or infectious disease.

337) The method of claim 336 which promotes anti-tumor immunity by suppressing one or more of the effects of VSTM5 on immunity.

338) The method of claim 336, which is used in the treatment of cancer, sepsis or an infectious condition or combination thereof.

339) The method of claim 336 further comprising administering another therapeutic agent useful for treating bacterial infection, viral infection, fungal infection, parasitic infection or sepsis.

340) The method of claim 336 which promotes an immune response against an infectious agent by suppressing one or more of the effects of VSTM5 on immunity.

341) The method of claim 339, wherein said agent is selected from the group consisting of antibiotics, drugs against mycobacteria; wherein said agent is selected from the group consisting of antiviral drugs; and/or wherein the agent is selected from the group consisting of antifungal drugs of the Polyene antifungals, Imidazole, triazole, and thiazole antifungals, Allylamines, Echinocandins or other anti-fungal drugs.

342) The method of claim 340, wherein the antibiotics are selected from the group consisting of Aminoglycosides, Carbapenems, Cephalosporins, Macrolides, Lincosamides, Nitrofurans, penicillins, Polypeptides, Quinolones, Sulfonamides, Tetracyclines; and/or wherein the drug against mycobacteria is selected from the group consisting of Clofazimine, Cycloserine, Cycloserine, Rifabutin, Rifapentine, Streptomycin and other antibacterial drugs such as Chloramphenicol, Fosfomycin, Metronidazole, Mupirocin, and Tinidazole, or a combination thereof; and/or antiviral drugs such as oseltamivir (brand name Tamiflu®) and zanamivir (brand name Relenza®) Arbidol® —adamantane derivatives (Amantadine®, Rimantadine®)—neuraminidase inhibitors (Oseltamivir®, Laninamivir®, Peramivir®, Zanamivir®) nucleotide analog reverse transcriptase inhibitor including Purine analogue guanine (Aciclovir®/Valacyclovir®, Ganciclovir®/Valganciclovir®, Penciclovir®/Famciclovir®) and adenine (Vidarabine®), Pyrimidine analogue, uridine (Idoxuridine®, Trifluridine®, Edoxudine®), thymine (Brivudine®), cytosine (Cytarabine®); Foscarnet; Nucleoside analogues/NARTIs: Entecavir, Lamivudine®, Telbivudine®, Clevudine®; Nucleotide analogues/NtRTIs: Adefovir®, Tenofovir; Nucleic acid inhibitors such as Cidofovir®; InterferonInterferon alfa-2b, Peginterferon α-2a; Ribavirin®/Taribavirin®; antiretroviral drugs including zidovudine, lamivudine, abacavir, lopinavir, ritonavir, tenofovir/emtricitabine, efavirenz each of them alone or a various combinations, gp41 (Enfuvirtide), Raltegravir®, protease inhibitors such as Fosamprenavir®, Lopinavir® and Atazanavir®, Methisazone®, Docosanol®, Fomivirsen®, and Tromantadine®.

343) The method of claim 336, wherein the treated individual suffers from cancer.

344) The method of claim 343, wherein the cancer is selected from the group consisting of breast cancer, cervical cancer, ovary cancer, endometrial cancer, melanoma, uveal melanoma, bladder cancer, lung cancer, pancreatic cancer, colorectal cancer, prostate cancer, hematological cancer, thyroid cancer, thyroid follicular cancer, myelodysplastic syndrome (MDS), fibrosarcomas and rhabdomyosarcomas, teratocarcinoma, neuroblastoma, glioma, glioblastoma, benign tumor of the skin, keratoacanthomas, renal cancer, esophageal cancer, follicular dendritic cell carcinoma, seminal vesicle tumor, epidermal carcinoma, spleen cancer, bladder cancer, head and neck cancer, stomach cancer, liver cancer, bone cancer, brain cancer, cancer of the retina, biliary cancer, small bowel cancer, salivary gland cancer, cancer of uterus, cancer of testicles, cancer of connective tissue, myelodysplasia, Waldenström's macroglobinaemia, nasopharyngeal, neuroendocrine cancer, mesothelioma, angiosarcoma, Kaposi's sarcoma, carcinoid, fallopian tube cancer, peritoneal cancer, papillary serous müllerian cancer, malignant ascites, gastrointestinal stromal tumor (GIST), Li-Fraumeni syndrome and Von Hippel-Lindau syndrome (VHL), cancer of unknown origin either primary or metastatic, wherein the cancer is non-metastatic, invasive or metastatic.

345) The method of claim 344, wherein the cancer is selected from B-cell lymphoma, Burkitt's lymphoma, leukemia, acute lymphocytic leukemia, chronic lymphocytic leukemia, B-cell lymphoma, Burkitt's lymphoma, multiple myeloma, Non-Hodgkin's lymphoma, myeloid leukemia, acute myelogenous leukemia (AML), chronic myelogenous leukemia, anaplastic large-cell lymphoma, esophageal squamous cells carcinoma, hepatocellular carcinoma cancer, follicular dendritic cell carcinoma, muscle-invasive cancer, seminal vesicle tumor, epidermal carcinoma, cancer of the retina, biliary cancer, small bowel cancer, salivary gland cancer, cancer of connective tissue, myelodysplasia, Waldenström's macroglobinaemia, nasopharyngeal, neuroendocrine cancer, myelodysplastic syndrome, mesothelioma, angiosarcoma, Kaposi's sarcoma, carcinoid, esophagogastric, fallopian tube cancer, peritoneal cancer, papillary serous müllerian cancer, malignant ascites, gastrointestinal stromal tumor (GIST), Li-Fraumeni syndrome and Von Hippel-Lindau syndrome (VHL); endometrial cancer, Breast carcinoma, optionally any of ductal-carcinoma, infiltrating ductal carcinoma, lobular carcinoma, mucinous adenocarcinoma, intra duct and invasive ductal carcinoma, and Scirrhous adenocarcinoma, Colorectal adenocarcinoma, preferably any of Poorly to Well Differentiated invasive and noninvasive Adenocarcinoma, Poorly to Well Differentiated Adenocarcinoma of the cecum, Well to Poorly Differentiated Adenocarcinoma of the colon, Tubular adenocarcinoma, preferably Grade 2 Tubular adenocarcinoma of the ascending colon, colon adenocarcinoma Duke's stage C1, invasive adenocarcinoma, Adenocarcinoma of the rectum, preferably Grade 3 Adenocarcinoma of the rectum, Moderately Differentiated Adenocarcinoma of the rectum, Moderately Differentiated Mucinous adenocarcinoma of the rectum; Lung cancer, preferably any of Well to Poorly differentiated Non-small cell carcinoma, Squamous Cell Carcinoma, preferably well to poorly Differentiated Squamous Cell Carcinoma, keratinizing squamous cell carcinoma, adenocarcinoma, preferably poorly to well differentiated adenocarcinoma, large cell adenocarcinoma, Small cell lung cancer, preferably Small cell lung carcinoma, more preferably undifferentiated Small cell lung carcinoma; Prostate adenocarcinoma, preferably any of Adenocarcinoma Gleason Grade 6 to 9, Infiltrating adenocarcinoma, High grade prostatic intraepithelial neoplasia, undifferentiated carcinoma; Stomach adenocarcinoma, preferably moderately differentiated gastric adenocarcinoma; Ovary carcinoma, preferably any of cystadenocarcinoma, serous papillary cystic carcinoma, Serous papillary cystic carcinoma, Invasive serous papillary carcinoma; Brain cancer, preferably any of Astrocytoma, with the proviso that it is not a grade 2 astrocytoma, preferably grade 4 Astrocytoma, Glioblastoma multiforme; Kidney carcinoma, preferably Clear cell renal cell carcinoma; Liver cancer, preferably any of Hepatocellular carcinoma, preferably Low Grade hepatocellular carcinoma, Fibrolamellar Hepatocellular Carcinoma; Lymphoma, preferably any of, Hodgkin's Lymphoma and High to low grade Non-Hodgkin's Lymphoma and with the proviso that if the cancer is brain cancer, it is not Astrocytoma grade 2, and if the cancer is Non-Hodgkin's Lymphoma, it is not a large cell Non-Hodgkin's Lymphoma, and wherein the cancer is non-metastatic, invasive or metastatic; and/or wherein said breast cancer is breast carcinoma, and is selected from the group consisting of ductal-carcinoma, infiltrating ductal carcinoma, lobular carcinoma, mucinous adenocarcinoma, intra duct and invasive ductal carcinoma, and Scirrhous adenocarcinoma; and/or wherein the cancer is a colon cancer selected from the group consisting of Poorly to Well Differentiated invasive and non-invasive Adenocarcinoma, Poorly to Well Differentiated Adenocarcinoma of the cecum, Well to Poorly Differentiated Adenocarcinoma of the colon, Tubular adenocarcinoma, preferably Grade 2 Tubular adenocarcinoma of the ascending colon, colon adenocarcinoma Duke's stage C1, invasive adenocarcinoma, Adenocarcinoma of the rectum, preferably Grade 3 Adenocarcinoma of the rectum, Moderately Differentiated Adenocarcinoma of the rectum, Moderately Differentiated Mucinous adenocarcinoma of the rectum; and/or wherein the cancer is a cancer is selected from the group consisting of Well to Poorly differentiated Non-small cell carcinoma, Squamous Cell Carcinoma, preferably well to poorly Differentiated Squamous Cell Carcinoma, keratinizing squamous cell carcinoma, adenocarcinoma, preferably poorly to well differentiated adenocarcinoma, large cell adenocarcinoma, Small cell lung cancer, preferably Small cell lung carcinoma, more preferably undifferentiated Small cell lung carcinoma; and/or wherein the cancer is a prostate adenocarcinoma selected from the group consisting of Adenocarcinoma Gleason Grade 6 to 9, Infiltrating adenocarcinoma, High grade prostatic intraepithelial neoplasia, undifferentiated carcinoma; and/or wherein the cancer is a stomach cancer comprising moderately differentiated gastric adenocarcinoma; and/or wherein the cancer is an ovarian cancer selected from the group consisting of cystadenocarcinoma, serous papillary cystic carcinoma, Serous papillary cystic carcinoma, Invasive serous papillary carcinoma; and/or wherein the cancer is a brain cancer selected from the group consisting Astrocytoma, with the proviso that it is not a grade 2 astrocytoma, preferably grade 4 Astrocytoma, and Glioblastoma multiforme; and/or wherein the cancer is clear cell renal cell carcinoma; and/or wherein the cancer is Hepatocellular carcinoma, optionally wherein the cancer is a Hepatocellular carcinoma selected from Low Grade hepatocellular carcinoma and Fibrolamellar Hepatocellular Carcinoma; and/or wherein the cancer is a lymphoma selected from the group consisting of Hodgkin's Lymphoma and High to low grade Non-Hodgkin's Lymphoma.

346) The method of claim 336 wherein the levels of VSTM5 protein are elevated compared to normal cell samples.

347) The method of claim 336, wherein the treated individual suffers from a cancer wherein the cancer or other cells contained at the tumor sites do not express VSTM5 protein at levels higher than normal.

348) The method of claim 336, wherein the treated subject suffers from a cancer wherein the diseased cells, APC's or other cells at the disease site express VSTM5 protein.

349) The method of claim 336 comprising administering an additional therapy which comprises one or more of radiotherapy, cryotherapy, antibody therapy, chemotherapy, photodynamic therapy, surgery, hormonal deprivation or combination therapy with conventional drugs.

350) The method of claim 336 further comprising administering another therapeutic agent selected from the group consisting of cytotoxic drugs, tumor vaccines, antibodies, peptides, pepti-bodies, small molecules, chemotherapeutic agents, cytotoxic and cytostatic agents, immunological modifiers, interferons, interleukins, immunostimulatory growth hormones, cytokines, vitamins, minerals, aromatase inhibitors, RNAi, Histone Deacetylase Inhibitors, and proteasome inhibitors.

351) The method of claim 336 further comprising administering one or more potentiating agents to obtain a therapeutic effect, wherein said one or more potentiating agents is selected from the group consisting of radiotherapy, conventional/classical anti-cancer therapy potentiating anti-tumor immune responses, Targeted therapy potentiating anti-tumor immune responses, Therapeutic agents targeting immunosuppressive cells Tregs and/or MDSCs, Immunostimulatory antibodies, Cytokine therapy, Adoptive cell transfer.

352) The method of claim 336, further comprising adoptive cell transfer therapy which is carried out following ex vivo treatment selected from expansion of the patient autologous naturally occurring tumor specific T cells or genetic modification of T cells to confer specificity for tumor antigens.

353) The method of claim 336 further comprising administering an conventional/classical anti-cancer agent selected from the group consisting of platinum based compounds, antibiotics with anti-cancer activity, Anthracyclines, Anthracenediones, alkylating agents, antimetabolites, Antimitotic agents, Taxanes, Taxoids, microtubule inhibitors, Vinca alkaloids, Folate antagonists, Topoisomerase inhibitors, Antiestrogens, Antiandrogens, Aromatase inhibitors, GnRh analogs, inhibitors of 5α-reductase, biphosphonates.

354) The method of claim 353, further comprising administering Platinum based compounds such as oxaliplatin, cisplatin, carboplatin; Antibiotics with anti-cancer activity, such as dactinomycin, bleomycin, mitomycin-C, mithramycin and Anthracyclines, such as doxorubicin, daunorubicin, epirubicin, idarubicin; Anthracenediones, such as mitoxantrone; Alkylating agents, such as dacarbazine, melphalan, cyclophosphamide, temozolomide, chlorambucil, busulphan, nitrogen mustard, nitrosoureas; Antimetabolites, such as fluorouracil, raltitrexed, gemcitabine, cytosine arabinoside, hydroxyurea and Folate antagonists, such as methotrexate, trimethoprim, pyrimethamine, pemetrexed; Antimitotic agents such as polokinase inhibitors and Microtubule inhibitors, such as Taxanes and Taxoids, such as paclitaxel, docetaxel; Vinca alkaloids such as vincristine, vinblastine, vindesine, vinorelbine; Topoisomerase inhibitors, such as etoposide, teniposide, amsacrine, topotecan, irinotecan, camptothecin; Cytostatic agents including Antiestrogens such as tamoxifen, fulvestrant, toremifene, raloxifene, droloxifene, iodoxyfene, Antiandrogens such as bicalutamide, flutamide, nilutamide and cyproterone acetate, Progestogens such as megestrol acetate, Aromatase inhibitors such as anastrozole, letrozole, vorozole, exemestane; GnRH analogs, such as leuprorelin, goserelin, buserelin, degarelix; inhibitors of 5α-reductase such as finasteride.

355) The method of claim 353, further comprising administering a targeted therapy selected from the group consisting of but not limited to: histone deacetylase (HDAC) inhibitors, such as vorinostat, romidepsin, panobinostat, belinostat, mocetinostat, abexinostat, entinostat, resminostat, givinostat, quisinostat, sodium butyrate; Proteasome inhibitors, such as bortezomib, carfilzomib, disulfiram; mTOR pathway inhibitors, such as temsirolimus, rapamycin, everolimus; PI3K inhibitors, such as perifosine, CAL101, PX-866, IPI-145, BAY 80-6946; B-raf inhibitors such as vemurafenib, sorafenib; JAK2 inhibitors, such as lestaurtinib, pacritinib; Tyrosine kinase inhibitors (TKIs), such as erlotinib, imatinib, sunitinib, lapatinib, gefitinib, sorafenib, nilotinib, toceranib, bosutinib, neratinib, vatalanib, regorafenib, cabozantinib; other Protein kinase inhibitors, such as crizotinib; Inhibitors of serine/threonine kinases for example Ras/Raf signalling inhibitors such as farnesyl transferase inhibitors; Inhibitors of serine proteases for example matriptase, hepsin, urokinase; Inhibitors of intracellular signaling such as tipifarnib, perifosine; Inhibitors of cell signalling through MEK and/or AKT kinases; aurora kinase inhibitors such as AZD1152, PH739358, VX-680, MLN8054, R763, MP235, MP529, VX-528, AX39459; Cyclin dependent kinase inhibitors such as CDK2 and/or CDK4 inhibitors; Inhibitors of survival signaling proteins including Bcl-2, Bcl-XL, such as ABT-737; HSP90 inhibitors; Therapeutic monoclonal antibodies, such as anti-EGFR mAbs cetuximab, panitumumab, nimotuzumab, anti-ERBB2 mAbs trastuzumab, pertuzumab, anti-CD20 mAbs such as rituximab, ofatumumab, veltuzumab and mAbs targeting other tumor antigens such as alemtuzumab, labetuzumab, adecatumumab, oregovomab, onartuzumab; TRAIL pathway agonists, such as dulanermin (soluble rhTRAIL), apomab, mapatumumab, lexatumumab, conatumumab, tigatuzumab; Antibody fragments, bi-specific antibodies and bi-specific T-cell engagers (BiTEs), such as catumaxomab, blinatumomab; Antibody drug conjugates (ADC) and other immunoconjugates, such as ibritumomab triuxetan, tositumomab, brentuximab vedotin, gemtuzumab ozogamicin, clivatuzumab tetraxetan, pemtumomab, trastuzumab emtansine; Anti-angiogenic therapy such as bevacizumab, etaracizumab, volociximab, ramucirumab, aflibercept, sorafenib, sunitinib, regorafenib, axitinib, nintedanib, motesanib, pazopanib, cediranib; Metalloproteinase inhibitors such as marimastat; Inhibitors of urokinase plasminogen activator receptor function; Inhibitors of cathepsin activity.

356) The method of claim 336, further comprising administering another antibody selected from cetuximab, panitumumab, nimotuzumab, trastuzumab, pertuzumab, rituximab, ofatumumab, veltuzumab, alemtuzumab, labetuzumab, adecatumumab, oregovomab, onartuzumab; apomab, mapatumumab, lexatumumab, conatumumab, tigatuzumab, catumaxomab, blinatumomab, ibritumomab triuxetan, tositumomab, brentuximab vedotin, gemtuzumab ozogamicin, clivatuzumab tetraxetan, pemtumomab, trastuzumab emtansine, bevacizumab, etaracizumab, volociximab, ramucirumab, aflibercept.

357) The method of claim 336, further comprising administering a Therapeutic cancer vaccine selected from exogenous cancer vaccines including proteins or peptides used to mount an immunogenic response to a tumor antigen, recombinant virus and bacteria vectors encoding tumor antigens, DNA-based vaccines encoding tumor antigens, proteins targeted to dendritic cell-based vaccines, whole tumor cell vaccines, gene modified tumor cells expressing GM-CSF, ICOS and/or Flt3-ligand, oncolytic virus vaccines.

358) The method of claim 336, further comprising administering a Cytokine therapy selected from one or more of the following cytokines such as IL-2, IL-7, IL-12, IL-15, IL-17, IL-18 and IL-21, IL-23, IL-27, GM-CSF, IFNα (interferon α), IFNα-2b, IFNβ, IFNγ, and their different strategies for delivery.


SEQUENCE LISTING (349 sequences)

SEQ ID NO: 1 - protein, 96 aa, Homo sapiens
TINATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 2 - protein, 119 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLH

SEQ ID NO: 3 - protein, 117 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYED

SEQ ID NO: 4 - DNA, 1520 bp, Homo sapiens
atgaggcctctgcccagcgggaggaggaagacccgaggcatctccctaggactcttcgccctctgcctggccgcagcccgctgtctgcagagtcagggtgtgtccctatacattcctcaggccaccatcaatgccactgtcaaagaagacatcctgctctcagttgagtactcctgtcatggagtgcccaccatcgaatggacatattcatccaattggggaacgcagaagatcgtggagtggaaaccagggactcaggccaacatctctcaaagccacaaggacagagtctgcacctttgacaacggctccatccagctcttcagcgtgggagtgagggattccggctactatgtcatcaccgtgacggagcgcctggggagcagccagtttggcaccatcgtgctgcacgtctctgagatcctctatgaagacctgcactttgtcgctgtcatccttgcttttctcgctgctgtggccgcagtattaatcagcctcatgtgggtttgtaataagtgtgcatataaatttcagaggaagagaagacacaaactcaaagaaagcacaactgaggagattgagctggaagatgttgagtgttagccaaggctgggcctgactgcattcctacctcaagaggaaaccattctccaacaaaaagagcaagcacagctattatacccattgtgtgtggtcctgttgcagcccgctcctaacaggacagtgggagattaacaacattgactgcatggagttgaggactgtggatgggacaaagctagtattaggactcgcgctaagttcaaggagaagagtgattgaggctttgaaccaggagcttcgcttggctgcagcatcagggccgtgctgacacataaccaatggcagtcggaggaaccaggctctgggccaggacagtttccagtgctgtgaagaaacgaggtgagcagtatccaaggggctcaaggataatctgctgatttctttctcgtctttcacacagagaggccaccagcaggaaagcagtgggagttgggtagctgctggggccagcatgtcctcttccacactacctgcctttcagaactctgctcttccgcttgtcccgaagtcagggctggcaatcttcataatcaaaggggagttggaaaagaacacccactaagaggcctccatggcagatgtcaattaaattgcccccccccccaaatctcatgacagtttattcacatcagtagtgtgggaagttacaatgttttttttaaaaaaagcttctttccttggctttccatttctttgaaaaaagggtttcttttgaatttttaaagctctgccatactgaacattcctgtggaaaggtttaaaatgcagagcctgaggtttttgcttttcagaaaaataaaaatcatagagattagctagtgtaaatgttaagctataaattatgtttcaaactgtgaaaaaaaaaagttttaaagaatgaatgaaataaaacctgaaaataaagccctggtcctttg

SEQ ID NO: 5 - DNA, 1650 bp, Homo sapiens
atgaggcctctgcccagcgggaggaggaagacccgaggcatctccctaggactcttcgccctctgcctggccgcagcccgctgtctgcagagtcagggtgtgtccctatacattcctcaggccaccatcaatgccactgtcaaagaagacatcctgctctcagttgagtactcctgtcatggagtgcccaccatcgaatggacatattcatccaattggggaacgcagaagatcgtggagtggaaaccagggactcaggccaacatctctcaaagccacaaggacagagtctgcacctttgacaacggctccatccagctcttcagcgtgggagtgagggattccggctactatgtcatcaccgtgacggagcgcctggggagcagccagtttggcaccatcgtgctgcacgtctctgagatcctctatgaagacctgcactttgtcgctgtcatccttgcttttctcgctgctgtggccgcagtattaatcagcctcatgtgggtttgtaataagtgtgcatataaatttcagaggaagagaagacacaaactcaaaggtaaccccctgggccttgtgataatccatgagtggttttgaggccagaggtggcatgtgttatcttgttctcacgaagaaagcactatgtggcttgatttccaactcattgacttgtctttgctttacagaaagcacaactgaggagattgagctggaagatgttgagtgttagccaaggctgggcctgactgcattcctacctcaagaggaaaccattctccaacaaaaagagcaagcacagctattatacccattgtgtgtggtcctgttgcagcccgctcctaacaggacagtgggagattaacaacattgactgcatggagttgaggactgtggatgggacaaagctagtattaggactcgcgctaagttcaaggagaagagtgattgaggctttgaaccaggagcttcgcttggctgcagcatcagggccgtgctgacacataaccaatggcagtcggaggaaccaggctctgggccaggacagtttccagtgctgtgaagaaacgaggtgagcagtatccaaggggctcaaggataatctgctgatttctttctcgtctttcacacagagaggccaccagcaggaaagcagtgggagttgggtagctgctggggccagcatgtcctcttccacactacctgcctttcagaactctgctcttccgcttgtcccgaagtcagggctggcaatcttcataatcaaaggggagttggaaaagaacacccactaagaggcctccatggcagatgtcaattaaattgcccccccccccaaatctcatgacagtttattcacatcagtagtgtgggaagttacaatgttttttttaaaaaaagcttctttccttggctttccatttctttgaaaaaagggtttcttttgaatttttaaagctctgccatactgaacattcctgtggaaaggtttaaaatgcagagcctgaggtttttgcttttcagaaaaataaaaatcatagagattagctagtgtaaatgttaagctataaattatgtttcaaactgtgaaaaaaaaaagttttaaagaatgaatgaaataaaacctgaaaataaagccctggtcctttg

SEQ ID NO: 6 - protein, 200 aa, Homo sapiens
MRPLPSGRRKTRGISLGLFALCLAAARCLQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLHFVAVILAFLAAVAAVLISLMWVCNKCAYKFQRKRRHKLKESTTEEIELEDVEC

SEQ ID NO: 7 - protein, 199 aa, Homo sapiens
MRPLPSGRRKTRGISLGLFALCLAAARCLQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLHFVAVILAFLAAVAAVLISLMWVCNKCAYKFQRKRRHKLKGNPLGLVIIHEWF

SEQ ID NO: 8 - protein, 352 aa, Artificial Sequence
  Fusion protein mouse VSTM5-ECD fused to mouse IgG2a Fc
LQSQGVSLYIPQSAINATVQQDILLSVDYICHGVPTIEWKYTPNWGVQRIVEWKPGTPANVSQSHRDRVCTFDNGSIQLFNVSVKDSGYYIVTVTEHPGSSQSGTILLRVSEIRYEDLHEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 9 - protein, 456 aa, Artificial Sequence [sequence omitted]

SEQ ID NO: 10 - protein, 389 aa, Artificial Sequence
  Fusion protein human VSTM5-ECD fused to mouse IgG2a Fc
MRPLPSGRRKTRGISLGLFALCLAAARCLQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDGSENLYFQGSGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 11 - protein, 199 aa, Homo sapiens
MRPLRCGERTQGIPLGLLAFWVTAARCLQSQGVSLYIPQSAINATVQQDILLSVDYICHGVPTIEWKYTPNWGVQRIVEWKPGTPANVSQSHRDRVCTFDNGSIQLFNVSVKDSGYYIVTVTEHPGSSQSGTILLRVSEIRYEDLHFVAVFFALLAAVAVVLISLMWVCNQCAYKFQRKRRYKLKESTTEEIEMKEVEC

SEQ ID NO: 12 - protein, 18 aa, Homo sapiens
TIEWTYSSNWGTQKIVEW

SEQ ID NO: 13 - protein, 19 aa, Homo sapiens
TIEWTYSSNWGTQKIVEWK

SEQ ID NO: 14 - protein, 19 aa, Homo sapiens
PTIEWTYSSNWGTQKIVEW

SEQ ID NO: 15 - protein, 20 aa, Homo sapiens
PTIEWTYSSNWGTQKIVEWK

SEQ ID NO: 16 - protein, 28 aa, Homo sapiens
GYYVITVTERLGSSQFGTIVLHVSEILY

SEQ ID NO: 17 - protein, 23 aa, Homo sapiens
GYYVITVTERLGSSQFGTIVLHV

SEQ ID NO: 18 - protein, 27 aa, Homo sapiens
GYYVITVTERLGSSQFGTIVLHVSEIL

SEQ ID NO: 19 - protein, 12 aa, Homo sapiens
RVCTFDNGSIQL

SEQ ID NO: 20 - protein, 17 aa, Homo sapiens
RVCTFDNGSIQLFSVGV

SEQ ID NO: 21 - protein, 12 aa, Homo sapiens
VCTFDNGSIQLF

SEQ ID NO: 22 - protein, 107 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIV

SEQ ID NO: 23 - protein, 106 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIV

SEQ ID NO: 24 - protein, 105 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIV

SEQ ID NO: 25 - protein, 108 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVL

SEQ ID NO: 26 - protein, 109 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 27 - protein, 110 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 28 - protein, 111 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 29 - protein, 112 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSE

SEQ ID NO: 30 - protein, 113 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEI

SEQ ID NO: 31 - protein, 114 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEIL

SEQ ID NO: 32 - protein, 115 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILY

SEQ ID NO: 33 - protein, 116 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYE

SEQ ID NO: 34 - protein, 118 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDL

SEQ ID NO: 35 - protein, 120 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLHF

SEQ ID NO: 36 - protein, 107 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVL

SEQ ID NO: 37 - protein, 108 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 38 - protein, 109 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 39 - protein, 110 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 40 - protein, 111 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSE

SEQ ID NO: 41 - protein, 112 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEI

SEQ ID NO: 42 - protein, 113 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEIL

SEQ ID NO: 43 - protein, 114 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILY

SEQ ID NO: 44 - protein, 115 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYE

SEQ ID NO: 45 - protein, 116 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYED

SEQ ID NO: 46 - protein, 117 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDL

SEQ ID NO: 47 - protein, 118 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLH

SEQ ID NO: 48 - protein, 119 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLHF

SEQ ID NO: 49 - protein, 106 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVL

SEQ ID NO: 50 - protein, 107 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 51 - protein, 108 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 52 - protein, 109 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 53 - protein, 110 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSE

SEQ ID NO: 54 - protein, 111 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEI

SEQ ID NO: 55 - protein, 112 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEIL

SEQ ID NO: 56 - protein, 113 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILY

SEQ ID NO: 57 - protein, 114 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYE

SEQ ID NO: 58 - protein, 115 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYED

SEQ ID NO: 59 - protein, 116 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDL

SEQ ID NO: 60 - protein, 117 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLH

SEQ ID NO: 61 - protein, 118 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLHF

SEQ ID NO: 62 - protein, 96 aa, Homo sapiens
QATINATVKEDILLSVEYSCHGVPTIEWTYSSNWGTQKIVEWKPGTQANISQSHKDRVCTFDNGSIQLFSVGVRDSGYYVITVTERLGSSQFGTIV

SEQ ID NO: 63 - protein, 97 aa, Homo sapiens
QATINATVKEDILLSVEYSCHGVPTIEWTYSSNWGTQKIVEWKPGTQANISQSHKDRVCTFDNGSIQLFSVGVRDSGYYVITVTERLGSSQFGTIVL

SEQ ID NO: 64 - protein, 98 aa, Homo sapiens
QATINATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 65 - protein, 99 aa, Homo sapiens
QATINATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 66 - protein, 100 aa, Homo sapiens
QATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 67 - protein, 95 aa, Homo sapiens
ATINATVKEDILLSVEYSCHGVPTIEWTYSSNWGTQKIVEWKPGTQANISQSHKDRVCTFDNGSIQLFSVGVRDSGYYVITVTERLGSSQFGTIV

SEQ ID NO: 68 - protein, 96 aa, Homo sapiens
ATINATVKEDILLSVEYSCHGVPTIEWTYSSNWGTQKIVEWKPGTQANISQSHKDRVCTFDNGSIQLFSVGVRDSGYYVITVTERLGSSQFGTIVL

SEQ ID NO: 69 - protein, 97 aa, Homo sapiens
ATINATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 70 - protein, 98 aa, Homo sapiens
ATINATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 71 - protein, 99 aa, Homo sapiens
ATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 72 - protein, 94 aa, Homo sapiens
TINATVKEDILLSVEYSCHGVPTIEWTYSSNWGTQKIVEWKPGTQANISQSHKDRVCTFDNGSIQLFSVGVRDSGYYVITVTERLGSSQFGTIV

SEQ ID NO: 73 - protein, 95 aa, Homo sapiens
TINATVKEDILLSVEYSCHGVPTIEWTYSSNWGTQKIVEWKPGTQANISQSHKDRVCTFDNGSIQLFSVGVRDSGYYVITVTERLGSSQFGTIVL

SEQ ID NO: 74 - protein, 97 aa, Homo sapiens
TINATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 75 - protein, 98 aa, Homo sapiens
TINATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 76 - protein, 93 aa, Homo sapiens
INATVKEDILLSVEYSCHGVPTIEWTYSSNWGTQKIVEWKPGTQANISQSHKDRVCTFDNGSIQLFSVGVRDSGYYVITVTERLGSSQFGTIV

SEQ ID NO: 77 - protein, 94 aa, Homo sapiens
INATVKEDILLSVEYSCHGVPTIEWTYSSNWGTQKIVEWKPGTQANISQSHKDRVCTFDNGSIQLFSVGVRDSGYYVITVTERLGSSQFGTIVL

SEQ ID NO: 78 - protein, 95 aa, Homo sapiens
INATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 79 - protein, 96 aa, Homo sapiens
INATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 80 - protein, 97 aa, Homo sapiens
INATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 81 - protein, 92 aa, Homo sapiens
NATVKEDILLSVEYSCHGVPTIEWTYSSNWGTQKIVEWKPGTQANISQSHKDRVCTFDNGSIQLFSVGVRDSGYYVITVTERLGSSQFGTIV

SEQ ID NO: 82 - protein, 93 aa, Homo sapiens
NATVKEDILLSVEYSCHGVPTIEWTYSSNWGTQKIVEWKPGTQANISQSHKDRVCTFDNGSIQLFSVGVRDSGYYVITVTERLGSSQFGTIVL

SEQ ID NO: 83 - protein, 94 aa, Homo sapiens
NATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 84 - protein, 95 aa, Homo sapiens
NATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 85 - protein, 96 aa, Homo sapiens
NATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 86 - protein, 103 aa, Homo sapiens
SLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 87 - protein, 104 aa, Homo sapiens
SLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 88 - protein, 105 aa, Homo sapiens
SLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 89 - protein, 106 aa, Homo sapiens
SLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSE

SEQ ID NO: 90 - protein, 107 aa, Homo sapiens
SLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEI

SEQ ID NO: 91 - protein, 102 aa, Homo sapiens
LYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 92 - protein, 103 aa, Homo sapiens
LYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 93 - protein, 104 aa, Homo sapiens
LYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 94 - protein, 105 aa, Homo sapiens
LYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSE

SEQ ID NO: 95 - protein, 106 aa, Homo sapiens
LYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEI

SEQ ID NO: 96 - protein, 101 aa, Homo sapiens
YIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 97 - protein, 102 aa, Homo sapiens
YIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 98 - protein, 103 aa, Homo sapiens
YIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 99 - protein, 104 aa, Homo sapiens
YIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSE

SEQ ID NO: 100 - protein, 105 aa, Homo sapiens
YIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEI

SEQ ID NO: 101 - protein, 100 aa, Homo sapiens
IPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 102 - protein, 101 aa, Homo sapiens
IPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 103 - protein, 102 aa, Homo sapiens
IPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 104 - protein, 103 aa, Homo sapiens
IPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSE

SEQ ID NO: 105 - protein, 104 aa, Homo sapiens
IPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEI

SEQ ID NO: 106 - protein, 99 aa, Homo sapiens
PQATINATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 107 - protein, 100 aa, Homo sapiens
PQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 108 - protein, 101 aa, Homo sapiens
PQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 109 - protein, 102 aa, Homo sapiens
PQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSE

SEQ ID NO: 110 - protein, 103 aa, Homo sapiens
PQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEI

SEQ ID NO: 111 - protein, 379 aa, Artificial Sequence
  Fusion protein human VSTM5-ECD fused to human IgG1 Fc C220S
MRPLPSGRRKTRGISLGLFALCLAAARCLQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLHEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 233 aa, Mus Musculus
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 113 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 232 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 217 aa, Mus Musculus
APNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 117 - protein, 2 aa, Artificial sequence
  Synthetic polypeptide linker
GS

SEQ ID NO: 118 - protein, 4 aa, Artificial sequence
  Synthetic polypeptide linker
GSGS

SEQ ID NO: 119 - protein, 2 aa, Artificial sequence
  Synthetic polypeptide linker
AS

SEQ ID NO: 120 - protein, 4 aa, Artificial sequence
  Synthetic polypeptide linker
GGGS

SEQ ID NO: 121 - protein, 5 aa, Artificial sequence
  Synthetic polypeptide linker
GGGGS

SEQ ID NO: 122 - protein, 10 aa, Artificial sequence
  Synthetic polypeptide linker
GGGGSGGGGS

SEQ ID NO: 123 - protein, 15 aa, Artificial sequence
  Synthetic polypeptide linker
GGGGSGGGGSGGGGS

SEQ ID NO: 124 - protein, 20 aa, Artificial sequence
  Synthetic polypeptide linker
GGGGSGGGGSGGGGSGGGGS

SEQ ID NO: 125 - protein, 12 aa, Artificial sequence
  Synthetic polypeptide linker
AGAAAKGAAAKA

SEQ ID NO: 126 - protein, 17 aa, Artificial sequence
  Synthetic polypeptide linker
AGAAAKGAAAKGAAAKA

SEQ ID NO: 127 - protein, 22 aa, Artificial sequence
  Synthetic polypeptide linker
AGAAAKGAAAKGAAAKGAAAKA

SEQ ID NO: 128 - protein, 27 aa, Artificial sequence
  Synthetic polypeptide linker
AGAAAKGAAAKGAAAKGAAAKGAAAKA

SEQ ID NO: 129 - protein, 7 aa, Artificial sequence
  Synthetic polypeptide linker
AGAAAKA

SEQ ID NO: 130 - protein, 351 aa, Artificial Sequence
  Fusion protein human VSTM5-ECD fused to human IgG1 Fc
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLHEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 361 aa, Artificial Sequence
  Fusion protein human VSTM5-ECD fused to mouse IgG2a Fc
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDGSENLYFQGSGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 132 - protein, 172 aa, Homo Sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLHFVAVILAFLAAVAAVLISLMWVCNKCAYKFQRKRRHKLKESTTEEIELEDVEC

SEQ ID NO: 133 - protein, 428 aa, Artificial Sequence
  Fusion protein human VSTM5-ECD fused to EGFP
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLHFVAVILAFLAAVAAVLISLMWVCNKCAYKFQRKRRHKLKESTTEEIELEDVECRILQSTVPRARDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 134 - protein, 337 aa, Artificial Sequence
  Fusion protein human VSTM5-ECD fused to human IgG1 Fc C220S
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 347 aa, Artificial Sequence
  Fusion protein human VSTM5-ECD fused to human IgG1 Fc C220S (G4S)x2 linker
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 338 aa, Artificial Sequence
  Fusion protein mouse VSTM5-ECD fused to mouse IgG2a Fc
LQSQGVSLYIPQSAINATVQQDILLSVDYICHGVPTIEWKYTPNWGVQRIVEWKPGTPANVSQSHRDRVCTFDNGSIQLFNVSVKDSGYYIVTVTEHPGSSQSGTEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 137 - protein, 348 aa, Artificial Sequence
  Fusion protein mouse VSTM5-ECD fused to mouse IgG2a Fc (G4S)x2 linker
LQSQGVSLYIPQSAINATVQQDILLSVDYICHGVPTIEWKYTPNWGVQRIVEWKPGTPANVSQSHRDRVCTFDNGSIQLFNVSVKDSGYYIVTVTEHPGSSQSGTGGGGSGGGGSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 138 - protein, 11 aa, Artificial Sequence
  synthetic polypeptide linker
GSENLYFQGSG

SEQ ID NO: 139 - protein, 25 aa, Artificial Sequence
  synthetic polypeptide linker
AEAAAKEAAAKAHELICALLINKER

SEQ ID NO: 140 - protein, 22 aa, Artificial Sequence
  synthetic polypeptide linker
AEAAAKEAAAKEAAAKEAAAKA

SEQ ID NO: 141 - protein, 14 aa, Artificial Sequence
  synthetic polypeptide linker
GGEAAAKEAAAKGG

SEQ ID NO: 142 - protein, 19 aa, Artificial Sequence
  synthetic polypeptide linker
GGGGSGGGGSGAGSGGGGS

SEQ ID NO: 143 - protein, 11 aa, Artificial Sequence
  synthetic polypeptide linker
GGGGGSGGGGS

SEQ ID NO: 144 - protein, 6 aa, Artificial Sequence
  synthetic polypeptide linker
GGGGSG

SEQ ID NO: 145 - protein, 4 aa, Artificial Sequence
  synthetic polypeptide linker
GSGG

SEQ ID NO: 146 - protein, 5 aa, Artificial Sequence
  synthetic polypeptide linker
GSGGS

SEQ ID NO: 147 - protein, 352 aa, Artificial Sequence
  Fusion protein mouse VSTM5-ECD fused to mouse IgG2a Fc N297A
LQSQGVSLYIPQSAINATVQQDILLSVDYICHGVPTIEWKYTPNWGVQRIVEWKPGTPANVSQSHRDRVCTFDNGSIQLFNVSVKDSGYYIVTVTEHPGSSQSGTILLRVSEIRYEDLHEPRGPTIKPCPPCKCPAPNLLGGPSVFIFPPKIKDVLMISLSPIVTCVVVDVSEDDPDVQISWFVNNVEVHTAQTQTHREDYASTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 148 - protein, 233 aa, Artificial Sequence
  Fusion protein mouse VSTM5-ECD fused to mouse IgG2a Fc N297A
EPRGPTIKPCPPCKCPAPNLLGGPSVFIFPPKIKDVLMISLSPIVTCVVVDVSEDDPDVQISWFVNNVEVHTAQTQTHREDYASTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 149 - protein, 351 aa, Artificial Sequence
  Fusion protein human VSTM5-ECD fused to human IgG1 Fc C220S N297A
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLHEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 232 aa, Artificial Sequence
  human IgG1 Fc C220S N297A
EPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 105 aa, Homo Sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGT

SEQ ID NO: 152 - protein, 104 aa, Homo Sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGT

SEQ ID NO: 153 - protein, 103 aa, Homo Sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGT

SEQ ID NO: 154 - protein, 106 aa, Homo Sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTI

SEQ ID NO: 155 - protein, 105 aa, Homo Sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTI

SEQ ID NO: 156 - protein, 104 aa, Homo Sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTI

SEQ ID NO: 157 - DNA, 363 bp, Artificial Sequence
  synthetic polynucleotide antibody heavy chain
gaggtgcagctggtggagtctgggggaggcttggtccagcctgggaggtccctgagactctcctgtgcagcctctggattcaccttcagtagctatgctatgcactgggtccgccaggctccaggtaaggggctggagtgggtggcagttatatcatatgatggaagtaataaatactacgcagactccgtgaagggccgattcaccatctccagagacaattccaagaacacgctgtatctgcaaatgaacagcctgagagctgaggacacggctgtgtattactgtgcgaaagatcagtattccgtgggagctactacttatgactactggggccagggaaccctggtcaccgtctcttca

SEQ ID NO: 158 - DNA, 336 bp, Artificial Sequence
  synthetic polynucleotide antibody light chain
cagcctgtgctgactcaatcatcctctgcctctgcttccctgggagcctcggtcaagctcacctgcagtctgagcagtgggcacagcagctacgccatcgcatggcatcagcagcagccagagaagggccctcgatacttgatgaaacttaacagtgatggcagccacagcaagggggacgggatccctgatcgcttctcaggctccagctctggggctgagcgctacctcaccatctccagcctccagtctgaggatgaggctgactattactgtcagacctggggctcaggcattcaggtgttcggcggagggaccaagctgaccgtcctaggt

SEQ ID NO: 159 - DNA, 354 bp, Artificial Sequence
  synthetic polynucleotide antibody heavy chain
gaggtgcagctggtggagtctgggggaggcttggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgagtaagagggagctacattcctttgactactggggccagggaaccctggtcaccgtctcctca

SEQ ID NO: 160 - DNA, 339 bp, Artificial Sequence
  synthetic polynucleotide antibody light chain
gatgttgtgatgactcagtctccactctccctacccgtcacccctggagagccggcctccatctcctgcaggtctagtcagagcctcctacaaagtaatggacacaactatttgaattggtacctgcagaagccagggcagtctccacaggtcctgatctatttggcttctaatcgggcctccggggtccctgacaggttcagtggcagtggatcaggcacagattttacactgaaaatcagcagagtggaggctgaggatgttgggatttattactgcatgcaaggtctacaaattcctctcactttcggcggagggaccaaggtggaaatcaaacgt

SEQ ID NO: 161 - DNA, 366 bp, Artificial Sequence
  synthetic polynucleotide antibody heavy chain
cagatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcactcatttattgggatgatgataagcgctacagcccatctctgaagagcaggctcaccatcaccaaggacacctccaaaaaccaggtggtccttacaatgaccaacatggaccctgtggacacagccacatattactgtgcacacagtggttctgtcggttacgctctctactttgactactggggccagggaaccctggtcaccgtctcctca

SEQ ID NO: 162 - DNA, 351 bp, Artificial Sequence
  synthetic polynucleotide antibody light chain
caggctgtgctgactcagccaccttcctcctccgcatctcctggagaatccgccagactcacctgcaccttgcccagtgacatcaatgttcgttactacaacatatactggtaccagcagaagccagggagccctcccaggtatctcctgtattaccagtcagactcacataagggccggggctctggagtccccagccgcttctctggatccaaagatacttcagccaatacagggattttactcatctccgggctccagtctgaggatgaggctgactattactgtatgatttgggcaagcaatggttctggggtgctcggcggaggcacccagctgaccgtcctaggt

SEQ ID NO: 163 - DNA, 378 bp, Artificial Sequence
  synthetic polynucleotide antibody heavy chain
caggtccagcttgtgcagtctggggctgaggtgaagaagcctgggacctcagtgaaggtttcctgcaaggcttctggatacaccttcactacctatactatgcattgggtgcgccaggcccccggacaaaggcttgagtggatgggatggatcaacactggcaatggtaacacaaaatattcacagaagttccaggacagagtcaccattaccagggacacatccgcgagcacagcctacatggagctgagcagcctgaaatttgaagacacggctgtatattactgtgcgagagagggggttacgatttttggagaccactcctactactacggtatggacgtctggggccaagggaccacggtcaccgtctcctca

SEQ ID NO: 164 - DNA, 336 bp, Artificial Sequence
  synthetic polynucleotide antibody light chain
cagcctgtgctgactcaatcatcctctgcctctgcttccctgggatcctcggtcaagctcacctgcactctgagcagtgggcacagtagctacatcatcgcatggcatcagcagcagccagggaaggcccctcgatacttgatgaagcttgaaggtagtggaagctacaacaaggggagcggacttcctgatcgcttctcaggctccagctctggggctgaccgctacctcaccatctccaacctccagtctgaggatgaggctgattattactgtgagacctgggacggtaacacttgggtgttcggcggaggcacccagctgaccgtcctaggt

SEQ ID NO: 165 - DNA, 369 bp, Artificial Sequence
  synthetic polynucleotide antibody heavy chain
gaggtgcagctggtggagtctgggggaggtgtggtcaagccgggggagtccctgagactcttatgtacagcctctggattgtccttcaataaatacagcataaattgggtccgccaggctccagggggggggcttgagtgggtctcatcgattgaaagtggtagtggacatatatattacgcagactcactggagggccgcttcaccatctccagagataacgccaagaactccgtgactctggaaatgaacagcctgagagtcgaggacacggctctttattactgtgtctcggggccggaagacaagtggttgttgcagctttactttgagtcctggggccagggaaccctggtcaccgtctcctca

SEQ ID NO: 166 - DNA, 327 bp, Artificial Sequence
  synthetic polynucleotide antibody light chain
gaaattgtgttgacgcagtctccagccaccctgtctttgtctccaggggaaagagccaccctctcctgcaggaccagtcagagttctcccagcgacaacttagcctggtatcagcacaaacctggccaggctccgaggctcctcatctacggtggttccaacagggccactggcatcccagccaggttcagtggcagtgggtctgggacagacttcactctcaccatcagcagcctagagcctgaagattttgcagtgtattactgtcagcagtatggtagctcaccgctcactttcggcggagggaccaaagtggatatcaaacgt

SEQ ID NO: 167 - DNA, 354 bp, Artificial Sequence
  synthetic polynucleotide antibody heavy chain
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagaggctggcgaggtggtagctttatggacgtctggggccaagggaccacggtcaccgtctcctca

SEQ ID NO: 168 - DNA, 330 bp, Artificial Sequence
  synthetic polynucleotide antibody light chain
cagtctgccctgactcagcctcgctcagtgtccgggtctcctggacagtcagtcaccatctcctgcactggaaccagcagtgatgttggtggttataactatgtctcctggtaccaacagcacccaggcaaagcccccaaactcatgatttatgatgtcattaagcggccctcaggggtccctgatcgcttctctggctccaggtctggcaacacggcctccctgaccatctctgggctccaggctgaagatgaggctgattattattgctgctcatatgcaggcacccattgggtgttcggcggagggaccaagctcaccgtcctaggt

SEQ ID NO: 169 - DNA, 363 bp, Artificial Sequence
  synthetic polynucleotide antibody heavy chain
gaggtgcagctggtggagtctgggggaggcttggtccagcctggggggtccctgagactctcctgtgcagcctctggattcacctttagcagctatgccatgacctgggtccgccaggctccagggaaggggctggagtgggtctcagttattagtggtggtggtggtaccacatactacgcagactccgtgaagggccgcttcaccatctccagagacaattccaagaacactctgtatctgcaaatgagcagcctgagagccgaggacacggccgtatattactgtgcgaaagggtatagcagtggctggccctactactttgactactggggccagggaaccctggtcaccgtctcctca

SEQ ID NO: 170 - DNA, 327 bp, Artificial Sequence
  synthetic polynucleotide antibody light chain
gaaattgtgttgacgcagtctccaggcaccctgtctttgtctccaggggaaagagccaccctctcctgcagggccagtcagagtggcgtcaccaatttcttagcctggtatcagcagaaacctggccaggctcccaggctcctcatctatgctacttccagcagggccactggcatcccagacaggttcagtggcagtgcgtctgggacagacttcactctcaccatcaccagactggagcctgaagattttgcagtttatttctgccagcaatatgcttcctcaccgctcactttcggcggagggaccaaagtggagatcaaacgt

SEQ ID NO: 171 - DNA, 366 bp, Artificial Sequence
  synthetic polynucleotide antibody heavy chain
caggtgcagctggtgcaatctggagcagaggtgaaaaagcccggggagtctctgaagatctcctgtaagggttctggatacgactttaataattactggatcggctgggtgcgccagacgcccgagaagggcctggagtggatggggatcgtctatcctggtgaccatcctggtgactatcatatcagatatggcccgtccttccaaggccaggtcaccatctcagccgacaggtccatcaccaccgcctacctacagtggagaaacctgaaggcctcggacaccgccatgtattactgtgcgagactaggaagcagtaaagaccttgactactggggccagggaaccctggtcaccgtctcctca

SEQ ID NO: 172 - DNA, 324 bp, Artificial Sequence
  synthetic polynucleotide antibody light chain
gaaattgtgttgacgcagtctccagccaccctgtctttgtctccaggggaaagagccaccctctcctgcagggccagtcagagtgttagcggacacttagactggtaccaacagaaacctggccagtctcccaggctcctcatctatgatgcatccaacagggccactggcatcccagccaggttcagtggcagtgggtctgggacagacttctctctcaccatcagcagcctagagcctgaagattttgcagtttattactgtcagcaccgtagcaactggccgtggacgttcggccaagggaccaaggtggaaatcaaacgt

SEQ ID NO: 173 - DNA, 357 bp, Artificial Sequence
  synthetic polynucleotide antibody heavy chain
caggtgcagctgcaggagtcgggcccaggactgatgaagccttcacagaccctgtccctcacctgcactgtctctggtgactccatcaccagaggtagcagttactggagttggatccggcagtccgacgggaagggactggagtggattgggcacatctatagtggaggggacaccgactacaatcccgccctcaagagtcgagtcactatatcagctgacgcgtccaggggccagtttttgttgagattgacctcaatgaccgccgcagacacggccgtttattactgtgcgagagatcgtggagcatacggtatggacgtctggggccaagggaccacggtcaccgtctcctca

SEQ ID NO: 174 - DNA, 336 bp, Artificial Sequence
  synthetic polynucleotide antibody light chain
cagcctgtgctgactcaatcatcctctgcctctgcttccctgggagcctcggtcaagctcacctgcactctgagcagtgaacacgacagatatgccatcgcatggcttcaacagaagccagagaagggtcctcgctacttgatgaaggttaacagtgatggcagccacaggaagggggacgggatccctgatcgcttctcaggctccagttctggggctgagcgctacctcaccatctccagactccagtctgaggatgaggctgactattactgtcagacctggggcattggcattagggtgttcggcggtggcacccagctgaccgtcctaggt

SEQ ID NO: 175 - DNA, 378 bp, Artificial Sequence
  synthetic polynucleotide antibody heavy chain
caggtgcagctggtggagtctgggggaggcgtggtccagcctgggaggtcccgcagactctcctgtgcagcgtctggattcgccttccgtaattatggcatgcactgggtccgccaggctccaggcaaggggctggagtgggtggcattcatatcacaaaacggaggtaagaaatattatgcagactccgtgacgggccgattcaccgtctccagagacaattccaagaacacgttgtatctgcaaataaacagcctgacaactgacgacacggctgtgttttactgtgcgaggtcggggagcgggtcatggggctacagtgacttccccggaccctttgaccactggggccagggatccctggtcactgtctcctca

SEQ ID NO: 176 - DNA, 327 bp, Artificial Sequence
  synthetic polynucleotide antibody light chain
gaaattgtgttgacgcagtctccaggcaccctgtctttgtctccaggggaaagagccaccctctcctgcagggccagtcagaatattttcatcagcttcttagcctggtaccagcagaaacctggccaggctcccaggctcctcctctatggtgcttccaacagggccactggcatcccagacaggttcagtggcagtgggtctgggacagacttcactctcaccatcagcagactggagcctgaagattttgcagtgtattactgtcagcagtatggtagctccccgctcactttcggcggagggaccaaagtggagatcaaacgt

SEQ ID NO: 177 - DNA, 360 bp, Artificial Sequence
  synthetic polynucleotide antibody heavy chain
caggtgcagctggtggagtctgggggaggcgtggtccagcctggggggtccttgaaactctcctgtgtagcctctggattcacctttagcgcctatgccatgaactgggtccgcctggttccaggtaaggggctggagtgggtctcaggtattagtggcaatggctattccacattctacccagactccgtgcagggccgattcaccgtctccagagacaattccaagaacacgttgtttctgcaaattgataggctgacaggcggggacacggccatatactactgtgcgaaggtacagactacggttattactccttttcaaaactggggccagggaaccctggtcaccgtctcttca

SEQ ID NO: 178 - DNA, 327 bp, Artificial Sequence
  synthetic polynucleotide antibody light chain
gaaattgtgttgacgcagtctccaggcaccctgtctttgtctccaggggaaagagccaccctctcctgcagggccagtcagaatattggcagcaacttcttagcctggtaccagcaaaaacctggccaggctcccaggctcctcatctatggtgcgtccaccagggccaatggcatcccagacaggttcagtggcagtaagtctgagacagacttcactctcaccatcagcagactggagcctgaagattttgcagtgtattactgtcagcagtatgataactcaccgtacacttttggccaggggaccaagctggagatcaaacgt

SEQ ID NO: 179 - DNA, 357 bp, Artificial Sequence
  synthetic polynucleotide antibody heavy chain
gaggtgcagctggtggagattggaggaggcttgatccagcctgggaggtccctgagactctcctgtgcagcctctggattcaccttcagtagctatgctatgcactgggtccgccaggctccaggcaaggggctggagtgggtggcggttatatcatatgatggaagcaaaaaatactacgcagactccgtgaagggccgattcaccatctccagagacaattccaagaacacggtgcatctgcaaatgaacagcctgagagtcgaggatacggctgtctattactgtgcgctcttgtcccgtccacactacggtttggacgtctggggccaagggaccacggtcaccgtctcctca

SEQ ID NO: 180 - DNA, 333 bp, Artificial Sequence
  synthetic polynucleotide antibody light chain
cagtctgtgctgacgcagccgccctcagtgtctgcggccccaggacagaaggtcaccatctcctgctctggaagtcgctccaacgttgggggtaattttctttcctggtaccaacacgtcccaggaacacccccccaactcctcatttatgacaattataagcgaccctcagagatacctgaccgattctcgggctccaagtctggcacgtcagccaccctggacatcaccggactccagactggggacgaggccgattattactgcggaacatgggatagcagcctgagttcttgggtgttcggcggaggcacccagctgaccgtcctaggt

SEQ ID NO: 181 - DNA, 24 bp, Artificial Sequence
  synthetic polynucleotide antibody CDR
ggattcaccttcagtagctatgct

SEQ ID NO: 182 - DNA, 24 bp, Artificial Sequence
  synthetic polynucleotide antibody CDR
atatcatatgatggaagtaataaa

SEQ ID NO: 183 - DNA, 42 bp, Artificial Sequence
  synthetic polynucleotide antibody CDR
gcgaaagatcagtattccgtgggagctactacttatgactac

SEQ ID NO: 184 - DNA, 21 bp, Artificial Sequence
  synthetic polynucleotide antibody CDR
agtgggcacagcagctacgcc

SEQ ID NO: 185 - DNA, 21 bp, Artificial Sequence
  synthetic polynucleotide antibody CDR
cttaacagtgatggcagccac

SEQ ID NO: 186 - DNA, 27 bp, Artificial Sequence
  synthetic polynucleotide antibody CDR
cagacctggggctcaggcattcaggtg

SEQ ID NO: 187 - DNA, 24 bp, Artificial Sequence
  synthetic polynucleotide antibody CDR
ggattcaccttcagtagctatgct

SEQ ID NO: 188 - DNA, 24 bp, Artificial Sequence
  synthetic polynucleotide antibody CDR
atatcatatgatggaagcaataaa

SEQ ID NO: 189 - DNA, 33 bp, Artificial Sequence
  synthetic polynucleotide antibody CDR
gcgagtaagagggagctacattcctttgactac

SEQ ID NO: 190 - DNA, 33 bp, Artificial Sequence
  synthetic polynucleotide antibody CDR
cagagcctcctacaaagtaatggacacaactat

SEQ ID NO: 191 - DNA, 9 bp, Artificial Sequence
  synthetic polynucleotide antibody CDR
ttggcttct

SEQ ID NO: 192 - DNA, 27 bp, Artificial Sequence
  synthetic polynucleotide antibody CDR
atgcaaggtctacaaattcctctcact

SEQ ID NO: 193 - DNA, 30 bp, Artificial Sequence
  synthetic polynucleotide antibody CDR
gggttctcactcagcactagtggagtgggt

SEQ ID NO: 194 - DNA, 21 bp, Artificial Sequence
  synthetic polynucleotide antibody CDR
atttattgggatgatgataag

SEQ ID NO: 195 - DNA, 42 bp, Artificial Sequence
  synthetic polynucleotide antibody CDR
gcacacagtggttctgtcggttacgctctctactttgactac

SEQ ID NO: 196 - DNA, 27 bp, Artificial Sequence
  synthetic polynucleotide antibody CDR
agtgacatcaatgttcgttactacaac

SEQ ID NO: 197 - DNA, 21 bp, Artificial Sequence
  synthetic polynucleotide antibody CDR
taccagtcagactcacataag

SEQ ID NO: 198 - DNA, 30 bp, Artificial Sequence
  synthetic polynucleotide antibody CDR
atgatttgggcaagcaatggttctggggtg

SEQ ID NO: 199 - DNA, 24 bp, Artificial Sequence
  synthetic polynucleotide antibody CDR
ggatacaccttcactacctatact

SEQ ID NO: 200 - DNA, 24 bp, Artificial Sequence
  synthetic polynucleotide antibody CDR
atcaacactggcaatggtaacaca

SEQ ID NO: 201 - DNA, 57 bp, Artificial Sequence
  synthetic polynucleotide antibody CDR
gcgagagagggggttacgatttttggagaccactcctactactacggtatggacgtc

SEQ ID NO: 202 - DNA, 21 bp, Artificial Sequence
  synthetic polynucleotide antibody CDR
agtgggcacagtagctacatc

SEQ ID NO: 203 - DNA, 21 bp, Artificial Sequence
  synthetic polynucleotide antibody CDR
cttgaaggtagtggaagctac

SEQ ID NO: 204 - DNA, 27 bp, Artificial Sequence
  synthetic polynucleotide antibody CDR
gagacctgggacggtaacacttgggtg

SEQ ID NO: 205 - DNA, 24 bp, Artificial Sequence
  synthetic polynucleotide antibody CDR
ggattgtccttcaataaatacagc

SEQ ID NO: 206 - DNA, 24 bp, Artificial Sequence
  synthetic polynucleotide antibody CDR
attgaaagtggtagtggacatata

SEQ ID NO: 207 - DNA, 48 bp, Artificial Sequence
  synthetic polynucleotide antibody CDR
gtctcggggccggaagacaagtggttgttgcagctttactttgagtcc

SEQ ID NO: 208 - DNA, 21 bp, Artificial Sequence
  synthetic polynucleotide antibody CDR
cagagttctcccagcgacaac

SEQ ID NO: 209 - DNA, 9 bp, Artificial Sequence
  synthetic polynucleotide antibody CDR
ggtggttcc

SEQ ID NO: 210 - DNA, 27 bp, Artificial Sequence
  synthetic polynucleotide antibody CDR
cagcagtatggtagctcaccgctcact

SEQ ID NO: 211 - DNA, 24 bp, Artificial Sequence
  synthetic polynucleotide antibody CDR
ggtgggtccttcagtggttactac

SEQ ID NO: 212 - DNA, 21 bp, Artificial Sequence
  synthetic polynucleotide antibody CDR
atcaatcatagtggaagcacc

SEQ ID NO: 213 - DNA, 36 bp, Artificial Sequence
  synthetic polynucleotide antibody CDR
gcgagaggctggcgaggtggtagctttatggacgtc

SEQ ID NO: 214 - DNA, 27 bp, Artificial Sequence
  synthetic polynucleotide antibody CDR
agcagtgatgttggtggttataactat

SEQ ID NO: 215 - DNA, 9 bp, Artificial Sequence
  synthetic polynucleotide antibody CDR
gatgtcatt

SEQ ID NO: 216 - DNA, 27 bp, Artificial Sequence
  synthetic polynucleotide antibody CDR
tgctcatatgcaggcacccattgggtg

SEQ ID NO: 217 - DNA, 24 bp, Artificial Sequence
  synthetic polynucleotide antibody CDR
ggattcacctttagcagctatgcc

SEQ ID NO: 218 - DNA, 24 bp, Artificial Sequence
  synthetic polynucleotide antibody CDR
attagtggtggtggtggtaccaca

SEQ ID NO: 219 - DNA, 42 bp, Artificial Sequence
  synthetic polynucleotide antibody CDR
gcgaaagggtatagcagtggctggccctactactttgactac

SEQ ID NO: 220 - DNA, 21 bp, Artificial Sequence
  synthetic polynucleotide antibody CDR
cagagtggcgtcaccaatttc

SEQ ID NO: 221 - DNA, 9 bp, Artificial Sequence
  synthetic polynucleotide antibody CDR
gctacttcc

SEQ ID NO: 222 - DNA, 27 bp, Artificial Sequence
  synthetic polynucleotide antibody CDR
cagcaatatgcttcctcaccgctcact

SEQ ID NO: 223 - DNA, 24 bp, Artificial Sequence
  synthetic polynucleotide antibody CDR
ggatacgactttaataattactgg

SEQ ID NO: 224 - DNA, 24 bp, Artificial Sequence
  synthetic polynucleotide antibody CDR
gtctatcctggtgaccatcctggt

SEQ ID NO: 225 - DNA, 45 bp, Artificial Sequence
  synthetic polynucleotide antibody CDR
atgtattactgtgcgagactaggaagcagtaaagaccttgactac

SEQ ID NO: 226 - DNA, 18 bp, Artificial Sequence
  synthetic polynucleotide antibody CDR
cagagtgttagcggacac

SEQ ID NO: 227 - DNA, 9 bp, Artificial Sequence
  synthetic polynucleotide antibody CDR
gatgcatcc

SEQ ID NO: 228 - DNA, 27 bp, Artificial Sequence
  synthetic polynucleotide antibody CDR
cagcaccgtagcaactggccgtggacg

SEQ ID NO: 229 - DNA, 30 bp, Artificial Sequence
  synthetic polynucleotide antibody CDR
ggtgactccatcaccagaggtagcagttac

SEQ ID NO: 230 - DNA, 21 bp, Artificial Sequence
  synthetic polynucleotide antibody CDR
atctatagtggaggggacacc

SEQ ID NO: 231 - DNA, 33 bp, Artificial Sequence
  synthetic polynucleotide antibody CDR
gcgagagatcgtggagcatacggtatggacgtc

SEQ ID NO: 232 - DNA, 21 bp, Artificial Sequence
  synthetic polynucleotide antibody CDR
agtgaacacgacagatatgcc

SEQ ID NO: 233 - DNA, 21 bp, Artificial Sequence
  synthetic polynucleotide antibody CDR
gttaacagtgatggcagccac

SEQ ID NO: 234 - DNA, 27 bp, Artificial Sequence
  synthetic polynucleotide antibody CDR
cagacctggggcattggcattagggtg

SEQ ID NO: 235 - DNA, 24 bp, Artificial Sequence
  synthetic polynucleotide antibody CDR
ggattcgccttccgtaattatggc

SEQ ID NO: 236 - DNA, 24 bp, Artificial Sequence
  synthetic polynucleotide antibody CDR
atatcacaaaacggaggtaagaaa

SEQ ID NO: 237 - DNA, 57 bp, Artificial Sequence
  synthetic polynucleotide antibody CDR
gcgaggtcggggagcgggtcatggggctacagtgacttccccggaccctttgaccac

SEQ ID NO: 238 - DNA, 21 bp, Artificial Sequence
  synthetic polynucleotide antibody CDR
cagaatattttcatcagcttc

SEQ ID NO: 239 - DNA, 9 bp, Artificial Sequence
  synthetic polynucleotide antibody CDR
ggtgcttcc

SEQ ID NO: 240 - DNA, 27 bp, Artificial Sequence
  synthetic polynucleotide antibody CDR
cagcagtatggtagctccccgctcact

SEQ ID NO: 241 - DNA, 24 bp, Artificial Sequence
  synthetic polynucleotide antibody CDR
ggattcacctttagcgcctatgcc

SEQ ID NO: 242 - DNA, 24 bp, Artificial Sequence
  synthetic polynucleotide antibody CDR
attagtggcaatggctattccaca

SEQ ID NO: 243 - DNA, 39 bp, Artificial Sequence
  synthetic polynucleotide antibody CDR
gcgaaggtacagactacggttattactccttttcaaaac

SEQ ID NO: 244 - DNA, 21 bp, Artificial Sequence
  synthetic polynucleotide antibody CDR
cagaatattggcagcaacttc

SEQ ID NO: 245 - DNA, 9 bp, Artificial Sequence
  synthetic polynucleotide antibody CDR
ggtgcgtcc

SEQ ID NO: 246 - DNA, 27 bp, Artificial Sequence
  synthetic polynucleotide antibody CDR
cagcagtatgataactcaccgtacact

SEQ ID NO: 247 - DNA, 24 bp, Artificial Sequence
  synthetic polynucleotide antibody CDR
ggattcaccttcagtagctatgct

SEQ ID NO: 248 - DNA, 24 bp, Artificial Sequence
  synthetic polynucleotide antibody CDR
atatcatatgatggaagcaaaaaa

SEQ ID NO: 249 - DNA, 36 bp, Artificial Sequence
  synthetic polynucleotide antibody CDR
gcgctcttgtcccgtccacactacggtttggacgtc

SEQ ID NO: 250 - DNA, 24 bp, Artificial Sequence
  synthetic polynucleotide antibody CDR
cgctccaacgttgggggtaatttt

SEQ ID NO: 251 - DNA, 9 bp, Artificial Sequence
  synthetic polynucleotide antibody CDR
gacaattat

SEQ ID NO: 252 - DNA, 33 bp, Artificial Sequence
  synthetic polynucleotide antibody CDR
ggaacatgggatagcagcctgagttcttgggtg

SEQ ID NO: 253 - protein, 121 aa, Artificial Sequence
  synthetic polypeptide antibody heavy chain
EVQLVESGGGLVQPGRSLRLSCAASGFTFSSYAMHWVRQAPGKGLEWVAVISYDGSNKYYADSVKGRFTISRDNSKNTLYLQMNSLRAEDTAVYYCAKDQYSVGATTYDYWGQGTLVTVSS

SEQ ID NO: 254 - protein, 112 aa, Artificial Sequence
  synthetic polypeptide antibody light chain
QPVLTQSSSASASLGASVKLTCSLSSGHSSYAIAWHQQQPEKGPRYLMKLNSDGSHSKGDGIPDRFSGSSSGAERYLTISSLQSEDEADYYCQTWGSGIQVFGGGTKLTVLG

SEQ ID NO: 255 - protein, 118 aa, Artificial Sequence
  synthetic polypeptide antibody heavy chain
EVQLVESGGGLVQPGRSLRLSCAASGFTFSSYAMHWVRQAPGKGLEWVAVISYDGSNKYYADSVKGRFTISRDNSKNTLYLQMNSLRAEDTAVYYCASKRELHSFDYWGQGTLVTVSS

SEQ ID NO: 256 - protein, 113 aa, Artificial Sequence
  synthetic polypeptide antibody light chain
DVVMTQSPLSLPVTPGEPASISCRSSQSLLQSNGHNYLNWYLQKPGQSPQVLIYLASNRASGVPDRFSGSGSGTDFTLKISRVEAEDVGIYYCMQGLQIPLTFGGGTKVEIKR

SEQ ID NO: 257 - protein, 122 aa, Artificial Sequence
  synthetic polypeptide antibody heavy chain
QITLKESGPTLVKPTQTLTLTCTFSGFSLSTSGVGVGWIRQPPGKALEWLALIYWDDDKRYSPSLKSRLTITKDTSKNQVVLTMTNMDPVDTATYYCAHSGSVGYALYFDYWGQGTLVTVSS

SEQ ID NO: 258 - protein, 117 aa, Artificial Sequence
  synthetic polypeptide antibody light chain
QAVLTQPPSSSASPGESARLTCTLPSDINVRYYNIYWYQQKPGSPPRYLLYYQSDSHKGRGSGVPSRFSGSKDTSANTGILLISGLQSEDEADYYCMIWASNGSGVLGGGTQLTVLG

SEQ ID NO: 259 - protein, 126 aa, Artificial Sequence
  synthetic polypeptide antibody heavy chain
QVQLVQSGAEVKKPGTSVKVSCKASGYTFTTYTMHWVRQAPGQRLEWMGWINTGNGNTKYSQKFQDRVTITRDTSASTAYMELSSLKFEDTAVYYCAREGVTIFGDHSYYYGMDVWGQGTTVTVSS

SEQ ID NO: 260 - protein, 112 aa, Artificial Sequence
  synthetic polypeptide antibody light chain
QPVLTQSSSASASLGSSVKLTCTLSSGHSSYIIAWHQQQPGKAPRYLMKLEGSGSYNKGSGLPDRFSGSSSGADRYLTISNLQSEDEADYYCETWDGNTWVFGGGTQLTVLG

SEQ ID NO: 261 - protein, 123 aa, Artificial Sequence
  synthetic polypeptide antibody heavy chain
EVQLVESGGGVVKPGESLRLLCTASGLSFNKYSINWVRQAPGGGLEWVSSIESGSGHIYYADSLEGRFTISRDNAKNSVTLEMNSLRVEDTALYYCVSGPEDKWLLQLYFESWGQGTLVTVSS

SEQ ID NO: 262 - protein, 109 aa, Artificial Sequence
  synthetic polypeptide antibody light chain
EIVLTQSPATLSLSPGERATLSCRTSQSSPSDNLAWYQHKPGQAPRLLIYGGSNRATGIPARFSGSGSGTDFTLTISSLEPEDFAVYYCQQYGSSPLTFGGGTKVDIKR

SEQ ID NO: 263 - protein, 118 aa, Artificial Sequence
  synthetic polypeptide antibody heavy chain
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGWRGGSFMDVWGQGTTVTVSS

SEQ ID NO: 264 - protein, 110 aa, Artificial Sequence
  synthetic polypeptide antibody light chain
QSALTQPRSVSGSPGQSVTISCTGTSSDVGGYNYVSWYQQHPGKAPKLMIYDVIKRPSGVPDRFSGSRSGNTASLTISGLQAEDEADYYCCSYAGTHWVFGGGTKLTVLG

SEQ ID NO: 265 - protein, 121 aa, Artificial Sequence
  synthetic polypeptide antibody heavy chain
EVQLVESGGGLVQPGGSLRLSCAASGFTFSSYAMTWVRQAPGKGLEWVSVISGGGGTTYYADSVKGRFTISRDNSKNTLYLQMSSLRAEDTAVYYCAKGYSSGWPYYFDYWGQGTLVTVSS

SEQ ID NO: 266 - protein, 109 aa, Artificial Sequence
  synthetic polypeptide antibody light chain
EIVLTQSPGTLSLSPGERATLSCRASQSGVTNFLAWYQQKPGQAPRLLIYATSSRATGIPDRFSGSASGTDFTLTITRLEPEDFAVYFCQQYASSPLTFGGGTKVEIKR

SEQ ID NO: 267 - protein, 122 aa, Artificial Sequence
  synthetic polypeptide antibody heavy chain
QVQLVQSGAEVKKPGESLKISCKGSGYDFNNYWIGWVRQTPEKGLEWMGIVYPGDHPGDYHIRYGPSFQGQVTISADRSITTAYLQWRNLKASDTAMYYCARLGSSKDLDYWGQGTLVTVSS

SEQ ID NO: 268 - protein, 108 aa, Artificial Sequence
  synthetic polypeptide antibody light chain
EIVLTQSPATLSLSPGERATLSCRASQSVSGHLDWYQQKPGQSPRLLIYDASNRATGIPARFSGSGSGTDFSLTISSLEPEDFAVYYCQHRSNWPWTFGQGTKVEIKR

SEQ ID NO: 269 - protein, 119 aa, Artificial Sequence
  synthetic polypeptide antibody heavy chain
QVQLQESGPGLMKPSQTLSLTCTVSGDSITRGSSYWSWIRQSDGKGLEWIGHIYSGGDTDYNPALKSRVTISADASRGQFLLRLTSMTAADTAVYYCARDRGAYGMDVWGQGTTVTVSS

SEQ ID NO: 270 - protein, 112 aa, Artificial Sequence
  synthetic polypeptide antibody light chain
QPVLTQSSSASASLGASVKLTCTLSSEHDRYAIAWLQQKPEKGPRYLMKVNSDGSHRKGDGIPDRFSGSSSGAERYLTISRLQSEDEADYYCQTWGIGIRVFGGGTQLTVLG

SEQ ID NO: 271 - protein, 126 aa, Artificial Sequence
  synthetic polypeptide antibody heavy chain
QVQLVESGGGVVQPGRSRRLSCAASGFAFRNYGMHWVRQAPGKGLEWVAFISQNGGKKYYADSVTGRFTVSRDNSKNTLYLQINSLTTDDTAVFYCARSGSGSWGYSDFPGPFDHWGQGSLVTVSS

SEQ ID NO: 272 - protein, 109 aa, Artificial Sequence
  synthetic polypeptide antibody light chain
EIVLTQSPGTLSLSPGERATLSCRASQNIFISFLAWYQQKPGQAPRLLLYGASNRATGIPDRFSGSGSGTDFTLTISRLEPEDFAVYYCQQYGSSPLTFGGGTKVEIKR

SEQ ID NO: 273 - protein, 120 aa, Artificial Sequence
  synthetic polypeptide antibody heavy chain
QVQLVESGGGVVQPGGSLKLSCVASGFTFSAYAMNWVRLVPGKGLEWVSGISGNGYSTFYPDSVQGRFTVSRDNSKNTLFLQIDRLTGGDTAIYYCAKVQTTVITPFQNWGQGTLVTVSS

SEQ ID NO: 274 - protein, 109 aa, Artificial Sequence
  synthetic polypeptide antibody light chain
EIVLTQSPGTLSLSPGERATLSCRASQNIGSNFLAWYQQKPGQAPRLLIYGASTRANGIPDRFSGSKSETDFTLTISRLEPEDFAVYYCQQYDNSPYTFGQGTKLEIKR

SEQ ID NO: 275 - protein, 119 aa, Artificial Sequence
  synthetic polypeptide antibody heavy chain
EVQLVEIGGGLIQPGRSLRLSCAASGFTFSSYAMHWVRQAPGKGLEWVAVISYDGSKKYYADSVKGRFTISRDNSKNTVHLQMNSLRVEDTAVYYCALLSRPHYGLDVWGQGTTVTVSS

SEQ ID NO: 276 - protein, 111 aa, Artificial Sequence
  synthetic polypeptide antibody light chain
QSVLTQPPSVSAAPGQKVTISCSGSRSNVGGNFLSWYQHVPGTPPQLLIYDNYKRPSEIPDRFSGSKSGTSATLDITGLQTGDEADYYCGTWDSSLSSWVFGGGTQLTVLG

SEQ ID NO: 277 - protein, 8 aa, Artificial Sequence
  synthetic polypeptide antibody CDR
GFTFSSYA

SEQ ID NO: 278 - protein, 8 aa, Artificial Sequence
  synthetic polypeptide antibody CDR
ISYDGSNK

SEQ ID NO: 279 - protein, 14 aa, Artificial Sequence
  synthetic polypeptide antibody CDR
AKDQYSVGATTYDY

SEQ ID NO: 280 - protein, 7 aa, Artificial Sequence
  synthetic polypeptide antibody CDR
SGHSSYA

SEQ ID NO: 281 - protein, 7 aa, Artificial Sequence
  synthetic polypeptide antibody CDR
LNSDGSH

SEQ ID NO: 282 - protein, 9 aa, Artificial Sequence
  synthetic polypeptide antibody CDR
QTWGSGIQV

SEQ ID NO: 283 - protein, 8 aa, Artificial Sequence
  synthetic polypeptide antibody CDR
GFTFSSYA

SEQ ID NO: 284 - protein, 8 aa, Artificial Sequence
  synthetic polypeptide antibody CDR
ISYDGSNK

SEQ ID NO: 285 - protein, 11 aa, Artificial Sequence
  synthetic polypeptide antibody CDR
ASKRELHSFDY

SEQ ID NO: 286 - protein, 11 aa, Artificial Sequence
  synthetic polypeptide antibody CDR
QSLLQSNGHNY

SEQ ID NO: 287 - protein, 3 aa, Artificial Sequence
  synthetic polypeptide antibody CDR
LAS

SEQ ID NO: 288 - protein, 9 aa, Artificial Sequence
  synthetic polypeptide antibody CDR
MQGLQIPLT

SEQ ID NO: 289 - protein, 10 aa, Artificial Sequence
  synthetic polypeptide antibody CDR
GFSLSTSGVG

SEQ ID NO: 290 - protein, 7 aa, Artificial Sequence
  synthetic polypeptide antibody CDR
IYWDDDK

SEQ ID NO: 291 - protein, 14 aa, Artificial Sequence
  synthetic polypeptide antibody CDR
AHSGSVGYALYFDY

SEQ ID NO: 292 - protein, 9 aa, Artificial Sequence
  synthetic polypeptide antibody CDR
SDINVRYYN

SEQ ID NO: 293 - protein, 7 aa, Artificial Sequence
  synthetic polypeptide antibody CDR
YQSDSHK

SEQ ID NO: 294 - protein, 10 aa, Artificial Sequence
  synthetic polypeptide antibody CDR
MIWASNGSGV

SEQ ID NO: 295 - protein, 8 aa, Artificial Sequence
  synthetic polypeptide antibody CDR
GYTFTTYT

SEQ ID NO: 296 - protein, 8 aa, Artificial Sequence
  synthetic polypeptide antibody CDR
INTGNGNT

SEQ ID NO: 297 - protein, 19 aa, Artificial Sequence
  synthetic polypeptide antibody CDR
AREGVTIFGDHSYYYGMDV

SEQ ID NO: 298 - protein, 7 aa, Artificial Sequence
  synthetic polypeptide antibody CDR
SGHSSYI

SEQ ID NO: 299 - protein, 7 aa, Artificial Sequence
  synthetic polypeptide antibody CDR
LEGSGSY

SEQ ID NO: 300 - protein, 9 aa, Artificial Sequence
  synthetic polypeptide antibody CDR
ETWDGNTWV

SEQ ID NO: 301 - protein, 8 aa, Artificial Sequence
  synthetic polypeptide antibody CDR
GLSFNKYS

SEQ ID NO: 302 - protein, 8 aa, Artificial Sequence
  synthetic polypeptide antibody CDR
IESGSGHI

SEQ ID NO: 303 - protein, 16 aa, Artificial Sequence
  synthetic polypeptide antibody CDR
VSGPEDKWLLQLYFES

SEQ ID NO: 304 - protein, 7 aa, Artificial Sequence
  synthetic polypeptide antibody CDR
QSSPSDN

SEQ ID NO: 305 - protein, 3 aa, Artificial Sequence
  synthetic polypeptide antibody CDR
GGS

SEQ ID NO: 306 - protein, 9 aa, Artificial Sequence
  synthetic polypeptide antibody CDR
QQYGSSPLT

SEQ ID NO: 307 - protein, 8 aa, Artificial Sequence
  synthetic polypeptide antibody CDR
GGSFSGYY

SEQ ID NO: 308 - protein, 7 aa, Artificial Sequence
  synthetic polypeptide antibody CDR
INHSGST

SEQ ID NO: 309 - protein, 12 aa, Artificial Sequence
  synthetic polypeptide antibody CDR
ARGWRGGSFMDV

SEQ ID NO: 310 - protein, 9 aa, Artificial Sequence
  synthetic polypeptide antibody CDR
SSDVGGYNY

SEQ ID NO: 311 - protein, 3 aa, Artificial Sequence
  synthetic polypeptide antibody CDR
DVI

SEQ ID NO: 312 - protein, 9 aa, Artificial Sequence
  synthetic polypeptide antibody CDR
CSYAGTHWV

SEQ ID NO: 313 - protein, 8 aa, Artificial Sequence
  synthetic polypeptide antibody CDR
GFTFSSYA

SEQ ID NO: 314 - protein, 8 aa, Artificial Sequence
  synthetic polypeptide antibody CDR
ISGGGGTT

SEQ ID NO: 315 - protein, 14 aa, Artificial Sequence
  synthetic polypeptide antibody CDR
AKGYSSGWPYYFDY

SEQ ID NO: 316 - protein, 7 aa, Artificial Sequence
  synthetic polypeptide antibody CDR
QSGVTNF

SEQ ID NO: 317 - protein, 3 aa, Artificial Sequence
  synthetic polypeptide antibody CDR
ATS

SEQ ID NO: 318 - protein, 9 aa, Artificial Sequence
  synthetic polypeptide antibody CDR
QQYASSPLT

SEQ ID NO: 319 - protein, 8 aa, Artificial Sequence
  synthetic polypeptide antibody CDR
GYDFNNYW

SEQ ID NO: 320 - protein, 12 aa, Artificial Sequence
  synthetic polypeptide antibody CDR
VYPGDHPGDYHI

SEQ ID NO: 321 - protein, 11 aa, Artificial Sequence
  synthetic polypeptide antibody CDR
ARLGSSKDLDY

SEQ ID NO: 322 - protein, 6 aa, Artificial Sequence
  synthetic polypeptide antibody CDR
QSVSGH

SEQ ID NO: 323 - protein, 3 aa, Artificial Sequence
  synthetic polypeptide antibody CDR
DAS

SEQ ID NO: 324 - protein, 9 aa, Artificial Sequence
  synthetic polypeptide antibody CDR
QHRSNWPWT

SEQ ID NO: 325 - protein, 10 aa, Artificial Sequence
  synthetic polypeptide antibody CDR
GDSITRGSSY

SEQ ID NO: 326 - protein, 7 aa, Artificial Sequence
  synthetic polypeptide antibody CDR
IYSGGDT

SEQ ID NO: 327 - protein, 11 aa, Artificial Sequence
  synthetic polypeptide antibody CDR
ARDRGAYGMDV

SEQ ID NO: 328 - protein, 7 aa, Artificial Sequence
  synthetic polypeptide antibody CDR
SEHDRYA

SEQ ID NO: 329 - protein, 7 aa, Artificial Sequence
  synthetic polypeptide antibody CDR
VNSDGSH

SEQ ID NO: 330 - protein, 9 aa, Artificial Sequence
  synthetic polypeptide antibody CDR
QTWGIGIRV

SEQ ID NO: 331 - protein, 8 aa, Artificial Sequence
  synthetic polypeptide antibody CDR
GFAFRNYG

SEQ ID NO: 332 - protein, 8 aa, Artificial Sequence
  synthetic polypeptide antibody CDR
ISQNGGKK

SEQ ID NO: 333 - protein, 19 aa, Artificial Sequence
  synthetic polypeptide antibody CDR
ARSGSGSWGYSDFPGPFDH

SEQ ID NO: 334 - protein, 7 aa, Artificial Sequence
  synthetic polypeptide antibody CDR
QNIFISF

SEQ ID NO: 335 - protein, 3 aa, Artificial Sequence
  synthetic polypeptide antibody CDR
GAS

SEQ ID NO: 336 - protein, 9 aa, Artificial Sequence
  synthetic polypeptide antibody CDR
QQYGSSPLT

SEQ ID NO: 337 - protein, 8 aa, Artificial Sequence
  synthetic polypeptide antibody CDR
GFTFSAYA

SEQ ID NO: 338 - protein, 8 aa, Artificial Sequence
  synthetic polypeptide antibody CDR
ISGNGYST

SEQ ID NO: 339 - protein, 13 aa, Artificial Sequence
  synthetic polypeptide antibody CDR
AKVQTTVITPFQN

SEQ ID NO: 340 - protein, 7 aa, Artificial Sequence
  synthetic polypeptide antibody CDR
QNIGSNF

SEQ ID NO: 341 - protein, 3 aa, Artificial Sequence
  synthetic polypeptide antibody CDR
GAS

SEQ ID NO: 342 - protein, 9 aa, Artificial Sequence
  synthetic polypeptide antibody CDR
QQYDNSPYT

SEQ ID NO: 343 - protein, 8 aa, Artificial Sequence
  synthetic polypeptide antibody CDR
GFTFSSYA

SEQ ID NO: 344 - protein, 8 aa, Artificial Sequence
  synthetic polypeptide antibody CDR
ISYDGSKK

SEQ ID NO: 345 - protein, 12 aa, Artificial Sequence
  synthetic polypeptide antibody CDR
ALLSRPHYGLDV

SEQ ID NO: 346 - protein, 8 aa, Artificial Sequence
  synthetic polypeptide antibody CDR
RSNVGGNF

SEQ ID NO: 347 - protein, 3 aa, Artificial Sequence
  synthetic polypeptide antibody CDR
DNY

SEQ ID NO: 348 - protein, 11 aa, Artificial Sequence
  synthetic polypeptide antibody CDR
GTWDSSLSSWV

SEQ ID NO: 349 - protein, 171 aa, Homo Sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLHFVAVILAFLAAVAAVLISLMWVCNKCAYKFQRKRRHKLKGNPLGLVIIHEWF